(19) United States
(12) Patent Application Publication
(10) Pub. No.: US 2026/0159539 A1
(43) Pub. Date: Jun. 11, 2026

(54) MACROCYCLIC RAS INHIBITORS

(71) Applicant: Revolution Medicines, Inc., Redwood City, CA (US)

(72) Inventors: John E. KNOX, Emerald Hills, CA (US); Elena S. Koltun, Foster City, CA (US); Bernadette K. Latimer, San Mateo, CA (US); Nguyen Hong Quang Luu, San Mateo, CA (US); Benjamin D. Madej, San Leandro, CA (US); Dylan E. Parsons, San Carlos, CA (US); Severin Thompson, Belmont, CA (US); Aidan Tomlinson, San Francisco, CA (US); Naing Aay, San Mateo, CA (US); Andreas Buckl, Basel (CH); G. Leslie Burnett, Redwood City, CA (US); James Cregg, Belmont, CA (US); Adrian L. Gill, Atherton, CA (US); Micah Gliedt, Fremont, CA (US)

(21) Appl. No.: 19/350,374

(22) Filed: Oct. 6, 2025

Related U.S. Application Data
(63) Continuation of application No. PCT/US2024/023208, filed on Apr. 5, 2024.
(60) Provisional application No. 63/457,859, filed on Apr. 7, 2023.

(51) Int. Cl. C07F 9/6561; A61K 31/504; A61K 31/5377; A61K 31/5383; A61K 31/675; A61K 31/695; C07B 59/00; C07D 498/22; C07D 513/22; C07D 519/00; C07F 7/08

(52) U.S. Cl. CPC C07F 9/6561; A61K 31/504; A61K 31/5377; A61K 31/5383; A61K 31/675; A61K 31/695; C07B 59/002; C07D 498/22; C07D 513/22; C07D 519/00; C07F 7/0812; C07B 2200/05

(57) ABSTRACT

The disclosure features macrocyclic compounds, and pharmaceutical compositions and protein complexes thereof, capable of inhibiting Ras proteins, and their uses in the treatment of cancers.

### BACKGROUND

The vast majority of small molecule drugs act by binding a functionally important pocket on a target protein, thereby modulating the activity of that protein. For example, cholesterol-lowering drugs known as statins bind the enzyme active site of HMG-CoA reductase, thus preventing the enzyme from engaging with its substrates. The fact that many such drug/target interacting pairs are known may have misled some into believing that a small molecule modulator could be discovered for most, if not all, proteins provided a reasonable amount of time, effort, and resources. This is far from the case. Current estimates are that only about 10% of all human proteins are targetable by small molecules. Bojadzic and Buchwald, Curr Top Med Chem 18: 674-699 (2019). The other 90% are currently considered refractory or intractable toward above-mentioned small molecule drug discovery. Such targets are commonly referred to as “undruggable.” These undruggable targets include a vast and largely untapped reservoir of medically important human proteins. Thus, there exists a great deal of interest in discovering new molecular modalities capable of modulating the function of such undruggable targets.

It has been well established in literature that Ras proteins (K-Ras, H-Ras, and N-Ras) play an essential role in various human cancers and are therefore appropriate targets for anticancer therapy. Indeed, mutations in Ras proteins account for approximately 30% of all human cancers in the United States, many of which are fatal. Dysregulation of Ras proteins by activating mutations, overexpression or upstream activation is common in human tumors, and activating mutations in Ras are frequently found in human cancer. For example, activating mutations at codon 12 in Ras proteins function by inhibiting both GTPase-activating protein (GAP)-dependent and intrinsic hydrolysis rates of GTP, significantly skewing the population of Ras mutant proteins to the “on” (GTP-bound) state (Ras(ON)), leading to oncogenic MAPK signaling. Notably, Ras exhibits a picomolar affinity for GTP, enabling Ras to be activated even in the presence of low concentrations of this nucleotide. Mutations at codons 13 (e.g., G13C) and 61 (e.g., Q61H or Q61K) of Ras are also responsible for oncogenic activity in some cancers.

Despite extensive drug discovery efforts against Ras during the last several decades, only two agents targeting the K-Ras G12C mutant have been approved in the U.S. (sotorasib and adagrasib). Additional efforts are needed to uncover additional medicines for cancers driven by the various Ras mutations.

### SUMMARY

Provided herein are Ras inhibitors. The approach described herein entails formation of a high affinity three-component complex, or conjugate, between a synthetic ligand and two intracellular proteins which do not interact under normal physiological conditions: the target protein of interest (e.g., Ras), and a widely expressed cytosolic chaperone (presenter protein) in the cell (e.g., cyclophilin A). More specifically, in some embodiments, the inhibitors of Ras described herein induce a new binding pocket in Ras by driving formation of a high affinity tri-complex, or conjugate, between the Ras protein and the widely expressed cytosolic chaperone, cyclophilin A (CYPA). Without being bound by theory, the inventors believe that one way the inhibitory effect on Ras is effected by compounds of the invention and the complexes, or conjugates, they form is by steric occlusion of the interaction site between Ras and downstream effector molecules, such as RAF and PI3K, which are required for propagating the oncogenic signal.

As such, in an aspect, the disclosure features a compound having the structure of Formula Ia or Formula Ib:

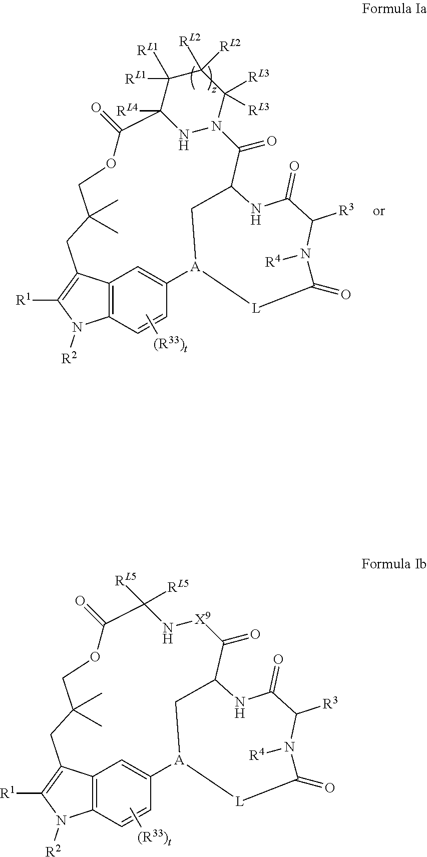

or pharmaceutically acceptable salt, an enantiomer, a stereoisomer, or a tautomer thereof, wherein
A is optionally substituted 3- to 6-membered heterocycloalkylene, optionally substituted 3- to 6-membered cycloalkylene, optionally substituted 6-membered arylene, or optionally substituted 5- to 10-membered heteroarylene;
L is a linker;
R¹ is optionally substituted 5- to 10-membered heteroaryl;
R² is optionally substituted C₁-C₆ alkyl;
R³ is optionally substituted C₁-C₆ alkyl, optionally substituted C₁-C₃ heteroalkyl, or optionally substituted 3- to 6-membered cycloalkyl;
R⁴ is hydrogen or optionally substituted C₁-C₆ alkyl;
each R³³ is, independently, halogen, optionally substituted C₁-C₃ alkyl, optionally substituted C₁-C₃ alkoxy, optionally substituted 3 to 6-membered cycloalkyl, or optionally substituted 3 to 6-membered heterocycloalkyl;
t is 0, 1, 2, or 3;
z is 0, 1, or 2;
X⁹ is —NR^{L6}—, —C(O)—, or —S(O)₂—; and
each of R^{L1}, R^{L2}, R^{L3}, R^{L4}, R^{L4}, R^{L5}, and R^{L6} is, independently, hydrogen, halogen, hydroxyl, optionally substituted C₁-C₆ alkyl, optionally substituted C₂-C₆ alkenyl, optionally substituted C₂-C₆ alkynyl, or optionally substituted C₁-C₆ heteroalkyl; or any two of R^{L1}, R^{L2}, R^{L3}, R^{L4}, R^{L4}, R^{L5} and R^{L6} together with the atoms to which they are attached and any intervening atoms to form an optionally substituted C₃-C₈ cycloalkyl or a 3- to 8-membered heterocyclyl.

In some embodiments, the disclosure features a compound of structural Formula Ia-2:

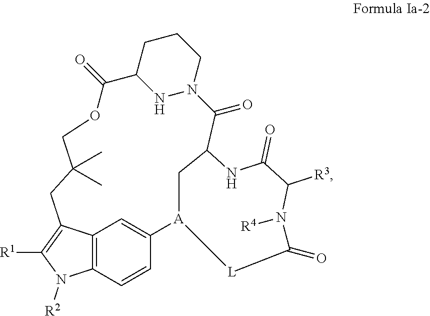

or pharmaceutically acceptable salt, an enantiomer, a stereoisomer, or a tautomer thereof, wherein
A is optionally substituted 3- to 6-membered heterocycloalkylene, optionally substituted 3- to 6-membered cycloalkylene, optionally substituted 6-membered arylene, or optionally substituted 5- to 10-membered heteroarylene;
L is a linker;
R¹ is optionally substituted 5- to 10-membered heteroaryl;
R² is optionally substituted C₁-C₆ alkyl;
R³ is optionally substituted C₁-C₆ alkyl, optionally substituted C₁-C₃ heteroalkyl, or optionally substituted 3- to 6-membered cycloalkyl; and
R⁴ is hydrogen or optionally substituted C₁-C₆ alkyl.

Also provided are pharmaceutical compositions comprising a compound of Formula I, or a pharmaceutically acceptable salt thereof, and a pharmaceutically acceptable excipient. Also provided are pharmaceutical compositions comprising a compound of selected from Tables 1 and 2, or a pharmaceutically acceptable salt thereof, and a pharmaceutically acceptable excipient.

Also provided is a method of treating cancer in a subject in need thereof, the method comprising administering to the subject a therapeutically effective amount of a compound of the present invention, or a pharmaceutically acceptable salt thereof.

In some embodiments, a method is provided of treating a Ras protein-related disorder in a subject in need thereof, the method comprising administering to the subject a therapeutically effective amount of a compound of the present invention, or a pharmaceutically acceptable salt thereof.

Further provided is a method of inhibiting a Ras protein in a cell, the method comprising contacting the cell with an effective amount of a compound of the present invention, or a pharmaceutically acceptable salt thereof.

It is specifically contemplated that any limitation discussed with respect to one embodiment of the invention may apply to any other embodiment of the invention. Furthermore, any compound or composition of the invention may be used in any method of the invention, and any method of the invention may be used to produce or to utilize any compound or composition of the invention.

### Definitions and Chemical Terms

In this application, unless otherwise clear from context, (i) the term “a” means “one or more”; (ii) the term “or” is used to mean “and/or” unless explicitly indicated to refer to alternatives only or the alternative are mutually exclusive, although the disclosure supports a definition that refers to only alternatives and “and/or”; (iii) the terms “comprising” and “including” are understood to encompass itemized components or steps whether presented by themselves or together with one or more additional components or steps; and (iv) where ranges are provided, endpoints are included.

As used herein, the term “about” is used to indicate that a value includes the standard deviation of error for the device or method being employed to determine the value. In certain embodiments, the term “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of a stated value, unless otherwise stated or otherwise evident from the context (e.g., where such number would exceed 100% of a possible value).

As used herein, the term “adjacent” in the context of describing adjacent atoms refers to bivalent atoms that are directly connected by a covalent bond.

A “compound of the present invention” and similar terms as used herein, whether explicitly noted or not, refers to Ras inhibitors described herein, including compounds of Formula I and subformula thereof, for example, a compound of Table 1 or Table 2, as well as salts (e.g., pharmaceutically acceptable salts), solvates, hydrates, stereoisomers (including atropisomers), and tautomers thereof.

The term “wild-type” refers to an entity having a structure or activity as found in nature in a “normal” (as contrasted with mutant, diseased, altered, etc.) state or context. Those of ordinary skill in the art will appreciate that wild-type genes and polypeptides often exist in multiple different forms (e.g., alleles).

Those skilled in the art will appreciate that certain compounds described herein can exist in one or more different isomeric (e.g., stereoisomers, geometric isomers, atropisomers, tautomers) or isotopic (e.g., in which one or more atoms has been substituted with a different isotope of the atom, such as hydrogen substituted for deuterium) forms. Unless otherwise indicated or clear from context, a depicted structure can be understood to represent any such isomeric or isotopic form, individually or in combination.

Compounds described herein can be asymmetric (e.g., having one or more stereocenters). All stereoisomers, such as enantiomers and diastereomers, are intended unless otherwise indicated. Compounds of the present disclosure that contain asymmetrically substituted carbon atoms can be isolated in optically active or racemic forms. Methods on how to prepare optically active forms from optically active starting materials are known in the art, such as by resolution of racemic mixtures or by stereoselective synthesis. Many geometric isomers of olefins, C═N double bonds, and the like can also be present in the compounds described herein, and all such stable isomers are contemplated in the present disclosure. Cis and trans geometric isomers of the compounds of the present disclosure are described and may be isolated as a mixture of isomers or as separated isomeric forms.

In some embodiments, one or more compounds depicted herein may exist in different tautomeric forms. As will be clear from context, unless explicitly excluded, references to such compounds encompass all such tautomeric forms. In some embodiments, tautomeric forms result from the swapping of a single bond with an adjacent double bond and the concomitant migration of a proton. In certain embodiments, a tautomeric form may be a prototropic tautomer, which is an isomeric protonation states having the same empirical formula and total charge as a reference form. Examples of moieties with prototropic tautomeric forms are ketone-enol pairs, amide-imidic acid pairs, lactam-lactim pairs, amide-imidic acid pairs, enamine-imine pairs, and annular forms where a proton can occupy two or more positions of a heterocyclic system, such as, 1H- and 3H-imidazole, 1H-, 2H- and 4H-1,2,4-triazole, 1H- and 2H-isoindole, and 1H- and 2H-pyrazole. In some embodiments, tautomeric forms can be in equilibrium or sterically locked into one form by appropriate substitution. In certain embodiments, tautomeric forms result from acetal interconversion.

Unless otherwise stated, structures depicted herein are also meant to include compounds that differ only in the presence of one or more isotopically enriched atoms. Exemplary isotopes that can be incorporated into compounds of the present invention include isotopes of hydrogen, carbon, nitrogen, oxygen, phosphorus, sulfur, fluorine, chlorine, and iodine, such as ²H, ³H, ¹¹C, ¹³C, ¹⁴C, ¹³N, ¹⁵N, ¹⁵O, ¹⁷O, ¹⁸O, ³²P, ³³P, ³⁵S, ¹⁸F, ³⁶Cl, ¹²³I and ¹²⁵I. Isotopically labeled compounds (e.g., those labeled with ³H and ¹⁴C) can be useful in compound or substrate tissue distribution assays. Tritiated (i.e., ³H) and carbon-14 (i.e., ¹⁴C) isotopes can be useful for their ease of preparation and detectability. Further, substitution with heavier isotopes such as deuterium (i.e., ²H) may afford certain therapeutic advantages resulting from greater metabolic stability (e.g., increased in vivo half-life or reduced dosage requirements). In some embodiments, one or more hydrogen atoms are replaced by ²H or ³H, or one or more carbon atoms are replaced by ¹³C- or ¹⁴C-enriched carbon. Positron emitting isotopes such as 150 ¹³N, ¹¹C, and ¹⁸F are useful for positron emission tomography (PET) studies to examine substrate receptor occupancy. Preparations of isotopically labelled compounds are known to those of skill in the art. For example, isotopically labeled compounds can generally be prepared by following procedures analogous to those disclosed for compounds of the present invention described herein, by substituting an isotopically labeled reagent for a non-isotopically labeled reagent.

Non-limiting examples of moieties that may contain one or more deuterium substitutions in compounds of the present invention, where any position “R” may be deuterium (D), include

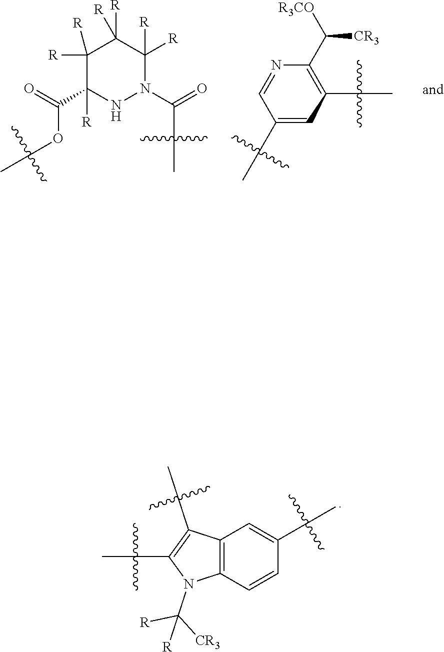

Additional examples include moieties such as

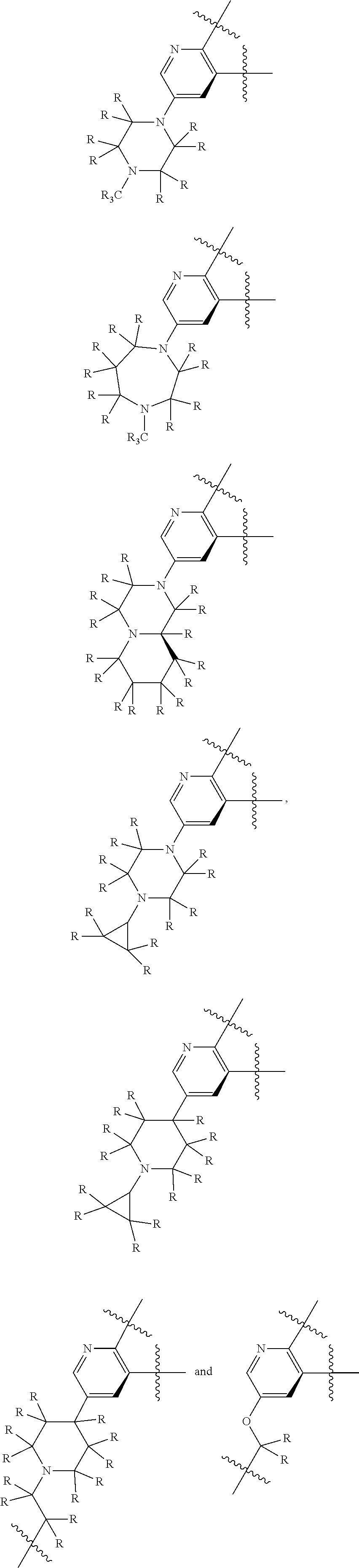

and deuteration of similar R¹-type moieties, wherein the definition of R¹ is found herein (e.g., in compounds of Formula Ia, Ib, Ia-1, Ia-2, IIa, IIb, IIa-1, IIa-2, IIIa, IIIb, IIIa-1, IIIa-2, Va, Vb, Va-1, Va-2, VIIa, VIIb, VIIa-1 or VIIa-2, and subformulae thereof). Moreover, deuteration of available positions in any A moiety of compounds of the Formulas described herein is also contemplated, such as

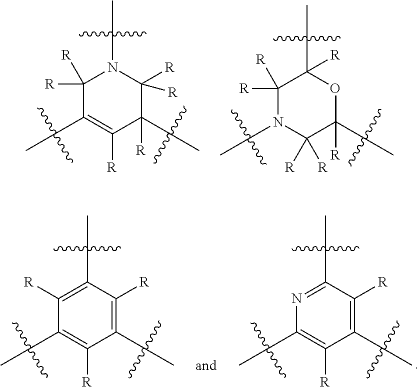

Further, deuterium substitution may also take place in compounds of the present invention at the linker position, such as

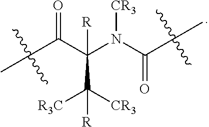

Further, deuterium substitution may also take place in compounds of the present invention at the linker position, such as

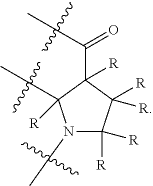

Further, deuterium substitution may also take place in compounds of the present invention at the linker position, such as

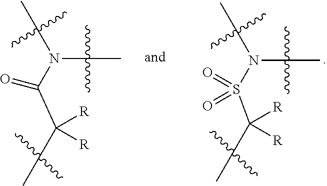

In a further embodiment, silylation substitution is also contemplated, such as in the linker as follows:

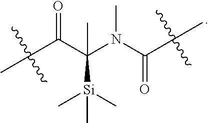 [SMILES: *C(=O)N(C)[C@@](C)(C(*)=O)[Si](C)(C)C]

As is known in the art, many chemical entities can adopt a variety of different solid forms such as, for example, amorphous forms or crystalline forms (e.g., polymorphs, hydrates, solvate). In some embodiments, compounds of the present invention may be utilized in any such form, including in any solid form. In some embodiments, compounds described or depicted herein may be provided or utilized in hydrate or solvate form.

At various places in the present specification, substituents of compounds of the present disclosure are disclosed in groups or in ranges. It is specifically intended that the present disclosure include each and every individual subcombination of the members of such groups and ranges. For example, the term “C₁-C₆ alkyl” is specifically intended to individually disclose methyl, ethyl, C₃ alkyl, C₄ alkyl, C₅ alkyl, and C₆ alkyl. Furthermore, where a compound includes a plurality of positions at which substituents are disclosed in groups or in ranges, unless otherwise indicated, the present disclosure is intended to cover individual compounds and groups of compounds (e.g., genera and subgenera) containing each and every individual subcombination of members at each position.

The term “optionally substituted X” (e.g., “optionally substituted alkyl”) is intended to be equivalent to “X, wherein X is optionally substituted” (e.g., “alkyl, wherein said alkyl is optionally substituted”). It is not intended to mean that the feature “X” (e.g., alkyl) per se is optional. As described herein, certain compounds of interest may contain one or more “optionally substituted” moieties. In general, the term “substituted”, whether preceded by the term “optionally” or not, means that one or more hydrogens of the designated moiety are replaced with a suitable substituent, e.g., any of the substituents or groups described herein. Unless otherwise indicated, an “optionally substituted” group may have a suitable substituent at each substitutable position of the group, and when more than one position in any given structure may be substituted with more than one substituent selected from a specified group, the substituent may be either the same or different at every position. For example, in the term “optionally substituted C₁-C₆ alkyl-C₂-C₈ heteroaryl,” the alkyl portion, the heteroaryl portion, or both, may be optionally substituted. Combinations of substituents envisioned by the present disclosure are preferably those that result in the formation of stable or chemically feasible compounds. The term “stable”, as used herein, refers to compounds that are not substantially altered when subjected to conditions to allow for their production, detection, and, in certain embodiments, their recovery, purification, and use for one or more of the purposes disclosed herein.

Suitable monovalent substituents on a substitutable carbon atom of an “optionally substituted” group may be, independently, deuterium; halogen; —(CH₂)₀₋₄R^{o}; —(CH₂)₀₋₄₀R^{o}; —O(CH₂)₀₋₄R^{o}; —O—(CH₂)₀₋₄C(O)OR^{o}; —(CH₂)₀₋₄CH(OR^{o})₂; —(CH₂)₀₋₄SR^{o}; —(CH₂)₀₋₄Ph, which may be substituted with R^{o}; —(CH₂)₀₋₄₀(CH₂)₀₋₁Ph which may be substituted with R^{o}; —CH═CHPh, which may be substituted with R^{o}; —(CH₂)₀₋₄₀(CH₂)₀₋₁-pyridyl which may be substituted with R^{o}; 4-8 membered saturated or unsaturated heterocycloalkyl (e.g., pyridyl) which may be substituted with R^{o}; 3-8 membered saturated or unsaturated cycloalkyl (e.g., cyclopropyl, cyclobutyl, or cyclopentyl); —NO₂; —CN; —N₃; —(CH₂)₀₋₄N(R^{o})₂; —(CH₂)₀₋₄N(R^{o})C(O)R^{o}; —N(R^{o})C(S)R^{o}; —(CH₂)₀₋₄N(R^{o})C(O)NR^{o}₂; —N(R^{o})C(S)NR^{o}₂; —(CH₂)₀₋₄N(R^{o})C(O)OR^{o}; —N(R^{o})N(R^{o})C(O)R^{o}; —N(R^{o})N(R^{o})C(O)NR^{o}₂; —N(R^{o})N(R^{o})C(O)OR^{o}; —(CH₂)₀₋₄C(O)R^{o}; —C(O)R^{o}, —C(S)R^{o}; —(CH₂)₀₋₄C(O)OR^{o}; —(CH₂)₀₋₄—C(O)—N(R^{o})₂; —(CH₂)₀₋₄—C(O)—N(R^{o})—S(O)₂—R^{o}; —C(NCN)NR^{o}₂; —(CH₂)₀₋₄C(O)SR^{o}; —(CH₂)₀₋₄C(O)OSiR^{o}₃; —(CH₂)₀₋₄OC(O)R^{o}; —OC(O)(CH₂)₀₋₄SR^{o}; —SC(S)SR^{o}; —(CH₂)₀₋₄SC(O)R^{o}; —(CH₂)₀₋₄C(O)NR^{o}₂; —C(S)NR^{o}₂; —C(S)SR^{o}; —(CH₂)₀₋₄OC(O)NR^{o}₂; —C(O)N(OR^{o})R^{o}; —C(O)C(O)R^{o}; —C(O)CH₂C(O)R^{o}; —C(NOR^{o})R^{o}; —(CH₂)₀₋₄SSR^{o}; —(CH₂)₀₋₄S(O)₂R^{o}; —(CH₂)₀₋₄S(O)₂OR^{o}; —(CH₂)₀₋₄OS(O)₂R^{o}; —S(O)₂NR^{o}₂; —(CH₂)₀₋₄S(O)R^{o}; —N(R^{o})S(O)₂NR^{o}₂; —N(R^{o})S(O)₂R^{o}; —N(OR^{o})R^{o}; —C(NOR^{o})NR^{o}₂; —C(NH)NR^{o}₂; —P(O)₂R^{o}; —P(O)R^{o}₂; —P(O)(OR^{o})₂; —OP(O)R^{o}₂; —OP(O)(OR^{o})₂; —OP(O)(OR^{o})R^{o}, —SiR^{o}₃; —(C₁₋₄ straight or branched alkylene)O—N(R^{o})₂; or —(C₁₋₄ straight or branched alkylene)C(O)O—N(R^{o})₂, wherein each R^{o} may be substituted as defined below and is independently hydrogen, —C₁₋₆ aliphatic, —CH₂Ph, —O(CH₂)₀₋₁Ph, —CH₂-(5-6 membered heteroaryl ring), 3-8 membered saturated or unsaturated cycloalkyl (e.g., cyclopropyl, cyclobutyl, or cyclopentyl), or a 3-6-membered saturated, partially unsaturated, or aryl ring having 0-4 heteroatoms independently selected from nitrogen, oxygen, or sulfur, or, notwithstanding the definition above, two independent occurrences of R^{o}, taken together with their intervening atom(s), form a 3-12-membered saturated, partially unsaturated, or aryl mono- or bicyclic ring having 0-4 heteroatoms independently selected from nitrogen, oxygen, or sulfur, which may be substituted as defined below.

Suitable monovalent substituents on R^{o} (or the ring formed by taking two independent occurrences of R^{o} together with their intervening atoms), may be, independently, halogen, —(CH₂)₀₋₂R^{●}, -(haloR^{●}), —(CH₂)₀₋₂OH, —(CH₂)₀₋₂OR^{●}, —(CH₂)₀₋₂CH(OR^{●})₂; —O(haloR^{●}), —CN, —N₃, —(CH₂)₀₋₂C(O)R^{●}, —(CH₂)₀₋₂C(O)OH, —(CH₂)₀₋₂C(O)OR^{●}, —(CH₂)₀₋₂SR^{●}, —(CH₂)₀₋₂SH, —(CH₂)₀₋₂NH₂, —(CH₂)₀₋₂NH R^{●}, —(CH₂)₀₋₂NR^{●}₂, —NO₂, —SiR^{●}₃, —OSiR^{●}₃, —C(O)SR^{●}, —(C₁₋₄ straight or branched alkylene)C(O)OR*, or —SSR^{●} wherein each R^{●} is unsubstituted or where preceded by “halo” is substituted only with one or more halogens, and is independently selected from C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur. Suitable divalent substituents on a saturated carbon atom of R^{o} include ═O and =S.

Suitable divalent substituents on a saturated carbon atom of an “optionally substituted” group include the following: ═O, ═S, ═NNR*₂, =NNHC(O)R*, =NNHC(O)OR*, =NNHS(O)₂R*, =NR*, =NOR*, —O(C(R*₂))₂₋₃O—, or —S(C(R*₂))₂₋₃S—, wherein each independent occurrence of R* is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur. Suitable divalent substituents that are bound to vicinal substitutable carbons of an “optionally substituted” group include: —O(CR*₂)₂₋₃O—, wherein each independent occurrence of R* is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur.

Suitable substituents on the aliphatic group of R* include halogen, —R^{●}, -(haloR^{●}), —OH, —OR^{●}, —O(haloR^{●}), —CN, —C(O)OH, —C(O)OR^{●}, —NH₂, —NHR^{●}, —NR^{●}₂, or —NO₂, wherein each R^{●} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur.

Suitable substituents on a substitutable nitrogen of an “optionally substituted” group include —R^{†}, —NR^{†}₂, —C(O)R^{†}, —C(O)OR^{†}, —C(O)C(O)R^{†}, —C(O)CH₂C(O)R^{†}, —S(O)₂R^{†}, —S(O)₂NR^{†}₂, —C(S)NR^{†}₂, —C(NH)NR^{†}₂, or —N(R^{†})S(O)₂R^{†}; wherein each Rt is independently hydrogen, C₁₋₆ aliphatic which may be substituted as defined below, unsubstituted —OPh, or an unsubstituted 3-6-membered saturated, partially unsaturated, or aryl ring having 0-4 heteroatoms independently selected from nitrogen, oxygen, or sulfur, or, notwithstanding the definition above, two independent occurrences of R^{†}, taken together with their intervening atom(s) form an unsubstituted 3-12-membered saturated, partially unsaturated, or aryl mono- or bicyclic ring having 0-4 heteroatoms independently selected from nitrogen, oxygen, or sulfur.

Suitable substituents on an aliphatic group of Rt are independently halogen, —R^{●}, -(haloR^{●}), —OH, —OR^{●}, —O(haloR^{●}), —CN, —C(O)OH, —C(O)OR^{●}, —NH₂, —NHR^{●}, —NR*₂, or —NO₂, wherein each R^{●} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur. Suitable divalent substituents on a saturated carbon atom of Rt include ═O and =S.

The term “acetyl,” as used herein, refers to the group —C(O)CH₃.

The term “alkoxy,” as used herein, refers to a —O—C₁-C₂₀ alkyl group, wherein the alkoxy group is attached to the remainder of the compound through an oxygen atom.

The term “alkyl,” as used herein, refers to a saturated, straight or branched monovalent hydrocarbon group containing from 1 to 20 (e.g., from 1 to 10 or from 1 to 6) carbons. In some embodiments, an alkyl group is unbranched (i.e., is linear); in some embodiments, an alkyl group is branched. Alkyl groups are exemplified by, but not limited to, methyl, ethyl, n- and iso-propyl, n-, sec-, iso- and tert-butyl, and neopentyl.

The term “alkylene,” as used herein, represents a saturated divalent hydrocarbon group derived from a straight or branched chain saturated hydrocarbon by the removal of two hydrogen atoms, and is exemplified by methylene, ethylene, isopropylene, and the like. The term “Cr-Cy alkylene” represents alkylene groups having between x and y carbons. Exemplary values for x are 1, 2, 3, 4, 5, and 6, and exemplary values for y are 2, 3, 4, 5, 6, 7, 8, 9, 10, 12, 14, 16, 18, or 20 (e.g., C₁-C₆, C₁-C₁₀, C₂-C₂₀, C₂-C₆, C₂-C₁₀, or C₂-C₂₀ alkylene). In some embodiments, the alkylene can be further substituted with 1, 2, 3, or 4 substituent groups as defined herein.

The term “alkenyl,” as used herein, represents monovalent straight or branched chain groups of, unless otherwise specified, from 2 to 20 carbons (e.g., from 2 to 6 or from 2 to 10 carbons) containing one or more carbon-carbon double bonds and is exemplified by ethenyl, 1-propenyl, 2-propenyl, 2-methyl-1-propenyl, 1-butenyl, and 2-butenyl. Alkenyls include both cis and trans isomers. The term “alkenylene,” as used herein, represents a divalent straight or branched chain groups of, unless otherwise specified, from 2 to 20 carbons (e.g., from 2 to 6 or from 2 to 10 carbons) containing one or more carbon-carbon double bonds.

The term “alkynyl,” as used herein, represents monovalent straight or branched chain groups from 2 to 20 carbon atoms (e.g., from 2 to 4, from 2 to 6, or from 2 to 10 carbons) containing a carbon-carbon triple bond and is exemplified by ethynyl, and 1-propynyl.

The term “alkynyl sulfone,” as used herein, represents a group comprising the structure

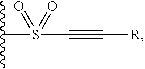

wherein R is any chemically feasible substituent described herein.

The term “amino,” as used herein, represents —N(R^{†})₂, e.g., —NH₂ and —N(CH₃)₂.

The term “aminoalkyl,” as used herein, represents an alkyl moiety substituted on one or more carbon atoms with one or more amino moieties.

The term “amino acid,” as described herein, refers to a molecule having a side chain, an amino group, and an acid group (e.g., —CO₂H or —SO₃H), wherein the amino acid is attached to the parent molecular group by the side chain, amino group, or acid group (e.g., the side chain). As used herein, the term “amino acid” in its broadest sense, refers to any compound or substance that can be incorporated into a polypeptide chain, e.g., through formation of one or more peptide bonds. In some embodiments, an amino acid has the general structure H₂N—C(H)(R)—COOH. In some embodiments, an amino acid is a naturally-occurring amino acid. In some embodiments, an amino acid is a synthetic amino acid; in some embodiments, an amino acid is a D-amino acid; in some embodiments, an amino acid is an L-amino acid. “Standard amino acid” refers to any of the twenty standard L-amino acids commonly found in naturally occurring peptides. Exemplary amino acids include alanine, arginine, asparagine, aspartic acid, cysteine, glutamic acid, glutamine, glycine, histidine, optionally substituted hydroxylnorvaline, isoleucine, leucine, lysine, methionine, norvaline, ornithine, phenylalanine, proline, pyrrolysine, selenocysteine, serine, taurine, threonine, tryptophan, tyrosine, and valine.

The term “aryl,” as used herein, represents a monovalent monocyclic, bicyclic, or multicyclic ring system formed by carbon atoms, wherein the ring attached to the pendant group is aromatic. Examples of aryl groups are phenyl, naphthyl, phenanthrenyl, and anthracenyl. An aryl ring can be attached to its pendant group at any heteroatom or carbon ring atom that results in a stable structure and any of the ring atoms can be optionally substituted unless otherwise specified.

The term “C₀,” as used herein, represents a bond. For example, part of the term —N(C(O)—(C₀-C₅ alkylene-H)- includes —N(C(O)—(C₀ alkylene-H)—, which is also represented by —N(C(O)—H)—.

The terms “carbocyclic” and “carbocyclyl,” as used herein, refer to a monovalent, optionally substituted C₃-C₁₂ monocyclic, bicyclic, or tricyclic ring structure, which may be bridged, fused or spirocyclic, in which all the rings are formed by carbon atoms and at least one ring is non-aromatic. Carbocyclic structures include cycloalkyl, cycloalkenyl, and cycloalkynyl groups. Examples of carbocyclyl groups are cyclohexyl, cyclohexenyl, cyclooctynyl, 1,2-dihydronaphthyl, 1,2,3,4-tetrahydronaphthyl, fluorenyl, indenyl, indanyl, decalinyl, and the like. A carbocyclic ring can be attached to its pendant group at any ring atom that results in a stable structure and any of the ring atoms can be optionally substituted unless otherwise specified.

The term “carbonyl,” as used herein, represents a C(O) group, which can also be represented as C═O.

The term “carboxyl,” as used herein, means —CO₂H, (C═O)(OH), COOH, or C(O)OH or the unprotonated counterparts.

The term “cyano,” as used herein, represents a —CN group.

The term “cycloalkyl,” as used herein, represents a monovalent saturated cyclic hydrocarbon group, which may be bridged, fused or spirocyclic having from three to eight ring carbons, unless otherwise specified, and is exemplified by cyclopropyl, cyclobutyl, cyclopentyl, cyclohexyl, cycloheptyl, and cycloheptyl.

The term “cycloalkenyl,” as used herein, represents a monovalent, non-aromatic, saturated cyclic hydrocarbon group, which may be bridged, fused or spirocyclic having from three to eight ring carbons, unless otherwise specified, and containing one or more carbon-carbon double bonds.

The term “diastereomer,” as used herein, means stereoisomers that are not mirror images of one another and are non-superimposable on one another.

The term “enantiomer,” as used herein, means each individual optically active form of a compound of the invention, having an optical purity or enantiomeric excess (as determined by methods standard in the art) of at least 80% (i.e., at least 90% of one enantiomer and at most 10% of the other enantiomer), preferably at least 90% and more preferably at least 98%.

The term “guanidinyl,” refers to a group having the structure:

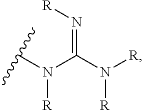

wherein each R is, independently, any chemically feasible substituent described herein.

The term “guanidinoalkyl alkyl,” as used herein, represents an alkyl moiety substituted on one or more carbon atoms with one or more guanidinyl moieties.

The term “haloacetyl,” as used herein, refers to an acetyl group wherein at least one of the hydrogens has been replaced by a halogen.

The term “haloalkyl,” as used herein, represents an alkyl moiety substituted on one or more carbon atoms with one or more of the same of different halogen moieties.

The term “halogen,” as used herein, represents a halogen selected from bromine, chlorine, iodine, or fluorine.

The term “heteroalkyl,” as used herein, refers to an “alkyl” group, as defined herein, in which at least one carbon atom has been replaced with a heteroatom (e.g, an O, N, or S atom). The heteroatom may appear in the middle or at the end of the radical.

The term “heteroaryl,” as used herein, represents a monovalent, monocyclic, or polycyclic ring structure that contains at least one fully aromatic ring: i.e., they contain 4n+2 pi electrons within the monocyclic or polycyclic ring system and contains at least one ring heteroatom selected from N, O, or S in that aromatic ring. Exemplary unsubstituted heteroaryl groups are of 1 to 12 (e.g., 1 to 11, 1 to 10, 1 to 9, 2 to 12, 2 to 11, 2 to 10, or 2 to 9) carbons. The term “heteroaryl” includes bicyclic, tricyclic, and tetracyclic groups in which any of the above heteroaromatic rings is fused to one or more, aryl or carbocyclic rings, e.g., a phenyl ring, or a cyclohexane ring. Examples of heteroaryl groups include, but are not limited to, pyridyl, pyrazolyl, benzooxazolyl, benzoimidazolyl, benzothiazolyl, imidazolyl, thiazolyl, quinolinyl, tetrahydroquinolinyl, and 4-azaindolyl. A heteroaryl ring can be attached to its pendant group at any ring atom that results in a stable structure and any of the ring atoms can be optionally substituted unless otherwise specified. In some embodiments, the heteroaryl is substituted with 1, 2, 3, or 4 substituents groups.

The term “heterocycloalkyl,” as used herein, represents a monovalent monocyclic, bicyclic, or polycyclic ring system, which may be bridged, fused or spirocyclic, wherein at least one ring is non-aromatic and wherein the non-aromatic ring contains one, two, three, or four heteroatoms independently selected from the group consisting of nitrogen, oxygen, and sulfur. The 5-membered ring has zero to two double bonds, and the 6- and 7-membered rings have zero to three double bonds. Exemplary unsubstituted heterocycloalkyl groups are of 1 to 12 (e.g., 1 to 11, 1 to 10, 1 to 9, 2 to 12, 2 to 11, 2 to 10, or 2 to 9) carbons. The term “heterocycloalkyl” also represents a heterocyclic compound having a bridged multicyclic structure in which one or more carbons or heteroatoms bridges two non-adjacent members of a monocyclic ring, e.g., a quinuclidinyl group. The term “heterocycloalkyl” includes bicyclic, tricyclic, and tetracyclic groups in which any of the above heterocyclic rings is fused to one or more aromatic, carbocyclic, heteroaromatic, or heterocyclic rings, e.g., an aryl ring, a cyclohexane ring, a cyclohexene ring, a cyclopentane ring, a cyclopentene ring, a pyridine ring, or a pyrrolidine ring. Examples of heterocycloalkyl groups are pyrrolidinyl, piperidinyl, 1,2,3,4-tetrahydroquinolinyl, decahydroquinolinyl, dihydropyrrolopyridine, and decahydronapthyridinyl. A heterocycloalkyl ring can be attached to its pendant group at any ring atom that results in a stable structure and any of the ring atoms can be optionally substituted unless otherwise specified.

The term “hydroxy,” as used herein, represents a —OH group.

The term “hydroxyalkyl,” as used herein, represents an alkyl moiety substituted on one or more carbon atoms with one or more —OH moieties.

The term “isomer,” as used herein, means any tautomer, stereoisomer, atropisomer, enantiomer, or diastereomer of any compound of the invention. It is recognized that the compounds of the invention can have one or more chiral centers or double bonds and, therefore, exist as stereoisomers, such as double-bond isomers (i.e., geometric E/Z isomers) or diastereomers (e.g., enantiomers (i.e., (+) or (−)) or cis/trans isomers). According to the invention, the chemical structures depicted herein, and therefore the compounds of the invention, encompass all the corresponding stereoisomers, that is, both the stereomerically pure form (e.g., geometrically pure, enantiomerically pure, or diastereomerically pure) and enantiomeric and stereoisomeric mixtures, e.g., racemates. Enantiomeric and stereoisomeric mixtures of compounds of the invention can typically be resolved into their component enantiomers or stereoisomers by well-known methods, such as chiral-phase gas chromatography, chiral-phase high performance liquid chromatography, crystallizing the compound as a chiral salt complex, or crystallizing the compound in a chiral solvent. Enantiomers and stereoisomers can also be obtained from stereomerically or enantiomerically pure intermediates, reagents, and catalysts by well-known asymmetric synthetic methods.

As used herein, the term “linker” refers to a divalent organic moiety connecting a first moiety (e.g., one portion of a macrocycle) to a second moiety (e.g., a second portion of the same macrocycle). In some embodiments, the linker results in a compound capable of achieving an IC50 of 2 μM or less in the Ras-RAF disruption assay protocol provided in the Examples below, and provided here:
The purpose of this biochemical assay is to measure the ability of test compounds to facilitate ternary complex formation between a nucleotide-loaded Ras isoform and cyclophilin A; the resulting ternary complex disrupts binding to a BRAF^{RBD} construct, inhibiting Ras signaling through a RAF effector.
In assay buffer containing 25 mM HEPES pH 7.3, 0.002% Tween20, 0.1% BSA, 100 mM NaCl and 5 mM MgCl₂, tagless cyclophilin A, His6-K-Ras-GMPPNP (or other Ras variant), and GST-BRAF^{RBD} are combined in a 384-well assay plate at final concentrations of 25 μM, 12.5 nM and 50 nM, respectively. Compound is present in plate wells as a 10-point 3-fold dilution series starting at a final concentration of 30 μM. After incubation at 25° C. for 3 hours, a mixture of Anti-His Eu—W1024 and anti-GST allophycocyanin is then added to assay sample wells at final concentrations of 10 nM and 50 nM, respectively, and the reaction incubated for an additional 1.5 hours. TR-FRET signal is read on a microplate reader (Ex 320 nm, Em 665/615 nm). Compounds that facilitate disruption of a Ras:RAF complex are identified as those eliciting a decrease in the TR-FRET ratio relative to DMSO control wells.

This assay may be used to assess selectivity as well. In some embodiments, a compound of the present invention is selective for one or more particular Ras mutants (e.g., K-Ras Q61H) over other Ras mutants or wild-type compared to what is known in the art.

In some embodiments, the linker comprises 20 or fewer linear atoms. In some embodiments, the linker comprises 15 or fewer linear atoms. In some embodiments, the linker comprises 10 or fewer linear atoms. In some embodiments, the linker has a molecular weight of under 500 g/mol. In some embodiments, the linker has a molecular weight of under 400 g/mol. In some embodiments, the linker has a molecular weight of under 300 g/mol. In some embodiments, the linker has a molecular weight of under 200 g/mol. In some embodiments, the linker has a molecular weight of under 100 g/mol. In some embodiments, the linker has a molecular weight of under 50 g/mol.

As used herein, a “monovalent organic moiety” is less than 500 kDa. In some embodiments, a “monovalent organic moiety” is less than 400 kDa. In some embodiments, a “monovalent organic moiety” is less than 300 kDa. In some embodiments, a “monovalent organic moiety” is less than 200 kDa. In some embodiments, a “monovalent organic moiety” is less than 100 kDa. In some embodiments, a “monovalent organic moiety” is less than 50 kDa. In some embodiments, a “monovalent organic moiety” is less than 25 kDa. In some embodiments, a “monovalent organic moiety” is less than 20 kDa. In some embodiments, a “monovalent organic moiety” is less than 15 kDa. In some embodiments, a “monovalent organic moiety” is less than 10 kDa. In some embodiments, a “monovalent organic moiety” is less than 1 kDa. In some embodiments, a “monovalent organic moiety” is less than 500 g/mol. In some embodiments, a “monovalent organic moiety” ranges between 500 g/mol and 500 kDa.

The term “stereoisomer,” as used herein, refers to all possible different isomeric as well as conformational forms which a compound may possess (e.g., a compound of any formula described herein), in particular all possible stereochemically and conformationally isomeric forms, all diastereomers, enantiomers or conformers of the basic molecular structure, including atropisomers. Some compounds of the present invention may exist in different tautomeric forms, all of the latter being included within the scope of the present invention.

The term “sulfonyl,” as used herein, represents an —S(O)₂— group.

The term “thiocarbonyl,” as used herein, refers to a —C(S)— group.

The term “vinyl ketone,” as used herein, refers to a group comprising a carbonyl group directly connected to a carbon-carbon double bond.

The term “vinyl sulfone,” as used herein, refers to a group comprising a sulfonyl group directed connected to a carbon-carbon double bond.

The term “ynone,” as used herein, refers to a group comprising the structure

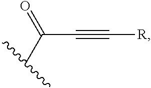

wherein R is any chemically feasible substituent described herein.

Those of ordinary skill in the art, reading the present disclosure, will appreciate that certain compounds described herein may be provided or utilized in any of a variety of forms such as, for example, salt forms, protected forms, pro-drug forms, ester forms, isomeric forms (e.g., optical or structural isomers), isotopic forms, etc. In some embodiments, reference to a particular compound may relate to a specific form of that compound. In some embodiments, reference to a particular compound may relate to that compound in any form. In some embodiments, for example, a preparation of a single stereoisomer of a compound may be considered to be a different form of the compound than a racemic mixture of the compound; a particular salt of a compound may be considered to be a different form from another salt form of the compound; a preparation containing one conformational isomer ((Z) or (E)) of a double bond may be considered to be a different form from one containing the other conformational isomer ((E) or (Z)) of the double bond; a preparation in which one or more atoms is a different isotope than is present in a reference preparation may be considered to be a different form.

### DETAILED DESCRIPTION

### Compounds

Provided herein are Ras inhibitors. The approach described herein entails formation of a high affinity three-component complex, or conjugate, between a synthetic ligand and two intracellular proteins which do not interact under normal physiological conditions: the target protein of interest (e.g., Ras), and a widely expressed cytosolic chaperone (presenter protein) in the cell (e.g., cyclophilin A). More specifically, in some embodiments, the inhibitors of Ras described herein induce a new binding pocket in Ras by driving formation of a high affinity tri-complex, or conjugate, between the Ras protein and the widely expressed cytosolic chaperone, cyclophilin A (CYPA). Without being bound by theory, the inventors believe that one way the inhibitory effect on Ras is effected by compounds of the invention and the complexes, or conjugates, they form is by steric occlusion of the interaction site between Ras and downstream effector molecules, such as RAF, which are required for propagating the oncogenic signal.

Without being bound by theory, the inventors postulate that non-covalent interactions of a compound of the present invention with Ras and the chaperone protein (e.g., cyclophilin A) may contribute to the inhibition of Ras activity. For example, van der Waals, hydrophobic, hydrophilic and hydrogen bond interactions, and combinations thereof, may contribute to the ability of the compounds of the present invention to form complexes and act as Ras inhibitors.

Accordingly, a variety of Ras proteins may be inhibited by a compound of the present invention (e.g., K-Ras, N-Ras, H-Ras, and mutants thereof at positions 12, 13 and 61, such as G12C, G12D, G12V, G12S, G13C, G13D, Q61H, Q61K, Q61R and Q61L, and others described herein, or a combination thereof). In some embodiments, a compound of the present invention inhibits at least a K-Ras Q61H mutant. In some embodiments, a compound of the present invention selectively inhibits at least a K-Ras Q61H mutant compared to wild-type K-Ras. In some embodiments, a compound of the present invention selectively inhibits a K-Ras Q61H mutant compared to wild-type K-Ras and one or more other K-Ras mutants.

In some embodiments, the disclosure features a compound having the structure of Formula Ia or Formula Ib:

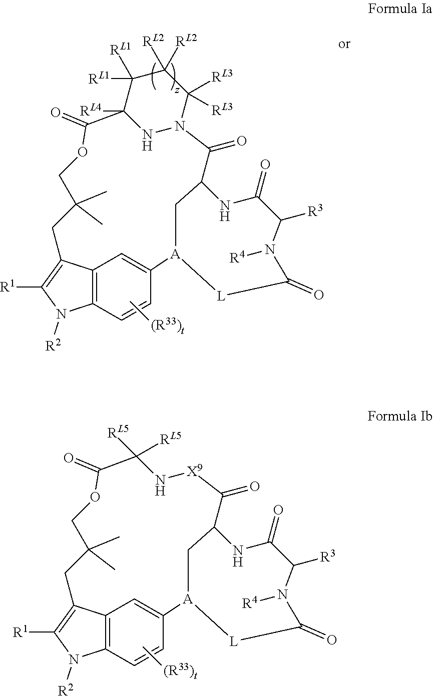

or pharmaceutically acceptable salt, an enantiomer, a stereoisomer, or a tautomer thereof, wherein
A is optionally substituted 3- to 6-membered heterocycloalkylene, optionally substituted 3- to 6-membered cycloalkylene, optionally substituted 6-membered arylene, or optionally substituted 5- to 10-membered heteroarylene;
L is a linker;
R¹ is optionally substituted 5- to 10-membered heteroaryl;
R² is optionally substituted C₁-C₆ alkyl;
R³ is optionally substituted C₁-C₆ alkyl, optionally substituted C₁-C₃ heteroalkyl, or optionally substituted 3- to 6-membered cycloalkyl;
R⁴ is hydrogen or optionally substituted C₁-C₆ alkyl;
each R³³ is, independently, halogen, optionally substituted C₁-C₃ alkyl, optionally substituted C₁-C₃ alkoxy, optionally substituted 3 to 6-membered cycloalkyl, or optionally substituted 3 to 6-membered heterocycloalkyl;
t is 0, 1, 2, or 3;
z is 0, 1, or 2;
X⁹ is —NR^{L6}—, —C(O)—, or —S(O)₂—; and
each of R^{L1}, R^{L2}, R^{L3}, R^{L4}, R^{L4}, R^{L5}, and R^{L6} is, independently, hydrogen, halogen, hydroxyl, optionally substituted C₁-C₆ alkyl, optionally substituted C₂-C₆ alkenyl, optionally substituted C₂-C₆ alkynyl, or optionally substituted C₁-C₆ heteroalkyl; or any two of R^{L1}, R^{L2}, R^{L3}, R^{L4}, R^{L4}, R^{L5}, and R^{L6} together with the atoms to which they are attached and any intervening atoms to form an optionally substituted C₃-C₈ cycloalkyl or a 3- to 8-membered heterocyclyl.

In some embodiments, the disclosure features a compound of structural Formula Ia-1:

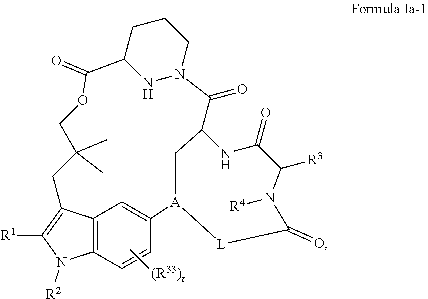

or pharmaceutically acceptable salt, an enantiomer, a stereoisomer, or a tautomer thereof, wherein
A is optionally substituted 3- to 6-membered heterocycloalkylene, optionally substituted 3- to 6-membered cycloalkylene, optionally substituted 6-membered arylene, or optionally substituted 5- to 10-membered heteroarylene;
L is a linker;
R¹ is optionally substituted 5- to 10-membered heteroaryl;
R² is optionally substituted C₁-C₆ alkyl;
R³ is optionally substituted C₁-C₆ alkyl, optionally substituted C₁-C₃ heteroalkyl, or optionally substituted 3- to 6-membered cycloalkyl;
R⁴ is hydrogen or optionally substituted C₁-C₆ alkyl;
each R³³ is, independently, halogen, optionally substituted C₁-C₃ alkyl, optionally substituted C₁-C₃ alkoxy, optionally substituted 3 to 6-membered cycloalkyl, or optionally substituted 3 to 6-membered heterocycloalkyl; and
t is 0, 1, 2, or 3.

In an aspect, the invention features a compound having the structure of Formula Ia-2:

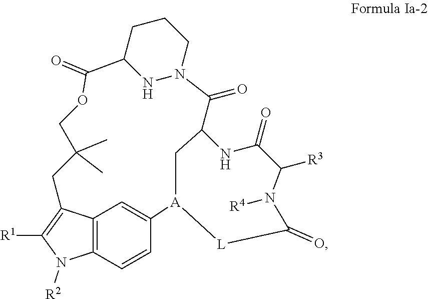

or pharmaceutically acceptable salt, an enantiomer, a stereoisomer, or a tautomer thereof, wherein
A is optionally substituted 3- to 6-membered heterocycloalkylene, optionally substituted 3- to 6-membered cycloalkylene, optionally substituted 6-membered arylene, or optionally substituted 5- to 10-membered heteroarylene;
L is a linker;
R¹ is optionally substituted 5- to 10-membered heteroaryl;
R² is optionally substituted C₁-C₆ alkyl;
R³ is optionally substituted C₁-C₆ alkyl, optionally substituted C₁-C₃ heteroalkyl, or optionally substituted 3- to 6-membered cycloalkyl; and
R⁴ is hydrogen or optionally substituted C₁-C₆ alkyl.

In some embodiments, the compound has the structure of Formula IIa-2:

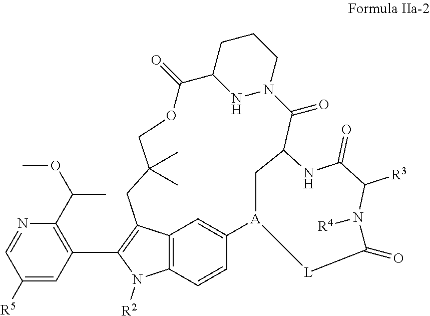

or pharmaceutically acceptable salt, an enantiomer, a stereoisomer, or a tautomer thereof, wherein
R⁵ is hydrogen, optionally substituted C₁-C₆ alkyl, optionally substituted C₁-C₆ alkenyl, optionally substituted C₁-C₆ alkynyl, optionally substituted 3- to 10-membered heterocycloalkyl, —OR^{5a}, or optionally substituted C₁-C₆ heteroalkyl; and
R^{5a} is optionally substituted C₁-C₆ alkyl or optionally substituted 5- to 10-membered heteroaryl.

In some embodiments, R⁵ is hydrogen, optionally substituted 3- to 10-membered heterocycloalkyl, —OR^{5a}, or optionally substituted C₁-C₆ heteroalkyl.

In some embodiments, the disclosure features a compound of structural Formula IIa or Formula IIb:

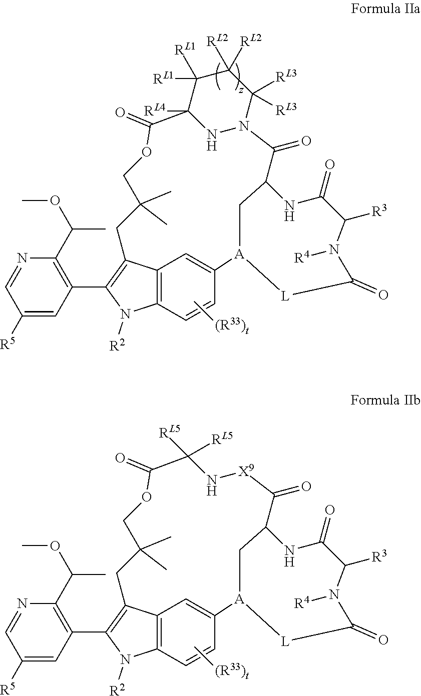

or pharmaceutically acceptable salt, an enantiomer, a stereoisomer, or a tautomer thereof, wherein
R⁵ is hydrogen, optionally substituted 3- to 10-membered heterocycloalkyl, —OR^{5a}, or optionally substituted C₁-C₆ heteroalkyl; and
R^{5a} is optionally substituted C₁-C₆ alkyl or optionally substituted 5- to 10-membered heteroaryl.

In some embodiments, the disclosure features a compound of structural Formula IIa-1:

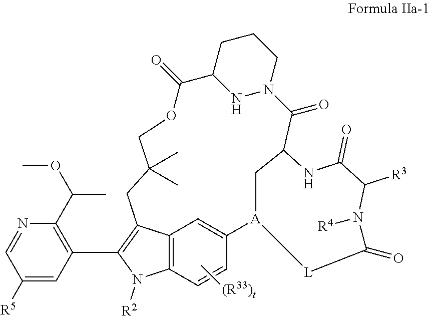

or pharmaceutically acceptable salt, an enantiomer, a stereoisomer, or a tautomer thereof, wherein
R⁵ is hydrogen, optionally substituted 3- to 10-membered heterocycloalkyl, —OR^{5a}, or optionally substituted C₁-C₆ heteroalkyl; and
R^{5a} is optionally substituted C₁-C₆ alkyl or optionally substituted 5- to 10-membered heteroaryl;
each R³³ is, independently, halogen, optionally substituted C₁-C₃ alkyl, optionally substituted C₁-C₃ alkoxy, optionally substituted 3 to 6-membered cycloalkyl, or optionally substituted 3 to 6-membered heterocycloalkyl; and
t is 0, 1, 2, or 3.

In some embodiments, the disclosure features a compound of structural Formula IIa-2:

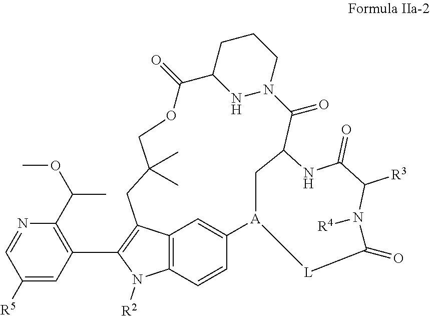

or pharmaceutically acceptable salt, an enantiomer, a stereoisomer, or a tautomer thereof, wherein
R⁵ is hydrogen, optionally substituted 3- to 10-membered heterocycloalkyl, —OR^{5a}, or optionally substituted C₁-C₆ heteroalkyl; and
R^{5a} is optionally substituted C₁-C₆ alkyl or optionally substituted 5- to 10-membered heteroaryl.

In some embodiments the disclosure features a compound of structural Formula IIIa-1:

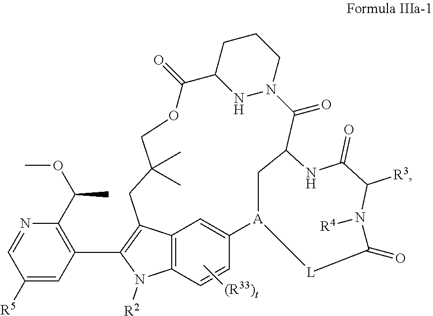

or pharmaceutically acceptable salt, an enantiomer, a stereoisomer, or a tautomer thereof.

In some embodiments, the disclosure features a compound of structural Formula IIIa-2:

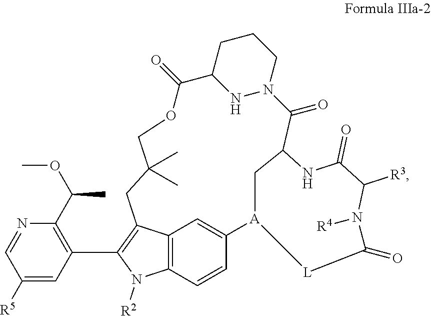

or pharmaceutically acceptable salt, an enantiomer, a stereoisomer, or a tautomer thereof.

In some embodiments, the compound has the structure of Formula IIIa-1:

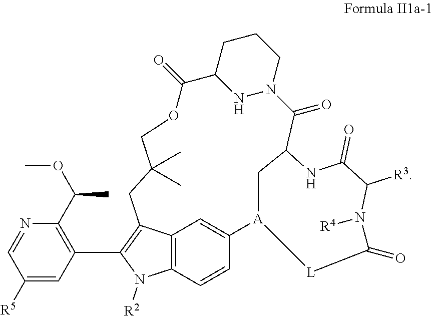

or pharmaceutically acceptable salt, an enantiomer, a stereoisomer, or a tautomer thereof.

In some embodiments, L has the structure of Formula III:

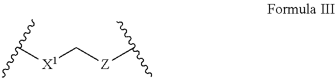

wherein X¹ is O or CH₂ and is attached to ring A; and
Z is optionally substituted 3- to 6-membered heterocycloalkylene, optionally substituted C₁-C₆ alkylene, or optionally substituted C₁-C₆ heteroalkylene.

In some embodiments, the compound has the structure of Formula Va-2:

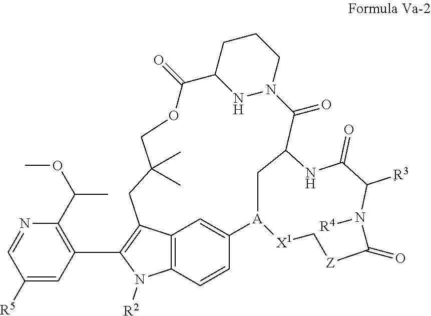

or pharmaceutically acceptable salt, an enantiomer, a stereoisomer, or a tautomer thereof, wherein
X¹ is O or CH₂; and
Z is optionally substituted 3- to 6-membered heterocycloalkylene, optionally substituted C₁-C₆ alkylene, or optionally substituted C₁-C₆ heteroalkylene.

In some embodiments, X¹ is O.

In some embodiments, Z is optionally substituted 3- to 6-membered heterocycloalkylene. In some embodiments, Z is optionally substituted 5-membered heterocycloalkylene. In some embodiments, Z is optionally substituted pyrollidine-diyl. In some embodiments, Z is optionally substituted pyrrolidinyl. In some embodiments, Z is optionally substituted pyrollidine-diyl.

In some embodiments, L has the structure of Formula IV:

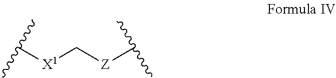

X¹ is O or CH₂ and is attached to ring A; and
Z is optionally substituted 3- to 6-membered heterocycloalkylene, optionally substituted C₁-C₆ alkylene, or optionally substituted C₁-C₆ heteroalkylene.

In some embodiments, L has the structure of Formula VI:

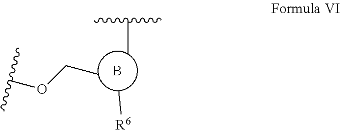

wherein B is an optionally substituted 3- to 6-membered heterocycloalkylene;
R⁶ is hydrogen, optionally substituted C₁-C₆ alkyl, optionally substituted C₁-C₆ heteroalkyl, optionally substituted optionally substituted 3- to 6-membered heterocyclyl, optionally substituted 3- to 6-membered cycloalkyl, optionally substituted 5- to 10-membered heteroaryl, optionally substituted C₆-C₁₀ aryl,

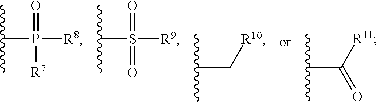

R⁷ and R⁸ are each, independently, H or optionally substituted C₁-C₆ alkyl;
R⁹ is optionally substituted C₁-C₆ alkyl, optionally substituted C₂-C₆ alkenyl, optionally substituted 3- to 6-membered cycloalkyl, or optionally substituted 3- to 6-membered heterocyclyl;
R¹⁰ is optionally substituted C₁-C₆ alkyl, optionally substituted C₂-C₆ alkenyl, optionally substituted C₁-C₆ heteroalkyl, optionally substituted C₃-C₁₀ cycloalkyl, optionally substituted 3- to 6-membered heterocyclyl, optionally substituted 5- to 10-membered heteroaryl, or optionally substituted C₆-C₁₀ aryl; and
R¹¹ is hydrogen, optionally substituted C₁-C₆ alkyl, optionally substituted C₁-C₆ heteroalkyl, optionally substituted C₂-C₆ alkenyl, optionally substituted C₂-C₆ heteroalkenyl, optionally substituted C₂-C₆ alkynyl, optionally substituted C₂-C₆ heteroalkynyl, optionally substituted C₃-C₁₀ cycloalkyl, optionally substituted 3- to 10-membered heterocyclyl, optionally substituted C₃-C₁₀ cycloalkenyl, optionally substituted 3- to 10-membered heterocycloalkenyl, optionally substituted C₆-C₁₀ aryl, or optionally substituted 5- to 10-membered heteroaryl.

In some embodiments, L has the structure of Formula VIa:

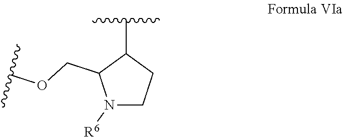

In some embodiments, the compound has the structure of Formula VIIa-2:

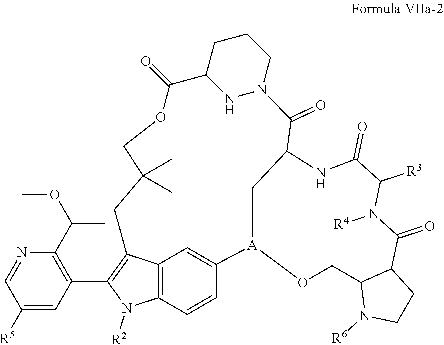

or pharmaceutically acceptable salt, an enantiomer, a stereoisomer, or a tautomer thereof, wherein
R⁶ is hydrogen, optionally substituted C₁-C₆ alkyl, optionally substituted C₁-C₆ heteroalkyl, optionally substituted 3- to 6-membered heterocyclyl, optionally substituted 3- to 6-membered cycloalkyl, optionally substituted 5- to 10-membered heteroaryl, optionally substituted C₆-C₁₀ aryl,

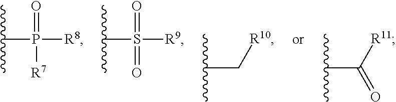

R⁷ and R⁸ are each, independently, H or optionally substituted C₁-C₆ alkyl;
R⁹ is optionally substituted C₁-C₆ alkyl, optionally substituted C₂-C₆ alkenyl, optionally substituted 3- to 6-membered cycloalkyl, or optionally substituted 3- to 6-membered heterocyclyl;
R¹⁰ is optionally substituted C₁-C₆ alkyl, optionally substituted C₂-C₆ alkenyl, optionally substituted C₁-C₆ heteroalkyl, optionally substituted C₃-C₁₀ cycloalkyl, optionally substituted 3- to 6-membered heterocyclyl, optionally substituted 5- to 10-membered heteroaryl, or optionally substituted C₆-C₁₀ aryl; and
R¹¹ is hydrogen, optionally substituted C₁-C₆ alkyl, optionally substituted C₁-C₆ heteroalkyl, optionally substituted C₂-C₆ alkenyl, optionally substituted C₂-C₆ heteroalkenyl, optionally substituted C₂-C₆ alkynyl, optionally substituted C₂-C₆ heteroalkynyl, optionally substituted C₃-C₁₀ cycloalkyl, optionally substituted 3- to 10-membered heterocyclyl, optionally substituted C₃-C₁₀ cycloalkenyl, optionally substituted 3- to 10-membered heterocycloalkenyl, optionally substituted C₆-C₁₀ aryl, or optionally substituted 5- to 10-membered heteroaryl.

In some embodiments, the compound has the structure of Formula VIIa or Formula VIIb:

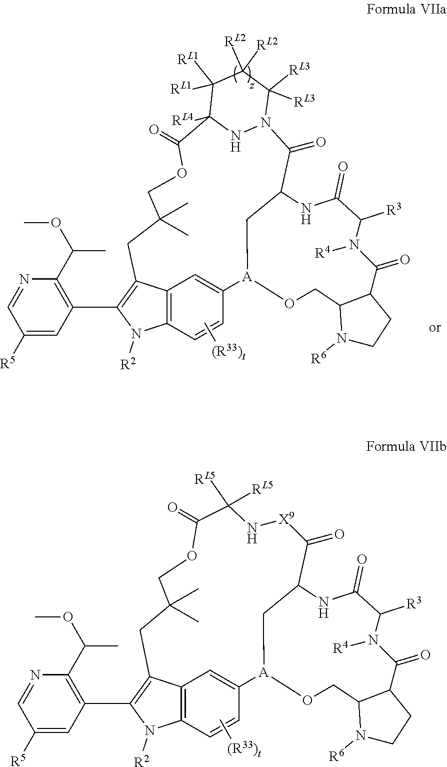

or pharmaceutically acceptable salt, an enantiomer, a stereoisomer, or a tautomer thereof, wherein
R⁶ is hydrogen, optionally substituted C₁-C₆ alkyl, optionally substituted C₁-C₆ heteroalkyl, optionally substituted 3- to 6-membered heterocyclyl, optionally substituted 3- to 6-membered cycloalkyl, optionally substituted 5- to 10-membered heteroaryl, optionally substituted C₆-C₁₀ aryl,

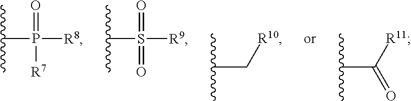

R⁷ and R⁸ are each, independently, H or optionally substituted C₁-C₆ alkyl;
R⁹ is optionally substituted C₁-C₆ alkyl, optionally substituted C₂-C₆ alkenyl, optionally substituted 3- to 6-membered cycloalkyl, or optionally substituted 3- to 6-membered heterocyclyl;
R¹⁰ is optionally substituted C₁-C₆ alkyl, optionally substituted C₂-C₆ alkenyl, optionally substituted C₁-C₆ heteroalkyl, optionally substituted C₃-C₁₀ cycloalkyl, optionally substituted 3- to 6-membered heterocyclyl, optionally substituted 5- to 10-membered heteroaryl, or optionally substituted C₆-C₁₀ aryl; and
R¹¹ is hydrogen, optionally substituted C₁-C₆ alkyl, optionally substituted C₁-C₆ heteroalkyl, optionally substituted C₂-C₆ alkenyl, optionally substituted C₂-C₆ heteroalkenyl, optionally substituted C₂-C₆ alkynyl, optionally substituted C₂-C₆ heteroalkynyl, optionally substituted C₃-C₁₀ cycloalkyl, optionally substituted 3- to 10-membered heterocyclyl, optionally substituted C₃-C₁₀ cycloalkenyl, optionally substituted 3- to 10-membered heterocycloalkenyl, optionally substituted C₆-C₁₀ aryl, or optionally substituted 5- to 10-membered heteroaryl.

In some embodiments, the compound has the structure of Formula VIIa-1:

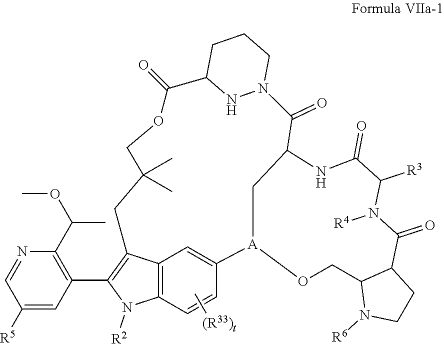

or pharmaceutically acceptable salt, an enantiomer, a stereoisomer, or a tautomer thereof, wherein
R⁶ is hydrogen, optionally substituted C₁-C₆ alkyl, optionally substituted C₁-C₆ heteroalkyl, optionally substituted 3- to 6-membered heterocyclyl, optionally substituted 3- to 6-membered cycloalkyl, optionally substituted 5- to 10-membered heteroaryl, optionally substituted C₆-C₁₀ aryl,

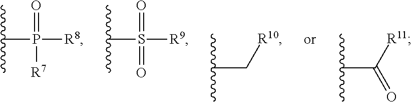

R⁷ and R⁸ are each, independently, H or optionally substituted C₁-C₆ alkyl;
R⁹ is optionally substituted C₁-C₆ alkyl, optionally substituted C₂-C₆ alkenyl, optionally substituted 3- to 6-membered cycloalkyl, or optionally substituted 3- to 6-membered heterocyclyl;
R¹⁰ is optionally substituted C₁-C₆ alkyl, optionally substituted C₂-C₆ alkenyl, optionally substituted C₁-C₆ heteroalkyl, optionally substituted C₃-C₁₀ cycloalkyl, optionally substituted 3- to 6-membered heterocyclyl, optionally substituted 5- to 10-membered heteroaryl, or optionally substituted C₆-C₁₀ aryl; and
R¹¹ is hydrogen, optionally substituted C₁-C₆ alkyl, optionally substituted C₁-C₆ heteroalkyl, optionally substituted C₂-C₆ alkenyl, optionally substituted C₂-C₆ heteroalkenyl, optionally substituted C₂-C₆ alkynyl, optionally substituted C₂-C₆ heteroalkynyl, optionally substituted C₃-C₁₀ cycloalkyl, optionally substituted 3- to 10-membered heterocyclyl, optionally substituted C₃-C₁₀ cycloalkenyl, optionally substituted 3- to 10-membered heterocycloalkenyl, optionally substituted C₆-C₁₀ aryl, or optionally substituted 5- to 10-membered heteroaryl.

In some embodiments, the compound has the structure of Formula VIIa-2:

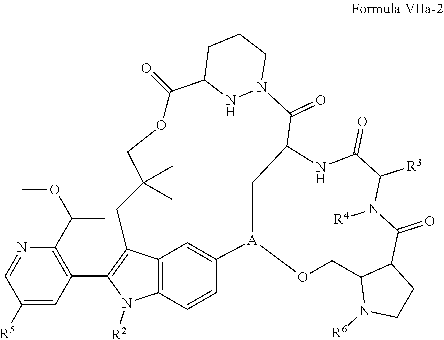

or pharmaceutically acceptable salt, an enantiomer, a stereoisomer, or a tautomer thereof, wherein
R⁶ is hydrogen, optionally substituted C₁-C₆ alkyl, optionally substituted C₁-C₆ heteroalkyl, optionally substituted 3- to 6-membered heterocyclyl, optionally substituted 3- to 6-membered cycloalkyl, optionally substituted 5- to 10-membered heteroaryl, optionally substituted C₆-C₁₀ aryl,

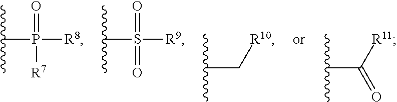

R⁷ and R⁸ are each, independently, H or optionally substituted C₁-C₆ alkyl;
R⁹ is optionally substituted C₁-C₆ alkyl, optionally substituted C₂-C₆ alkenyl, optionally substituted 3- to 6-membered cycloalkyl, or optionally substituted 3- to 6-membered heterocyclyl;
R¹⁰ is optionally substituted C₁-C₆ alkyl, optionally substituted C₂-C₆ alkenyl, optionally substituted C₁-C₆ heteroalkyl, optionally substituted C₃-C₁₀ cycloalkyl, optionally substituted 3- to 6-membered heterocyclyl, optionally substituted 5- to 10-membered heteroaryl, or optionally substituted C₆-C₁₀ aryl; and
R¹¹ is hydrogen, optionally substituted C₁-C₆ alkyl, optionally substituted C₁-C₆ heteroalkyl, optionally substituted C₂-C₆ alkenyl, optionally substituted C₂-C₆ heteroalkenyl, optionally substituted C₂-C₆ alkynyl, optionally substituted C₂-C₆ heteroalkynyl, optionally substituted C₃-C₁₀ cycloalkyl, optionally substituted 3- to 10-membered heterocyclyl, optionally substituted C₃-C₁₀ cycloalkenyl, optionally substituted 3- to 10-membered heterocycloalkenyl, optionally substituted C₆-C₁₀ aryl, or optionally substituted 5- to 10-membered heteroaryl. In some embodiments, R⁶ is

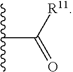

In some embodiments, R¹¹ is optionally substituted C₁-C₆ alkyl. In some embodiments, R¹¹ is optionally substituted C₂-C₆ alkenyl. In some embodiments, R¹¹ is optionally substituted C₂-C₆ alkynyl. In some embodiments, R¹¹ is optionally substituted C₁-C₆ heteroalkyl. In some embodiments, R¹¹ is optionally substituted C₂-C₆ heteroalkenyl. In some embodiments, R¹¹ is optionally substituted C₂-C₆ heteroalkynyl. In some embodiments, R¹¹ is optionally substituted C₃-C₁₀ cycloalkenyl. In some embodiments, R¹¹ is hydrogen. In some embodiments, R¹¹ is optionally substituted C₃-C₁₀ cycloalkyl. In some embodiments, R¹¹ is optionally substituted 3- to 10-membered heterocyclyl.

In some embodiments, R⁶ is

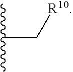

In some embodiments, R¹⁰ is optionally substituted 5- to 10-membered heteroaryl. In some embodiments, R¹⁰ is optionally substituted 3- to 10-membered heterocyclyl.

In some embodiments, R⁶ is optionally substituted 3- to 6-membered heterocyclyl.

In some embodiments, A is optionally substituted 3- to 6-membered heterocycloalkylene, optionally substituted 6-membered arylene, or optionally substituted 5- to 10-membered heteroarylene. In some embodiments, A is optionally substituted 6-membered arylene. In some embodiments, A is:

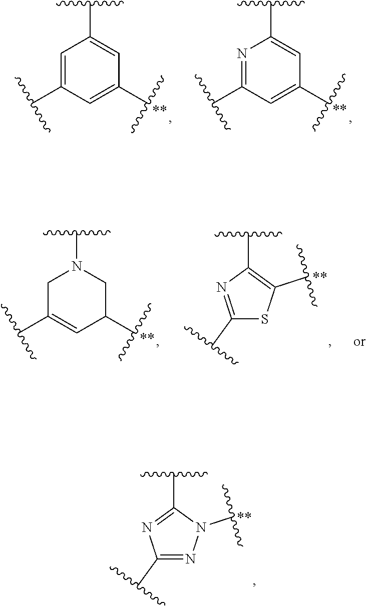 [SMILES: *c1cc(*)cc(*)c1 ; *c1cc(*)nc(*)c1 ; *C1=CC(*)CN(*)C1 ; *c1nc(*)c(*)s1 ; *c1nc(*)n(*)n1]

wherein

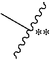

represents the portion of the molecule bound to the linker.

In some embodiments, A is

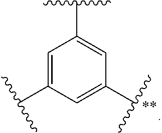 [SMILES: *c1cc(*)cc(*)c1]

In some embodiments, R² is C₁-C₃ alkyl or C₁-C₃ haloalkyl. In some embodiments, R² is

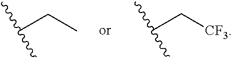

In some embodiments, R² is

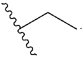 [SMILES: *CC]

In some embodiments, R⁴ is optionally substituted C₁-C₆ alkyl. In some embodiments, R⁴ is methyl.

In some embodiments, R³ is optionally substituted C₁-C₆ alkyl. In some embodiments, R³ is optionally substituted 3- to 6-membered cycloalkyl. In some embodiments, R³ is

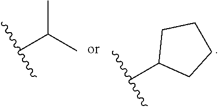

In some embodiments, R⁵ is hydrogen. In some embodiments, R⁵ is optionally substituted 3- to 10-membered heterocycloalkyl.

In some embodiments, R⁵ is

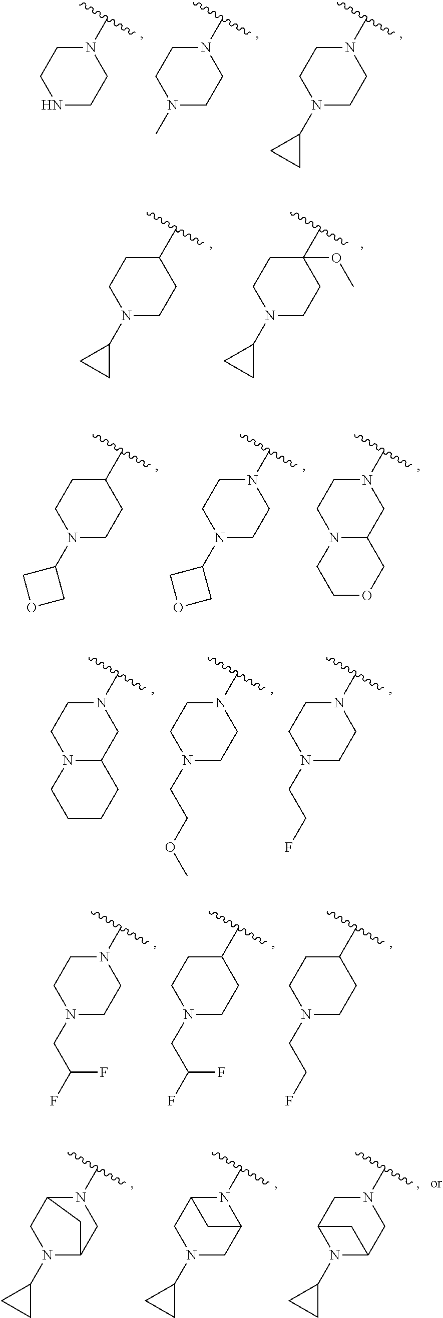

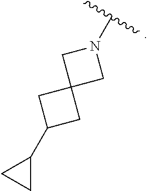 [SMILES: *N1CC2(CC(C3CC3)C2)C1]

In some embodiments, R⁵ is —OR⁵a. In some embodiments, R⁵ is

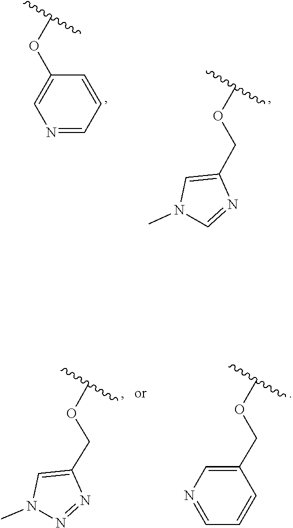

In any embodiment herein, a compound of the present invention may be modified with a substituent as found in any one or more of the following applications, incorporated herein by reference in their entireties: WO 2024/060966, WO 2024/017859, WO 2024/008834, WO 2024/008610, WO 2023/232776, WO 2023/208005, WO 2023/086341, WO 2023/025832, WO 2023/015559, CN 117720556, CN 117720555, CN 117720554, CN 117534687, CN 117534685 and CN 117534684.

In some embodiments, a compound of the present invention is selected from Tables 1 and 2, or a pharmaceutically acceptable salt or stereoisomer thereof. In some embodiments, a compound of the present invention is selected from Tables 1 and 2, or a pharmaceutically acceptable salt or atropisomer thereof.

TABLE 1
Certain Compounds of the Present Invention

| Ex# | Structure |
|---|---|
| A1 | 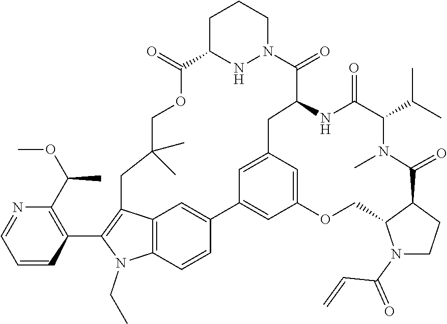 |
| A2 | 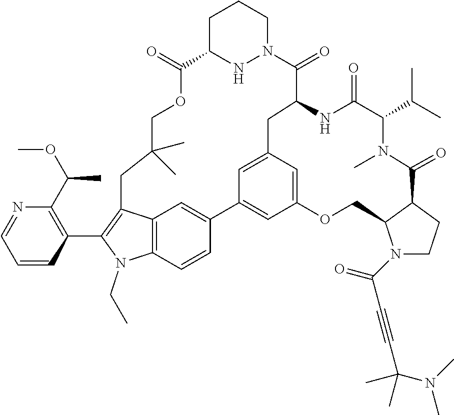 |
| A3 | 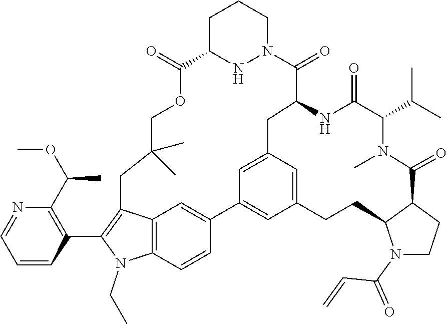 |
| A4 | 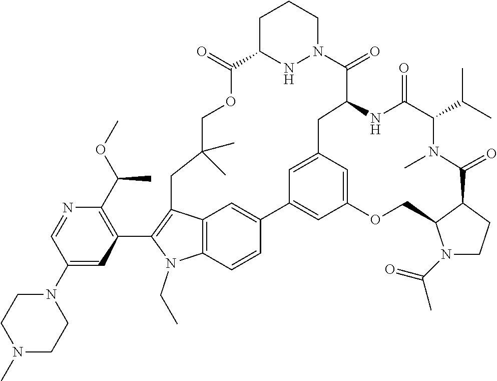 |
| A5 | 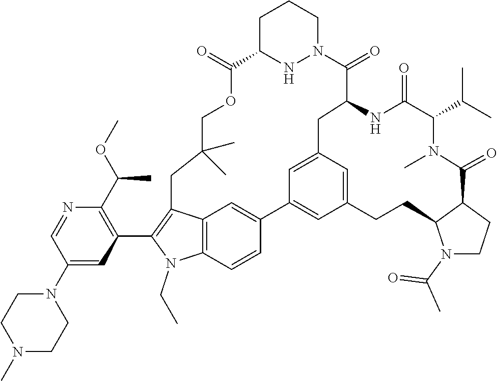 |
| A6 | 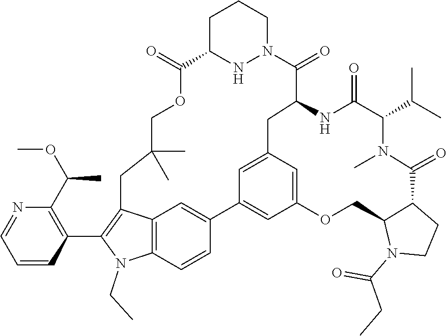 |
| A7 | 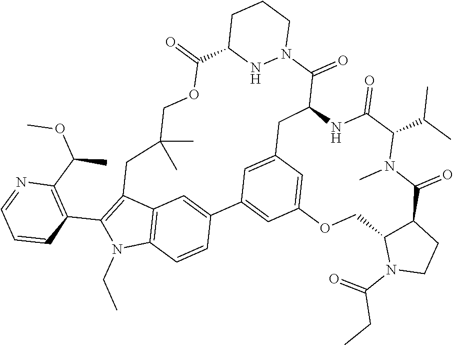 |
| A8 | 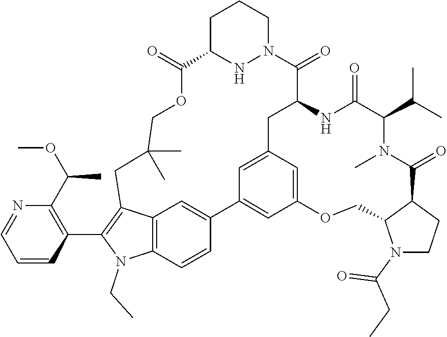 |
| A9 | 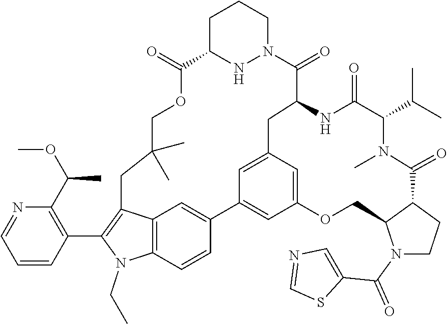 [SMILES: CCn1c(-c2cccnc2[C@H](C)OC)c2c3cc(ccc31)-c1cc3cc(c1)OC[C@H]1[C@@H](CCN1C(=O)c1cncs1)C(=O)N(C)[C@@H](C(C)C)C(=O)N[C@@H](C3)C(=O)N1CCC[C@H](N1)C(=O)OCC(C)(C)C2] |
| A10 | 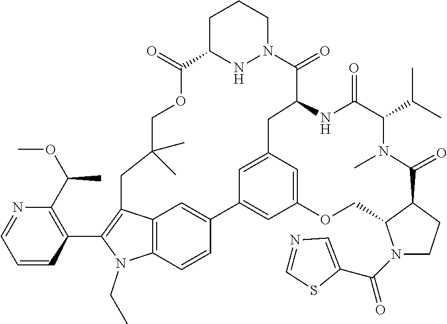 |
| A11 | 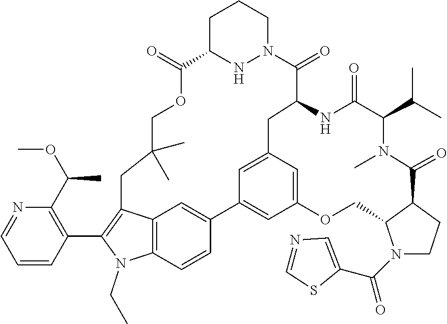 [SMILES: CCn1c(-c2cccnc2[C@H](C)OC)c2c3cc(ccc31)-c1cc3cc(c1)OC[C@@H]1[C@H](CCN1C(=O)c1cncs1)C(=O)N(C)[C@H](C(C)C)C(=O)N[C@@H](C3)C(=O)N1CCC[C@H](N1)C(=O)OCC(C)(C)C2] |
| A12 | 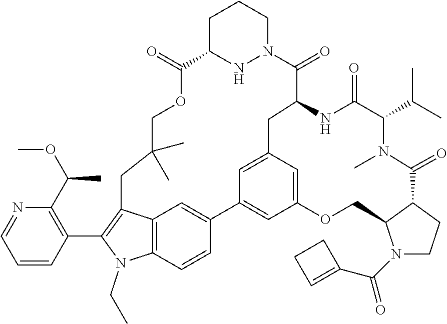 [SMILES: CCn1c(-c2cccnc2[C@H](C)OC)c2c3cc(ccc31)-c1cc3cc(c1)OC[C@H]1[C@@H](CCN1C(=O)C1=CCC1)C(=O)N(C)[C@@H](C(C)C)C(=O)N[C@@H](C3)C(=O)N1CCC[C@H](N1)C(=O)OCC(C)(C)C2] |
| A13 | 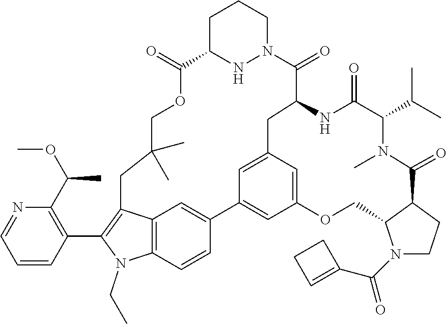 [SMILES: CCn1c(-c2cccnc2[C@H](C)OC)c2c3cc(ccc31)-c1cc3cc(c1)OC[C@@H]1[C@H](CCN1C(=O)C1=CCC1)C(=O)N(C)[C@@H](C(C)C)C(=O)N[C@@H](C3)C(=O)N1CCC[C@H](N1)C(=O)OCC(C)(C)C2] |
| A14 | 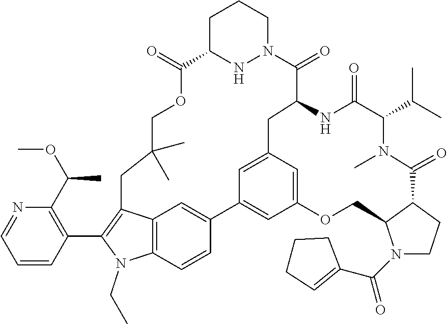 [SMILES: CCn1c(-c2cccnc2[C@H](C)OC)c2c3cc(ccc31)-c1cc3cc(c1)OC[C@H]1[C@@H](CCN1C(=O)C1=CCCC1)C(=O)N(C)[C@@H](C(C)C)C(=O)N[C@@H](C3)C(=O)N1CCC[C@H](N1)C(=O)OCC(C)(C)C2] |
| A15 | 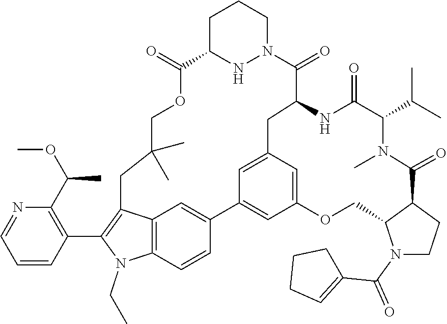 [SMILES: CCn1c(-c2cccnc2[C@H](C)OC)c2c3cc(ccc31)-c1cc3cc(c1)OC[C@@H]1[C@H](CCN1C(=O)C1=CCCC1)C(=O)N(C)[C@@H](C(C)C)C(=O)N[C@@H](C3)C(=O)N1CCC[C@H](N1)C(=O)OCC(C)(C)C2] |
| A16 | 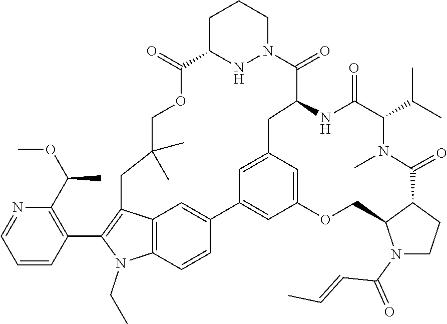 [SMILES: C/C=C/C(=O)N1CC[C@H]2C(=O)N(C)[C@@H](C(C)C)C(=O)N[C@H]3Cc4cc(cc(c4)-c4ccc5c(c4)c(c(-c4cccnc4[C@H](C)OC)n5CC)CC(C)(C)COC(=O)[C@@H]4CCCN(N4)C3=O)OC[C@@H]21] |
| A17 | 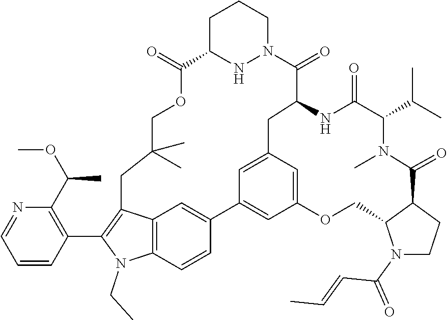 [SMILES: C/C=C/C(=O)N1CC[C@@H]2C(=O)N(C)[C@@H](C(C)C)C(=O)N[C@H]3Cc4cc(cc(c4)-c4ccc5c(c4)c(c(-c4cccnc4[C@H](C)OC)n5CC)CC(C)(C)COC(=O)[C@@H]4CCCN(N4)C3=O)OC[C@H]21] |
| A18 | 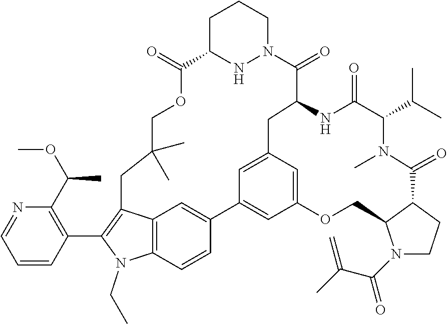 [SMILES: C=C(C)C(=O)N1CC[C@H]2C(=O)N(C)[C@@H](C(C)C)C(=O)N[C@H]3Cc4cc(cc(c4)-c4ccc5c(c4)c(c(-c4cccnc4[C@H](C)OC)n5CC)CC(C)(C)COC(=O)[C@@H]4CCCN(N4)C3=O)OC[C@@H]21] |
| A19 | 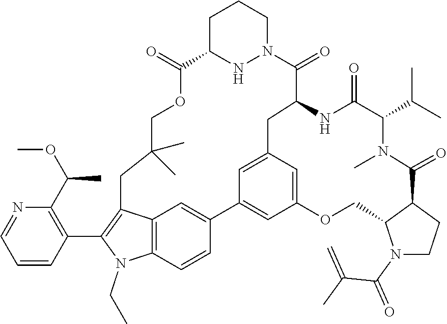 [SMILES: C=C(C)C(=O)N1CC[C@@H]2C(=O)N(C)[C@@H](C(C)C)C(=O)N[C@H]3Cc4cc(cc(c4)-c4ccc5c(c4)c(c(-c4cccnc4[C@H](C)OC)n5CC)CC(C)(C)COC(=O)[C@@H]4CCCN(N4)C3=O)OC[C@H]21] |
| A20 | 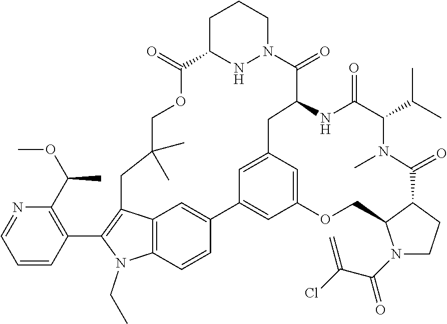 [SMILES: C=C(Cl)C(=O)N1CC[C@H]2C(=O)N(C)[C@@H](C(C)C)C(=O)N[C@H]3Cc4cc(cc(c4)-c4ccc5c(c4)c(c(-c4cccnc4[C@H](C)OC)n5CC)CC(C)(C)COC(=O)[C@@H]4CCCN(N4)C3=O)OC[C@@H]21] |
| A21 | 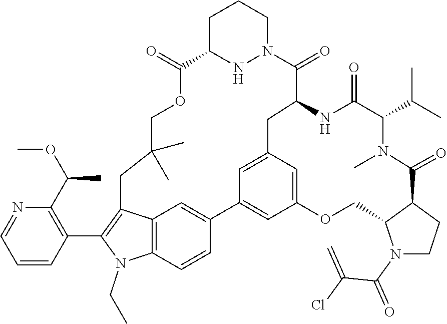 [SMILES: C=C(Cl)C(=O)N1CC[C@@H]2C(=O)N(C)[C@@H](C(C)C)C(=O)N[C@H]3Cc4cc(cc(c4)-c4ccc5c(c4)c(c(-c4cccnc4[C@H](C)OC)n5CC)CC(C)(C)COC(=O)[C@@H]4CCCN(N4)C3=O)OC[C@H]21] |
| A22 | 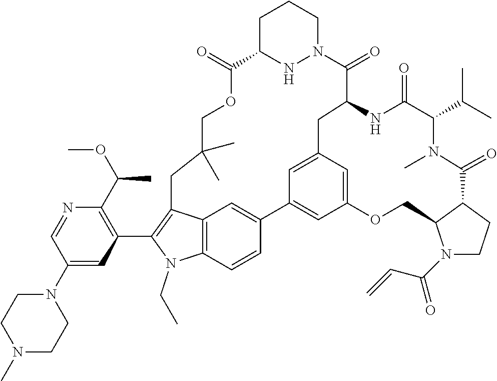 |
| A23 | 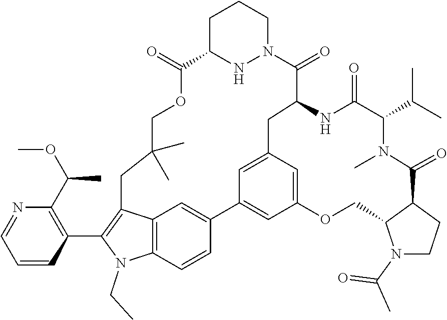 |
| A24 | 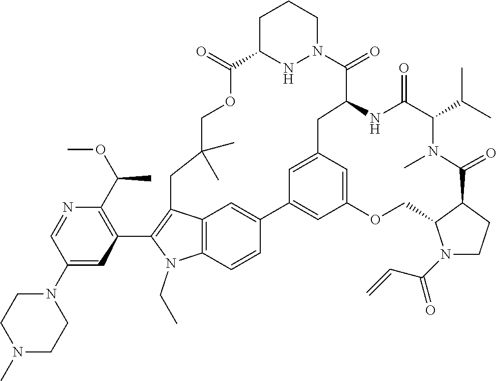 |
| A25 | 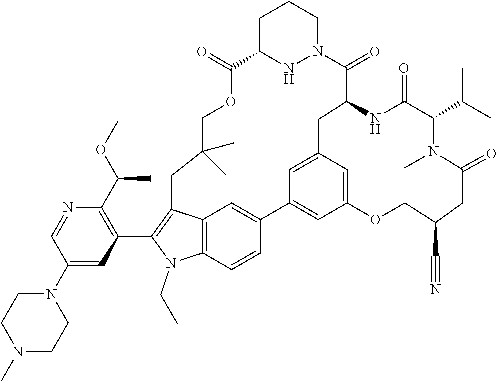 |
| A26 | 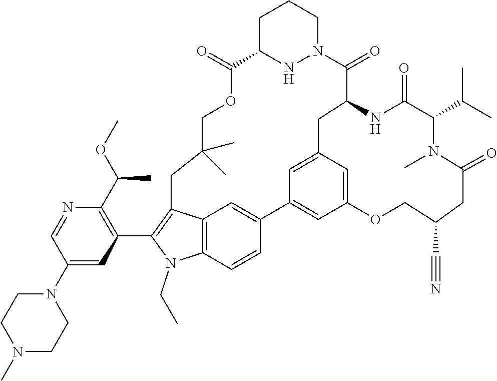 |
| A27 | 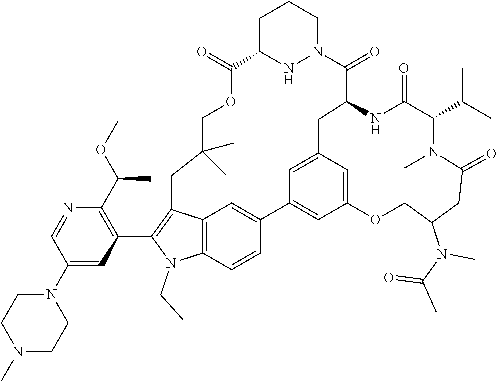 |
| A28 | 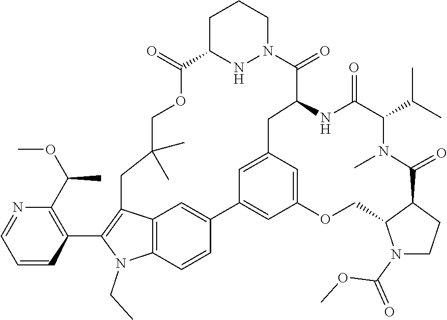 |
| A29 | 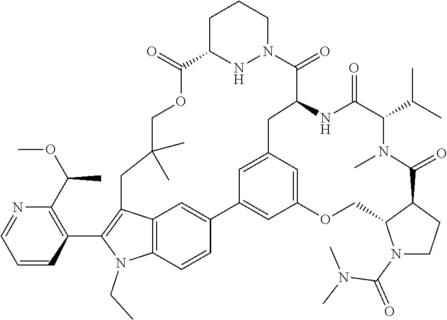 |
| A30 | 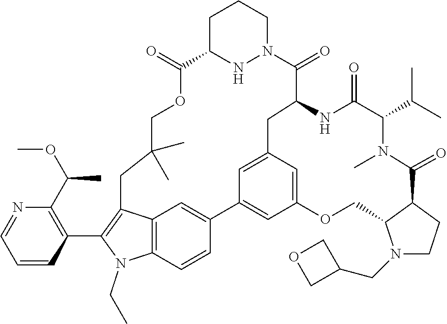 |
| A31 | 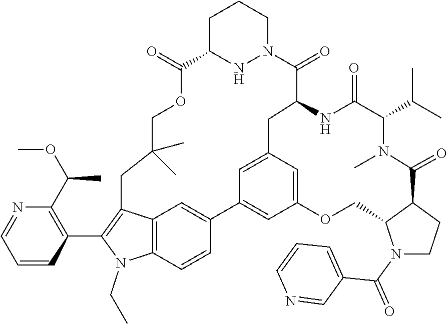 |
| A32 | 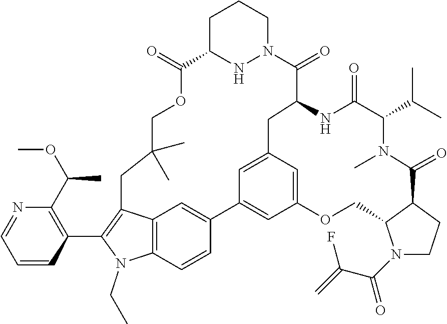 |
| A33 | 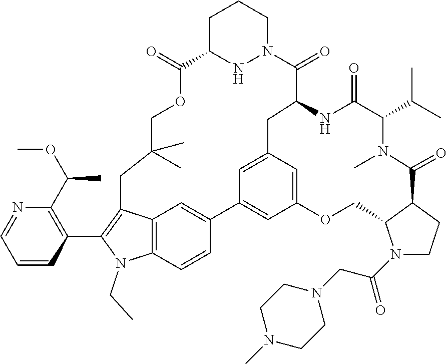 |
| A34 | 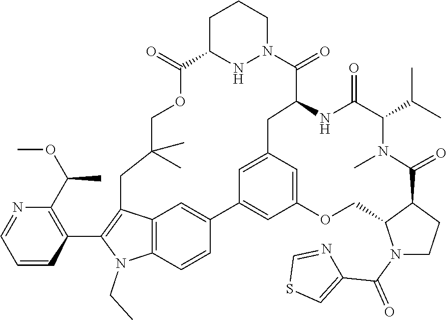 |
| A35 | 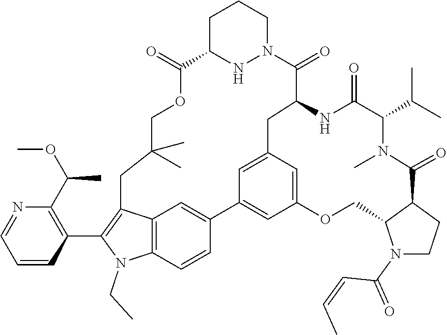 |
| A36 | 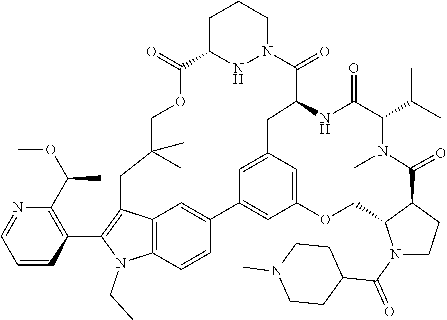 |
| A37 | 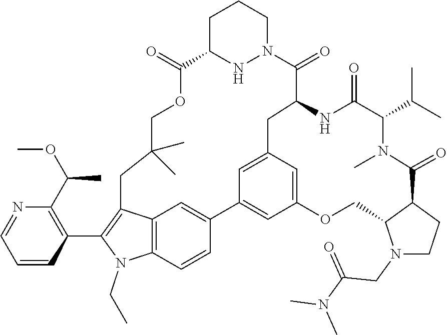 |
| A38 | 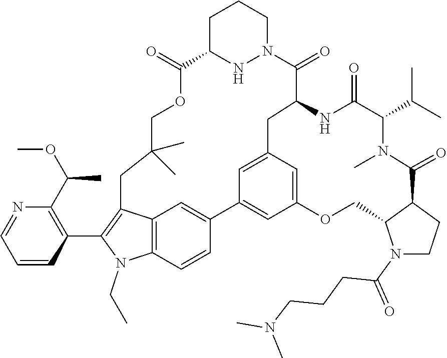 |
| A39 | 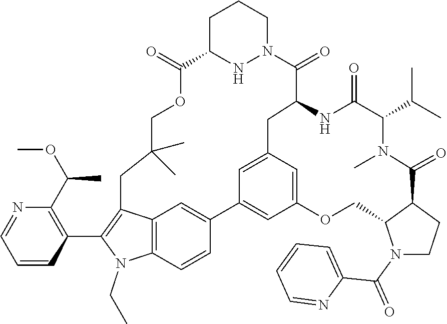 |
| A40 | 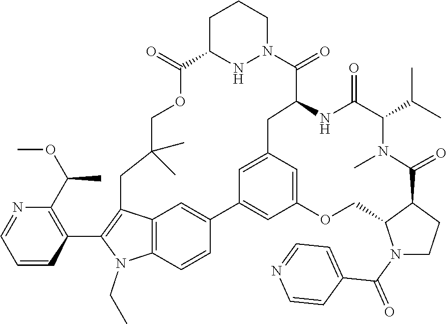 |
| A41 | 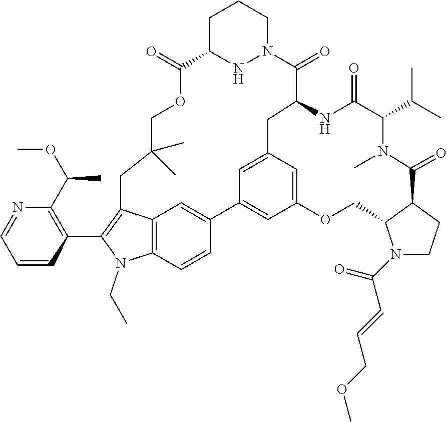 |
| A42 | 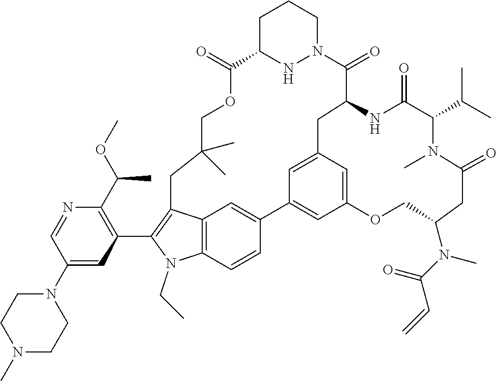 |
| A43 | 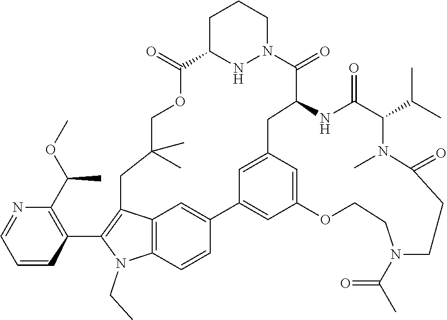 |
| A44 | 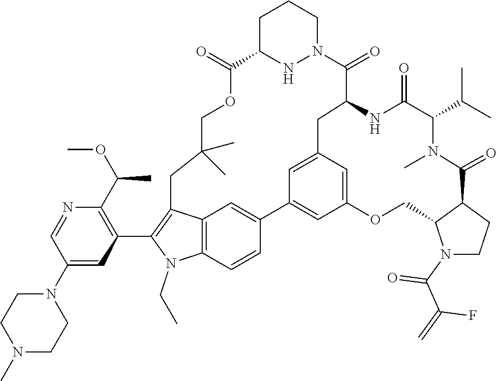 |
| A45 | 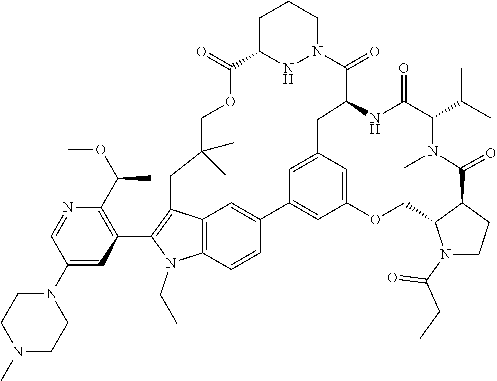 |
| A46 | 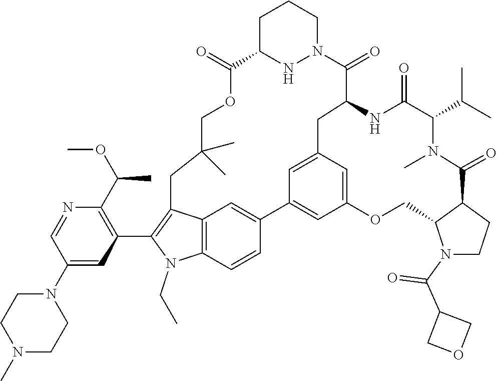 |
| A47 | 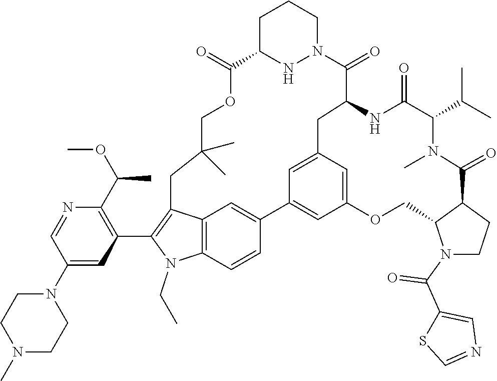 |
| A48 | 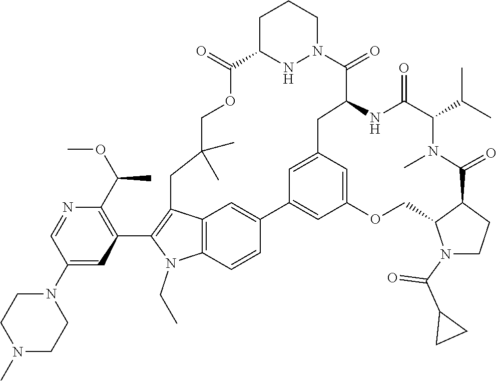 |
| A49 | 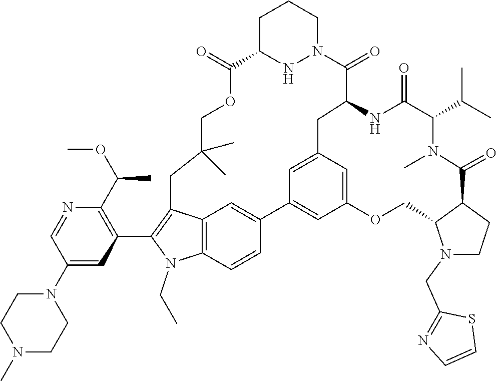 |
| A50 | 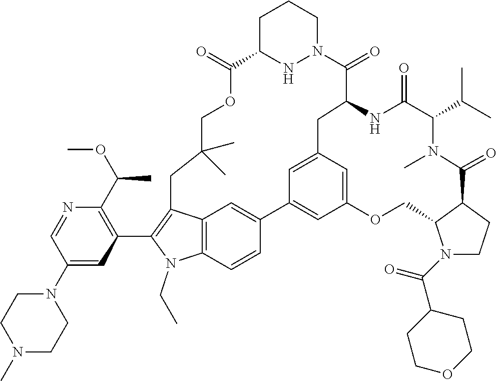 |
| A51 | 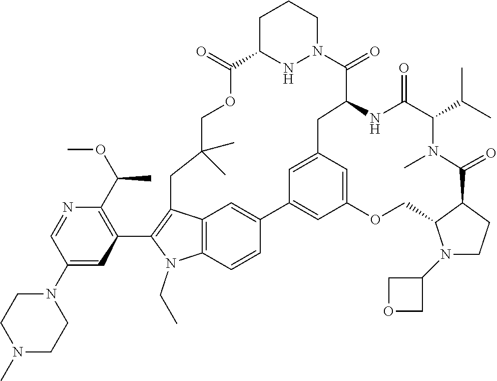 |
| A52 | 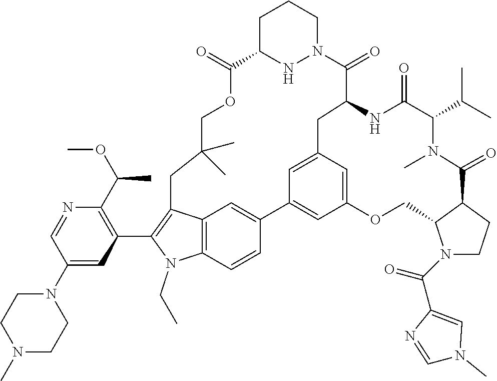 |
| A53 | 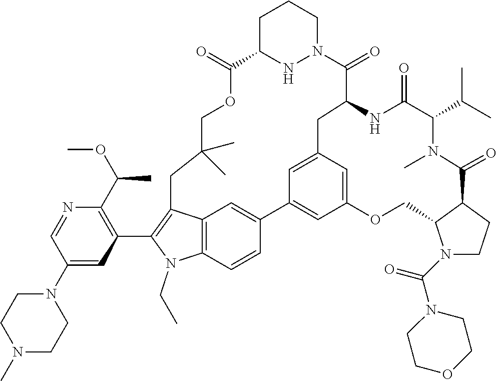 |
| A54 | 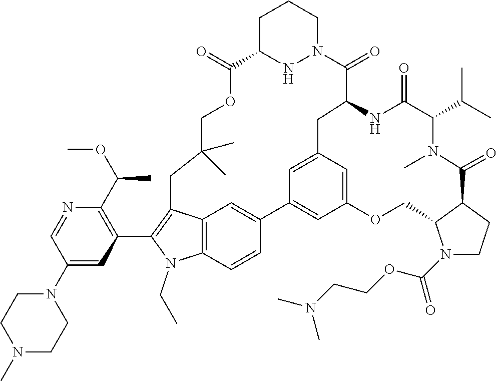 |
| A55 | 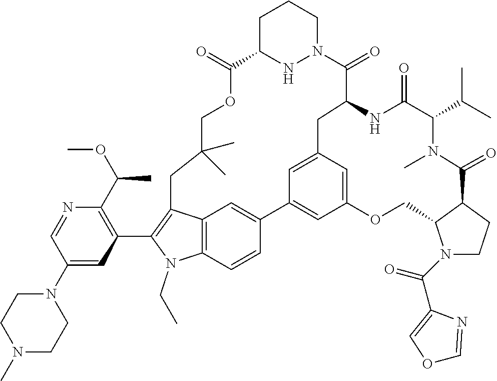 |
| A56 | 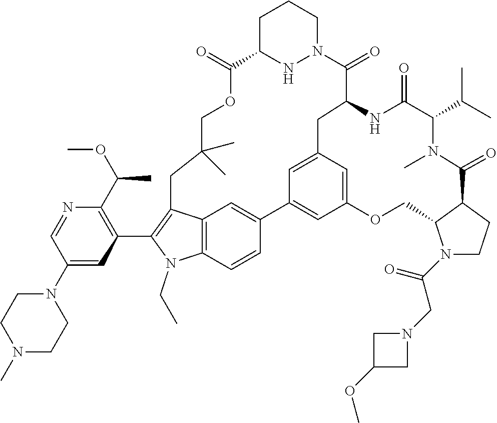 |
| A57 | 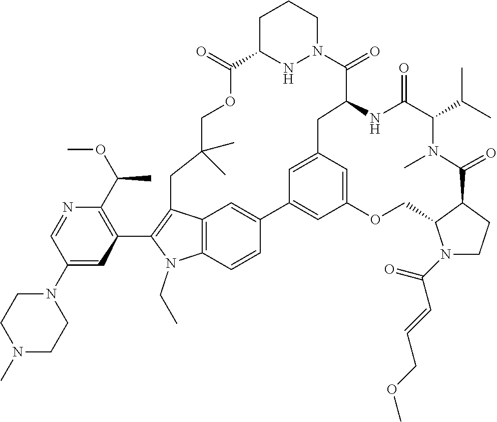 |
| A58 | 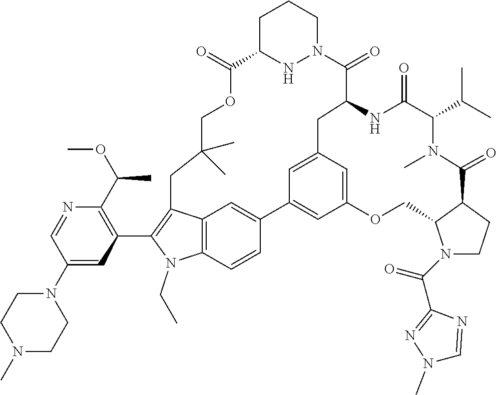 |
| A59 | 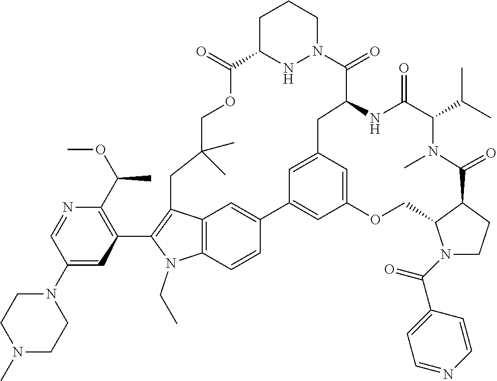 |
| A60 | 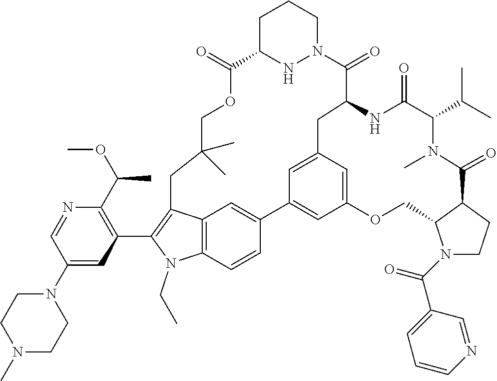 |
| A61 | 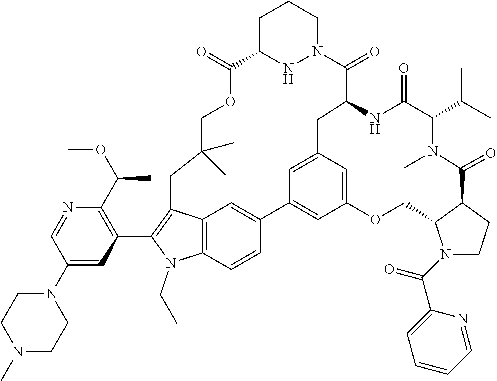 |
| A62 | 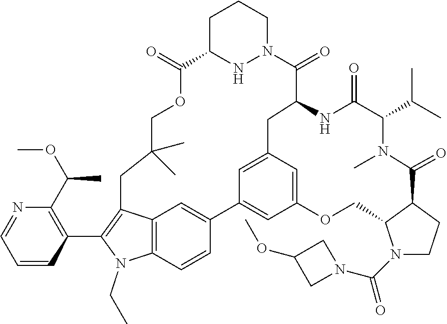 |
| A63 | 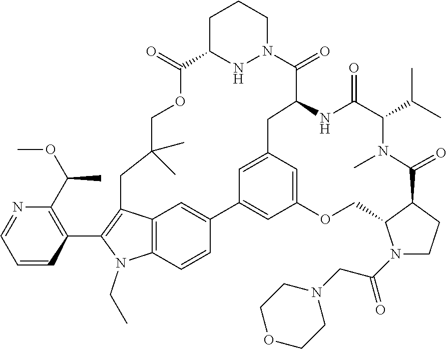 |
| A64 | 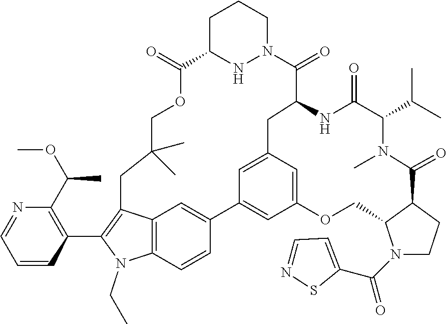 |
| A65 | 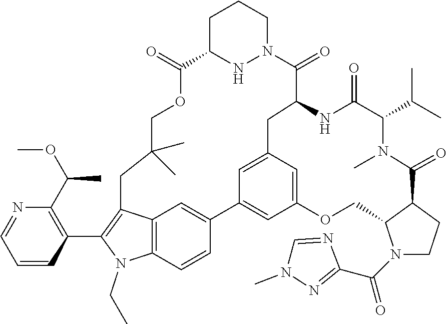 |
| A66 | 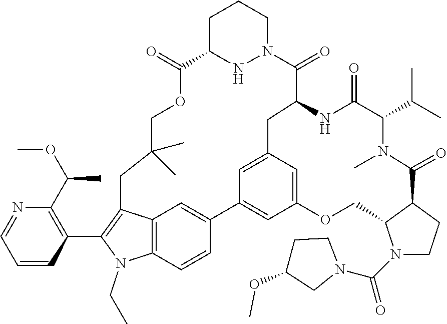 |
| A67 | 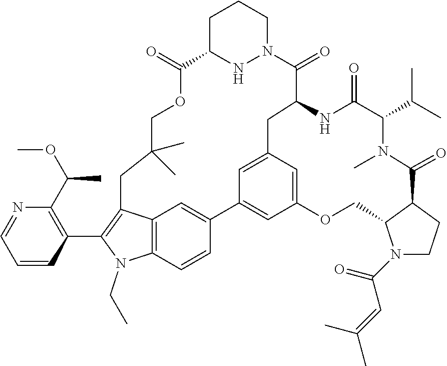 |
| A68 | 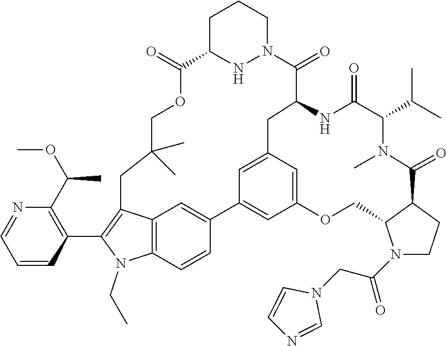 |
| A69 | 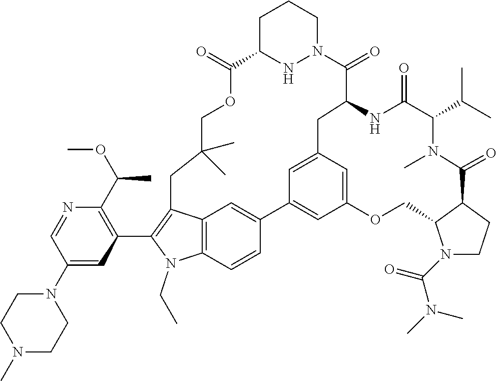 |
| A70 | 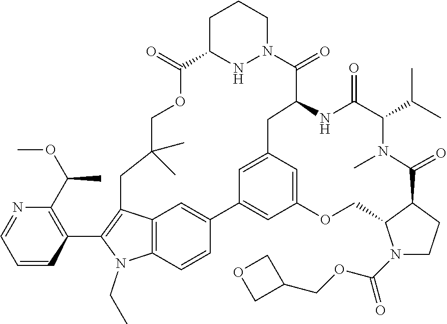 |
| A71 | 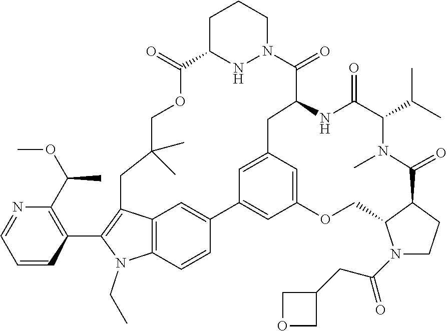 |
| A72 | 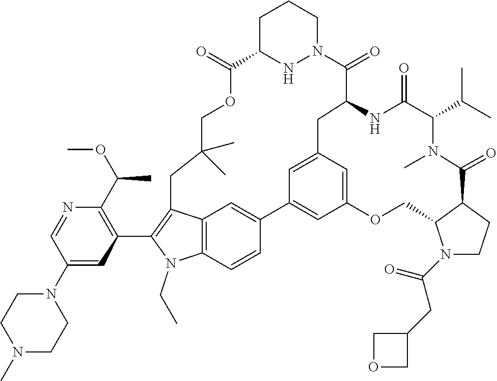 |
| A73 | 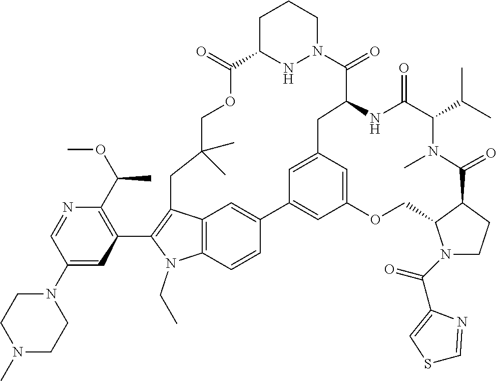 |
| A74 | 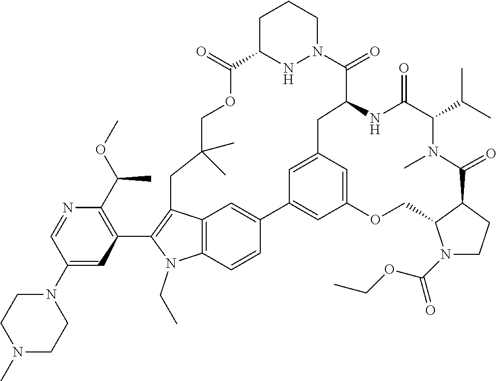 |
| A75 | 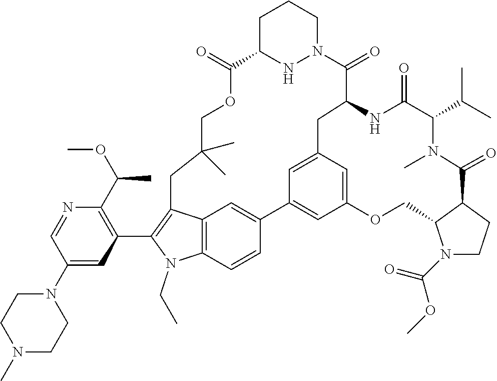 |
| A76 | 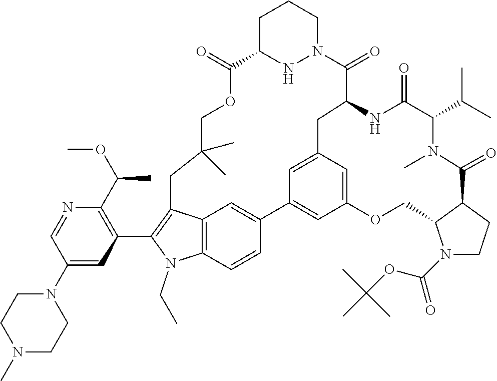 |
| A77 | 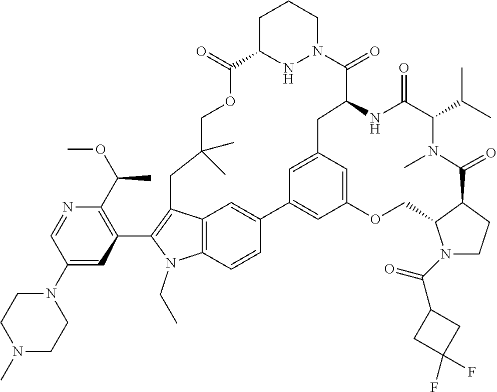 |
| A78 | 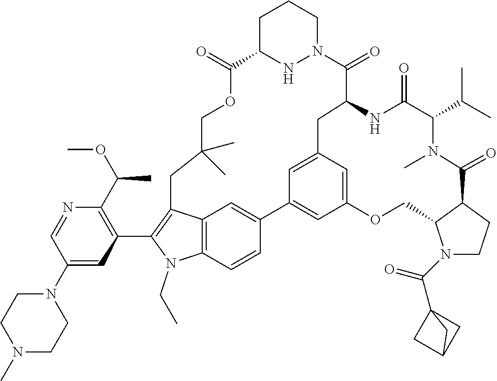 |
| A79 | 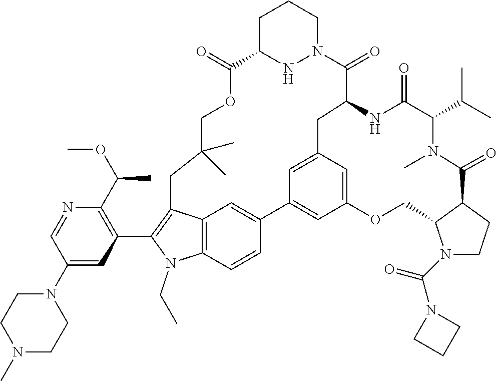 |
| A80 | 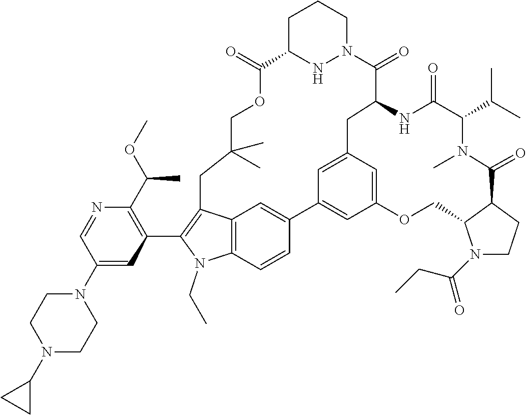 |
| A81 | 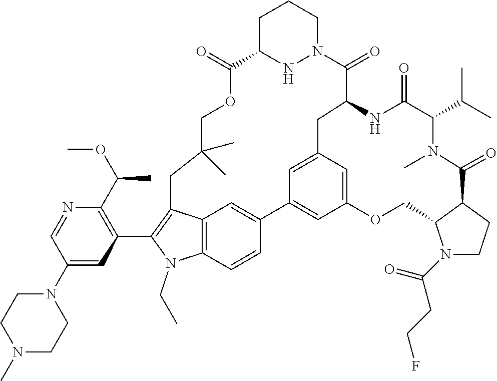 |
| A82 | 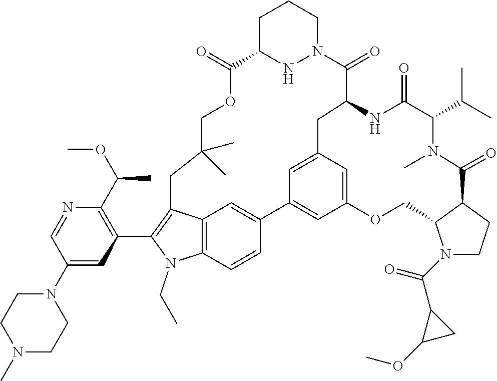 |
| A83 | 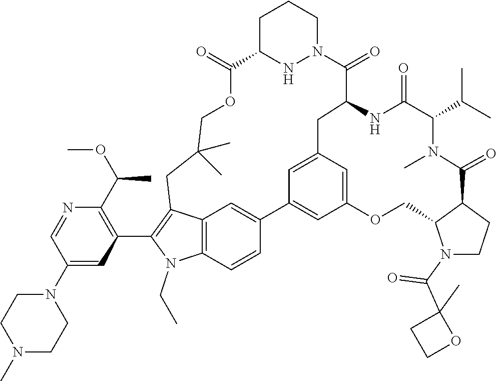 |
| A84 | 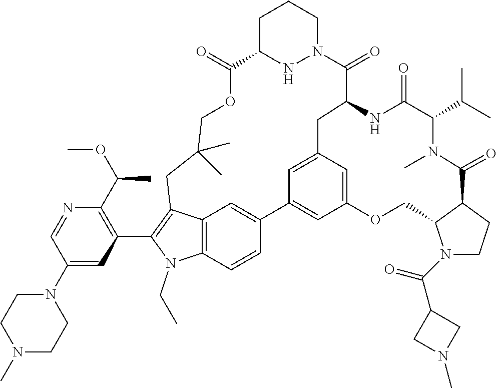 |
| A85 | 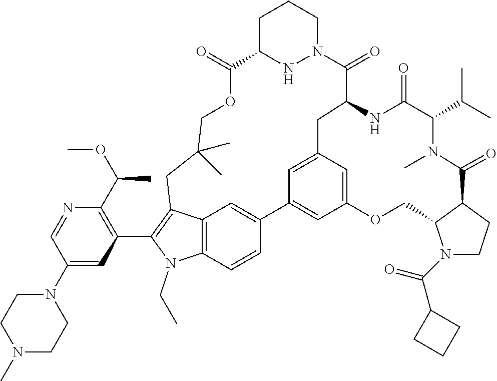 |
| A86 | 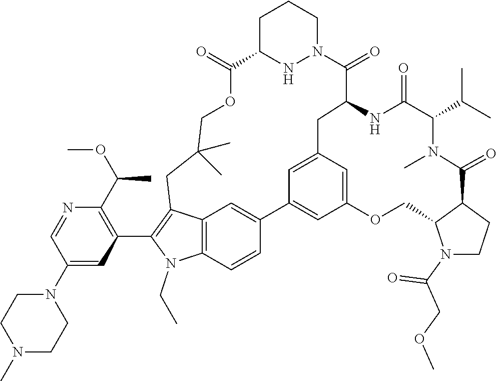 |
| A87 | 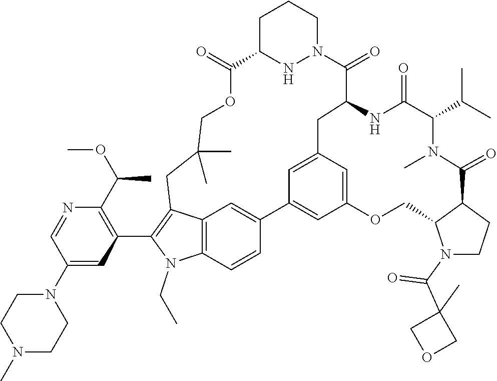 |
| A88 | 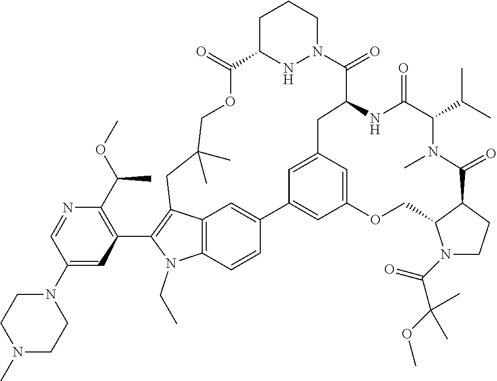 |
| A89 | 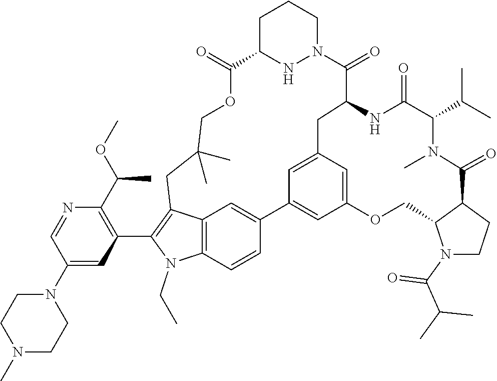 |
| A90 | 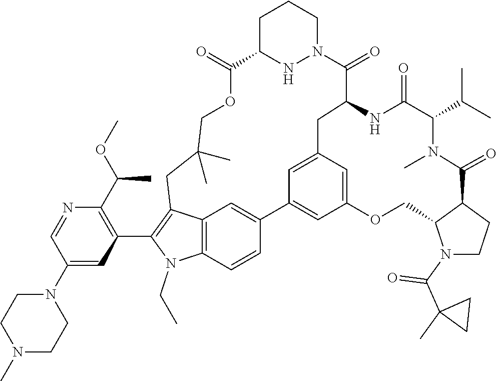 |
| A91 | 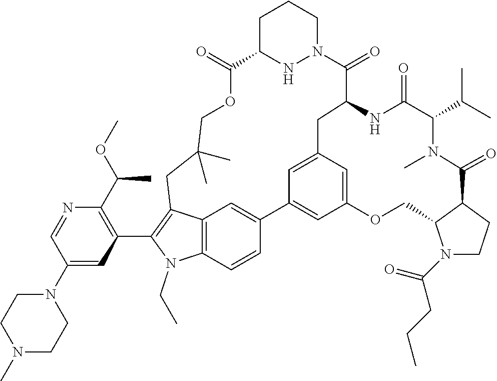 |
| A92 | 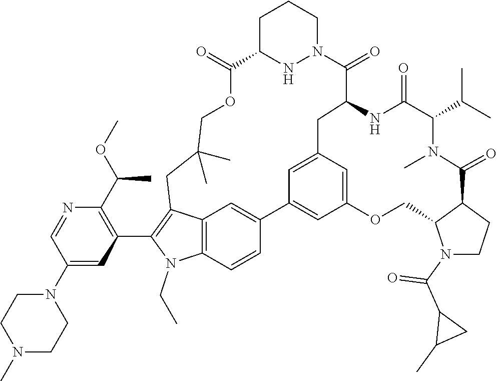 |
| A93 | 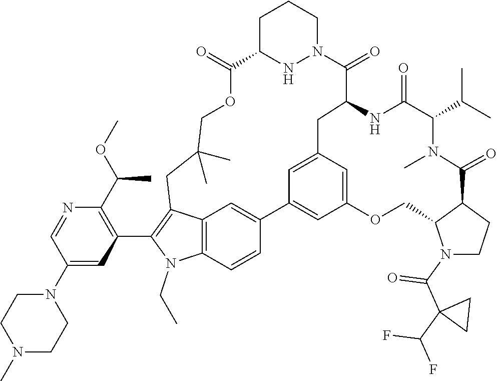 |
| A94 | 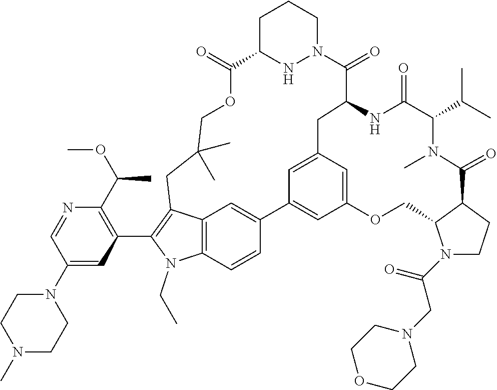 |
| A95 | 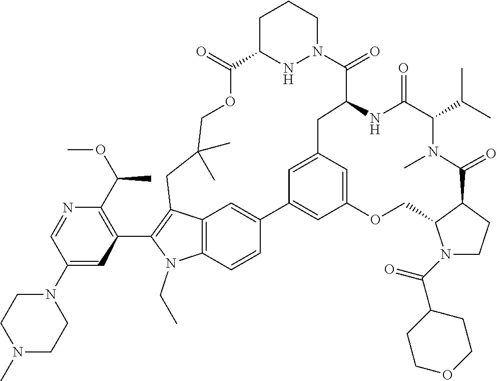 |
| A96 | 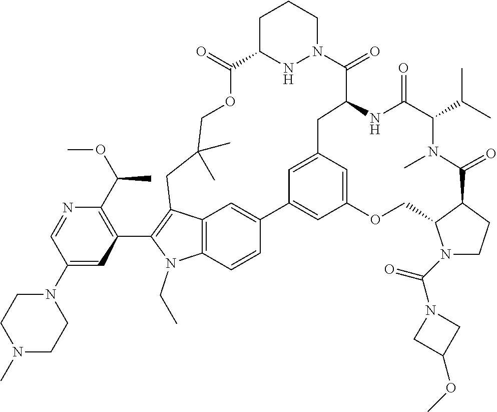 |
| A97 | 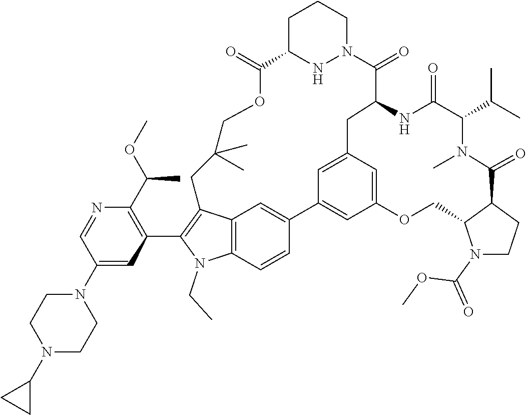 |
| A98 | 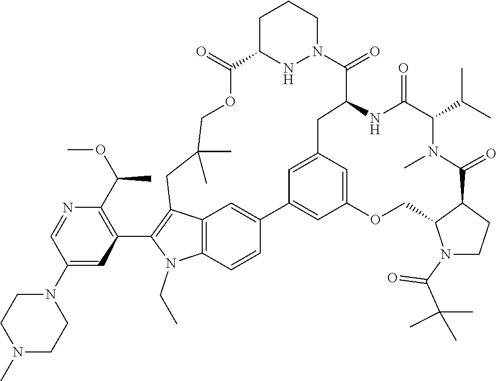 |
| A99 | 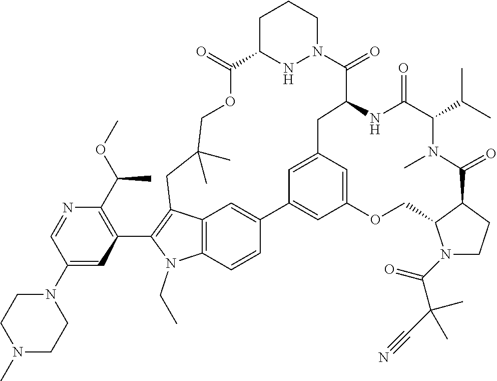 |
| A100 | 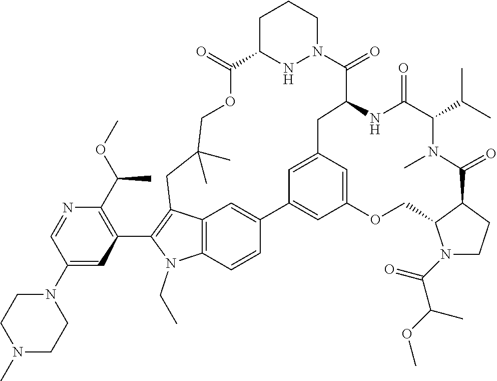 |
| A101 | 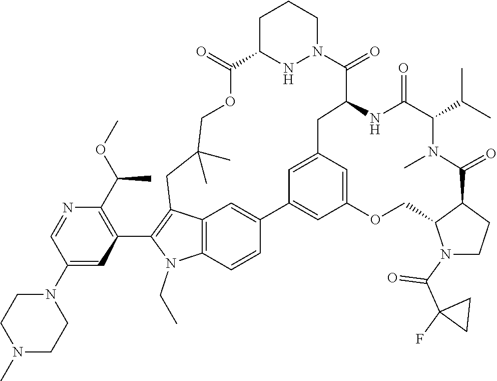 |
| A102 | 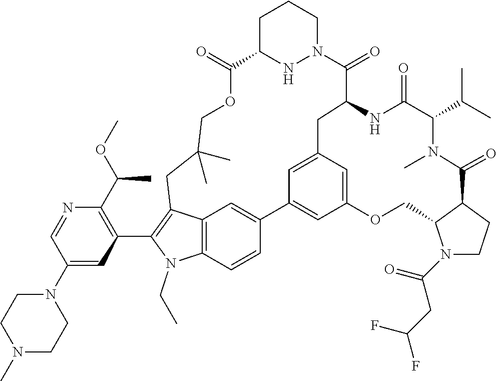 |
| A103 | 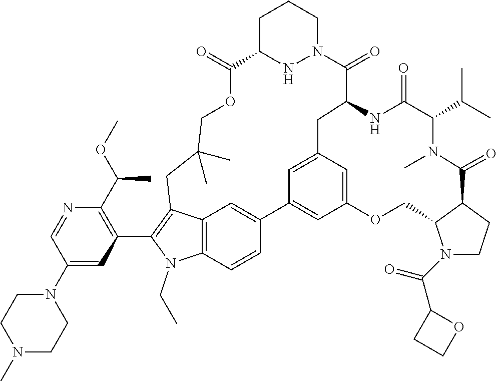 |
| A104 | 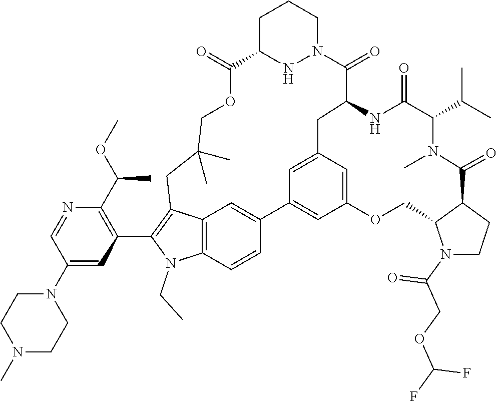 |
| A105 | 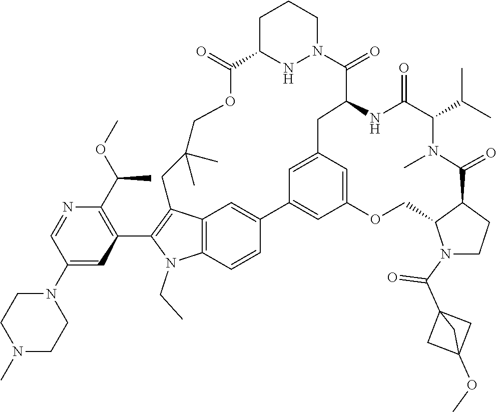 |
| A106 | 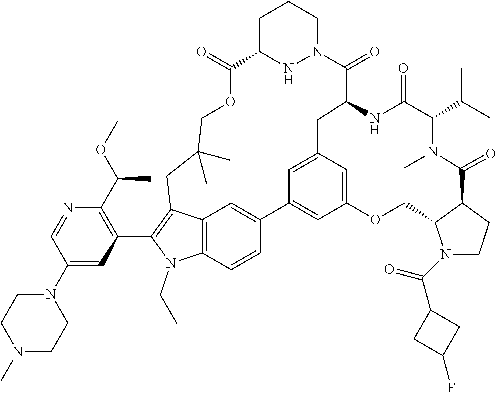 |
| A107 | 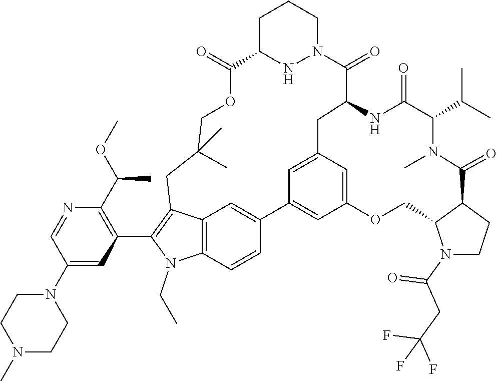 |
| A108 | 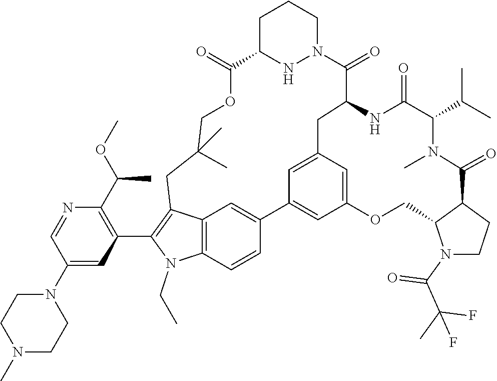 |
| A109 | 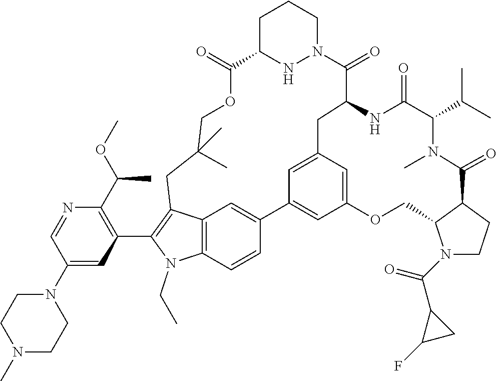 |
| A110 | 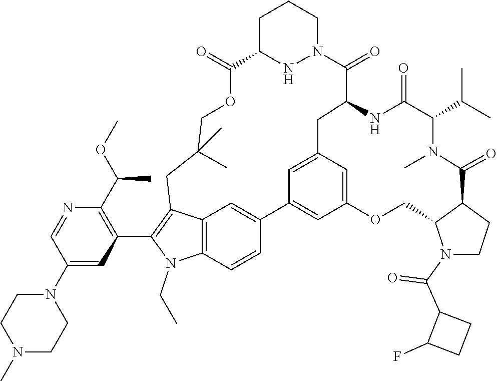 |
| A111 | 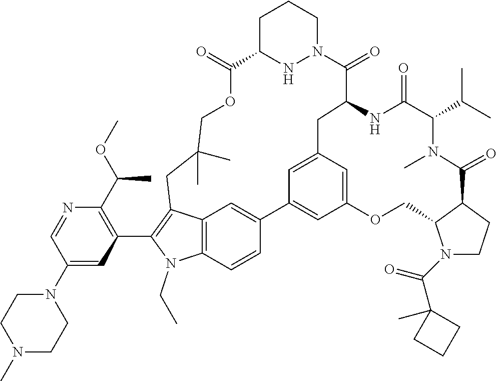 |
| A112 | 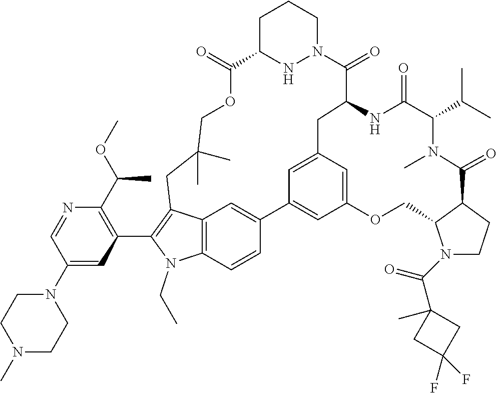 |
| A113 | 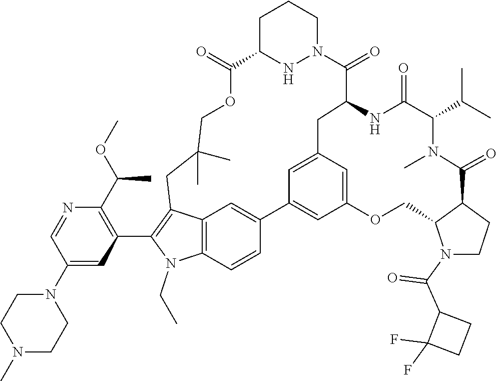 |
| A114 | 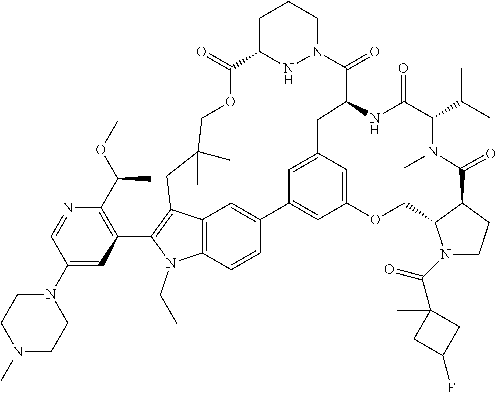 |
| A115 | 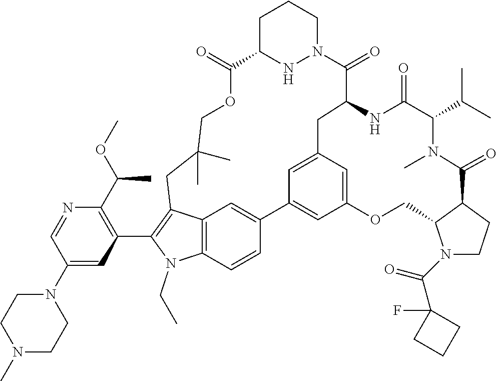 |
| A116 | 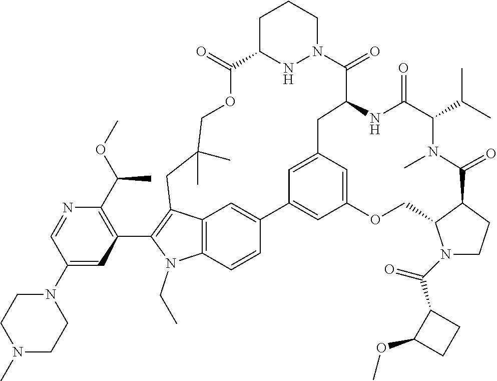 |
| A117 | 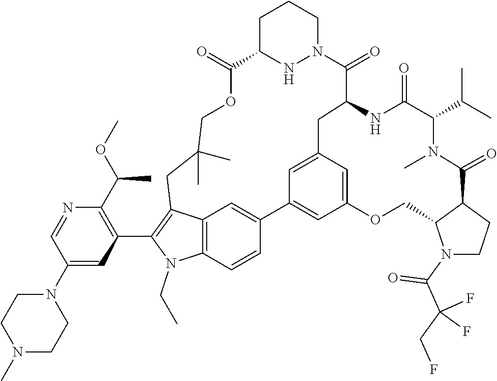 |
| A118 | 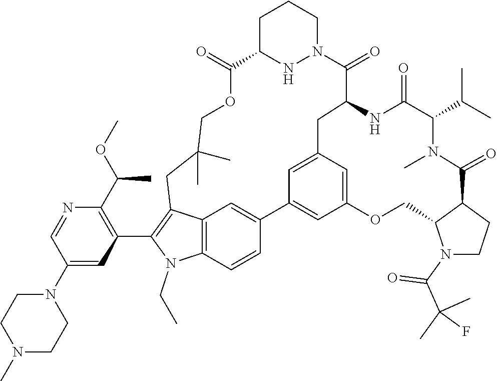 |
| A119 | 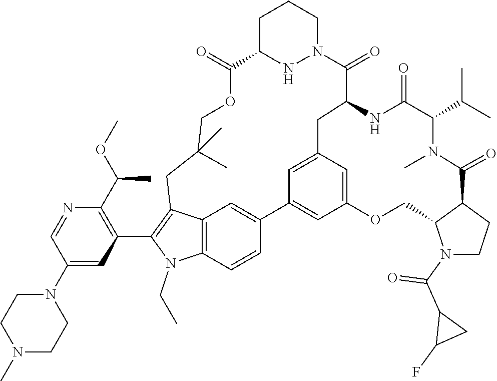 |
| A120 | 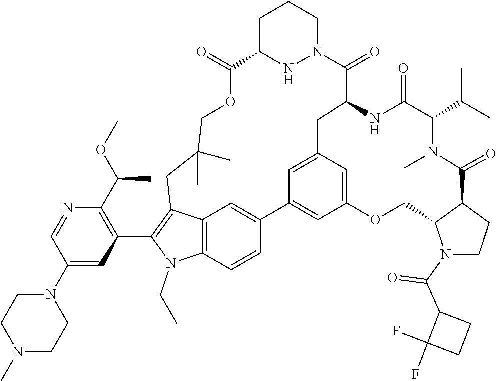 |
| A121 | 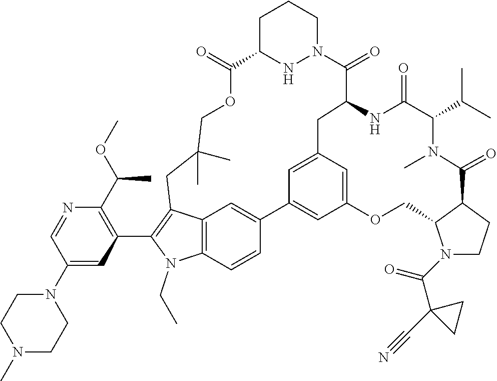 |
| A122 | 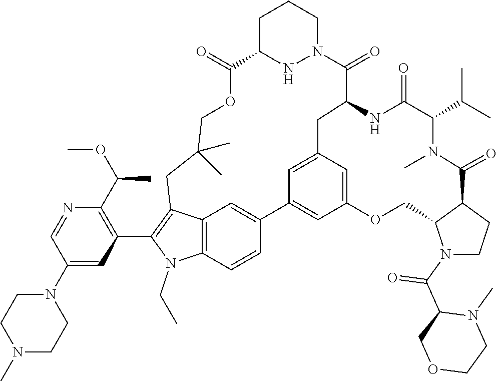 |
| A123 | 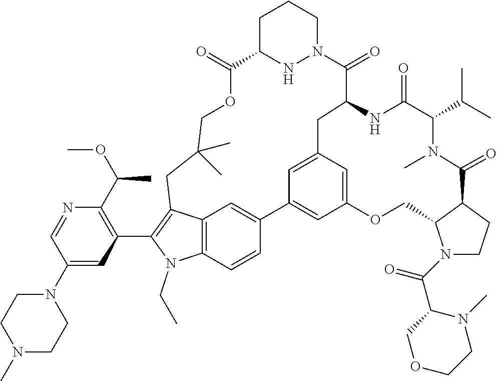 |
| A124 | 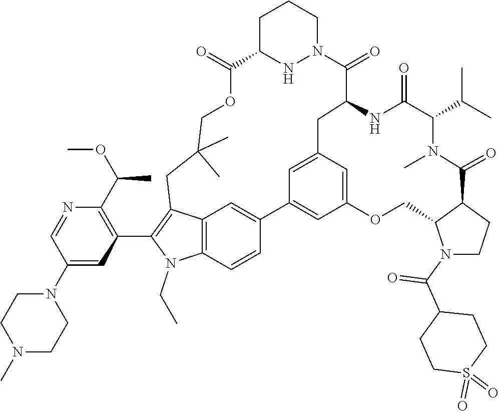 |
| A125 | 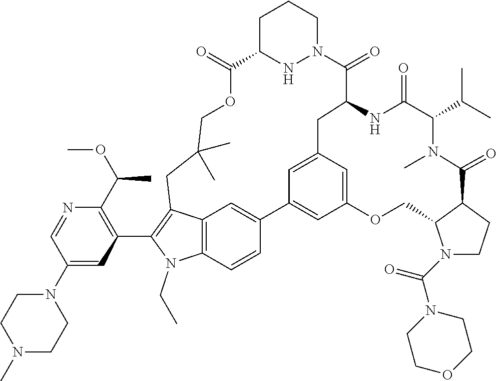 |
| A126 | 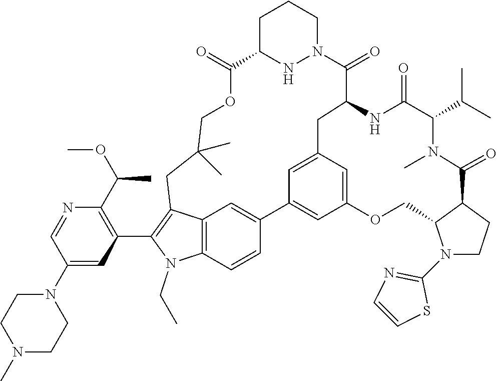 |
| A127 | 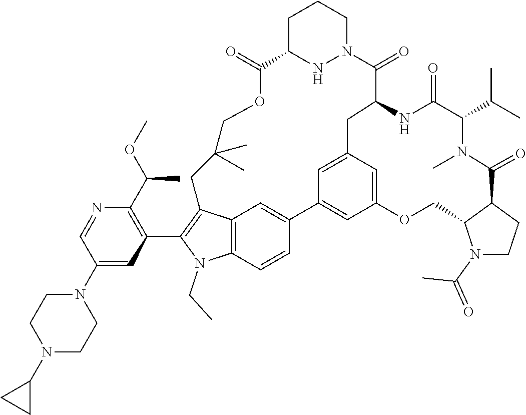 |
| A128 | 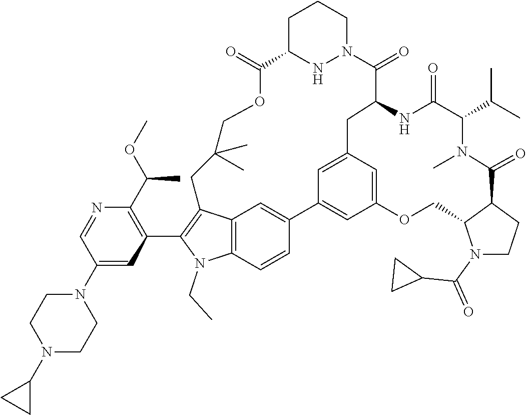 |
| A129 | 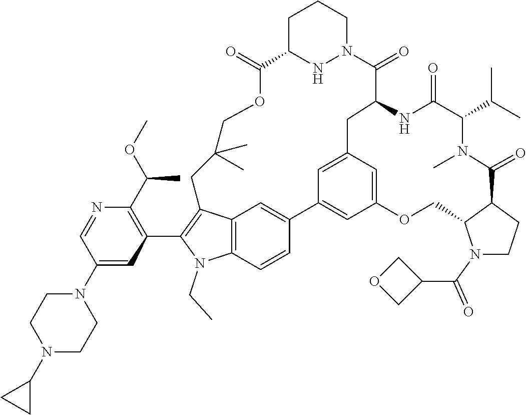 |
| A130 | 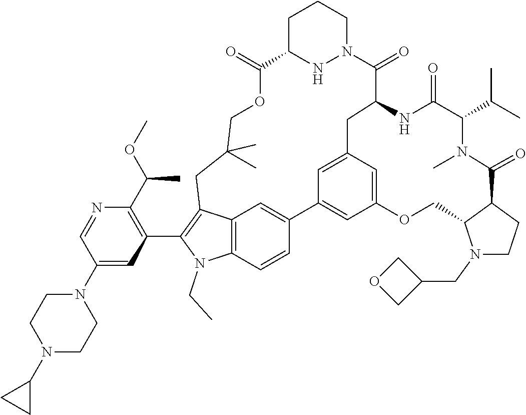 |
| A131 | 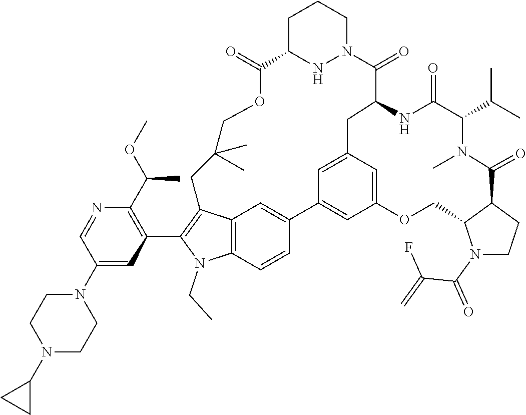 |
| A132 | 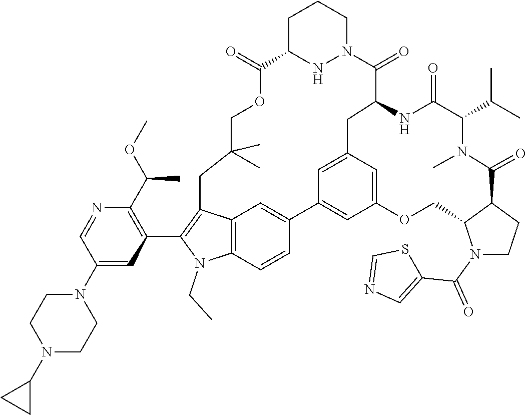 |
| A133 | 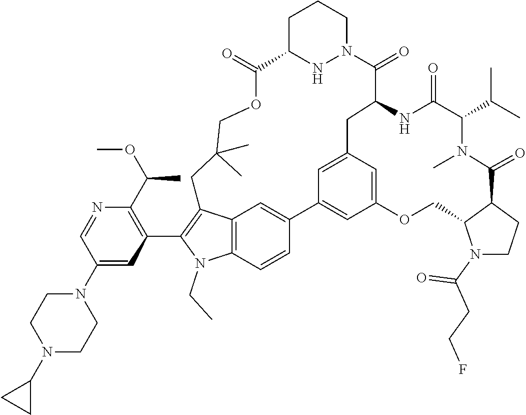 |
| A134 | 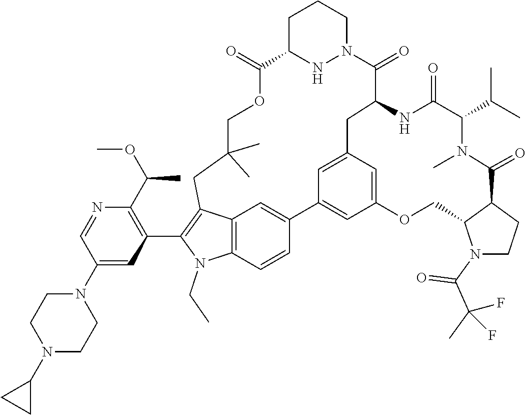 |
| A135 | 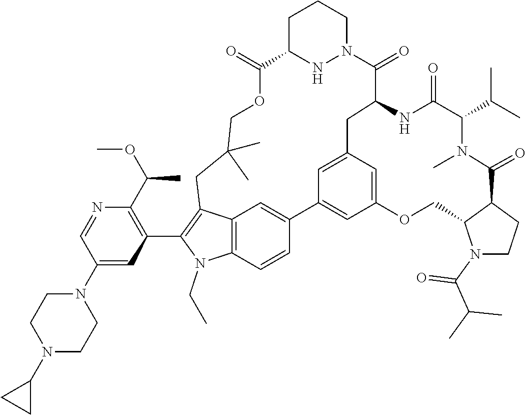 |
| A136 | 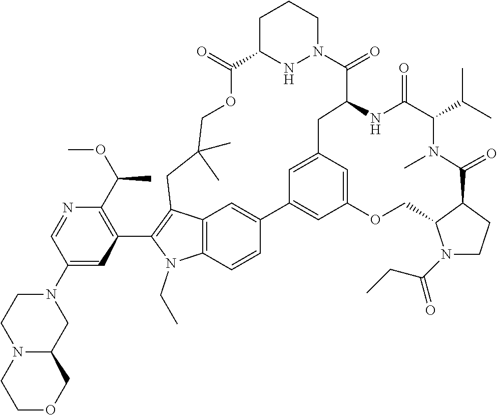 |
| A137 | 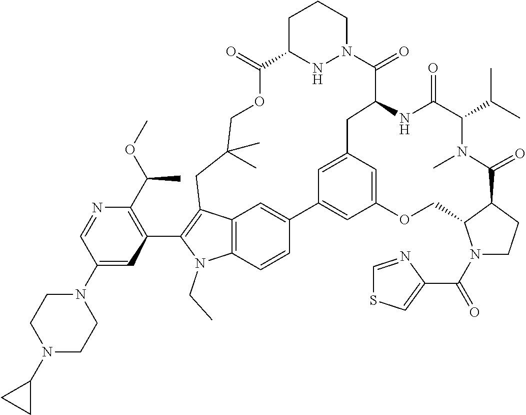 |
| A138 | 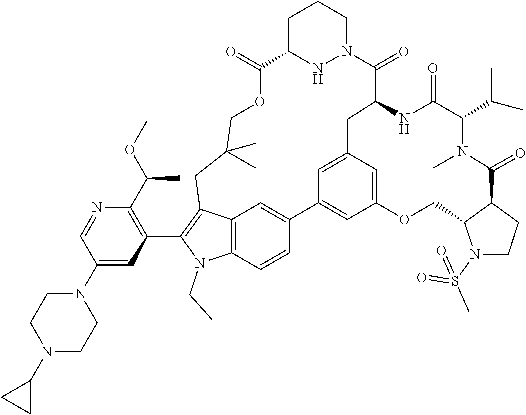 |
| A139 | 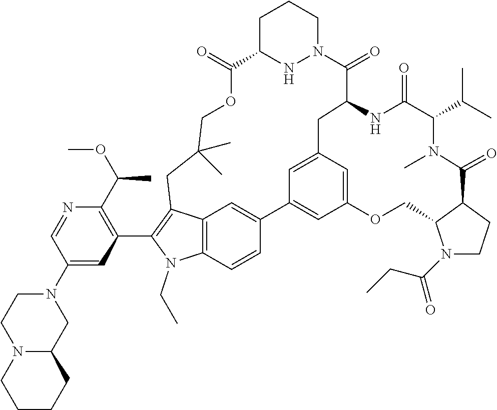 |
| A140 | 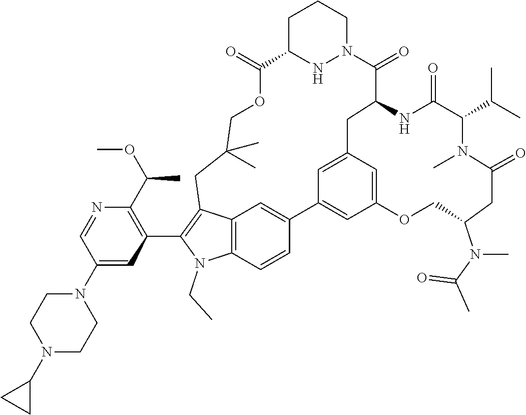 |
| A141 | 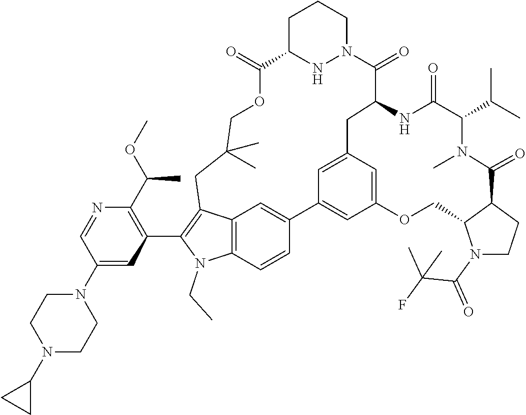 |
| A142 | 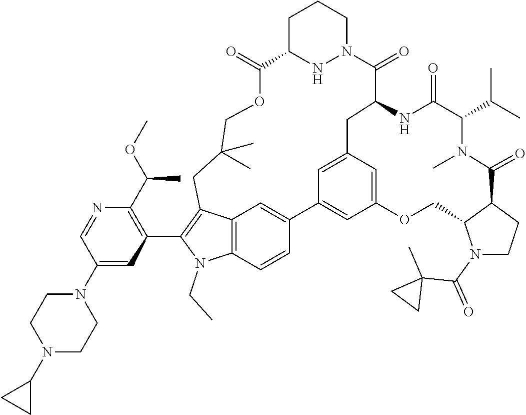 |
| A143 | 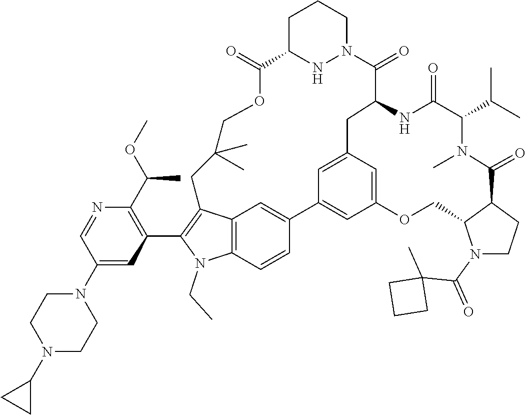 |
| A144 | 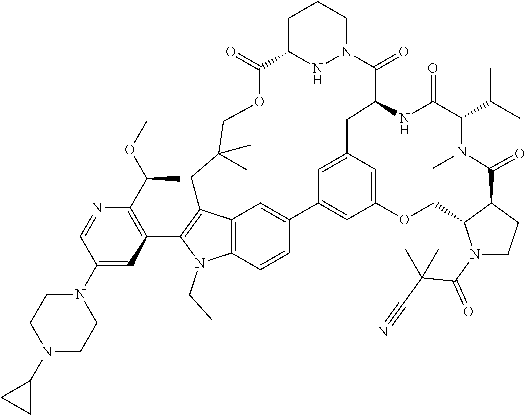 |
| A145 | 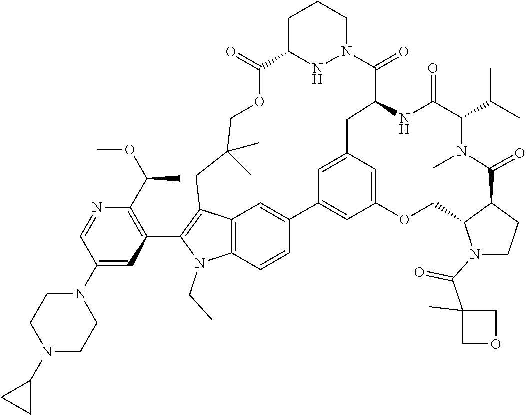 |
| A146 | 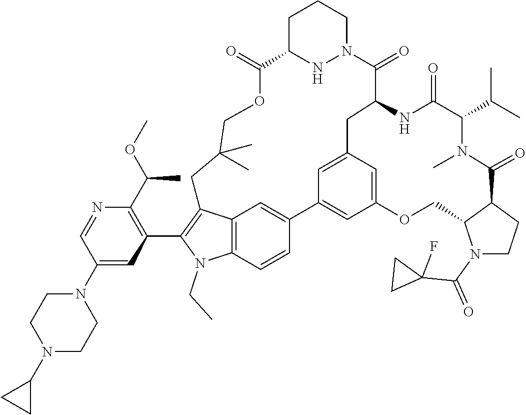 |
| A147 | 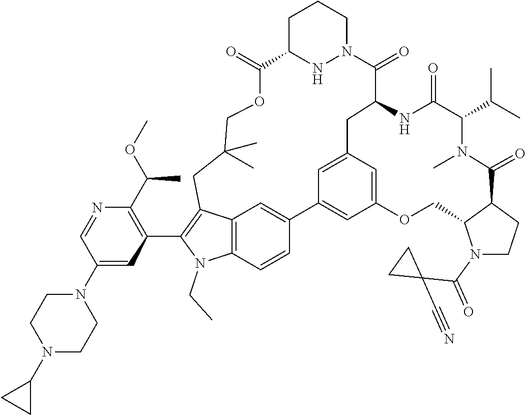 |
| A148 | 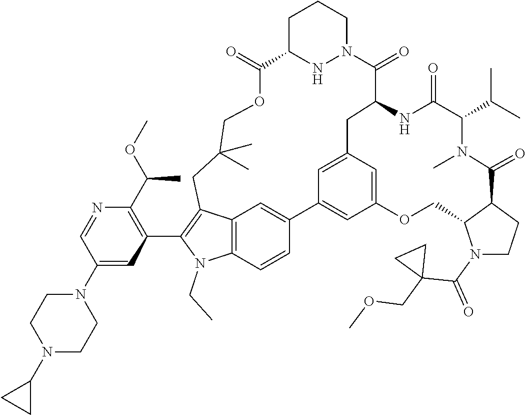 |
| A149 | 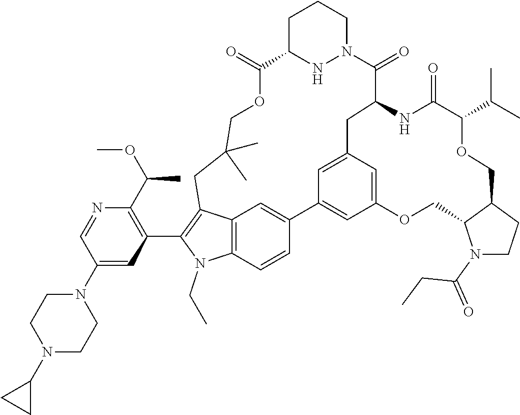 |
| A150 | 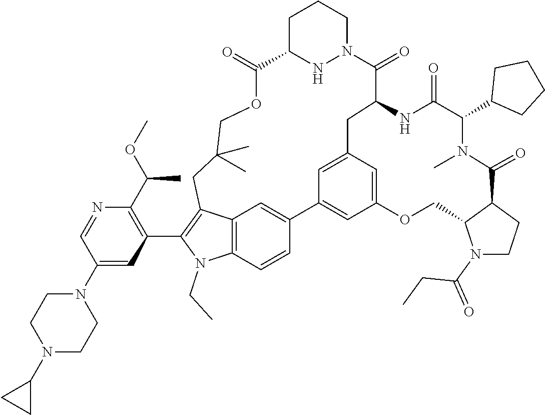 |
| A151 | 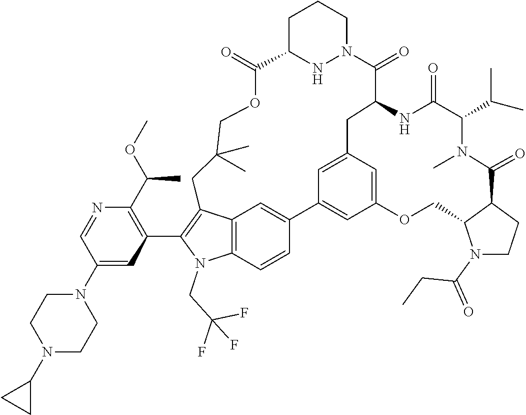 |
| A152 | 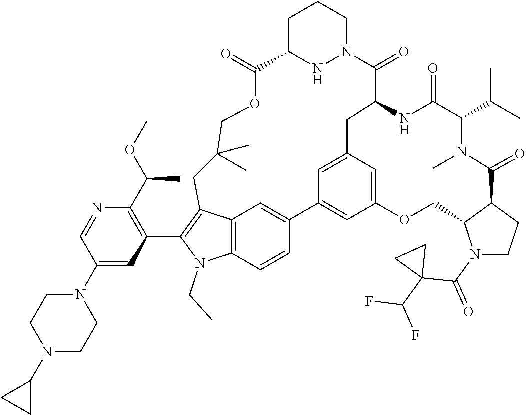 |
| A153 | 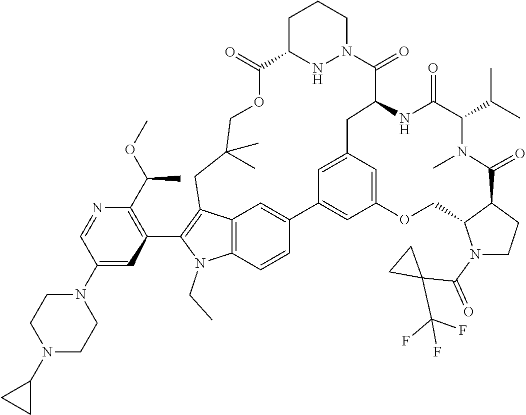 |
| A154 | 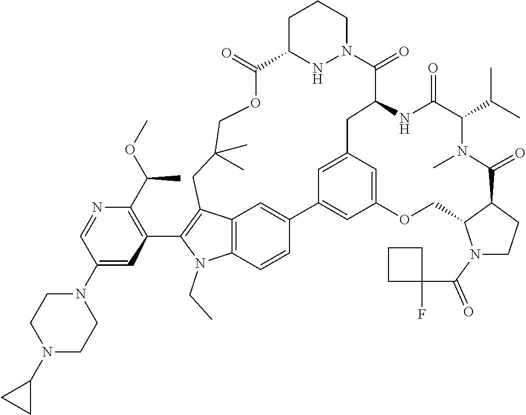 |
| A155 | 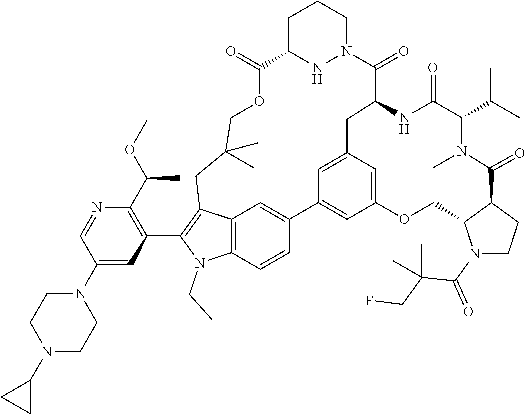 |
| A156 | 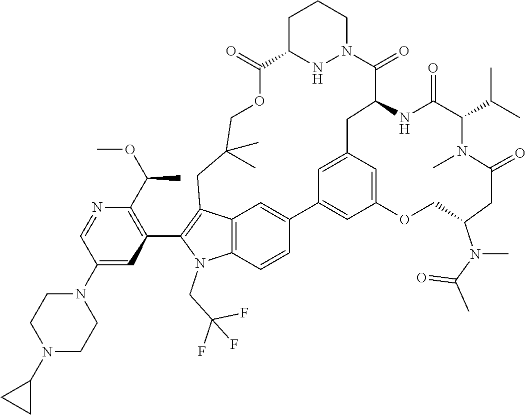 |
| A157 | 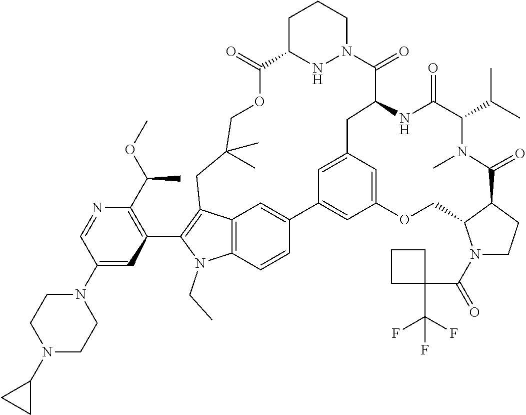 |
| A158 | 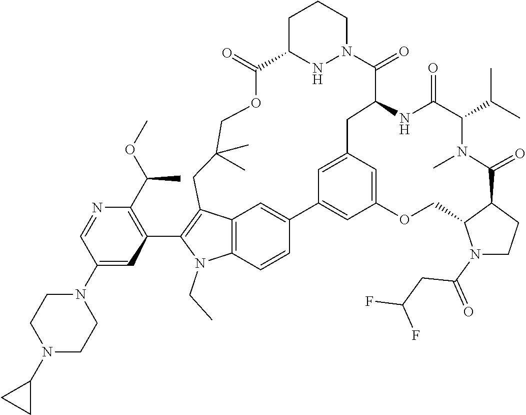 |
| A159 | 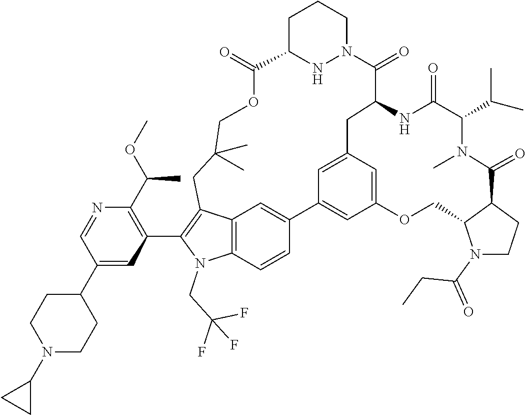 |
| A160 | 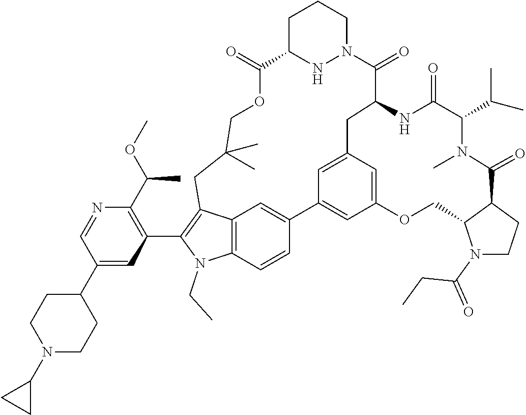 |
| A161 | 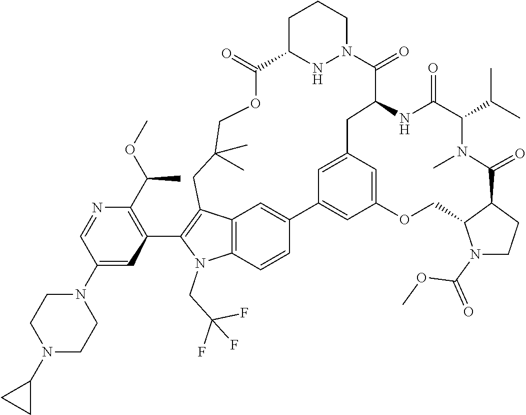 |
| A162 | 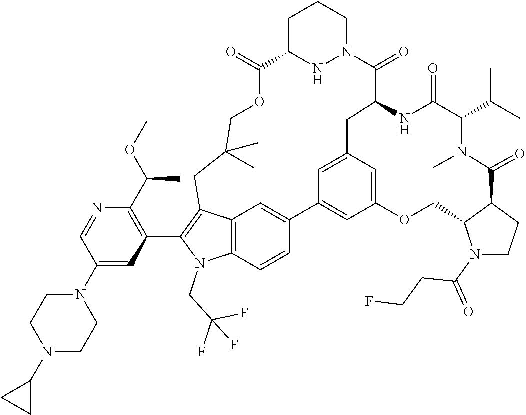 |
| A163 | 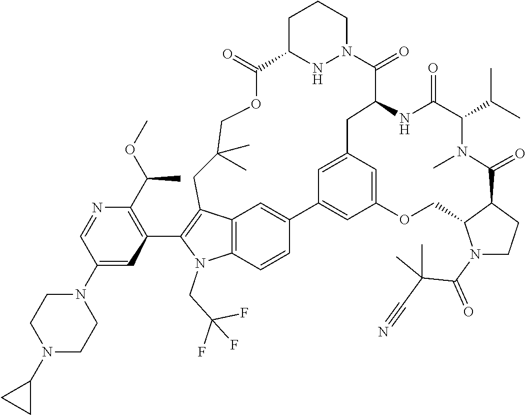 |
| A164 | 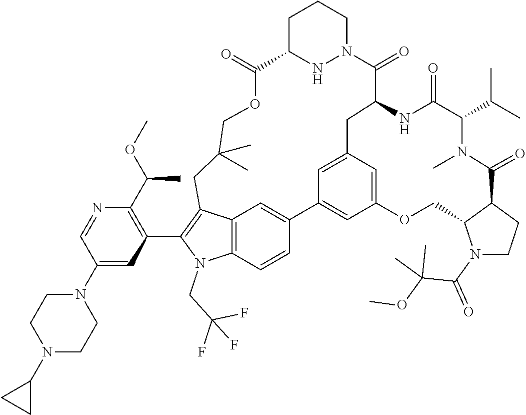 |
| A165 | 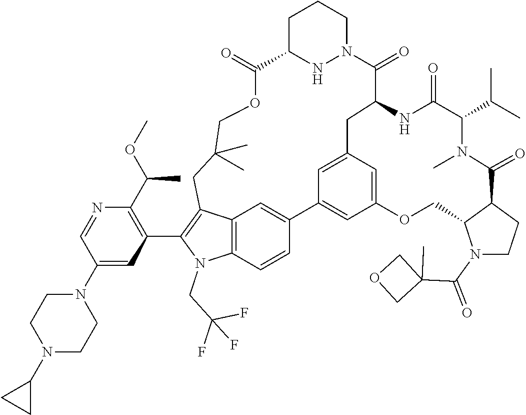 |
| A166 | 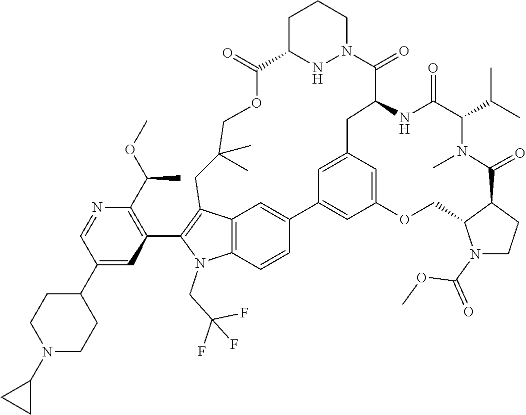 |
| A167 | 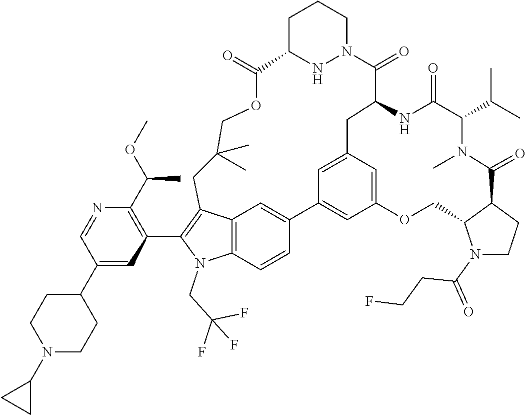 |
| A168 | 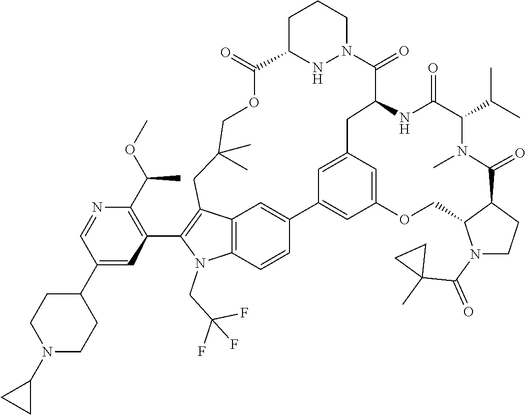 |
| A169 | 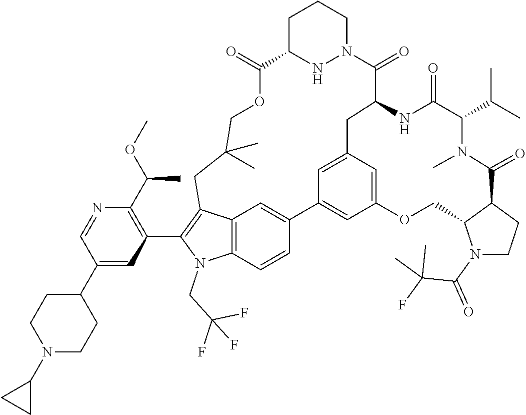 |
| A170 | 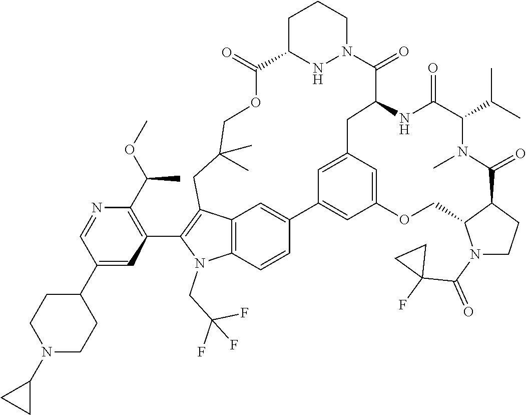 |
| A171 | 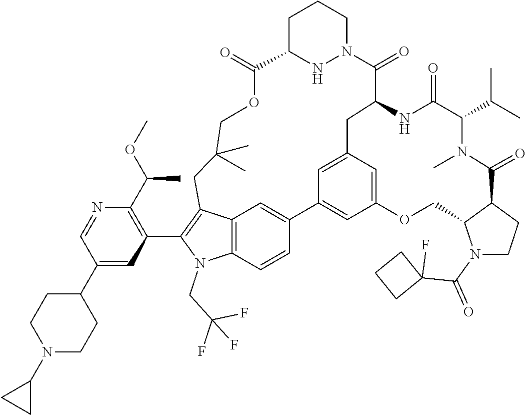 |
| A172 | 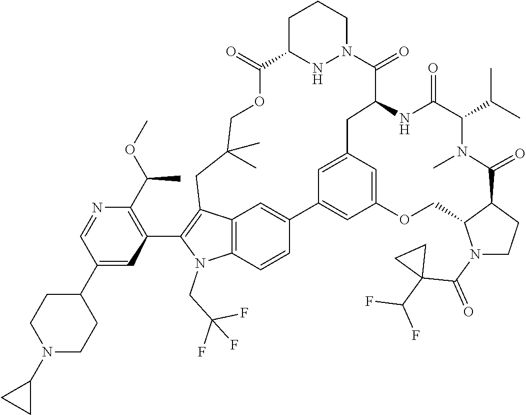 |
| A173 | 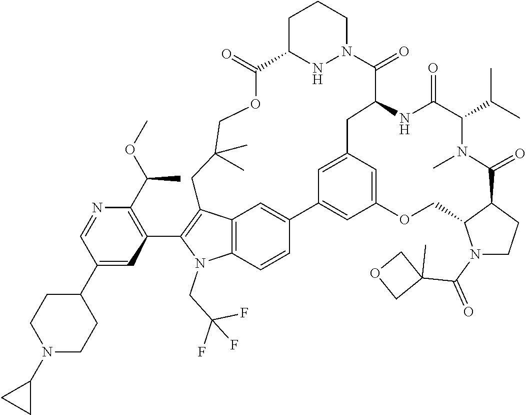 |
| A174 | 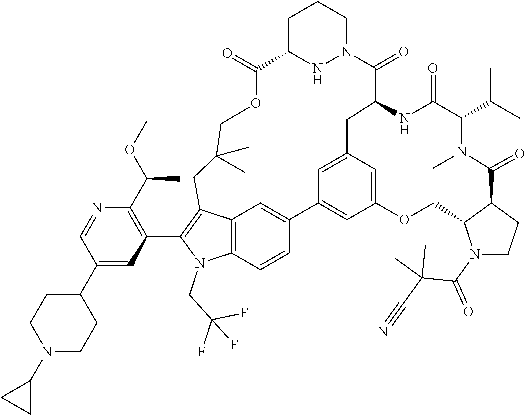 |
| A175 | 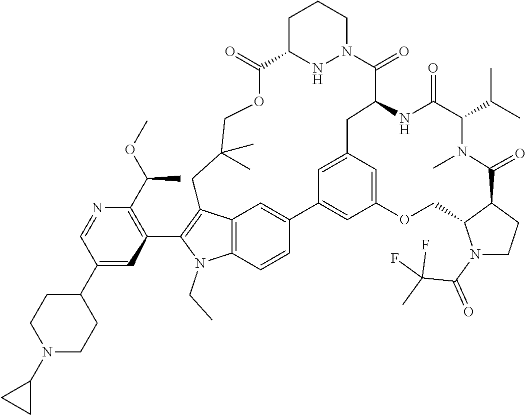 |
| A176 | 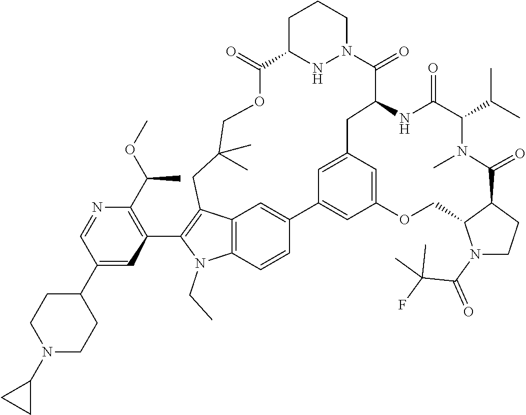 |
| A177 | 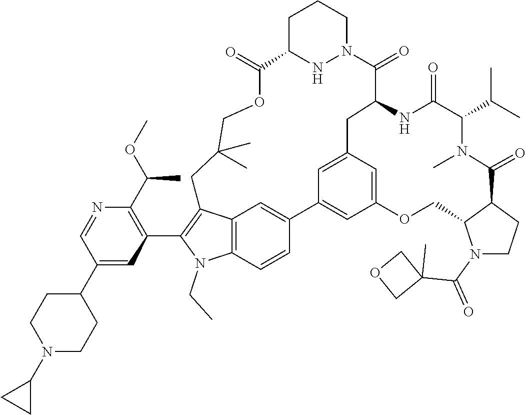 |
| A178 | 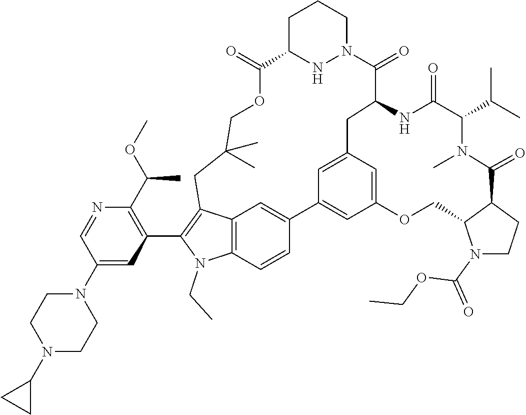 |
| A179 | 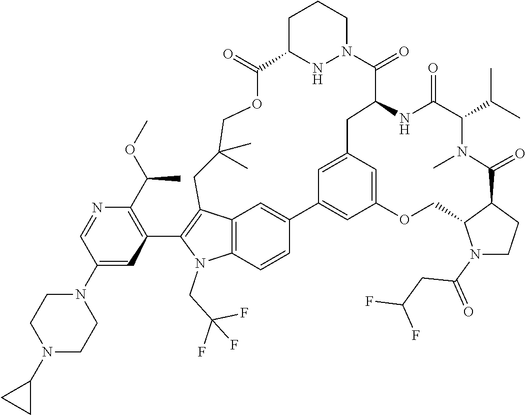 |
| A180 | 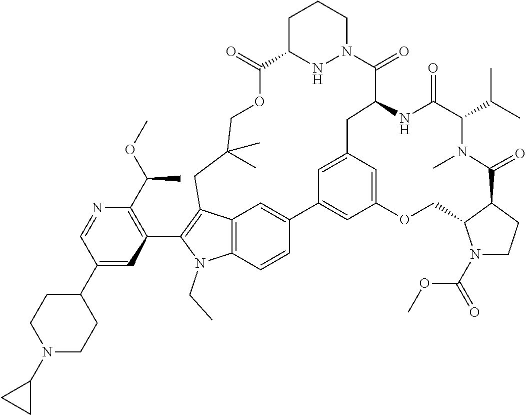 |
| A181 | 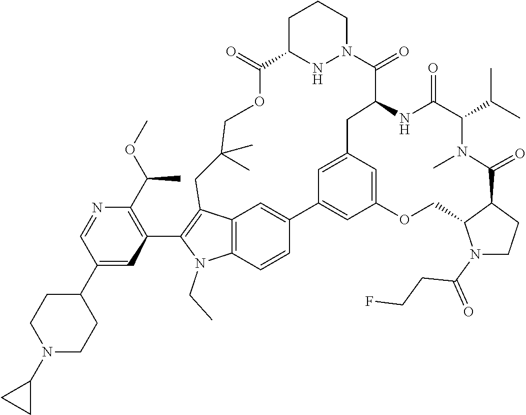 |
| A182 | 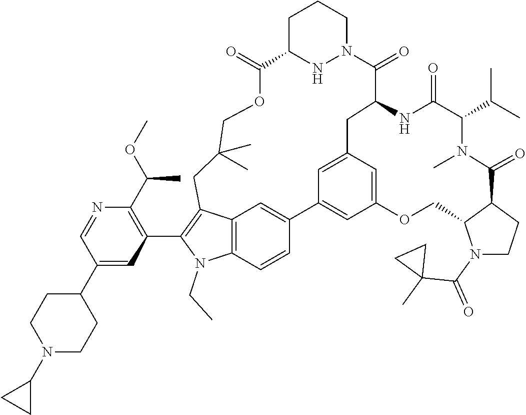 |
| A183 | 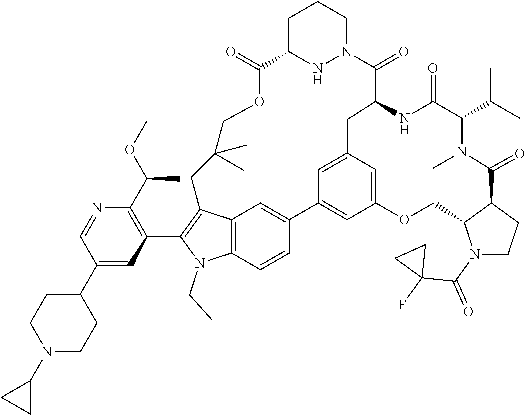 |
| A184 | 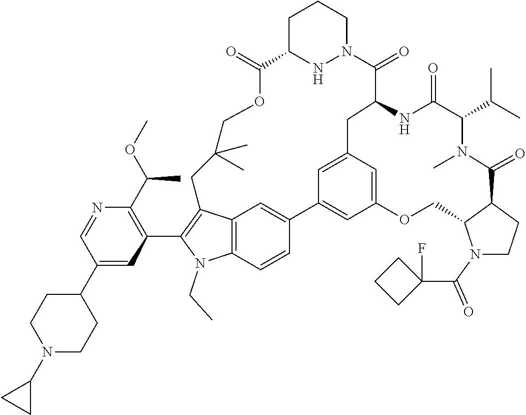 |
| A185 | 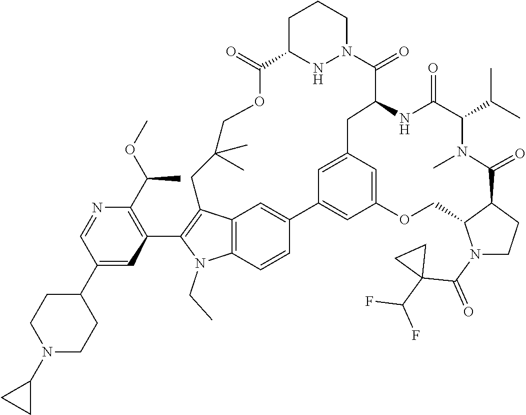 |
| A186 | 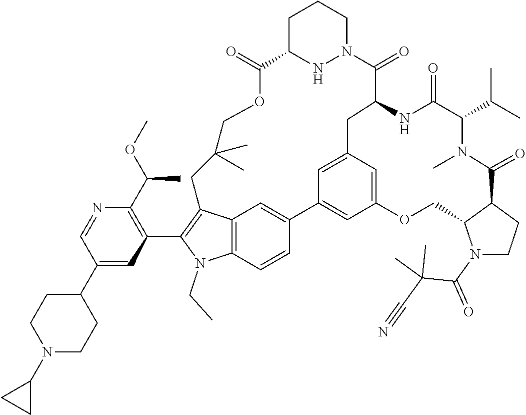 |
| A187 | 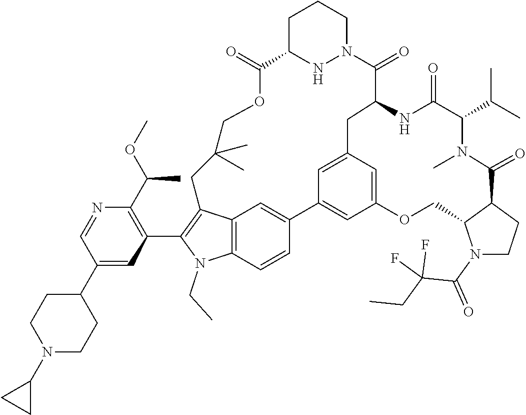 |
| A188 | 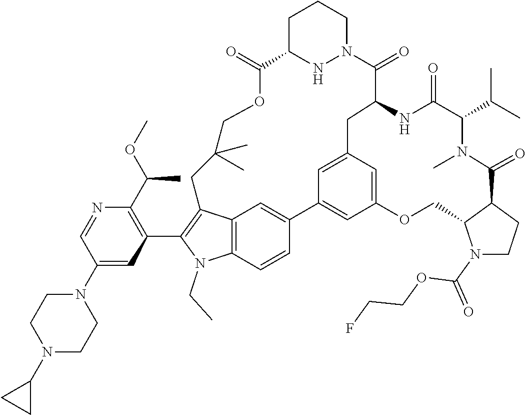 |
| A189 | 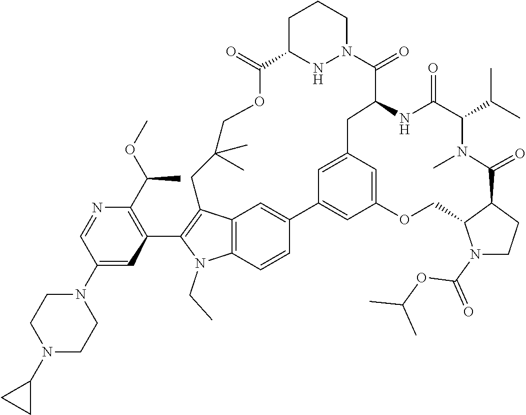 |
| A190 | 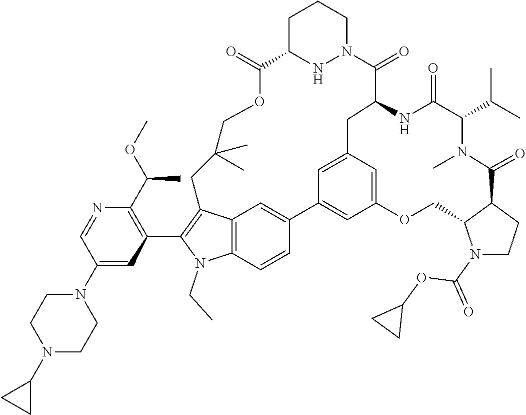 |
| A191 | 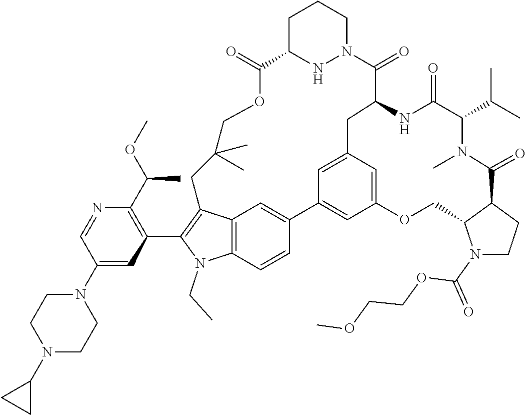 |
| A192 | 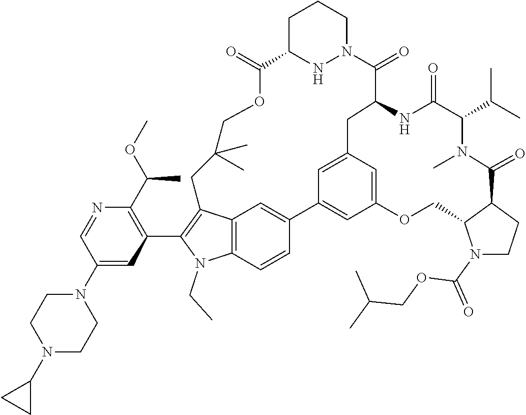 |
| A193 | 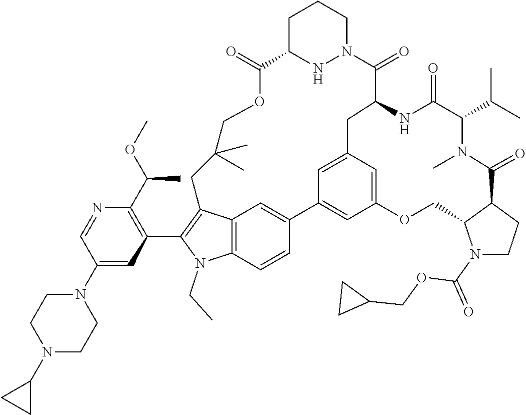 |
| A194 | 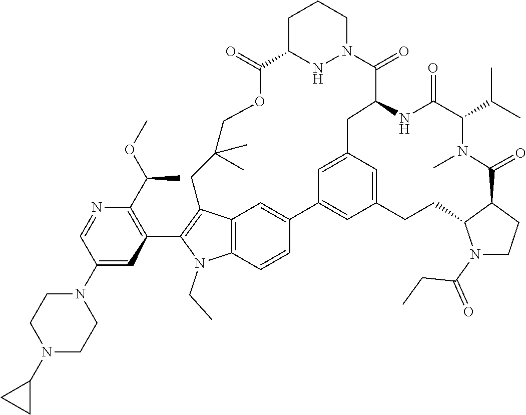 |
| A195 | 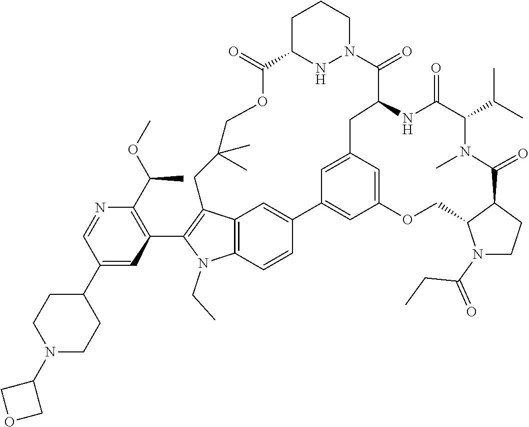 |
| A196 | 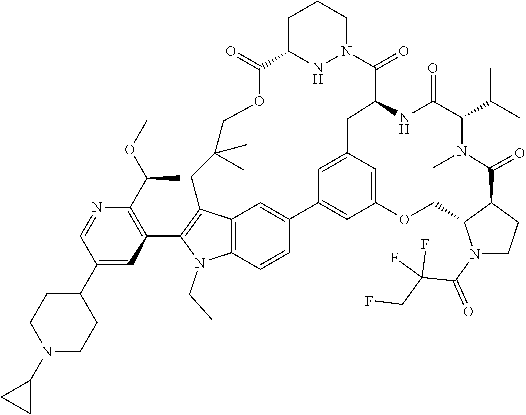 |
| A197 | 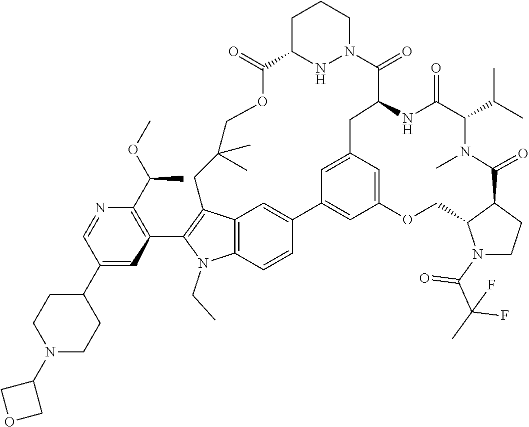 |
| A198 | 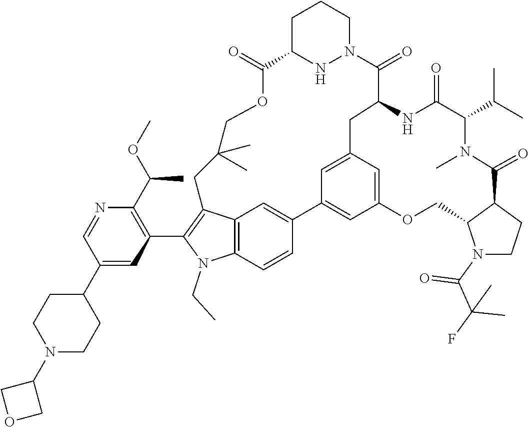 |
| A199 | 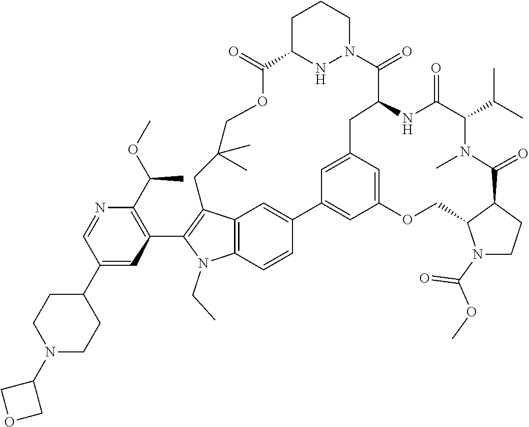 |
| A200 | 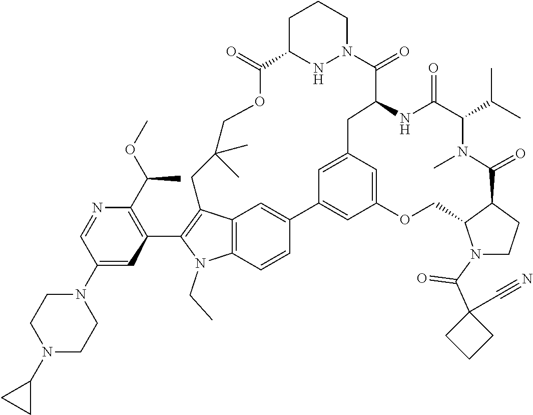 |
| A201 | 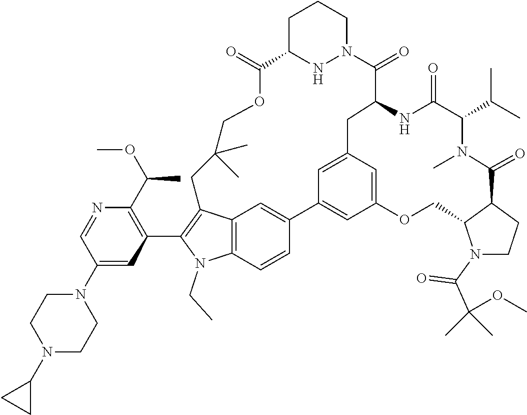 |
| A202 | 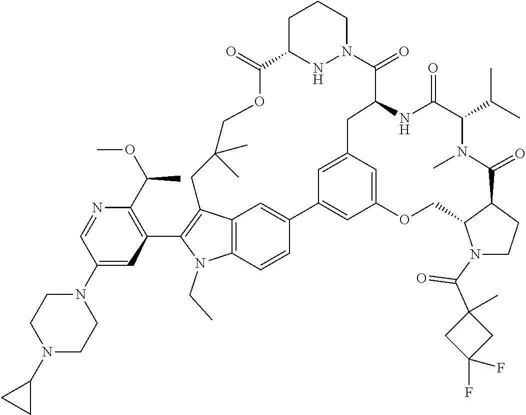 |
| A203 | 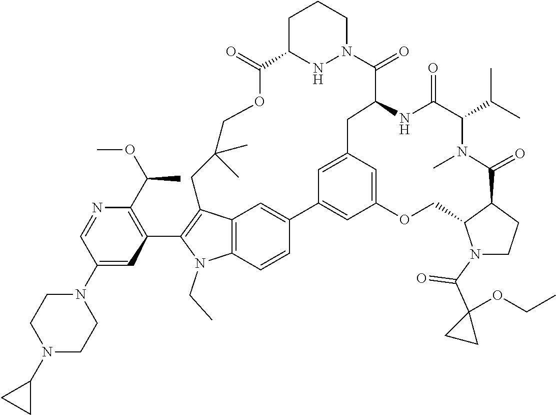 |
| A204 | 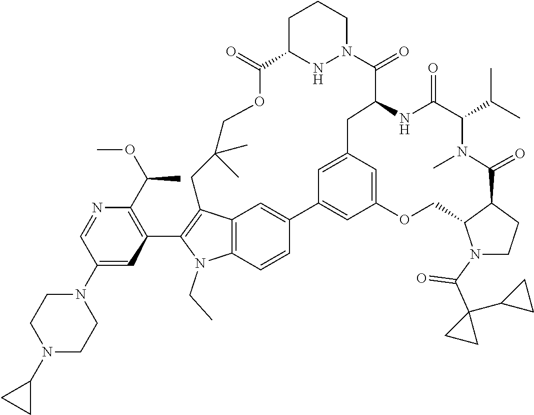 |
| A205 | 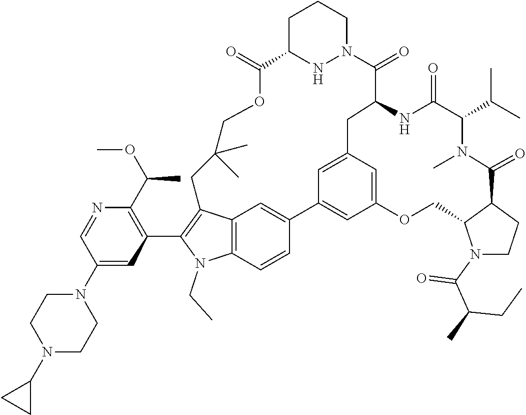 |
| A206 | 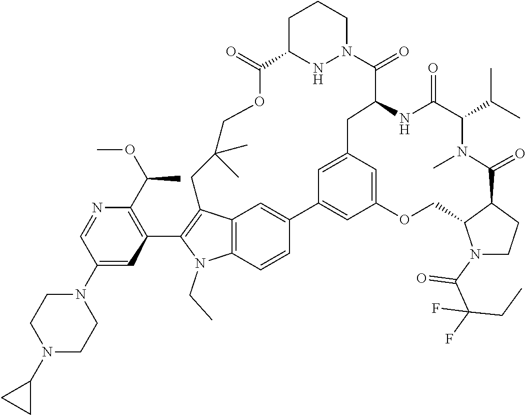 |
| A207 | 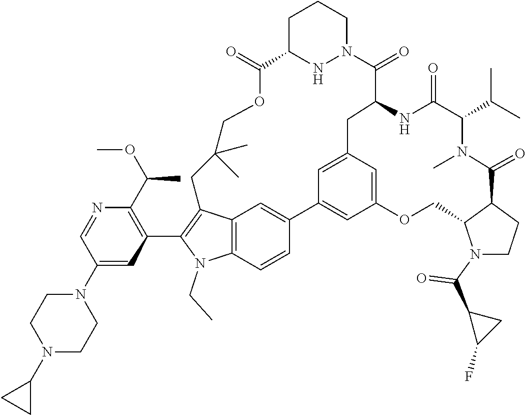 |
| A208 | 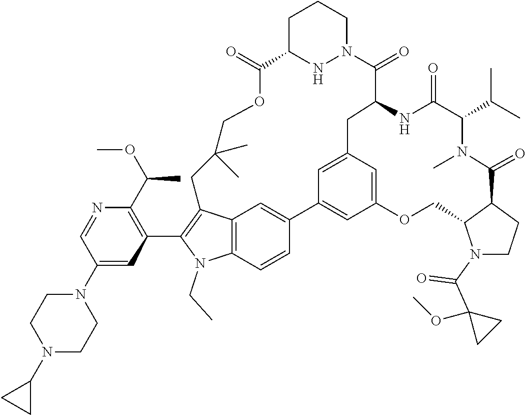 |
| A209 | 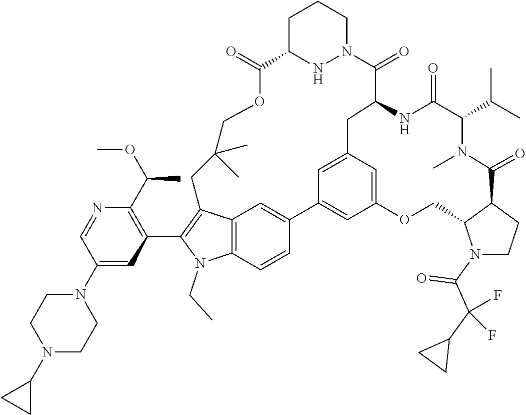 |
| A210 | 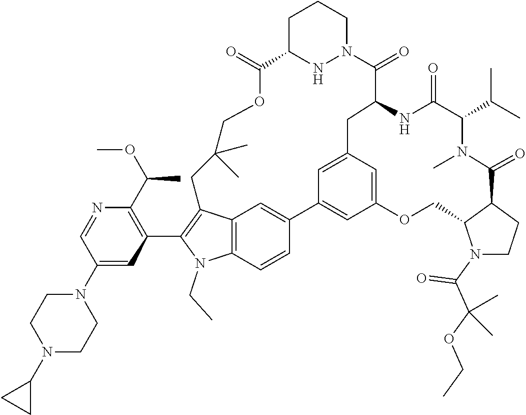 |
| A211 | 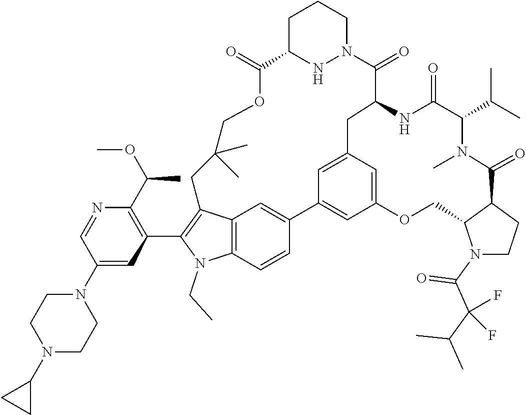 |
| A212 | 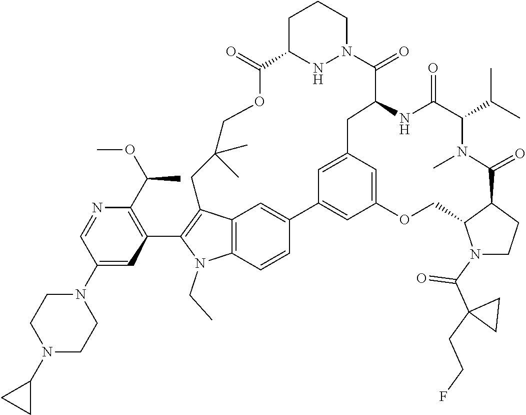 |
| A213 | 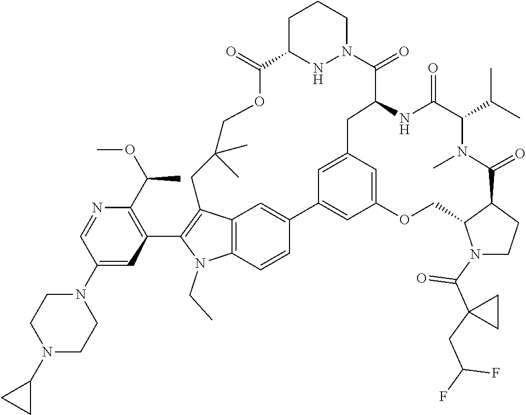 |
| A214 | 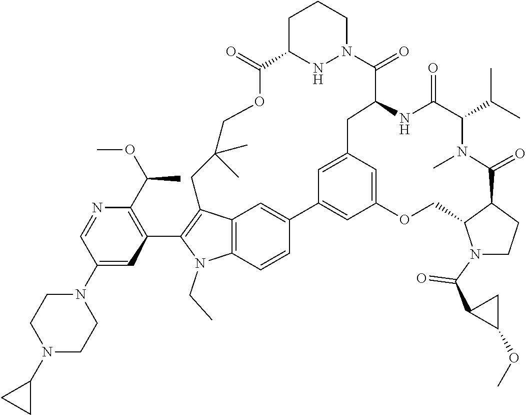 |
| A215 | 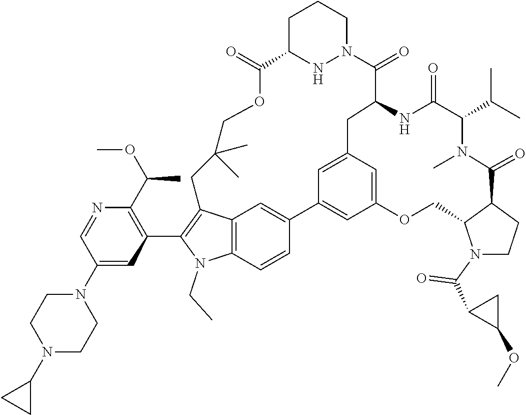 |
| A216 | 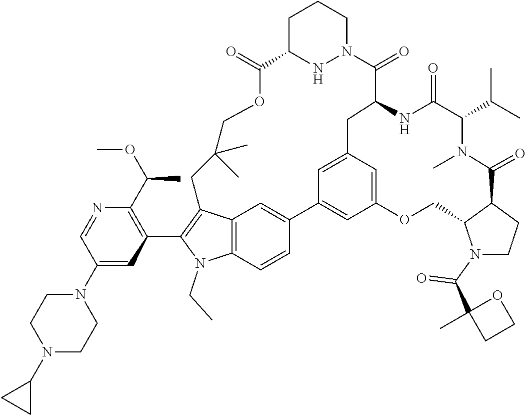 |
| A217 | 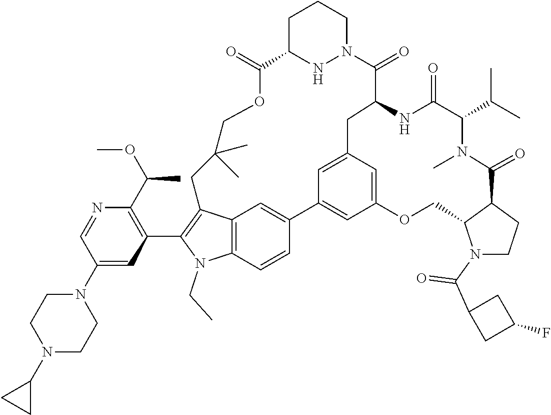 |
| A218 | 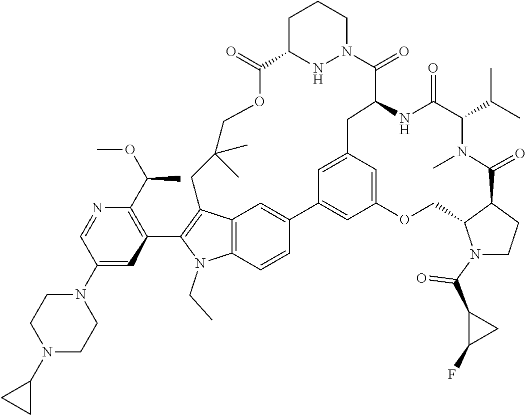 |
| A219 | 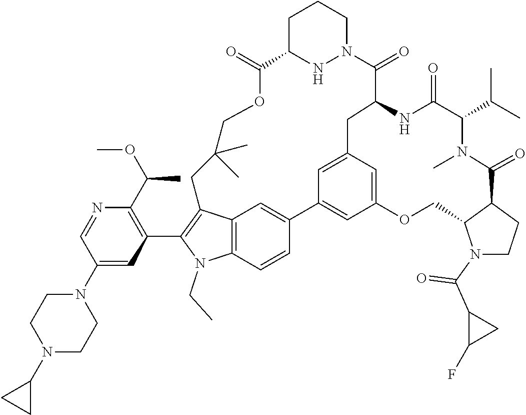 |
| A220 | 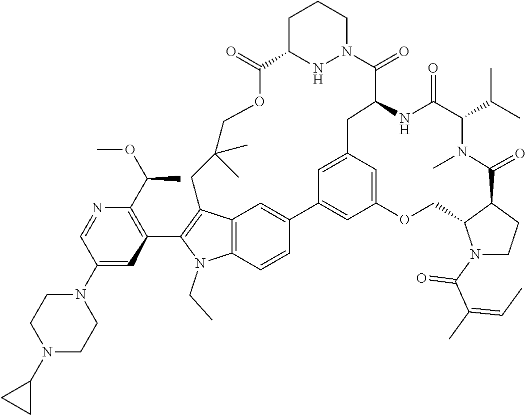 |
| A221 | 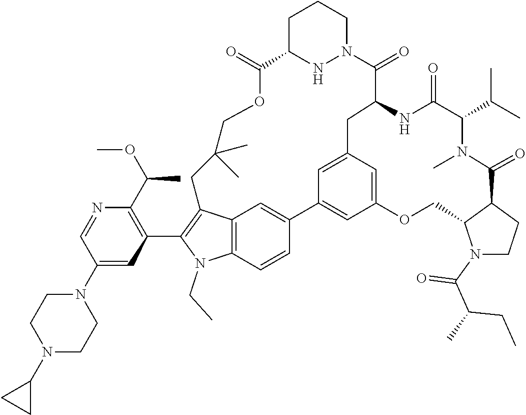 |
| A222 | 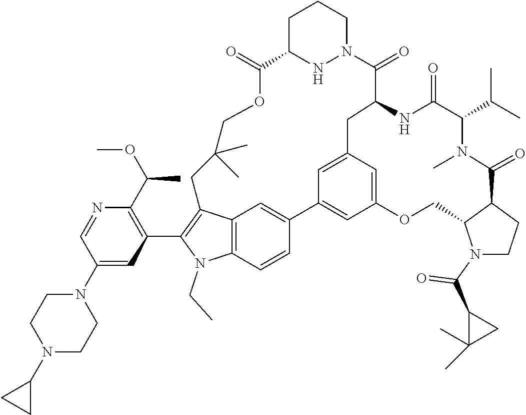 |
| A223 | 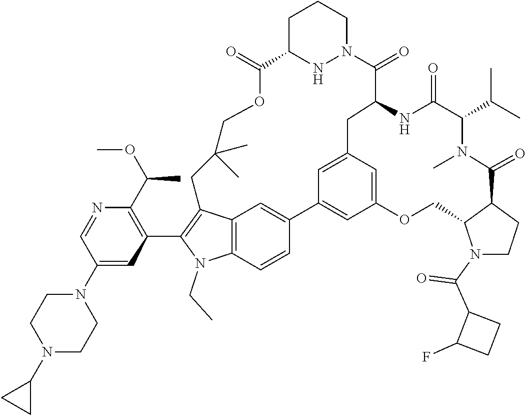 |
| A224 | 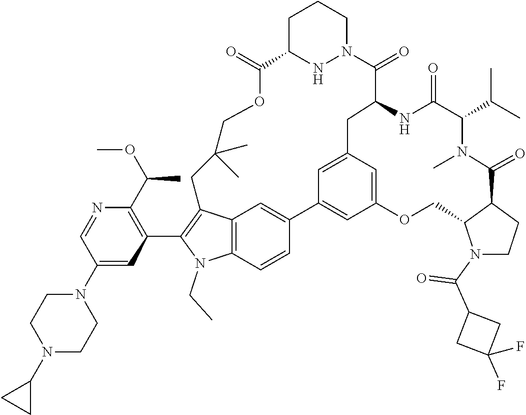 |
| A225 | 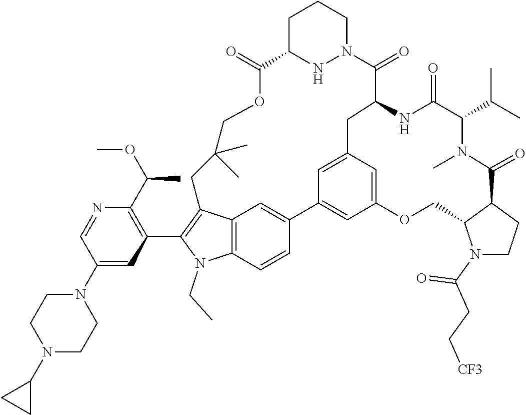 |
| A226 | 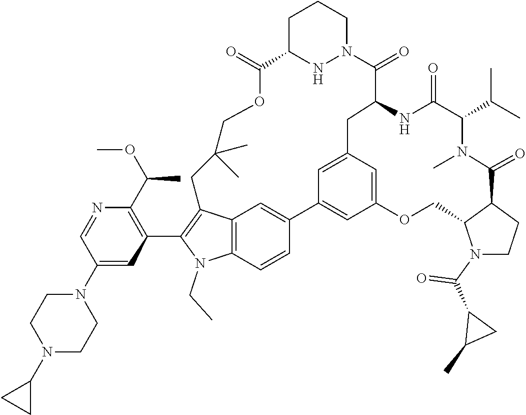 |
| A227 | 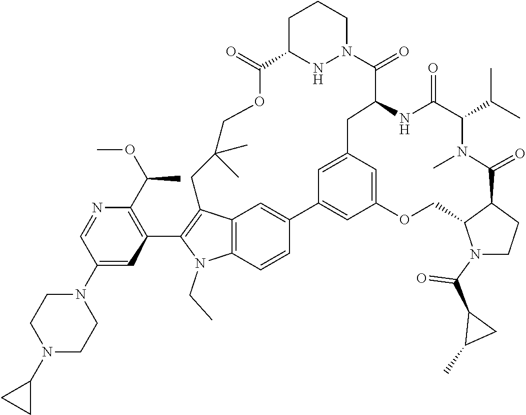 |
| A228 | 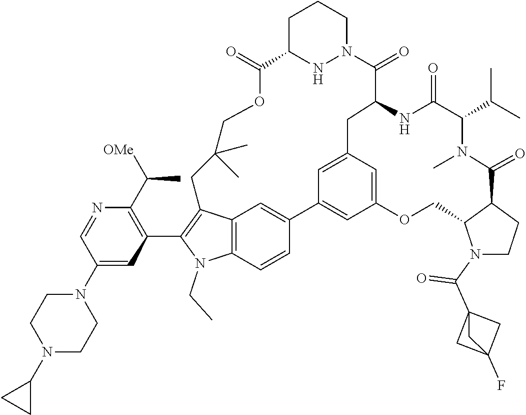 |
| A229 | 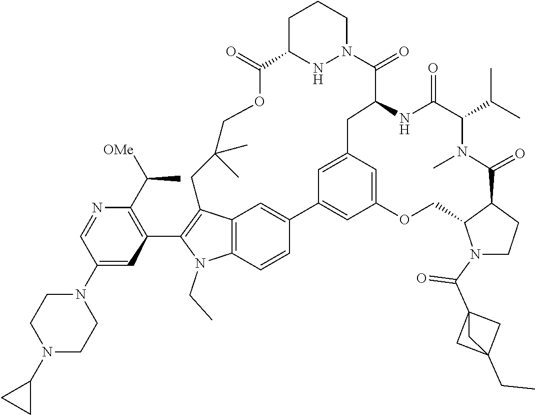 |
| A230 | 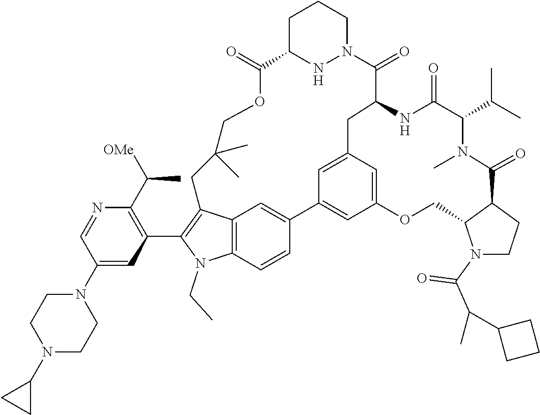 |
| A231 | 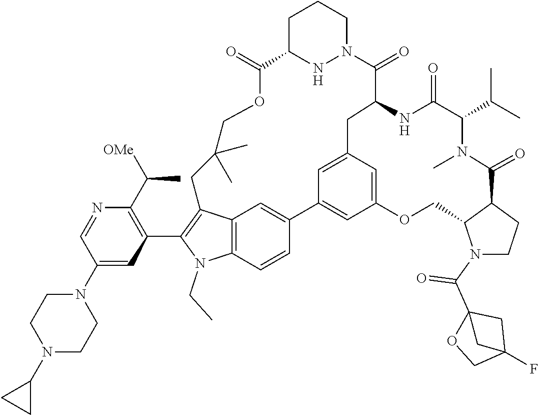 |
| A232 | 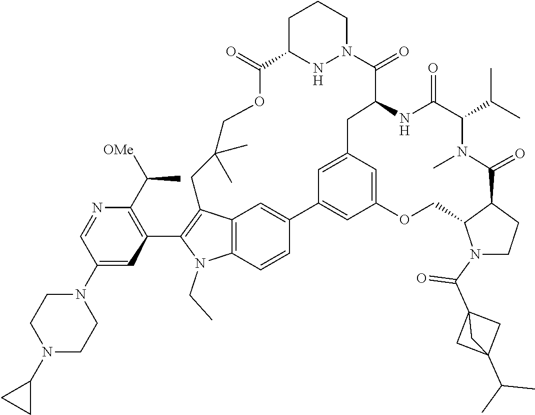 |
| A233 | 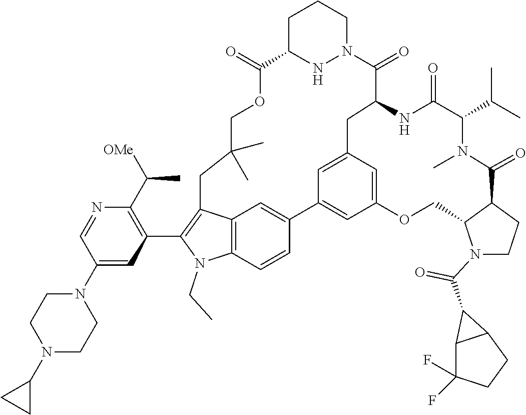 |
| A234 | 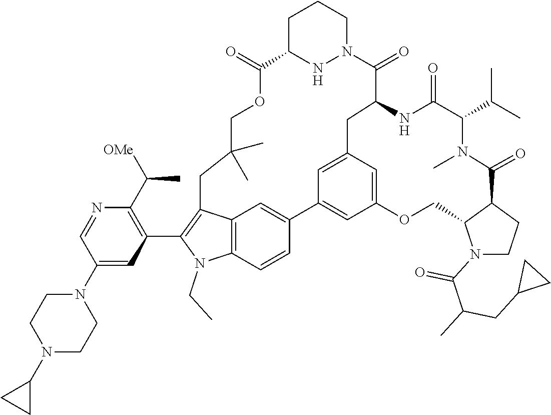 |
| A235 | 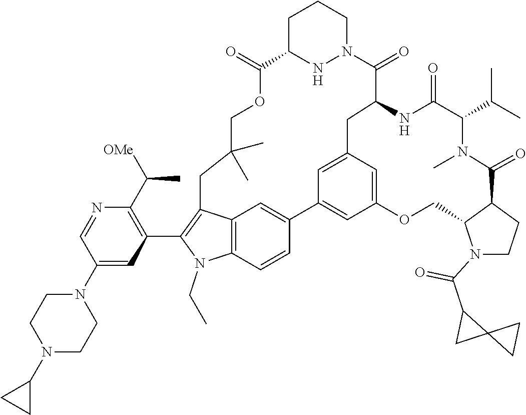 |
| A236 | 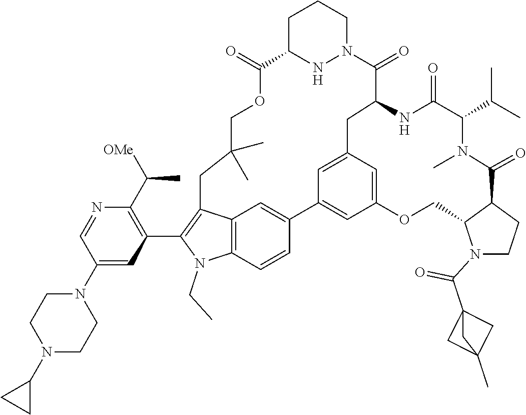 |
| A237 | 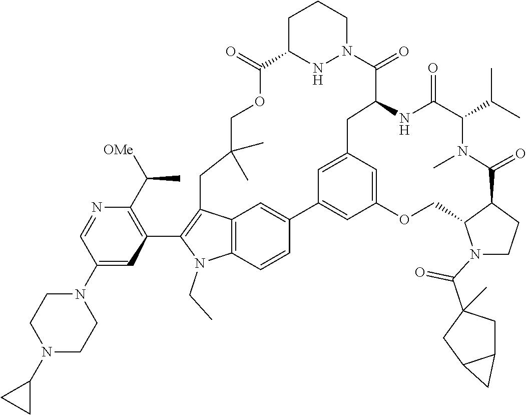 |
| A238 | 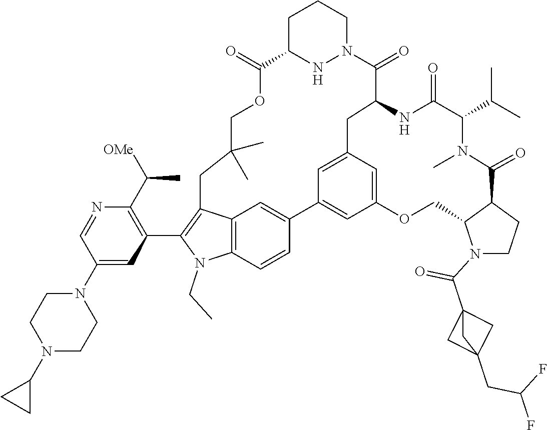 |
| A239 | 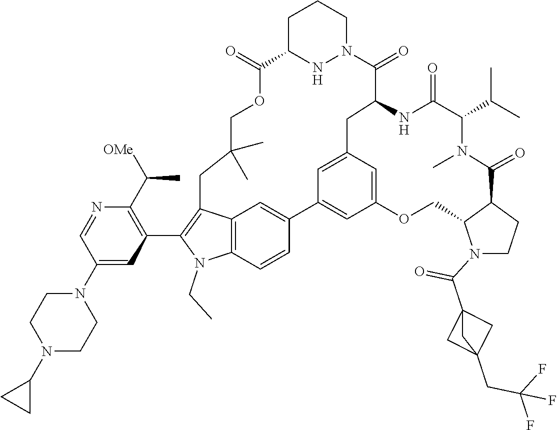 |
| A240 | 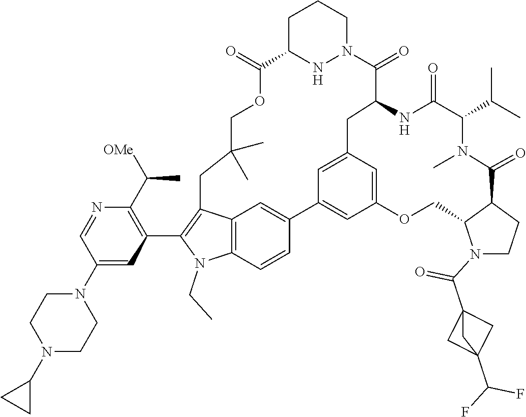 |
| A241 | 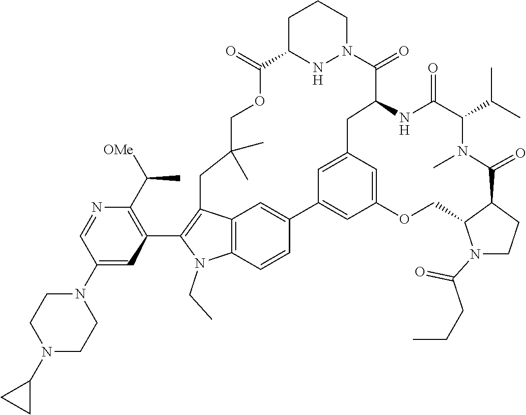 |
| A242 | 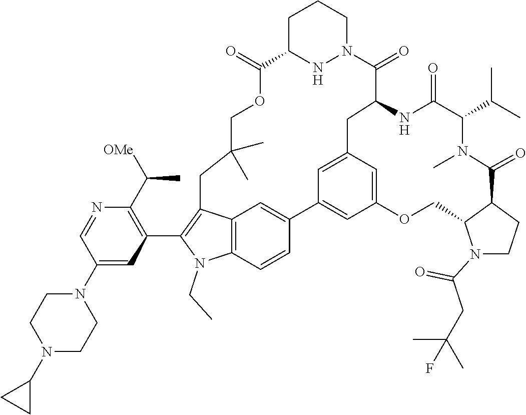 |
| A243 | 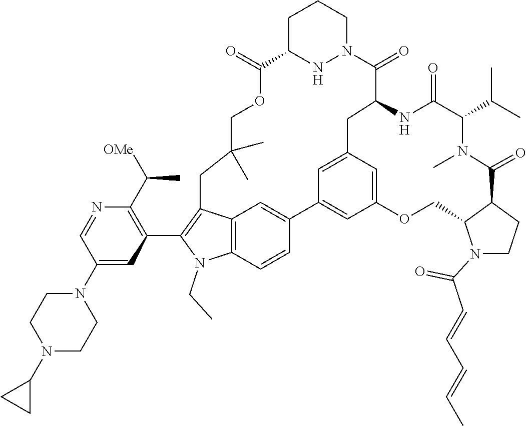 |
| A244 | 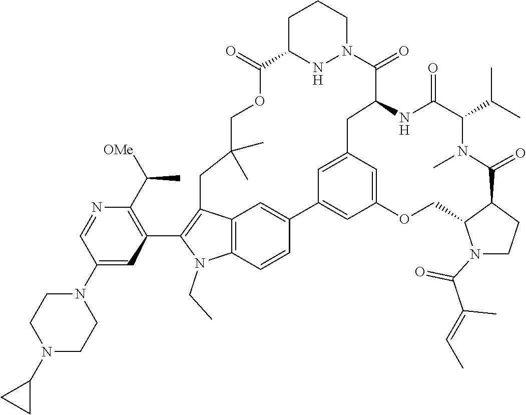 |
| A245 | 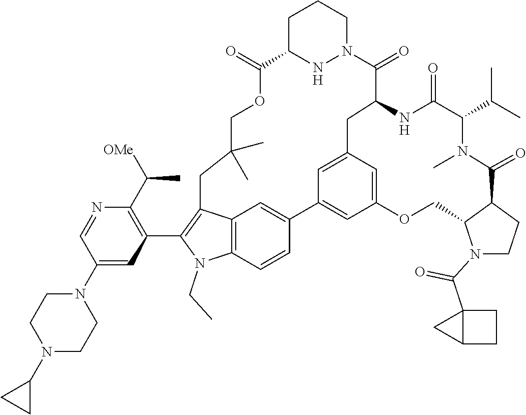 |
| A246 | 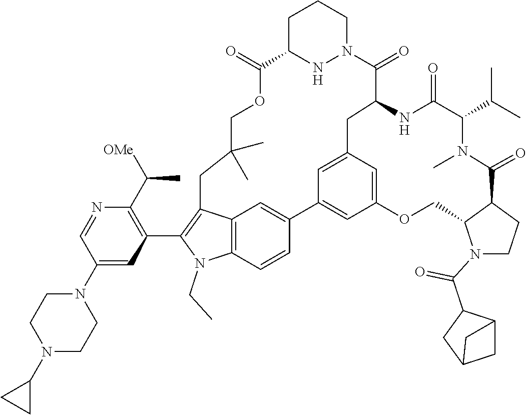 |
| A247 | 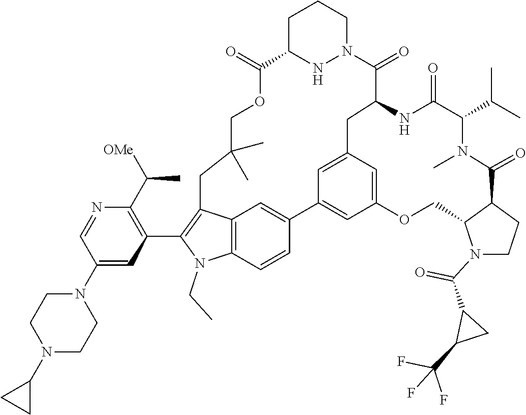 |
| A248 | 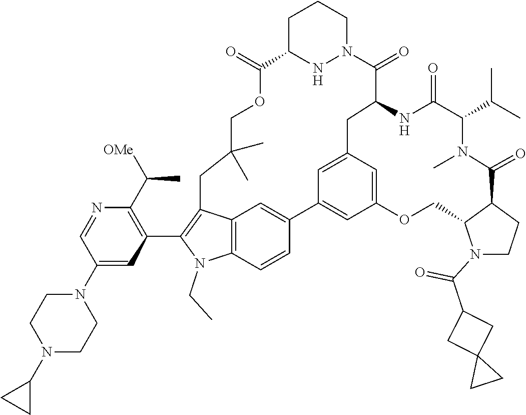 |
| A249 | 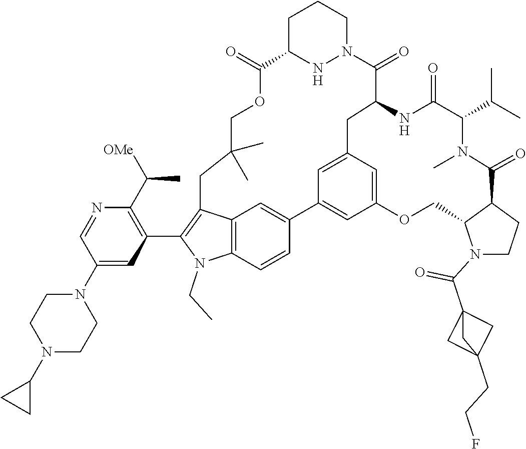 |
| A250 | 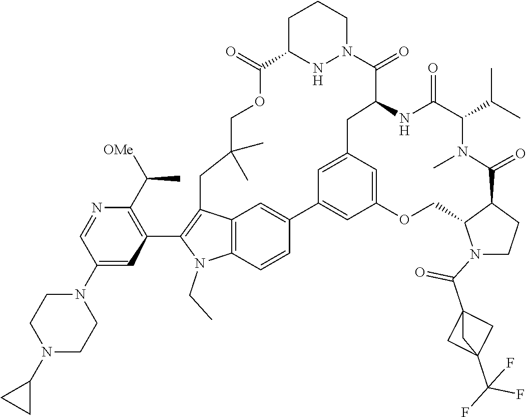 |
| A251 | 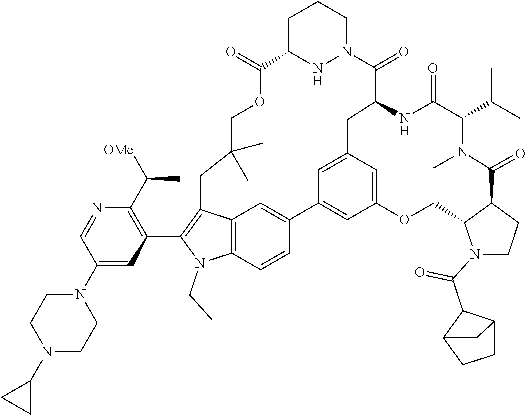 |
| A252 | 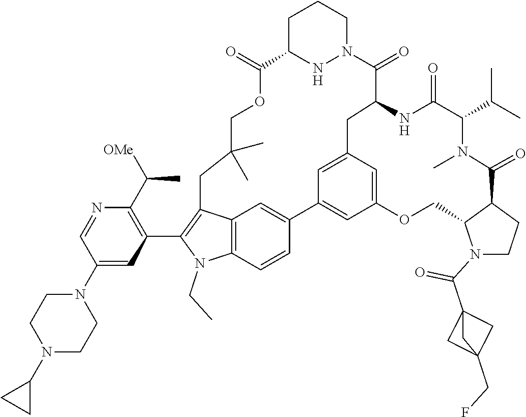 |
| A253 | 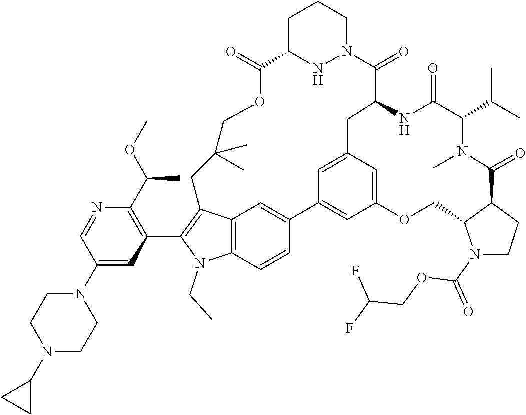 |
| A254 | 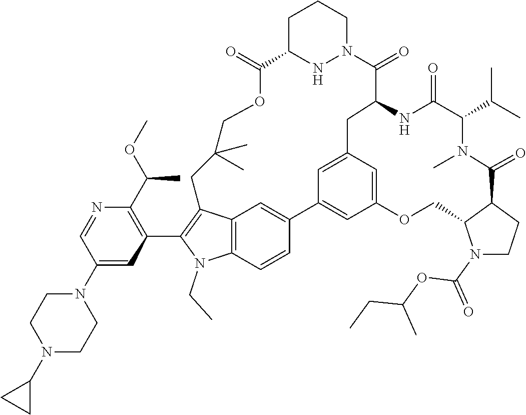 |
| A255 | 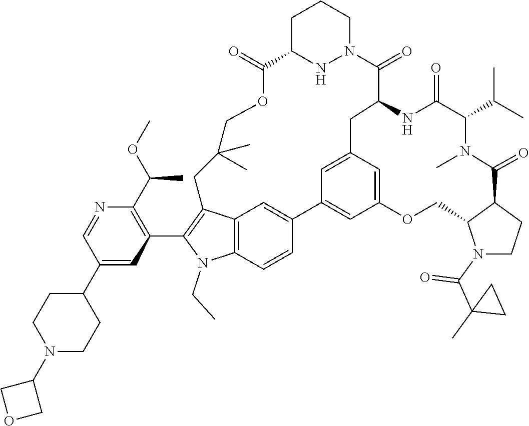 |
| A256 | 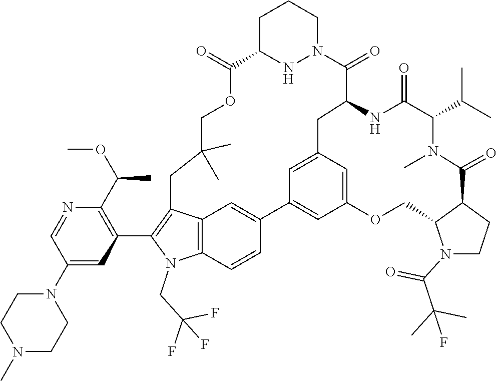 |
| A257 | 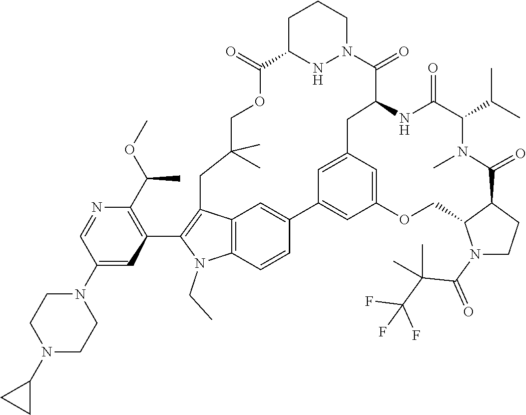 |
| A258 | 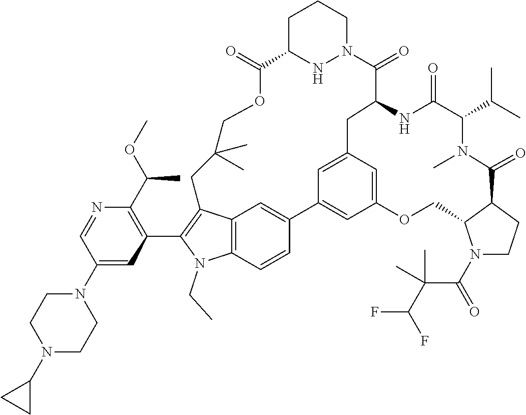 |
| A259 | 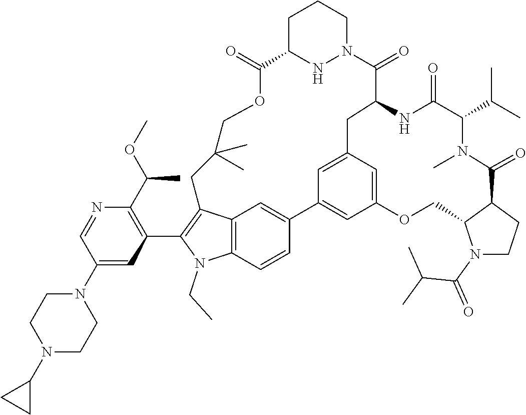 |
| A260 | 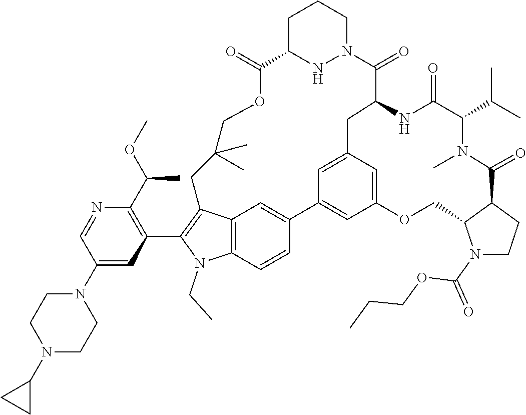 |
| A261 | 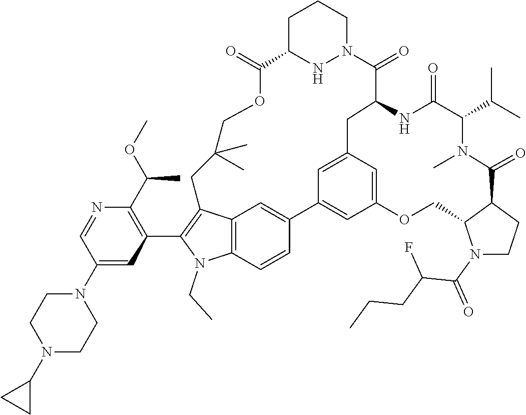 |
| A262 | 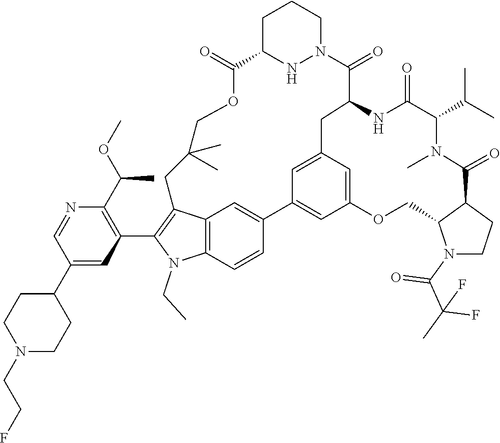 |
| A263 | 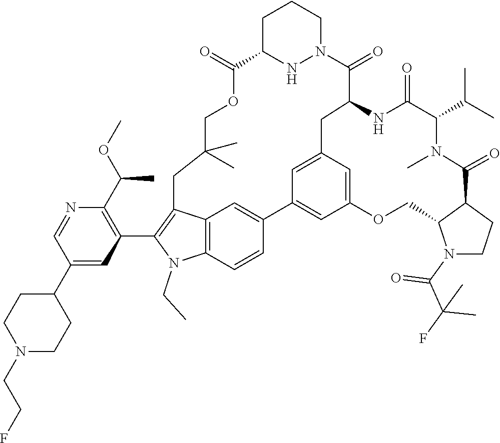 |
| A264 | 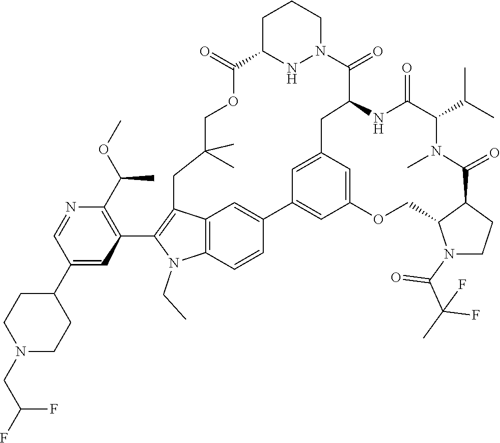 |
| A265 | 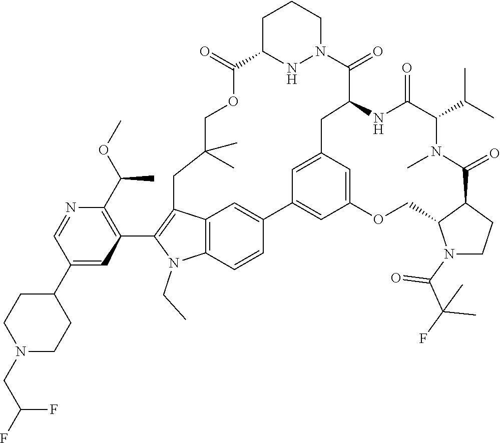 |
| A266 | 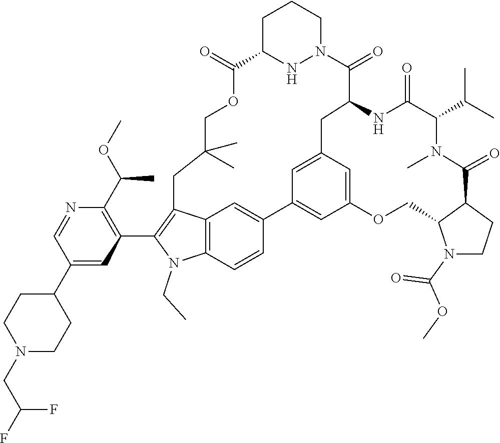 |
| A267 | 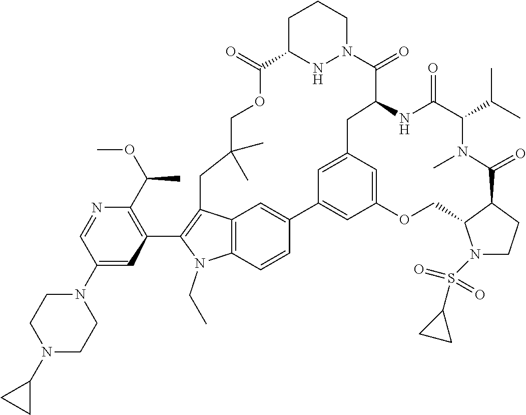 |
| A268 | 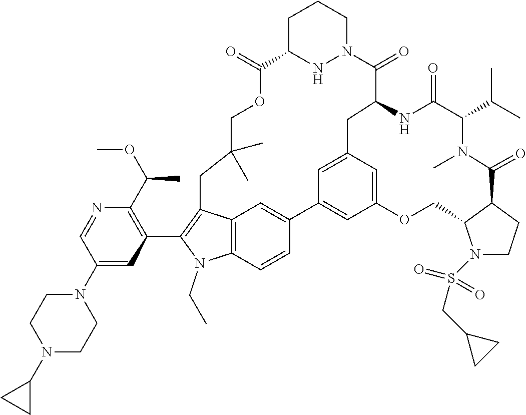 |
| A269 | 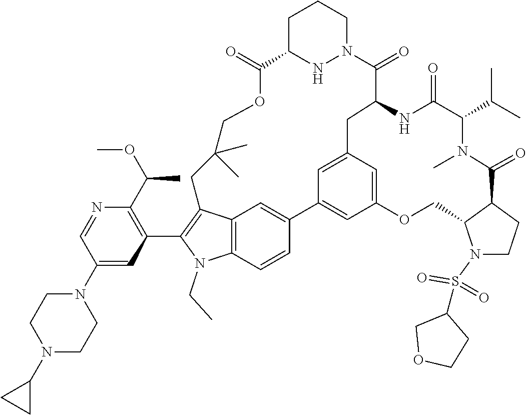 |
| A270 | 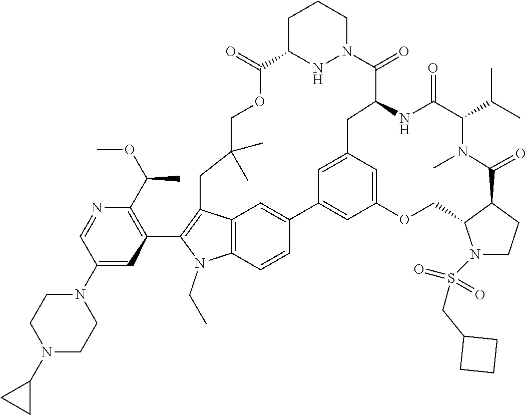 |
| A271 | 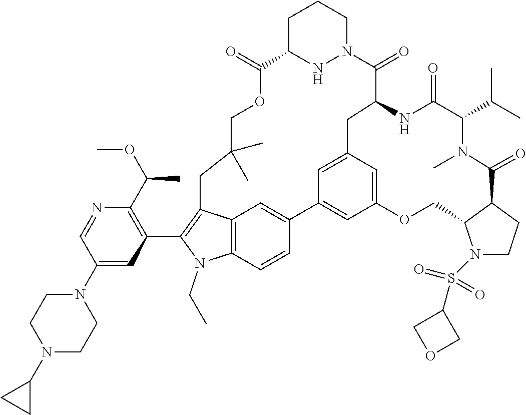 |
| A272 | 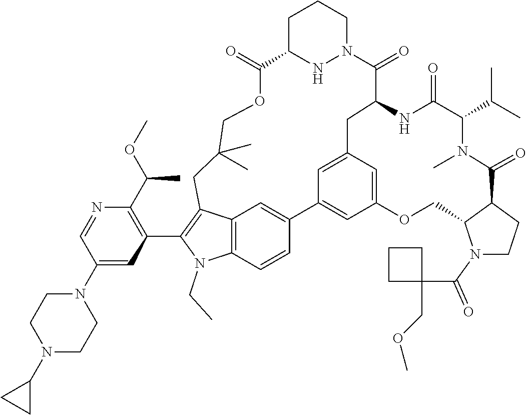 |
| A273 | 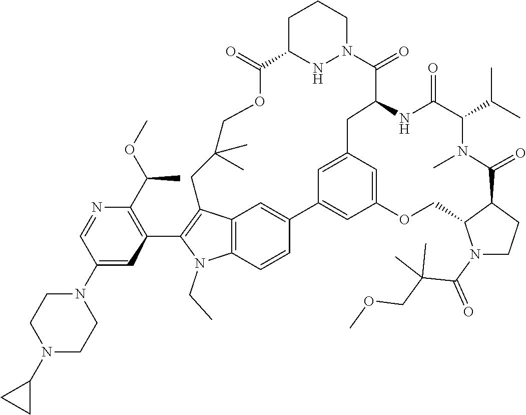 |
| A274 | 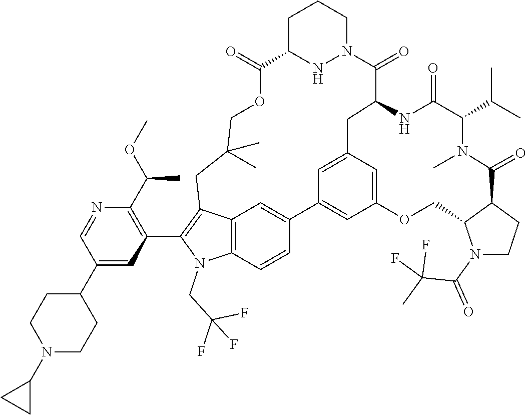 |
| A275 | 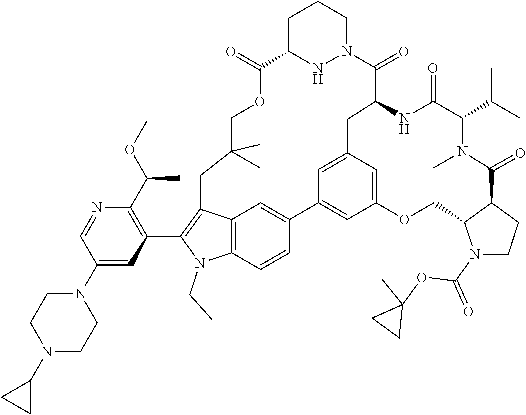 |
| A276 | 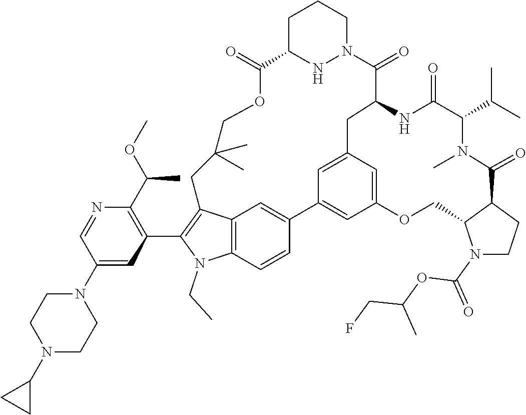 |
| A277 | 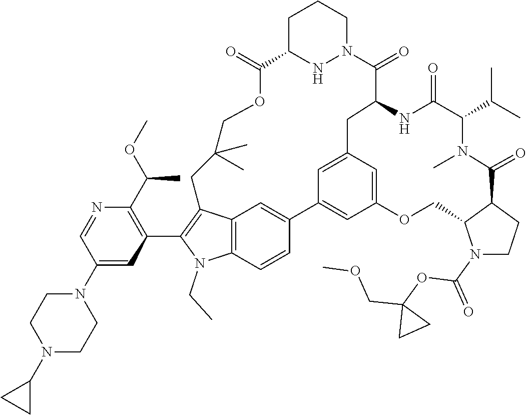 |
| A278 | 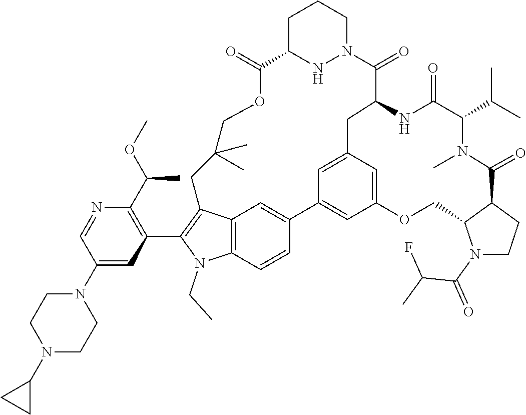 |
| A279 | 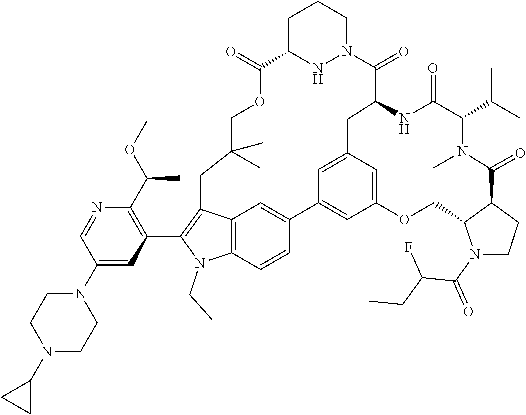 |
| A280 | 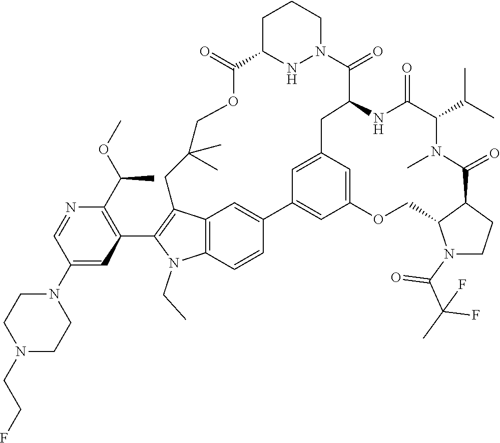 |
| A281 | 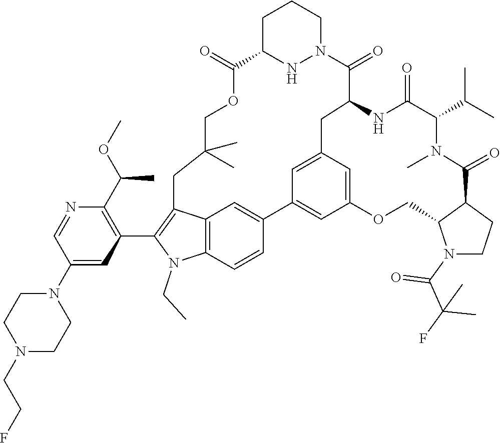 |
| A282 | 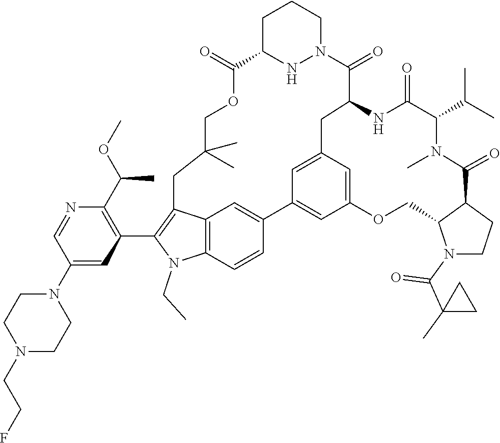 |
| A283 | 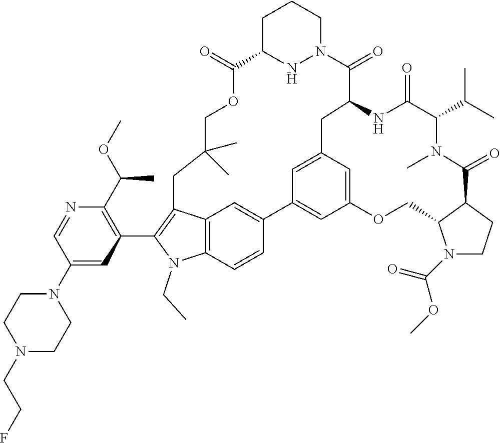 |
| A284 | 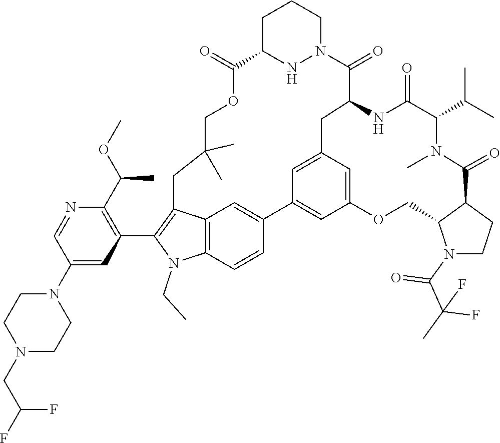 |
| A285 | 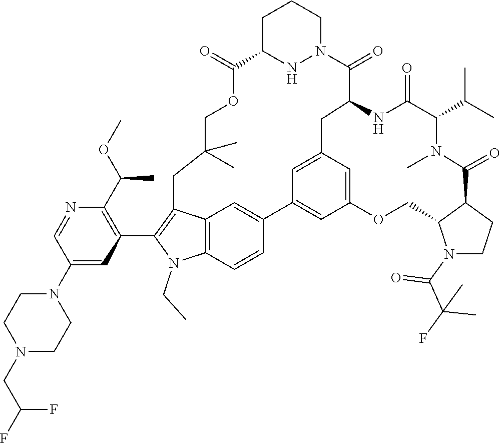 |
| A286 | 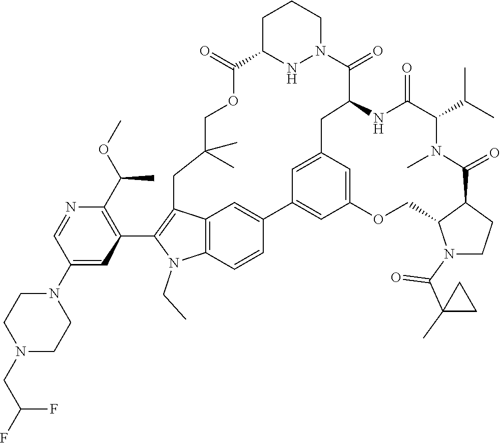 |
| A287 | 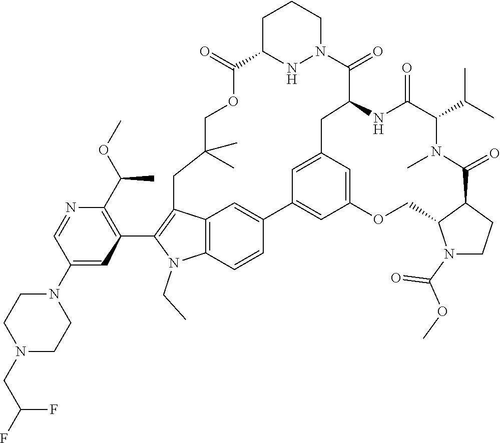 |
| A288 | 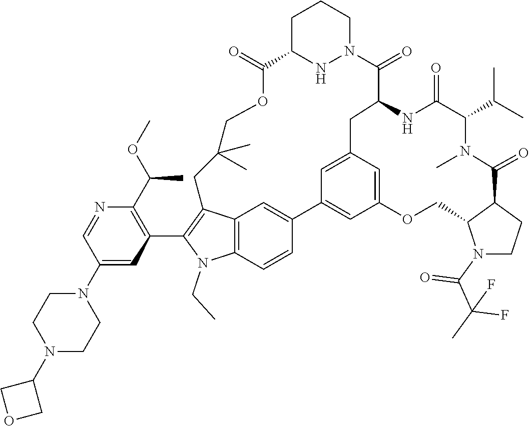 |
| A289 | 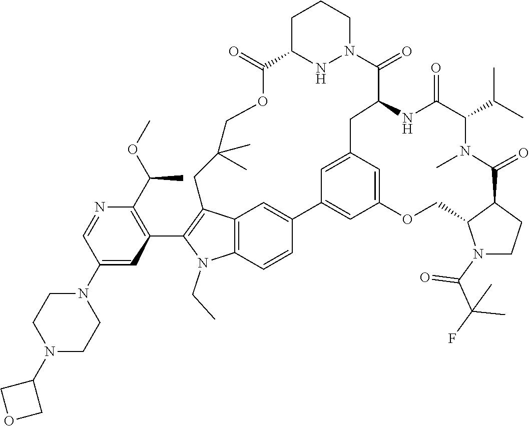 |
| A290 | 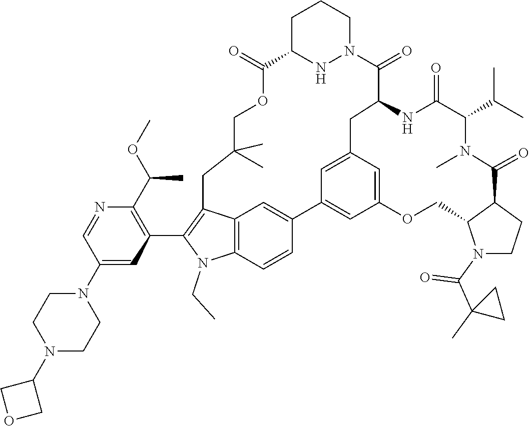 |
| A291 | 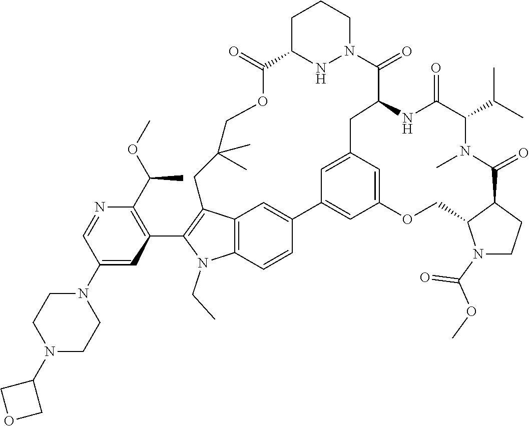 |
| A292 | 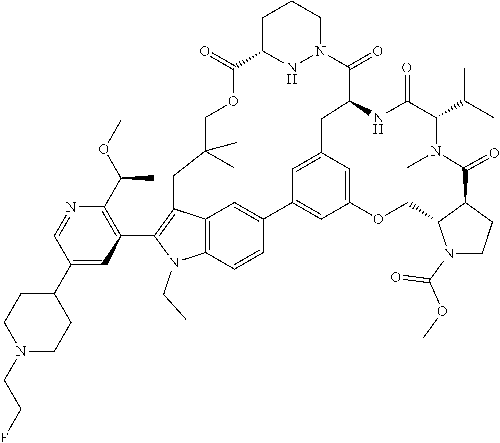 |
| A293 | 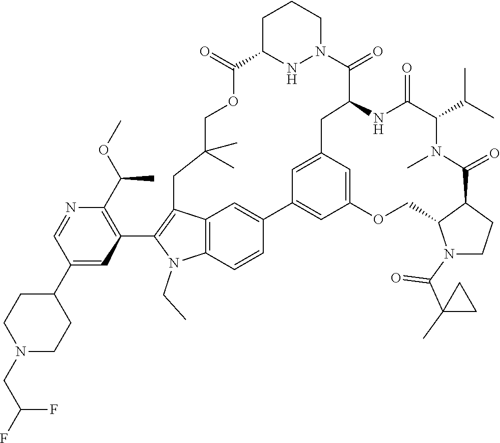 |
| A295 | 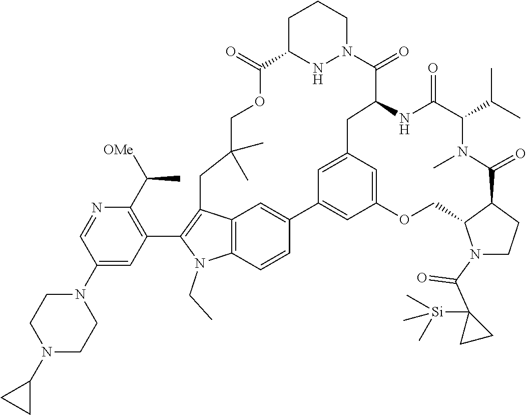 [SMILES: CCn1c(-c2cc(N3CCN(C4CC4)CC3)cnc2[C@H](C)OC)c2c3cc(ccc31)-c1cc3cc(c1)OC[C@@H]1[C@H](CCN1C(=O)C1([Si](C)(C)C)CC1)C(=O)N(C)[C@@H](C(C)C)C(=O)N[C@@H](C3)C(=O)N1CCC[C@H](N1)C(=O)OCC(C)(C)C2] |
| A296 | 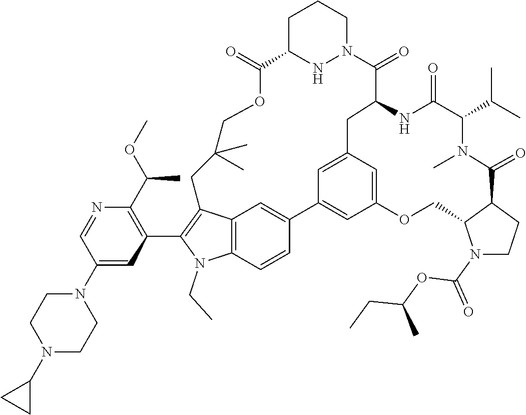 |
| A297 | 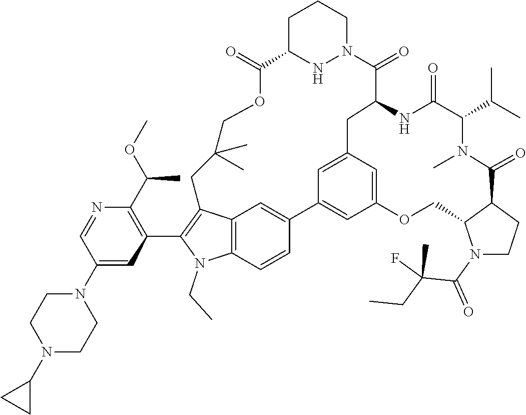 |
| A298 | 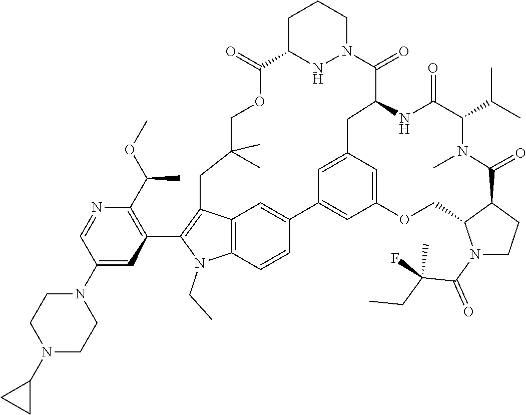 |
| A299 | 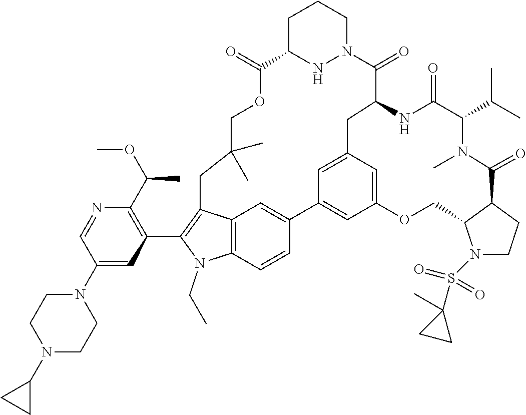 |
| A300 | 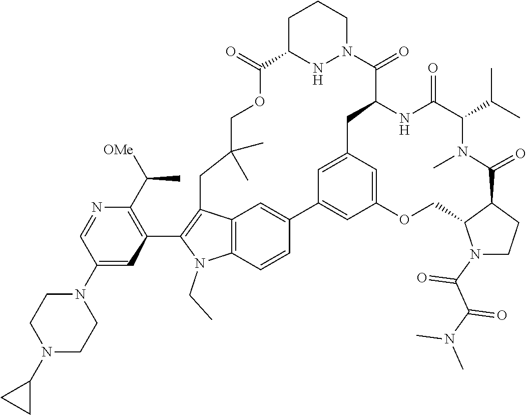 |
| A301 | 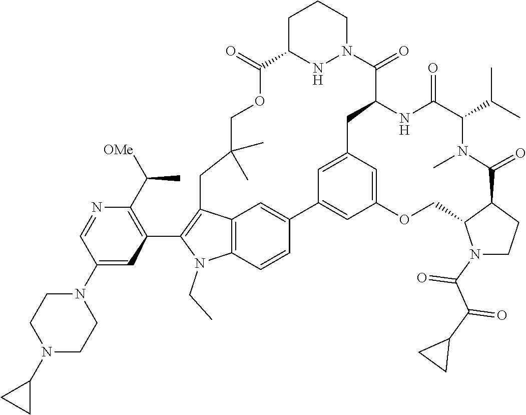 |
| A302 | 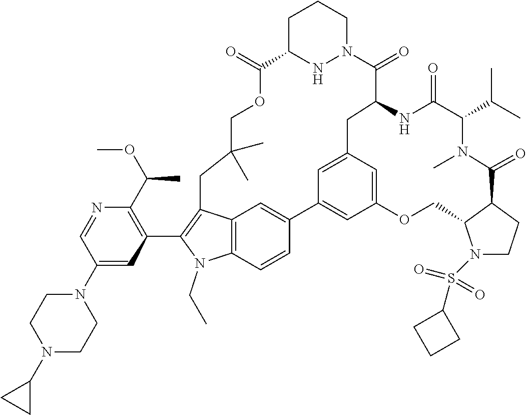 |
| A303 | 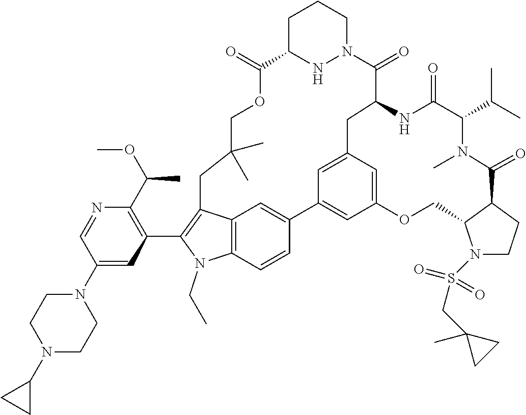 |
| A304 | 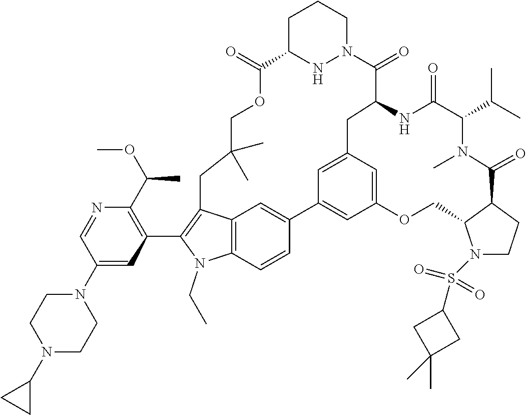 |
| A305 | 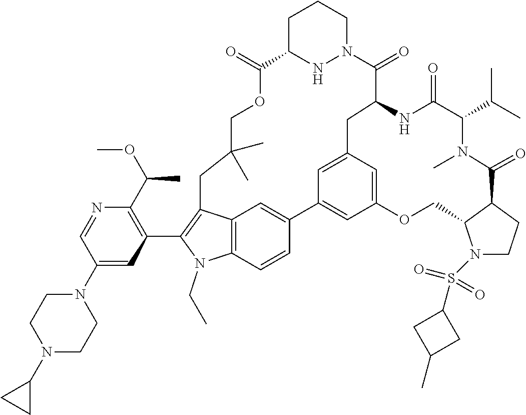 |
| A306 | 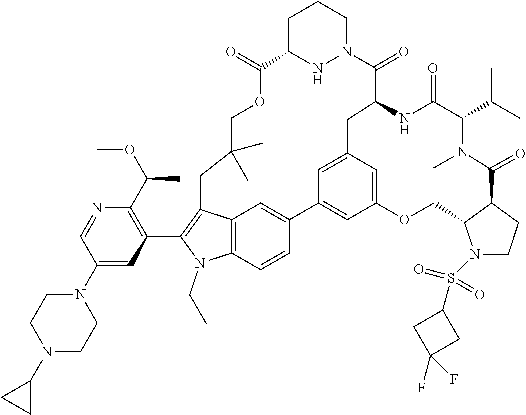 |
| A307 | 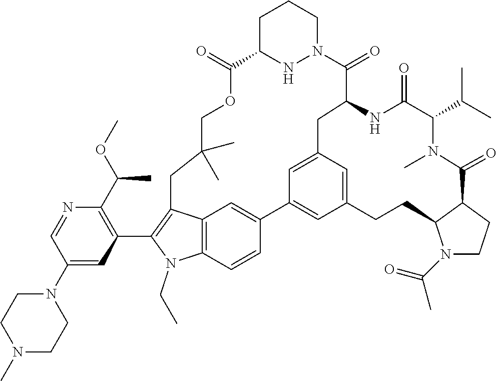 |
| A308 | 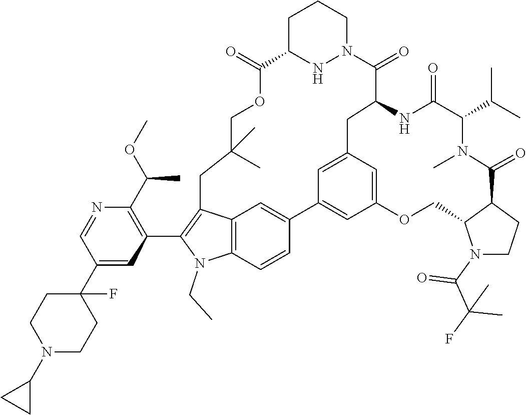 |
| A309 | 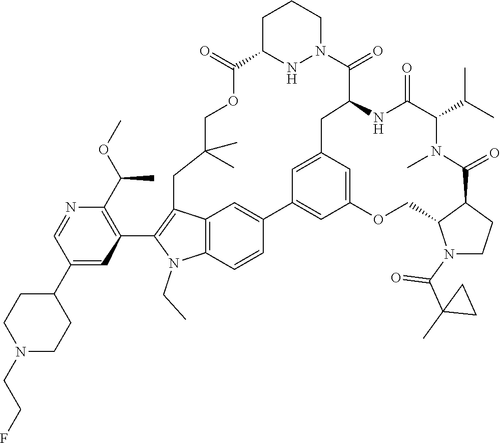 |
| A310 | 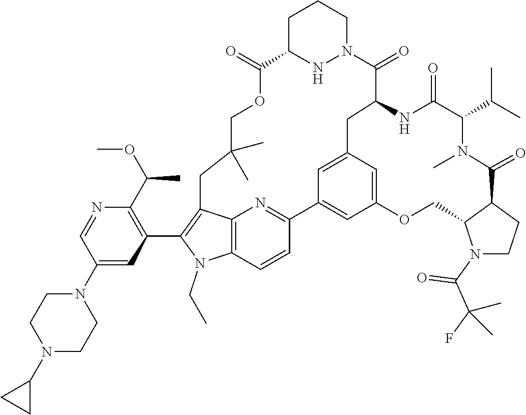 |
| A311 | 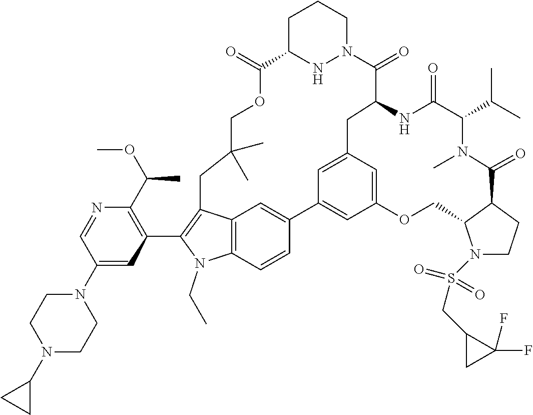 |
| A312 | 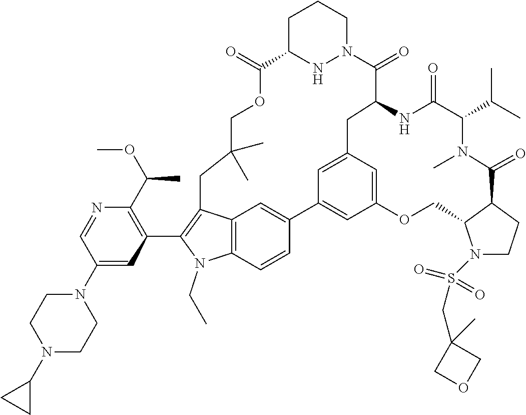 |
| A313 | 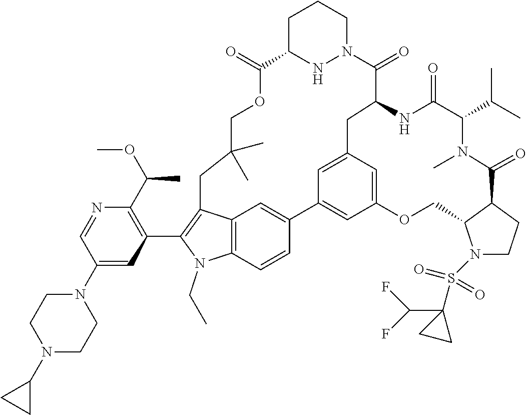 |
| A314 | 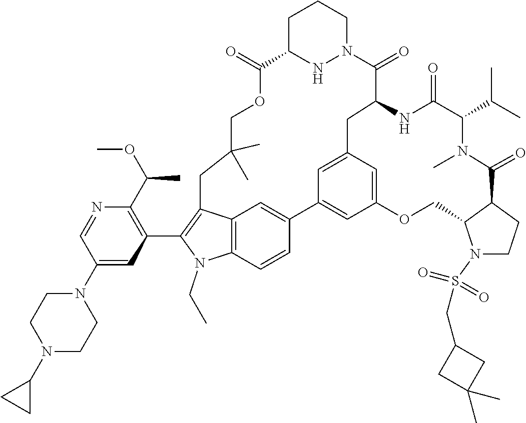 |
| A315 | 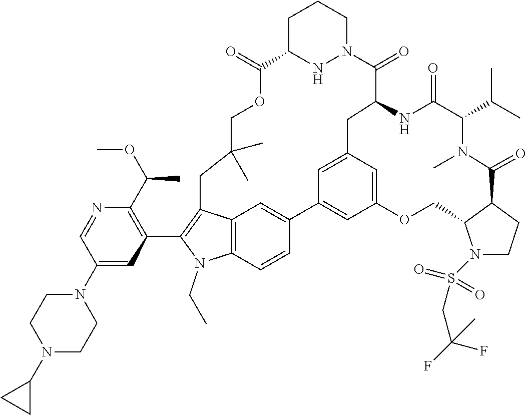 |
| A316 | 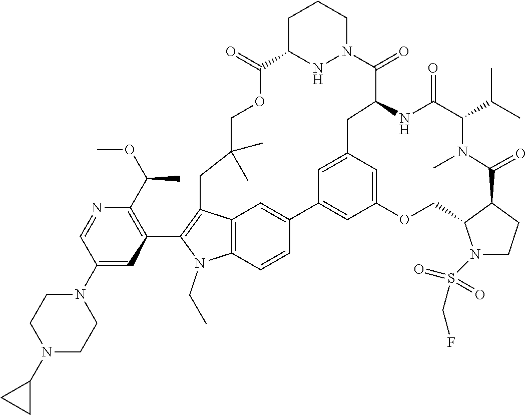 |
| A317 | 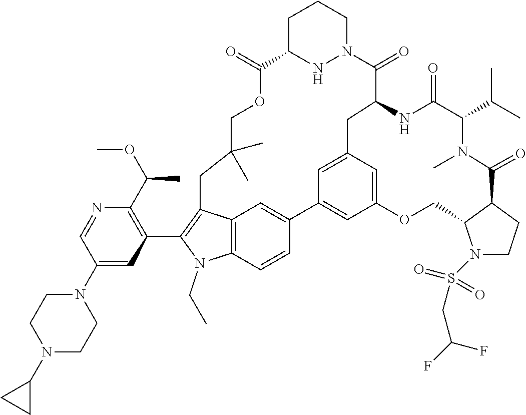 |
| A318 | 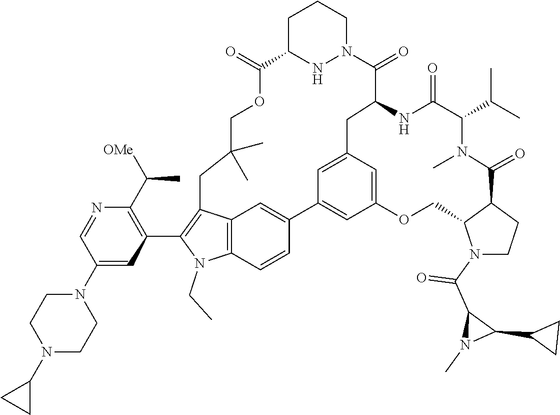 |
| A319 | 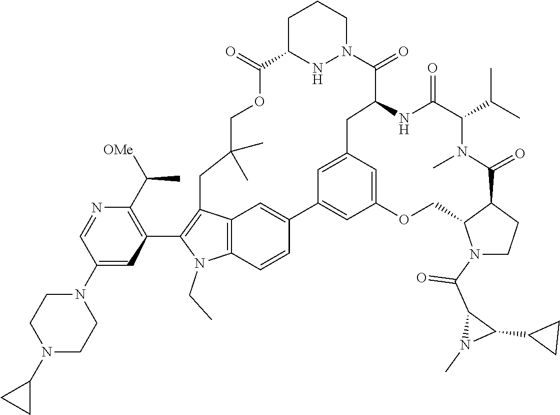 |
| A320 | 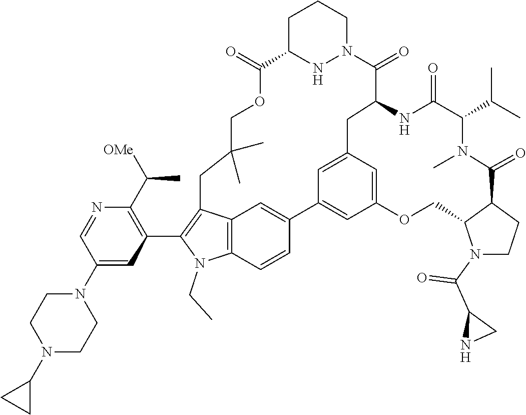 |
| A321 | 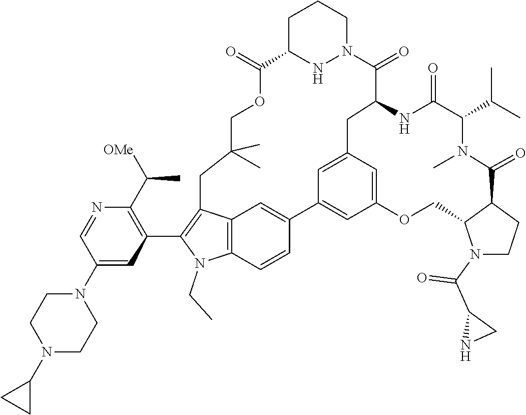 |
| A322 | 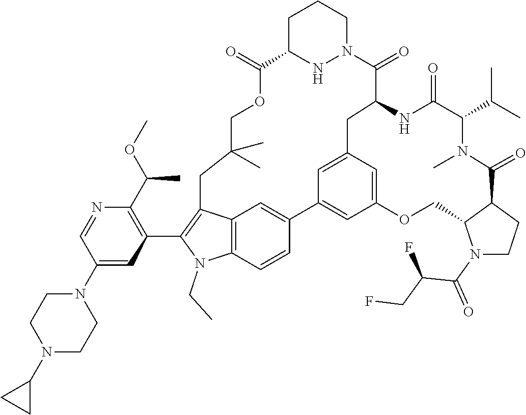 |
| A323 | 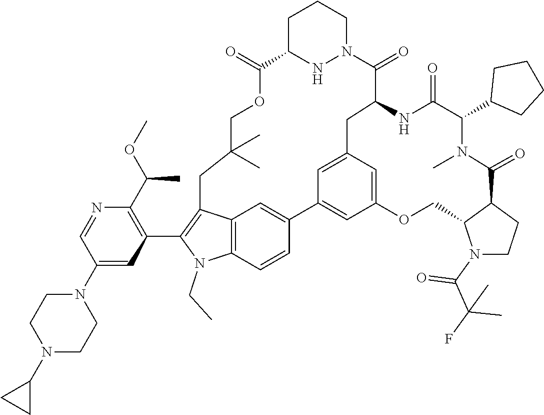 |
| A324 | 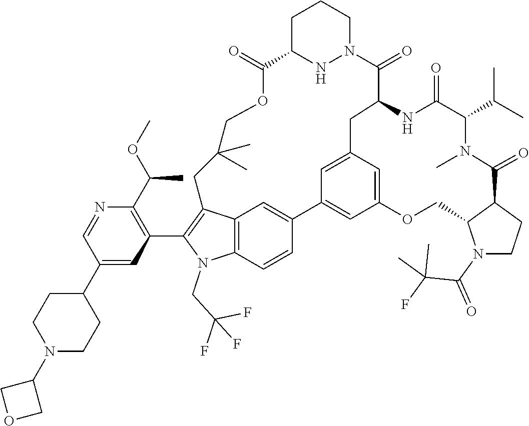 |
| A325 | 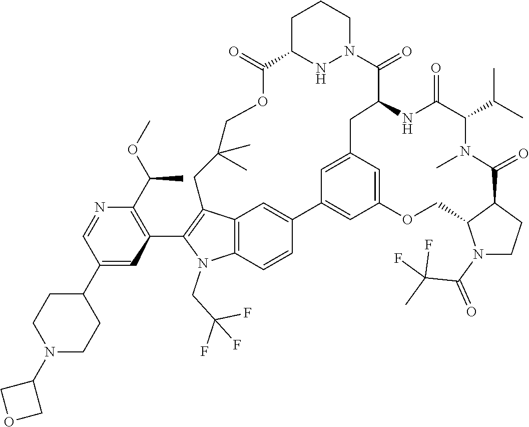 |
| A326 | 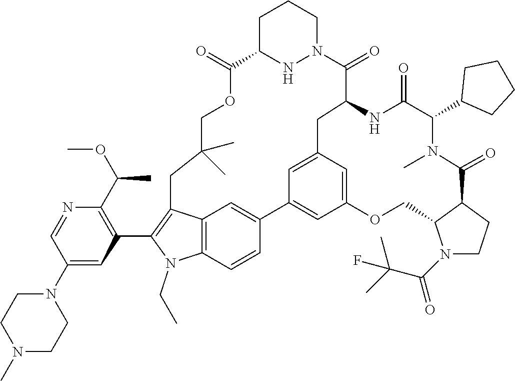 |
| A327 | 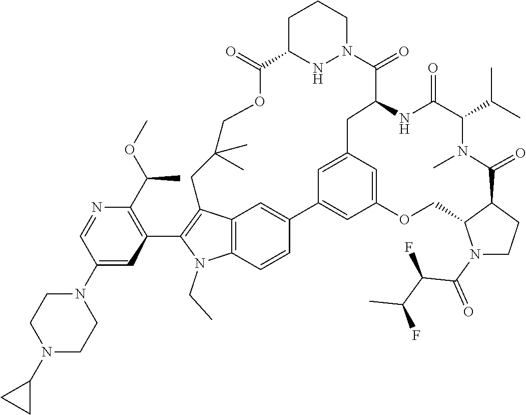 |
| A328 | 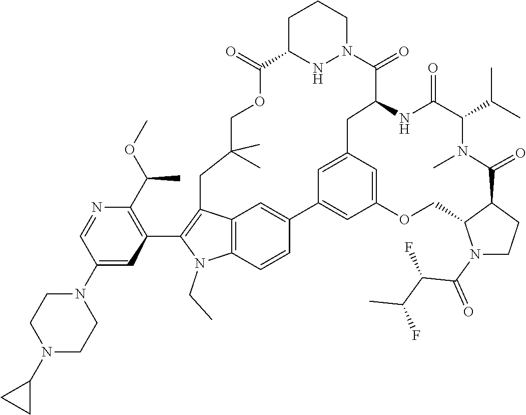 |
| A329 | 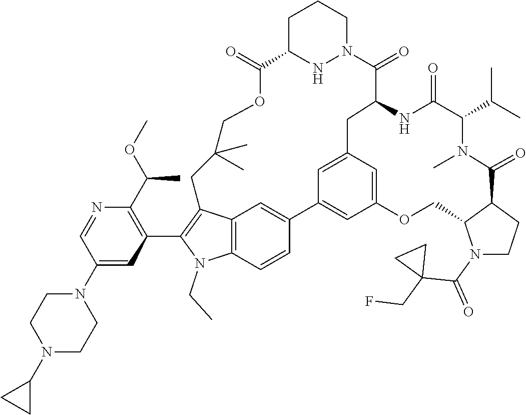 |
| A330 | 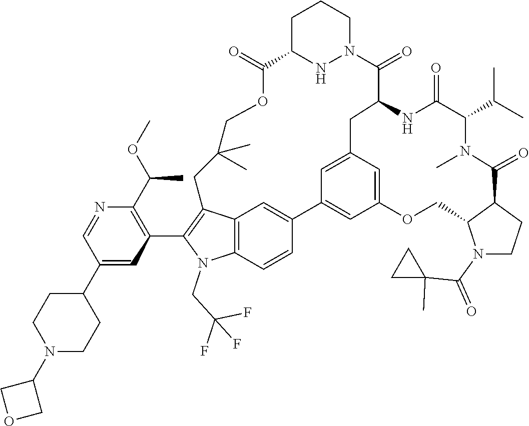 |
| A331 | 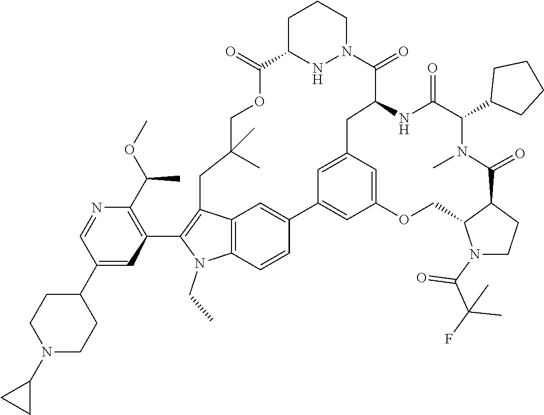 |
| A332 | 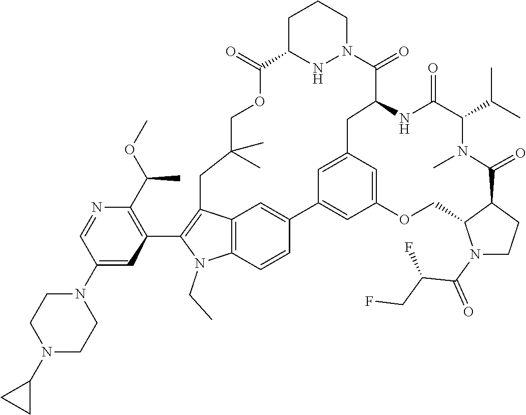 |
| A333 | 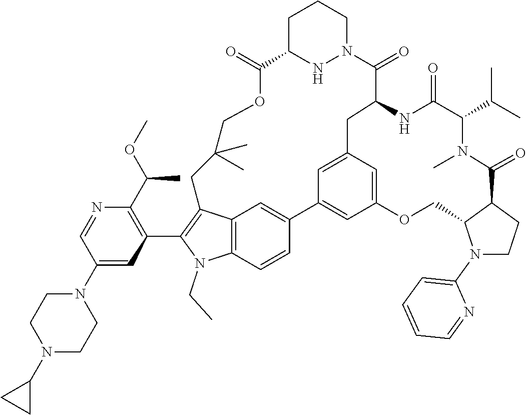 |
| A334 | 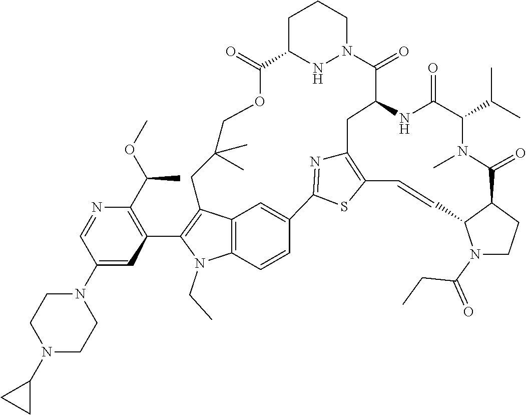 |
| A335 | 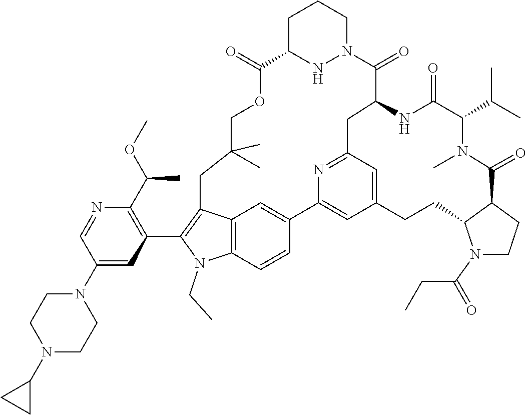 |
| A336 | 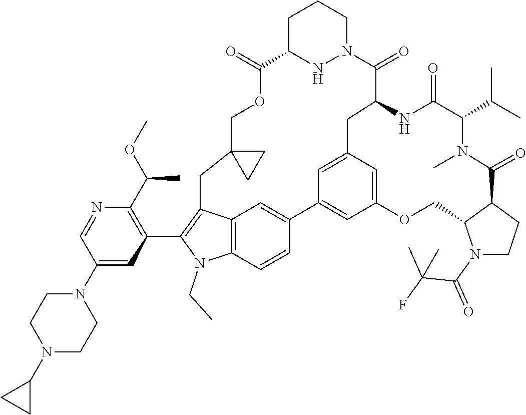 |
| A337 | 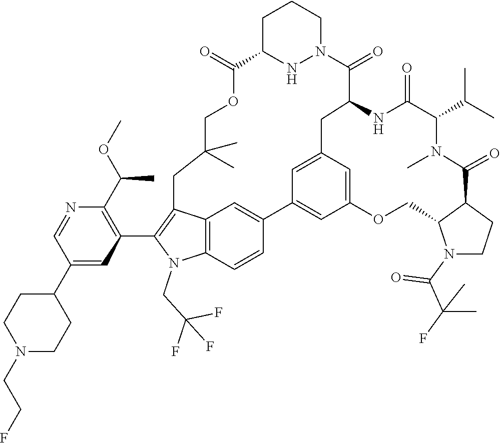 |
| A338 | 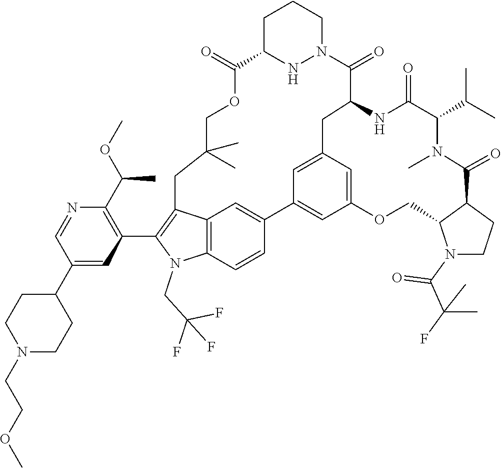 |
| A340 | 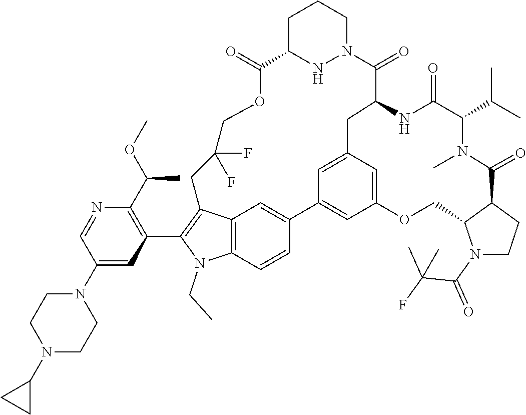 |
| A341 | 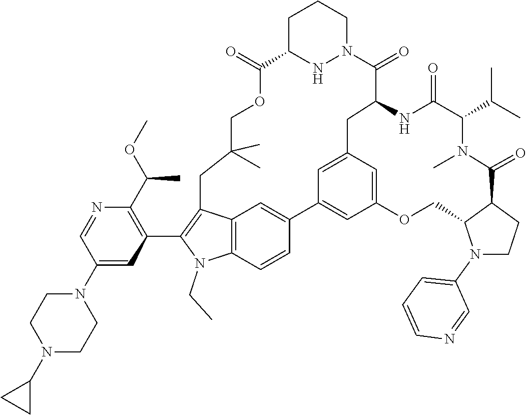 |
| A342 | 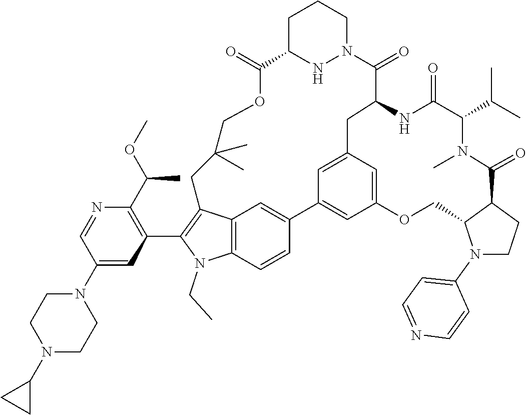 |
| A343 | 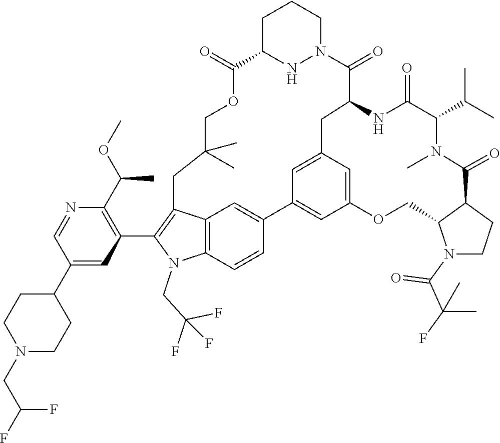 |
| A344 | 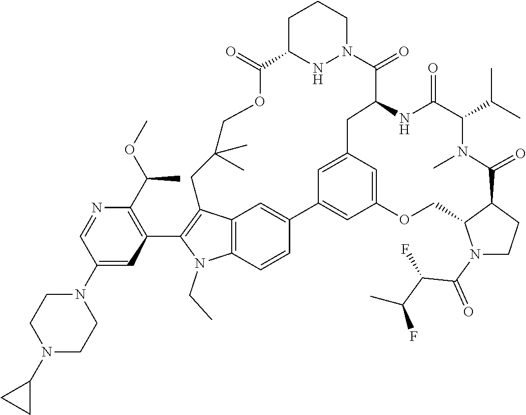 |
| A345 | 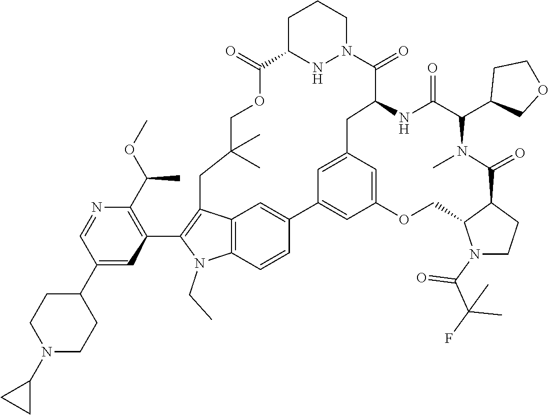 |
| A346 | 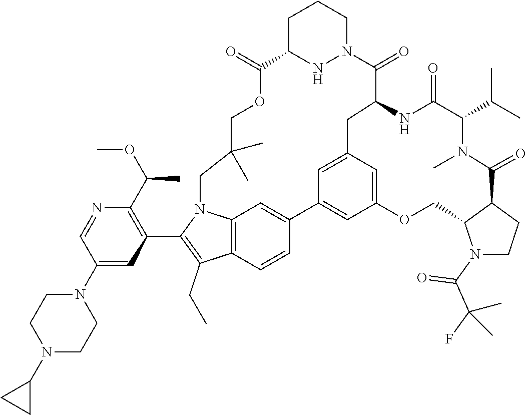 |
| A347 | 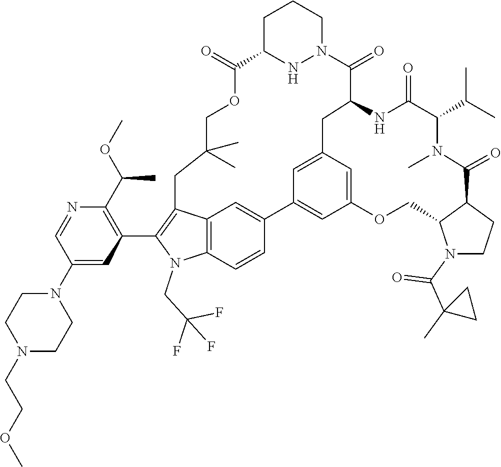 |
| A348 | 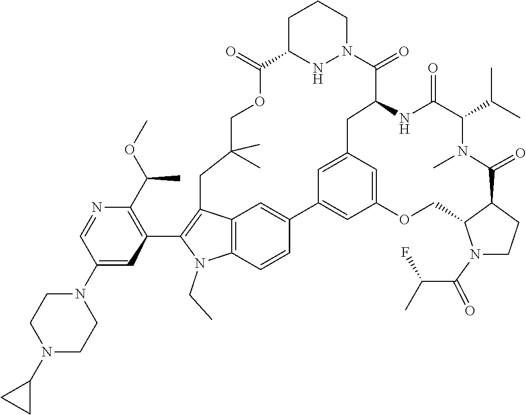 |
| A349 | 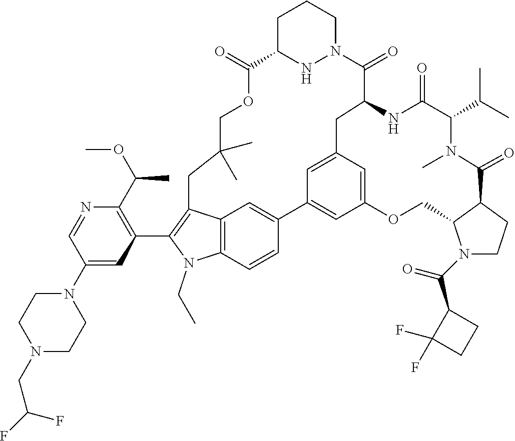 |
| A350 | 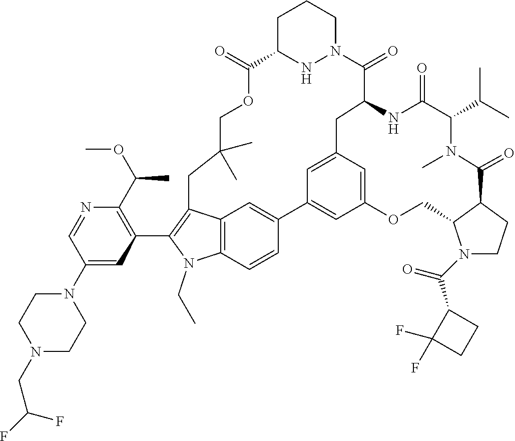 |
| A351 | 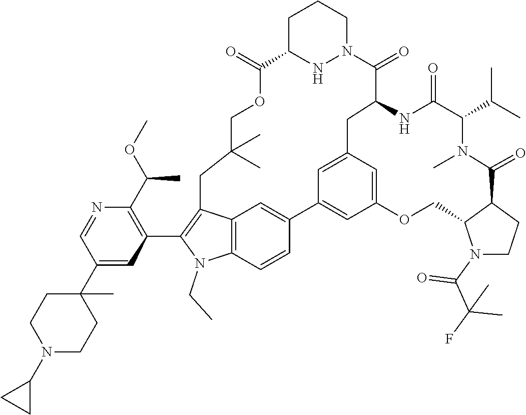 |
| A352 | 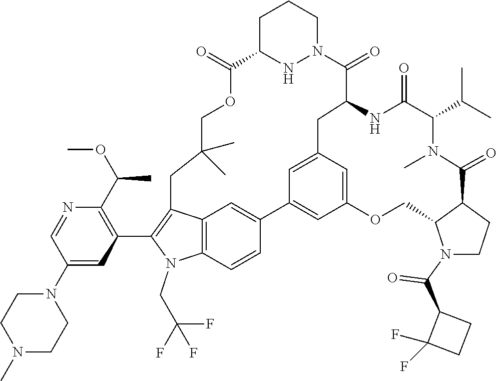 |
| A353 | 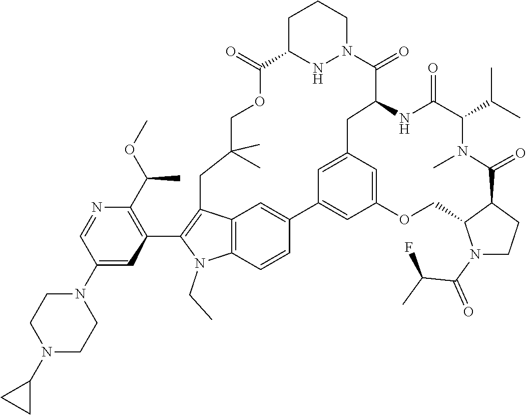 |
| A354 | 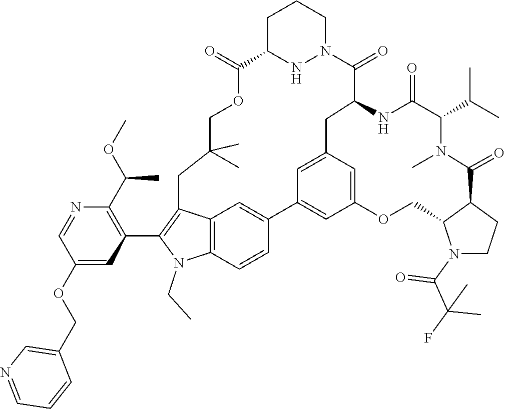 |
| A355 | 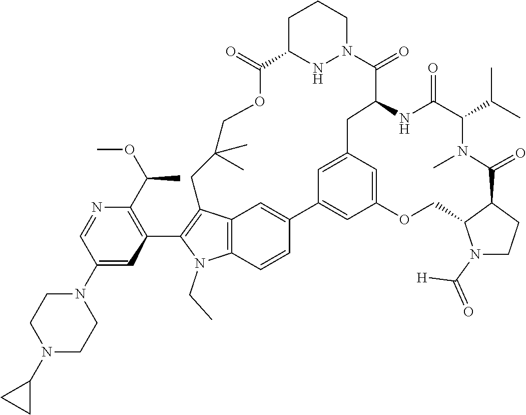 |
| A356 | 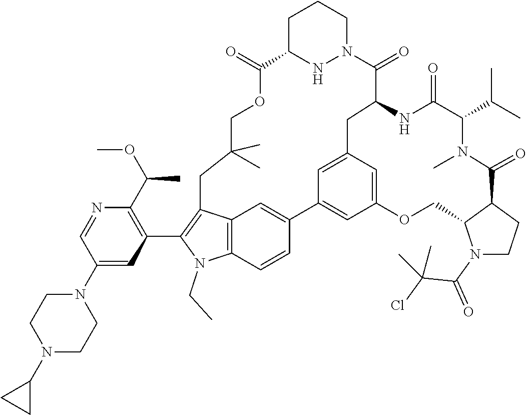 |
| A357 | 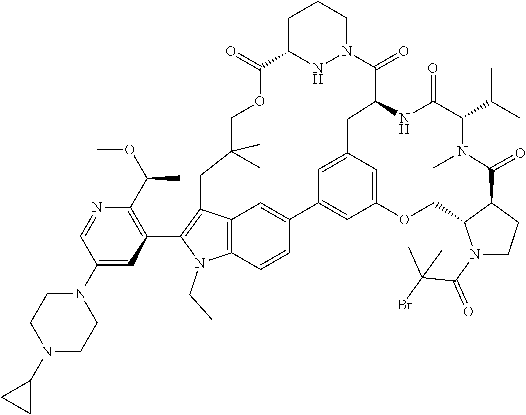 |
| A358 | 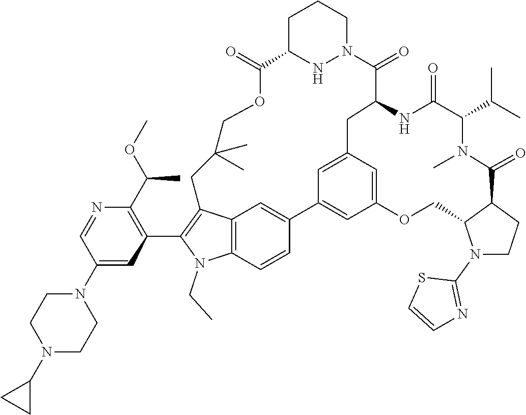 |
| A359 | 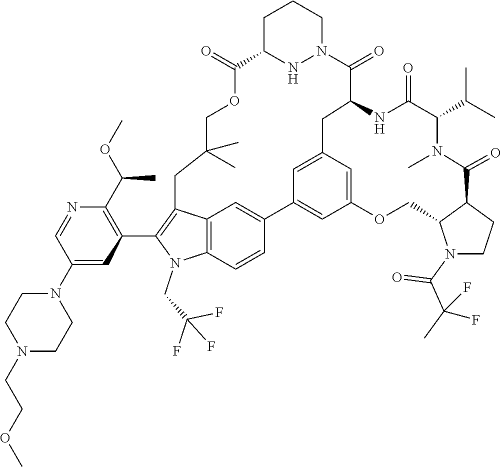 |
| A360 | 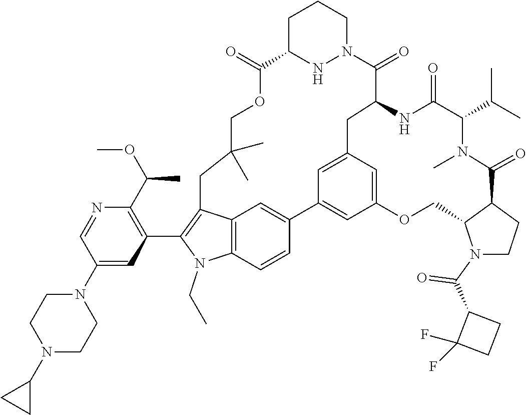 |
| A361 | 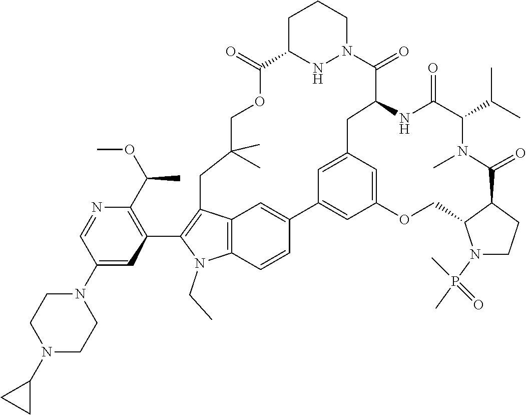 |
| A362 | 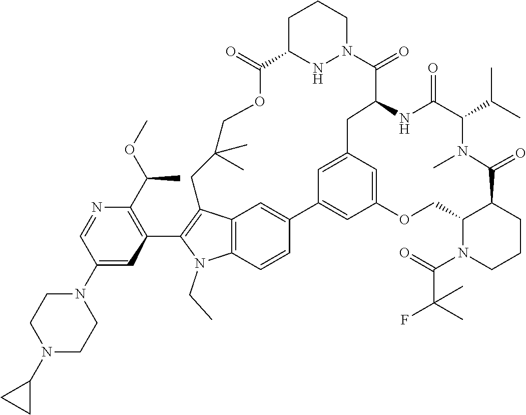 |
| A363 | 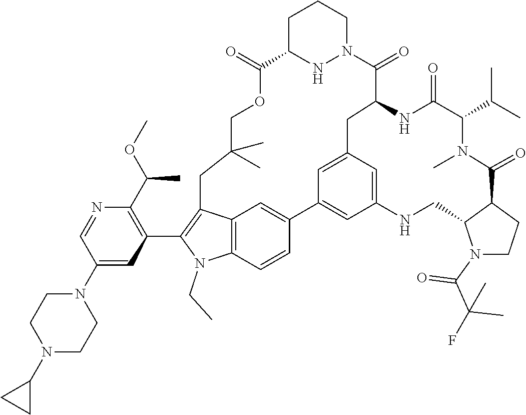 |
| A364 | 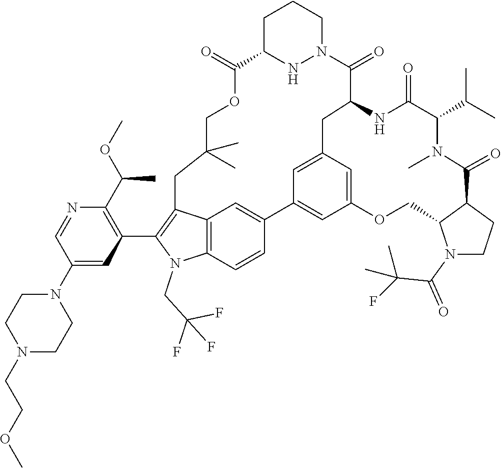 |
| A365 | 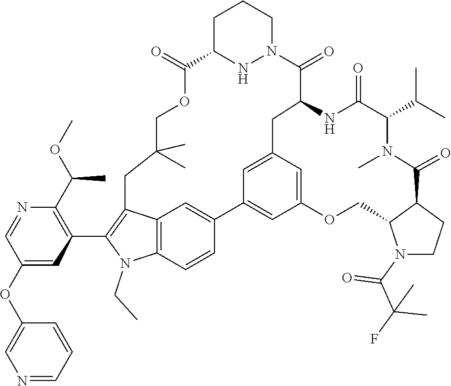 |
| A366 | 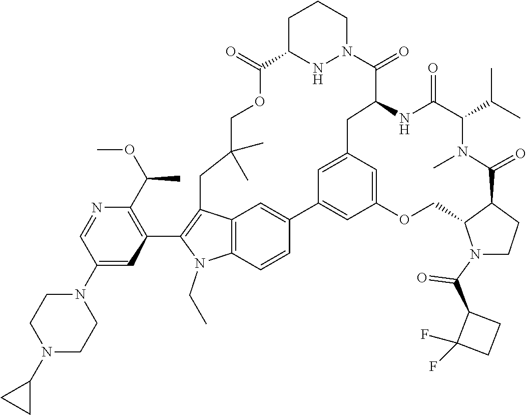 |
| A367 | 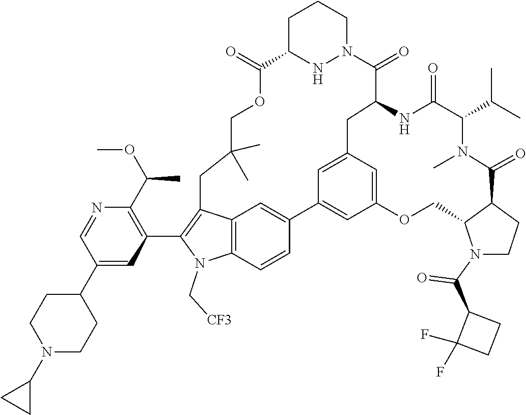 |
| A368 | 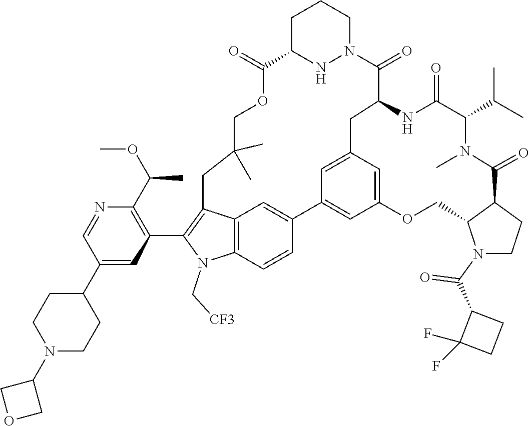 |
| A369 | 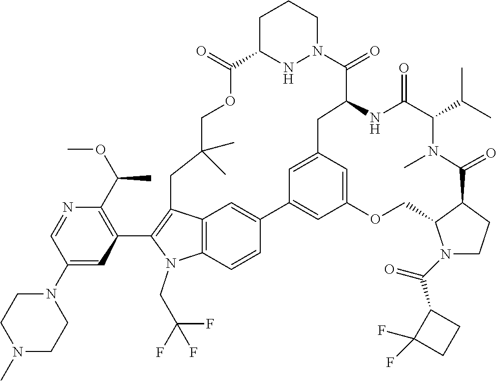 |
| A370 | 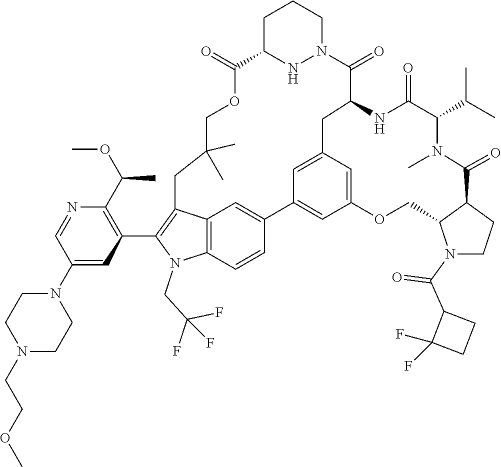 |
| A371 | 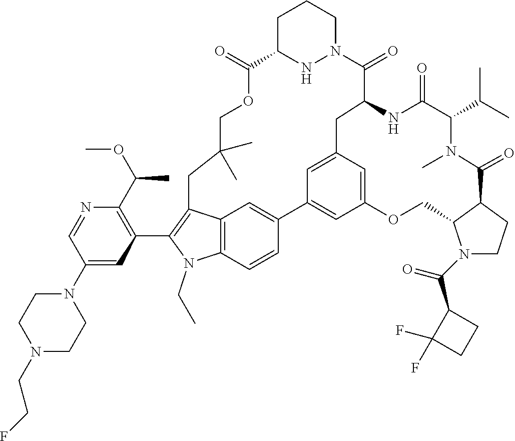 |
| A372 | 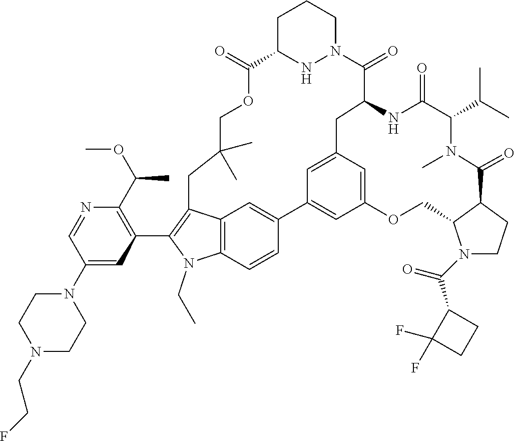 |
| A373 | 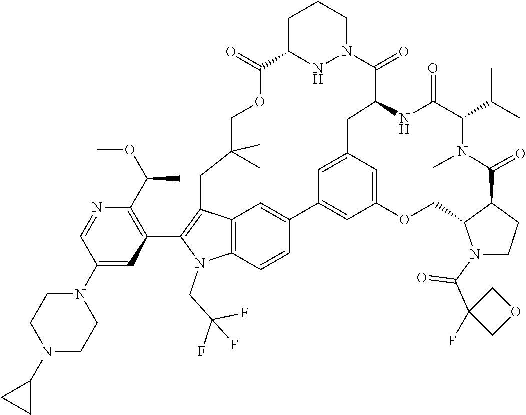 |
| A374 | 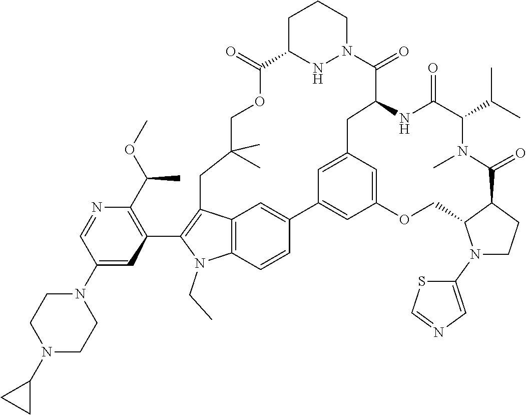 |
| A375 | 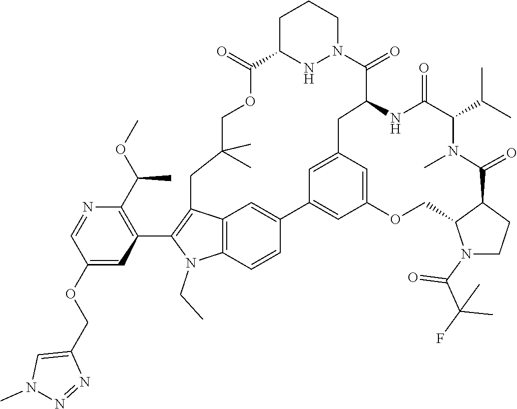 |
| A376 | 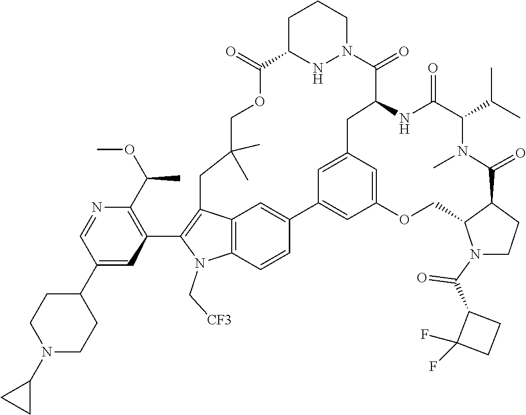 |
| A377 | 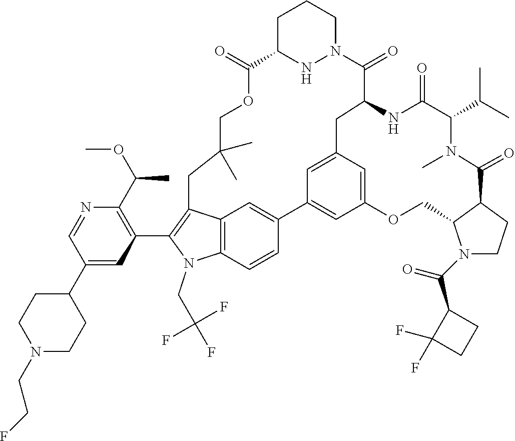 |
| A378 | 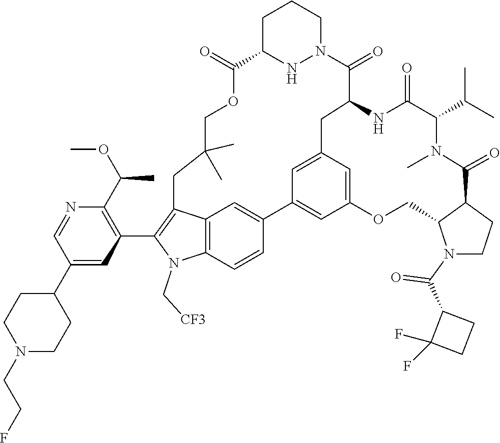 |
| A379 | 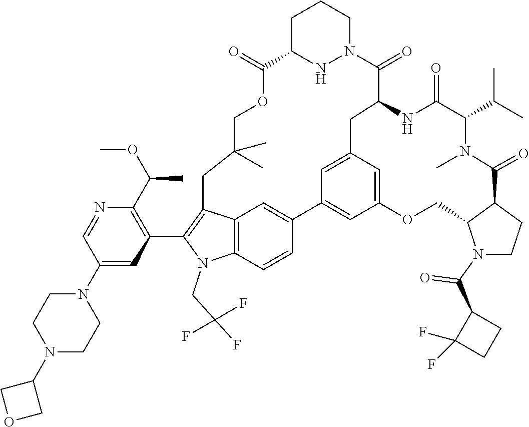 |
| A380 | 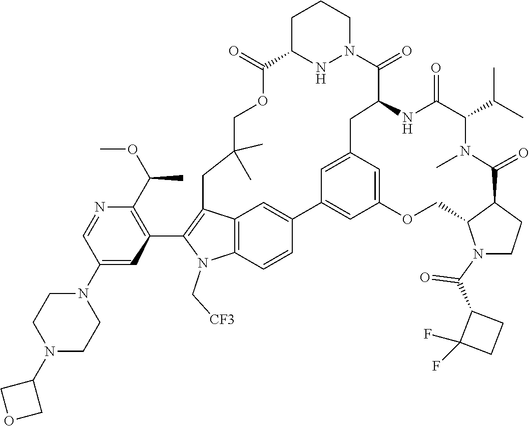 |
| A381 | 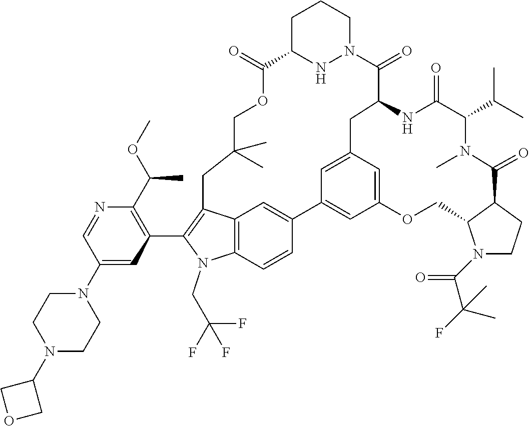 |
| A382 | 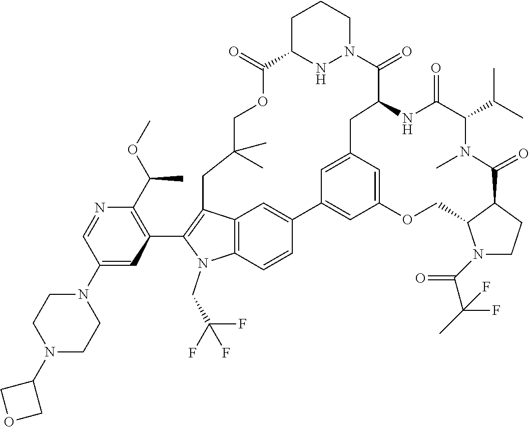 |
| A383 | 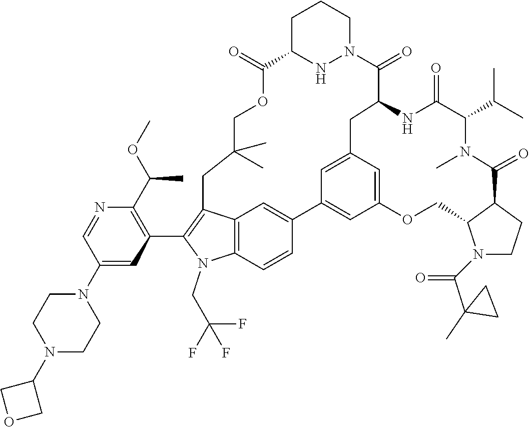 |
| A384 | 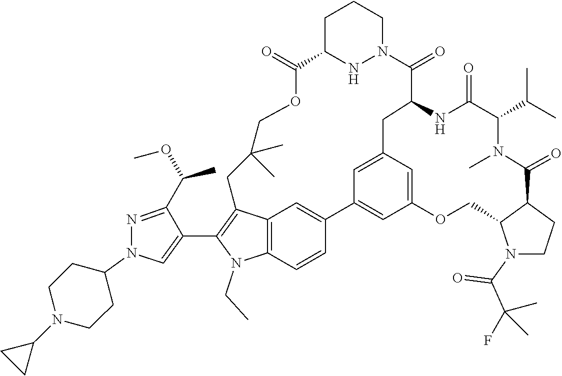 |
| A385 | 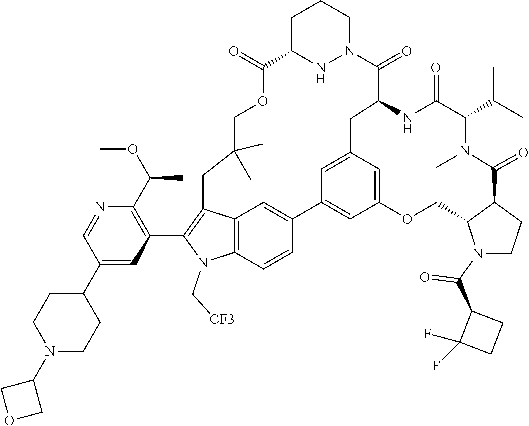 |
| A386 | 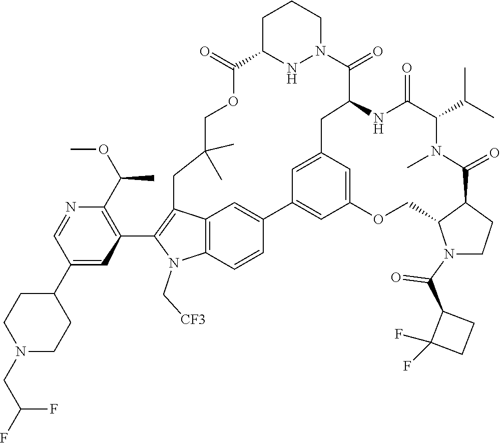 |
| A387 | 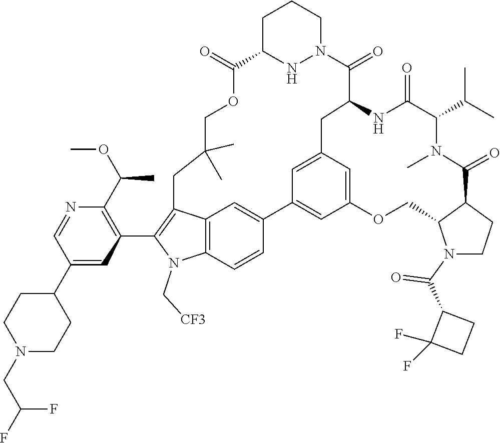 |
| A388 | 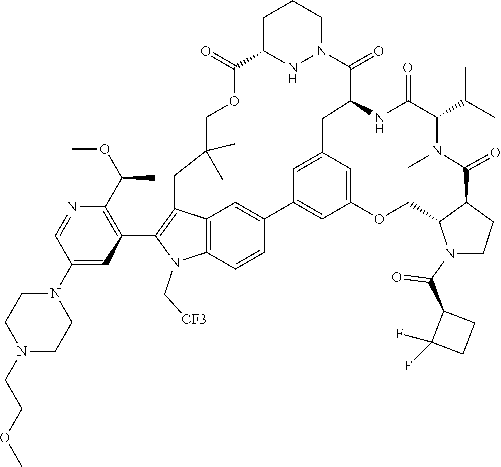 |
| A389 | 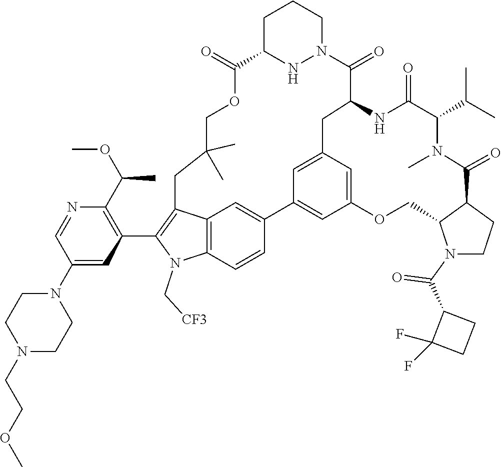 |
| A390 | 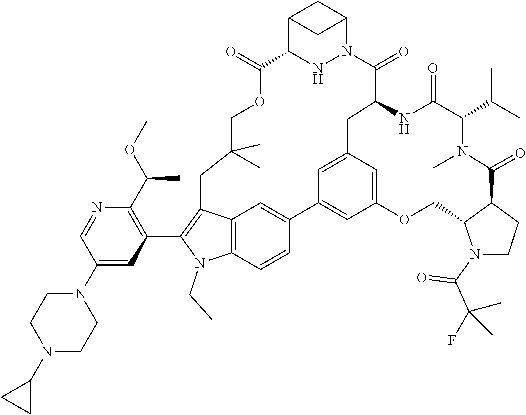 |
| A391 | 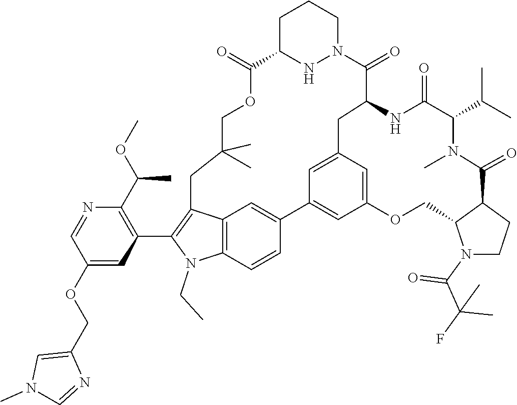 |
| A392 | 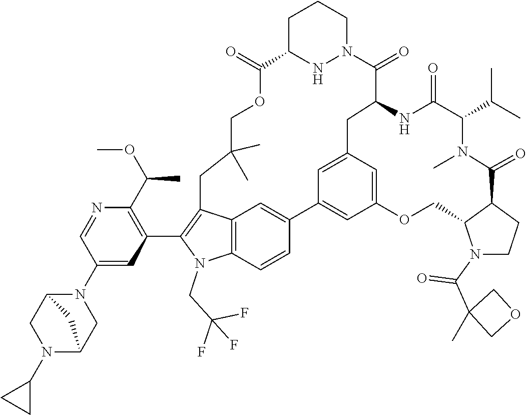 |
| A393 | 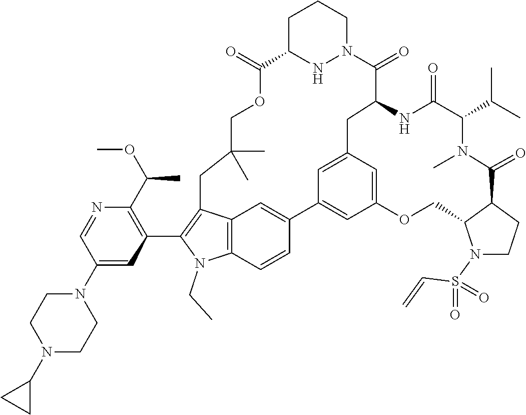 |
| A394 | 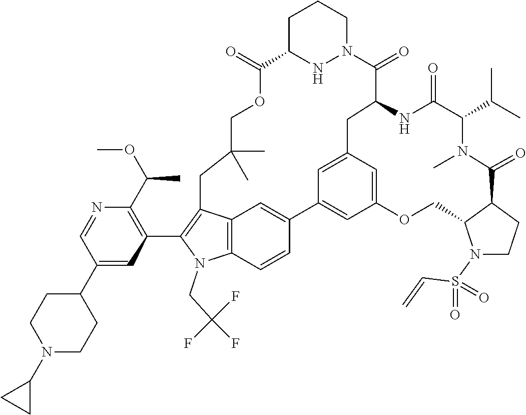 |
| A395 | 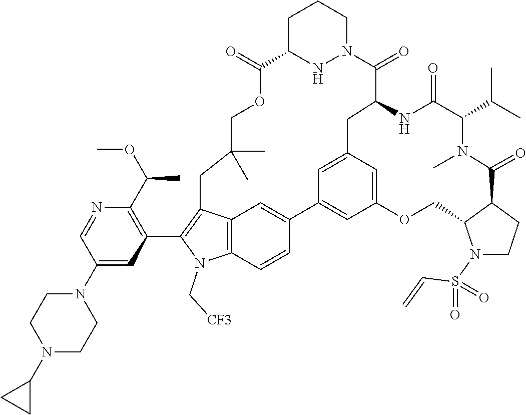 |
| A396 | 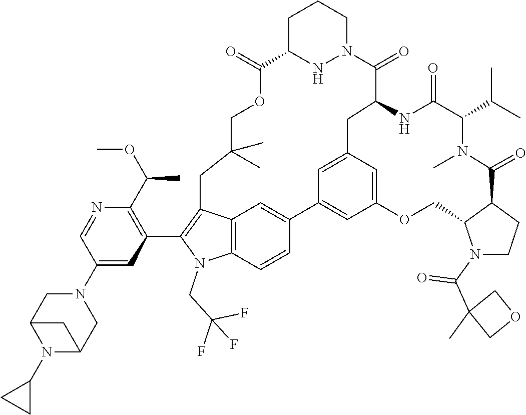 |
| A397 | 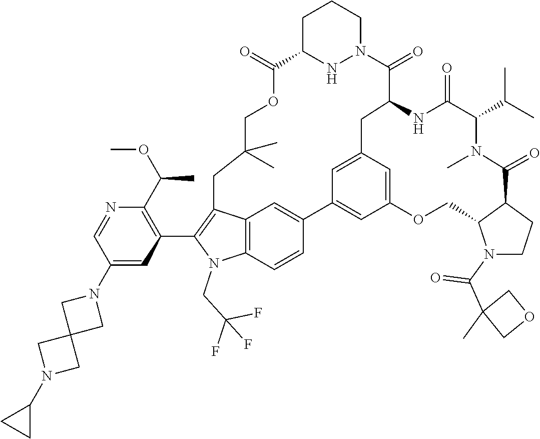 |
| A398 | 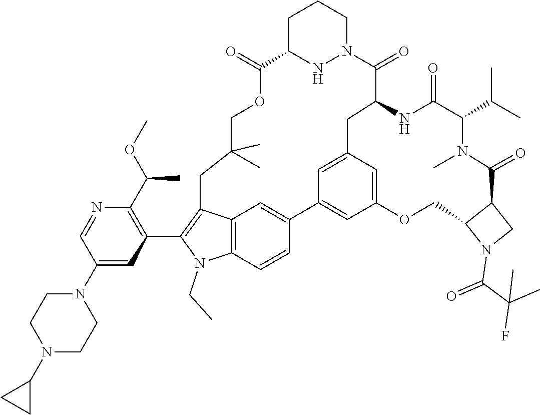 |
| A399 | 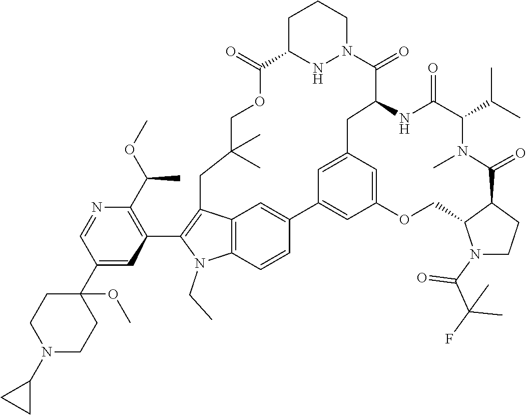 |
| A400 | 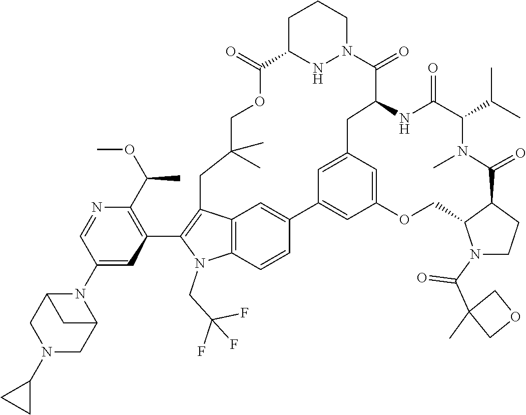 |
| A401 | 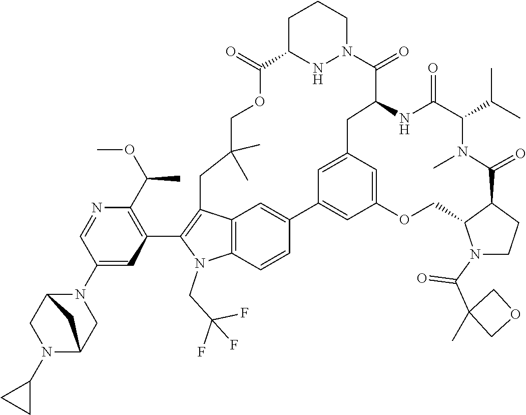 |
| A402 | 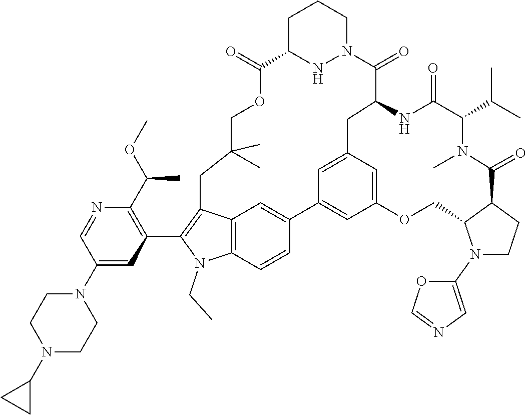 |
| A403 | 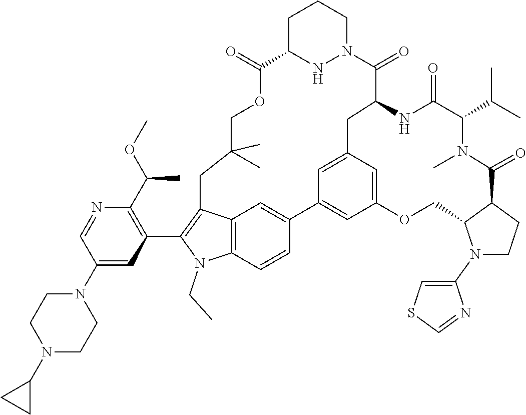 |
| A404 | 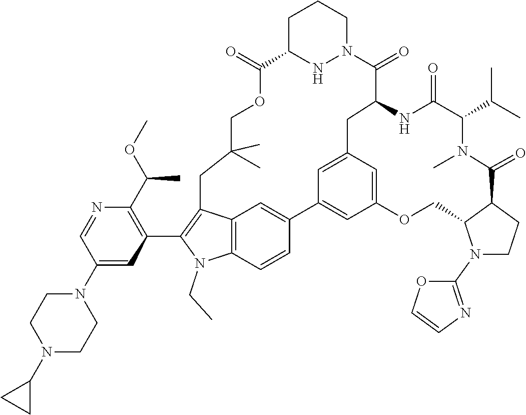 |
| A405 | 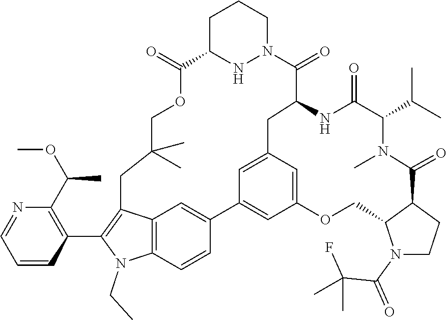 |
| A406 | 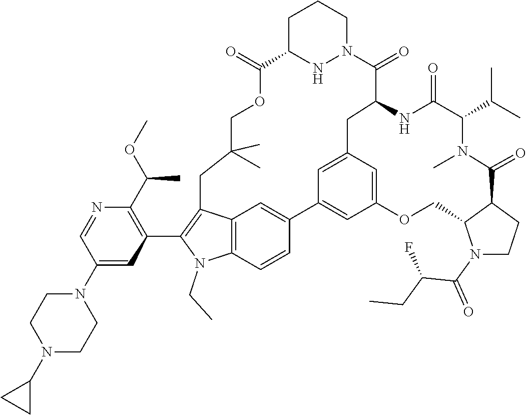 |
| A407 | 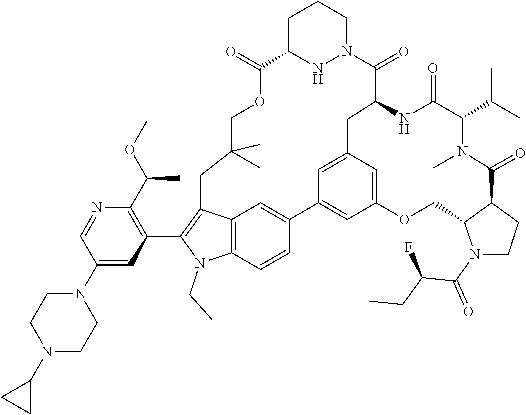 |
| A408 | 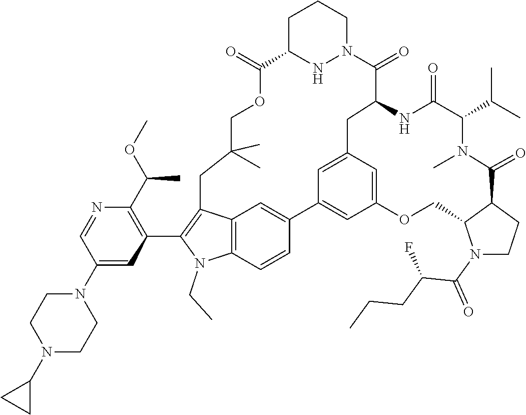 |
| A409 | 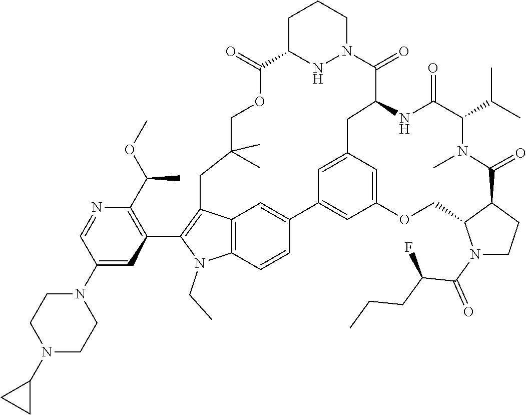 |
| A410 | 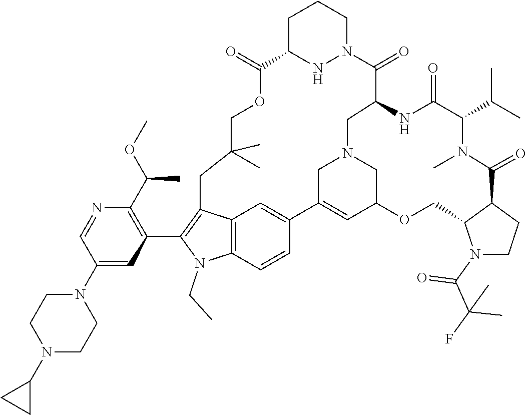 |
| A411 | 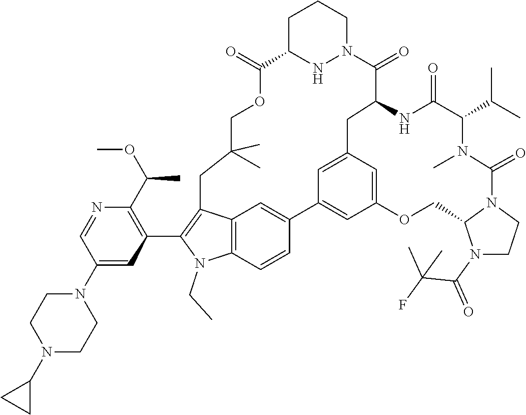 |
| A412 | 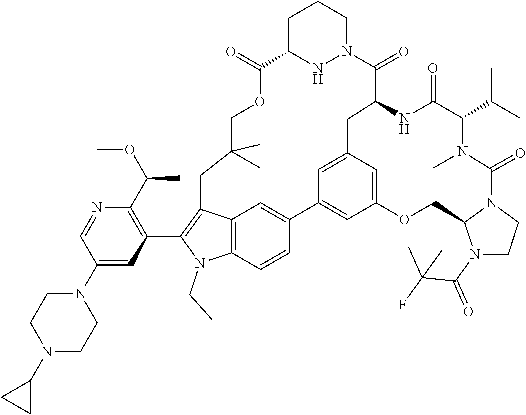 |
| A413 | 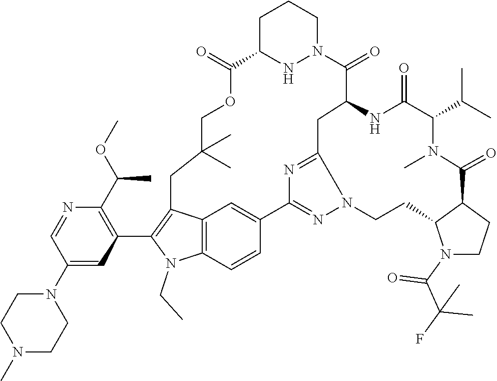 |
| A415 | 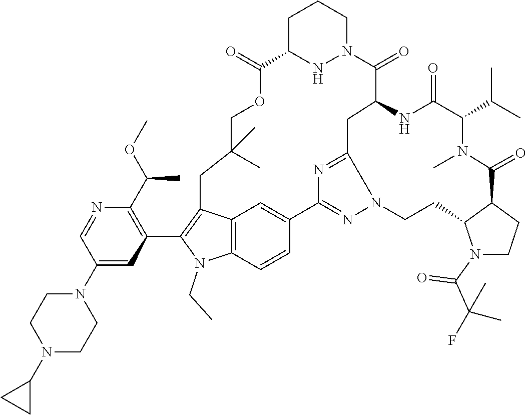 |
| A416 | 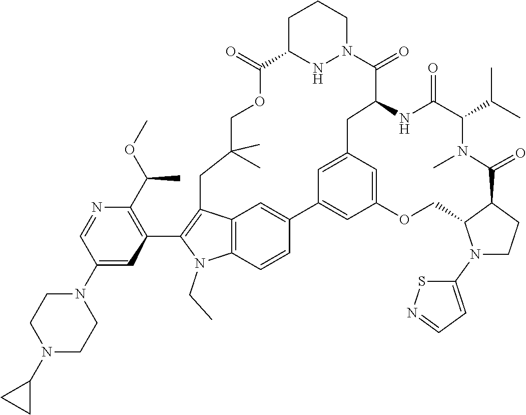 |
| A418 | 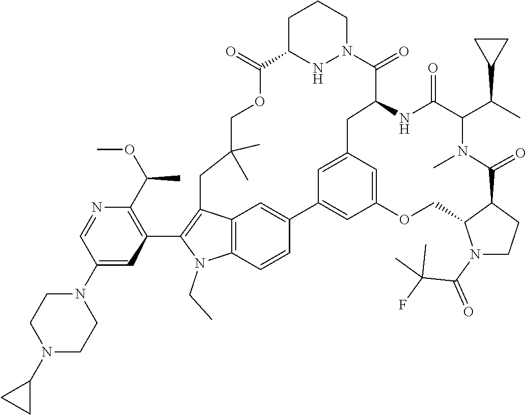 |
| A419 | 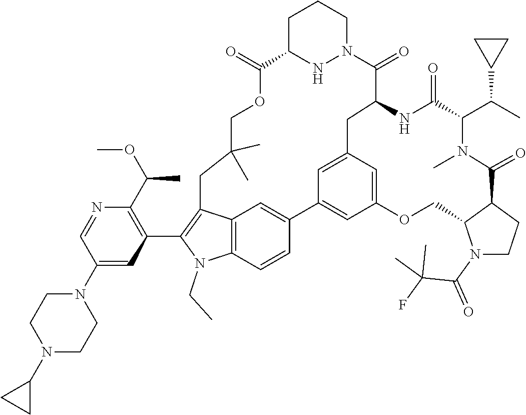 |
| A420 | 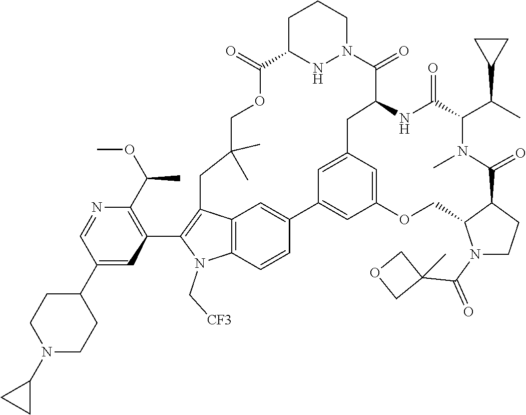 |
| A421 | 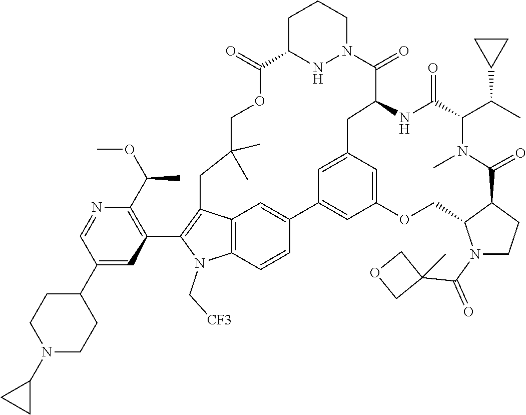 |
| A423 | 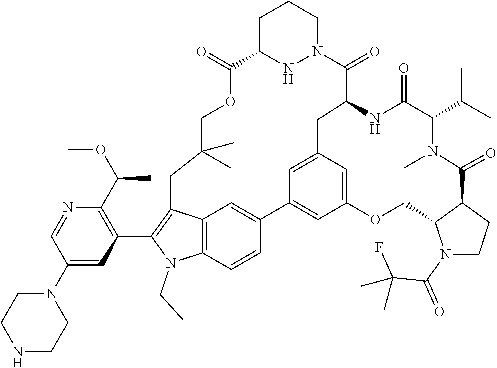 |
| A424 | 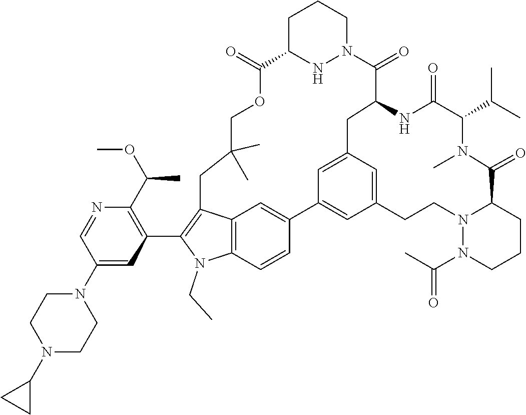 |
| A425 | 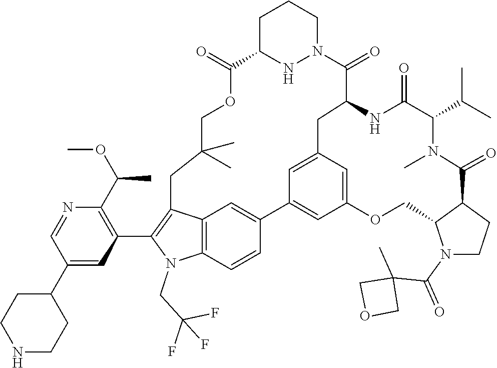 |
| A426 | 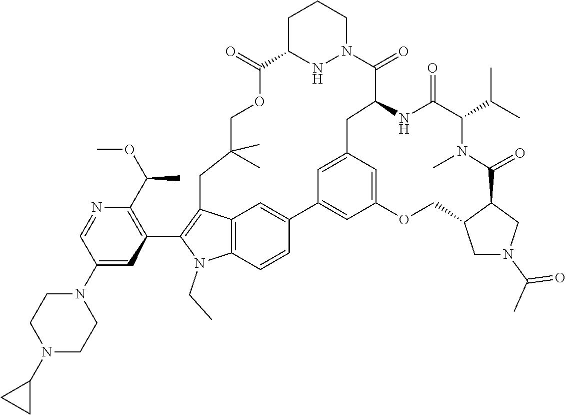 |
| A427 | 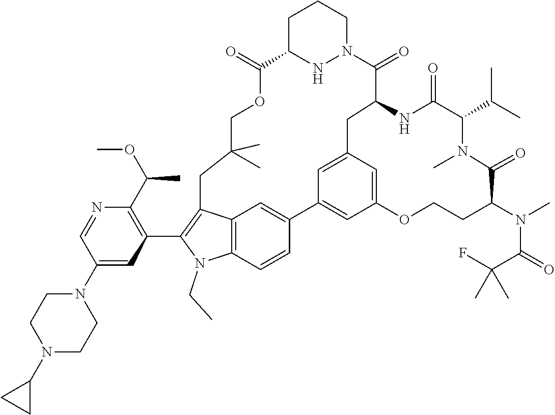 |
| A428 | 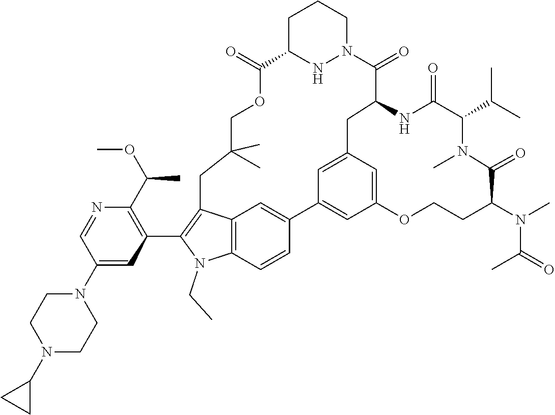 |
| A429 | 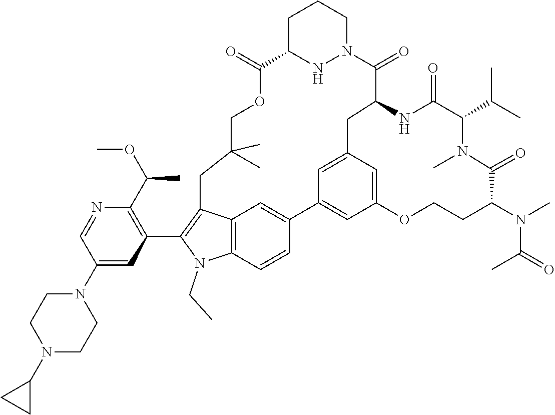 |
| A432 | 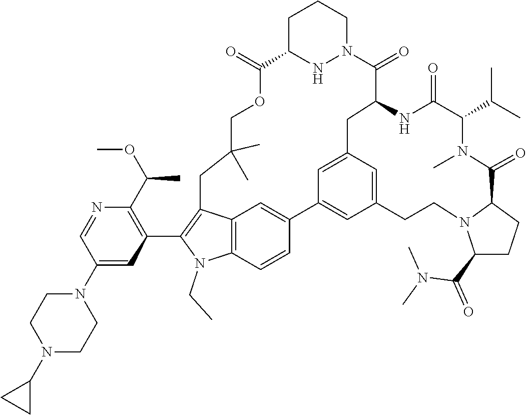 |
| A433 | 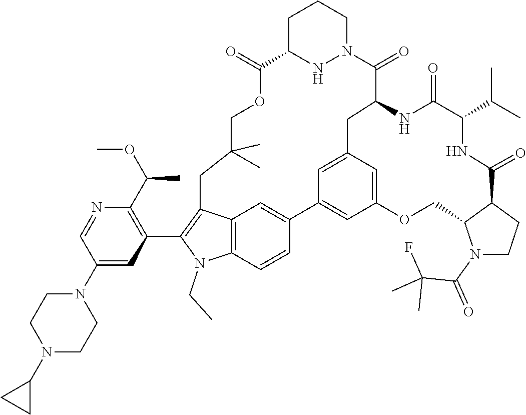 |
| A434 | 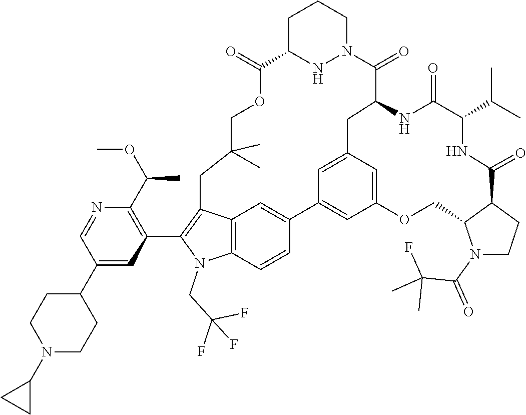 |
| A442 | 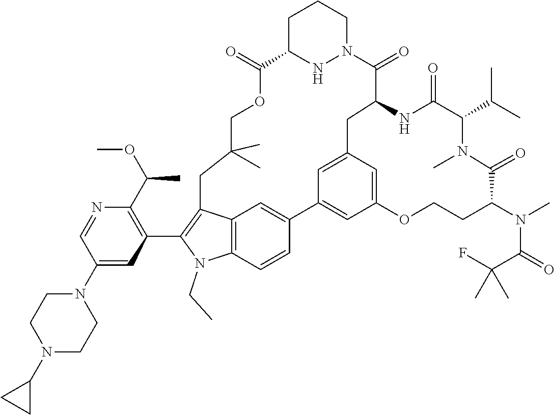 |
| A443 | 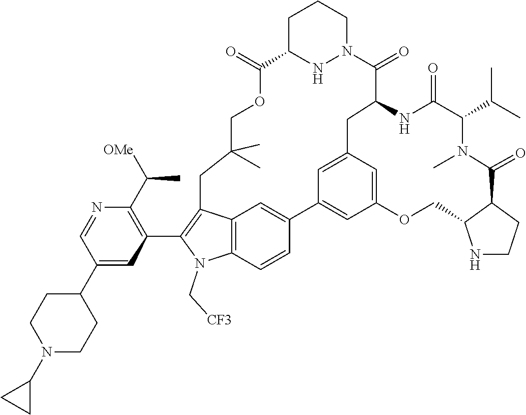 |
| A444 | 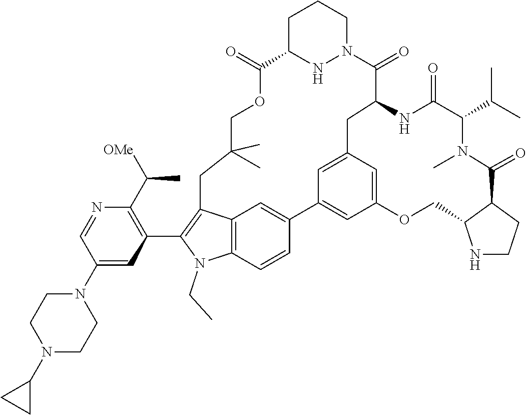 |
| A448 | 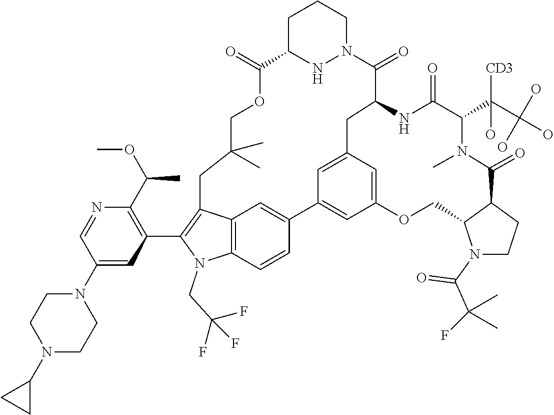 |
| A450 | 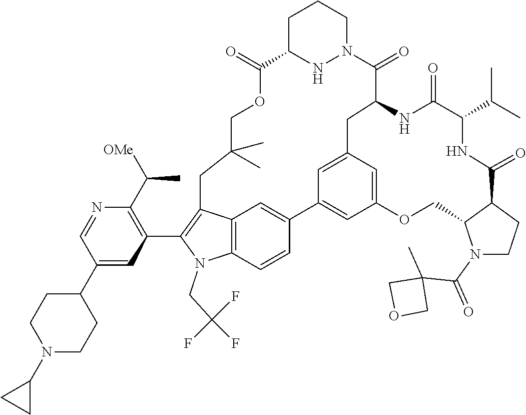 |

Note that some compounds are shown with bonds as flat or wedged. In some instances, the relative stereochemistry of stereoisomers has been determined; in some instances, the absolute stereochemistry has been determined. In some instances, a single Example number corresponds to a mixture of stereoisomers. All stereoisomers of the compounds of the foregoing table are contemplated by the present invention. In particular embodiments, an atropisomer of a compound of the foregoing table is contemplated. Brackets are to be ignored.

TABLE 2
Certain Compounds of the Present Invention

| | |
|---|---|
| 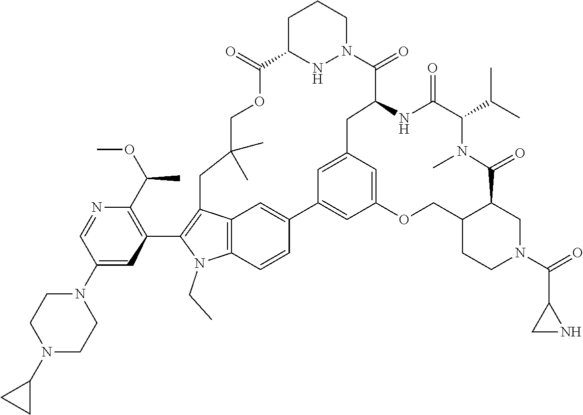 | B1 |
| [symbol] | |
| 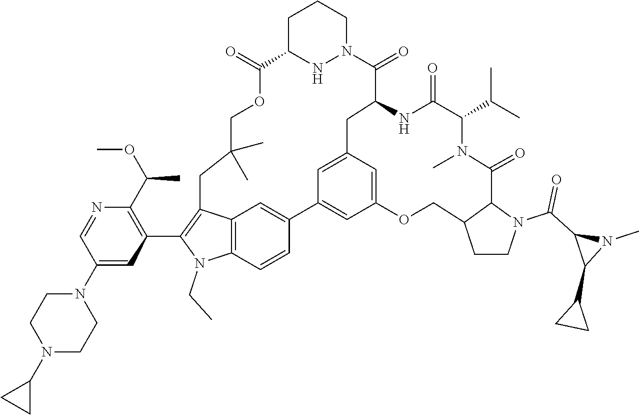 | B2 |
| 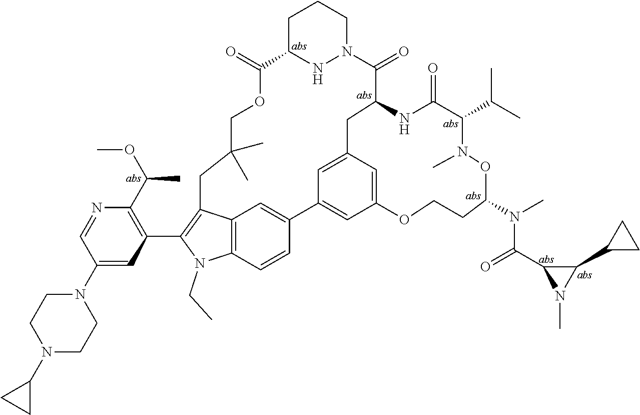 | B3 |
| 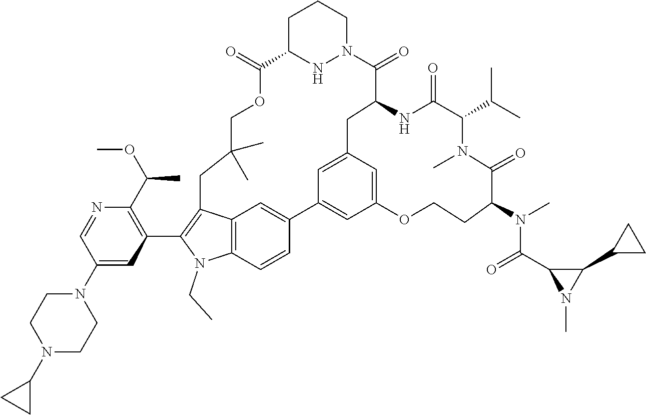 | B4 |
| 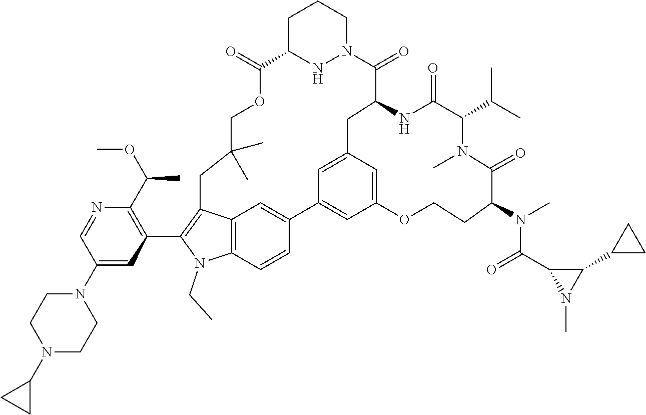 | B5 |
| B6 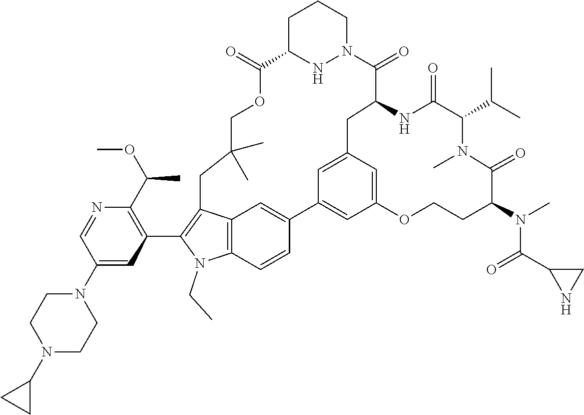 | B6 |
| 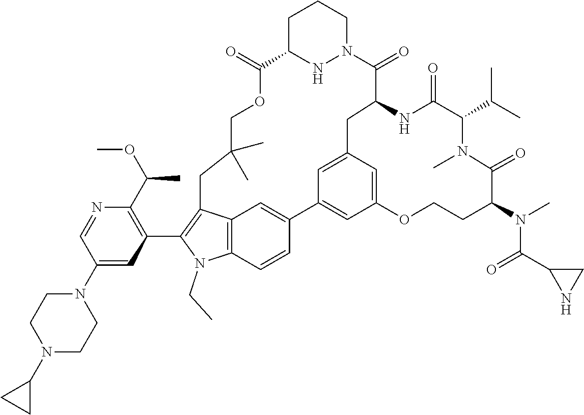 | B7 |
| 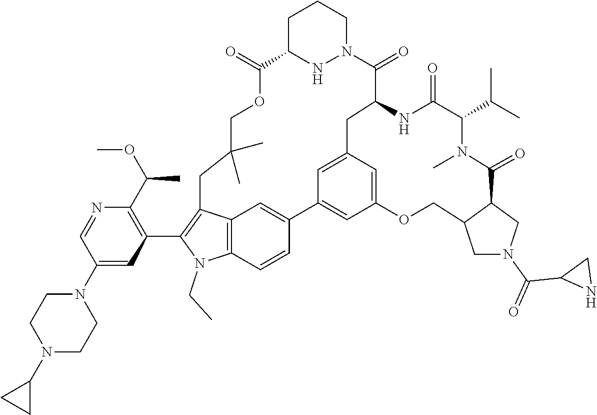 | B8 |
| 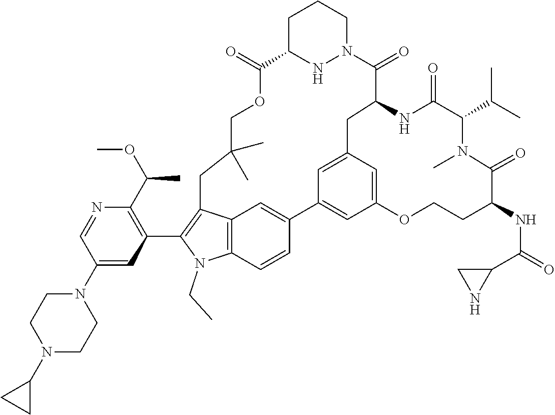 | B9 |
| 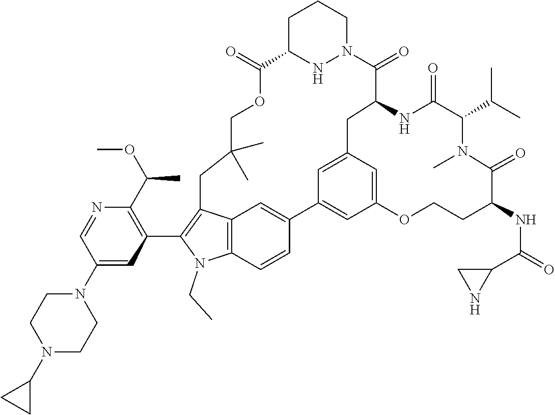 | B10 |
| 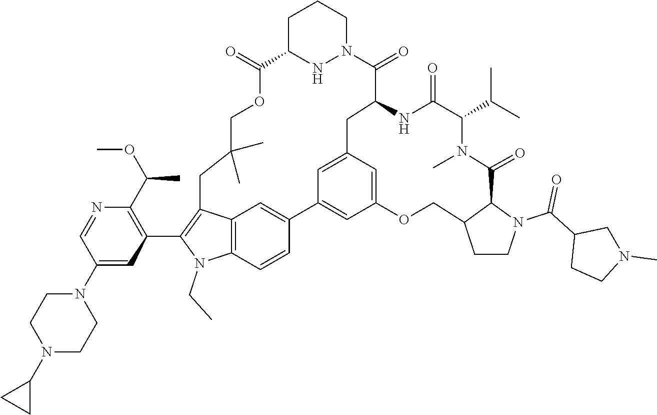 | B11 |
| 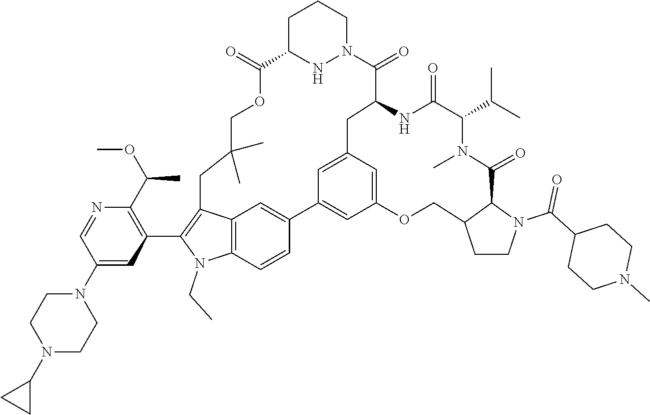 | B12 |
| 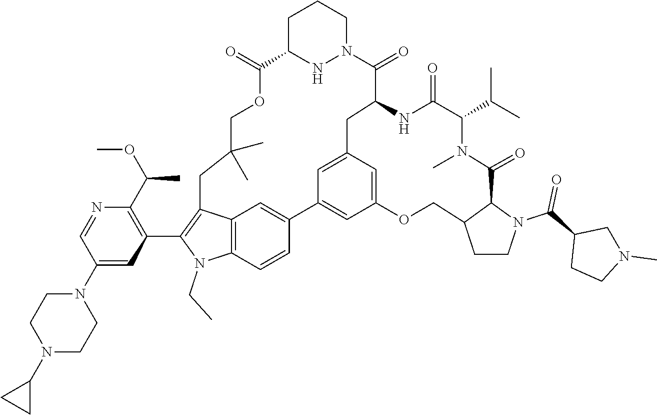 | B13 |
| 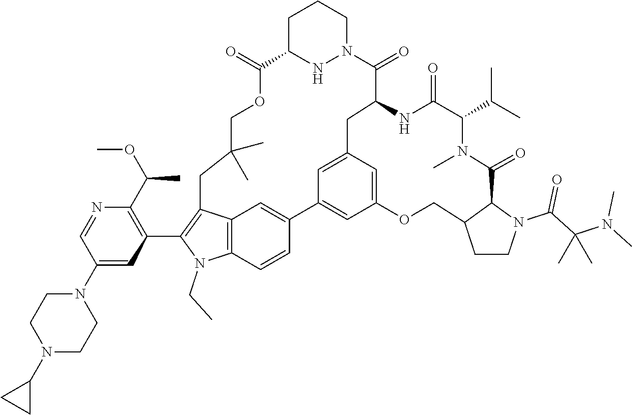 | B14 |
| 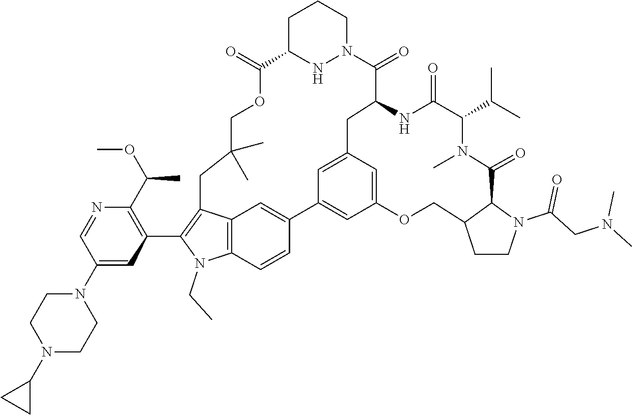 | B15 |
| 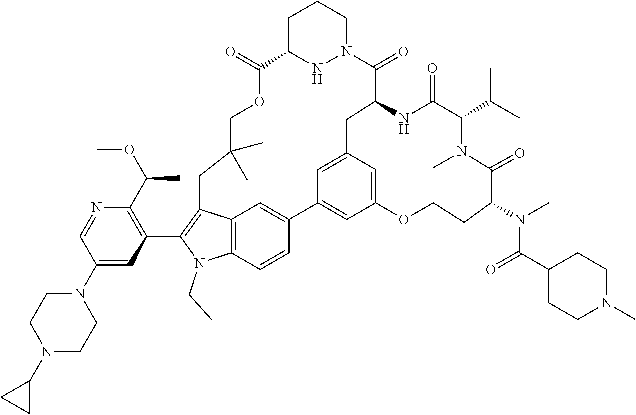 | B16 |
| 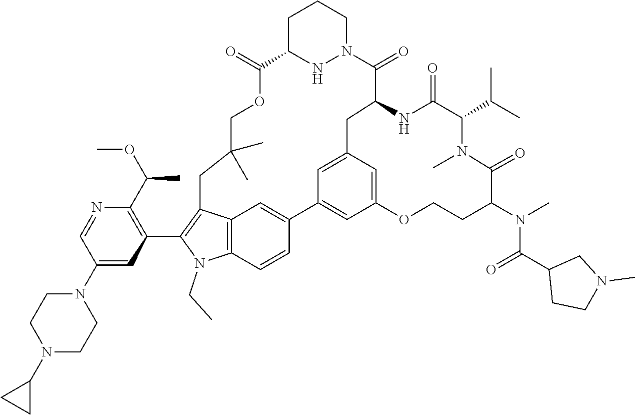 | B17 |
| 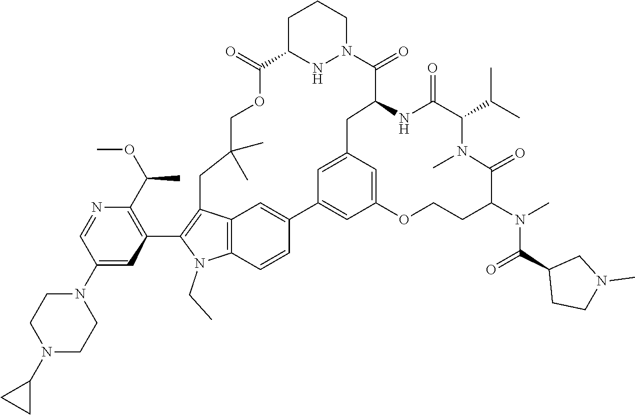 | B18 |
| 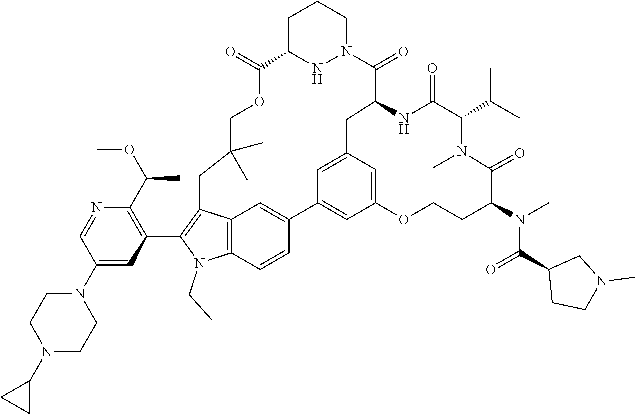 | B19 |
| 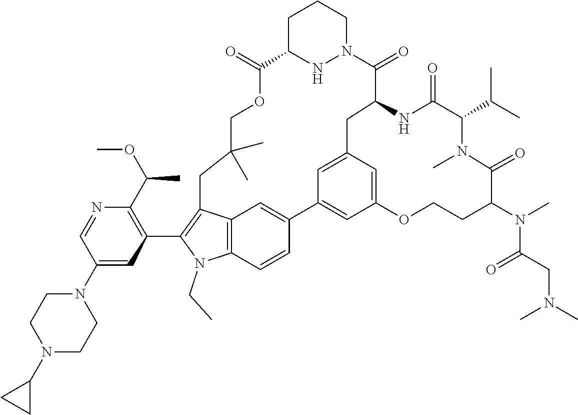 | B20 |
| 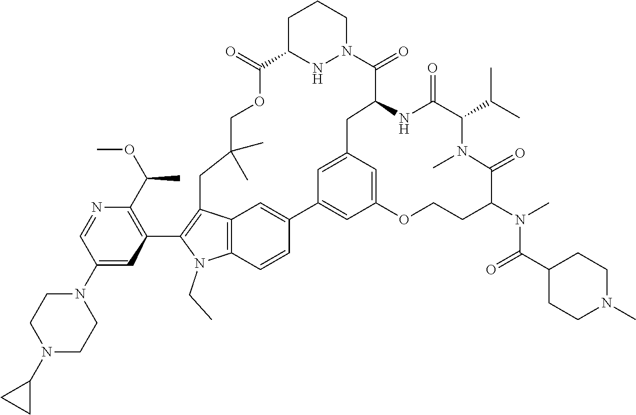 | B21 |
| 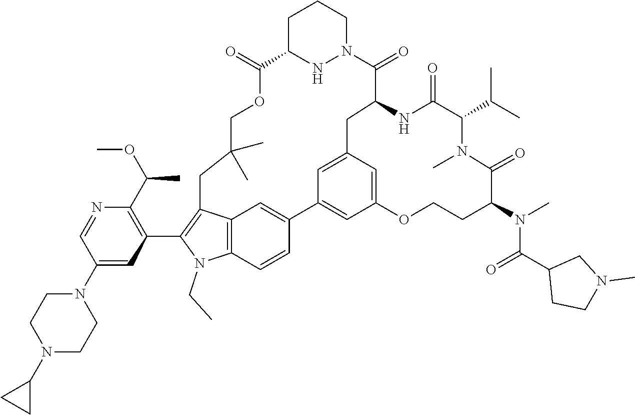 | B22 |
| 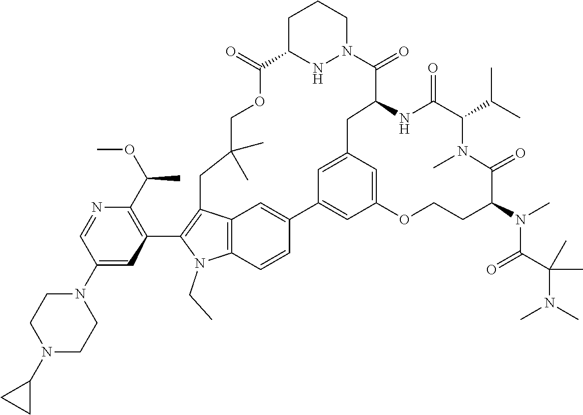 | B23 |
| 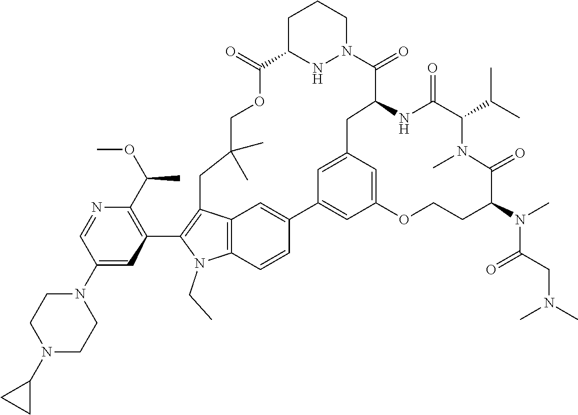 | B24 |
| 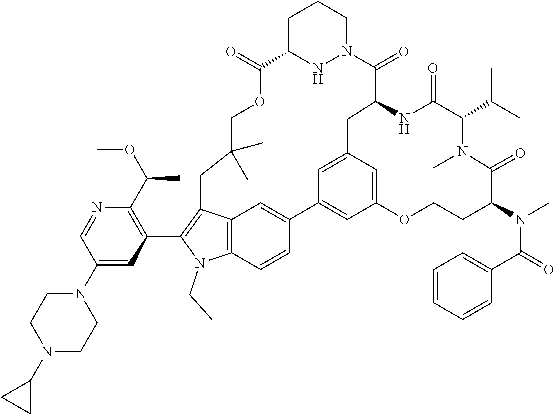 | B25 |
| 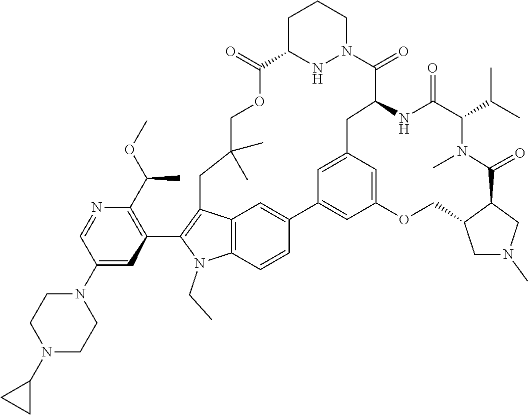 | B26 |
| 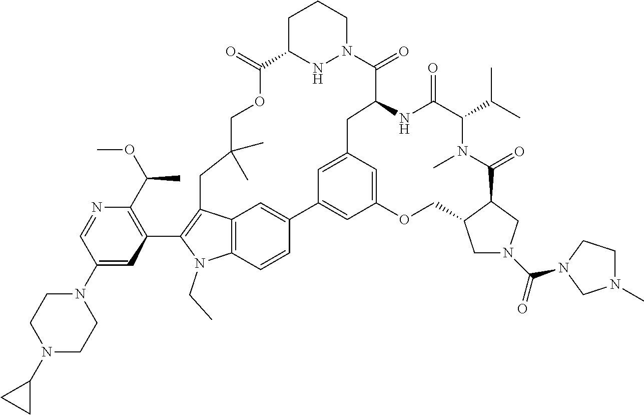 | B27 |
| 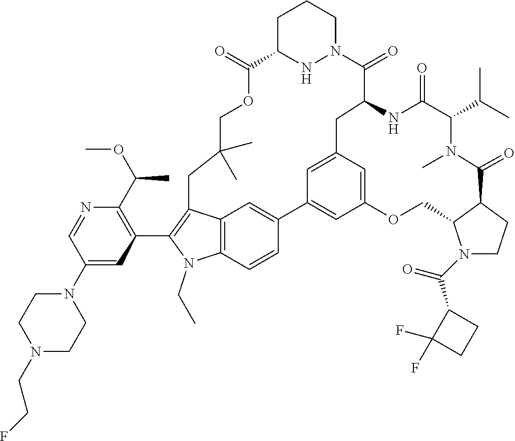 | B28 |
| 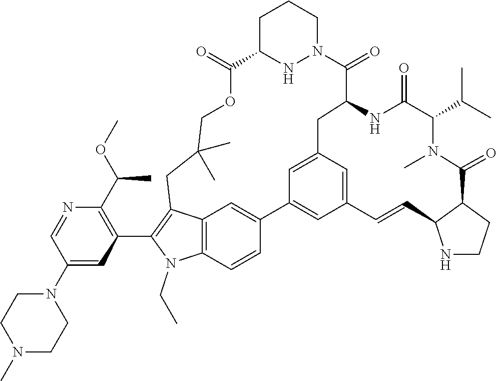 | B29 |
| 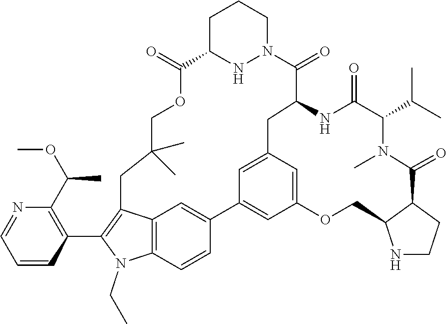 | B30 |
| 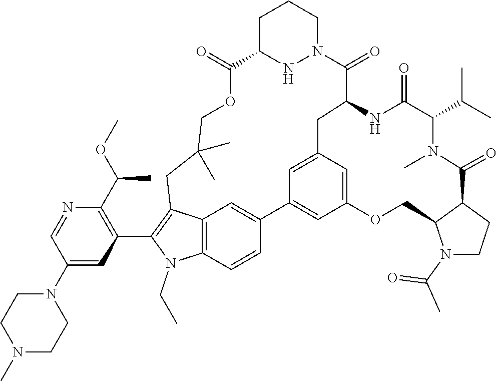 | B31 |
| 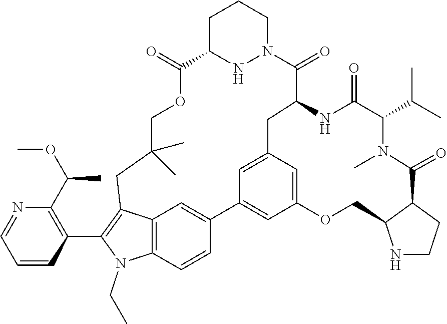 | B32 |
| 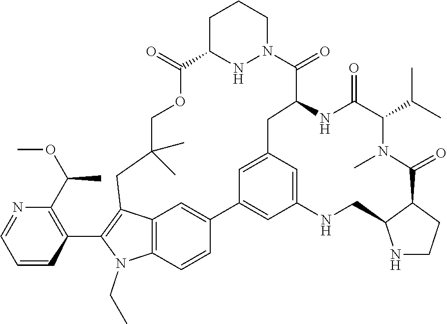 | B33 |

[symbol]

indicates data missing or illegible when filed

Also provided is a pharmaceutical composition comprising a compound of the present invention, or a pharmaceutically acceptable salt thereof, and a pharmaceutically acceptable excipient.

Compounds of the present invention are also adaptable for uses in antibody-drug conjugates as well as degrader applications.

Further provided is a method of treating cancer in a subject in need thereof, the method comprising administering to the subject a therapeutically effective amount of a compound of the present invention, or a pharmaceutically acceptable salt thereof. The cancer may, for example, be pancreatic cancer, colorectal cancer, non-small cell lung cancer, acute myeloid leukemia, multiple myeloma, thyroid gland adenocarcinoma, a myelodysplastic syndrome, or squamous cell lung carcinoma. In some embodiments, the cancer is pancreatic cancer, colorectal cancer, non-small cell lung cancer, acute myeloid leukemia, or multiple myeloma. In some embodiments, the cancer comprises a Ras mutation, such as K-Ras Q61H, H-Ras Q61H, or N-Ras Q61H. Other Ras mutations are described herein.

Further provided is a method of treating a Ras protein-related disorder in a subject in need thereof, the method comprising administering to the subject a therapeutically effective amount of a compound of the present invention, or a pharmaceutically acceptable salt thereof.

Further provided is a method of inhibiting a Ras protein in a cell, the method comprising contacting the cell with an effective amount of a compound of the present invention, or a pharmaceutically acceptable salt thereof. For example, the Ras protein is K-Ras Q61H, H-Ras Q61H, or N-Ras Q61H. Other Ras proteins are described herein. The cell may be a cancer cell, such as a pancreatic cancer cell, a colorectal cancer cell, a non-small cell lung cancer cell, an acute myeloid leukemia cell, a multiple myeloma cell, a thyroid gland adenocarcinoma cell, a myelodysplastic syndrome cell, or a squamous cell lung carcinoma cell. In some embodiments, the cell is a pancreatic cancer cell, a colorectal cancer cell, a non-small cell lung cancer cell, an acute myeloid leukemia cell, or a multiple myeloma cell, Other cancer types are described herein. The cell may be in vivo or in vitro.

With respect to compounds of the present invention, one stereoisomer may exhibit better inhibition than another stereoisomer. For example, one atropisomer may exhibit inhibition, whereas the other atropisomer may exhibit little or no inhibition.

In some embodiments, a method or use described herein further comprises administering an additional anti-cancer therapy. In some embodiments, the additional anti-cancer therapy is a HER2 inhibitor, an EGFR inhibitor, a second Ras inhibitor, a SHP2 inhibitor, an SOS1 inhibitor, a Raf inhibitor, a MEK inhibitor, an ERK inhibitor, a PI3K inhibitor, a PTEN inhibitor, an AKT inhibitor, an mTORC1 inhibitor, a BRAF inhibitor, a PD-L1 inhibitor, a PD-1 inhibitor, a CDK4/6 inhibitor, or a combination thereof. In some embodiments, the additional anticancer therapy is a SHP2 inhibitor. Other additional anti-cancer therapies are described herein.

### Methods of Synthesis

The compounds described herein may be made from commercially available starting materials or synthesized using known organic, inorganic, or enzymatic processes.

The compounds of the present invention can be prepared in a number of ways well known to those skilled in the art of organic synthesis. By way of example, compounds of the present invention can be synthesized using the methods described in the Schemes below, together with synthetic methods known in the art of synthetic organic chemistry, or variations thereon as appreciated by those skilled in the art. These methods include but are not limited to those methods described in the Schemes below.

Compounds of Tables 1 and 2 herein were prepared using methods disclosed herein or were prepared using methods disclosed herein combined with the knowledge of one of skill in the art.

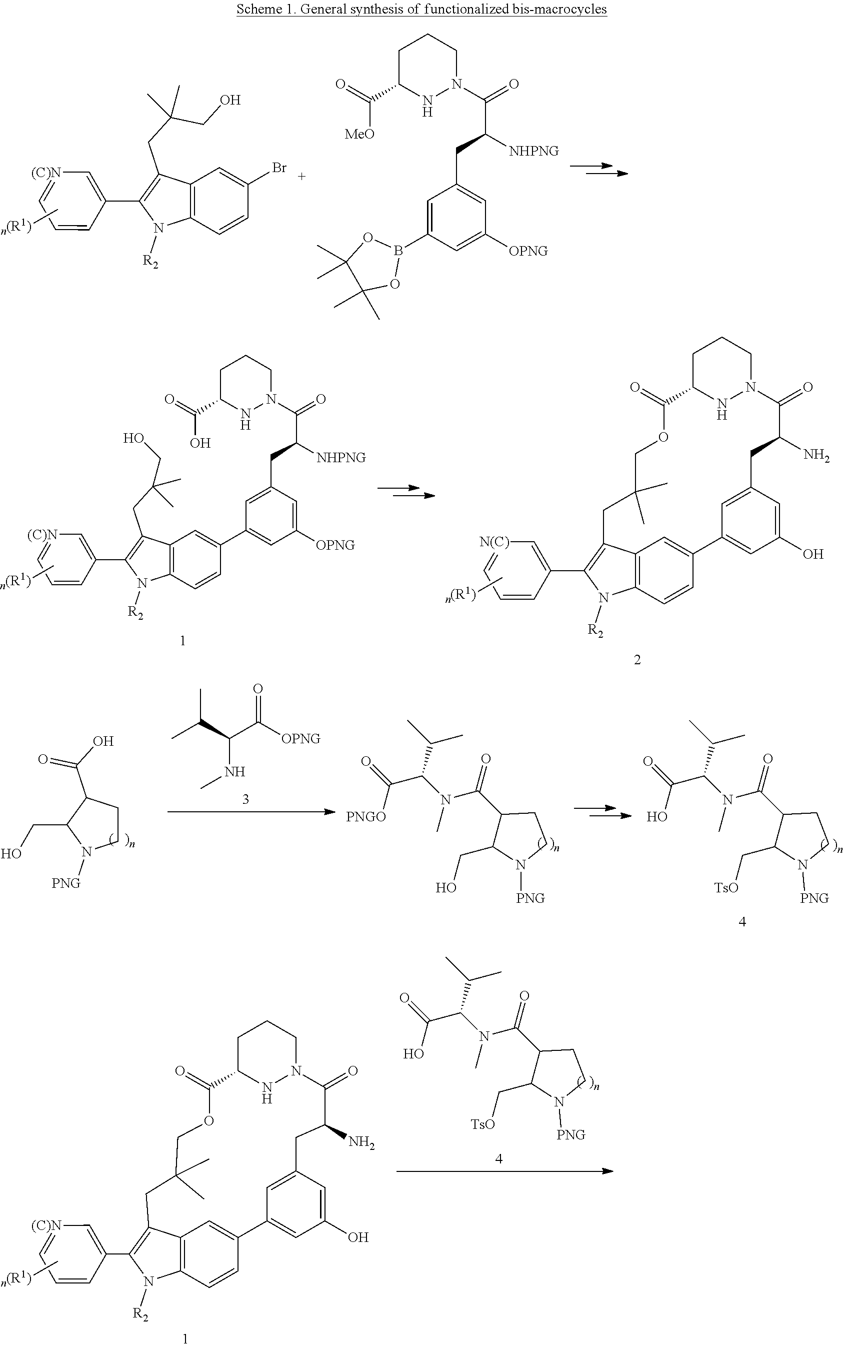

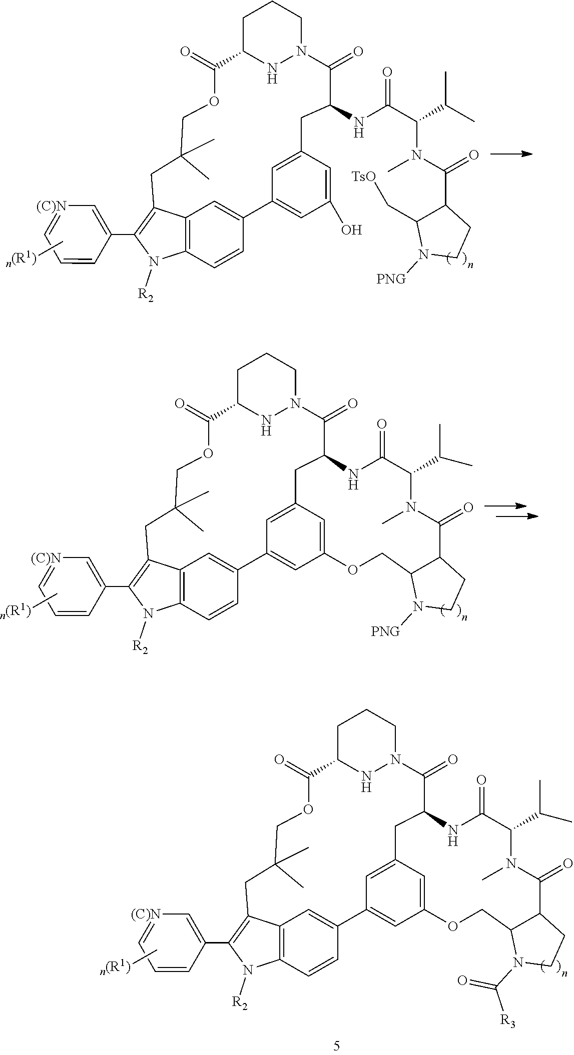

A general synthesis of functionalized bis-macrocycles is outlined in Scheme 1. An appropriately substituted biaryl intermediate (1) can be prepared in one step from an appropriately substituted 3-(5-bromo-2-iodo-1H-indol-3-yl)-2,2-dimethylpropan-1-ol intermediate and an appropriately substituted methyl piperazic ester-containing aryl boronic ester by a palladium mediated coupling followed by ester hydrolysis. Macrolactonization followed by amine and phenol deprotection can yield macrocyclic ester (2).

An appropriately substituted 2-(tosyloxymethyl)-3-(amido) cyclic amine (3) can be prepared by the coupling of an O-protected N-methyl-L-valine (4) with an appropriately substituted 2-(hydroxymethyl)-3-carboxylate cyclic amine using a peptide coupling reagent, followed by tosylation of the alcohol and carboxylic acid deprotection.

The final functionalized bis-macrocycles can then be made by the peptide coupling of macrocyclic ester (1) with intermediate (3) followed by macrocyclic ether formation in the presence of base. Deprotection and coupling of the amine with an appropriately substituted carboxylic acid (or other coupling partner) results in a bis-macrocyclic product (5).

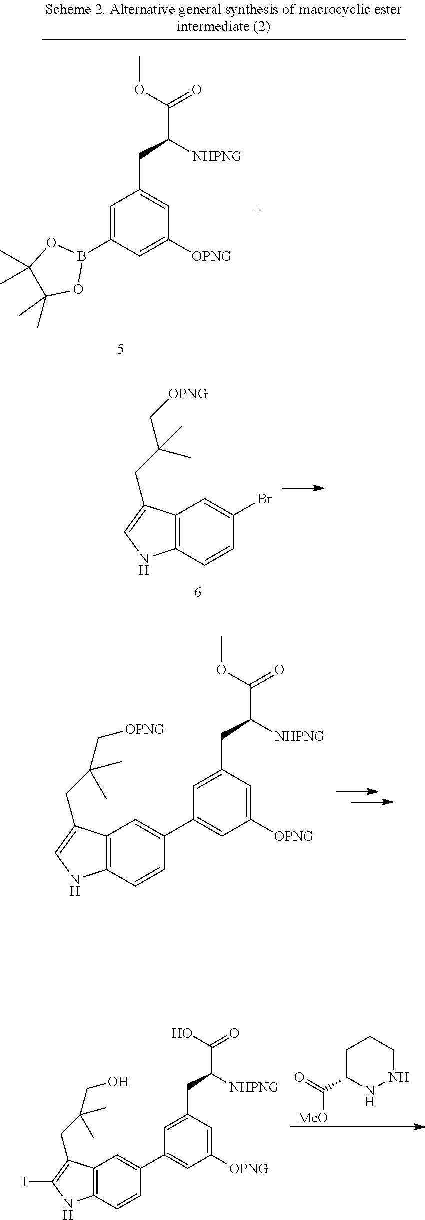

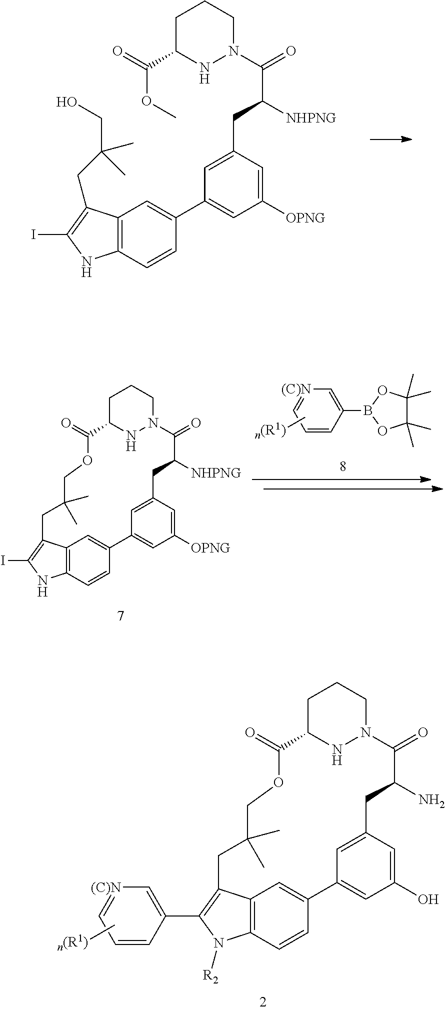

Alternatively, macrocyclic ester intermediate (2) can be prepared as described in Scheme 2. An appropriately substituted aryl boronic ester (5) and be coupled with an appropriately protected 3-(5-bromo-indol-3-yl)-2,2-dimethylpropan-1-ol (6) in the presence of a palladium catalyst. This can be followed by indole iodination, alcohol deprotection, and ester hydrolysis. Subsequent coupling with methyl (S)-piperazic ester, ester hydrolysis and macrolactonization can result in iodinated macrocyclic intermediate (7). Coupling in the presence of palladium catalyst with an appropriately substituted aryl boronic ester (8) and indole N-alkylation, followed by subsequent amine and phenol deprotection result in intermediate (2).

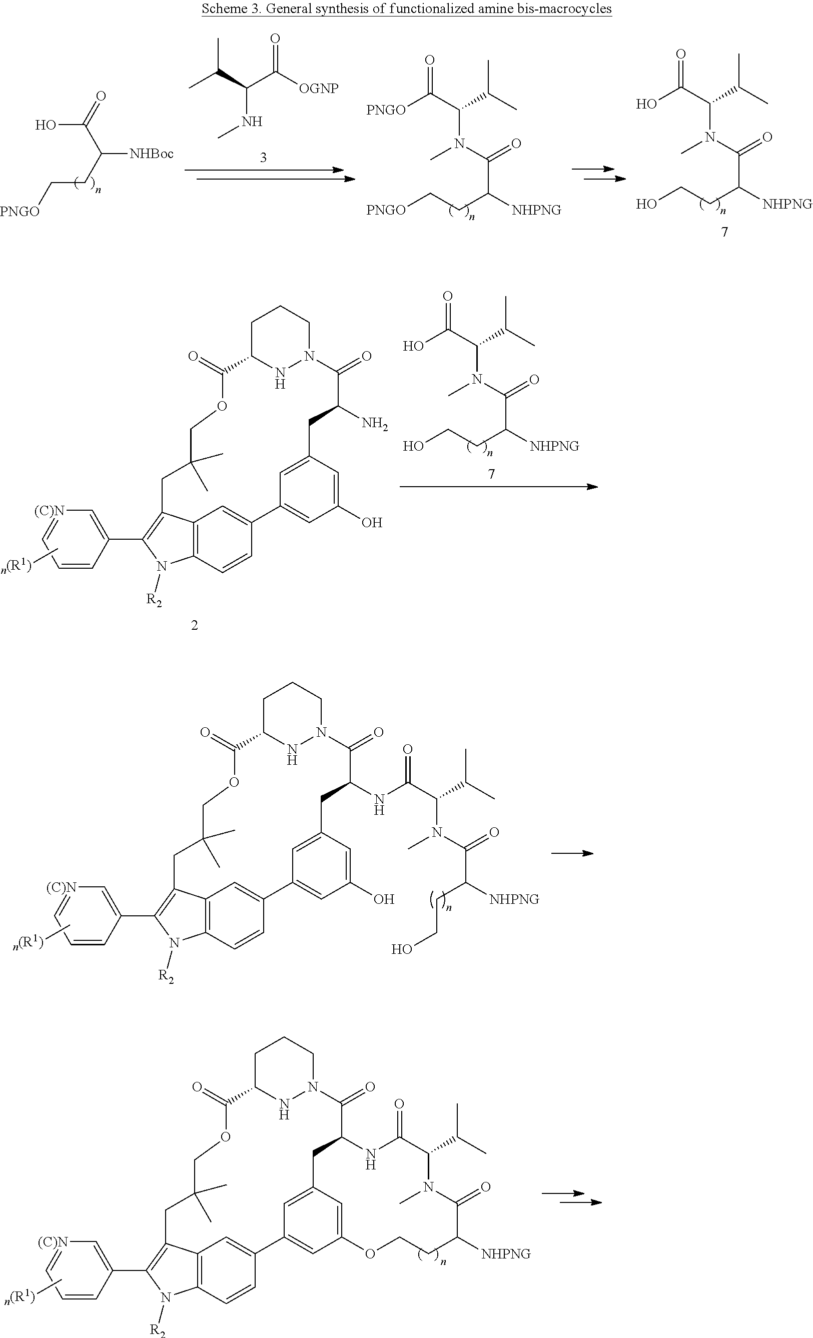

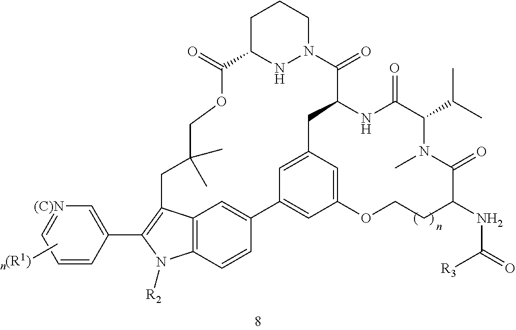

A general synthesis of functionalized bis-macrocycles is outlined in Scheme 3. An appropriately protected hydroxyalkyl amino acid can be coupled with O-protected N-methyl-L-valine (3) by a peptide coupling reagent. Subsequent alcohol and carboxylic acid deprotection can produce appropriately substituted intermediate (7).

A protected amine bis-macrocyclic intermediate can be made by peptide coupling of macrocyclic ester intermediate (2) with carboxylic acid (7) followed by bis-macrocyclic ether formation in the presence of triphenylphosphine and an azodicarboxylate. Deprotection and coupling of the amine with an appropriately substituted carboxylic acid (or other coupling partner) results in final bis-macrocyclic product (8).

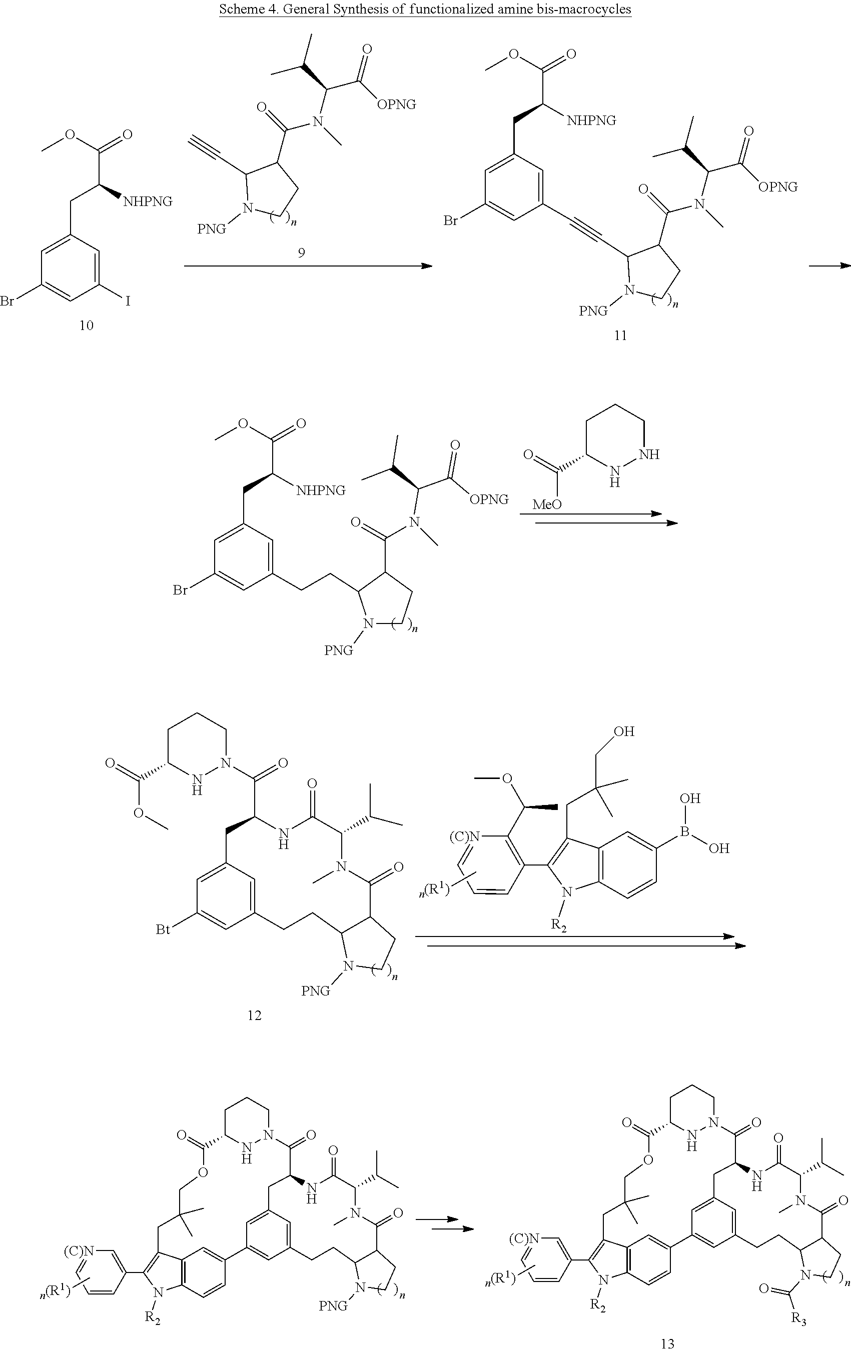

A general synthesis of functionalized bis-macrocycles is outlined in Scheme 4. An appropriately substituted terminal alkyne (9) can be coupled with an appropriately substituted iodinated bromoarene (10) in the presence of a palladium catalyst. Subsequent reduction of aryl alkyne intermediate (11) followed by amino acid N-deprotection, carboxylic acid deprotection, macrocyclization in the presence of a peptide coupling reagent, ester hydrolysis, and peptide coupling with methyl (S)-piperazic ester can yield macrocyclic intermediate (12). Functionalized bis-macrocycle (13) can then be obtained through a palladium mediated coupling with an appropriately substituted 3-(5-boronate-indol-3-yl)-2,2-dimethylpropan-1-ol, macrolactonization, amine deprotection, and coupling of the amine with an appropriately substituted carboxylic acid (or other coupling partner).

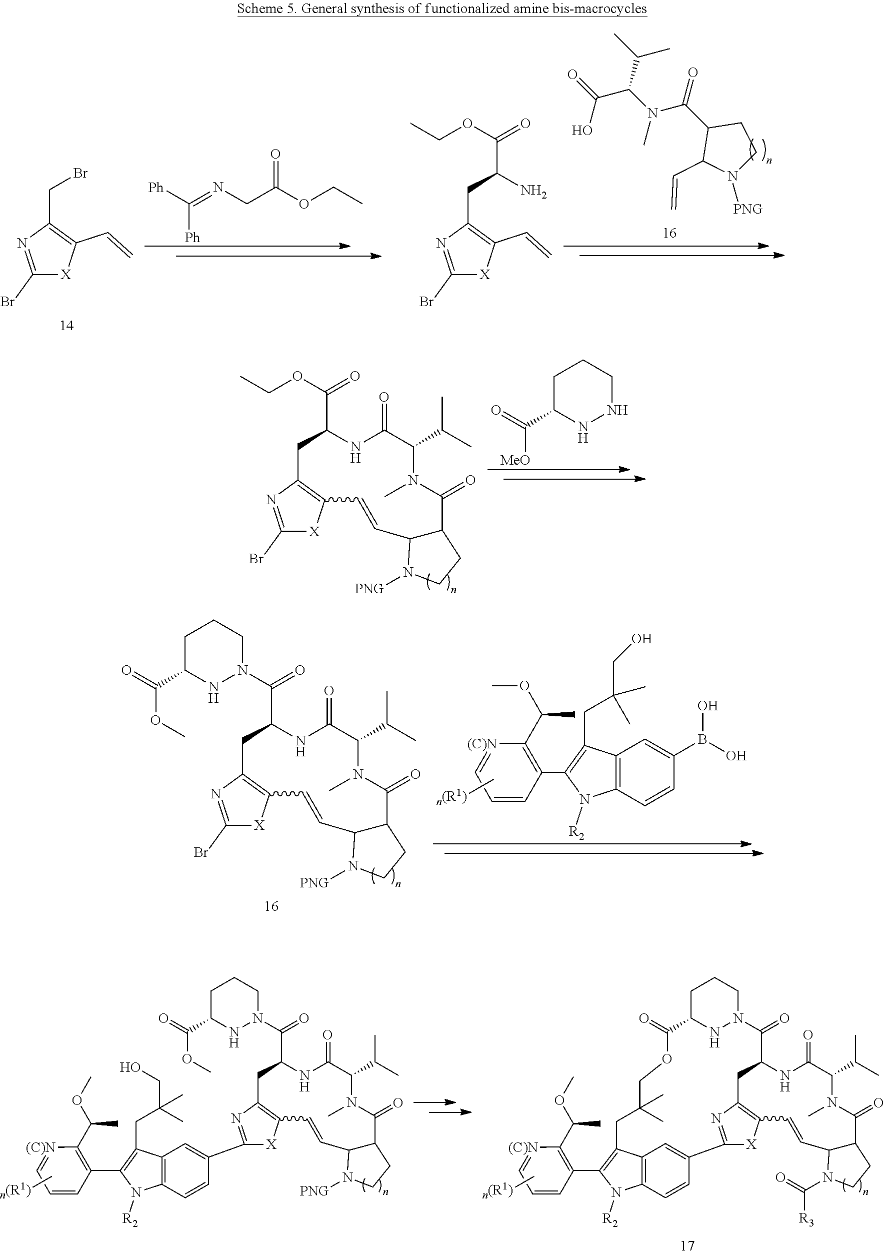

A general synthesis of functionalized bis-macrocycles is outlined in Scheme 5. An appropriately substituted 2-bromo-4-bromomethyl-5-ethenyl 5-membered heteroarene (14) can react with ethyl 2-((diphenylethylene)amino) acetate in the presence of base and a chiral auxiliary. Subsequent amide coupling with an appropriately substituted 2-(ethenyl)-3-(amido) cyclic amine (15) followed by an olefin metathesis reaction, ester hydrolysis, and an amide coupling reaction with methyl (S)-piperazic ester can yield macrocycle (16).

Functionalized amine bis-macrocycle (17) can then be obtained through a palladium mediated coupling with an appropriately substituted 3-(5-boronate-indol-3-yl)-2,2-dimethylpropan-1-ol, methyl ester hydrolysis, macrolactonization, amine deprotection, and coupling of the amine with an appropriately substituted carboxylic acid (or other coupling partner).

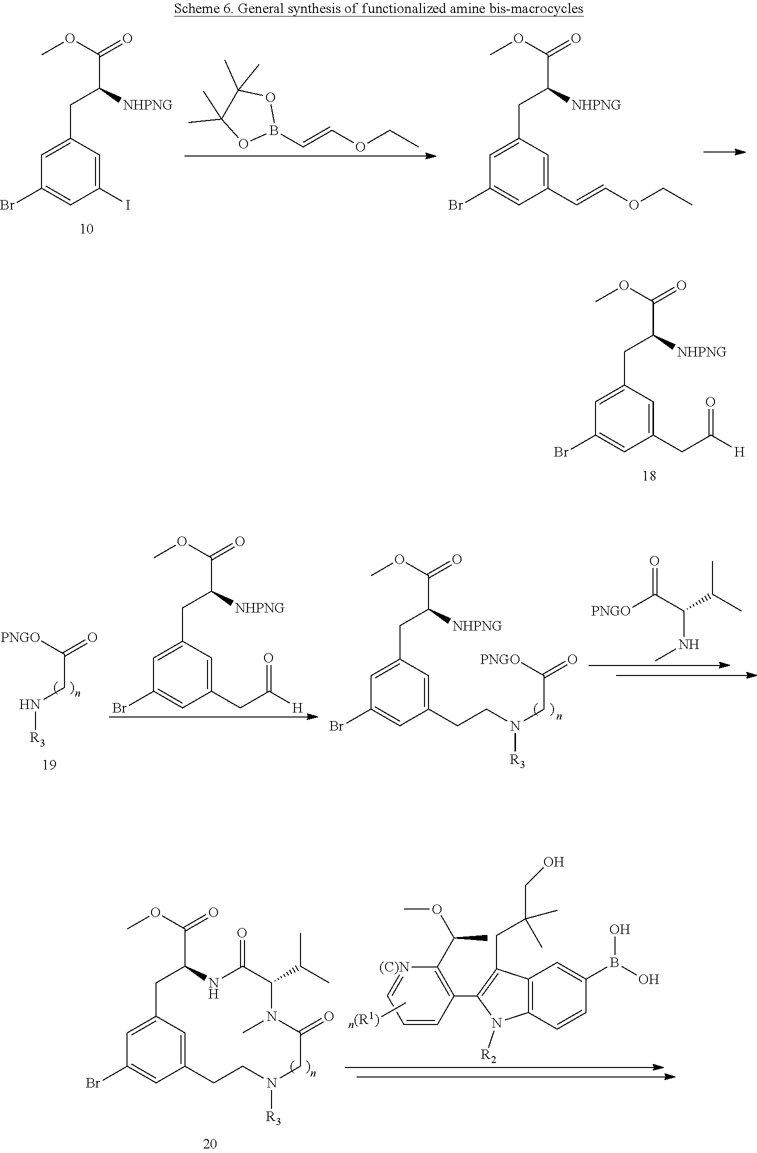

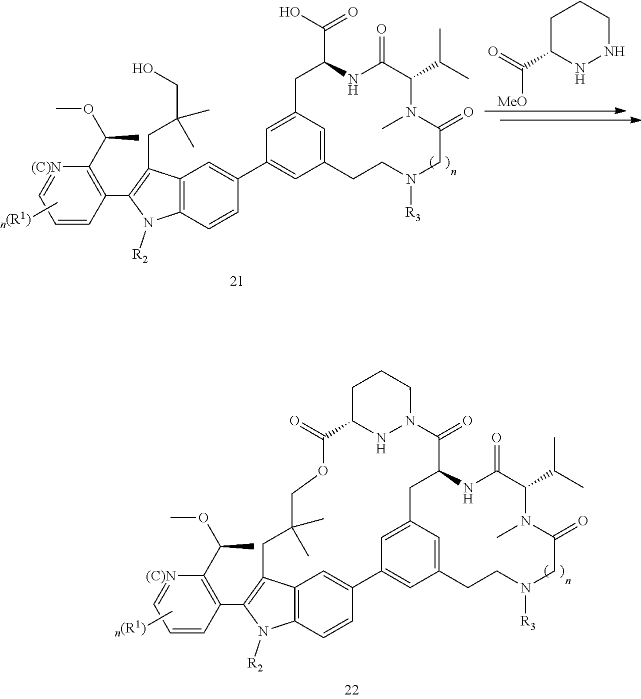

A general synthesis of functionalized bis-macrocycles is outlined in Scheme 6. An appropriately substituted iodinated bromoarene (10) can be coupled with a vinyl boronate ester in the presence of a palladium catalyst. Hydrolysis of the vinyl ether in the presence of acid can yield aldehyde (18).

An appropriately N-functionalized O-protected amino acid (19) can be coupled with an aldehyde (18) in the presence of acid and a reducing agent. This can be followed by carboxylic acid deprotection and coupling of an O-protected N-methyl-L-valine (3) in the presence of an amide coupling reagent. Subsequent carboxylate and amine deprotections followed by cyclization in the presence of a peptide coupling reagent hydrolysis can yield macrocyclic intermediate (20).

An appropriately substituted biaryl intermediate (21) can then be prepared in two steps through coupling of intermediate (20) with an appropriately substituted 3-(5-boronate-indol-3-yl)-2,2-dimethylpropan-1-ol by a palladium mediated coupling followed by ester hydrolysis. Subsequent coupling with methyl (S)-piperazic ester by a peptide coupling reagent, ester hydrolysis, and macrolactonization can yield functionalized amine bis-macrocycle (22).

In any embodiment herein, a compound of the present invention may be modified with a substituent as found in any one or more of the following applications using methodologies described in these applications in combination with methods provided herein and know to those of skill in the art: WO 2024/060966, WO 2024/017859, WO 2024/008834, WO 2024/008610, WO 2023/232776, WO 2023/208005, WO 2023/086341, WO 2023/025832, WO 2023/015559, CN 117720556, CN 117720555, CN 117720554, CN 117534687, CN 117534685 and CN 117534684, each incorporated herein by reference in their entireties.

### Pharmaceutical Compositions and Methods of Use

The compounds with which the invention is concerned are Ras inhibitors and are useful in the treatment of cancer. Accordingly, one embodiment of the present invention provides pharmaceutical compositions containing a compound of the invention or a pharmaceutically acceptable salt thereof, and a pharmaceutically acceptable excipient, as well as methods of using the compounds of the invention to prepare such compositions.

As used herein, the term “pharmaceutical composition” refers to a compound, such as a compound of the present invention, or a pharmaceutically acceptable salt thereof, formulated together with a pharmaceutically acceptable excipient.

In some embodiments, a compound is present in a pharmaceutical composition in unit dose amount appropriate for administration in a therapeutic regimen that shows a statistically significant probability of achieving a predetermined therapeutic effect when administered to a relevant population. In some embodiments, pharmaceutical compositions may be specially formulated for administration in solid or liquid form, including those adapted for the following: oral administration, for example, drenches (aqueous or non-aqueous solutions or suspensions), tablets, e.g., those targeted for buccal, sublingual, and systemic absorption, boluses, powders, granules, pastes for application to the tongue; parenteral administration, for example, by subcutaneous, intramuscular, intravenous or epidural injection as, for example, a sterile solution or suspension, or sustained-release formulation; topical application, for example, as a cream, ointment, or a controlled-release patch or spray applied to the skin, lungs, or oral cavity; intravaginally or intrarectally, for example, as a pessary, cream, or foam; sublingually; ocularly; transdermally; or nasally, pulmonary, and to other mucosal surfaces.

A “pharmaceutically acceptable excipient,” as used herein, refers to any inactive ingredient (for example, a vehicle capable of suspending or dissolving the active compound) having the properties of being nontoxic and non-inflammatory in a subject. Typical excipients include, for example: antiadherents, antioxidants, binders, coatings, compression aids, disintegrants, dyes (colors), emollients, emulsifiers, fillers (diluents), film formers or coatings, flavors, fragrances, glidants (flow enhancers), lubricants, preservatives, printing inks, sorbents, suspending or dispersing agents, sweeteners, or waters of hydration. Excipients include, but are not limited to: butylated optionally substituted hydroxyltoluene (BHT), calcium carbonate, calcium phosphate (dibasic), calcium stearate, croscarmellose, crosslinked polyvinyl pyrrolidone, citric acid, crospovidone, cysteine, ethylcellulose, gelatin, optionally substituted hydroxylpropyl cellulose, optionally substituted hydroxylpropyl methylcellulose, lactose, magnesium stearate, maltitol, mannitol, methionine, methylcellulose, methyl paraben, microcrystalline cellulose, polyethylene glycol, polyvinyl pyrrolidone, povidone, pregelatinized starch, propyl paraben, retinyl palmitate, shellac, silicon dioxide, sodium carboxymethyl cellulose, sodium citrate, sodium starch glycolate, sorbitol, starch (corn), stearic acid, stearic acid, sucrose, talc, titanium dioxide, vitamin A, vitamin E, vitamin C, and xylitol. Those of ordinary skill in the art are familiar with a variety of agents and materials useful as excipients. See, e.g., e.g., Ansel, et al., Ansel's Pharmaceutical Dosage Forms and Drug Delivery Systems. Philadelphia: Lippincott, Williams & Wilkins, 2004; Gennaro, et al., Remington: The Science and Practice of Pharmacy. Philadelphia: Lippincott, Williams & Wilkins, 2000; and Rowe, Handbook of Pharmaceutical Excipients. Chicago, Pharmaceutical Press, 2005. In some embodiments, a composition includes at least two different pharmaceutically acceptable excipients.

Compounds described herein, whether expressly stated or not, may be provided or utilized in salt form, e.g., a pharmaceutically acceptable salt form, unless expressly stated to the contrary. The term “pharmaceutically acceptable salt,” as use herein, refers to those salts of the compounds described herein that are, within the scope of sound medical judgment, suitable for use in contact with the tissues of humans and other animals without undue toxicity, irritation, allergic response and the like, and are commensurate with a reasonable benefit/risk ratio. Pharmaceutically acceptable salts are well known in the art. For example, pharmaceutically acceptable salts are described in: Berge et al., J. Pharmaceutical Sciences 66:1-19, 1977 and in Pharmaceutical Salts: Properties, Selection, and Use, (Eds. P. H. Stahl and C.G. Wermuth), Wiley-VCH, 2008. The salts can be prepared in situ during the final isolation and purification of the compounds described herein or separately by reacting the free base group with a suitable organic acid.

The compounds of the invention may have ionizable groups so as to be capable of preparation as pharmaceutically acceptable salts. These salts may be acid addition salts involving inorganic or organic acids or the salts may, in the case of acidic forms of the compounds of the invention, be prepared from inorganic or organic bases. In some embodiments, the compounds are prepared or used as pharmaceutically acceptable salts prepared as addition products of pharmaceutically acceptable acids or bases. Suitable pharmaceutically acceptable acids and bases are well-known in the art, such as hydrochloric, sulfuric, hydrobromic, acetic, lactic, citric, or tartaric acids for forming acid addition salts, and potassium hydroxide, sodium hydroxide, ammonium hydroxide, caffeine, various amines, and the like for forming basic salts. Methods for preparation of the appropriate salts are well-established in the art.

Representative acid addition salts include acetate, adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, fumarate, glucoheptonate, glycerophosphate, hemisulfate, heptonate, hexanoate, hydrobromide, hydrochloride, hydroiodide, 2-optionally substituted hydroxyl-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, toluenesulfonate, undecanoate, valerate salts and the like. Representative alkali or alkaline earth metal salts include sodium, lithium, potassium, calcium, magnesium, and the like, as well as nontoxic ammonium, quaternary ammonium, and amine cations, including, but not limited to ammonium, tetramethylammonium, tetraethylammonium, methylamine, dimethylamine, trimethylamine, triethylamine, ethylamine, and the like.

As used herein, the term “subject” refers to any member of the animal kingdom. In some embodiments, “subject” refers to humans, at any stage of development. In some embodiments, “subject” refers to a human patient. In some embodiments, “subject” refers to non-human animals. In some embodiments, the non-human animal is a mammal (e.g., a rodent, a mouse, a rat, a rabbit, a monkey, a dog, a cat, a sheep, cattle, a primate, or a pig). In some embodiments, subjects include, but are not limited to, mammals, birds, reptiles, amphibians, fish, or worms. In some embodiments, a subject may be a transgenic animal, genetically-engineered animal, or a clone.

As used herein, the term “dosage form” refers to a physically discrete unit of a compound (e.g., a compound of the present invention) for administration to a subject. Each unit contains a predetermined quantity of compound. In some embodiments, such quantity is a unit dosage amount (or a whole fraction thereof) appropriate for administration in accordance with a dosing regimen that has been determined to correlate with a desired or beneficial outcome when administered to a relevant population (i.e., with a therapeutic dosing regimen). Those of ordinary skill in the art appreciate that the total amount of a therapeutic composition or compound administered to a particular subject is determined by one or more attending physicians and may involve administration of multiple dosage forms.

As used herein, the term “dosing regimen” refers to a set of unit doses (typically more than one) that are administered individually to a subject, typically separated by periods of time. In some embodiments, a given therapeutic compound (e.g., a compound of the present invention) has a recommended dosing regimen, which may involve one or more doses. In some embodiments, a dosing regimen comprises a plurality of doses each of which are separated from one another by a time period of the same length; in some embodiments, a dosing regimen comprises a plurality of doses and at least two different time periods separating individual doses. In some embodiments, all doses within a dosing regimen are of the same unit dose amount. In some embodiments, different doses within a dosing regimen are of different amounts. In some embodiments, a dosing regimen comprises a first dose in a first dose amount, followed by one or more additional doses in a second dose amount different from the first dose amount. In some embodiments, a dosing regimen comprises a first dose in a first dose amount, followed by one or more additional doses in a second dose amount same as the first dose amount. In some embodiments, a dosing regimen is correlated with a desired or beneficial outcome when administered across a relevant population (i.e., is a therapeutic dosing regimen).

A “therapeutic regimen” refers to a dosing regimen whose administration across a relevant population is correlated with a desired or beneficial therapeutic outcome.

The term “treatment” (also “treat” or “treating”), in its broadest sense, refers to any administration of a substance (e.g., a compound of the present invention) that partially or completely alleviates, ameliorates, relieves, inhibits, delays onset of, reduces severity of, or reduces incidence of one or more symptoms, features, or causes of a particular disease, disorder, or condition. In some embodiments, such treatment may be administered to a subject who does not exhibit signs of the relevant disease, disorder, or condition or of a subject who exhibits only early signs of the disease, disorder, or condition. Alternatively, or additionally, in some embodiments, treatment may be administered to a subject who exhibits one or more established signs of the relevant disease, disorder, or condition. In some embodiments, treatment may be of a subject who has been diagnosed as suffering from the relevant disease, disorder, or condition. In some embodiments, treatment may be of a subject known to have one or more susceptibility factors that are statistically correlated with increased risk of development of the relevant disease, disorder, or condition.

The term “therapeutically effective amount” means an amount that is sufficient, when administered to a population suffering from or susceptible to a disease, disorder, or condition in accordance with a therapeutic dosing regimen, to treat the disease, disorder, or condition. In some embodiments, a therapeutically effective amount is one that reduces the incidence or severity of, or delays onset of, one or more symptoms of the disease, disorder, or condition. Those of ordinary skill in the art will appreciate that the term “therapeutically effective amount” does not in fact require successful treatment be achieved in a particular individual. Rather, a therapeutically effective amount may be that amount that provides a particular desired pharmacological response in a significant number of subjects when administered to patients in need of such treatment. It is specifically understood that particular subjects may, in fact, be “refractory” to a “therapeutically effective amount.” In some embodiments, reference to a therapeutically effective amount may be a reference to an amount as measured in one or more specific tissues (e.g., a tissue affected by the disease, disorder, or condition) or fluids (e.g., blood, saliva, serum, sweat, tears, urine). Those of ordinary skill in the art will appreciate that, in some embodiments, a therapeutically effective amount may be formulated or administered in a single dose. In some embodiments, a therapeutically effective amount may be formulated or administered in a plurality of doses, for example, as part of a dosing regimen.

For use as treatment of subjects, the compounds of the invention, or a pharmaceutically acceptable salt thereof, can be formulated as pharmaceutical or veterinary compositions. Depending on the subject to be treated, the mode of administration, and the type of treatment desired, e.g., prevention, prophylaxis, or therapy, the compounds, or a pharmaceutically acceptable salt thereof, are formulated in ways consonant with these parameters. A summary of such techniques may be found in Remington: The Science and Practice of Pharmacy, 21^{st} Edition, Lippincott Williams & Wilkins, (2005); and Encyclopedia of Pharmaceutical Technology, eds. J. Swarbrick and J. C. Boylan, 1988-1999, Marcel Dekker, New York, each of which is incorporated herein by reference.

Compositions can be prepared according to conventional mixing, granulating, or coating methods, respectively, and the present pharmaceutical compositions can contain from about 0.1% to about 99%, from about 5% to about 90%, or from about 1% to about 20% of a compound of the present invention, or pharmaceutically acceptable salt thereof, by weight or volume. In some embodiments, compounds, or a pharmaceutically acceptable salt thereof, described herein may be present in amounts totaling 1-95% by weight of the total weight of a composition, such as a pharmaceutical composition.

The composition may be provided in a dosage form that is suitable for intraarticular, oral, parenteral (e.g., intravenous, intramuscular), rectal, cutaneous, subcutaneous, topical, transdermal, sublingual, nasal, vaginal, intravesicular, intraurethral, intrathecal, epidural, aural, or ocular administration, or by injection, inhalation, or direct contact with the nasal, genitourinary, reproductive, or oral mucosa. Thus, the pharmaceutical composition may be in the form of, e.g., tablets, capsules, pills, powders, granulates, suspensions, emulsions, solutions, gels including hydrogels, pastes, ointments, creams, plasters, drenches, osmotic delivery devices, suppositories, enemas, injectables, implants, sprays, preparations suitable for iontophoretic delivery, or aerosols. The compositions may be formulated according to conventional pharmaceutical practice.

As used herein, the term “administration” refers to the administration of a composition (e.g., a compound, or a preparation that includes a compound as described herein) to a subject or system. Administration to an animal subject (e.g., to a human) may be by any appropriate route. For example, in some embodiments, administration may be bronchial (including by bronchial instillation), buccal, enteral, interdermal, intra-arterial, intradermal, intragastric, intramedullary, intramuscular, intranasal, intraperitoneal, intrathecal, intravenous, intraventricular, mucosal, nasal, oral, rectal, subcutaneous, sublingual, topical, tracheal (including by intratracheal instillation), transdermal, vaginal, or vitreal.

Formulations may be prepared in a manner suitable for systemic administration or topical or local administration. Systemic formulations include those designed for injection (e.g., intramuscular, intravenous, or subcutaneous injection) or may be prepared for transdermal, transmucosal, or oral administration. A formulation will generally include a diluent as well as, in some cases, adjuvants, buffers, preservatives and the like. Compounds, or a pharmaceutically acceptable salt thereof, can be administered also in liposomal compositions or as microemulsions.

For injection, formulations can be prepared in conventional forms as liquid solutions or suspensions or as solid forms suitable for solution or suspension in liquid prior to injection or as emulsions. Suitable excipients include, for example, water, saline, dextrose, glycerol, and the like. Such compositions may also contain amounts of nontoxic auxiliary substances such as wetting or emulsifying agents, pH buffering agents and the like, such as, for example, sodium acetate, sorbitan monolaurate, and so forth.

Various sustained release systems for drugs have also been devised. See, for example, U.S. Pat. No. 5,624,677.

Systemic administration may also include relatively noninvasive methods such as the use of suppositories, transdermal patches, transmucosal delivery and intranasal administration. Oral administration is also suitable for compounds of the invention, or a pharmaceutically acceptable salt thereof. Suitable forms include syrups, capsules, and tablets, as is understood in the art.

Each compound, or a pharmaceutically acceptable salt thereof, as described herein, may be formulated in a variety of ways that are known in the art. For example, the first and second agents of the combination therapy may be formulated together or separately. Other modalities of combination therapy are described herein.

The individually or separately formulated agents can be packaged together as a kit. Non-limiting examples include, but are not limited to, kits that contain, e.g., two pills, a pill and a powder, a suppository and a liquid in a vial, two topical creams, etc. The kit can include optional components that aid in the administration of the unit dose to subjects, such as vials for reconstituting powder forms, syringes for injection, customized IV delivery systems, inhalers, etc. Additionally, the unit dose kit can contain instructions for preparation and administration of the compositions. The kit may be manufactured as a single use unit dose for one subject, multiple uses for a particular subject (at a constant dose or in which the individual compounds, or a pharmaceutically acceptable salt thereof, may vary in potency as therapy progresses); or the kit may contain multiple doses suitable for administration to multiple subjects (“bulk packaging”). The kit components may be assembled in cartons, blister packs, bottles, tubes, and the like.

Formulations for oral use include tablets containing the active ingredient(s) in a mixture with non-toxic pharmaceutically acceptable excipients. These excipients may be, for example, inert diluents or fillers (e.g., sucrose, sorbitol, sugar, mannitol, microcrystalline cellulose, starches including potato starch, calcium carbonate, sodium chloride, lactose, calcium phosphate, calcium sulfate, or sodium phosphate); granulating and disintegrating agents (e.g., cellulose derivatives including microcrystalline cellulose, starches including potato starch, croscarmellose sodium, alginates, or alginic acid); binding agents (e.g., sucrose, glucose, sorbitol, acacia, alginic acid, sodium alginate, gelatin, starch, pregelatinized starch, microcrystalline cellulose, magnesium aluminum silicate, carboxymethylcellulose sodium, methylcellulose, optionally substituted hydroxylpropyl methylcellulose, ethylcellulose, polyvinylpyrrolidone, or polyethylene glycol); and lubricating agents, glidants, and antiadhesives (e.g., magnesium stearate, zinc stearate, stearic acid, silicas, hydrogenated vegetable oils, or talc). Other pharmaceutically acceptable excipients can be colorants, flavoring agents, plasticizers, humectants, buffering agents, and the like.

Two or more compounds may be mixed together in a tablet, capsule, or other vehicle, or may be partitioned. In one example, the first compound is contained on the inside of the tablet, and the second compound is on the outside, such that a substantial portion of the second compound is released prior to the release of the first compound.

Formulations for oral use may also be provided as chewable tablets, or as hard gelatin capsules wherein the active ingredient is mixed with an inert solid diluent (e.g., potato starch, lactose, microcrystalline cellulose, calcium carbonate, calcium phosphate or kaolin), or as soft gelatin capsules wherein the active ingredient is mixed with water or an oil medium, for example, peanut oil, liquid paraffin, or olive oil. Powders, granulates, and pellets may be prepared using the ingredients mentioned above under tablets and capsules in a conventional manner using, e.g., a mixer, a fluid bed apparatus or a spray drying equipment.

Dissolution or diffusion-controlled release can be achieved by appropriate coating of a tablet, capsule, pellet, or granulate formulation of compounds, or by incorporating the compound, or a pharmaceutically acceptable salt thereof, into an appropriate matrix. A controlled release coating may include one or more of the coating substances mentioned above or, e.g., shellac, beeswax, glycowax, castor wax, carnauba wax, stearyl alcohol, glyceryl monostearate, glyceryl distearate, glycerol palmitostearate, ethylcellulose, acrylic resins, dl-polylactic acid, cellulose acetate butyrate, polyvinyl chloride, polyvinyl acetate, vinyl pyrrolidone, polyethylene, polymethacrylate, methylmethacrylate, 2-optionally substituted hydroxylmethacrylate, methacrylate hydrogels, 1,3 butylene glycol, ethylene glycol methacrylate, or polyethylene glycols. In a controlled release matrix formulation, the matrix material may also include, e.g., hydrated methylcellulose, carnauba wax and stearyl alcohol, carbopol 934, silicone, glyceryl tristearate, methyl acrylate-methyl methacrylate, polyvinyl chloride, polyethylene, or halogenated fluorocarbon.

The liquid forms in which the compounds, or a pharmaceutically acceptable salt thereof, and compositions of the present invention can be incorporated for administration orally include aqueous solutions, suitably flavored syrups, aqueous or oil suspensions, and flavored emulsions with edible oils such as cottonseed oil, sesame oil, coconut oil, or peanut oil, as well as elixirs and similar pharmaceutical vehicles.

Generally, when administered to a human, the oral dosage of any of the compounds of the invention, or a pharmaceutically acceptable salt thereof, will depend on the nature of the compound, and can readily be determined by one skilled in the art. A dosage may be, for example, about 0.001 mg to about 2000 mg per day, about 1 mg to about 1000 mg per day, about 5 mg to about 500 mg per day, about 100 mg to about 1500 mg per day, about 500 mg to about 1500 mg per day, about 500 mg to about 2000 mg per day, or any range derivable therein.

In some embodiments, the pharmaceutical composition may further comprise an additional compound having antiproliferative activity. Depending on the mode of administration, compounds, or a pharmaceutically acceptable salt thereof, will be formulated into suitable compositions to permit facile delivery. Each compound, or a pharmaceutically acceptable salt thereof, of a combination therapy may be formulated in a variety of ways that are known in the art. For example, the first and second agents of the combination therapy may be formulated together or separately. Desirably, the first and second agents are formulated together for the simultaneous or near simultaneous administration of the agents.

It will be appreciated that the compounds and pharmaceutical compositions of the present invention can be formulated and employed in combination therapies, that is, the compounds and pharmaceutical compositions can be formulated with or administered concurrently with, prior to, or subsequent to, one or more other desired therapeutics or medical procedures. The particular combination of therapies (therapeutics or procedures) to employ in a combination regimen will take into account compatibility of the desired therapeutics or procedures and the desired therapeutic effect to be achieved. It will also be appreciated that the therapies employed may achieve a desired effect for the same disorder, or they may achieve different effects (e.g., control of any adverse effects).

Administration of each drug in a combination therapy, as described herein, can, independently, be one to four times daily for one day to one year, and may even be for the life of the subject. Chronic, long-term administration may be indicated.

### Methods of Use

In some embodiments, the invention discloses a method of treating a disease or disorder that is characterized by aberrant Ras activity due to a Ras mutant. In some embodiments, the disease or disorder is a cancer.

Accordingly, also provided is a method of treating cancer in a subject in need thereof, the method comprising administering to the subject a therapeutically effective amount of a compound of the present invention, or a pharmaceutically acceptable salt thereof, or a pharmaceutical composition comprising such a compound or salt. In some embodiments, the cancer is colorectal cancer, non-small cell lung cancer, small-cell lung cancer, pancreatic cancer, appendiceal cancer, melanoma, acute myeloid leukemia, small bowel cancer, ampullary cancer, germ cell cancer, cervical cancer, cancer of unknown primary origin, endometrial cancer, esophagogastric cancer, GI neuroendocrine cancer, ovarian cancer, sex cord stromal tumor cancer, hepatobiliary cancer, or bladder cancer. In some embodiments, the cancer is appendiceal, endometrial or melanoma. Also provided is a method of treating a Ras protein-related disorder in a subject in need thereof, the method comprising administering to the subject a therapeutically effective amount of a compound of the present invention, or a pharmaceutically acceptable salt thereof, or a pharmaceutical composition comprising such a compound or salt.

In some embodiments, the compounds of the present invention or pharmaceutically acceptable salts thereof, pharmaceutical compositions comprising such compounds or salts, and methods provided herein may be used for the treatment of a wide variety of cancers including tumors such as lung, prostate, breast, brain, skin, cervical carcinomas, testicular carcinomas, etc. More particularly, cancers that may be treated by the compounds or salts thereof, pharmaceutical compositions comprising such compounds or salts, and methods of the invention include, but are not limited to tumor types such as astrocytic, breast, cervical, colorectal, endometrial, esophageal, gastric, head and neck, hepatocellular, laryngeal, lung, oral, ovarian, prostate, and thyroid carcinomas and sarcomas, Other cancers include, for example:
Cardiac, for example: sarcoma (angiosarcoma, fibrosarcoma, rhabdomyosarcoma, liposarcoma), myxoma, rhabdomyoma, fibroma, lipoma, and teratoma;
Lung, for example: bronchogenic carcinoma (squamous cell, undifferentiated small cell, undifferentiated large cell, adenocarcinoma), alveolar (bronchiolar) carcinoma, bronchial adenoma, sarcoma, lymphoma, chondromatous harnartoma, mesothelioma;
Gastrointestinal, for example: esophagus (squamous cell carcinoma, adenocarcinoma, leiomyosarcoma, lymphoma), stomach (carcinoma, lymphoma, leiomyosarcoma), pancreas (ductal adenocarcinoma, insulinoma, glucagonoma, gastrinoma, carcinoid tumors, vipoma), small bowel (adenocarcinoma, lymphoma, carcinoid tumors, Kaposi's sarcoma, leiomyoma, hemangioma, lipoma, neurofibroma, fibroma), large bowel (adenocarcinoma, tubular adenoma, villous adenoma, hamartoma, leiomyoma);
Genitourinary tract, for example: kidney (adenocarcinoma, Wilm's tumor (nephroblastoma), lymphoma, leukemia), bladder and urethra (squamous cell carcinoma, transitional cell carcinoma, adenocarcinoma), prostate (adenocarcinoma, sarcoma), testis (seminoma, teratoma, embryonal carcinoma, teratocarcinoma, choriocarcinoma, sarcoma, interstitial cell carcinoma, fibroma, fibroadenoma, adenomatoid tumors, lipoma);
Liver, for example: hepatoma (hepatocellular carcinoma), cholangiocarcinoma, hepatoblastoma, angiosarcoma, hepatocellular adenoma, hemangioma;
Biliary tract, for example: gall bladder carcinoma, ampullary carcinoma, cholangiocarcinoma;
Bone, for example: osteogenic sarcoma (osteosarcoma), fibrosarcoma, malignant fibrous histiocytoma, chondrosarcoma, Ewing's sarcoma, malignant lymphoma (reticulum cell sarcoma), multiple myeloma, malignant giant cell tumor chordoma, osteochronfroma (osteocartilaginous exostoses), benign chondroma, chondroblastoma, chondromyxofibroma, osteoid osteoma, and giant cell tumors;
Nervous system, for example: skull (osteoma, hemangioma, granuloma, xanthoma, osteitis deformans), meninges (meningioma, meningiosarcoma, gliomatosis), brain (astrocytoma, medulloblastoma, glioma, ependymoma, germinoma (pinealoma), glioblastoma multiform, oligodendroglioma, schwannoma, retinoblastoma, congenital tumors), spinal cord neurofibroma, neurofibromatosis type 1, meningioma, glioma, sarcoma);
Gynecological, for example: uterus (endometrial carcinoma, uterine carcinoma, uterine corpus endometrial carcinoma), cervix (cervical carcinoma, pre-tumor cervical dysplasia), ovaries (ovarian carcinoma (serous cystadenocarcinoma, mucinous cystadenocarcinoma, unclassified carcinoma), granulosa-thecal cell tumors, Sertoli-Leydig cell tumors, dysgerminoma, malignant teratoma), vulva (squamous cell carcinoma, intraepithelial carcinoma, adenocarcinoma, fibrosarcoma, melanoma), vagina (clear cell carcinoma, squamous cell carcinoma, botryoid sarcoma (embryonal rhabdomyosarcoma), fallopian tubes (carcinoma);
Hematologic, for example: blood (myeloid leukemia (acute and chronic), acute lymphoblastic leukemia, chronic lymphocytic leukemia, myeloproliferative diseases (e.g., myelofibrosis and myeloproliferative neoplasms), multiple myeloma, myelodysplastic syndrome), Hodgkin's disease, non-Hodgkin's lymphoma (malignant lymphoma);
Skin, for example: malignant melanoma, basal cell carcinoma, squamous cell carcinoma, Kaposi's sarcoma, moles dysplastic nevi, lipoma, angioma, dermatofibroma, keloids, psoriasis; and
Adrenal glands, for example: neuroblastoma.

In some embodiments, the Ras protein is wild type (Ras*T). Accordingly, in some embodiments, a compound of the present invention is employed in a method of treating a patient having a cancer comprising a Ras*T (e.g., K-Ras*T, H-Ras*T or N-Ras*T). In some embodiments, the Ras protein is Ras amplification (e.g., K-Ras^{amp}). Accordingly, in some embodiments, a compound of the present invention is employed in a method of treating a patient having a cancer comprising a Ras^{amp} (K-Ras^{amp}, H-Ras^{amp} or N-Ras^{amp}). In some embodiments, the cancer comprises a Ras mutation, such as a Ras mutation described herein. In some embodiments, a mutation is selected from:
(a) the following K-Ras mutants: G12D, G12V, G12C, G13D, G12R, G12A, Q61H, G12S, A146T, G13C, Q61L, Q61R, K117N, A146V, G12F, Q61K, L19F, Q22K, V14I, A59T, A146P, G13R, G12L, or G13V, and combinations thereof;
(b) the following H-Ras mutants: Q61R, G13R, Q61K, G12S, Q61L, G12D, G13V, G13D, G12C, K117N, A59T, G12V, G13C, Q61H, G13S, A18V, D119N, G13N, A146T, A66T, G12A, A146V, G12N, or G12R, and combinations thereof; and
(c) the following N-Ras mutants: Q61R, Q61K, G12D, Q61L, Q61H, G13R, G13D, G12S, G12C, G12V, G12A, G13V, G12R, P185S, G13C, A146T, G60E, Q61P, A59D, E132K, E49K, T50I, A146V, or A59T, and combinations thereof;
   or a combination of any of the foregoing. In some embodiments, the cancer comprises a K-Ras mutation selected from the group consisting of G12C, G12D, G13C, G12V, G13D, G12R, G12S, Q61H, Q61K, Q61R and Q61L. In some embodiments, the cancer comprises a K-Ras mutation that is Q61H. In some embodiments, the cancer comprises an N-Ras mutation selected from the group consisting of G12C, Q61H, Q61K, Q61 L, Q61P and Q61R. In some embodiments, the cancer comprises an H-Ras mutation selected from the group consisting of Q61H and Q61 L. In some embodiments, the cancer comprises a K-Ras mutation that is Q61H. In some embodiments, a compound of the present invention inhibits more than one Ras mutant. In some embodiments, a compound of the present invention inhibits Ras^{V} in addition to one or more additional Ras mutations (e.g., K-, H- or N-Rasv and K-Ras G12D, G12V, G12C, G13D, G12R, G12A, Q61H, G12S, A146T, G13C, Q61L, Q61R, K117N, A146V, G12F, Q61K, L19F, Q22K, V14I, A59T, A146P, G13R, G12L, or G13V; K, H or N-Rasv and H-Ras Q61R, G13R, Q61K, G12S, Q61L, G12D, G13V, G13D, G12C, K117N, A59T, G12V, G13C, Q61H, G13S, A18V, D119N, G13N, A146T, A66T, G12A, A146V, G12N, or G12R; or K, H or N-Ras^{V} and N-Ras Q61R, Q61K, G12D, Q61L, Q61H, G13R, G13D, G12S, G12C, G12V, G12A, G13V, G12R, P185S, G13C, A146T, G60E, Q61P, A59D, E132K, E49K, T50I, A146V, or A59T). In some embodiments, a compound of the present invention inhibits Ras^{amp} in addition to one or more additional Ras mutations (e.g., K-, H- or N-Ras^{amp} and K-Ras G12D, G12V, G12C, G13D, G12R, G12A, Q61H, G12S, A146T, G13C, Q61L, Q61R, K117N, A146V, G12F, Q61K, L19F, Q22K, V14I, A59T, A146P, G13R, G12L, or G13V; K, H or N-Ras^{amp} and H-Ras Q61R, G13R, Q61K, G12S, Q61L, G12D, G13V, G13D, G12C, K117N, A59T, G12V, G13C, Q61H, G13S, A18V, D119N, G13N, A146T, A66T, G12A, A146V, G12N, or G12R; or K, H or N-Ras^{amp} and N-Ras Q61R, Q61K, G12D, Q61L, Q61H, G13R, G13D, G12S, G12C, G12V, G12A, G13V, G12R, P185S, G13C, A146T, G60E, Q61P, A59D, E132K, E49K, T50I, A146V, or A59T).

Methods of detecting Ras mutations are known in the art. Such means include, but are not limited to direct sequencing, and utilization of a high-sensitivity diagnostic assay (with CE-IVD mark), e.g., as described in Domagala, et al., Pol J Pathol 3: 145-164 (2012), incorporated herein by reference in its entirety, including TheraScreen PCR; AmoyDx; PNAClamp; RealQuality; EntroGen; LightMix; StripAssay; Hybcell plexA; Devyser; Surveyor; Cobas; and TheraScreen Pyro. See, also, e.g., WO 2020/106640.

In some embodiments, a cancer comprises a Ras Q61H mutation and a TP53, an STK11^{LOF} a CDKN2A, a KEAP1, a CDKN2B, an MTAP, an RBM10, a SMARCA4, an ATM, a MYC, an APC, a SMAD4, a PIK3CA, an SOX9, an FBXW7, a PTEN, a FLT3, an AMER1, a CDK8, a AKT2, an RNF43, a GATA6, an SF381, an IGH, a CDKN2C, a DNMT3A, an RB1, a TRAF3, an N-Ras, a TET2, an FAF1, a BRAF, a KMT2A, an RUNX1, a PTPN11, a ETV6, an NPM1 or an MYH11 mutation. In some embodiments, the cancer is non-small cell lung cancer and comprises a K-Ras Q61H mutation and a TP53, an STK11^{LOF}, a CDKN2A, a KEAP1, a CDKN2B, an MTAP, an RBM10, a SMARCA4, an ATM, or a MYC mutation. In some embodiments, the cancer is colorectal cancer and comprises a K-Ras Q61H mutation and an APC, a TP53, an SMAD4, a PIK3CA, an SOX9, an FBXW7, a PTEN, a FLT3, an AMER1 or a CDK8 mutation. In some embodiments, the cancer is pancreatic cancer and comprises a K-Ras Q61H mutation and a TP53, a CDKN2A, a CDKN2B, an MTAP, an SMAD4, an ATM, an AKT2, an RNF43, a GATA6 or an SF381 mutation. In some embodiments, the cancer is multiple myeloma and comprises a K-Ras Q61H mutation and an IGH, a TP53, a CDKN2C, a DNMT3A, an RB1, a TRAF3, an N-Ras, a TET2, an FAF1 or a BRAF mutation. In some embodiments, the cancer is acute myeloid leukemia and comprises a K-Ras Q61H mutation and an N-Ras, a KMT2A, a FLT3, a DNMT3A, a RUNX1, a PTPN11, a TP53, an ETV6, an NPM1 or an MYH11 mutation. In some embodiments, the cancer is melanoma, and the Ras mutation comprises an N-Ras mutation, such as N-Ras Q61R or N-Ras Q61K. In any of the foregoing, a compound may inhibit Ras^{WT} (e.g., K-, H- or N-Ras^{WT}) or Ras^{amp} (e.g., K-, H- or N-Ras^{amp}) as well.

Also provided is a method of inhibiting a Ras protein in a cell, the method comprising contacting the cell with an effective amount of a compound of the present invention, or a pharmaceutically acceptable salt thereof. A method of inhibiting RAF-Ras binding, the method comprising contacting the cell with an effective amount of a compound of the present invention, or a pharmaceutically acceptable salt thereof, is also provided. The cell may be a cancer cell. The cancer cell may be of any type of cancer described herein. The cell may be in vivo or in vitro.

### Combination Therapy

The methods of the invention may include a compound of the invention used alone or in combination with one or more additional therapies (e.g., non-drug treatments or therapeutic agents). The dosages of one or more of the additional therapies (e.g., non-drug treatments or therapeutic agents) may be reduced from standard dosages when administered alone. For example, doses may be determined empirically from drug combinations and permutations or may be deduced by isobolographic analysis (e.g., Black et al., Neurology 65:S3-S6 (2005)).

A compound of the present invention may be administered before, after, or concurrently with one or more of such additional therapies. When combined, dosages of a compound of the invention and dosages of the one or more additional therapies (e.g., non-drug treatment or therapeutic agent) provide a therapeutic effect (e.g., synergistic or additive therapeutic effect). A compound of the present invention and an additional therapy, such as an anti-cancer agent, may be administered together, such as in a unitary pharmaceutical composition, or separately and, when administered separately, this may occur simultaneously or sequentially. Such sequential administration may be close or remote in time.

In some embodiments, the additional therapy is the administration of side-effect limiting agents (e.g., agents intended to lessen the occurrence or severity of side effects of treatment. For example, in some embodiments, the compounds of the present invention can also be used in combination with a therapeutic agent that treats nausea. Examples of agents that can be used to treat nausea include: dronabinol, granisetron, metoclopramide, ondansetron, and prochlorperazine, or pharmaceutically acceptable salts thereof.

In some embodiments, the one or more additional therapies includes a non-drug treatment (e.g., surgery or radiation therapy). In some embodiments, the one or more additional therapies includes a therapeutic agent (e.g., a compound or biologic that is an anti-angiogenic agent, signal transduction inhibitor, antiproliferative agent, glycolysis inhibitor, or autophagy inhibitor). In some embodiments, the one or more additional therapies includes a non-drug treatment (e.g., surgery or radiation therapy) and a therapeutic agent (e.g., a compound or biologic that is an anti-angiogenic agent, signal transduction inhibitor, antiproliferative agent, glycolysis inhibitor, or autophagy inhibitor). In other embodiments, the one or more additional therapies includes two therapeutic agents. In still other embodiments, the one or more additional therapies includes three therapeutic agents. In some embodiments, the one or more additional therapies includes four or more therapeutic agents.

In this Combination Therapy section, all references are incorporated by reference for the agents described, whether explicitly stated as such or not.

### Non-Drug Therapies

Examples of non-drug treatments include, but are not limited to, radiation therapy, cryotherapy, hyperthermia, surgery (e.g., surgical excision of tumor tissue), and T cell adoptive transfer (ACT) therapy.

In some embodiments, the compounds of the invention may be used as an adjuvant therapy after surgery. In some embodiments, the compounds of the invention may be used as a neo-adjuvant therapy prior to surgery.

Radiation therapy may be used for inhibiting abnormal cell growth or treating a hyperproliferative disorder, such as cancer, in a subject (e.g., mammal (e.g., human)). Techniques for administering radiation therapy are known in the art. Radiation therapy can be administered through one of several methods, or a combination of methods, including, without limitation, external-beam therapy, internal radiation therapy, implant radiation, stereotactic radiosurgery, systemic radiation therapy, radiotherapy, and permanent or temporary interstitial brachy therapy. The term “brachy therapy,” as used herein, refers to radiation therapy delivered by a spatially confined radioactive material inserted into the body at or near a tumor or other proliferative tissue disease site. The term is intended, without limitation, to include exposure to radioactive isotopes (e.g. At-211, I-131, I-125, Y-90, Re-186, Re-188, Sm-153, Bi-212, P-32, and radioactive isotopes of Lu). Suitable radiation sources for use as a cell conditioner of the present invention include both solids and liquids. By way of non-limiting example, the radiation source can be a radionuclide, such as I-125, I-131, Yb-169, Ir-192 as a solid source, I-125 as a solid source, or other radionuclides that emit photons, beta particles, gamma radiation, or other therapeutic rays. The radioactive material can also be a fluid made from any solution of radionuclide(s), e.g., a solution of I-125 or I-131, or a radioactive fluid can be produced using a slurry of a suitable fluid containing small particles of solid radionuclides, such as Au-198, or Y-90. Moreover, the radionuclide(s) can be embodied in a gel or radioactive micro spheres.

In some embodiments, the compounds of the present invention can render abnormal cells more sensitive to treatment with radiation for purposes of killing or inhibiting the growth of such cells. Accordingly, this invention further relates to a method for sensitizing abnormal cells in a mammal to treatment with radiation which comprises administering to the mammal an amount of a compound of the present invention, which amount is effective to sensitize abnormal cells to treatment with radiation. The amount of the compound in this method can be determined according to the means for ascertaining effective amounts of such compounds described herein. In some embodiments, the compounds of the present invention may be used as an adjuvant therapy after radiation therapy or as a neo-adjuvant therapy prior to radiation therapy.

In some embodiments, the non-drug treatment is a T cell adoptive transfer (ACT) therapy. In some embodiments, the T cell is an activated T cell. The T cell may be modified to express a chimeric antigen receptor (CAR). CAR modified T (CAR-T) cells can be generated by any method known in the art. For example, the CAR-T cells can be generated by introducing a suitable expression vector encoding the CAR to a T cell. Prior to expansion and genetic modification of the T cells, a source of T cells is obtained from a subject. T cells can be obtained from a number of sources, including peripheral blood mononuclear cells, bone marrow, lymph node tissue, cord blood, thymus tissue, tissue from a site of infection, ascites, pleural effusion, spleen tissue, and tumors. In certain embodiments of the present invention, any number of T cell lines available in the art may be used. In some embodiments, the T cell is an autologous T cell. Whether prior to or after genetic modification of the T cells to express a desirable protein (e.g., a CAR), the T cells can be activated and expanded generally using methods as described, for example, in U.S. Pat. Nos. 6,352,694; 6,534,055; 6,905,680; 6,692,964; 5,858,358; 6,887,466; 6,905,681; 7,144,575; 7,067,318; 7,172,869; 7,232,566; 7,175,843; 7,572,631; 5,883,223; 6,905,874; 6,797,514; and 6,867,041.

### Therapeutic Agents

A therapeutic agent may be a compound used in the treatment of cancer or symptoms associated therewith. A compound of the present invention may be combined with a second, third, or fourth therapeutic agent, or more. A compound of the present invention may be combined with one or more therapeutic agents along with one or more non-drug therapies.

For example, a therapeutic agent may be a steroid. Steroids are known in the art. Accordingly, in some embodiments, the one or more additional therapies includes a steroid. Suitable steroids may include, but are not limited to, 21-acetoxypregnenolone, alclometasone, algestone, amcinonide, beclomethasone, betamethasone, budesonide, chloroprednisone, clobetasol, clocortolone, cloprednol, corticosterone, cortisone, cortivazol, deflazacort, desonide, desoximetasone, dexamethasone, diflorasone, diflucortolone, difuprednate, enoxolone, fluazacort, flucloronide, flumethasone, flunisolide, fluocinolone acetonide, fluocinonide, fluocortin butyl, fluocortolone, fluorometholone, fluperolone acetate, fluprednidene acetate, fluprednisolone, flurandrenolide, fluticasone propionate, formocortal, halcinonide, halobetasol propionate, halometasone, hydrocortisone, loteprednol etabonate, mazipredone, medrysone, meprednisone, methylprednisolone, mometasone furoate, paramethasone, prednicarbate, prednisolone, prednisolone 25-diethylaminoacetate, prednisolone sodium phosphate, prednisone, prednival, prednylidene, rimexolone, tixocortol, triamcinolone, triamcinolone acetonide, triamcinolone benetonide, triamcinolone hexacetonide, and salts or derivatives thereof.

Further examples of therapeutic agents that may be used in combination therapy with a compound of the present invention include compounds described in the following patents: U.S. Pat. Nos. 6,258,812, 6,630,500, 6,515,004, 6,713,485, 5,521,184, 5,770,599, 5,747,498, 5,990,141, 6,235,764, and 8,623,885, and International Patent Applications WO01/37820, WO01/32651, WO02/68406, WO02/66470, WO02/55501, WO04/05279, WO04/07481, WO04/07458, WO04/09784, WO02/59110, WO99/45009, WO00/59509, WO99/61422, WO00/12089, and WO00/02871.

A therapeutic agent may be a biologic (e.g., cytokine (e.g., interferon or an interleukin such as IL-2)) used in treatment of cancer or symptoms associated therewith. Biologics are known in the art. In some embodiments, the biologic is an immunoglobulin-based biologic, e.g., a monoclonal antibody (e.g., a humanized antibody, a fully human antibody, an Fc fusion protein, or a functional fragment thereof) that agonizes a target to stimulate an anti-cancer response or antagonizes an antigen important for cancer. Also included are antibody-drug conjugates.

A therapeutic agent may be a T-cell checkpoint inhibitor. Such checkpoint inhibitors are known in the art. In one embodiment, the checkpoint inhibitor is an inhibitory antibody (e.g., a monospecific antibody such as a monoclonal antibody). The antibody may be, e.g., humanized or fully human. In some embodiments, the checkpoint inhibitor is a fusion protein, e.g., an Fc-receptor fusion protein. In some embodiments, the checkpoint inhibitor is an agent, such as an antibody, that interacts with a checkpoint protein. In some embodiments, the checkpoint inhibitor is an agent, such as an antibody, that interacts with the ligand of a checkpoint protein. In some embodiments, the checkpoint inhibitor is an inhibitor (e.g., an inhibitory antibody or small molecule inhibitor) of CTLA-4 (e.g., an anti-CTLA-4 antibody or fusion a protein). In some embodiments, the checkpoint inhibitor is an inhibitor or antagonist (e.g., an inhibitory antibody or small molecule inhibitor) of PD-1. In some embodiments, the checkpoint inhibitor is an inhibitor or antagonist (e.g., an inhibitory antibody or small molecule inhibitor) of PD-L1. In some embodiments, the checkpoint inhibitor is an inhibitor or antagonist (e.g., an inhibitory antibody or Fc fusion or small molecule inhibitor) of PD-L2 (e.g., a PD-L2/Ig fusion protein). In some embodiments, the checkpoint inhibitor is an inhibitor or antagonist (e.g., an inhibitory antibody or small molecule inhibitor) of B7-H3, B7-H4, BTLA, HVEM, TIM3, GAL9, LAG3, VISTA, KIR, 2B4, CD160, CGEN-15049, CHK 1, CHK2, A2aR, B-7 family ligands, or a combination thereof. In some embodiments, the checkpoint inhibitor is pembrolizumab, nivolumab, PDR001 (NVS), REGN2810 (Sanofi/Regeneron), a PD-L1 antibody such as, e.g., avelumab, durvalumab, atezolizumab, pidilizumab, JNJ-63723283 (JNJ), BGB-A317 (BeiGene & Celgene) or a checkpoint inhibitor disclosed in Preusser, M. et al. (2015) Nat. Rev. Neurol., including, without limitation, ipilimumab, tremelimumab, nivolumab, pembrolizumab, AMP224, AMP514/MEDI0680, BMS936559, MEDI4736, MPDL3280A, MSB0010718C, BMS986016, IMP321, lirilumab, IPH2101, 1-7F9, and KW-6002.

A therapeutic agent may be an anti-TIGIT antibody, such as MBSA43, BMS-986207, MK-7684, COM902, AB154, MTIG7192A or OMP-313M32 (etigilimab). Other anti-TIGIT antibodies are known in the art.

A therapeutic agent may be an agent that treats cancer or symptoms associated therewith (e.g., a cytotoxic agent, non-peptide small molecules, or other compound useful in the treatment of cancer or symptoms associated therewith, collectively, an “anti-cancer agent”). Anti-cancer agents can be, e.g., chemotherapeutics or targeted therapy agents. Such agents are known in the art.

Anti-cancer agents include mitotic inhibitors, intercalating antibiotics, growth factor inhibitors, cell cycle inhibitors, enzymes, topoisomerase inhibitors, biological response modifiers, alkylating agents, antimetabolites, folic acid analogs, pyrimidine analogs, purine analogs and related inhibitors, vinca alkaloids, epipodopyyllotoxins, antibiotics, L-Asparaginase, topoisomerase inhibitors, interferons, platinum coordination complexes, anthracenedione substituted urea, methyl hydrazine derivatives, adrenocortical suppressant, adrenocorticosteroides, progestins, estrogens, antiestrogen, androgens, antiandrogen, and gonadotropin-releasing hormone analog. Further anti-cancer agents include leucovorin (LV), irenotecan, oxaliplatin, capecitabine, paclitaxel, and doxetaxel. In some embodiments, the one or more additional therapies includes two or more anti-cancer agents. The two or more anti-cancer agents can be used in a cocktail to be administered in combination or administered separately. Suitable dosing regimens of combination anti-cancer agents are known in the art and described in, for example, Saltz et al., Proc. Am. Soc. Clin. Oncol. 18:233a (1999), and Douillard et al., Lancet 355(9209):1041-1047 (2000).

Other non-limiting examples of anti-cancer agents include Gleevec® (Imatinib Mesylate); Kyprolis® (carfilzomib); Velcade® (bortezornib); Casodex (bicalutamide); Iressa® (gefitinib); alkylating agents such as thiotepa and cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, triethylenephosphoramide, triethiylenethiophosphoramide and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CB1-TM1); eleutherobin; pancratistatin; sarcodictyin A; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimustine; antibiotics such as the enediyne antibiotics (e.g., calicheamicin, such as calicheamicin gammall and calicheamicin omegall (see, e.g., Agnew, Chem. Intl. Ed Engl. 33:183-186 (1994)); dynemicin such as dynemicin A; bisphosphonates such as clodronate; an esperamicin; neocarzinostatin chromophore and related chromoprotein enediyne antiobiotic chromophores, aclacinomysins, actinomycin, authramycin, azaserine, bleomycins, cactinomycin, calicheamicin, carabicin, caminomycin, carminomycin, carzinophilin, chromomycins, dactinomycin, daunorubicin, detorubicin, 6-diazo-5-oxo-L-norleucine, adriamycin (doxorubicin), morpholino-doxorubicin, cyanomorpholino-doxorubicin, 2-pyrrolino-doxorubicin, deoxydoxorubicin, epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenishers such as frolinic acid; aceglatone; aldophosphamide glycoside; aminolevulinic acid; eniluracil; amsacrine; bestrabucil; bisantrene; edatraxate; defofamine; demecolcine; diaziquone; elfomithine; elliptinium acetate; an epothilone such as epothilone B; etoglucid; gallium nitrate; hydroxyurea; lentinan; lonidamine; maytansinoids such as maytansine and ansamitocins; mitoguazone; mitoxantrone; mopidamol; nitracrine; pentostatin; phenamet; pirarubicin; losoxantrone; podophyllinic acid; 2-ethylhydrazide; procarbazine; PSK® polysaccharide complex (JHS Natural Products, Eugene, OR); razoxane; rhizoxin; sizofiran; spirogermanium; tenuazonic acid; triaziquone; 2,2′,2″-trichlorotriethylamine; trichothecenes such as T-2 toxin, verracurin A, roridin A and anguidine; urethane; vindesine; dacarbazine; mannomustine; mitobronitol; mitolactol; pipobroman; gacytosine; arabinoside (“Ara-C”); cyclophosphamide; thiotepa; taxoids, e.g., Taxol® (paclitaxel), Abraxane® (cremophor-free, albumin-engineered nanoparticle formulation of paclitaxel), and Taxotere® (doxetaxel); chloranbucil; tamoxifen (Nolvadex™); raloxifene; aromatase inhibiting 4(5)-imidazoles; 4-hydroxytarnoxifen; trioxifene; keoxifene; LY 117018; onapristone; toremifene (Fareston®); flutamide, nilutamide, bicalutamide, leuprolide, goserelin; chlorambucil; Gemzar® gemcitabine; 6-thioguanine; mercaptopurine; platinum coordination complexes such as cisplatin, oxaliplatin and carboplatin; vinblastine; platinum; etoposide (VP-16); ifosfamide; mitoxantrone; vincristine; Navelbine® (vinorelbine); novantrone; teniposide; edatrexate; daunomycin; aminopterin; ibandronate; irinotecan (e.g., CPT-11); topoisomerase inhibitor RFS 2000; difluoromethylornithine (DMFO); retinoids such as retinoic acid; esperamicins; capecitabine (e.g., Xeloda®); and pharmaceutically acceptable salts of any of the above.

Additional non-limiting examples of anti-cancer agents include trastuzumab (Herceptin®), bevacizumab (Avastin®), cetuximab (Erbitux®), rituximab (Rituxan®), Taxol®, Arimidex®, ABVD, avicine, abagovomab, acridine carboxamide, adecatumumab, 17-N-allylamino-17-demethoxygeldanamycin, alpharadin, alvocidib, 3-aminopyridine-2-carboxaldehyde thiosemicarbazone, amonafide, anthracenedione, anti-CD22 immunotoxins, antineoplastics (e.g., cell-cycle nonspecific antineoplastic agents, and other antineoplastics described herein), antitumorigenic herbs, apaziquone, atiprimod, azathioprine, belotecan, bendamustine, BIBW 2992, biricodar, brostallicin, bryostatin, buthionine sulfoximine, CBV (chemotherapy), calyculin, dichloroacetic acid, discodermolide, elsamitrucin, enocitabine, eribulin, exatecan, exisulind, ferruginol, forodesine, fosfestrol, ICE chemotherapy regimen, IT-101, imexon, imiquimod, indolocarbazole, irofulven, Ianiquidar, larotaxel, lenalidomide, lucanthone, lurtotecan, mafosfamide, mitozolomide, nafoxidine, nedaplatin, olaparib, ortataxel, PAC-1, pawpaw, pixantrone, proteasome inhibitors, rebeccamycin, resiquimod, rubitecan, SN-38, salinosporamide A, sapacitabine, Stanford V, swainsonine, talaporfin, tariquidar, tegafur-uracil, temodar, tesetaxel, triplatin tetranitrate, tris(2-chloroethyl)amine, troxacitabine, uramustine, vadimezan, vinflunine, ZD6126, and zosuquidar.

Further non-limiting examples of anti-cancer agents include natural products such as vinca alkaloids (e.g., vinblastine, vincristine, and vinorelbine), epidipodophyllotoxins (e.g., etoposide and teniposide), antibiotics (e.g., dactinomycin (actinomycin D), daunorubicin, and idarubicin), anthracyclines, mitoxantrone, bleomycins, plicamycin (mithramycin), mitomycin, enzymes (e.g., L-asparaginase which systemically metabolizes L-asparagine and deprives cells which do not have the capacity to synthesize their own asparagine), antiplatelet agents, antiproliferative/antimitotic alkylating agents such as nitrogen mustards (e.g., mechlorethamine, cyclophosphamide and analogs, melphalan, and chlorambucil), ethylenimines and methylmelamines (e.g., hexamethylmelamine and thiotepa), CDK inhibitors (e.g., a CDK4/6 inhibitor such as abemaciclib, ribociclib, palbociclib; seliciclib, UCN-01, P1446A-05, PD-0332991, dinaciclib, P27-00, AT-7519, RGB286638, and SCH727965), alkyl sulfonates (e.g., busulfan), nitrosoureas (e.g., carmustine (BCNU) and analogs, and streptozocin), trazenes-dacarbazinine (DTIC), antiproliferative/antimitotic antimetabolites such as folic acid analogs, pyrimidine analogs (e.g., fluorouracil, floxuridine, and cytarabine), purine analogs and related inhibitors (e.g., mercaptopurine, thioguanine, pentostatin, and 2-chlorodeoxyadenosine), aromatase inhibitors (e.g., anastrozole, exemestane, and letrozole), and platinum coordination complexes (e.g., cisplatin and carboplatin), procarbazine, hydroxyurea, mitotane, aminoglutethimide, histone deacetylase (HDAC) inhibitors (e.g., trichostatin, sodium butyrate, apicidan, suberoyl anilide hydroamic acid, vorinostat, belinostat, LBH 589, romidepsin, ACY-1215, and panobinostat), mTOR inhibitors (e.g., vistusertib, temsirolimus, everolimus, ridaforolimus, and sirolimus), KSP(Eg5) inhibitors (e.g., Array 520), DNA binding agents (e.g., Zalypsis®), PI3K inhibitors such as PI3K delta inhibitor (e.g., GS-1101 and TGR-1202), PI3K delta and gamma inhibitor (e.g., CAL-130), copanlisib, alpelisib and idelalisib; multi-kinase inhibitor (e.g., TG02 and sorafenib), hormones (e.g., estrogen) and hormone agonists such as leutinizing hormone releasing hormone (LHRH) agonists (e.g., goserelin, leuprolide and triptorelin), BAFF-neutralizing antibody (e.g., LY2127399), IKK inhibitors, p38MAPK inhibitors, anti-IL-6 (e.g., CNT0328), telomerase inhibitors (e.g., GRN 163L), aurora kinase inhibitors (e.g., MLN8237), cell surface monoclonal antibodies (e.g., anti-CD38 (HUMAX-CD38), anti-CSI (e.g., elotuzumab), HSP90 inhibitors (e.g., 17 AAG and KOS 953), PI3K/Akt inhibitors (e.g., perifosine), Akt inhibitors (e.g., GSK-2141795), PKC inhibitors (e.g., enzastaurin), FTIs (e.g., Zarnestra™), anti-CD138 (e.g., BT062), Torcl/2 specific kinase inhibitors (e.g., INK128), ER/UPR targeting agents (e.g., MKC-3946), cFMS inhibitors (e.g., ARRY-382), JAK1/2 inhibitors (e.g., CYT387), PARP inhibitors (e.g., olaparib and veliparib (ABT-888)), and BCL-2 antagonists.

In some embodiments, an anti-cancer agent is selected from mechlorethamine, camptothecin, ifosfamide, tamoxifen, raloxifene, gemcitabine, Navelbine®, sorafenib, or any analog or derivative variant of the foregoing.

In some embodiments, the anti-cancer agent is a HER2 inhibitor. HER2 inhibitors are known in the art. Non-limiting examples of HER2 inhibitors include monoclonal antibodies such as trastuzumab (Herceptin®) and pertuzumab (Perjeta®); small molecule tyrosine kinase inhibitors such as gefitinib (Iressa®), erlotinib (Tarceva®), pilitinib, CP-654577, CP-724714, canertinib (CI 1033), HKI-272, lapatinib (GW-572016; Tykerb®), PKI-166, AEE788, BMS-599626, HKI-357, BIBW 2992, ARRY-334543, and JNJ-26483327.

In some embodiments, an anti-cancer agent is an ALK inhibitor. ALK inhibitors are known in the art. Non-limiting examples of ALK inhibitors include ceritinib, TAE-684 (NVP-TAE694), PF02341066 (crizotinib or 1066), alectinib; brigatinib; entrectinib; ensartinib (X-396); lorlatinib; ASP3026; CEP-37440; 4SC-203; TL-398; PLB1003; TSR-011; CT-707; TPX-0005, and AP26113. Additional examples of ALK kinase inhibitors are described in examples 3-39 of WO05016894.

In some embodiments, an anti-cancer agent is an inhibitor of a member downstream of a Receptor Tyrosine Kinase (RTK)/Growth Factor Receptor (e.g., a SHP2 inhibitor (e.g., SHP099, TNO155, RMC-4550, RMC-4630, JAB-3068, JAB-3312, RLY-1971, ERAS-601, SH3809, PF-07284892, or BBP-398), or a pharmaceutically acceptable salt, solvate, isomer (e.g., stereoisomer), prodrug, or tautomer thereof), an SOS1 inhibitor (e.g., BI-1701963, BI-3406, SDR5, BAY-293, MRTX-0902, or RMC-5845, or a pharmaceutically acceptable salt, solvate, isomer (e.g., stereoisomer), prodrug, or tautomer thereof), a Raf inhibitor, a MEK inhibitor, an ERK inhibitor, a PI3K inhibitor, a PTEN inhibitor, an AKT inhibitor, or an mTOR inhibitor (e.g., mTORC1 inhibitor or mTORC2 inhibitor). In some embodiments, the anti-cancer agent is JAB-3312.

In some embodiments, an anti-cancer agent is a SOS1 inhibitor. SOS1 inhibitors are known in the art. In some embodiments, the SOS1 inhibitor is selected from those disclosed in WO 2022219035, WO 2022214594, WO 2022199670, WO 2022146698, WO 2022081912, WO 2022058344, WO 2022026465, WO 2022017519, WO 2021173524, WO 2021130731, WO 2021127429, WO 2021092115, WO 2021105960, WO 2021074227, WO 2020180768, WO 2020180770, WO 2020173935, WO 2020146470, WO 2019201848, WO 2019122129, WO 2018172250, and WO 2018115380, or a pharmaceutically acceptable salt, solvate, isomer (e.g., stereoisomer), prodrug, or tautomer thereof.

In some embodiments, an anti-cancer agent is an additional Ras inhibitor or a Ras vaccine, or another therapeutic modality designed to directly or indirectly decrease the oncogenic activity of Ras. Such agents are known in the art. In some embodiments, an anti-cancer agent is an additional Ras inhibitor. In some embodiments, the Ras inhibitor targets Ras in its active, or GTP-bound state. In some embodiments, the Ras inhibitor targets Ras in its inactive, or GDP-bound state. In some embodiments, the Ras inhibitor is, such as an inhibitor of K-Ras G12C, such as AMG 510, MRTX1257, MRTX849, JNJ-74699157, LY3499446, ARS-1620, ARS-853, BPI-421286, LY3537982, JDQ443, JAB-3312, JAB-21822, JAB-21000, IB1351, ERAS-3490, RMC-6291, BI 1823911, D-1553, D3S-001, HBI-2438, HS-10370, MK-1084, YL-15293, BBO-8520 (ON/OFF inhibitor), FMC-376 (ON/OFF inhibitor), GEC255, or GDC-6036. In some embodiments, the Ras inhibitor is an inhibitor of K-Ras G12D, such as MRTX1133, JAB-22000, MRTX282, ERAS-4, HRS-4642, BI-2852, ASP3082, TH-Z827, TH-7835, RMC-9805, GFH375 (VS-7375), INCB161734, and KD-8. In some embodiments, the Ras inhibitor is a K-Ras G12V inhibitor, such as JAB-23000. In some embodiments, the KRAS(OFF) inhibitor is a pan-KRAS(OFF) inhibitor. In specific embodiments, the pan-KRAS(OFF) inhibitor is JAB-23400, JAB-23425, BI-2493, BI-2865, QTX-3034 (G12D preferring), QTX3544 (G12V preferring), ZG2001, BBO-a, BBO—B, or Pan KRas-IN-1. In some embodiments, the Ras inhibitor is JAB-23400. In some embodiments, the Ras inhibitor is RMC-6236. In some embodiments, the Ras inhibitor is selected from a Ras(ON) inhibitor (that is, Ras in its GTP-bound state) disclosed in the following, incorporated herein by reference in their entireties, or a pharmaceutically acceptable salt, solvate, isomer (e.g., stereoisomer), prodrug, or tautomer thereof: WO 2022/235870, WO 2022/235864, WO 2022/060836, WO 2021091982, WO 2021091967, WO 2021091956, and WO 2020132597. Other examples of Ras inhibitors are known in the art, such as in the following, incorporated herein by reference in their entireties: WO 2023287896, WO 2023287730, WO 2023284881, WO 2023284730, WO 2023284537, WO 2023283933, WO 2023283213, WO 2023280960, WO 2023280280, WO2023278600, WO 2023280136, WO 2023280026, WO 2023278600, WO 2023274383, WO 2023274324, WO 2023034290, WO 2023020523, WO 2023020521, WO 2023020519, WO 2023020518, WO 2023018812, WO 2023018810, WO 2023018809, WO 2023018699, WO 2023015559, WO 2023014979, WO 2023014006, WO 2023010121, WO 2023009716, WO 2023009572, WO 2023004102, WO 2023003417, WO 2023001141, WO 2023001123, WO 2022271923, WO 2022271823, WO 2022271810, WO 2022271658, WO 2022269508, WO 2022266167, WO 2022266069, WO 2022266015, WO 2022265974, WO 2022261154, WO 2022261154, WO 2022251576, WO 2022251296, WO 2022237815, WO 2022232332, WO 2022232331, WO 2022232320, WO 2022232318, WO 2022223037, WO 2022221739, WO 2022221528, WO 2022221386, WO 2022216762, WO 2022192794, WO 2022192790, WO 2022188729, WO 2022187411, WO 2022184178, WO 2022173870, WO 2022173678, WO 2022135346, WO 2022133731, WO 2022133038, WO 2022133345, WO 2022132200, WO 2022119748, WO 2022109485, WO 2022109487, WO 2022066805, WO 2022002102, WO 2022002018, WO 2021259331, WO 2021257828, WO 2021252339, WO 2021248095, WO 2021248090, WO 2021248083, WO 2021248082, WO 2021248079, WO 2021248055, WO 2021245051, WO 2021244603, WO 2021239058, WO 2021231526, WO 2021228161, WO 2021219090, WO 2021219090, WO 2021219072, WO 2021218939, WO 2021217019, WO 2021216770, WO 2021215545, WO 2021215544, WO 2021211864, WO 2021190467, WO 2021185233, WO 2021180181, WO 2021175199, 2021173923, WO 2021169990, WO 2021169963, WO 2021168193, WO 2021158071, WO 2021155716, WO 2021152149, WO 2021150613, WO 2021147967, WO 2021147965, WO 2021143693, WO 2021142252, WO 2021141628, WO 2021139748, WO 2021139678, WO 2021129824, WO 2021129820, WO 2021127404, WO 2021126816, WO 2021126799, WO 2021124222, WO 2021121371, WO 2021121367, WO 2021121330, WO 2020050890, WO 2020047192, WO 2020035031, WO 2020028706, WO 2019241157, WO 2019232419, WO 2019217691, WO 2019217307, WO 2019215203, WO 2019213526, WO 2019213516, WO 2019155399, WO 2019150305, WO 2019110751, WO 2019099524, WO 2019051291, WO 2018218070, WO 2018217651, WO 2018218071, WO 2018218069, WO 2018206539, WO 2018143315, WO 2018140600, WO 2018140599, WO 2018140598, WO 2018140514, WO 2018140513, WO 2018140512, WO 2018119183, WO 2018112420, WO 2018068017, WO 2018064510, WO 2017201161, WO 2017172979, WO 2017100546, WO 2017087528, WO 2017058807, WO 2017058805, WO 2017058728, WO 2017058902, WO 2017058792, WO 2017058768, WO 2017058915, WO 2017015562, WO 2016168540, WO 2016164675, WO 2016049568, WO 2016049524, WO 2015054572, WO 2014152588, WO 2014143659 and WO 2013155223.

In some embodiments, a therapeutic agent that may be combined with a compound of the present invention is an inhibitor of the MAP kinase (MAPK) pathway (or “MAPK inhibitor”). Such agents are known in the art. MAPK inhibitors include, but are not limited to, one or more MAPK inhibitor described in Cancers (Basel) 2015 September; 7(3): 1758-1784. For example, the MAPK inhibitor may be selected from one or more of trametinib, binimetinib, selumetinib, cobimetinib, LErafAON (NeoPharm), ISIS 5132; vemurafenib, pimasertib, TAK733, RO4987655 (CH4987655); CI-1040; PD-0325901; CH5126766; MAP855; AZD6244; refametinib (RDEA 119/BAY 86-9766); GDC-0973/XL581; AZD8330 (ARRY-424704/ARRY-704); RO5126766 (Roche, described in PLoS One. 2014 Nov. 25; 9(11)); and GSK1120212 (or JTP-74057, described in Clin Cancer Res. 2011 Mar. 1; 17(5):989-1000). The MAPK inhibitor may be PLX8394, LXH254, GDC-5573, or LY3009120.

In some embodiments, an anti-cancer agent is a disrupter or inhibitor of the RAS-RAF-ERK or PI3K-AKT-TOR or PI3K-AKT signaling pathways. Such agents are known in the art. The PI3K/AKT inhibitor may include, but is not limited to, one or more PI3K/AKT inhibitor described in Cancers (Basel) 2015 September; 7(3): 1758-1784. For example, the PI3K/AKT inhibitor may be selected from one or more of NVP-BEZ235; BGT226; XL765/SAR245409; SF1126; GDC-0980; PI-103; PF-04691502; PKI-587; GSK2126458.

In some embodiments, an anti-cancer agent is a PD-1 or PD-L1 antagonist. Such agents are known in the art.

In some embodiments, additional therapeutic agents include ALK inhibitors, HER2 inhibitors, EGFR inhibitors, IGF-1R inhibitors, MEK inhibitors, PI3K inhibitors, AKT inhibitors, TOR inhibitors, MCL-1 inhibitors, BCL-2 inhibitors, SHP2 inhibitors, proteasome inhibitors, and immune therapies. In some embodiments, additional therapeutic agents include FGFR inhibitors, PARP inhibitors, BET inhibitors, PRMT5i inhibitors, MAT2A inhibitors, VEGF inhibitors, and HDAC inhibitors. In some embodiments, a therapeutic agent may be a pan-RTK inhibitor, such as afatinib.

IGF-1R inhibitors are known in the art and include linsitinib, or a pharmaceutically acceptable salt thereof.

EGFR inhibitors are known in the art and include, but are not limited to, small molecule antagonists, antibody inhibitors, or specific antisense nucleotide or siRNA. Useful antibody inhibitors of EGFR include cetuximab (Erbitux®), panitumumab (Vectibix®), zalutumumab, nimotuzumab, and matuzumab. Further antibody-based EGFR inhibitors include any anti-EGFR antibody or antibody fragment that can partially or completely block EGFR activation by its natural ligand. Non-limiting examples of antibody-based EGFR inhibitors include those described in Modjtahedi et al., Br. J. Cancer 1993, 67:247-253; Teramoto et al., Cancer 1996, 77:639-645; Goldstein et al., Clin. Cancer Res. 1995, 1:1311-1318; Huang et al., 1999, Cancer Res. 15:59(8):1935-40; and Yang et al., Cancer Res. 1999, 59:1236-1243. The EGFR inhibitor can be monoclonal antibody Mab E7.6.3 (Yang, 1999 supra), or Mab C225 (ATCC Accession No. HB-8508), or an antibody or antibody fragment having the binding specificity thereof.

Small molecule antagonists of EGFR include gefitinib (Iressa®), erlotinib (Tarceva®), and lapatinib (TykerB®). See, e.g., Yan et al., Pharmacogenetics and Pharmacogenomics in Oncology Therapeutic Antibody Development, BioTechniques 2005, 39(4):565-8; and Paez et al., EGFR Mutations in Lung Cancer Correlation with Clinical Response to Gefitinib Therapy, Science 2004, 304(5676):1497-500. In some embodiments, the EGFR inhibitor is osimertinib (Tagrisso®). Further non-limiting examples of small molecule EGFR inhibitors include any of the EGFR inhibitors described in the following patent publications, and all pharmaceutically acceptable salts of such EGFR inhibitors: EP 0520722; EP 0566226; WO96/33980; U.S. Pat. No. 5,747,498; WO96/30347; EP 0787772; WO97/30034; WO97/30044; WO97/38994; WO97/49688; EP 837063; WO98/02434; WO97/38983; WO95/19774; WO95/19970; WO97/13771; WO98/02437; WO98/02438; WO97/32881; DE 19629652; WO98/33798; WO97/32880; WO97/32880; EP 682027; WO97/02266; WO97/27199; WO98/07726; WO97/34895; WO96/31510; WO98/14449; WO98/14450; WO98/14451; WO95/09847; WO97/19065; WO98/17662; U.S. Pat. Nos. 5,789,427; 5,650,415; 5,656,643; WO99/35146; WO99/35132; WO99/07701; and WO92/20642. Additional non-limiting examples of small molecule EGFR inhibitors include any of the EGFR inhibitors described in Traxler et al., Exp. Opin. Ther. Patents 1998, 8(12):1599-1625. In some embodiments, an EGFR inhibitor is an ERBB inhibitor. In humans, the ERBB family contains HER1 (EGFR, ERBB1), HER2 (NEU, ERBB2), HER3 (ERBB3), and HER (ERBB4).

MEK inhibitors are known in the art and include, but are not limited to, pimasertib, selumetinib, cobimetinib (Cotellic®), trametinib (Mekinist®), and binimetinib (Mektovi®). In some embodiments, a MEK inhibitor targets a MEK mutation that is a Class I MEK1 mutation selected from D67N; P124L; P124S; and L177V. In some embodiments, the MEK mutation is a Class II MEK1 mutation selected from ΔE51-Q58; ΔF53-Q58; E203K; L177M; C121S; F53L; K57E; Q56P; and K57N.

PI3K inhibitors are known in the art and include, but are not limited to, wortmannin; 17-hydroxywortmannin analogs described in WO06/044453; 4-[2-(1H-Indazol-4-yl)-6-[[4-(methylsulfonyl)piperazin-1-yl]methyl]thieno[3,2-d]pyrimidin-4-yl]morpholine (also known as pictilisib or GDC-0941 and described in WO09/036082 and WO09/055730); 2-methyl-2-[4-[3-methyl-2-oxo-8-(quinolin-3-yl)-2,3-dihydroimidazo[4,5-c]quinolin-1-yl]phenyl]propionitrile (also known as BEZ 235 or NVP-BEZ 235, and described in WO06/122806); (S)-1-(4-((2-(2-aminopyrimidin-5-yl)-7-methyl-4-morpholinothieno[3,2-d]pyrimidin-6-yl)methyl)piperazin-1-yl)-2-hydroxypropan-1-one (described in WO08/070740); LY294002 (2-(4-morpholinyl)-8-phenyl-4H-1-benzopyran-4-one (available from Axon Medchem); PI 103 hydrochloride (3-[4-(4-morpholinylpyrido-[3′,2′:4,5]furo[3,2-d]pyrimidin-2-yl]phenol hydrochloride (available from Axon Medchem); PIK 75 (2-methyl-5-nitro-2-[(6-bromoimidazo[1,2-a]pyridin-3-yl)methylene]-1-methylhydrazide-benzenesulfonic acid, monohydrochloride) (available from Axon Medchem); PIK 90 (N-(7,8-dimethoxy-2,3-dihydro-imidazo[1,2-c]quinazolin-5-yl)-nicotinamide (available from Axon Medchem); AS-252424 (5-[I-[5-(4-fluoro-2-hydroxy-phenyl)-furan-2-yl]-meth-(Z)-ylidene]-thiazolidine-2,4-dione (available from Axon Medchem); TGX-221 (7-methyl-2-(4-morpholinyl)-9-[1-(phenylamino)ethyl]-4H-pyrido-[1,2-a]pyrirnidin-4-one (available from Axon Medchem); XL-765; and XL-147. Other PI3K inhibitors include demethoxyviridin, perifosine, CAL101, PX-866, BEZ235, SF1126, INK1117, IPI-145, BKM120, XL147, XL765, Palomid 529, GSK1059615, ZSTK474, PWT33597, IC87114, TGI 00-115, CAL263, PI-103, GNE-477, CUDC-907, and AEZS-136.

AKT inhibitors are known in the art and include, but are not limited to, Akt-1-1 (inhibits Aktl) (Barnett et al., Biochem. J. 2005, 385(Pt. 2): 399-408); Akt-1-1,2 (inhibits Akl and 2) (Barnett et al., Biochem. J. 2005, 385(Pt. 2): 399-408); API-59CJ-Ome (e.g., Jin et al., Br. J. Cancer 2004, 91:1808-12); 1-H-imidazo[4,5-c]pyridinyl compounds (e.g., WO 05/011700); indole-3-carbinol and derivatives thereof (e.g., U.S. Pat. No. 6,656,963; Sarkar and Li J Nutr. 2004, 134(12 Suppl):3493S-3498S); perifosine (e.g., interferes with Akt membrane localization; Dasmahapatra et al. Clin. Cancer Res. 2004, 10(15):5242-52); phosphatidylinositol ether lipid analogues (e.g., Gills and Dennis Expert. Opin. Investig. Drugs 2004, 13:787-97); and triciribine (TCN or API-2 or NCI identifier: NSC 154020; Yang et al., Cancer Res. 2004, 64:4394-9).

mTOR inhibitors are known in the art and include, but are not limited to, ATP-competitive mTORC1/mTORC2 inhibitors, e.g., PI-103, PP242, PP30; Torin 1; FKBP12 enhancers; 4H-1-benzopyran-4-one derivatives; and rapamycin (also known as sirolimus) and derivatives thereof, including: temsirolimus (Torisel®); everolimus (Afinitor®; WO94/09010); ridaforolimus (also known as deforolimus or AP23573); rapalogs, e.g., as disclosed in WO98/02441 and WO01/14387, e.g. AP23464 and AP23841; 40-(2-hydroxyethyl)rapamycin; 40-[3-hydroxy(hydroxymethyl)methylpropanoate]-rapamycin (also known as CC1779); 40-epi-(tetrazolyt)-rapamycin (also called ABT578); 32-deoxorapamycin; 16-pentynyloxy-32(S)-dihydrorapanycin; derivatives disclosed in WO05/005434; derivatives disclosed in U.S. Pat. Nos. 5,258,389, 5,118,677, 5,118,678, 5,100,883, 5,151,413, 5,120,842, and 5,256,790, and in WO94/090101, WO92/05179, WO93/111130, WO94/02136, WO94/02485, WO95/14023, WO94/02136, WO95/16691, WO96/41807, WO96/41807, and WO2018204416; and phosphorus-containing rapamycin derivatives (e.g., WO05/016252). In some embodiments, the mTOR inhibitor is a bisteric inhibitor (see, e.g., WO2018204416, WO2019212990 and WO2019212991), such as RMC-5552, having the structure

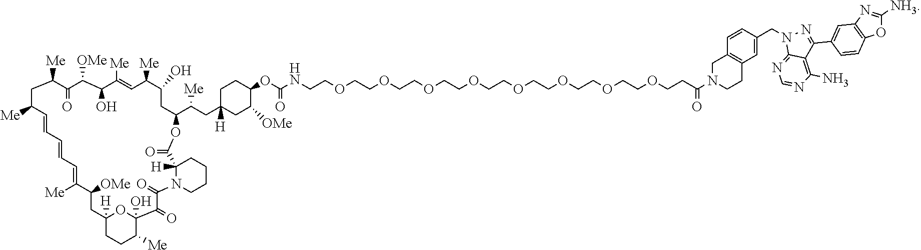

BRAF inhibitors that may be used in combination with compounds of the invention are known in the art and include, for example, vemurafenib, dabrafenib, and encorafenib. A BRAF may comprise a Class 3 BRAF mutation. In some embodiments, the Class 3 BRAF mutation is selected from one or more of the following amino acid substitutions in human BRAF: D287H; P367R; V459L; G466V; G466E; G466A; S467L; G469E; N581S; N581I; D594N; D594G; D594A; D594H; F595L; G596D; G596R and A762E.

MCL-1 inhibitors are known in the art and include, but are not limited to, AMG-176, MIK665, and S63845. The myeloid cell leukemia-1 (MCL-1) protein is one of the key anti-apoptotic members of the B-cell lymphoma-2 (BCL-2) protein family. Over-expression of MCL-1 has been closely related to tumor progression as well as to resistance, not only to traditional chemotherapies but also to targeted therapeutics including BCL-2 inhibitors such as ABT-263.

In some embodiments, the additional therapeutic agent is a SHP2 inhibitor. SHP2 inhibitors are known in the art. SHP2 is a non-receptor protein tyrosine phosphatase encoded by the PTPN11 gene that contributes to multiple cellular functions including proliferation, differentiation, cell cycle maintenance and migration. SHP2 has two N-terminal Src homology 2 domains (N—SH2 and C—SH2), a catalytic domain (PTP), and a C-terminal tail. The two SH2 domains control the subcellular localization and functional regulation of SHP2. The molecule exists in an inactive, self-inhibited conformation stabilized by a binding network involving residues from both the N—SH2 and PTP domains. Stimulation by, for example, cytokines or growth factors acting through receptor tyrosine kinases (RTKs) leads to exposure of the catalytic site resulting in enzymatic activation of SHP2.

SHP2 is involved in signaling through the RAS-mitogen-activated protein kinase (MAPK), the JAK-STAT or the phosphoinositol 3-kinase-AKT pathways. Mutations in the PTPN11 gene and subsequently in SHP2 have been identified in several human developmental diseases, such as Noonan Syndrome and Leopard Syndrome, as well as human cancers, such as juvenile myelomonocytic leukemia, neuroblastoma, melanoma, acute myeloid leukemia and cancers of the breast, lung, and colon. Some of these mutations destabilize the auto-inhibited conformation of SHP2 and promote autoactivation or enhanced growth factor driven activation of SHP2. SHP2, therefore, represents a highly attractive target for the development of novel therapies for the treatment of various diseases including cancer. A SHP2 inhibitor (e.g., RMC-4550 or SHP099) in combination with a RAS pathway inhibitor (e.g., a MEK inhibitor) have been shown to inhibit the proliferation of multiple cancer cell lines in vitro (e.g., pancreas, lung, ovarian and breast cancer). Thus, combination therapy involving a SHP2 inhibitor with a RAS pathway inhibitor could be a general strategy for preventing tumor resistance in a wide range of malignancies.

Non-limiting examples of such SHP2 inhibitors that are known in the art, include: Chen et al. Mol Pharmacol. 2006, 70, 562; Sarver et al., J. Med. Chem. 2017, 62, 1793; Xie et al., J. Med. Chem. 2017, 60, 113734; and Igbe et al., Oncotarget, 2017, 8, 113734; and PCT applications: WO 2023282702, WO 2023280283, WO 2023280237, WO 2023018155, WO 2023011513, WO 2022271966, WO 2022271964, WO 2022271911, WO 2022259157, WO 2022242767, WO 2022241975, WO 2022237676, WO 2022237367, WO 2022237178, WO 2022235822, WO 20222084008, WO 2022135568, WO 2021176072, WO 2021171261, WO 2021149817, WO 2021148010, WO 2021147879, WO 2021143823, WO 2021143701, WO 2021143680, WO 2021121397, WO 2021119525, WO 2021115286, WO 2021110796, WO 2021088945, WO 2021073439, WO 2021061706, WO 2021061515, WO 2021043077, WO 2021033153, WO 2021028362, WO 2021033153, WO 2021028362, WO 2021018287, WO 2020259679, WO 2020249079, WO 2020210384, WO 2020201991, WO 2020181283, WO 2020177653, WO 2020165734, WO 2020165733, WO 2020165732, WO 2020156243, WO 2020156242, WO 2020108590, WO 2020104635, WO 2020094104, WO 2020094018, WO 2020081848, WO 2020073949, WO 2020073945, WO 2020072656, WO 2020065453, WO 2020065452, WO 2020063760, WO 2020061103, WO 2020061101, WO 2020033828, WO 2020033286, WO 2020022323, WO 2019233810, WO 2019213318, WO 2019183367, WO 2019183364, WO 2019182960, WO 2019167000, WO 2019165073, WO 2019158019, WO 2019152454, WO 2019051469, WO 2019051084, WO 2018218133, WO 2018172984, WO 2018160731, WO 2018136265, WO 2018136264, WO 2018130928, WO 2018129402, WO 2018081091, WO 2018057884, WO 2018013597, WO 2017216706, WO 2017211303, WO 2017210134, WO 2017156397, WO 2017100279, WO 2017079723, WO 2017078499, WO 2016203406, WO 2016203405, WO 2016203404, WO 2016196591, WO 2016191328, WO 2015107495, WO 2015107494, WO 2015107493, WO 2014176488, WO 2014113584, CN 115677661, CN 115677660, CN 115611869, CN 115521305, CN 115490697, CN 115466273, CN 115394612, CN 115304613, CN 115304612, CN 115300513, CN 115197225, CN 114957162, CN 114920759, CN 114716448, CN 114671879, CN 114539223, CN 114524772, CN 114213417, CN 114195799, CN 114163457, CN 113896710, CN 113248521, CN 113248449, CN 113135924, CN 113024508, CN 112920131, CN 112823796, CN 112409334, CN 112402385, CN 112174935, 111848599, CN 111704611, CN 111393459, CN 111265529, CN 110143949, CN 108113848, U.S. Ser. No. 11/179,397, U.S. Ser. No. 11/044,675, U.S. Ser. No. 11/034,705, U.S. Ser. No. 11/033,547, U.S. Ser. No. 11/001,561, U.S. Ser. No. 10/988,466, U.S. Ser. No. 10/954,243, U.S. Ser. No. 10/934,302, or U.S. Ser. No. 10/858,359, or a pharmaceutically acceptable salt, solvate, isomer (e.g., stereoisomer), prodrug, or tautomer thereof, each of which is incorporated herein by reference.

In some embodiments, a SHP2 inhibitor binds in the active site. In some embodiments, a SHP2 inhibitor is a mixed-type irreversible inhibitor. In some embodiments, a SHP2 inhibitor binds an allosteric site e.g., a non-covalent allosteric inhibitor. In some embodiments, a SHP2 inhibitor is a covalent SHP2 inhibitor, such as an inhibitor that targets the cysteine residue (C333) that lies outside the phosphatase's active site. In some embodiments a SHP2 inhibitor is a reversible inhibitor. In some embodiments, a SHP2 inhibitor is an irreversible inhibitor. In some embodiments, the SHP2 inhibitor is SHP099. In some embodiments, the SHP2 inhibitor is TNO155, having the structure:

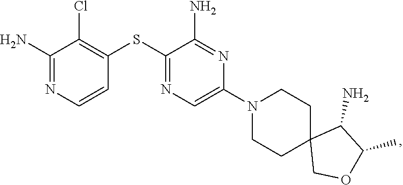 [SMILES: C[C@@H]1OCC2(CCN(c3cnc(Sc4ccnc(N)c4Cl)c(N)n3)CC2)[C@@H]1N]

or a pharmaceutically acceptable salt, solvate, isomer (e.g., stereoisomer), prodrug, or tautomer thereof. In some embodiments, the SHP2 inhibitor is RMC-4550. In some embodiments, the SHP2 inhibitor is RMC-4630, having the structure:

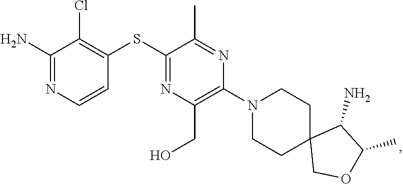 [SMILES: Cc1nc(N2CCC3(CC2)CO[C@@H](C)[C@H]3N)c(CO)nc1Sc1ccnc(N)c1Cl]

or a pharmaceutically acceptable salt, solvate, isomer (e.g., stereoisomer), prodrug, or tautomer thereof. In some embodiments, the SHP2 inhibitor is JAB-3068, having the structure

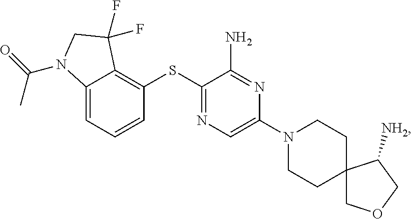 [SMILES: CC(=O)N1CC(F)(F)c2c(Sc3ncc(N4CCC5(CC4)COC[C@H]5N)nc3N)cccc21]

or a pharmaceutically acceptable salt, solvate, isomer (e.g., stereoisomer), prodrug, or tautomer thereof. In some embodiments, the SHP2 inhibitor is JAB-3312. In some embodiments, the SHP2 inhibitor is the following compound,

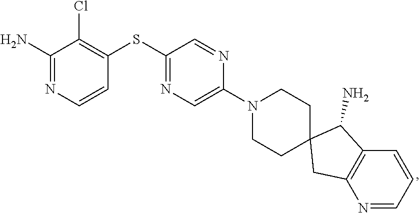 [SMILES: Nc1nccc(Sc2cnc(N3CCC4(CC3)Cc3ncccc3[C@H]4N)cn2)c1Cl]

or a pharmaceutically acceptable salt, solvate, isomer (e.g., stereoisomer), prodrug, or tautomer thereof. In some embodiments, the SHP2 inhibitor is RLY-1971, having the structure

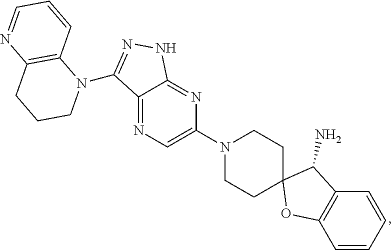 [SMILES: N[C@@H]1c2ccccc2OC12CCN(c1cnc3c(N4CCCc5ncccc54)n[nH]c3n1)CC2]

or a pharmaceutically acceptable salt, solvate, isomer (e.g., stereoisomer), prodrug, or tautomer thereof. In some embodiments, the SHP2 inhibitor is ERAS-601. In some embodiments, the SHP2 inhibitor is BBP-398.

In some embodiments, the additional therapeutic agent is selected from the group consisting of a MEK inhibitor, a HER2 inhibitor, a SHP2 inhibitor, a CDK4/6 inhibitor, an mTOR inhibitor, a SOS1 inhibitor, and a PD-L1 inhibitor. In some embodiments, the additional therapeutic agent is selected from the group consisting of a MEK inhibitor, a SHP2 inhibitor, and a PD-L1 inhibitor. See, e.g., Hallin et al., Cancer Discovery, DOI: 10.1158/2159-8290 (Oct. 28, 2019) and Canon et al., Nature, 575:217 (2019). In some embodiments, a Ras inhibitor of the present invention is used in combination with a MEK inhibitor and a SOS1 inhibitor. In some embodiments, a Ras inhibitor of the present invention is used in combination with a PD-L1 inhibitor and a SOS1 inhibitor. In some embodiments, a Ras inhibitor of the present invention is used in combination with a PD-L1 inhibitor and a SHP2 inhibitor. In some embodiments, a Ras inhibitor of the present invention is used in combination with a MEK inhibitor and a SHP2 inhibitor. In some embodiments, a Ras inhibitor of the present invention is used in combination with a SHP2 inhibitor and a Ras inhibitor that inhibits multiple Ras isoforms and/or mutants (e.g., RMC-6236). In some embodiments, the cancer is lung cancer, and the treatment comprises administration of a Ras inhibitor of the present invention in combination with a second or third therapeutic agent, such as a SHP2 inhibitor and a Ras inhibitor that inhibits multiple Ras isoforms and/or mutants. In some embodiments, the cancer is colorectal cancer, and the treatment comprises administration of a Ras inhibitor of the present invention in combination with a second or third therapeutic agent, such as a SHP2 inhibitor and a Ras inhibitor that inhibits multiple Ras isoforms and/or mutants. In some embodiments, a Ras inhibitor of the present invention is used in combination with an immunotherapy, optionally in combination with a chemotherapeutic agent.

Proteasome inhibitors are known in the art and include, but are not limited to, carfilzomib (Kyprolis®), bortezomib (Velcade®), and oprozomib.

Immune therapies include, but are not limited to, monoclonal antibodies, immunomodulatory imides (IMiDs), GITR agonists, genetically engineered T-cells (e.g., CAR-T cells), bispecific antibodies (e.g., BiTEs), and anti-PD-1, anti-PD-L1, anti-CTLA4, anti-LAGI, and anti-OX40 agents). Other immune therapies are known in the art.

Immunomodulatory agents (IMiDs) are a class of immunomodulatory drugs (drugs that adjust immune responses) containing an imide group. The IMiD class includes thalidomide and its analogues (lenalidomide, pomalidomide, and apremilast).

Exemplary anti-PD-1 antibodies and methods for their use are described by Goldberg et al., Blood 2007, 110(1):186-192; Thompson et al., Clin. Cancer Res. 2007, 13(6):1757-1761; and WO06/121168 A1), as well as described elsewhere herein.

FGFR inhibitors are known in the art, such as pemigatinib and erdafitinib, including FGFR2 inhibitors and FGFR4 inhibitors. See, e.g., Cancers (Basel), 2021 June; 13(12) 2968.

BET inhibitors are known in the art, such as romidepsin, panobinostat and belinostat. See, e.g., British J. Cancer 124:1478 (2021).

PRMT5i inhibitors are known in the art, such as PF-0693999, PJ-68 and MRTX1719. See, e.g., Biomed. Pharmacotherapy 144:112252 (2021).

MAT2A inhibitors are known in the art, such as AG-270 and IDE397. See, e.g., Exp Opin Ther Patents (2022) DOI: 10.1080/13543776.2022.2119127.

GITR agonists include, but are not limited to, GITR fusion proteins and anti-GITR antibodies (e.g., bivalent anti-GITR antibodies), such as, a GITR fusion protein described in U.S. Pat. Nos. 6,111,090, 8,586,023, WO2010/003118 and WO2011/090754; or an anti-GITR antibody described, e.g., in U.S. Pat. No. 7,025,962, EP 1947183, U.S. Pat. Nos. 7,812,135, 8,388,967, 8,591,886, 7,618,632, EP 1866339, and WO2011/028683, WO2013/039954, WO05/007190, WO07/133822, WO05/055808, WO99/40196, WO01/03720, WO99/20758, WO06/083289, WO05/115451, and WO2011/051726.

Another example of a therapeutic agent that may be used in combination with the compounds of the invention is an anti-angiogenic agent. Anti-angiogenic agents are known in the art and are inclusive of, but not limited to, in vitro synthetically prepared chemical compositions, antibodies, antigen binding regions, radionuclides, and combinations and conjugates thereof. An anti-angiogenic agent can be an agonist, antagonist, allosteric modulator, toxin or, more generally, may act to inhibit or stimulate its target (e.g., receptor or enzyme activation or inhibition), and thereby promote cell death or arrest cell growth. In some embodiments, the one or more additional therapies include an anti-angiogenic agent.

Anti-angiogenic agents can be MMP-2 (matrix-metalloproteinase 2) inhibitors, MMP-9 (matrix-metalloproteinase 9) inhibitors, and COX-II (cyclooxygenase 11) inhibitors. Non-limiting examples of anti-angiogenic agents include rapamycin, temsirolimus (CCI-779), everolimus (RAD001), sorafenib, sunitinib, and bevacizumab. Examples of useful COX-II inhibitors include alecoxib, valdecoxib, and rofecoxib. Examples of useful matrix metalloproteinase inhibitors are described in WO96/33172, WO96/27583, WO98/07697, WO98/03516, WO98/34918, WO98/34915, WO98/33768, WO98/30566, WO90/05719, WO99/52910, WO99/52889, WO99/29667, WO99007675, EP0606046, EP0780386, EP1786785, EP1181017, EP0818442, EP1004578, and US20090012085, and U.S. Pat. Nos. 5,863,949 and 5,861,510. Preferred MMP-2 and MMP-9 inhibitors are those that have little or no activity inhibiting MMP-1. More preferred, are those that selectively inhibit MMP-2 or AMP-9 relative to the other matrix-metalloproteinases (i.e., MAP-1, MMP-3, MMP-4, MMP-5, MMP-6, MMP-7, MMP-8, MMP-10, MMP-11, MMP-12, and MMP-13). Some specific examples of MMP inhibitors are AG-3340, RO 32-3555, and RS 13-0830.

Further exemplary anti-angiogenic agents include KDR (kinase domain receptor) inhibitory agents (e.g., antibodies and antigen binding regions that specifically bind to the kinase domain receptor), anti-VEGF agents (e.g., antibodies or antigen binding regions that specifically bind VEGF (e.g., bevacizumab), or soluble VEGF receptors or a ligand binding region thereof) such as VEGF-TRAP™, and anti-VEGF receptor agents (e.g., antibodies or antigen binding regions that specifically bind thereto), VEGF inhibitors, EGFR inhibitory agents (e.g., antibodies or antigen binding regions that specifically bind thereto) such as Vectibix® (panitumumab), erlotinib (Tarceva®), anti-Angl and anti-Ang2 agents (e.g., antibodies or antigen binding regions specifically binding thereto or to their receptors, e.g., Tie2/Tek), and anti-Tie2 kinase inhibitory agents (e.g., antibodies or antigen binding regions that specifically bind thereto). Other anti-angiogenic agents include Campath, IL-8, B-FGF, Tek antagonists (US2003/0162712; U.S. Pat. No. 6,413,932), anti-TWEAK agents (e.g., specifically binding antibodies or antigen binding regions, or soluble TWEAK receptor antagonists; see U.S. Pat. No. 6,727,225), ADAM distintegrin domain to antagonize the binding of integrin to its ligands (US 2002/0042368), specifically binding anti-eph receptor or anti-ephrin antibodies or antigen binding regions (U.S. Pat. Nos. 5,981,245; 5,728,813; 5,969,110; 6,596,852; 6,232,447; 6,057,124 and patent family members thereof), and anti-PDGF-BB antagonists (e.g., specifically binding antibodies or antigen binding regions) as well as antibodies or antigen binding regions specifically binding to PDGF-BB ligands, and PDGFR kinase inhibitory agents (e.g., antibodies or antigen binding regions that specifically bind thereto). Additional anti-angiogenic agents include: SD-7784 (Pfizer, USA); cilengitide (Merck KGaA, Germany, EPO 0770622); pegaptanib octasodium, (Gilead Sciences, USA); Alphastatin, (BioActa, UK); M-PGA, (Celgene, USA, U.S. Pat. No. 5,712,291); ilomastat, (Arriva, USA, U.S. Pat. No. 5,892,112); emaxanib, (Pfizer, USA, U.S. Pat. No. 5,792,783); vatalanib, (Novartis, Switzerland); 2-methoxyestradiol (EntreMed, USA); TLC ELL-12 (Elan, Ireland); anecortave acetate (Alcon, USA); alpha-D148 Mab (Amgen, USA); CEP-7055 (Cephalon, USA); anti-Vn Mab (Crucell, Netherlands), DACantiangiogenic (ConjuChem, Canada); Angiocidin (InKine Pharmaceutical, USA); KM-2550 (Kyowa Hakko, Japan); SU-0879 (Pfizer, USA); CGP-79787 (Novartis, Switzerland, EP 0970070); ARGENT technology (Ariad, USA); YIGSR-Stealth (Johnson & Johnson, USA); fibrinogen-E fragment (BioActa, UK); angiogenic inhibitor (Trigen, UK); TBC-1635 (Encysive Pharmaceuticals, USA); SC-236 (Pfizer, USA); ABT-567 (Abbott, USA); Metastatin (EntreMed, USA); maspin (Sosei, Japan); 2-methoxyestradiol (Oncology Sciences Corporation, USA); ER-68203-00 (IV AX, USA); BeneFin (Lane Labs, USA); Tz-93 (Tsumura, Japan); TAN-1120 (Takeda, Japan); FR-111142 (Fujisawa, Japan, JP 02233610); platelet factor 4 (RepliGen, USA, EP 407122); vascular endothelial growth factor antagonist (Borean, Denmark); bevacizumab (pINN) (Genentech, USA); angiogenic inhibitors (SUGEN, USA); XL 784 (Exelixis, USA); XL 647 (Exelixis, USA); MAb, alpha5beta3 integrin, second generation (Applied Molecular Evolution, USA and MedImmune, USA); enzastaurin hydrochloride (Lilly, USA); CEP 7055 (Cephalon, USA and Sanofi-Synthelabo, France); BC 1 (Genoa Institute of Cancer Research, Italy); rBPI 21 and BPI-derived antiangiogenic (XOMA, USA); PI 88 (Progen, Australia); cilengitide (Merck KGaA, German; Munich Technical University, Germany, Scripps Clinic and Research Foundation, USA); AVE 8062 (Ajinomoto, Japan); AS 1404 (Cancer Research Laboratory, New Zealand); SG 292, (Telios, USA); Endostatin (Boston Childrens Hospital, USA); ATN 161 (Attenuon, USA); 2-methoxyestradiol (Boston Childrens Hospital, USA); ZD 6474, (AstraZeneca, UK); ZD 6126, (Angiogene Pharmaceuticals, UK); PPI 2458, (Praecis, USA); AZD 9935, (AstraZeneca, UK); AZD 2171, (AstraZeneca, UK); vatalanib (pINN), (Novartis, Switzerland and Schering AG, Germany); tissue factor pathway inhibitors, (EntreMed, USA); pegaptanib (Pinn), (Gilead Sciences, USA); xanthorrhizol, (Yonsei University, South Korea); vaccine, gene-based, VEGF-2, (Scripps Clinic and Research Foundation, USA); SPV5.2, (Supratek, Canada); SDX 103, (University of California at San Diego, USA); PX 478, (ProIX, USA); METASTATIN, (EntreMed, USA); troponin I, (Harvard University, USA); SU 6668, (SUGEN, USA); OXI 4503, (OXiGENE, USA); o-guanidines, (Dimensional Pharmaceuticals, USA); motuporamine C, (British Columbia University, Canada); CDP 791, (Celltech Group, UK); atiprimod (pINN), (GlaxoSmithKline, UK); E 7820, (Eisai, Japan); CYC 381, (Harvard University, USA); AE 941, (Aeterna, Canada); vaccine, angiogenic, (EntreMed, USA); urokinase plasminogen activator inhibitor, (Dendreon, USA); oglufanide (pINN), (Melmotte, USA); HIF-lalfa inhibitors, (Xenova, UK); CEP 5214, (Cephalon, USA); BAY RES 2622, (Bayer, Germany); Angiocidin, (InKine, USA); A6, (Angstrom, USA); KR 31372, (Korea Research Institute of Chemical Technology, South Korea); GW 2286, (GlaxoSmithKline, UK); EHT 0101, (ExonHit, France); CP 868596, (Pfizer, USA); CP 564959, (OSI, USA); CP 547632, (Pfizer, USA); 786034, (GlaxoSmithKline, UK); KRN 633, (Kirin Brewery, Japan); drug delivery system, intraocular, 2-methoxyestradiol; anginex (Maastricht University, Netherlands, and Minnesota University, USA); ABT 510 (Abbott, USA); AAL 993 (Novartis, Switzerland); VEGI (ProteomTech, USA); tumor necrosis factor-alpha inhibitors; SU 11248 (Pfizer, USA and SUGEN USA); ABT 518, (Abbott, USA); YH16 (Yantai Rongchang, China); S-3APG (Boston Childrens Hospital, USA and EntreMed, USA); MAb, KDR (ImClone Systems, USA); MAb, alpha5 beta (Protein Design, USA); KDR kinase inhibitor (Celltech Group, UK, and Johnson & Johnson, USA); GFB 116 (South Florida University, USA and Yale University, USA); CS 706 (Sankyo, Japan); combretastatin A4 prodrug (Arizona State University, USA); chondroitinase AC (IBEX, Canada); BAY RES 2690 (Bayer, Germany); AGM 1470 (Harvard University, USA, Takeda, Japan, and TAP, USA); AG 13925 (Agouron, USA); Tetrathiomolybdate (University of Michigan, USA); GCS 100 (Wayne State University, USA) CV 247 (Ivy Medical, UK); CKD 732 (Chong Kun Dang, South Korea); irsogladine, (Nippon Shinyaku, Japan); RG 13577 (Aventis, France); WX 360 (Wilex, Germany); squalamine, (Genaera, USA); RPI 4610 (Sirna, USA); heparanase inhibitors (InSight, Israel); KL 3106 (Kolon, South Korea); Honokiol (Emory University, USA); ZK CDK (Schering AG, Germany); ZK Angio (Schering AG, Germany); ZK 229561 (Novartis, Switzerland, and Schering AG, Germany); XMP 300 (XOMA, USA); VGA 1102 (Taisho, Japan); VE-cadherin-2 antagonists(ImClone Systems, USA); Vasostatin (National Institutes of Health, USA); Flk-1 (ImClone Systems, USA); TZ 93 (Tsumura, Japan); TumStatin (Beth Israel Hospital, USA); truncated soluble FLT 1 (vascular endothelial growth factor receptor 1) (Merck & Co, USA); Tie-2 ligands (Regeneron, USA); and thrombospondin 1 inhibitor (Allegheny Health, Education and Research Foundation, USA).

Further examples of therapeutic agents that may be used in combination with compounds of the invention include agents (e.g., antibodies, antigen binding regions, or soluble receptors) that specifically bind and inhibit the activity of growth factors, such as antagonists of hepatocyte growth factor (HGF, also known as Scatter Factor), and antibodies or antigen binding regions that specifically bind its receptor, c-Met. Such agents are known in the art.

Another example of a therapeutic agent that may be used in combination with compounds of the invention is an autophagy inhibitor. Autophagy inhibitors are known in the art and include, but are not limited to chloroquine, 3-methyladenine, hydroxychloroquine (Plaquenil™), bafilomycin A1, 5-amino-4-imidazole carboxamide riboside (AICAR), okadaic acid, autophagy-suppressive algal toxins which inhibit protein phosphatases of type 2A or type 1, analogues of cAMP, and drugs which elevate cAMP levels such as adenosine, LY204002, N6-mercaptopurine riboside, and vinblastine. In addition, antisense or siRNA that inhibits expression of proteins including but not limited to ATG5 (which are implicated in autophagy), may also be used. In some embodiments, the one or more additional therapies include an autophagy inhibitor.

Another example of a therapeutic agent that may be used in combination with compounds of the invention is an anti-neoplastic agent, which are known in the art. In some embodiments, the one or more additional therapies include an anti-neoplastic agent. Non-limiting examples of anti-neoplastic agents include acemannan, aclarubicin, aldesleukin, alemtuzumab, alitretinoin, altretamine, amifostine, aminolevulinic acid, amrubicin, amsacrine, anagrelide, anastrozole, cancer, ancestim, arglabin, arsenic trioxide, BAM-002 (Novelos), bexarotene, bicalutamide, broxuridine, capecitabine, celmoleukin, cetrorelix, cladribine, clotrimazole, cytarabine ocfosfate, DA 3030 (Dong-A), daclizumab, denileukin diftitox, deslorelin, dexrazoxane, dilazep, docetaxel, docosanol, doxercalciferol, doxifluridine, doxorubicin, bromocriptine, carmustine, cytarabine, fluorouracil, HIT diclofenac, interferon alfa, daunorubicin, doxorubicin, tretinoin, edelfosine, edrecolomab, eflornithine, emitefur, epirubicin, epoetin beta, etoposide phosphate, exemestane, exisulind, fadrozole, filgrastim, finasteride, fludarabine phosphate, formestane, fotemustine, gallium nitrate, gemcitabine, gemtuzumab zogamicin, gimeracil/oteracil/tegafur combination, glycopine, goserelin, heptaplatin, human chorionic gonadotropin, human fetal alpha fetoprotein, ibandronic acid, idarubicin, (imiquimod, interferon alfa, interferon alfa, natural, interferon alfa-2, interferon alfa-2a, interferon alfa-2b, interferon alfa-NI, interferon alfa-n3, interferon alfacon-1, interferon alpha, natural, interferon beta, interferon beta-la, interferon beta-Ib, interferon gamma, natural interferon gamma-la, interferon gamma-Ib, interleukin-1 beta, iobenguane, irinotecan, irsogladine, lanreotide, LC 9018 (Yakult), leflunomide, lenograstim, lentinan sulfate, letrozole, leukocyte alpha interferon, leuprorelin, levamisole+fluorouracil, liarozole, lobaplatin, lonidamine, lovastatin, masoprocol, melarsoprol, metoclopramide, mifepristone, miltefosine, mirimostim, mismatched double stranded RNA, mitoguazone, mitolactol, mitoxantrone, molgramostim, nafarelin, naloxone+pentazocine, nartograstim, nedaplatin, nilutamide, noscapine, novel erythropoiesis stimulating protein, NSC 631570 octreotide, oprelvekin, osaterone, oxaliplatin, paclitaxel, pamidronic acid, pegaspargase, peginterferon alfa-2b, pentosan polysulfate sodium, pentostatin, picibanil, pirarubicin, rabbit antithymocyte polyclonal antibody, polyethylene glycol interferon alfa-2a, porfimer sodium, raloxifene, raltitrexed, rasburiembodiment, rhenium Re 186 etidronate, RII retinamide, rituximab, romurtide, samarium (153 Sm) lexidronam, sargramostim, sizofiran, sobuzoxane, sonermin, strontium-89 chloride, suramin, tasonermin, tazarotene, tegafur, temoporfin, temozolomide, teniposide, tetrachlorodecaoxide, thalidomide, thymalfasin, thyrotropin alfa, topotecan, toremifene, tositumomab-iodine 131, trastuzumab, treosulfan, tretinoin, trilostane, trimetrexate, triptorelin, tumor necrosis factor alpha, natural, ubenimex, bladder cancer vaccine, Maruyama vaccine, melanoma lysate vaccine, valrubicin, verteporfin, vinorelbine, virulizin, zinostatin stimalamer, or zoledronic acid; abarelix; AE 941 (Aeterna), ambamustine, antisense oligonucleotide, bcl-2 (Genta), APC 8015 (Dendreon), decitabine, dexaminoglutethimide, diaziquone, EL 532 (Elan), EM 800 (Endorecherche), eniluracil, etanidazole, fenretinide, filgrastim SD01 (Amgen), fulvestrant, galocitabine, gastrin 17 immunogen, HLA-B7 gene therapy (Vical), granulocyte macrophage colony stimulating factor, histamine dihydrochloride, ibritumomab tiuxetan, ilomastat, IM 862 (Cytran), interleukin-2, iproxifene, LDI 200 (Milkhaus), leridistim, lintuzumab, CA 125 MAb (Biomira), cancer MAb (Japan Pharmaceutical Development), HER-2 and Fc MAb (Medarex), idiotypic 105AD7 MAb (CRC Technology), idiotypic CEA MAb (Trilex), LYM-1-iodine 131 MAb (Techni clone), polymorphic epithelial mucin-yttrium 90 MAb (Antisoma), marimastat, menogaril, mitumomab, motexafin gadolinium, MX 6 (Galderma), nelarabine, nolatrexed, P 30 protein, pegvisomant, pemetrexed, porfiromycin, prinomastat, RL 0903 (Shire), rubitecan, satraplatin, sodium phenylacetate, sparfosic acid, SRL 172 (SR Pharma), SU 5416 (SUGEN), TA 077 (Tanabe), tetrathiomolybdate, thaliblastine, thrombopoietin, tin ethyl etiopurpurin, tirapazamine, cancer vaccine (Biomira), melanoma vaccine (New York University), melanoma vaccine (Sloan Kettering Institute), melanoma oncolysate vaccine (New York Medical College), viral melanoma cell lysates vaccine (Royal Newcastle Hospital), or valspodar.

Additional examples of therapeutic agents that may be used in combination with compounds of the invention include ipilimumab (Yervoy®); tremelimumab; galiximab; nivolumab, also known as BMS-936558 (Opdivo®); pembrolizumab (Keytruda®); avelumab (Bavencio®); AMP224; BMS-936559; MPDL3280A, also known as RG7446; MEDI-570; AMG557; MGA271; IMP321; BMS-663513; PF-05082566; CDX-1127; anti-OX40 (Providence Health Services); huMAbOX40L; atacicept; CP-870893; lucatumumab; dacetuzumab; muromonab-CD3; ipilumumab; MEDI4736 (Imfinzi®); MSB0010718C; AMP 224; adalimumab (Humira®); ado-trastuzumab emtansine (Kadcyla®); aflibercept (Eylea®); alemtuzumab (Campath®); basiliximab (Simulect®); belimumab (Benlysta®); basiliximab (Simulect®); belimumab (Benlysta®); brentuximab vedotin (Adcetris®); canakinumab (Ilaris®); certolizumab pegol (Cimzia®); daclizumab (Zenapax®); daratumumab (Darzalex®); denosumab (Prolia®); eculizumab (Soliris®); efalizumab (Raptiva®); gemtuzumab ozogamicin (Mylotarg®); golimumab (Simponi®); ibritumomab tiuxetan (Zevalin®); infliximab (Remicade®); motavizumab (Numax®); natalizumab (Tysabri®); obinutuzumab (Gazyva®); ofatumumab (Arzerra®); omalizumab (Xolair®); palivizumab (Synagis®); pertuzumab (Perjeta®); pertuzumab (Perjeta®); ranibizumab (Lucentis®); raxibacumab (Abthrax®); tocilizumab (Actemra®); tositumomab; tositumomab-i-131; tositumomab and tositumomab-i-131 (Bexxar®); ustekinumab (Stelara®); AMG 102; AMG 386; AMG 479; AMG 655; AMG 706; AMG 745; and AMG 951.

The compounds described herein can be used in combination with the agents disclosed herein or other suitable agents, depending on the condition being treated. Hence, in some embodiments the one or more compounds of the disclosure will be co-administered with other therapies as described herein. When used in combination therapy, the compounds described herein may be administered with the second agent simultaneously or separately. This administration in combination can include simultaneous administration of the two agents in the same dosage form, simultaneous administration in separate dosage forms, and separate administration. That is, a compound described herein and any of the agents described herein can be formulated together in the same dosage form and administered simultaneously. Alternatively, a compound of the invention and any of the therapies described herein can be simultaneously administered, wherein both the agents are present in separate formulations. In another alternative, a compound of the present disclosure can be administered and followed by any of the therapies described herein, or vice versa. In some embodiments of the separate administration protocol, a compound of the invention and any of the therapies described herein are administered a few minutes apart, or a few hours apart, or a few days apart.

In some embodiments of any of the methods described herein, the first therapy (e.g., a compound of the invention) and one or more additional therapies are administered simultaneously or sequentially, in either order. The first therapeutic agent may be administered immediately, up to 1 hour, up to 2 hours, up to 3 hours, up to 4 hours, up to 5 hours, up to 6 hours, up to 7 hours, up to, 8 hours, up to 9 hours, up to 10 hours, up to 11 hours, up to 12 hours, up to 13 hours, 14 hours, up to hours 16, up to 17 hours, up 18 hours, up to 19 hours up to 20 hours, up to 21 hours, up to 22 hours, up to 23 hours, up to 24 hours, or up to 1-7, 1-14, 1-21 or 1-30 days before or after the one or more additional therapies.

The invention also features kits including (a) a pharmaceutical composition including an agent (e.g., a compound of the invention) described herein, and (b) a package insert with instructions to perform any of the methods described herein. In some embodiments, the kit includes (a) a pharmaceutical composition including an agent (e.g., a compound of the invention) described herein, (b) one or more additional therapies (e.g., non-drug treatment or therapeutic agent), and (c) a package insert with instructions to perform any of the methods described herein.

As one aspect of the present invention contemplates the treatment of the disease or symptoms associated therewith with a combination of pharmaceutically active compounds that may be administered separately, the invention further relates to combining separate pharmaceutical compositions in kit form. The kit may comprise two separate pharmaceutical compositions: a compound of the present invention, and one or more additional therapies. The kit may comprise a container for containing the separate compositions such as a divided bottle or a divided foil packet. Additional examples of containers include syringes, boxes, and bags. In some embodiments, the kit may comprise directions for the use of the separate components. The kit form is particularly advantageous when the separate components are preferably administered in different dosage forms (e.g., oral and parenteral), are administered at different dosage intervals, or when titration of the individual components of the combination is desired by the prescribing health care professional.

### Examples

The disclosure is further illustrated by the following examples and synthesis examples, which are not to be construed as limiting this disclosure in scope or spirit to the specific procedures herein described. It is to be understood that the examples are provided to illustrate certain embodiments and that no limitation to the scope of the disclosure is intended thereby. It is to be further understood that resort may be had to various other embodiments, modifications, and equivalents thereof which may suggest themselves to those skilled in the art without departing from the spirit of the present disclosure or scope of the appended claims.

### Chemical Syntheses

Definitions used in the following examples and elsewhere herein are:
CH₂Cl₂, DCM Methylene chloride, Dichloromethane
CH₃CN, MeCN Acetonitrile
CuI Copper (I) iodide
DIPEA Diisopropylethyl amine
DMF N,N-Dimethylformamide
EtOAc Ethyl acetate
h hour
H₂O Water
HCl Hydrochloric acid
K₃PO₄ Potassium phosphate (tribasic)
MeOH Methanol
Na₂SO₄ Sodium sulfate
NMP N-methyl pyrrolidone
Pd(dppf)Cl₂ [1,1′-Bis(diphenylphosphino)ferrocene]dichloropalladium(II)

### Instrumentation

Mass spectrometry data collection took place with a Shimadzu LCMS-2020, an Agilent 1260LC-6120/6125MSD, a Shimadzu LCMS-2010EV, or a Waters Acquity UPLC, with either a QDa detector or SQ Detector 2. Samples were injected in their liquid phase onto a C-18 reverse phase. The compounds were eluted from the column using an acetonitrile gradient and fed into the mass analyzer. Initial data analysis took place with either Agilent ChemStation, Shimadzu LabSolutions, or Waters MassLynx. NMR data was collected with either a Bruker AVANCE III HD 400 MHz, a Bruker Ascend 500 MHz instrument, or a Varian 400 MHz, and the raw data was analyzed with either TopSpin or Mestrelab Mnova.

### Synthesis of Intermediates

### Intermediate 1. Synthesis of (2S)-2-{1-[(2S,3S)-1-(tert-butoxycarbonyl)-2-{[(4-methylbenzenesulfonyl)oxy]methyl}pyrrolidin-3-yl]-N-methylformamido}-3-methylbutanoic acid

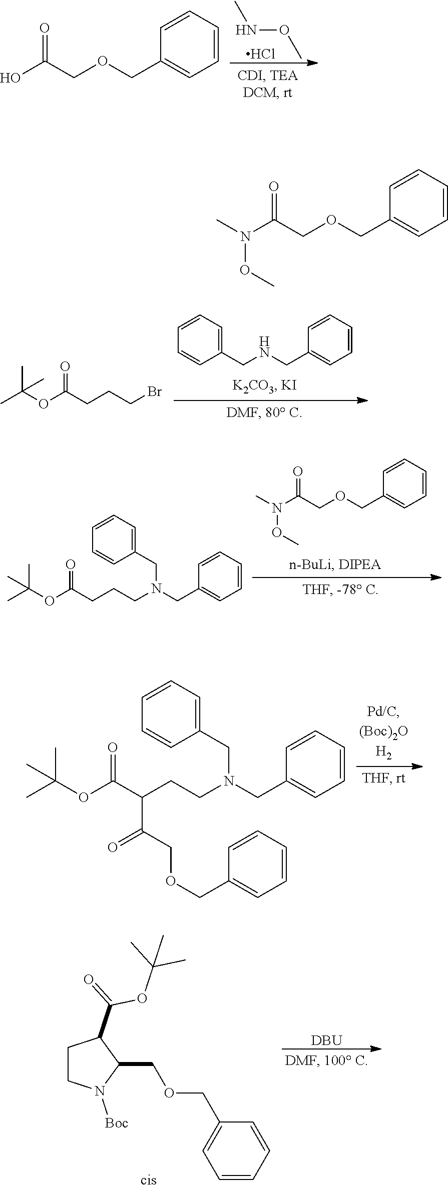

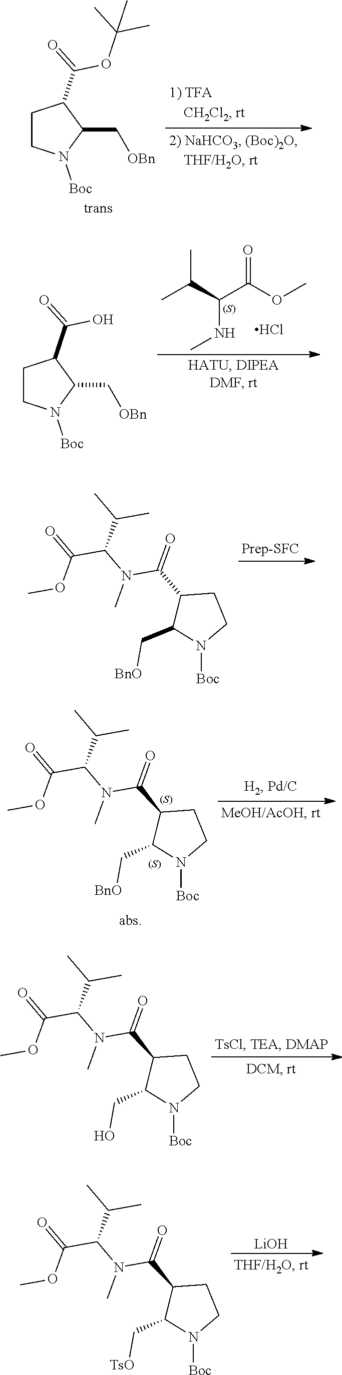

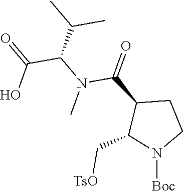 [SMILES: Cc1ccc(S(=O)(=O)OC[C@@H]2[C@@H](C(=O)N(C)[C@H](C(=O)O)C(C)C)CCN2C(=O)OC(C)(C)C)cc1]

Step 1. To a stirred solution of (benzyloxy)acetic acid (200 g, 1200 mmol) in DCM (2 L) was added CDI (254 g, 1560 mmol) at 0° C. The resulting mixture was stirred for 1 h at room temperature. TEA (335 mL, 2410 mmol) and N,O-dimethylhydroxylamine hydrochloride (164 g, 1690 mmol) were then added at 0° C. and the reaction mixture was stirred for 1 h at room temperature. The resulting mixture was treated with 1 M aq. HCl solution (4×800 mL) and concentrated under reduced pressure. The residue was purified by silica gel column chromatography to give 2-(benzyloxy)-N-methoxy-N-methylacetamide (238 g, 85% yield) as a light-yellow oil. LCMS (ESI) m/z: [M+H] calcd for C₁₁H₁₅NO₃: 210.1; found 210.1.

Step 2. To a solution of dibenzyl amine (177 g, 896 mmol) in DMF (500 mL) stirred at room temperature were added tert-butyl 4-bromobutanoate (200 g, 896 mmol), K₂CO₃ (248 g, 1800 mmol), and KI (14.9 g, 90.0 mmol). The resulting mixture was stirred for 2 h at 80° C. The reaction mixture was then quenched with H₂O at room temperature, extracted with EtOAc (3×500 mL), treated with brine, and concentrated under reduced pressure. The residue was purified by silica gel column chromatography to give tert-butyl 4-(dibenzylamino)butanoate (233 g, 71% yield) as a colorless oil. LCMS (ESI) m/z: [M+H] calcd for C₁₁H₁₅NO₃: 340.2; found 340.2.

Step 3. To a stirred solution of DIPA (116 mL, 821 mmol) in dry THE (1.5 L) was added n-BuLi (328 mL, 821 mmol) dropwise at −78° C. under an atmosphere of N₂. The resulting mixture was stirred for 30 min at 0° C. before being cooled back down to −78° C. tert-Butyl 4-(dibenzylamino)butanoate (186 g, 547 mmol) in dry THE (743 ml) was then added dropwise and the resulting mixture was stirred for 1 h at −78° C. 2-(benzyloxy)-N-methoxy-N-methylacetamide (137 g, 657 mmol) in dry THE (550 mL) was then added dropwise and the resulting mixture was stirred for 1 h at −78° C. The reaction was then quenched with sat. aq. NH₄Cl (500 mL), extracted with EtOAc (3×2 L), dried over anhydrous Na₂SO₄, filtered, and concentrated under reduced pressure. The residue was purified by silica gel column chromatography to give tert-butyl 4-(benzyloxy)-2-[2-(dibenzylamino)ethyl]-3-oxobutanoate (168 g, 57% yield) as a yellow oil. LCMS (ESI) m/z: [M+H] calcd for C₃₁H₃₇NO₄: 488.3; found 488.3.

Step 4. To a solution of tert-butyl 4-(benzyloxy)-2-[2-(dibenzylamino)ethyl]-3-oxobutanoate (168 g, 344 mmol) and Boc₂O (90.1 g, 413 mmol) in THE (1.7 L) was added 10% Pd/C (83.9 g, 788 mmol). The mixture was hydrogenated at room temperature under 2.5 atm H₂ for 18 h, filtered through a Celite pad, and concentrated under reduced pressure. The crude product (cis)-1,3-di-tert-butyl 2-[(benzyloxy)methyl]pyrrolidine-1,3-dicarboxylate (209 g, crude) was used in the next step directly without further purification. LCMS (ESI) m/z: [M+H] calcd for C₂₂H₃₃NO₅: 392.2; found 392.2.

Step 5. To a stirred mixture of (cis)-1,3-di-tert-butyl 2-[(benzyloxy)methyl]pyrrolidine-1,3-dicarboxylate (209 g, crude) in DMF (2 L) was added DBU (407 g, 2670 mmol) and the resulting mixture was stirred at 100° C. overnight. The reaction was quenched with H₂O at room temperature, extracted with EtOAc (3×1 L), treated with brine (3×500 mL), dried over anhydrous Na₂SO₄, filtered, and concentrated under reduced pressure. The residue was purified by silica gel column chromatography to give (trans)-1,3-di-tert-butyl 2-[(benzyloxy)methyl]pyrrolidine-1,3-dicarboxylate (109 g, 81% yield over 2 steps) as a light yellow oil. LCMS (ESI) m/z: [M+H] calcd for C₂₂H₃₃NO₅: 392.2; found 392.2.

Step 6. To a solution of (trans)-1,3-di-tert-butyl-2-[(benzyloxy)methyl]pyrrolidine-1,3-dicarboxylate (90 g, 230 mmol) in DCM (750 mL) was added TFA (250 mL) at 0° C. The reaction mixture was stirred overnight at room temperature. The mixture was then concentrated under reduced pressure and the residue was dissolved in a mixture of THE and H₂O. To this mixture was added NaHCO₃ (96.6 g, 1150 mmol) followed by Boc₂O (100 g, 460 mmol) at 0° C. The resulting mixture was stirred for 1 h at room temperature before being concentrated under reduced pressure. The resulting aqueous mixture was washed with hexane (2×200 mL), acidified to pH=6 with concentrated HCl, extracted with a 3:1 vol. mixture of DCM/i-PrOH (3×300 mL), dried over anhydrous Na₂SO₄, filtered, and concentrated under reduced pressure to afford (trans)-2-[(benzyloxy)methyl]-1-(tert-butoxycarbonyl)pyrrolidine-3-carboxylic acid (109 g, crude) as a light red oil. LCMS (ESI) m/z: [M+H] calcd for C₁₈H₂₅NO₅: 336.2; found 336.1.

Step 7. To a stirred mixture of (trans)-2-[(benzyloxy)methyl]-1-(tert-butoxycarbonyl)pyrrolidine-3-carboxylic acid (109 g, crude) and methyl (2S)-3-methyl-2-(methylamino)butanoate HCl salt (118 g, 650 mmol) in DMF (1 L) were added DIPEA (283 mL, 1630 mmol) and HATU (247 g, 650 mmol) at 0° C. The resulting mixture was stirred for 1 h at room temperature. The mixture was concentrated under reduced pressure and the residue was purified by silica gel column chromatography to give tert-butyl trans-2-[(benzyloxy)methyl]-3-{[(2S)-1-methoxy-3-methyl-1-oxobutan-2-yl](methyl)carbamoyl}pyrrolidine-1-carboxylate (74 g, 70% yield over 2 steps) as a red oil. LCMS (ESI) m/z: [M+H] calcd for C₂₅H₃₈N₂O₆: 463.3; found 463.0.

Step 8. tert-Butyl trans-2-[(benzyloxy)methyl]-3-{[(2S)-1-methoxy-3-methyl-1-oxobutan-2-yl](methyl)carbamoyl}pyrrolidine-1-carboxylate (30.0 g, 65.0 mmol) was purified by prep-SFC to give tert-butyl (2S,3S)-2-[(benzyloxy)methyl]-3-{[(2S)-1-methoxy-3-methyl-1-oxobutan-2-yl](methyl)carbamoyl}pyrrolidine-1-carboxylate (13.0 g, 43% yield) as a yellow oil. LCMS (ESI) m/z: [M+H−100] calcd for C₂₅H₃₈N₂O₆: 363.3; found 363.0.

Step 9. A mixture of tert-butyl (2S,3S)-2-[(benzyloxy)methyl]-3-{[(2S)-1-methoxy-3-methyl-1-oxobutan-2-yl](methyl)carbamoyl}pyrrolidine-1-carboxylate (12.0 g, 25.9 mmol) and 10% Pd/C (12.0 g, 113 mmol) in 9:1 vol. mixture of MeOH/AcOH (100 mL) was stirred overnight at room temperature under an atmosphere of H₂. The reaction mixture was concentrated under reduced pressure to remove the MeOH and the resulting mixture was basified to pH=8 with sat. aq. NaHCO₃then extracted with EtOAc. The combined organic extracts were washed with sat. aq. NaHCO₃, (3×100 mL) dried over anhydrous Na₂SO₄, filtered, and concentrated under reduced pressure to give tert-butyl (2S,3S)-2-(hydroxymethyl)-3-{[(2S)-1-methoxy-3-methyl-1-oxobutan-2-yl](methyl)carbamoyl}pyrrolidine-1-carboxylate (8.53 g, crude). LCMS (ESI) m/z: [M+H-Boc] calcd for C₁₈H₃₂N₂O₆: 272.2; found 272.9.

Step 10. To a stirred solution of tert-butyl (2S,3S)-2-(hydroxymethyl)-3-{[(2S)-1-methoxy-3-methyl-1-oxobutan-2-yl](methyl)carbamoyl}pyrrolidine-1-carboxylate (8.53 g, crude), TEA (19.0 mL, 137 mmol) and DMAP (279 mg, 2.28 mmol) in DCM was added a solution of TsCl (19.6 g, 103 mmol) in DCM dropwise at 0° C. The resulting mixture was stirred overnight at room temperature. The reaction mixture was quenched by the addition of H₂O (50 mL) at 0° C. and was then extracted with DCM (3×100 mL), dried over anhydrous Na₂SO₄, filtered, and concentrated under reduced pressure. The residue was purified by silica gel column chromatography to give tert-butyl (2S,3S)-3-{[(2S)-1-methoxy-3-methyl-1-oxobutan-2-yl](methyl)carbamoyl}-2-{[(4-methylbenzenesulfonyl)oxy]methyl}pyrrolidine-1-carboxylate (9.35 g, 69% yield over 2 steps) as a yellow solid. LCMS (ESI) m/z: [M+NH₄] calcd for C₂₅H₃₈N₂O₈: 544.3; found 544.0.

Step 11. To a stirred solution of tert-butyl (2S,3S)-3-{[(2S)-1-methoxy-3-methyl-1-oxobutan-2-yl](methyl)carbamoyl}-2-{[(4-methylbenzenesulfonyl)oxy]methyl}pyrrolidine-1-carboxylate (9.30 g, 17.7 mmol) in THE (75 mL) was added a solution of LiOH·H₂O (2.22 g, 53.0 mmol) in H₂O (15 mL) dropwise at 0° C. The resulting mixture was stirred overnight at room temperature. The reaction mixture was then concentrated under reduced pressure to remove the THF and the resulting mixture was diluted with H₂O (10 mL), acidified to pH=6 with aq. HCl, extracted with a 3:1 vol. mixture of DCM/i-PrOH (3×10 mL), dried over anhydrous Na₂SO₄, filtered, and concentrated under reduced pressure to give (2S)-2-{1-[(2S,3S)-1-(tert-butoxycarbonyl)-2-{[(4-methylbenzenesulfonyl)oxy]methyl}pyrrolidin-3-yl]-N-methylformamido}-3-methylbutanoic acid (9.62 g, crude), which was used in the next step directly without further purification. LCMS (ESI) m/z: [M+NH₄] calcd for C₂₄H₃₆N₂O₈: 530.3; found 530.3.

### Intermediate 1. Alternative Synthesis through Chiral Resolution

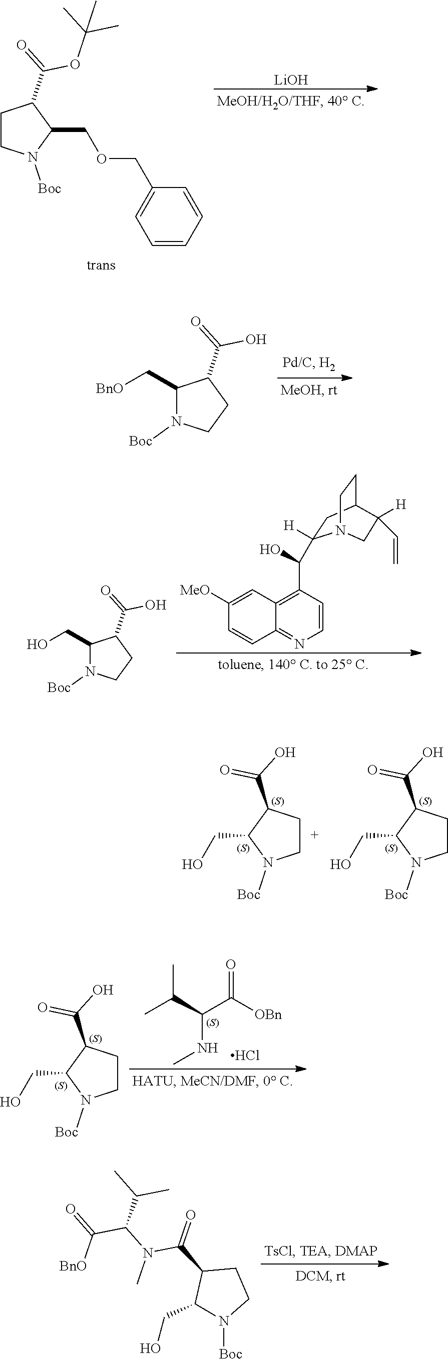

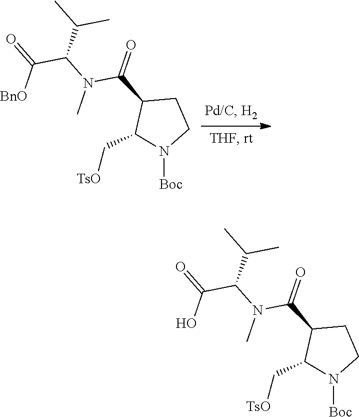 [SMILES: Cc1ccc(S(=O)(=O)OC[C@@H]2[C@@H](C(=O)N(C)[C@H](C(=O)OCc3ccccc3)C(C)C)CCN2C(=O)OC(C)(C)C)cc1 ; Cc1ccc(S(=O)(=O)OC[C@@H]2[C@@H](C(=O)N(C)[C@H](C(=O)O)C(C)C)CCN2C(=O)OC(C)(C)C)cc1]

Step 1. To a stirred solution of (cis)-1,3-di-tert-butyl 2-[(benzyloxy)methyl]pyrrolidine-1,3-dicarboxylate (130 g, 332 mmol) in THE (570 mL) were added MeOH (260 mL) and LiOH·H₂O (41.8 g, 996 mmol) in H₂O (260 mL) at room temperature under an atmosphere of N₂. The resulting mixture was stirred at 40° C. for 24 h. Four of these reactions were carried out in parallel on the same scale. These four reaction mixtures were combined and extracted with pet. ether (2×3 L), washed with 1 M aq. LiOH (2×2 L), acidified to pH=3 with 1 M aq. HCl, extracted with EtOAc (3×2 L), dried over anhydrous Na₂SO₄, filtered, and concentrated under reduced pressure to afford 2-[(benzyloxy)methyl]-1-(tert-butoxycarbonyl)pyrrolidine-3-carboxylic acid (414 g, 93% yield) as a white solid. LCMS (ESI) m/z: [M+H] calcd for C₁₈H₂₅NO₅: 336.2; found 336.2.

Step 2. To a stirred solution 2-[(benzyloxy)methyl]-1-(tert-butoxycarbonyl)pyrrolidine-3-carboxylic acid (207 g, 617 mmol) in MeOH (2 L) was added 10% wt. Pd/C (80.1 g) in portions at room temperature. The resulting mixture was stirred for 16 h at room temperature under an atmosphere of H₂ (10 atm). Two of these reactions were carried out in parallel on the same scale. The resulting two mixtures were combined then filtered. The filter cake was washed with MeOH (3×1 L), and the filtrate was concentrated under reduced pressure. The residue was purified by reversed phase flash column chromatography to give 1-(tert-butoxycarbonyl)-2-(hydroxymethyl)pyrrolidine-3-carboxylic acid (266 g, 88% yield) as a white solid. LCMS (ESI) m/z: [2M+Na] calcd for C₁₁H₁₉NO₅: 513.2; found 513.3.

Step 3. To a stirred mixture of 1-(tert-butoxycarbonyl)-2-(hydroxymethyl)pyrrolidine-3-carboxylic acid (26.6 g, 108 mmol) in toluene (1460 mL) was added (R)-[(2S,4R,5S)-5-ethenyl-1-azabicyclo[2.2.2]octan-2-yl](6-methoxyquinolin-4-yl)methanol (35.2 g, 108 mmol) in portions at room temperature under an atmosphere of N₂. The resulting mixture was stirred for 5 min. at room temperature. The mixture was then heated to 140° C. and was stirred under reflux for 1 min. and was then cooled to 25° C. and stirred for 14 h. Ten of these reactions were carried out in parallel on the same scale. The resulting ten mixtures were then combined and filtered. The filtrate was concentrated under reduced pressure, dissolved in THE (1 L), basified to pH=8 with 1N aq. NaOH, extracted with n-hexane (1×1L), and further extracted with EtOAc (3×500 mL). The aqueous mixture was acidified to pH=3 with 0.5M HCl and extracted with EtOAc (10×2 L). The combined organic extracts were dried over anhydrous Na₂SO₄, filtered, and concentrated under reduced pressure to give (2R,3R)-1-(tert-butoxycarbonyl)-2-(hydroxymethyl)pyrrolidine-3-carboxylic acid (120 g, >99% ee, 45% yield) as colorless oil. LCMS (ESI) m/z: [2M+Na] calcd for C₁₁H₁₉NO₅: 513.2; found 513.2.

The leftover filter cake was washed with i-PrOAc (130 mL) and was then diluted with ice water (300 mL). The resulting aqueous mixture was treated with 0.5 M aq. HCl (700 mL), basified to pH 8 with 1 N aq. NaOH (1000 mL), and extracted with i-PrOAc (3×700 mL). The remaining aqueous mixture was acidified to pH=3 with 0.5M aq. HCl, treated with NaCl (500 g), extracted with EtOAc (10×2 L), dried over anhydrous Na₂SO₄, filtered, and concentrated under reduced pressure to give (2S,3S)-1-(tert-butoxycarbonyl)-2-(hydroxymethyl)pyrrolidine-3-carboxylic acid (120 g, 95.1% ee, 45% yield) as colorless oil. LCMS (ESI) m/z: [2M+Na] calcd for C₁₁H₁₉NO₅: 513.2; found 513.2.

Step 4. To a stirred mixture of (2S,3S)-1-(tert-butoxycarbonyl)-2-(hydroxymethyl)pyrrolidine-3-carboxylic acid (35.0 g, 143 mmol) and benzyl (2S)-3-methyl-2-(methylamino)butanoate (56.8 g, 257 mmol) in MeCN (500 mL) were added 2,6-lutidine (153 g, 1430 mmol) and a solution of HATU (81.4 g, 214 mmol) in MeCN (200 mL) and DMF (70 mL) at −5° C. under an atmosphere of argon. The reaction mixture was stirred for 2 h at 0° C. before being quenched with H₂O (3 L). The resulting mixture was extracted with EtOAc (3×1 L), washed with brine (3×1 L), dried over anhydrous Na₂SO₄, filtered, and concentrated under reduced pressure. The residue was purified by reversed phase flash chromatography to give tert-butyl (2S,3S)-3-{[(2S)-1-(benzyloxy)-3-methyl-1-oxobutan-2-yl](methyl)carbamoyl}-2-(hydroxymethyl)pyrrolidine-1-carboxylate (42.0 g, 66% yield) as light yellow oil. LCMS (ESI) m/z: [M+Na] calcd for C₂₄H₃₆N₂O₆: 471.3; found 471.3.

Step 5. To a stirred solution of tert-butyl (2S,3S)-3-{[(2S)-1-(benzyloxy)-3-methyl-1-oxobutan-2-yl](methyl)carbamoyl}-2-(hydroxymethyl)pyrrolidine-1-carboxylate (73.0 g, 163 mmol) in DCM (600 mL) was added TEA (82.3 g, 814 mmol) followed by a solution of TsCl (40.3 g, 212 mmol) in DCM (100 mL) at −5° C. under an atmosphere of Argon. The reaction mixture was stirred for 16 h at 25° C. and was then quenched with H₂O (1 L). The mixture was acidified to pH=6 with 1 N aq. HCl, extracted with DCM (3×700 mL). treated with brine (3×500 mL), dried over anhydrous Na₂SO₄, filtered, and concentrated under reduced pressure. The residue was purified by silica gel column chromatography to give tert-butyl (2S,3S)-3-{[(2S)-1-(benzyloxy)-3-methyl-1-oxobutan-2-yl](methyl)carbamoyl}-2-{[(4-methylbenzenesulfonyl)oxy]methyl}pyrrolidine-1-carboxylate (81.0 g, 78% yield) as a light yellow solid. LCMS (ESI) m/z: [M+Na] calcd for C₃₁H₄₂N₂O₈S: 625.3; found 625.3.

Step 6. To a stirred solution of tert-butyl (2S,3S)-3-{[(2S)-1-(benzyloxy)-3-methyl-1-oxobutan-2-yl](methyl)carbamoyl}-2-{[(4-methylbenzenesulfonyl)oxy]methyl}pyrrolidine-1-carboxylate (87.0 g, 144 mmol) in THE (870 mL) was added 10% wt. Pd/C (40 g) at room temperature. The reaction mixture was stirred for 16 h at 25° C. under an atmosphere of H₂. The resulting mixture was then filtered, and the filter cake was washed with MeOH (3×500 mL). The filtrate was concentrated under reduced pressure to give (2S)-2-{1-[(2S,3S)-1-(tert-butoxycarbonyl)-2-{[(4-methylbenzenesulfony)oxy]methy}pyrrolidin-3-yl]-N-methylformamido}-3-methylbutanoic acid (71.0 g, 96% yield) as an off-white solid. LCMS (ESI) m/z: [2M+H] calcd for C₂₄H₃₆N₂O₈S: 1025.5; found 1025.3.

### Intermediate 1. Alternative Synthesis through Use of Chiral Pool Starting Material

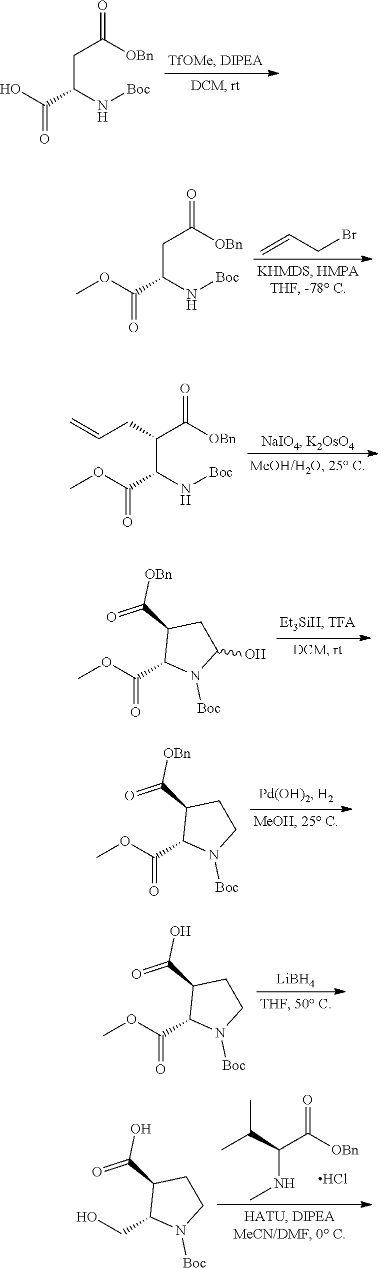

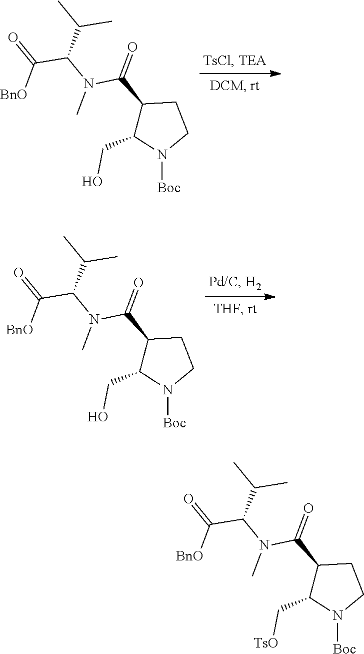 [SMILES: CC(C)[C@@H](C(=O)OCc1ccccc1)N(C)C(=O)[C@H]1CCN(C(=O)OC(C)(C)C)[C@@H]1CO ; CC(C)[C@@H](C(=O)OCc1ccccc1)N(C)C(=O)[C@H]1CCN(C(=O)OC(C)(C)C)[C@@H]1CO ; Cc1ccc(S(=O)(=O)OC[C@@H]2[C@@H](C(=O)N(C)[C@H](C(=O)OCc3ccccc3)C(C)C)CCN2C(=O)OC(C)(C)C)cc1]

Step 1. To a solution of N-(tert-butyloxycarbonyl)-L-aspartic acid β-benzyl ester (4.50 kg, 13.9 mol) in DCM (22.5 L) stirred at 0° C. under an atmosphere of N₂ were added DIPEA (2.16 kg, 16.7 mmol) followed by TfOMe (2.74 kg, 16.7 mmol). The resulting mixture was stirred for 1 h at 20° C. The reaction was quenched by the addition of 0.5M aq. HCl (11.2 L) and was then washed with H₂O (3×11.2 L). The organic phase was dried over anhydrous Na₂SO₄, filtered, and concentrated under reduced pressure. The resulting concentrated solution was treated with pet. ether, stirred at 20° C. for 30 min, and filtered. The filter cake was washed with pet. ether then was dried under reduced pressure to give 1-methyl 4-(phenylmethyl)-N-[(1,1-dimethylethoxy)carbonyl]-L-aspartate (4.76 kg, crude) as a white solid. LCMS (ESI) m/z: [M+Na] calcd for C₁₇H₂₃NO₆: 360.1; found 359.9.

Step 2. To a solution of 1-methyl 4-(phenylmethyl)-N-[(1,1-dimethylethoxy)carbonyl]-L-aspartate (1.50 kg, crude) in THF (12 L) stirred at −75° C. under an atmosphere of N₂ were added KHMDS (11.1 L, 1 M in THF) followed by HMPA (1.20 kg, 6.67 mol) then 3-bromoprop-1-ene (807 g, 6.67 mol). The reaction mixture was stirred at −75° C. for 2 h. The mixture was then diluted with EtOAc, treated with 30% aq. solution of citric acid, and stirred for 30 min. at 20° C. The aqueous layer was further extracted with EtOAc, treated with brine, dried over anhydrous Na₂SO₄, filtered, and concentrated under reduced pressure. The residue was purified by silica gel chromatography to give 1-methyl 4-(phenylmethyl)-(3S)—N-[(1,1-dimethylethoxy)carbonyl]-3-(2-propen-1-yl)-L-aspartate (908 g, 85.1% purity, 47% yield over 2 steps) as a brown oil. LCMS (ESI) m/z: [M+H] calcd for C₂₀H₂₇NO₆: 378.2; found 378.2.

Step 3. To a solution of 1-methyl 4-(phenylmethyl)-(3S)—N-[(1,1-dimethylethoxy)carbonyl]-3-(2-propen-1-yl)-L-aspartate (2.00 kg, 5.30 mol) in MeOH (20 L) and H₂O (8 L) stirred at 15° C. under an atmosphere of N₂ were added K₂OsO₄·2H₂O (19.5 g, 53.0 mmol) and NaIO₄ (3.40 kg, 15.9 mol). The reaction mixture was stirred at room temperature for 12 h and was then filtered. The filter cake was washed with MeOH, and the filtrate was quenched with 1M aq. HCl, treated with sat. aq. Na₂SO₃, and concentrated under reduced pressure to remove the MeOH. The resulting mixture was extracted with EtOAc, dried over anhydrous Na₂SO₄, filtered, and concentrated under reduced pressure to give 3-benzyl-1-(tert-butyl)-2-methyl-(2S,3S)-5-hydroxypyrrolidine-1,2,3-tricarboxylate (1.79 kg, crude) as a brown oil. LCMS (ESI) m/z: [2M+Na] calcd for C₁₉H₂₅NO₇: 781.3; found 781.4.

Step 4. To a solution of 3-benzyl-1-(tert-butyl)-2-methyl-(2S,3S)-5-hydroxypyrrolidine-1,2,3-tricarboxylate (2.00 kg, crude) in DCM (20 L) stirred at room temperature was added Et₃SiH (919 g, 7.91 mol) under an atmosphere of N₂. TFA (1.80 kg, 15.8 mol) was then added dropwise at 15° C. and the reaction mixture was stirred at room temperature for 3 h. The reaction was then quenched by the dropwise addition of sat. aq. NaHCO₃ at 15° C., and the resulting organic phase was washed with H₂O, dried over anhydrous Na₂SO₄, filtered, and concentrated under reduced pressure. The residue was then purified by silica gel chromatography to give 3-benzyl-1-(tert-butyl)-2-methyl-(2S,3S)-pyrrolidine-1,2,3-tricarboxylate (945 g, 69.4% purity, 44% yield over 2 steps) as a yellow oil. LCMS (ESI) m/z: [2M+Na] calcd for C₁₉H₂₅NO₆: 749.3; found 749.3.

Step 5. To a mixture of 20% Pd(OH)₂ (12.0 g) in MeOH (500 mL) was added a solution of 3-benzyl-1-(tert-butyl)-2-methyl-(2S,3S)-pyrrolidine-1,2,3-tricarboxylate (120 g, 330 mmol) in MeOH (500 mL) under an atmosphere of Argon. The reaction then placed under 45 psi H₂ and stirred at room temperature for 16 h. The mixture was then filtered through diatomaceous earth and the filter cake was rinsed with MeOH. The filtrate was concentrated under reduced pressure to give (2S,3S)-1-(tert-butoxycarbonyl)-2-(methoxycarbonyl)pyrrolidine-3-carboxylic acid (90 g, 78.0% purity, 78% yield) as a yellow oil. LCMS (ESI) m/z: [M-Boc+2H] calcd for C₁₂H₁₉NO₆: 174.1; found 174.1.

Step 6. To a solution of (2S,3S)-1-(tert-butoxycarbonyl)-2-(methoxycarbonyl)pyrrolidine-3-carboxylic acid (180 g, 659 mmol) in THE (1.8 L) stirred at 0° C. under an atmosphere of N₂ was added LiBH₄ (2 M in THF, 494 mL). The reaction mixture was stirred at 50° C. for 13 h. The mixture was then cooled to 0° C. and quenched with H₂O, adjusted to pH=4 by the addition of 1M aq. HCl, and diluted with brine. The aqueous layer was further extracted with EtOAc and the combined organic extracts were dried over anhydrous Na₂SO₄, filtered, and concentrated under reduced pressure to give (2S,3S)-1-(tert-butoxycarbonyl)-2-(hydroxymethyl)pyrrolidine-3-carboxylic acid (135 g, crude) as a white solid. This material was taken directly to the next step without further purification.

Step 7. To a solution of (2S,3S)-1-(tert-butoxycarbonyl)-2-(hydroxymethyl)pyrrolidine-3-carboxylic acid (285 g, 1.16 mol) in MeCN (2.3 L) and DMF (0.3 L) was added N-methyl-L-valine benzyl ester HCl salt (119 g, 0.465 mol). This mixture was cooled to 0° C. under an atmosphere of N₂ and 2,6-lutidine (622 g, 5.81 mol) was added followed by HATU (442 g, 1.16 mol). The reaction mixture was stirred for 2 h at 0° C. The reaction was quenched by the addition of brine and the organic extract was further treated with brine. The combined aqueous solutions were extracted with EtOAc, concentrated under reduced pressure, and purified by reversed-phase flash column chromatography to afford tert-butyl (2S,3S)-3-{[(2S)-1-methoxy-3-methyl-1-oxobutan-2-yl](methyl)carbamoyl}-2-(hydroxymethyl)pyrrolidine-1-carboxylate (120 g, 19% yield over 2 steps) as a white solid. LCMS (ESI) m/z: [M+Na] calcd for C₂₄H₃₆N₂O₆: 471.3; found 471.2.

Step 8. To a stirred solution of (2S,3S)-3-{[(2S)-1-methoxy-3-methyl-1-oxobutan-2-yl](methyl)carbamoyl}-2-(hydroxymethyl)pyrrolidine-1-carboxylate (168 g, 375 mmol) in DCM (1.7 L) were added TEA (190 g, 1870 mmol) followed by a solution of TsCl (92.8 g, 487 mmol) in DCM (1.7 L) dropwise at −5° C. under an atmosphere or Ar gas. The reaction mixture was stirred for 16 h at room temperature. The resulting mixture was then diluted with water, acidified to pH=6 with aq. 1 M HCl, extracted with DCM, treated with brine, dried over anhydrous Na₂SO₄, filtered, and concentrated under reduced pressure. The residue was purified by silica gel column chromatography, to give tert-butyl (2S,3S)-3-{[(2S)-1-(benzyloxy)-3-methyl-1-oxobutan-2-yl](methyl)carbamoyl}-2-{[(4-methylbenzenesulfonyl)oxy]methyl}pyrrolidine-1-carboxylate (216 g, 93% purity, 89% yield) as a light yellow solid. LCMS (ESI) m/z: [M+Na] calcd for C₃₁H₄₂N₂O₅S: 625.3; found 625.2.

Step 9. To a stirred solution of tert-butyl (2S,3S)-3-{[(2S)-1-(benzyloxy)-3-methyl-1-oxobutan-2-yl](methyl)carbamoyl}-2-{[(4-methylbenzenesulfonyl)oxy]methyl}pyrrolidine-1-carboxylate (216 g, 93% purity, 333 mmol) in THE (2 L) was added 10% wt. Pd/C (100 g) in portions at room temperature. The resulting mixture was stirred for 16 h at room temperature under an atmosphere of H₂ gas. The resulting mixture was filtered, the filter cake was washed with MeOH, and the filtrate was concentrated under reduced pressure to give (2S)-2-{1-[(2S,3S)-1-(tert-butoxycarbonyl)-2-{[(4-methylbenzenesulfonyl)oxy]methyl}pyrrolidin-3-yl]-N-methylformamido}-3-methylbutanoic acid (170 g, 91.6% purity, 91% yield) as an off-white solid. LCMS (ESI) m/z: [M+Na] calcd for C₃₁H₄₂N₂O₅S: 535.2; found 535.3.

### Intermediate 2. Synthesis of tert-butyl (2S,3S)-2-ethynyl-3-(methyl((S)-3-methyl-1-oxo-1-(2-(trimethylsilyl)ethoxy)butan-2-yl)carbamoyl)pyrrolidine-1-carboxylate

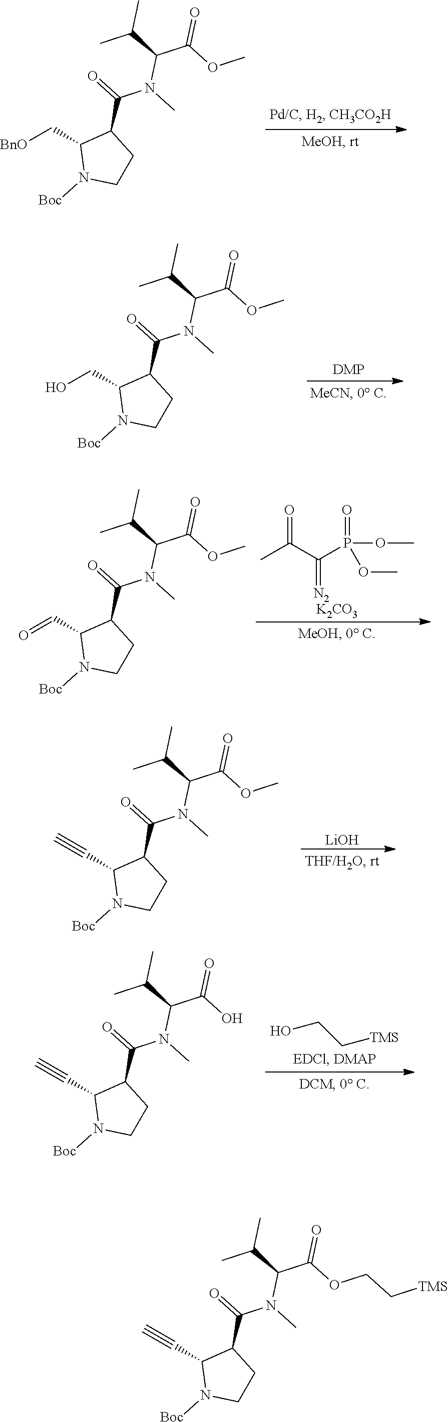

Step 1. To a solution of tert-butyl (2S,3S)-2-((benzyloxy)methyl)-3-(((S)-1-methoxy-3-methyl-1-oxobutan-2-yl)(methyl)carbamoyl)pyrrolidine-1-carboxylate (9.0 g, 19 mmol) in MeOH (81 mL) and acetic acid (9.0 mL) at room temperature was added Pd/C (9 g). The resulting mixture was stirred overnight under an atmosphere of H₂, filtered, and the filter cake washed with MeOH (3×100 mL). The filtrate was concentrated under reduced pressure to afford tert-butyl (2S,3S)-2-(hydroxymethyl)-3-(((S)-1-methoxy-3-methyl-1-oxobutan-2-yl)(methyl)carbamoyl)pyrrolidine-1-carboxylate (6.8 g, crude) which was taken directly to the next reaction without further purification. LCMS (ESI) m/z: [M+Na] calcd for C₁₈H₃₂N₂O₆: 395.2; found 395.2.

Step 2. To a solution of tert-butyl (2S,3S)-2-(hydroxymethyl)-3-(((S)-1-methoxy-3-methyl-1-oxobutan-2-yl)(methyl)carbamoyl)pyrrolidine-1-carboxylate (6.8 g, crude) in MeCN (70 mL) at 0° C. was added Dess-Martin periodinane (17.8 g, 41.9 mmol). The resulting mixture was stirred for 4 h at 0° C. and was then quenched by the addition of an aqueous solution Na₂S₂O₃. The aqueous mixture was extracted with DCM (3×100 mL) and the combined organic extracts were dried over Na₂SO₄, filtered, and concentrated under reduced pressure to afford tert-butyl (2S,3S)-2-formyl-3-(((S)-1-methoxy-3-methyl-1-oxobutan-2-yl)(methyl)carbamoyl)pyrrolidine-1-carboxylate (4.0 g, crude) which was used in the next reaction without further purification. LCMS (ESI) m/z: [M+H−100] calcd for C₁₈H₃₀N₂O₆: 271.2; found 271.1.

Step 3. To a solution of tert-butyl (2S,3S)-2-formyl-3-(((S)-1-methoxy-3-methyl-1-oxobutan-2-yl)(methyl)carbamoyl)pyrrolidine-1-carboxylate (3.8 g, crude) and K₂CO₃ (1.70 g, 12.3 mmol) in MeOH (40 mL) at 0° C. was added dimethyl (1-diazo-2-oxopropyl)phosphonate (1.97 g, 10.3 mmol). The resulting mixture was stirred for 3 h at 0° C. and was then quenched by the addition of H₂O. The aqueous mixture was extracted with EtOAc (3×50 mL), and the combined organic extracts were dried over Na₂SO₄, filtered, and concentrated under reduced pressure. Purification by reversed phase chromatography afforded tert-butyl (2S,3S)-2-ethynyl-3-(((S)-1-methoxy-3-methyl-1-oxobutan-2-yl)(methyl)carbamoyl)pyrrolidine-1-carboxylate (2.44 g, 33% yield over 3 steps) as a yellow solid. LCMS (ESI) m/z: [2M+NH₄] calcd for C₁₉H₃₀N₂O₅: 750.4; found 750.5.

Step 4. To a solution of tert-butyl (2S,3S)-2-ethynyl-3-(((S)-1-methoxy-3-methyl-1-oxobutan-2-yl)(methyl)carbamoyl)pyrrolidine-1-carboxylate (2.40 g, 6.55 mmol) in THE (12 mL) and H₂O (12 mL) at 0° C. was added LiOH·H₂O (550 mg, 13.1 mmol). The resulting mixture was stirred for 2 h at room temperature and was then acidified to pH=5 with 1 M aq. HCl. The aqueous mixture was extracted with EtOAc (3×20 mL), and the combined organic extracts were dried over Na₂SO₄, filtered, and concentrated under reduced pressure to afford N-((2S,3S)-1-(tert-butoxycarbonyl)-2-ethynylpyrrolidine-3-carbonyl)-N-methyl-L-valine (2.0 g, crude) which was used in the next reaction without further purification. LCMS (ESI) m/z: [M+H] calcd for C₁₈H₂₈N₂O₅: 353.2; found 353.1.

Step 5. To a solution of N-((2S,3S)-1-(tert-butoxycarbonyl)-2-ethynylpyrrolidine-3-carbonyl)-N-methyl-L-valine (1.98 g, crude) and 2-(trimethylsilyl)ethanol (1.33 g, 11.2 mmol) in DCM (20 mL) at 0° C. were added EDCI (2.15 g, 11.2 mmol) and DMAP (690 mg, 5.61 mmol). The resulting mixture was stirred for 1 h at room temperature and was then quenched at 0° C. by the addition of H₂O. The aqueous mixture was extracted with DCM (3×30 mL), and the combined organic extracts were dried over Na₂SO₄, filtered, and concentrated under reduced pressure. Purification by reversed phase chromatography afforded tert-butyl (2S,3S)-2-ethynyl-3-(methyl((S)-3-methyl-1-oxo-1-(2-(trimethylsilyl)ethoxy)butan-2-yl)carbamoyl)pyrrolidine-1-carboxylate (1.95 g, 45% yield over 2 steps) as a yellow oil. LCMS (ESI) m/z: [M+H] calcd for C₂₃H₄₀N₂O₅Si: 453.3; found 453.2.

### Intermediate 3. Synthesis of N-((2R,3S)-1-(tert-butoxycarbonyl)-2-vinylpyrrolidine-3-carbonyl)-N-methyl-L-valine

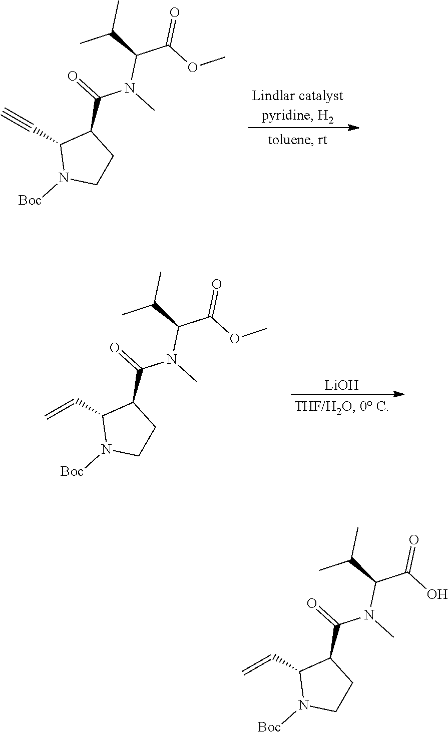 [SMILES: C#C[C@@H]1[C@@H](C(=O)N(C)[C@H](C(=O)OC)C(C)C)CCN1C(=O)OC(C)(C)C ; "Lindlar catalystpyridine, H2" ; C=C[C@@H]1[C@@H](C(=O)N(C)[C@H](C(=O)OC)C(C)C)CCN1C(=O)OC(C)(C)C ; "LiOH" ; C=C[C@@H]1[C@@H](C(=O)N(C)[C@H](C(=O)O)C(C)C)CCN1C(=O)OC(C)(C)C]

Step 1. To a solution of tert-butyl (2S,3S)-2-ethynyl-3-(((S)-1-methoxy-3-methyl-1-oxobutan-2-yl)(methyl)carbamoyl)pyrrolidine-1-carboxylate (7.0 g, 19.1 mmol) and pyridine (1.51 g, 19.1 mmol) in toluene (140 mL) was added Lindlar catalyst (7.0 g). The resulting mixture was stirred for 2 h at room temperature under an atmosphere of H₂, filtered, and the filter cake washed with MeOH (10×10 mL). The filtrate was concentrated under reduced pressure to afford tert-butyl (2R,3S)-3-(((S)-1-methoxy-3-methyl-1-oxobutan-2-yl)(methyl)carbamoyl)-2-vinylpyrrolidine-1-carboxylate (6.87 g, crude) which was used in the next reaction without further purification. LCMS (ESI) m/z: [M+NH₄] calcd for C₁₉H₃₂N₂O₅: 368.2; found 368.3.

Step 2. To a solution of tert-butyl (2R,3S)-3-(((S)-1-methoxy-3-methyl-1-oxobutan-2-yl)(methyl)carbamoyl)-2-vinylpyrrolidine-1-carboxylate (2.35 g, 6.38 mmol) in THE (30 mL) and H₂O (10 mL) was added LiOH·H₂O (540 mg, 12.8 mmol). The resulting mixture was stirred for 3 h at 0° C., concentrated under reduced pressure, and the concentrate acidified to pH=5 with 1M aq. HCl. The aqueous mixture was extracted with DCM (3×15 mL) and the combined organic extracts were dried over Na₂SO₄, filtered, and concentrated under reduced pressure to afford N-((2R,3S)-1-(tert-butoxycarbonyl)-2-vinylpyrrolidine-3-carbonyl)-N-methyl-L-valine (2.36 g, crude) which was used in subsequent reactions without further purification. LCMS (ESI) m/z: [M+Na] calcd for C₁₈H₃₀N₂O₅: 377.2; found 377.2.

### Intermediate 4. Synthesis of tert-butyl (1²S,1³S,6S,9S)-4⁵-iodo-9-isopropyl-6-((S)-3-(methoxycarbonyl)hexahydropyridazine-1-carbonyl)-10-methyl-8,11-dioxo-3-oxa-7,10-diaza-1(2,3)-pyrrolidina-4(1,3)-benzenacycloundecaphane-1¹-carboxylate

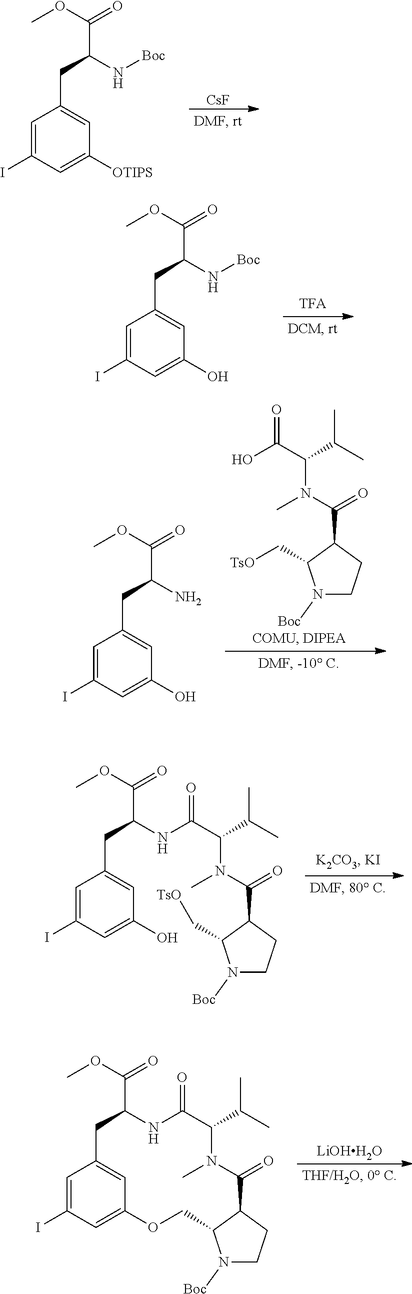

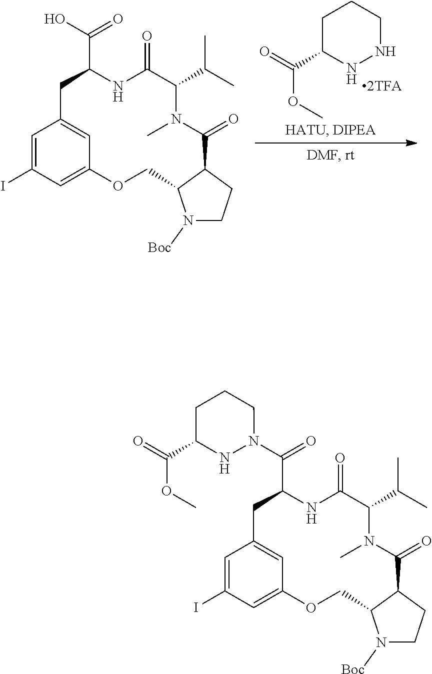

Step 1. To a solution of methyl (S)-2-((tert-butoxycarbonyl)amino)-3-(3-iodo-5-((triisopropylsilyl)oxy)phenyl)propanoate (5.0 g, 8.7 mmol) in DMF (50 mL) at 0° C. was added CsF (6.58 g, 43.3 mmol). The resulting mixture was stirred for 2 h at room temperature and quenched at 0° C. by the addition of H₂O (50 mL). The aqueous mixture was extracted with EtOAc (3×100 mL), and the combined organic extracts were washed with brine (3×100 mL), dried over Na₂SO₄, filtered, and concentrated under reduced pressure to afford methyl (S)-2-((tert-butoxycarbonyl)amino)-3-(3-hydroxy-5-iodophenyl)propanoate (5.02 g, crude), which was used without further purification. LCMS (ESI) m/z: [M+H] calcd for C₁₅H₂₀INO₅: 322.0; found 322.0.

Step 2. To a solution of methyl (S)-2-((tert-butoxycarbonyl)amino)-3-(3-hydroxy-5-iodophenyl)propanoate (4.97 g, crude) in DCM (50 mL) at 0° C. was added TFA (10 mL). The resulting mixture was stirred for 1 h at room temperature and basified to pH 8 by the addition of sat. aq. NaHCO₃. The aqueous layer was extracted with EtOAc (3×100 mL), and the combined organic extracts were dried over Na₂SO₄, filtered, and concentrated under reduced pressure to afford methyl (S)-2-amino-3-(3-hydroxy-5-iodophenyl)propanoate (4.01 g, crude), which was used without further purification. LCMS (ESI) m/z: [M+H] calcd for C₁₀H₁₂INO₃: 322.0; found 322.0.

Step 3. To a solution of methyl (S)-2-amino-3-(3-hydroxy-5-iodophenyl)propanoate (2.0 g, 6.2 mmol) in DMF (20 mL) at 0° C. were added DIPEA (43.4 mL, 249 mmol), N-((2S,3S)-1-(tert-butoxycarbonyl)-2-((tosyloxy)methyl)pyrrolidine-3-carbonyl)-N-methyl-L-valine (4.15 g, 8.10 mmol) and COMU (3.47 g, 8.10 mmol). The resulting mixture was stirred for 1 h at −10° C., quenched at −10° C. by the addition of H₂O (20 mL), and neutralized by the addition of 1 M aq. HCl. The aqueous mixture was extracted with EtOAc (3×50 mL), and the combined organic extracts were washed with brine (3×100 mL), dried over Na₂SO₄, filtered, and concentrated under reduced pressure. Purification by normal phase chromatography afforded tert-butyl (2S,3S)-3-(((S)-1-(((S)-3-(3-hydroxy-5-iodophenyl)-1-methoxy-1-oxopropan-2-yl)amino)-3-methyl-1-oxobutan-2-yl)(methyl)carbamoyl)-2-((tosyloxy)methyl)pyrrolidine-1-carboxylate (2.60 g, 74% yield over 3 steps) as a yellow solid. LCMS (ESI) m/z: [M+NH₄] calcd for C₃₄H₄₆IN₃O₁₀S: 833.2; found 833.2.

Step 4. To a solution of tert-butyl (2S,3S)-3-(((S)-1-(((S)-3-(3-hydroxy-5-iodophenyl)-1-methoxy-1-oxopropan-2-yl)amino)-3-methyl-1-oxobutan-2-yl)(methyl)carbamoyl)-2-((tosyloxy)methyl)pyrrolidine-1-carboxylate (1.83 g, 2.24 mmol) in DMF (183 mL) were added K₂CO₃ (3.10 g, 22.4 mmol) and KI (372 mg, 2.24 mmol). The resulting mixture was stirred for 2 h at 80° C. and quenched at 0° C. by the addition of H₂O (100 mL). The aqueous mixture was extracted with EtOAc (3×200 mL), and the combined organic extracts were washed with brine (3×400 mL), dried over Na₂SO₄, filtered, and concentrated under reduced pressure. Purification by normal phase chromatography afforded 1¹-(tert-butyl) 6-methyl (1²S,1³S,6S,9S)-4⁵-iodo-9-isopropyl-10-methyl-8,11-dioxo-3-oxa-7,10-diaza-1 (2,3)-pyrrolidina-4(1,3)-benzenacycloundecaphane-1¹,6-dicarboxylate (1.04 g, 66% yield) as a yellow solid. LCMS (ESI) m/z: [M+NH₄] calcd for C₂₇H₃₈IN₃O₇: 661.2; found 661.1.

Step 5. To a solution of 1¹-(tert-butyl) 6-methyl (1²S,1³S,6S,9S)-4⁵-iodo-9-isopropyl-10-methyl-8,11-dioxo-3-oxa-7,10-diaza-1(2,3)-pyrrolidina-4(1,3)-benzenacycloundecaphane-1¹,6-dicarboxylate (1.04 g, 1.61 mmol) in THE (6.0 mL) at 0° C. was added a solution of LiOH·H₂O (135 mg, 3.22 mmol) in H₂O (6.0 mL). The resulting mixture was stirred for 1.5 h at 0° C. and acidified to pH 6 by the addition of 1M aq. HCl. The aqueous mixture was extracted with EtOAc (3×50 mL), and the combined organic extracts were dried over Na₂SO₄, filtered, and concentrated under reduced pressure to afford (1²S,1³S,6S,9S)-1¹-(tert-butoxycarbonyl)-4⁵-iodo-9-isopropyl-10-methyl-8,11-dioxo-3-oxa-7,10-diaza-1(2,3)-pyrrolidina-4(1,3)-benzenacycloundecaphane-6-carboxylic acid (1.27 g, crude), which was used without further purification. LCMS (ESI) m/z: [M+NH₄] calcd for C₂₆H₃₆IN₃O₇: 647.2; found 647.1.

Step 6. To a solution of (1²S,1³S,6S,9S)-1¹-(tert-butoxycarbonyl)-4⁵-iodo-9-isopropyl-10-methyl-8,11-dioxo-3-oxa-7,10-diaza-1(2,3)-pyrrolidina-4(1,3)-benzenacycloundecaphane-6-carboxylic acid (1.15 g, crude) in DMF (15 mL) at 0° C. was added DIPEA (3.2 mL, 18.3 mmol), methyl (S)-hexahydropyridazine-3-carboxylate bis(trifluoroacetate) (1.36 g, 3.65 mmol) and HATU (1.39 g, 3.65 mmol). The resulting mixture was stirred for 1 h at room temperature, quenched at 0° C. by the addition of H₂O (20 mL), and acidified to pH 6 by the addition of 1M aq. HCl. The aqueous mixture was extracted with EtOAc (3×20 mL), and the combined organic extracts were washed with brine (3×30 mL), dried over Na₂SO₄, filtered, and concentrated under reduced pressure. Purification by normal phase chromatography afforded tert-butyl (1²S,1³S,6S,9S)-4⁵-iodo-9-isopropyl-6-((S)-3-(methoxycarbonyl)hexahydropyridazine-1-carbonyl)-10-methyl-8,11-dioxo-3-oxa-7,10-diaza-1(2,3)-pyrrolidina-4(1,3)-benzenacycloundecaphane-1¹-carboxylate (1.10 g, 72% yield over 2 steps) as a light yellow solid. LCMS (ESI) m/z: [M+NH₄] calcd for C₃₂H₄₆IN₅O₈: 773.3; found 773.3.

### Intermediate 5. Synthesis of tert-butyl ((6³S,4S)-1²-(5-(4-cyclopropylpiperazin-1-yl)-2-((S)-1-methoxyethyl)pyridin-3-yl)-1¹-ethyl-10,10-dimethyl-5,7-dioxo-2⁵-((triisopropylsilyl)oxy)-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)carbamate

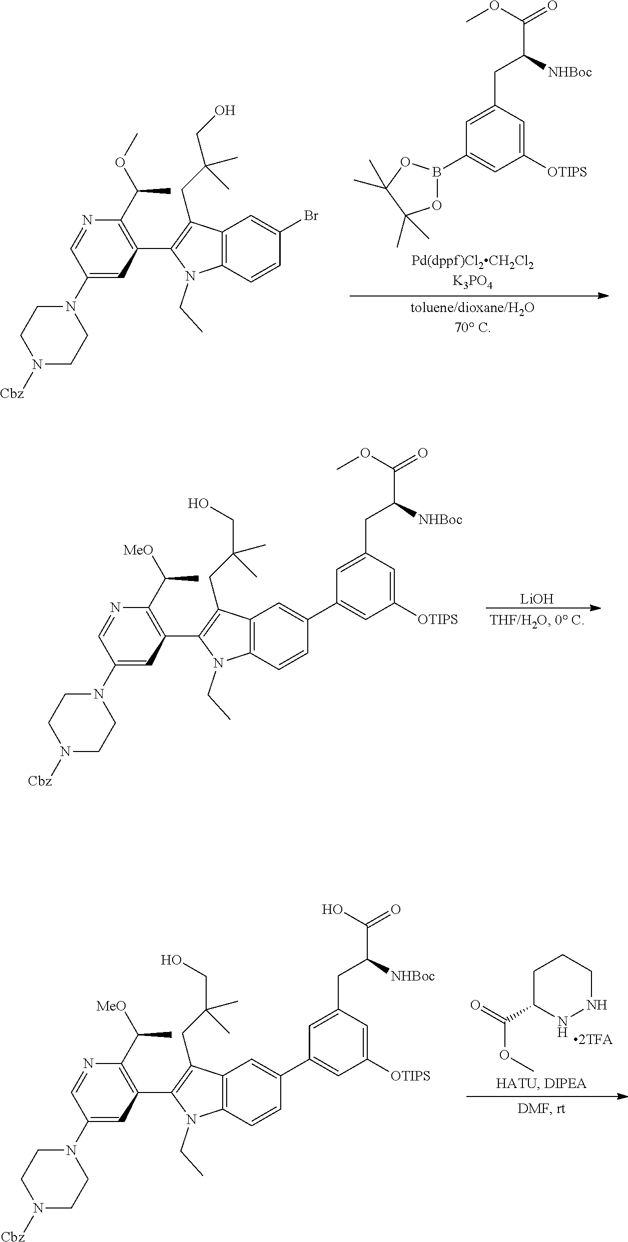

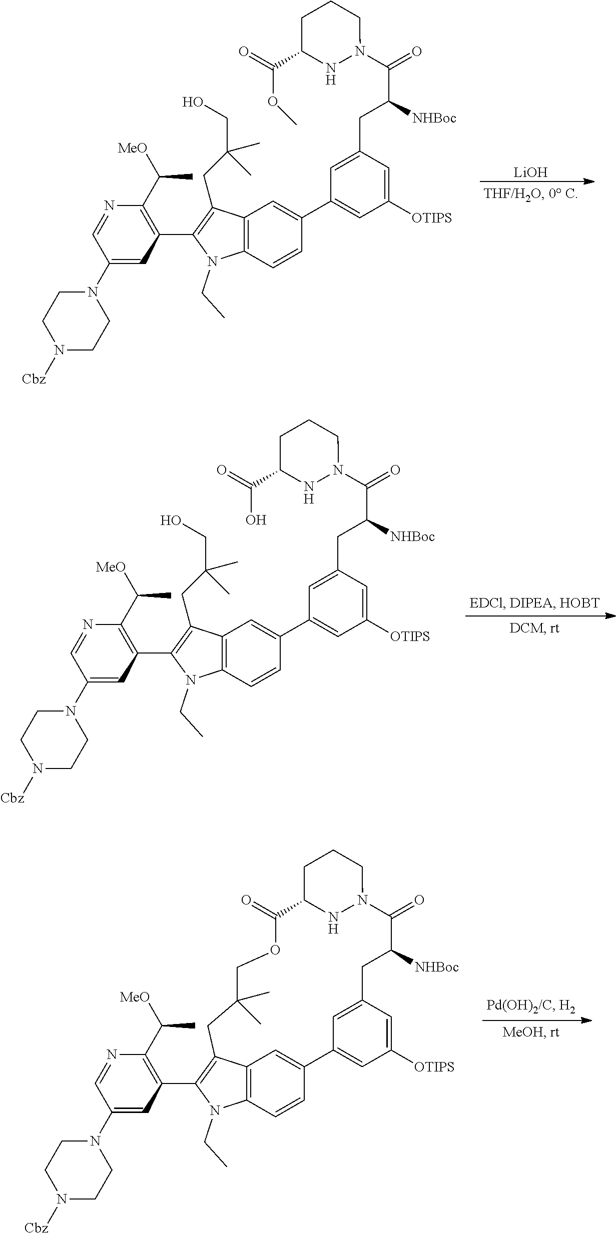

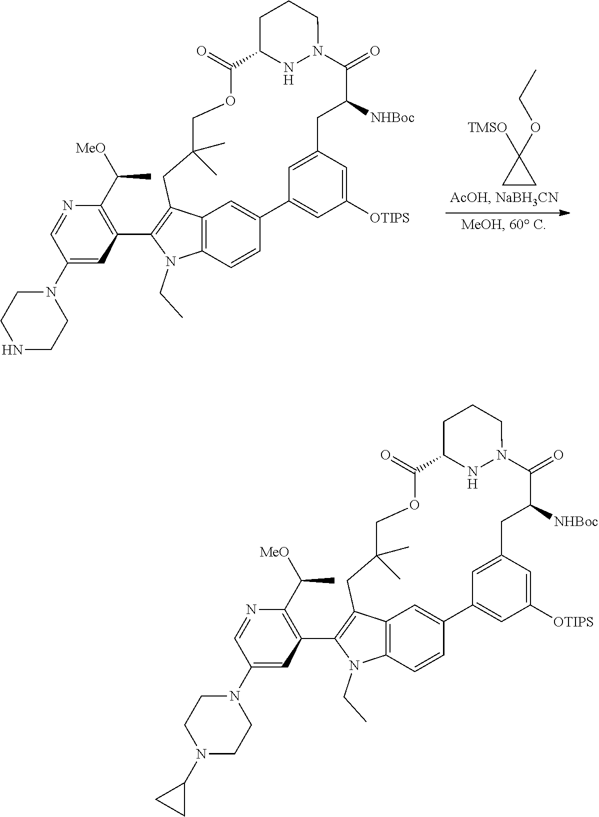

Step 1. To a solution of benzyl (S)-4-(5-(5-bromo-1-ethyl-3-(3-hydroxy-2,2-dimethylpropyl)-1H-indol-2-yl)-6-(1-methoxyethyl)pyridin-3-yl)piperazine-1-carboxylate (60.0 g, 90.4 mmol) and methyl (S)-2-((tert-butoxycarbonyl)amino)-3-(3-(4,4,5,5-tetramethyl-1,3,2-dioxaborolan-2-yl)-5-((triisopropylsilyl)oxy)phenyl)propanoate (78.3 g, 136 mmol) in toluene (600 mL) were added K₃PO₄ (57.6 g, 271 mmol) and Pd(dppf)Cl₂·CH₂Cl₂ (7.36 g, 9.04 mmol). The mixture was stirred overnight at 70° C. under an atmosphere of nitrogen atmosphere then was extracted with EtOAc (3×200 mL). The organic extracts were dried over anhydrous Na₂SO₄, filtered, and concentrated under reduced pressure. The residue was purified by silica gel column chromatography to afford benzyl 4-(5-(5-(3-((S)-2-((tert-butoxycarbonyl)amino)-3-methoxy-3-oxopropyl)-5-((triisopropylsilyl)oxy)phenyl)-1-ethyl-3-(3-hydroxy-2,2-dimethylpropyl)-1H-indol-2-yl)-6-((S)-1-methoxyethyl)pyridin-3-yl)piperazine-1-carboxylate (92.8 g, 90%) as a yellow solid. LCMS (ESI) m/z: [M+H] calcd for C₅₉H₈₃N₅O₉Si: 1034.6; found 1034.7.

Step 2. A solution of benzyl 4-(5-(5-(3-((S)-2-((tert-butoxycarbonyl)amino)-3-methoxy-3-oxopropyl)-5-((triisopropylsilyl)oxy)phenyl)-1-ethyl-3-(3-hydroxy-2,2-dimethylpropyl)-1H-indol-2-yl)-6-((S)-1-methoxyethyl)pyridin-3-yl)piperazine-1-carboxylate (92.8 g, 89.7 mmol) and LiOH (3.87 g, 161 mmol) in H₂O (1000 mL) and THE (1000 mL) was stirred overnight at 0° C. The resulting mixture was concentrated under reduced pressure and acidified to pH=5 with 1 M HCl (aq.). The resulting mixture was extracted with EtOAc (3×1 L), dried over anhydrous Na₂SO₄, filtered, and concentrated under reduced pressure to give (2S)-3-{3-[(2M)-2-(5-{4-[(benzyloxy)carbonyl]piperazin-1-yl}-2-[(1 S)-1-methoxyethyl]pyridin-3-yl)-1-ethyl-3-(3-hydroxy-2,2-dimethylpropyl)indol-5-yl]-5-[(triisopropylsilyl)oxy]phenyl}-2-[(tert-butoxycarbonyl)amino]propanoic acid (92.8 g, crude) as a yellow solid. LCMS (ESI) m/z: [M+H] calcd for C₅₈H₈₁N₅O₉Si: 1020.6; found 1020.7.

Step 3. To a stirred solution of (2S)-3-{3-[(2M)-2-(5-{4-[(benzyloxy)carbonyl]piperazin-1-yl}-2-[(1S)-1-methoxyethyl]pyridin-3-yl)-1-ethyl-3-(3-hydroxy-2,2-dimethylpropyl)indol-5-yl]-5-[(triisopropylsilyl)oxy]phenyl}-2-[(tert-butoxycarbonyl)amino]propanoic acid (91.8 g, 90.0 mmol), DIPEA (116 g, 900 mmol) and methyl (S)-hexahydropyridazine-3-carboxylate (19.5 g, 135 mmol) in DMF (1000 mL) was added HATU (68.4 g, 180 mmol) in portions at 0° C. The resulting mixture was stirred for 2 h at room temperature and was then diluted with deionized H₂O (2 L). The resulting mixture was extracted with EtOAc (3×2 L), washed with brine (3×4 L), dried over anhydrous Na₂SO₄, filtered, and concentrated under reduced pressure. The residue was purified by silica gel column chromatography to afford methyl (S)-1-((S)-3-(3-(2-(5-(4-((benzyloxy)carbonyl)piperazin-1-yl)-2-((S)-1-methoxyethyl)pyridin-3-yl)-1-ethyl-3-(3-hydroxy-2,2-dimethylpropyl)-1H-indol-5-yl)-5-((triisopropylsilyl)oxy)phenyl)-2-((tert-butoxycarbonyl)amino)propanoyl)hexahydropyridazine-3-carboxylate (94.6 g, 92%) as a yellow solid. LCMS (ESI) m/z: [M+H] calcd for C₆₄H₉₁N₇O₁₀Si: 1146.7; found 1146.9.

Step 4. A solution of (S)-1-((S)-3-(3-(2-(5-(4-((benzyloxy)carbonyl)piperazin-1-yl)-2-((S)-1-methoxyethyl)pyridin-3-yl)-1-ethyl-3-(3-hydroxy-2,2-dimethylpropyl)-1H-indol-5-yl)-5-((triisopropylsilyl)oxy)phenyl)-2-((tert-butoxycarbonyl)amino)propanoyl)hexahydropyridazine-3-carboxylate (110 g, 95.9 mmol) and LiOH (2.76 g, 115 mmol) in THE (900 mL) and H₂O (300 mL) was stirred for overnight at 0° C. The resulting mixture was concentrated under reduced pressure, acidified to pH=5 with 1M HCl (aq.), and extracted with EtOAc (3×500 mL). The combined organic extracts were dried over anhydrous Na₂SO₄, filtered, and concentrated under reduced pressure to give (S)-1-((S)-3-(3-(2-(5-(4-((benzyloxy)carbonyl)piperazin-1-yl)-2-((S)-1-methoxyethyl)pyridin-3-yl)-1-ethyl-3-(3-hydroxy-2,2-dimethylpropyl)-1H-indol-5-yl)-5-((triisopropylsilyl)oxy)phenyl)-2-((tert-butoxycarbonyl)amino)propanoyl)hexahydropyridazine-3-carboxylic acid (106 g, crude) as yellow solid. LCMS (ESI) m/z: [M+H] calcd for C₆₃H₈₉N₇O₁₀Si: 1132.6; found 1132.7.

Step 5. To a stirred solution of (S)-1-((S)-3-(3-(2-(5-(4-((benzyloxy)carbonyl)piperazin-1-yl)-2-((S)-1-methoxyethyl)pyridin-3-yl)-1-ethyl-3-(3-hydroxy-2,2-dimethylpropyl)-1H-indol-5-yl)-5-((triisopropylsilyl)oxy)phenyl)-2-((tert-butoxycarbonyl)amino)propanoyl)hexahydropyridazine-3-carboxylic acid (106 g, 93.2 mmol) and DIPEA (482 g, 3730 mmol) in DCM (10 L) were added HOBT (126 g, 932 mmol) and EDCI (536 g, 2800 mmol) in portions at 0° C. The resulting mixture was stirred for overnight at room temperature then was concentrated under reduced pressure and diluted with DCM (3 L). The resulting solution was washed with brine, treated with 1 M aq. HCl (2×4 L), neutralized with sat. aq. NaHCO₃ (4 L), dried over anhydrous Na₂SO₄, filtered, and concentrated under reduced pressure. The residue was purified by silica gel column chromatography to afford benzyl 4-(5-((6³S,4S)-4-((tert-butoxycarbonyl)amino)-1¹-ethyl-10,10-dimethyl-5,7-dioxo-2⁵-((triisopropylsilyl)oxy)-6¹,6²,6³,6⁴,6⁵,6⁶-hexahydro-1¹H-8-oxa-1(5,3)-indola-6(1,3)-pyridazina-2(1,3)-benzenacycloundecaphane-1²-yl)-6-((S)-1-methoxyethyl)pyridin-3-yl)piperazine-1-carboxylate (68.9 g, 63%) as yellow solid. LCMS (ESI) m/z: [M+H] calcd for C₆₃H₈₇N₇O₉Si: 1114.6; found 1115.0.

Step 6. A solution of benzyl 4-(5-((6³S,4S)-4-((tert-butoxycarbonyl)amino)-1¹-ethyl-10,10-dimethyl-5,7-dioxo-2⁵-((triisopropylsilyl)oxy)-6¹,6²,6³,6⁴,6⁵,6⁶-hexahydro-1 ¹H-8-oxa-1(5,3)-indola-6(1,3)-pyridazina-2(1,3)-benzenacycloundecaphane-1²-yl)-6-((S)-1-methoxyethyl)pyridin-3-yl)piperazine-1-carboxylate (58.7 g, 52.7 mmol) and Pd(OH)₂/C (30.0 g, 214 mmol) in MeOH (500 mL) was stirred for 3 h at room temperature under an atmosphere of H₂. The resulting mixture was filtered, and the filter cake was washed with MeOH (3×200 mL). The filtrate was then concentrated under reduced pressure to give tert-butyl ((6³S,4S)-1¹-ethyl-1²-(2-((S)-1-methoxyethyl)-5-(piperazin-1-yl)pyridin-3-yl)-10,10-dimethyl-5,7-dioxo-2⁵-((triisopropylsilyl)oxy)-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)carbamate (61.4 g, crude) as a white solid. LCMS (ESI) m/z: [M+H] calcd for C₅₅H₈₁N₇O₇Si: 980.6; found 980.7.

Step 7. To a solution of tert-butyl ((6³S,4S)-1¹-ethyl-1²-(2-((S)-1-methoxyethyl)-5-(piperazin-1-yl)pyridin-3-yl)-10,10-dimethyl-5,7-dioxo-2⁵-((triisopropylsilyl)oxy)-6¹,6²,6³,6⁴,6⁵,6⁶-hexahydro-1 ¹H-8-oxa-1(5,3)-indola-6(1,3)-pyridazina-2(1,3)-benzenacycloundecaphane-4-yl)carbamate (25.2 g, 25.7 mmol), (1-ethoxycyclopropoxy)trimethylsilane (11.4 g, 65.5 mmol), and AcOH (3.09 g, 51.4 mmol) in MeOH (250 mL) was added NaBH₃CN (3.23 g, 51.4 mmol) at 0° C. The mixture was stirred overnight at 60° C. under an atmosphere of N₂, basified to pH=8 with sat. aq. NaHCO₃ and concentrated under reduced pressure. The resulting residue was extracted with EtOAc (3×150 mL) and the combined organic extracts were dried over anhydrous Na₂SO₄, filtered, and concentrated under reduced pressure, and purified by silica gel column chromatography to afford tert-butyl ((6³S,4S)-1²-(5-(4-cyclopropylpiperazin-1-yl)-2-((S)-1-methoxyethyl)pyridin-3-yl)-1¹-ethyl-10,10-dimethyl-5,7-dioxo-2⁵-((triisopropylsily)oxy)-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)carbamate (19.3 g, 74% yield) as white solid. LCMS (ESI) m/z: [M+H] calcd for C₅₈H₈₅N₇O₇Si: 1020.6; found 1021.3.

### Intermediate 6. Synthesis of tert-butyl (3aS,6S,9S,15S,32aS)-21-(5-(4-cyclopropylpiperazin-1-yl)-2-((S)-1-methoxyethyl)pyridin-3-yl)-22-ethyl-6-isopropyl-5,19,19-trimethyl-4,7,10,16-tetraoxo-2,3,3a,4,5,6,7,8,9,10,13,14,15,16,18,19,20,22,32,32a-icosahydro-1H,12H-11,15-epimino-23,25-etheno-9,28-methano-26,30-(metheno)dipyrrolo[2,3-c:3′,4′-v][1,18]dioxa[6,9,12]triazacyclotriacontine-1-carboxylate

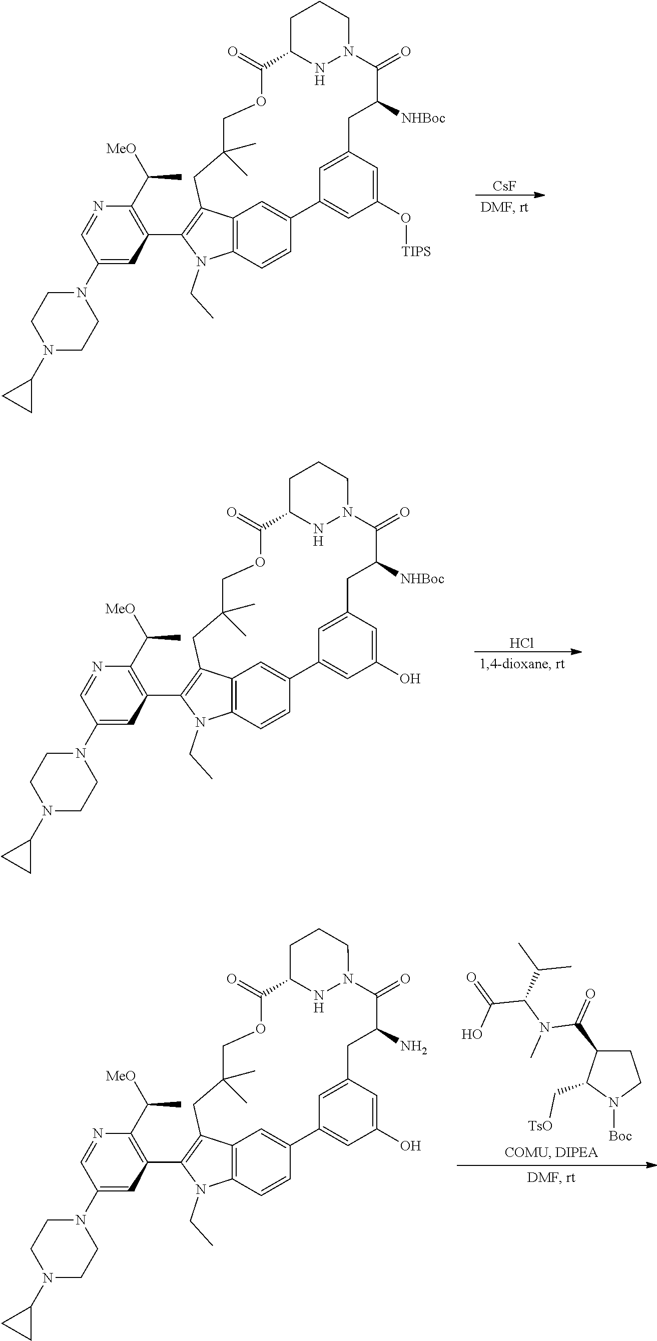

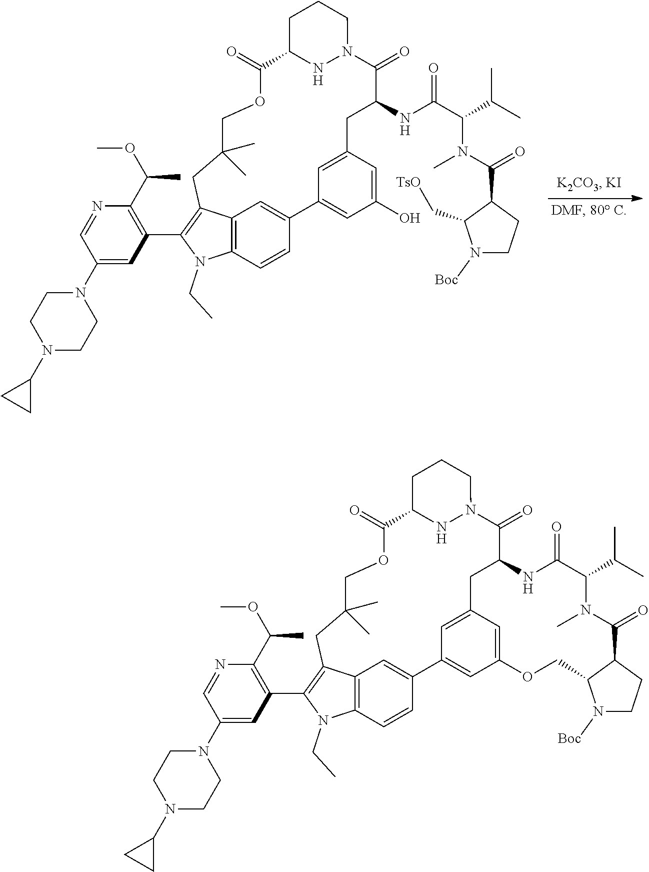

Step 1. To solution of tert-butyl ((6³S,4S)-1²-(5-(4-cyclopropylpiperazin-1-yl)-2-((S)-1-methoxyethyl)pyridin-3-yl)-1¹-ethyl-10,10-dimethyl-5,7-dioxo-2⁵-((triisopropylsilyl)oxy)-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)carbamate (10.0 g, 9.80 mmol) in DMF (100 mL) was treated with CsF (7.44 g, 49.000 mmol) for 1 h at room temperature. The reaction was quenched with H₂O at 0° C. The resulting mixture was then extracted with EtOAc (3×150 mL), washed with brine (3×150 mL), dried over anhydrous Na₂SO₄, filtered, and concentrated under reduced pressure to afford tert-butyl ((6³S,4S)-1²-(5-(4-cyclopropylpiperazin-1-yl)-2-((S)-1-methoxyethyl)pyridin-3-yl)-1¹-ethyl-2⁵-hydroxy-10,10-dimethyl-5,7-dioxo-6¹,6²,6³,6⁴,6⁵,6⁶-hexahydro-1¹H-8-oxa-1 (5,3)-indola-6(1,3)-pyridazina-2(1,3)-benzenacycloundecaphane-4-yl)carbamate (8.68 g, crude) as a yellow solid. LCMS (ESI) m/z: [M+H] calcd for C₄₉H₆₅N₇O₇: 864.5; found 854.4.

Step 2. To a stirred solution of tert-butyl ((6³S,4S)-1²-(5-(4-cyclopropylpiperazin-1-yl)-2-((S)-1-methoxyethyl)pyridin-3-yl)-1¹-ethyl-2⁵-hydroxy-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)carbamate (8.68 g, crude) in 1,4-dioxane (40 mL) was added dropwise a solution of HCl (40 mL, 4.0 M in 1,4-dioxane) at 0° C. The reaction mixture was stirred overnight at room temperature then was concentrated under reduced pressure to give (6³S,4S)-4-amino-1²-(5-(4-cyclopropylpiperazin-1-yl)-2-((S)-1-methoxyethyl)pyridin-3-yl)-1¹-ethyl-25-hydroxy-10,10-dimethyl-6¹,6²,6³,6⁴,6⁵,6⁶-hexahydro-1¹H-8-oxa-1(5,3)-indola-6(1,3)-pyridazina-2(1,3)-benzenacycloundecaphane-5,7-dione (9.7 g, crude) as a yellow solid. LCMS (ESI) m/z: [M+H] calcd for C₄₄H₅₇N₇O₅: 764.4; found 764.1.

Step 3. To a stirred solution of (6³S,4S)-4-amino-1²-(5-(4-cyclopropylpiperazin-1-yl)-2-((S)-1-methoxyethyl)pyridin-3-yl)-1¹-ethyl-25-hydroxy-10,10-dimethyl-6¹,6²,6³,6⁴,6⁵,6⁶-hexahydro-1¹H-8-oxa-1(5,3)-indola-6(1,3)-pyridazina-2(1,3)-benzenacycloundecaphane-5,7-dione (5.66 g, crude) in DMF (60 mL) were added DIPEA (38.3 g, 296 mmol), N-((2S,3S)-1-(tert-butoxycarbonyl)-2-((tosyloxy)methyl)pyrrolidine-3-carbonyl)-N-methyl-L-valine (3.80 g, 7.41 mmol) and COMU (4.76 g, 11.1 mmol) portion-wise at 0° C. The reaction mixture was stirred for 1 h at 0° C. The reaction was then quenched with H₂O at 0° C. and the resulting mixture was extracted with EtOAc (3×100 mL), washed with brine (3×300 mL), dried over anhydrous Na₂SO₄, filtered, and concentrated under reduced pressure. The residue was purified by silica gel column chromatography to give tert-butyl (2S,3S)-3-(((2S)-1-(((6³S,4S)-1²-(5-(4-cyclopropylpiperazin-1-yl)-2-((S)-1-methoxyethyl)pyridin-3-yl)-1¹-ethyl-2⁵-hydroxy-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)- benzenacycloundecaphane-4-yl)amino)-3-methyl-1-oxobutan-2-yl)(methyl)carbamoyl)-2-((tosyloxy)methyl)pyrrolidine-1-carboxylate (7.26 g, 99% yield over 3 steps) as a yellow solid. LCMS (ESI) m/z: [M+H] calcd for C₆₈H₉₁N₉O₁₂S: 1257.7; found 1257.7.

Step 4. To a stirred solution of tert-butyl (2S,3S)-3-(((2S)-1-(((6³S,4S)-1²-(5-(4-cyclopropylpiperazin-1-yl)-2-((S)-1-methoxyethyl)pyridin-3-yl)-1¹-ethyl-2⁵-hydroxy-10,10-dimethyl-5,7-dioxo-6¹,6²,6³,6⁴,6⁵,6⁶-hexahydro-1′¹H-8-oxa-1(5,3)-indola-6(1,3)-pyridazina-2(1,3)-benzenacycloundecaphane-4-yl)amino)-3-methyl-1-oxobutan-2-yl)(methyl)carbamoyl)-2-((tosyloxy)methyl)pyrrolidine-1-carboxylate (7.26 g, 5.77 mmol) in DMF (700 mL) were added K₂CO₃ (7.97 g, 57.7 mmol) and KI (0.96 g, 5.77 mmol) portion-wise at 0° C. under an atmosphere of N₂. The resulting mixture was stirred for additional 3 h at 80° C. The reaction was quenched with H₂O at 0° C. The resulting mixture was then extracted with EtOAc (3×300 mL), treated with brine (3×600 mL), dried over anhydrous Na₂SO₄, filtered, and concentrated under reduced pressure. The residue was purified by reversed-phase flash column chromatography to give tert-butyl (3aS,6S,9S,15S,32aS)-21-(5-(4-cyclopropylpiperazin-1-yl)-2-((S)-1-methoxyethyl)pyridin-3-yl)-22-ethyl-6-isopropyl-5,19,19-trimethyl-4,7,10,16-tetraoxo-2,3,3a,4,5,6,7,8,9,10,13,14,15,16,18,19,20,22,32,32a-icosahydro-1H,12H-11,15-epimino-23,25-etheno-9,28-methano-26,30-(metheno)dipyrrolo[2,3-c:3′,4′-v][1,18]dioxa[6,9,12]triazacyclotriacontine-1-carboxylate (4.95 g, 79% yield) as a yellow oil. LCMS (ESI) m/z: [M+H] calcd for C₆₁H₈₃N₉O₉: 1086.6; found 1086.3.

### Intermediate 7. Synthesis of tert-butyl ((6³S,4S)-1²-(5-(1-cyclopropylpiperidin-4-yl)-2-((S)-1-methoxyethyl)pyridin-3-yl)-10,10-dimethyl-5,7-dioxo-11-(2,2,2-trifluoroethyl)-25-((triisopropylsilyl)oxy)-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)carbamate

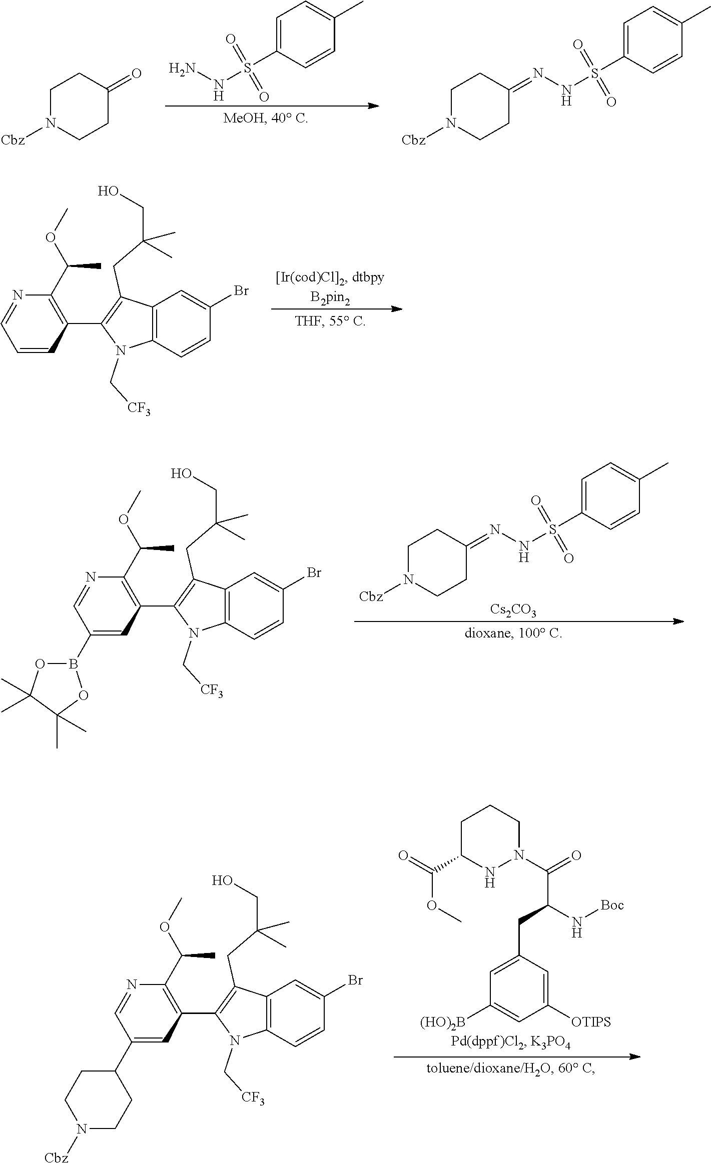

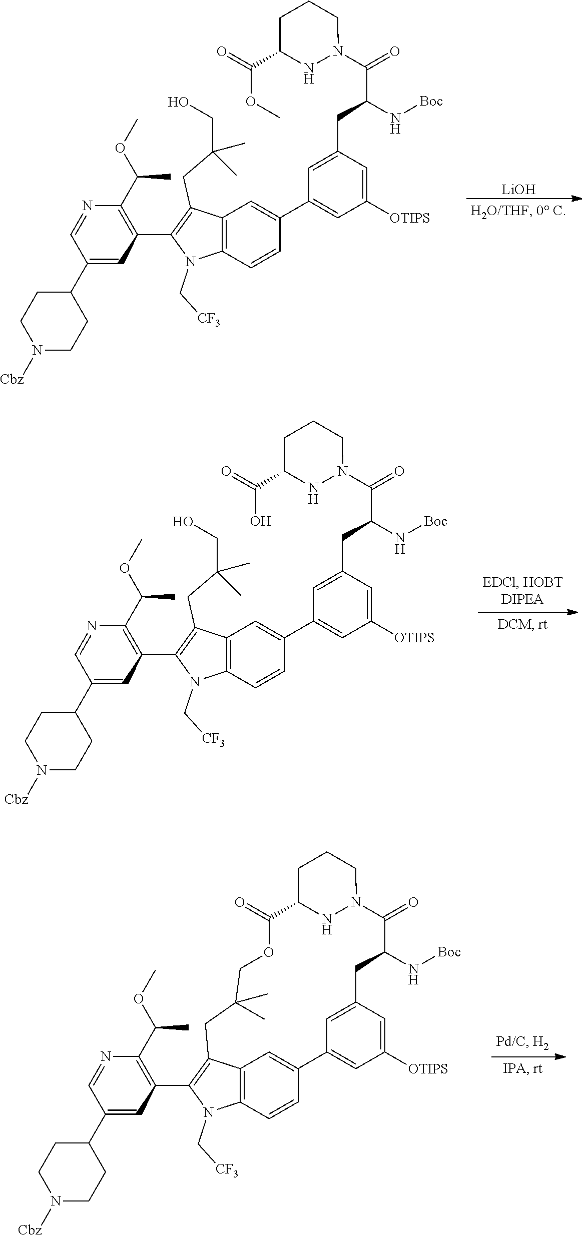

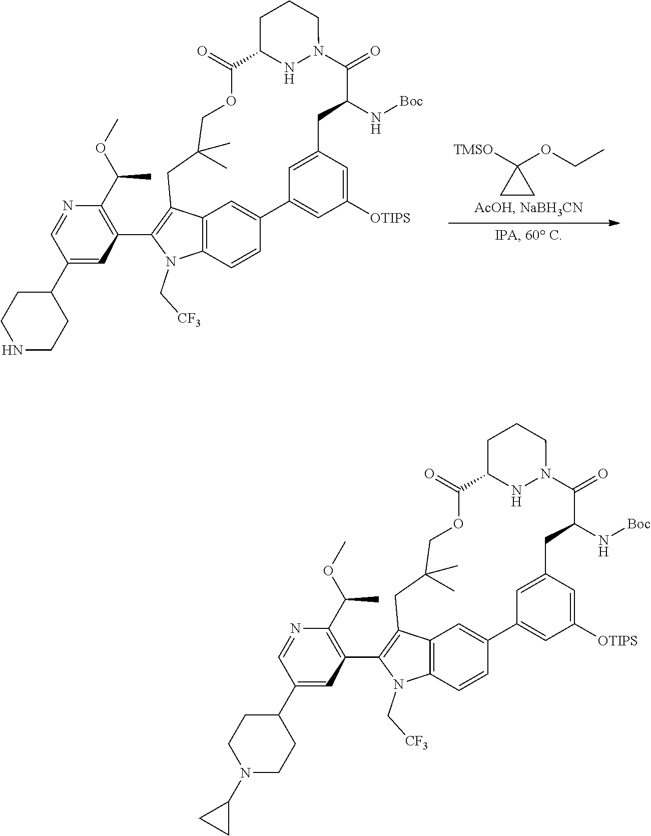

Step 1. To a solution of benzyl 4-oxopiperidine-1-carboxylate (25 g, 107 mmol) in MeOH (2.5 L) at room temperature was added 4-methylbenzenesulfonohydrazide (20 g, 107 mmol). The resulting mixture was stirred for 2 h at 40° C. and the precipitated solids were collected by filtration to afford benzyl 4-(2-tosylhydrazineylidene)piperidine-1-carboxylate (30 g, crude), which was used without further purification. LCMS (ESI) m/z: [M+H] calcd for C₂₀H₂₃N₃O₄S: 402.2; found 402.2.

Step 2. To a solution of (S)-3-(5-bromo-2-(2-(1-methoxyethyl)pyridin-3-yl)-1-(2,2,2-trifluoroethyl)-1H-indol-3-yl)-2,2-dimethylpropan-1-ol (20 g, 40.1 mmol) and 4,4,4′,4′,5,5,5′,5′-octamethyl-2,2′-bi(1,3,2-dioxaborolane) (20.3 g, 80.1 mmol) in THE (80 mL) at room temperature was added bis(15-cyclooctadiene)diiridium(I) dichloride (670 mg, 1.00 mmol) and 4,4′-di-tert-butyl-2,2′-dipyridyl (1.07 g, 4.01 mmol). The resulting mixture was stirred for 16 h at 55° C. and then concentrated under reduced pressure to afford (S)-3-(5-bromo-2-(2-(1-methoxyethyl)-5-(4,4,5,5-tetramethyl-1,3,2-dioxaborolan-2-yl)pyridin-3-yl)-1-(2,2,2-trifluoroethyl)-1H-indol-3-yl)-2,2-dimethylpropan-1-ol (20.5 g, crude), which was used without further purification. LCMS (ESI) m/z: [M-C₆H₁₀+H] calcd for C₂₉H₃₇BBrF₃N₂O₄: 543.1; found 542.9.

Step 3. To a solution of (S)-3-(5-bromo-2-(2-(1-methoxyethyl)-5-(4,4,5,5-tetramethyl-1,3,2-dioxaborolan-2-yl)pyridin-3-yl)-1-(2,2,2-trifluoroethyl)-1H-indol-3-yl)-2,2-dimethylpropan-1-ol (20.0 g, crude) and benzyl 4-(2-tosylhydrazineylidene)piperidine-1-carboxylate (12.3 g, 30.7 mmol) in dioxane (200 mL) at room temperature was added Cs₂CO₃ (19.9 g, 61.4 mmol). The resulting solution was stirred for 10 h at 100° C. The reaction was then filtered, the filter cake washed with EtOAc (3×50 mL), and the filtrate concentrated under reduced pressure. The residue was diluted with H₂O (200 mL) and the aqueous layer extracted with EtOAc (2×200 mL). The combined organic extracts were dried over Na₂SO₄, filtrated, and concentrated under reduced pressure. Purification by normal phase chromatography afforded benzyl (S)-4-(5-(5-bromo-3-(3-hydroxy-2,2-dimethylpropyl)-1-(2,2,2-trifluoroethyl)-1H-indol-2-yl)-6-(1-methoxyethyl)pyridin-3-yl)piperidine-1-carboxylate (15 g, 61% yield over 2 steps) as a yellow solid. LCMS (ESI) m/z: [M+H] calcd for C₃₆H₄₁BrF₃N₃O₃: 716.2; found 716.2.

Step 4. To a solution of benzyl (S)-4-(5-(5-bromo-3-(3-hydroxy-2,2-dimethylpropyl)-1-(2,2,2-trifluoroethyl)-1H-indol-2-yl)-6-(1-methoxyethyl)pyridin-3-yl)piperidine-1-carboxylate (15 g, 20.9 mmol) and (3-((S)-2-((tert-butoxycarbonyl)amino)-3-((S)-3-(methoxycarbonyl)tetrahydropyridazin-1(2H)-yl)-3-oxopropyl)-5-((triisopropylsilyl)oxy)phenyl)boronic acid (12.7 g, 20.9 mmol) in toluene (90 mL), dioxane (30 mL) and H₂O (30 mL) were added K₃PO₄ (8.89 g, 41.9 mmol) and Pd(dppfCl₂·DCM (1.71 g, 2.09 mmol). The resulting mixture was stirred for 12 h at 60° C. and diluted at room temperature by the addition of H₂O (200 mL). The aqueous layer was extracted with EtOAc (2×100 mL) and the combined organic extracts were dried over Na₂SO₄, filtered, and concentrated under reduced pressure to afford methyl (S)-1-((S)-3-(3-(2-(5-(1-((benzyloxy)carbonyl)piperidin-4-yl)-2-((S)-1-methoxyethyl)pyridin-3-yl)-3-(3-hydroxy-2,2-dimethylpropyl)-1-(2,2,2-trifluoroethyl)-1H-indol-5-yl)-5-((triisopropylsilyl)oxy)phenyl)-2-((tert-butoxycarbonyl)amino)propanoyl)hexahydropyridazine-3-carboxylate (27.6 g, crude), which was used without further purification. LCMS (ESI) m/z: [M+H] calcd for C₆₅H₈₉F₃N₆O₁₀Si: 1199.7; found 1199.7.

Step 5. To a solution of methyl (S)-1-((S)-3-(3-(2-(5-(1-((benzyloxy)carbonyl)piperidin-4-yl)-2-((S)-1-methoxyethyl)pyridin-3-yl)-3-(3-hydroxy-2,2-dimethylpropyl)-1-(2,2,2-trifluoroethyl)-1H-indol-5-yl)-5-((triisopropylsilyl)oxy)phenyl)-2-((tert-butoxycarbonyl)amino)propanoyl)hexahydropyridazine-3-carboxylate (27.0 g, crude) in THE (135 mL) and H₂O (135 mL) at 0° C. was added LiOH (1.08 g, 45.0 mmol). The resulting mixture was stirred for 4 h at 0° C. and then acidified to pH 6 with 1M HCl. The aqueous layer was extracted with EtOAc (3×80 mL) and the combined organic extracts were dried over Na₂SO₄ and concentrated under reduced pressure to afford (S)-1-((S)-3-(3-(2-(5-(1-((benzyloxy)carbonyl)piperidin-4-yl)-2-((S)-1-methoxyethyl)pyridin-3-yl)-3-(3-hydroxy-2,2-dimethylpropyl)-1-(2,2,2-trifluoroethyl)-1H-indol-5-yl)-5-((triisopropylsilyl)oxy)phenyl)-2-((tert-butoxycarbonyl)amino)propanoyl)hexahydropyridazine-3-carboxylic acid (25.0 g, crude), which was used without further purification. LCMS (ESI) m/z: [M+H] calcd for C₆₄H₈₇F₃N₆O₁₀Si: 1185.6; found 1185.7.

Step 6. To a solution of (S)-1-((S)-3-(3-(2-(5-(1-((benzyloxy)carbonyl)piperidin-4-yl)-2-((S)-1-methoxyethyl)pyridin-3-yl)-3-(3-hydroxy-2,2-dimethylpropyl)-1-(2,2,2-trifluoroethyl)-1H-indol-5-yl)-5-((triisopropylsilyl)oxy)phenyl)-2-((tert-butoxycarbonyl)amino)propanoyl)hexahydropyridazine-3-carboxylic acid (25.0 g, crude) and DIPEA (81.8 g, 632 mmol) in DCM (2.50 L) at 0° C. were added EDCI (115 g, 601 mmol) and HOBT (14.3 g, 105 mmol). The resulting solution was stirred overnight at room temperature, concentrated under reduced pressure, and the residue diluted with sat. aq. NH₄Cl (300 mL). The aqueous mixture was extracted with EtOAc (2×300 mL), and the combined organic extracts were dried over Na₂SO₄ and concentrated under reduced pressure. Purification by normal phase chromatography afforded benzyl 4-(5-((6³S,4S)-4-((tert-butoxycarbonyl)amino)-10,10-dimethyl-5,7-dioxo-11-(2,2,2-trifluoroethyl)-2⁵-((triisopropylsilyl)oxy)-6¹,6²,6³,6⁴,6⁵,6⁶-hexahydro-1¹H-8-oxa-1(5,3)-indola-6(1,3)-pyridazina-2(1,3)-benzenacycloundecaphane-1²-yl)-6-((S)-1-methoxyethyl)pyridin-3-yl)piperidine-1-carboxylate (10.0 g, 42% yield over 3 steps) as a yellow oil. LCMS (ESI) m/z: [M+H] calcd for C₆₄H₈₅F₃N₆O₉Si: 1167.6; found 1167.7.

Step 7. To a solution of benzyl 4-(5-((6³S,4S)-4-((tert-butoxycarbonyl)amino)-10,10-dimethyl-5,7-dioxo-1¹-(2,2,2-trifluoroethyl)-2⁵-((triisopropylsilyl)oxy)-6¹,6²,6³,6⁴,6⁵,6⁶-hexahydro-1¹H-8-oxa-1(5,3)-indola-6(1,3)-pyridazina-2(1,3)-benzenacycloundecaphane-1²-yl)-6-((S)-1-methoxyethyl)pyridin-3-yl)piperidine-1-carboxylate (10 g, 8.57 mmol) in IPA (100 mL) was added Pd/C (5.0 g, 10 wt % Pd). The resulting mixture was stirred overnight at room temperature under an atmosphere of H₂, filtered, and the filter cake washed with EtOAc (3×50 mL). The filtrate was concentrated under reduced pressure to afford tert-butyl ((6³S,4S)-1²-(2-((S)-1-methoxyethyl)-5-(piperidin-4-yl)pyridin-3-yl)-10,10-dimethyl-5,7-dioxo-1¹-(2,2,2-trifluoroethyl)-2⁵-((triisopropylsilyl)oxy)-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)carbamate (9.70 g, crude), which was used without further purification. LCMS (ESI) m/z: [M+H] calcd for C₅₆H₇₉F₃N₆O₇Si: 1033.6; found 1034.7.

Step 8. To a solution of tert-butyl ((6³S,4S)-1²-(2-((S)-1-methoxyethyl)-5-(piperidin-4-yl)pyridin-3-yl)-10,10-dimethyl-5,7-dioxo-1¹-(2,2,2-trifluoroethyl)-2⁵-((triisopropylsilyl)oxy)-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)carbamate (9.30 g, crude) in IPA (93 mL) at 0° C. were added (1-ethoxycyclopropoxy)trimethylsilane (4.71 g, 27.0 mmol) and AcOH (1.62 g, 27.0 mmol) followed by NaBH₃CN (1.70 g, 27.0 mmol). The resulting mixture was stirred overnight at 60° C. and the reaction was then quenched by the addition sat. aq. NaHCO₃ (200 mL). The aqueous mixture was extracted with EtOAc (2×100 mL), and the combined organic extracts were dried over Na₂SO₄ and concentrated under reduced pressure. Purification by normal phase chromatography afforded tert-butyl ((6³S,4S)-1²-(5-(1-cyclopropylpiperidin-4-yl)-2-((S)-1-methoxyethyl)pyridin-3-yl)-10,10-dimethyl-5,7-dioxo-11-(2,2,2-trifluoroethyl)-25-((triisopropylsilyl)oxy)-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)carbamate (5.10 g, 58% yield over 2 steps) as a yellow oil. LCMS (ESI) m/z: [M+H] calcd for C₅₆H₇₉F₃N₆O₇Si: 1073.6; found 1074.7.

### Intermediate 8. Synthesis of tert-butyl (3aS,6S,9S,15S,32aS)-21-(5-(1-cyclopropylpiperidin-4-yl)-2-((S)-1-methoxyethyl)pyridin-3-yl)-6-isopropyl-5,19,19-trimethyl-4,7,10,16-tetraoxo-22-(2,2,2-trifluoroethyl)-2,3,3a,4,5,6,7,8,9,10,13,14,15,16,18,19,20,22,32,32a-icosahydro-1H,12H-11,15-epimino-23,25-etheno-9,28-methano-26,30-(metheno)dipyrrolo[2,3-c:3′,4′-v][1,18]dioxa[6,9,12]triazacyclotriacontine-1-carboxylate

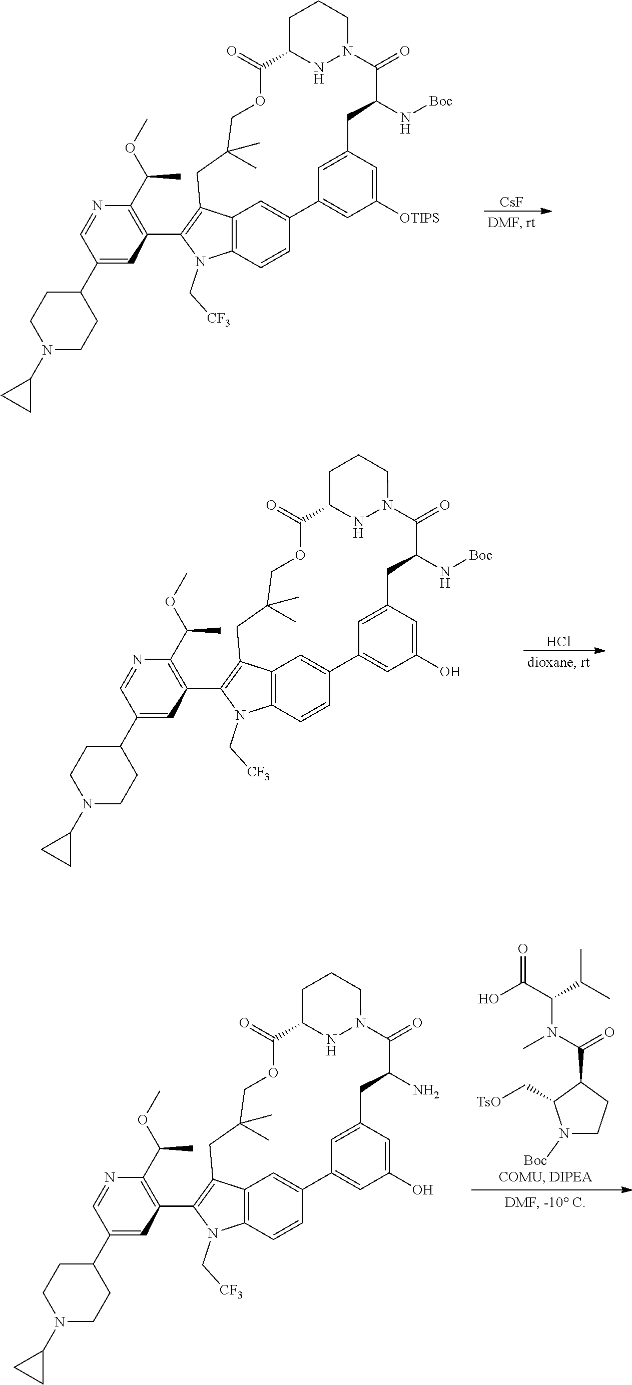

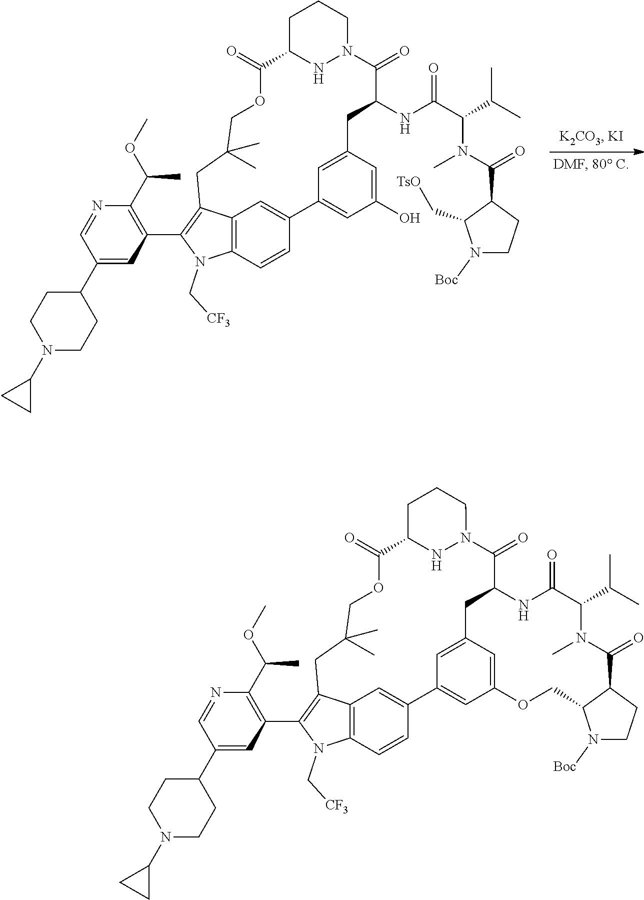

Step 1. To a solution of tert-butyl ((6³S,4S)-1²-(5-(1-cyclopropylpiperidin-4-yl)-2-((S)-1-methoxyethyl)pyridin-3-yl)-10,10-dimethyl-5,7-dioxo-11-(2,2,2-trifluoroethyl)-25-((triisopropylsilyl)oxy)-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)carbamate (5.1 g, 4.75 mmol) in DMF (50 mL) was added CsF (3.61 g, 23.8 mmol). The resulting mixture was stirred for 1 h at room temperature and the reaction was then quenched at 0° C. by the addition of H₂O. The aqueous mixture was extracted with EtOAc (2×50 mL), and the combined organic extracts were washed with brine, dried over Na₂SO₄, filtered, and concentrated under reduced pressure. Purification by normal phase chromatography afforded tert-butyl ((6³S,4S)-1²-(5-(1-cyclopropylpiperidin-4-yl)-2-((S)-1-methoxyethyl)pyridin-3-yl)-2⁵-hydroxy-10,10-dimethyl-5,7-dioxo-1¹-(2,2,2-trifluoroethyl)-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)carbamate (3.20 g, 73% yield) as a light yellow solid. LCMS (ESI) m/z: [M+H] calcd for C₅₀H₆₃F₃N₆O₇: 917.5; found 917.5.

Step 2. To a solution of tert-butyl ((6³S,4S)-1²-(5-(1-cyclopropylpiperidin-4-yl)-2-((S)-1-methoxyethyl)pyridin-3-yl)-2⁵-hydroxy-10,10-dimethyl-5,7-dioxo-11-(2,2,2-trifluoroethyl)-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)carbamate (3.2 g, 3.50 mmol) in dioxane (15 mL) at 0° C. was added a 4 M solution of HCl in dioxane (15 mL). The resulting mixture was stirred for 1 h at room temperature and then concentrated under reduced pressure. The residue was diluted with H₂O (10 mL) and the resulting solution basified to pH 8 with sat. aq. NaHCO₃. The aqueous mixture was extracted with DCM (3×30 mL) and the combined organic extracts were dried over Na₂SO₄, filtered, and concentrated under reduced pressure to afford (6³S,4S)-4-amino-1²-(5-(1-cyclopropylpiperidin-4-yl)-2-((S)-1-methoxyethyl)pyridin-3-yl)-2⁵-hydroxy-10,10-dimethyl-11-(2,2,2-trifluoroethyl)-6¹,6²,6³,6⁴,6⁵,6⁶-hexahydro-1¹H-8-oxa-1(5,3)-indola-6(1,3)-pyridazina-2(1,3)-benzenacycloundecaphane-5,7-dione (2.54 g, crude) which was used without further purification. LCMS (ESI) m/z: [M+H] calcd for C₄₅H₅₅F₃N₆O₅: 817.4; found 817.3.

Step 3. To a solution of (6³S,4S)-4-amino-1²-(5-(1-cyclopropylpiperidin-4-yl)-2-((S)-1-methoxyethyl)pyridin-3-yl)-2⁵-hydroxy-10,10-dimethyl-1¹-(2,2,2-trifluoroethyl)-6¹,6²,6³,6⁴,6⁵,6⁶-hexahydro-1¹H-8-oxa-1(5,3)-indola-6(1,3)-pyridazina-2(1,3)-benzenacycloundecaphane-5,7-dione (2.52 g, crude) and N-((2S,3S)-1-(tert-butoxycarbonyl)-2-((tosyloxy)methyl)pyrrolidine-3-carbonyl)-N-methyl-L-valine (2.37 g, 4.62 mmol) in DMF (20 mL) at −10° C. were added DIPEA (21.5 mL, 123 mmol) and COMU (1.85 g, 4.32 mmol). The resulting mixture was stirred for 1 h at −10° C. and the reaction was then quenched at 0° C. by the addition of cold H₂O. The aqueous mixture was extracted with EtOAc (2×10 mL), and the combined organic extracts were washed with brine, dried over Na₂SO₄, filtrated, and concentrated under reduced pressure. Purification by normal phase chromatography afforded tert-butyl (2S,3S)-3-(((2S)-1-(((6³S,4S)-12-(5-(1-cyclopropylpiperidin-4-yl)-2-((S)-1-methoxyethyl)pyridin-3-yl)-2⁵-hydroxy-10,10-dimethyl-5,7-dioxo-1¹-(2,2,2-trifluoroethyl)-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)amino)-3-methyl-1-oxobutan-2-yl)(methyl)carbamoyl)-2-((tosyloxy)methyl)pyrrolidine-1-carboxylate (1.90 g, 41% yield over 2 steps) as a yellow solid. LCMS (ESI) m/z: [M+H] calcd for C₆₉H₈₉F₃N₈O₁₂S: 1311.6; found 1311.5.

Step 4. To a solution of tert-butyl (2S,3S)-3-(((2S)-1-(((6³S,4S)-12-(5-(1-cyclopropylpiperidin-4-yl)-2-((S)-1-methoxyethyl)pyridin-3-yl)-2⁵-hydroxy-10,10-dimethyl-5,7-dioxo-1¹-(2,2,2-trifluoroethyl)-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)amino)-3-methyl-1-oxobutan-2-yl)(methyl)carbamoyl)-2-((tosyloxy)methyl)pyrrolidine-1-carboxylate (1.33 g, 1.01 mmol) in DMF (130 mL) were added K₂CO₃ (1.40 g, 10.1 mmol) and KI (168 mg, 1.01 mmol). The resulting mixture was stirred for 4 h at 80° C. and the reaction was then quenched at 0° C. by the addition of cold H₂O. The aqueous mixture was extracted with EtOAc (2×100 mL), and the combined organic extracts were washed with brine, dried over Na₂SO₄, filtered, and concentrated under reduced pressure. Purification by reversed phase chromatography afforded tert-butyl (3aS,6S,9S,15S,32aS)-21-(5-(1-cyclopropylpiperidin-4-yl)-2-((S)-1-methoxyethyl)pyridin-3-yl)-6-isopropyl-5,19,19-trimethyl-4,7,10,16-tetraoxo-22-(2,2,2-trifluoroethyl)-2,3,3a,4,5,6,7,8,9,10,13,14,15,16,18,19,20,22,32,32a-icosahydro-1H,12H-11,15-epimino-23,25-etheno-9,28-methano-26,30-(metheno)dipyrrolo[2,3-c:3′,4′-v][1,18]dioxa[6,9,12]triazacyclotriacontine-1-carboxylate (600 mg, 42% yield) as a yellow solid. LCMS (ESI) m/z: [M+H] calcd for C₆₂H₈₁F₃N₈O₉: 1139.6; found 1139.9.

### Synthesis of Exemplary Compounds

### Examples A25 and A26. Synthesis of (9S,15S,18S,22S)-2-ethyl-18-isopropyl-3-(2-((S)-1-methoxyethyl)-5-(4-methylpiperazin-1-yl)pyridin-3-yl)-5,5,19-trimethyl-8,14,17,20-tetraoxo-2,4,5,6,9,10,11,12,14,15,16,17,18,19,20,21,22,23-octadecahydro-8H-9,13-epimino-1,30-etheno-15,27-methano-25,29-(metheno)pyrrolo[3,4-v][1,18]dioxa[6,9,12]triazacyclotriacontine-22-carbonitrile and (9S,15S,18S,22R)-2-ethyl-18-isopropyl-3-(2-((S)-1-methoxyethyl)-5-(4-methylpiperazin-1-yl)pyridin-3-yl)-5,5,19-trimethyl-8,14,17,20-tetraoxo-2,4,5,6,9,10,11,12,14,15,16,17,18,19,20,21,22,23-octadecahydro-8H-9,13-epimino-1,30-etheno-15,27-methano-25,29-(metheno)pyrrolo[3,4-v][1,18]dioxa[6,9,12]triazacyclotriacontine-22-carbonitrile

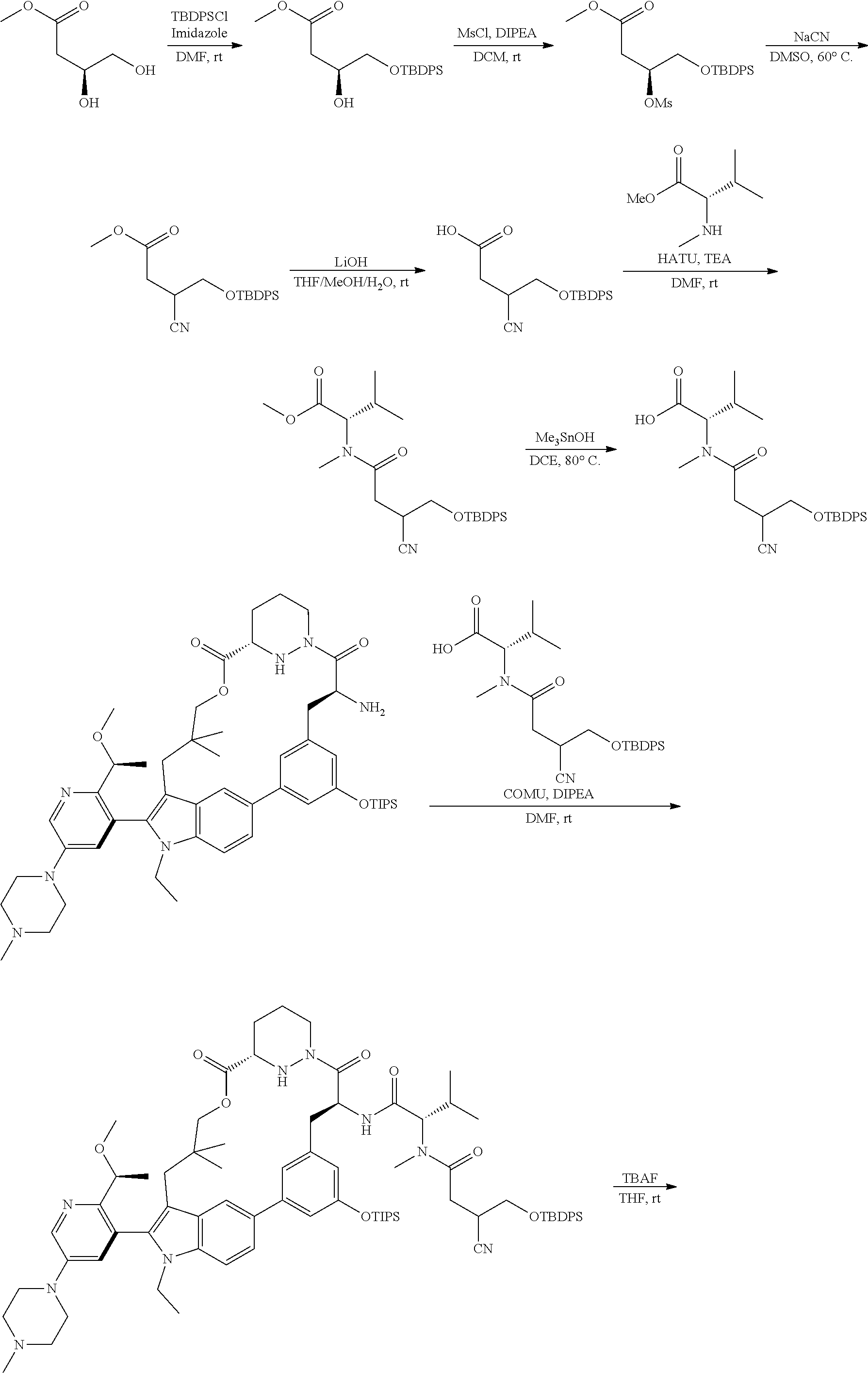

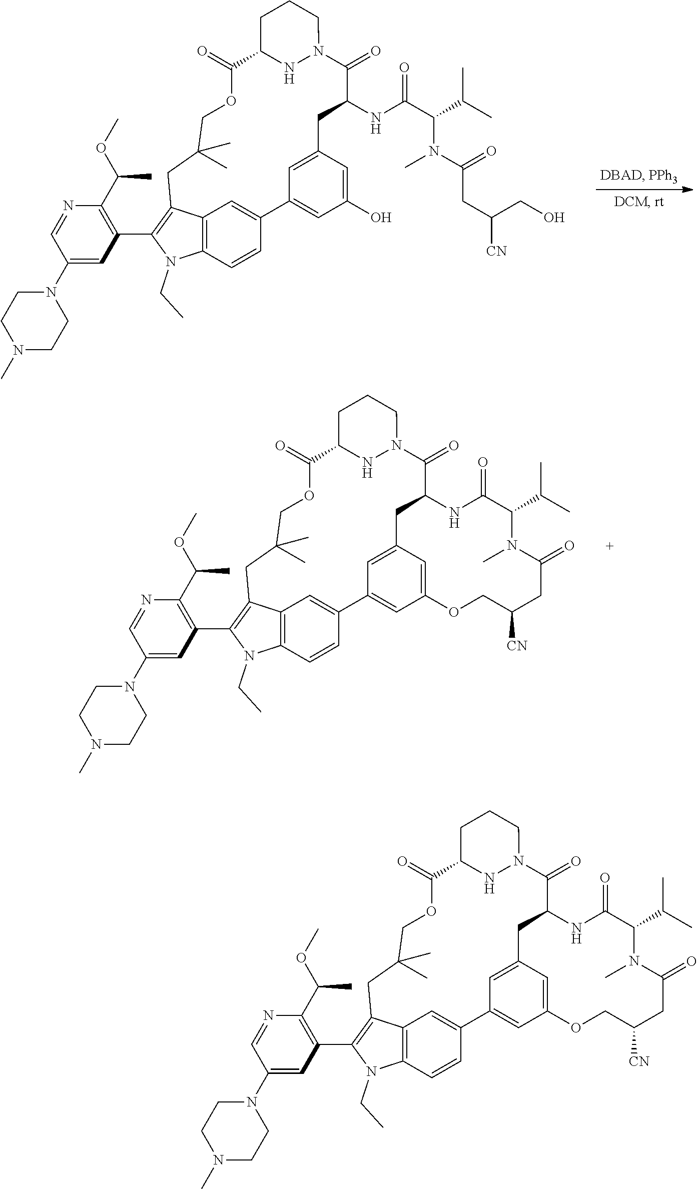

Step 1. To a solution of methyl (S)-3,4-dihydroxybutanoate (1.0 g, 7.5 mmol) and imidazole (1.02 g, 14.9 mmol) in DMF (10 mL) stirred at 0° C. was added TBDPSCI (2.05 g, 7.46 mmol). The resulting mixture was stirred for 3 h at room temperature and the reaction was quenched at 0° C. by the addition of cold H₂O. The aqueous mixture was extracted with EtOAc (3×50 mL), and the combined organic extracts were washed with water, dried over Na₂SO₄, filtered, concentrated under reduced pressure. Purification by normal phase chromatography afforded methyl (S)-4-((tert-butyldiphenylsilyl)oxy)-3-hydroxybutanoate (2.4 g, 86% yield) as a clear oil. LCMS (ESI) m/z: [M+Na] calcd for C₂₁H₂₈O₄Si: 395.2; found 395.2.

Step 2. To a solution of methyl (S)-4-((tert-butyldiphenylsilyl)oxy)-3-hydroxybutanoate (1.0 g, 2.7 mmol) and DIPEA (1.04 g, 8.05 mmol) in DCM (20 mL) at 0° C. was added MsCl (461 mg, 4.03 mmol). The resulting mixture was stirred for 1 h at room temperature and the reaction quenched at 0° C. by the addition of cold H₂O. The aqueous layer was extracted with DCM (2×50 mL) and the combined organic extracts were washed with H₂O, dried over Na₂SO₄, filtered, and concentrated under reduced pressure to afford methyl (S)-4-((tert-butyldiphenylsilyl)oxy)-3-((methylsulfonyl)oxy)butanoate (1.38 g, crude) which was taken to the next reaction without further purification. LCMS (ESI) m/z: [M+H] calcd for C₂₂H₃₀O₆SSi: 451.2; found 451.2.

Step 3. To a solution of methyl (S)-4-((tert-butyldiphenylsilyl)oxy)-3-((methylsulfonyl)oxy)butanoate (1.38 g, crude) in DMSO (15 mL) stirred at 55° C. was added NaCN (750 mg, 15.3 mmol). The resulting mixture was stirred for 2 h at 60° C. and the reaction quenched at 0° C. by the addition of cold H₂O. The aqueous mixture was extracted with EtOAc (2×50 mL), dried over Na₂SO₄, filtered, and concentrated under reduced pressure. Purification by normal phase chromatography afforded methyl 4-((tert-butyldiphenylsilyl)oxy)-3-cyanobutanoate (600 mg, 20% yield over 2 steps) as a brown solid. LCMS (ESI) m/z: [M+H] calcd for C₂₂H₂₇O₃Si: 382.2; found 382.2.

Step 4. To a solution of methyl 4-((tert-butyldiphenylsilyl)oxy)-3-cyanobutanoate (600 mg, 1.57 mmol) in THE (6.0 mL) and MeOH (6.0 mL) at 0° C. was added a solution of LiOH·H₂O (99.0 mg, 2.36 mmol) into H₂O (6.0 mL). The resulting mixture was stirred for 2 h at room temperature and then concentrated under reduced pressure to afford 4-((tert-butyldiphenylsilyl)oxy)-3-cyanobutanoic acid (620 mg, crude) which was used without further purification. LCMS (ESI) m/z: [M+H] calcd for C₂₁H₂₅NO₃Si: 368.2; found 368.2.

Step 5. To a solution methyl N-methyl-L-valinate (558 mg, 3.84 mmol) and Et₃N (971 mg, 9.60 mmol) in DMF (10 mL) at 0° C. was added 4-((tert-butyldiphenylsilyl)oxy)-3-cyanobutanoic acid (1.18 g, crude) and HATU (1.83 g, 4.80 mmol). The resulting mixture was stirred for 1.5 h at room temperature and the reaction was then quenched by the addition of water. The aqueous mixture was extracted with EtOAc (3×20 mL), and the combined organic extracts were washed with brine, dried over Na₂SO₄, filtered, and concentrated under reduced pressure. Purification by normal phase chromatography afforded methyl N-(4-((tert-butyldiphenylsilyl)oxy)-3-cyanobutanoyl)-N-methyl-L-valinate (770 mg, 99% yield over 2 steps) as a white solid. LCMS (ESI) m/z: [M+H] calcd for C₂₈H₃₈N₂O₄Si: 495.3; found 495.4.

Step 6. To a solution methyl N-(4-((tert-butyldiphenylsilyl)oxy)-3-cyanobutanoyl)-N-methyl-L-valinate (100 mg, 0.202 mmol) in DCE (2.0 mL) at 80° C. was added trimethylstannanol (292 mg, 1.62 mmol). The resulting mixture was stirred at 80° C. for 15 h and concentrated under reduced pressure. Purification by normal phase prep-TLC afforded N-(4-((tert-butyldiphenylsilyl)oxy)-3-cyanobutanoyl)-N-methyl-L-valine (86 mg, 89% yield) as a white solid. LCMS (ESI) m/z: [M+H] calcd for C₂₇H₃₆N₂O₄Si: 481.3; found 481.2.

Step 7. To a solution of (6³S,4S)-4-amino-1′-ethyl-1²-(2-((S)-1-methoxyethyl)-5-(4-methylpiperazin-1-yl)pyridin-3-yl)-10,10-dimethyl-2⁵-((triisopropylsilyl)oxy)-6¹,6²,6³,6⁴,6⁵,6⁶-hexahydro-1¹H-8-oxa-1(5,3)-indola-6(1,3)-pyridazina-2(1,3)-benzenacycloundecaphane-5,7-dione (210 mg, 0.235 mmol) and DIPEA (607 mg, 4.70 mmol) in DCM (2.0 mL) at 0° C. was added N-(4-((tert-butyldiphenylsilyl)oxy)-3-cyanobutanoyl)-N-methyl-L-valine (169 mg, 0.352 mmol) and COMU (151 mg, 0.352 mmol). The resulting mixture was stirred for 1 h at room temperature and the reaction quenched at 0° C. by the addition of cold H₂O. The aqueous mixture was extracted with DCM (3×5 mL) and the combined organic extracts were dried over Na₂SO₄, filtered, and concentrated under reduced pressure. Purification by normal phase prep-TLC afforded 4-((tert-butyldiphenylsilyl)oxy)-3-cyano-N-((2S)-1-(((6³S,4S)-1¹-ethyl-1²-(2-((S)-1-methoxyethyl)-5-(4-methylpiperazin-1-yl)pyridin-3-yl)-10,10-dimethyl-5,7-dioxo-25-((triisopropylsilyl)oxy)-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)amino)-3-methyl-1-oxobutan-2-yl)-N-methylbutanamide (180 mg, 57% yield) as a white solid. LCMS (ESI) m/z: [M+H] calcd for C₇₈H₁₀₉N₉O₈Si₂: 1356.8; found 1357.2.

Step 8. To a solution of 4-((tert-butyldiphenylsilyl)oxy)-3-cyano-N-((2S)-1-(((6³S,4S)-1¹-ethyl-1²-(2-((S)-1-methoxyethyl)-5-(4-methylpiperazin-1-yl)pyridin-3-yl)-10,10-dimethyl-5,7-dioxo-25-((triisopropylsilyl)oxy)-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)amino)-3-methyl-1-oxobutan-2-yl)-N-methylbutanamide (180 mg, 0.133 mmol) in THE (2.0 mL) at 0° C. was added TBAF (69.4 mg, 0.266 mmol). The resulting mixture was stirred for 2 h at room temperature and concentrated under reduced pressure. Purification by normal phase preparative TLC (20% MeOH/DCM) afforded 3-cyano-N-((2S)-1-(((6³S,4S)-1¹-ethyl-25-hydroxy-1²-(2-((S)-1-methoxyethyl)-5-(4-methylpiperazin-1-yl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)amino)-3-methyl-1-oxobutan-2-yl)-4-hydroxy-N-methylbutanamide (117 mg, 92% yield) as a white solid. LCMS (ESI) m/z: [M+H] calcd for C₅₃H₇₁N₉O₈: 962.6; found 962.5.

Step 9. To a solution 3-cyano-N-((2S)-1-(((6³S,4S)-1¹-ethyl-25-hydroxy-1²-(2-((S)-1-methoxyethyl)-5-(4-methylpiperazin-1-yl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)amino)-3-methyl-1-oxobutan-2-yl)-4-hydroxy-N-methylbutanamide (260 mg, 0.270 mmol) and PPh₃ (354 mg, 1.35 mmol) in DCM (3.0 mL) at 0° C. was added DBAD (311 mg, 1.35 mmol). The resulting mixture was stirred for 6 h at room temperature and then concentrated under reduced pressure. Purification normal phase prep-TLC afforded a mixture of the desired products. The diastereomers were then separated by reversed phase chromatography to afford (9S,15S,18S,22S)-2-ethyl-18-isopropyl-3-(2-((S)-1-methoxyethyl)-5-(4-methylpiperazin-1-yl)pyridin-3-yl)-5,5,19-tri methyl-8,14,17,20-tetraoxo-2,4,5,6,9,10,11,12,14,15,16,17,18,19,20,21,22,23-octadecahydro-8H-9,13-epimino-1,30-etheno-15,27-methano-25,29-(metheno)pyrrolo[3,4-v][1,18]dioxa[6,9,12]triazacyclotriacontine-22-carbonitrile (11.8 mg, 4.6% yield, assumed configuration) and (9S,15S,18S,22R)-2-ethyl-18-isopropyl-3-(2-((S)-1-methoxyethyl)-5-(4-methylpiperazin-1-yl)pyridin-3-yl)-5,5,19-tri methyl-8,14,17,20-tetraoxo-2,4,5,6,9,10,11,12,14,15,16,17,18,19,20,21,22,23-octadecahydro-8H-9,13-epimino-1,30-etheno-15,27-methano-25,29-(metheno)pyrrolo[3,4-v][1,18]dioxa[6,9,12]triazacyclotriacontine-22-carbonitrile (13.0 mg, 5.1% yield, assumed configuration), each as a white solid.

Data for (9S,15S,18S,22S)-2-ethyl-18-isopropyl-3-(2-((S)-1-methoxyethyl)-5-(4-methylpiperazin-1-yl)pyridin-3-yl)-5,5,19-trimethyl-8,14,17,20-tetraoxo-2,4,5,6,9,10,11,12,14,15,16,17,18,19,20,21,22,23-octadecahydro-8H-9,13-epimino-1,30-etheno-15,27-methano-25,29-(metheno)pyrrolo[3,4-v][1,18]dioxa[6,9,12]triazacyclotriacontine-22-carbonitrile: LCMS (ESI) m/z: [M+H] calcd for C₅₃H₆₉N₉O₇: 944.5; found 945.2; ¹H NMR (400 MHz, DMSO-d₆) δ 9.33 (s, 1H), 9.28 (s, 1H), 8.72 (d, J=8.2 Hz, 1H), 8.46 (d, J=2.9 Hz, 2H), 8.27 (d, J=7.8 Hz, 1H), 7.91 (d, J=6.8 Hz, 2H), 7.58 (d, J=8.6 Hz, 2H), 7.51 (d, J=8.9 Hz, 2H), 7.33-7.21 (m, 6H), 7.18 (d, J=1.6 Hz, 1H), 7.01 (d, J=10.6 Hz, 2H), 6.50 (s, 2H), 5.37 (d, J=11.0 Hz, 4H), 4.67 (d, J=10.7 Hz, 1H), 4.30 (d, J=12.0 Hz, 2H), 4.14-4.04 (m, 1H), 3.83 (d, J=10.4 Hz, 2H), 3.65 (d, J=10.7 Hz, 2H), 3.61-3.52 (m, 2H), 3.07 (d, J=7.7 Hz, 6H), 2.87 (d, J=14.2 Hz, 1H), 2.77 (d, J=11.5 Hz, 6H), 2.70 (s, 1H), 2.46 (s, 4H), 2.22 (s, 6H), 2.01 (s, 1H), 1.95 (dd, J=10.8, 1.5 Hz, 5H), 1.80 (s, 2H), 1.64 (s, 3H), 1.54 (d, J=12.4 Hz, 2H), 1.35 (d, J=6.1 Hz, 6H), 1.24 (s, 1H), 0.99 (t, J=6.8 Hz, 6H), 0.90 (dd, J=11.6, 6.5 Hz, 5H), 0.80-0.70 (m, 10H), 0.56 (d, J=11.3 Hz, 6H).

Data for (9S,15S,18S,22R)-2-ethyl-18-isopropyl-3-(2-((S)-1-methoxyethyl)-5-(4-methylpiperazin-1-yl)pyridin-3-yl)-5,5,19-trimethyl-8,14,17,20-tetraoxo-2,4,5,6,9,10,11,12,14,15,16,17,18,19,20,21,22,23-octadecahydro-8H-9,13-epimino-1,30-etheno-15,27-methano-25,29-(metheno)pyrrolo[3,4-v][1,18]dioxa[6,9,12]triazacyclotriacontine-22-carbonitrile: LCMS (ESI) m/z: [M+H] calcd for C₅₃H₆₉N₉O₇: 944.5; found 944.8. ¹H NMR (400 MHz, DMSO-d₆) δ 9.33 (s, 1H), 9.28 (s, 1H), 8.61 (d, J=8.4 Hz, 1H), 8.45 (d, J=2.9 Hz, 2H), 8.12 (d, J=7.9 Hz, 1H), 7.91 (s, 2H), 7.58 (d, J=8.6 Hz, 2H), 7.51 (d, J=8.6 Hz, 2H), 7.33 (s, 1H), 7.24 (s, 3H), 7.17 (s, 1H), 7.01 (s, 2H), 6.50 (s, 2H), 5.35 (s, 5H), 4.69 (d, J=10.8 Hz, 1H), 4.29 (d, J=12.9 Hz, 2H), 4.10 (dd, J=12.4, 6.0 Hz, 2H), 3.90 (d, J=10.0 Hz, 1H), 3.64 (s, 1H), 3.07 (d, J=13.4 Hz, 6H), 2.88 (d, J=13.7 Hz, 1H), 2.81 (s, 6H), 2.46 (s, 5H), 2.21 (d, J=4.3 Hz, 9H), 2.11 (d, J=1.7 Hz, 2H), 1.99 (s, 3H), 1.80 (s, 1H), 1.35 (d, J=6.4 Hz, 5H), 1.24 (s, 3H), 0.98 (s, 1H), 0.99-0.88 (m, 5H), 0.81-0.74 (m, 10H), 0.58 (s, 2H), 0.53 (s, 3H).

### Example A43. Synthesis of (9S,15S,18S)-23-acetyl-2-ethyl-18-isopropyl-3-(2-((S)-1-methoxyethyl)pyridin-3-yl)-5,5,19-trimethyl-2,4,5,6,9,10,11,12,15,16,18,19,22,23,24,25-hexadecahydro-8H-9,13-epimino-1,32-etheno-15,29-methano-27,31-(metheno)pyrrolo[3,4-x][1,20]dioxa[4,8,11,14]tetraazacyclodotriacontine-8,14,17,20(21H)-tetraone

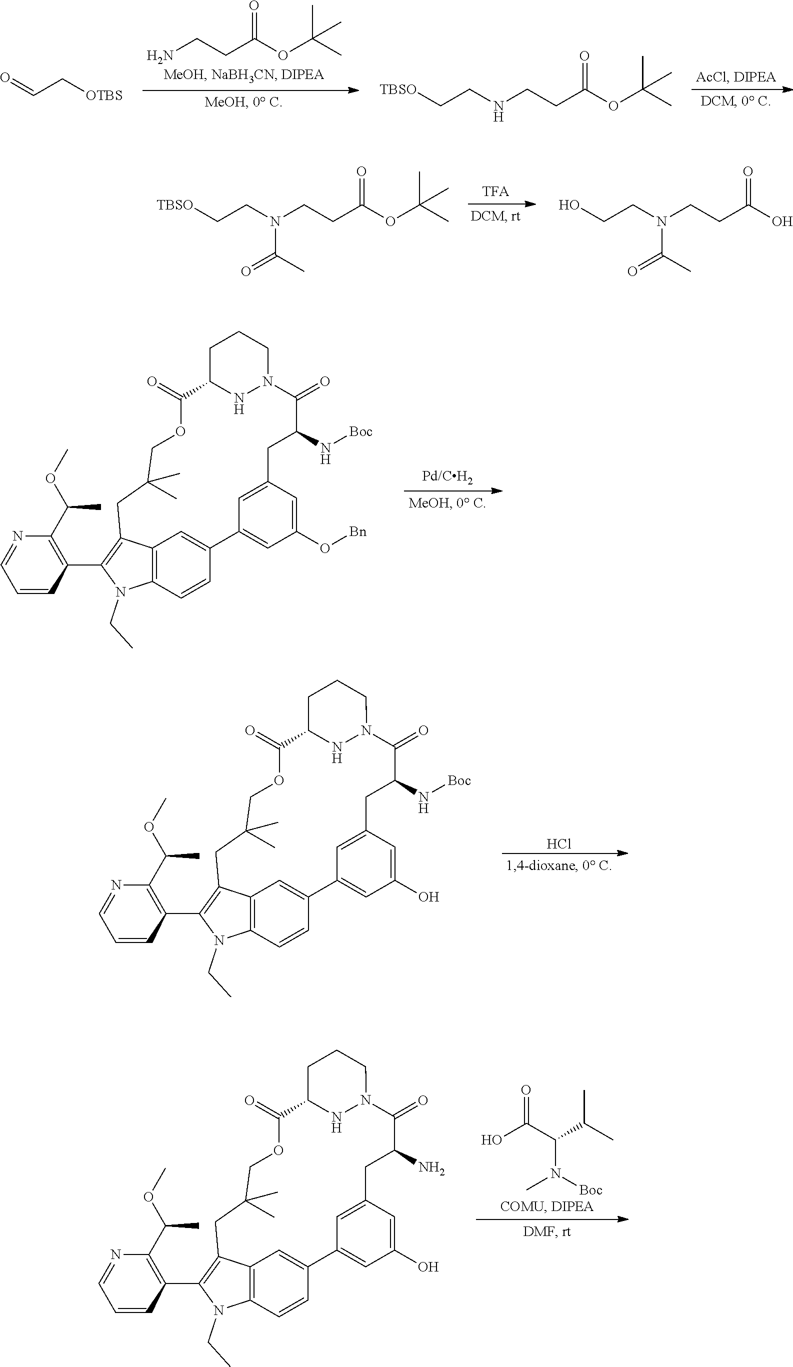

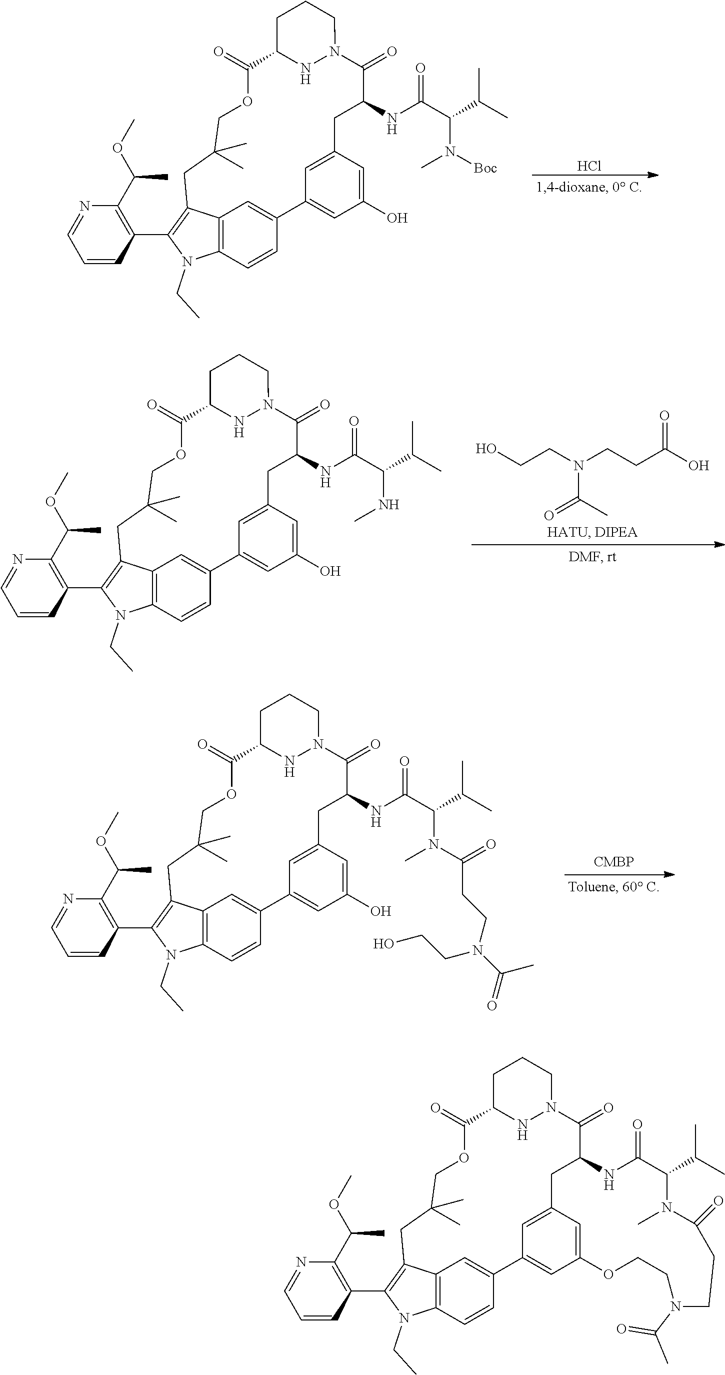

Step 1. To a solution of 2-[(tert-butyldimethylsilyl)oxy]acetaldehyde (2.00 g, 11.5 mmol), tert-butyl 3-aminopropanoate (2.50 g, 17.2 mmol), and DIPEA (2.97 g, 22.9 mmol) in MeOH (20 mL) was added NaBH₃CN (2.16 g, 34.4 mmol) at 0° C. The resulting mixture was stirred for 5 h at room temperature and was then extracted with DCM, washed with brine (3×50 mL), dried over Na₂SO₄, filtered, and concentrated under reduced pressure to give tert-butyl 3-({2-[(tert-butyldimethylsilyl)oxy]ethyl}amino)propanoate (2.00 g, crude), which was taken to the next reaction without further purification. LCMS (ESI) m/z: [M+H] calcd for C₁₅H₃₃NO₃Si: 304.2; found 304.2.

Step 2. To a solution of tert-butyl 3-({2-[(tert-butyldimethylsilyl)oxy]ethyl}amino)propanoate (2.00 g, crude) and DIPEA (1.70 g, 13.2 mmol) in DCM (20 mL) was added acetyl chloride (0.80 g, 9.88 mmol) portionwise at 0° C. The resulting mixture was stirred for 3 h at room temperature and was then quenched by the addition of H₂O at 0° C. The aqueous layer was extracted with DCM, and the combined organic extracts were washed with brine (3×50 mL), dried over Na₂SO₄, filtered, and concentrated under reduced pressure. The crude material was purified by reversed phase chromatography to afford tert-butyl 3-(N-{2-[(tert-butyldimethylsilyl)oxy]ethyl}acetamido)propanoate (2.10 g, 53% yield over 2 steps) as a yellow oil. LCMS (ESI) m/z: [M+H] calcd for C₁₇H₃₅NO₄Si: 346.2; found 346.3.

Step 3. To a solution of tert-butyl 3-(N-{2-[(tert-butyldimethylsilyl)oxy]ethyl}acetamido)propanoate (3.00 g, 8.68 mmol) in DCM (24 mL) was added TFA (12 mL) dropwise at 0° C. The reaction mixture was stirred for 1 h at room temperature and was then neutralized to pH 7 by the addition of sat. aq. NaHCO₃. The aqueous layer was extracted with DCM and the combined organic extracts were washed with brine, dried over Na₂SO₄, filtered, and concentrated under reduced pressure to afford 3-[N-(2-hydroxyethyl)acetamido]propanoic acid (2.2 g, crude), which was used without further purification. LCMS (ESI) m/z: [M+H] calcd for C₇H₁₃NO₄: 176.1; found 176.3.

Step 4. To a solution of tert-butyl ((6³S,4S)-2⁵-(benzyloxy)-1¹-ethyl-1²-(2-((S)-1-methoxyethyl)pyridin-3-yl)-10,10-dimethyl-5,7-dioxo-6¹,6²,6³,6⁴,6⁵,6⁶-hexahydro-11H-8-oxa-1(5,3)-indola-6(1,3)-pyridazina-2(1,3)-benzenacycloundecaphane-4-yl)carbamate (2.70 g, 3.25 mmol) in MeOH (27 mL) was added 10% Pd/C (70 mg) at room temperature under an atmosphere of N₂. The reaction mixture was stirred overnight at room temperature under an atmosphere of H₂. The resulting mixture was then filtered, the filter cake washed with 10:1 vol. DCM:MeOH, and the filtrate concentrated under reduced pressure to afford tert-butyl ((6³S,4S)-1¹-ethyl-2⁵-hydroxy-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)carbamate (2.40 g, crude), which was used without further purification. LCMS (ESI) m/z: [M+H] calcd for C₄₂H₅₃N₅O₇: 740.4; found 740.7.

Step 5. To a solution of tert-butyl ((6³S,4S)-1¹-ethyl-2⁵-hydroxy-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)carbamate (1.50 g, crude) in 1,4-dioxane (8 mL) was added HCl (8 mL, 4 M solution in 1,4-dioxane) at 0° C. The resulting mixture was stirred for 2 h at room temperature and was then neutralized to pH 8 by the addition of sat. aq. NaHCO₃. The aqueous mixture was extracted with EtOAc, and the combined organic extracts were washed with brine, dried over Na₂SO₄, filtered, and concentrated under reduced pressure to afford (6³S,4S)-4-amino-1′-ethyl-2⁵-hydroxy-1²-(2-((S)-1-methoxyethyl)pyridin-3-yl)-10,10-dimethyl-6¹,6²,6³,6⁴,6⁵,6⁶-hexahydro-1¹H-8-oxa-1(5,3)-indola-6(1,3)-pyridazina-2(1,3)-benzenacycloundecaphane-5,7-dione (1.00 g, crude), which was used without further purification. LCMS (ESI) m/z: [M+H] calcd for C₃₇H₄₅N₅O₄: 640.3; found 640.3.

Step 6. To a solution of (6³S,4S)-4-amino-1′-ethyl-2⁵-hydroxy-1²-(2-((S)-1-methoxyethyl)pyridin-3-yl)-10,10-dimethyl-6¹,6²,6³,6⁴,6⁵,6⁶-hexahydro-1¹H-8-oxa-1(5,3)-indola-6(1,3)-pyridazina-2(1,3)-benzenacycloundecaphane- 5,7-dione (1.00 g, crude) and DIPEA (8.08 g, 62.5 mmol) in DMF (10 mL) were added (2S)-2-[(tert-butoxycarbonyl)(methyl)amino]-3-methylbutanoic acid (0.54 g, 2.35 mmol) and COMU (80.0 g, 1.88 mmol) at 0° C. The resulting mixture was stirred for 2 h at room temperature under nitrogen atmosphere and was quenched by the addition of H₂O. The aqueous mixture was extracted with EtOAc, and the combined organic extracts were washed with brine, dried over Na₂SO₄, filtered, and concentrated under reduced pressure. The crude material was purified by prep-TLC to afford tert-butyl ((2S)-1-(((6³S,4S)-1¹-ethyl-2⁵-hydroxy-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)amino)-3-methyl-1-oxobutan-2-yl)(methyl)carbamate (800 mg, 46% yield over 3 steps) as a yellow solid. LCMS (ESI) m/z: [M+H] calcd for C₄₈H₆₄N₆O₈: 853.5; found 853.6.

Step 7. To a solution of tert-butyl ((2S)-1-(((6³S,4S)-1¹-ethyl-2⁵-hydroxy-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)amino)-3-methyl-1-oxobutan-2-yl)(methyl)carbamate (380 mg, 0.445 mmol) in 1,4-dioxane (2 mL) was added HCl (2 mL, 4 M solution in 1,4-dioxane) at 0° C. The resulting mixture was stirred for 2 h at room temperature and was then concentrated under reduced pressure. The residue was neutralized to pH 8 by the addition of sat. aq. NaHCO₃. The aqueous mixture was extracted with EtOAc (5×100 mL) and the combined organic extracts were washed with brine (3×50 mL), dried over Na₂SO₄, filtered, and concentrated under reduced pressure to afford (2S)—N-((6³S,4S)-1¹-ethyl-2⁵-hydroxy-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)- benzenacycloundecaphane-4-yl)-3-methyl-2-(methylamino)butanamide (250 mg, crude), which was used without further purification. LCMS (ESI) m/z: [M+H] calcd for C₄₃H₅₆N₆O₄: 753.4; found 753.3.

Step 8. To a solution of (2S)—N-((6³S,4S)-1¹-ethyl-2⁵-hydroxy-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)- benzenacycloundecaphane-4-yl)-3-methyl-2-(methylamino)butanamide (200 mg, crude) and DIPEA (137 g, 10.6 mmol) in DMF (2 mL) were added 3-[N-(2-hydroxyethyl)acetamido]propanoic acid (69.8 mg, crude) and HATU (151 mg, 0.399 mmol) dropwise at 0° C. The resulting mixture was stirred for 2 h at room temperature and was then quenched by the addition of H₂O. The aqueous mixture was extracted with EtOAc, and the combined organic extracts were washed with brine, dried over Na₂SO₄, filtered, and concentrated under reduced pressure. The crude residue was purified by prep-TLC to afford (2S)—N-((6³S,4S)-1¹-ethyl-2⁵-hydroxy-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)-2-(3-(N-(2-hydroxyethyl)acetamido)-N-methylpropanamido)-3-methylbutanamide (60.0 mg, 19% yield over 2 steps) as a yellow solid. LCMS (ESI) m/z: [M+H] calcd for C₅₀H₆₇N₇O₉: 910.5; found 910.8.

Step 9. To a solution of (2S)—N-((6³S,4S)-1¹-ethyl-2⁵-hydroxy-1²-(2-((S)-1-methoxyethyl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)- benzenacycloundecaphane-4-yl)-2-(3-(N-(2-hydroxyethyl)acetamido)-N-methylpropanamido)-3-methylbutanamide (35.0 mg, 0.038 mmol) in toluene (20 mL) was added CMBP (46.4 mg, 0.190 mmol) at 0° C. The resulting mixture was stirred for at room temperature for 2 hours and was then stirred overnight at 60° C. before being quenched by the addition of H₂O. The aqueous mixture was extracted with EtOAc, and the combined organic extracts were washed with brine, dried over Na₂SO₄, filtered, and concentrated under reduced pressure. The crude product was purified by reversed phase chromatography to afford (9S,15S,18S)-23-acetyl-2-ethyl-18-isopropyl-3-(2-((S)-1-methoxyethyl)pyridin-3-yl)-5,5,19-trimethyl-2,4,5,6,9,10,11,12,15,16,18,19,22,23,24,25-hexadecahydro-8H-9,13-epimino-1,32-etheno-15,29-methano-27,31-(metheno)pyrrolo[3,4-x][1,20]dioxa[4,8,11,14]tetraazacyclodotriacontine-8,14,17,20(21H)-tetraone (1.1 mg, 3.0% yield) as a white solid. LCMS (ESI) m/z: [M+H] calcd for C₅₀H₆₅N₇O₈: 892.5; found 892.8. ¹H NMR (400 MHz, DMSO-d₆) δ 8.76 (dd, J=4.8, 1.7 Hz, 1H), 8.61-8.38 (m, 1H), 8.02 (d, J=5.4 Hz, 1H), 7.89-7.72 (m, 2H), 7.60-7.48 (m, 2H), 7.35 (s, 1H), 7.28-7.14 (m, 1H), 6.77-6.72 (m, 1H), 5.35-5.10 (m, 2H), 4.80 (d, J=11.0 Hz, 1H), 4.37-3.76 (m, 10H), 3.70-3.58 (m, 2H), 3.55-3.40 (m, 5H), 3.10 (d, J=5.9 Hz, 3H), 3.00-2.70 (m, 7H), 2.70-2.61 (m, 1H), 2.21-1.95 (m, 6H), 1.85-1.61 (m, 2H), 1.59-1.51 (m, 1H), 1.41-1.34 (m, 4H), 1.24 (br s, 1H), 1.11-0.62 (m, 14H), 0.61-0.33 (m, 3H).

### Example A62. Synthesis of (9S,13S,16S,19S,25S)-33-ethyl-16-isopropyl-10-(3-methoxyazetidine-1-carbonyl)-32-[2-[(1S)-1-methoxyethyl]-3-pyridyl]-15,29,29-trimethyl-7,27-dioxa-10,15,18,21,33,39-hexazaheptacyclo[29.5.2.1^{2,6}.1^{4,19}.1^{21,25}.0^{9,13}.0^{34,38}]hentetraconta-1(37),2(41),3,5,31,34(38),35-heptaene-14,17,20,26-tetraone

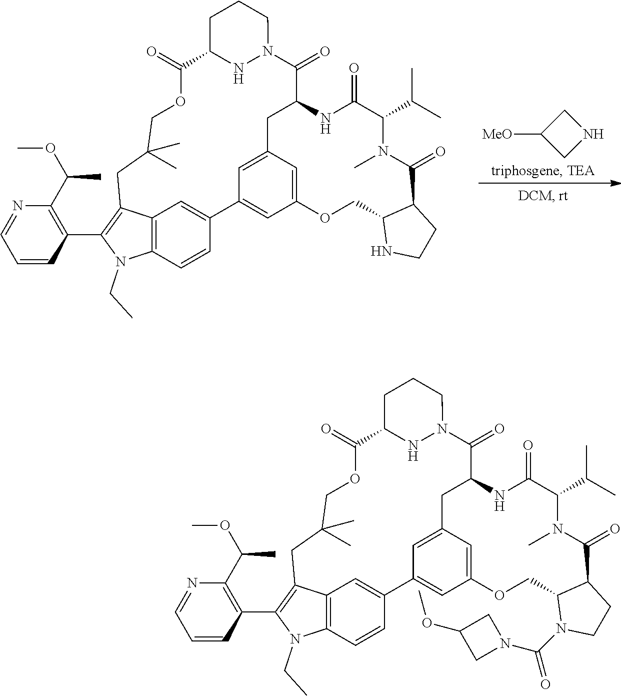

Step 1. To a solution of (9S,13S,16S,19S,25S)-33-ethyl-16-isopropyl-32-[2-[(1 S)-1-methoxyethyl]-3-pyridyl]-15,29,29-trimethyl-7,27-dioxa-10,15,18,21,33,39-hexazaheptacyclo[29.5.2.1^{2,6}.1^{4,19}.1^{21,25}.0^{9,13}.0^{34,38}]hentetraconta-1(37),2(41),3,5,31,34(38),35-heptaene-14,17,20,26-tetarone (60.0 mg, 0.696 mmol) in DCM (1 mL) at 0° C. were added DIPEA (90.0 mg, 0.696 mmol) followed by triphosgene (8.26 mg, 0.028 mmol). The resulting mixture was stirred at room temperature for 1 h, 3-methoxyazetidine hydrochloride (7.74 mg, 0.063 mmol) was subsequently added at 0° C., and the resulting mixture was stirred at room temperature for 2 h. The mixture was then filtered, concentrated under reduced pressure, and the resulting crude material was purified by reversed phase chromatography to give (9S,13S,16S,19S,25S)-33-ethyl-16-isopropyl-10-(3-methoxyazetidine-1-carbonyl)-32-[2-[(1 S)-1-methoxyethyl]-3-pyridyl]-15,29,29-trimethyl-7,27-dioxa-10,15,18,21,33,39-hexazaheptacyclo[29.5.2.1^{2,6}.1^{4,19}.1^{21,25}.0^{9,13}.0^{34,38}]hentetraconta-1(37),2(41),3,5,31,34(38),35-heptaene-14,17,20,26-tetraone (40 mg, 57% yield) as a white solid. LCMS (ESI) m/z: [M+H] calcd for C₅₄H₇₀N₈O₉: 975.5; found 975.9; ¹H NMR (400 MHz, DMSO-d₆) δ=8.76 (d, J=4.0 Hz, 1H), 8.23 (d, J=8.0 Hz, 1H), 8.02 (s, 1H), 7.86-7.79 (m, 1H), 7.76-7.70 (m, 1H), 7.61-7.44 (m, 2H), 7.42-7.31 (m, 1H), 7.27-7.13 (m, 1H), 6.83-6.72 (m, 1H), 5.37-5.08 (m, 2H), 4.72 (d, J=12.0 Hz, 1H), 4.44-4.17 (m, 7H), 4.15-3.76 (m, 5H), 3.72-3.42 (m, 5H), 3.23 (s, 3H), 3.15-3.09 (m, 3H), 2.90-2.71 (m, 4H), 2.4 (s, 3H), 2.22-2.11 (m, 1H), 2.08-1.95 (m, 2H), 1.93-1.79 (m, 1H), 1.77-1.49 (m, 3H), 1.46-1.36 (m, 3H), 1.30-1.12 (m, 1H), 1.00-0.64 (m, 12H), 0.50-0.44 (m, 3H).

### Example A80. Synthesis of (3aS,6S,9S,15S,32aS)-21-(5-(4-cyclopropylpiperazin-1-yl)-2-((S)-1-methoxyethyl)pyridin-3-yl)-22-ethyl-6-isopropyl-5,19,19-trimethyl-1-propionyl-3,3a,5,6,8,9,14,15,18,19,20,22,32,32a-tetradecahydro-1H,12H-11,15-epimino-23,25-etheno-9,28-methano-26,30-(metheno)dipyrrolo[2,3-c:3′,4′-v][1,18]dioxa[6,9,12]triazacyclotriacontine-4,7,10,16(2H,13H)-tetraone

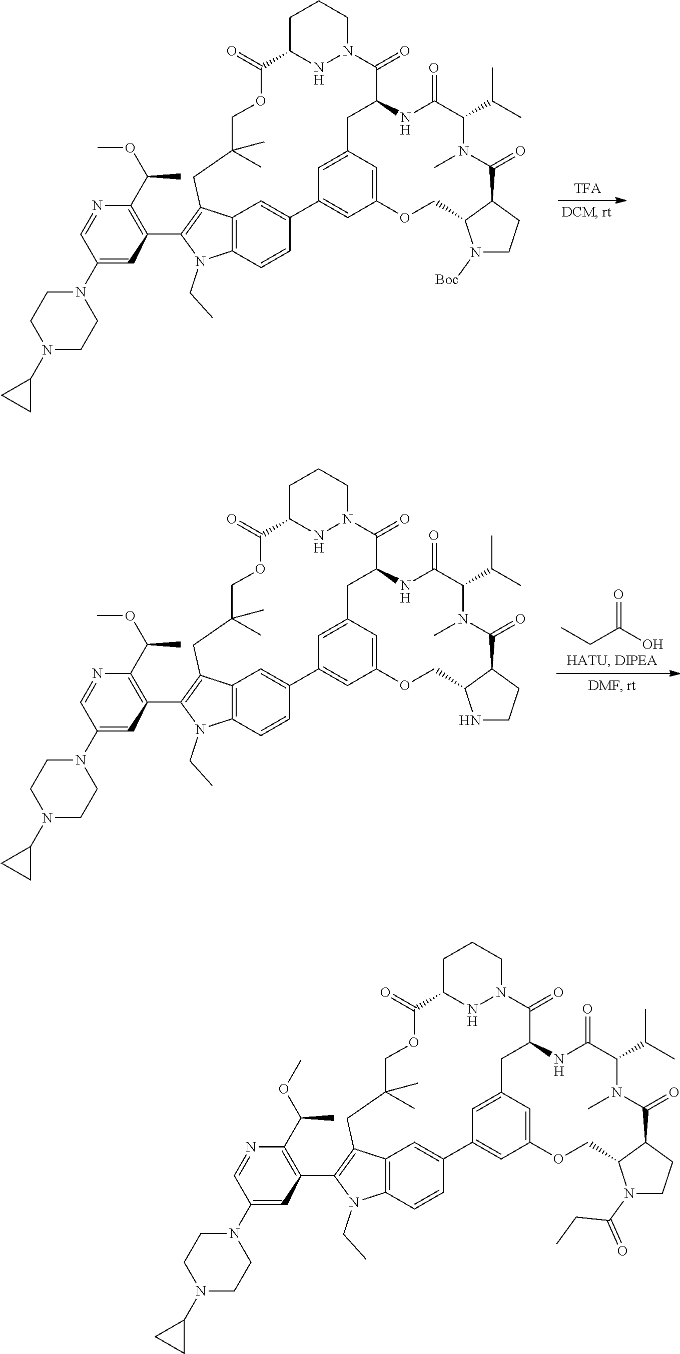

Step 1. To a stirred solution of tert-butyl (3aS,6S,9S,15S,32aS)-21-(5-(4-cyclopropylpiperazin-1-yl)-2-((S)-1-methoxyethyl)pyridin-3-yl)-22-ethyl-6-isopropyl-5,19,19-trimethyl-4,7,10,16-tetraoxo-2,3,3a,4,5,6,7,8,9,10,13,14,15,16,18,19,20,22,32,32a-icosahydro-1H,12H-11,15-epimino-23,25-etheno-9,28-methano-26,30-(metheno)dipyrrolo[2,3-c:3′,4′-v][1,18]dioxa[6,9,12]triazacyclotriacontine-1-carboxylate (4.70 g, 4.33 mmol) in DCM (20 mL) was added TFA (4.00 mL, 35.1 mmol) dropwise at 0° C. The reaction mixture was stirred for 1 h at room temperature and was then concentrated under reduced pressure to afford (3aS,6S,9S,15S,32aS)-21-(5-(4-cyclopropylpiperazin-1-yl)-2-((S)-1-methoxyethyl)pyridin-3-yl)-22-ethyl-6-isopropyl-5,19,19-trimethyl-3,3a,5,6,8,9,14,15,18,19,20,22,32,32a-tetradecahydro-1H,12H-11,15-epimino-23,25-etheno-9,28-methano-26,30-(metheno)dipyrrolo[2,3-c:3′,4′-v][1,18]dioxa[6,9,12]triazacyclotriacontine-4,7,10,16(2H,13H)-tetraone (5.96 g, crude). LCMS (ESI) m/z: [M+H] calcd for C₅₆H₇₅N₉O₇: 986.6; found 986.5.

Step 2. To a stirred solution of (3aS,6S,9S,15S,32aS)-21-(5-(4-cyclopropylpiperazin-1-yl)-2-((S)-1-methoxyethyl)pyridin-3-yl)-22-ethyl-6-isopropyl-5,19,19-trimethyl-3,3a,5,6,8,9,14,15,18,19,20,22,32,32a-tetradecahydro-1H,12H-11,15-epimino-23,25-etheno-9,28-methano-26,30-(metheno)dipyrrolo[2,3-c:3′,4′-v][1,18]dioxa[6,9,12]triazacyclotriacontine-4,7,10,16(2H,13H)-tetraone (310 mg, crude) in DMF were added DIPEA (406 mg, 3.14 mmol), propanoic acid (46.6 mg, 0.628 mmol), and HATU (239 mg, 0.628 mmol). The reaction mixture was stirred for 30 min. at 0° C. and was then quenched by the addition of H₂O (40 mL) at 0° C. The resulting mixture was extracted with EtOAc (3×10 mL) and concentrated under reduced pressure. The residue was purified by reversed phase prep-HPLC to give (3aS,6S,9S,15S,32aS)-21-(5-(4-cyclopropylpiperazin-1-yl)-2-((S)-1-methoxyethyl)pyridin-3-yl)-22-ethyl-6-isopropyl-5,19,19-trimethyl-1-propionyl-3,3a,5,6,8,9,14,15,18,19,20,22,32,32a-tetradecahydro-1H,12H-11,15-epimino-23,25-etheno-9,28-methano-26,30-(metheno)dipyrrolo[2,3-c:3′,4′-v][1,18]dioxa[6,9,12]triazacyclotriacontine-4,7,10,16(2H,13H)-tetraone (99 mg, 42% yield over 2 steps) as a white solid. LCMS (ESI) m/z: [M+H] calcd for C₅₉H₇₉N₉O₈: 1042.6; found 1042.5. ¹H NMR (400 MHz, DMSO-d₆) δ 8.48 (s, 1H), 8.20-7.93 (m, 1H), 7.75-7.70 (m, 1H), 7.61-7.54 (m, 1H), 7.50-7.40 (m, 1H), 7.39-7.12 (m, 2H), 6.83-6.05 (m, 2H), 5.46-5.00 (m, 2H), 4.71-4.51 (m, 1H), 4.47 (s, 1H), 4.45-4.30 (m, 1H), 4.33-3.88 (m, 5H), 3.90-3.60 (m, 4H), 3.23 (s, 3H), 3.10-2.95 (m, 5H), 2.90-2.71 (m, 7H), 2.69-2.60 (m, 1H), 2.39 (s, 3H), 2.32-2.16 (m, 4H), 2.10-1.90 (m, 3H), 1.90-1.79 (m, 2H), 1.77-1.55 (m, 4H), 1.50-1.15 (m, 6H), 1.10-0.90 (m, 6H), 0.91-0.43 (m, 16H).

### Example A126. Synthesis of (3aS,6S,9S,15S,32aS)-22-ethyl-6-isopropyl-21-(2-((S)-1-methoxyethyl)-5-(4-methylpiperazin-1-yl)pyridin-3-yl)-5,19,19-trimethyl-1-(thiazol-2-yl)-3,3a,5,6,8,9,14,15,18,19,20,22,32,32a-tetradecahydro-1H,12H-11,15-epimino-23,25-etheno-9,28-methano-26,30-(metheno)dipyrrolo[2,3-c:3′,4′-v][1,18]dioxa[6,9,12]triazacyclotriacontine-4,7,10,16(2H,13H)-tetraone

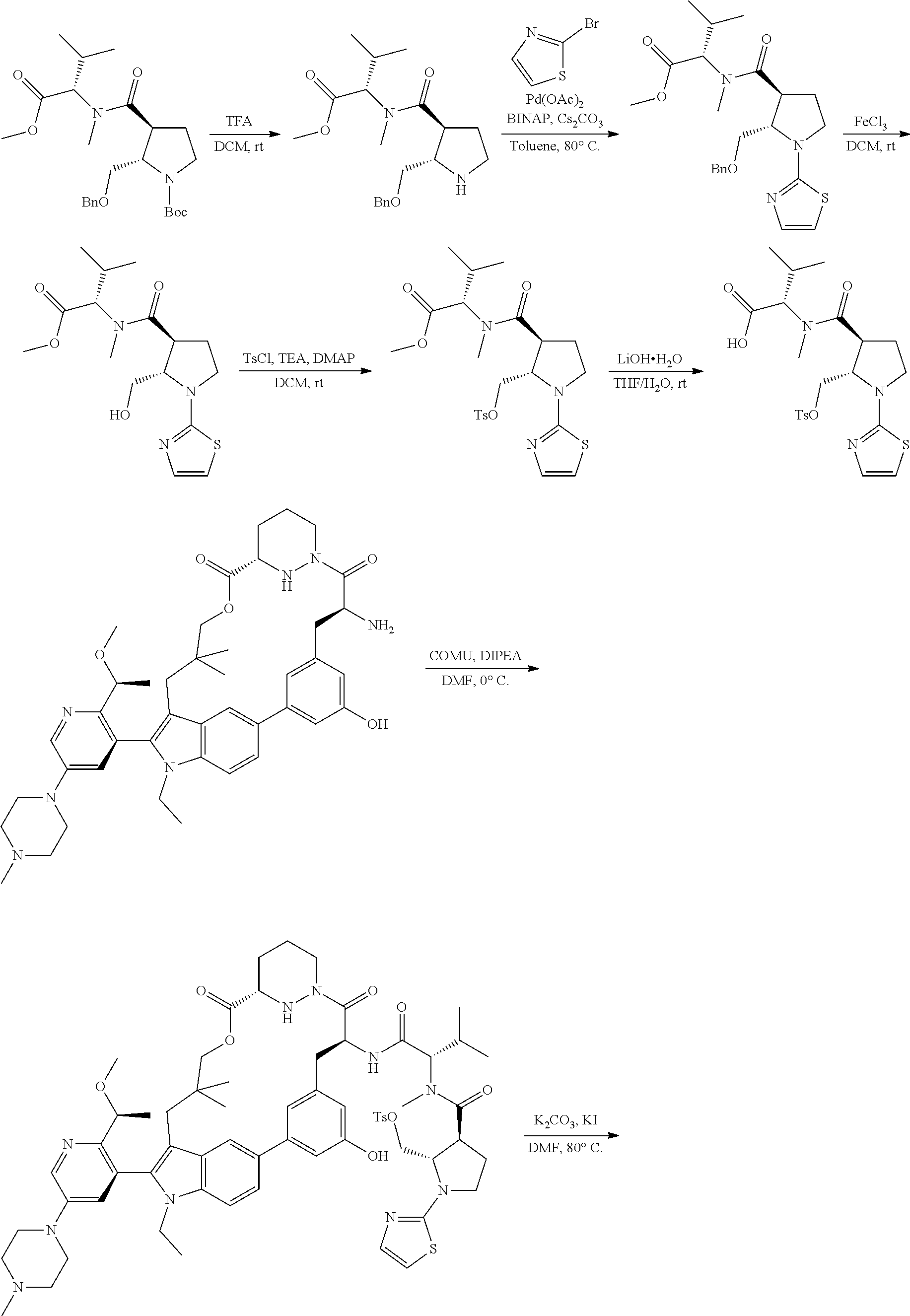

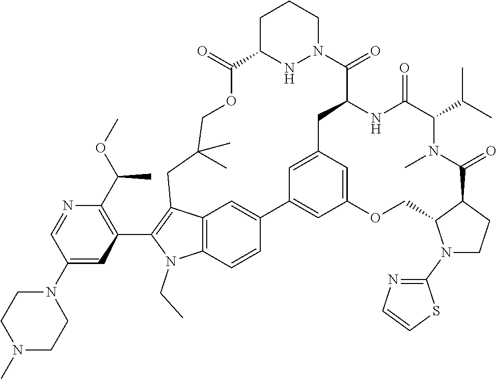

Step 1. To a solution of tert-butyl (2S,3S)-2-((benzyloxy)methyl)-3-(((S)-1-methoxy-3-methyl-1-oxobutan-2-yl)(methyl)carbamoyl)pyrrolidine-1-carboxylate (3.0 g, 6.5 mmol) in DCM (24 mL) at 0° C. was added TFA (8.0 mL). The resulting mixture was stirred overnight at room temperature and was then concentrated under reduced pressure. The residue was dissolved in DCM (10 mL) and resulting solution was basified to pH 8 with sat. aq. NaHCO₃. The aqueous layer was extracted with DCM (3×10 mL) and the combined organic extracts were dried over Na₂SO₄, filtered, and concentrated under reduced pressure to afford N-methyl-N-((2S,3S)-1-(thiazol-2-yl)-2-((tosyloxy)methyl)pyrrolidine-3-carbonyl)-L-valinate (2.86 g, crude), which was used without further purification. LCMS (ESI) m/z: [M+H] calcd for C₂₀H₃₀N₂O₄: 363.2; found 362.9.

Step 2. To a solution of methyl N-((2S,3S)-2-((benzyloxy)methyl)pyrrolidine-3-carbonyl)-N-methyl-L-valinate (2.87 g, 7.91 mmol) and 2-bromothiazole (3.89 g, 23.7 mmol) in toluene (30 mL) was added Cs₂CO₃ (7.74 g, 23.7 mmol), Pd(OAc)₂ (1.07 g, 4.75 mmol) and BINAP (5.91 g, 9.45 mmol). The resulting mixture was stirred overnight at 80° C. and the reaction was then quenched at 0° C. by the addition of cold H₂O. The aqueous layer was extracted with EtOAc (3×10 mL), and the combined organic extracts were dried over Na₂SO₄, filtered, and concentrated under reduced pressure. Purification by reversed phase chromatography afforded methyl N-((2S,3S)-2-((benzyloxy)methyl)-1-(thiazol-2-yl)pyrrolidine-3-carbonyl)-N-methyl-L-valinate (923 mg, 26% yield) as a red oil. LCMS (ESI) m/z: [M+H] calcd for C₂₃H₃₁N₃O₄S: 446.2; found 446.2.

Step 3. To a solution of methyl N-((2S,3S)-2-((benzyloxy)methyl)-1-(thiazol-2-yl)pyrrolidine-3-carbonyl)-N-methyl-L-valinate (821 mg, 1.84 mmol) in DCM (4.0 mL) at 0° C. was added FeCl₃ (1.20 g, 7.38 mmol). The resulting mixture was stirred overnight at room temperature and the reaction was then quenched at 0° C. by the addition of sat. aq. NaHCO₃. The mixture was filtered, and the aqueous layer extracted with DCM (3×5 mL). The filter cake was washed with DCM (3×5 mL) followed by MeOH (3×10 mL) and the combined organic extracts were concentrated under reduced pressure to afford methyl N-((2S,3S)-2-(hydroxymethyl)-1-(thiazol-2-yl)pyrrolidine-3-carbonyl)-N-methyl-L-valinate (934 mg, crude) which was used without further purification. LCMS (ESI) m/z: [M+H] calcd for C₁₅H₂₅N₃O₄S: 356.2; found 356.2.

Step 4. To a solution of methyl N-((2S,3S)-2-(hydroxymethyl)-1-(thiazol-2-yl)pyrrolidine-3-carbonyl)-N-methyl-L-valinate (933 mg, crude) and Et₃N (1.06 g, 10.5 mmol) in DCM (4.0 mL) at 0° C. were added TsCl (1.50 g, 7.88 mmol) and DMAP (32 mg, 0.26 mmol). The resulting mixture was stirred for 2 h at room temperature and the reaction was then quenched at 0° C. by the addition of cold H₂O. The aqueous layer was extracted with DCM (3×10 mL) and the combined organic extracts were dried over Na₂SO₄, filtered, and concentrated under reduced pressure. Purification by normal phase chromatography afforded methyl N-methyl-N-((2S,3S)-1-(thiazol-2-yl)-2-((tosyloxy)methyl)pyrrolidine-3-carbonyl)-L-valinate (644 mg, 67% yield) as a brown oil. LCMS (ESI) m/z: [M+H] calcd for C₂₃H₃₁N₃O₆S₂: 510.2; found 510.2.

Step 5. To a solution of methyl N-methyl-N-((2S,3S)-1-(thiazol-2-yl)-2-((tosyloxy)methyl)pyrrolidine-3-carbonyl)-L-valinate (320 mg, 0.628 mmol) in THE (5.0 ml) at 0° C. were added LiOH·H₂O (79 mg, 1.8 mmol) and H₂O (1.0 mL). The resulting mixture was stirred overnight at room temperature and the reaction solution was acidified to pH 6 with 2 M aq. HCl. The aqueous mixture was extracted with DCM (3×20 mL) and the combined organic extracts were dried over Na₂SO₄, filtered, and concentrated under reduced pressure to afford N-methyl-N-((2S,3S)-1-(thiazol-2-yl)-2-((tosyloxy)methyl)pyrrolidine-3-carbonyl)-L-valine (355 mg, crude) which was used without further purification. LCMS (ESI) m/z: [M+H] calcd for C₂₂H₂₉N₃O₆S₂: 496.2; found 496.1.

Step 6. To a solution of (6³S,4S)-4-amino-1′-ethyl-2⁵-hydroxy-1²-(2-((S)-1-methoxyethyl)-5-(4-methylpiperazin-1-yl)pyridin-3-yl)-10,10-dimethyl-6¹,6²,6³,6⁴,6⁵,6⁶-hexahydro-1¹H-8-oxa-1(5,3)-indola-6(1,3)-pyridazina-2(1,3)-benzenacycloundecaphane-5,7-dione (264 mg, 0.358 mmol) and N-methyl-N-((2S,3S)-1-(thiazol-2-yl)-2-((tosyloxy)methyl)pyrrolidine-3-carbonyl)-L-valine (177 mg, crude) in DMF (4.0 mL) at 0° C. were added DIPEA (1.85 g, 14.3 mmol) and COMU (230 mg, 0.537 mmol). The resulting mixture was stirred for 1.5 h at 0° C. and the reaction was then quenched at 0° C. by the addition of cold H₂O. The aqueous mixture was extracted with EtOAc (3×10 mL), and the combined organic extracts were washed with brine, dried over Na₂SO₄, filtered, and concentrated under reduced pressure.

Purification by reversed phase chromatography afforded ((2S,3S)-3-(((2S)-1-(((6³S,4S)-1¹-ethyl-2⁵-hydroxy-1²-(2-((S)-1-methoxyethyl)-5-(4-methylpiperazin-1-yl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)amino)-3-methyl-1-oxobutan-2-yl)(methyl)carbamoyl)-1-(thiazol-2-yl)pyrrolidin-2-yl)methyl 4-methylbenzenesulfonate (187 mg, 43% yield) as a yellow solid. LCMS (ESI) m/z: [M+H] calcd for C₆₄H₈₂N₁₀O₁₀S₂: 1215.6; found 1215.2.

Step 7. To a solution of ((2S,3S)-3-(((2S)-1-(((6³S,4S)-1¹-ethyl-2⁵-hydroxy-1²-(2-((S)-1-methoxyethyl)-5-(4-methylpiperazin-1-yl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)amino)-3-methyl-1-oxobutan-2-yl)(methyl)carbamoyl)-1-(thiazol-2-yl)pyrrolidin-2-yl)methyl 4-methylbenzenesulfonate (185 mg, 0.152 mmol) in DMF (18.5 mL) were added K₂CO₃ (210 mg, 1.52 mmol) and KI (25.3 mg, 0.152 mmol). The resulting mixture was stirred for 2 h at 80° C. and the reaction was then quenched at −78° C. with H₂O. The aqueous mixture was extracted EtOAc (3×20 mL), and the combined organic extracts were washed with brine, dried over Na₂SO₄, filtered, and concentrated under reduced pressure. Purification by reversed phase chromatography afforded (3aS,6S,9S,15S,32aS)-22-ethyl-6-isopropyl-21-(2-((S)-1-methoxyethyl)-5-(4-methylpiperazin-1-yl)pyridin-3-yl)-5,19,19-trimethyl-1-(thiazol-2-yl)-3,3a,5,6,8,9,14,15,18,19,20,22,32,32a-tetradecahydro-1H,12H-11,15-epimino-23,25-etheno-9,28-methano-26,30-(metheno)dipyrrolo[2,3-c:3′,4′-v][1,18]dioxa[6,9,12]triazacyclotriacontine-4,7,10,16(2H,13H)-tetraone (24.2 mg, 15% yield) as a white solid. LCMS (ESI) m/z: [M+H] calcd for C₅₇H₇₄N₁₀O₇S: 1043.6; found 1043.4. ¹H NMR (400 MHz, DMSO-d₆) δ 8.45 (d, J=2.6 Hz, 1H), 8.39-8.17 (m, 1H), 8.11-7.95 (m, 1H), 7.83-7.66 (m, 1H), 7.64-7.50 (m, 2H), 7.49-7.36 (m, 1H), 7.34-7.11 (m, 2H), 7.08-6.52 (m, 2H), 5.46-5.06 (m, 2H), 4.83-4.66 (m, 1H), 4.64-4.46 (m, 1H), 4.44-3.80 (m, 7H), 3.77-3.60 (m, 5H), 3.35-3.11 (m, 5H), 3.09-2.95 (m, 2H), 2.91-2.57 (m, 5H), 2.53-2.35 (m, 6H), 2.33-2.12 (m, 4H), 1.92-1.51 (m, 6H), 1.45-1.15 (m, 4H), 1.09-0.93 (m, 2H), 7.83-7.66 (m, 1H), 0.91-0.51 (m, 12H), 0.49-0.37 (m, 1H).

### Example A130. Synthesis of (3aS,6S,9S,15S,32aS)-21-(5-(4-cyclopropylpiperazin-1-yl)-2-((S)-1-methoxyethyl)pyridin-3-yl)-22-ethyl-6-isopropyl-5,19,19-trimethyl-1-(oxetan-3-ylmethyl)-3,3a,5,6,8,9,14,15,18,19,20,22,32,32a-tetradecahydro-1H,12H-11,15-epimino-23,25-etheno-9,28-methano-26,30-(metheno)dipyrrolo[2,3-c:3′,4′-v][1,18]dioxa[6,9,12]triazacyclotriacontine-4,7,10,16(2H,13H)-tetraone

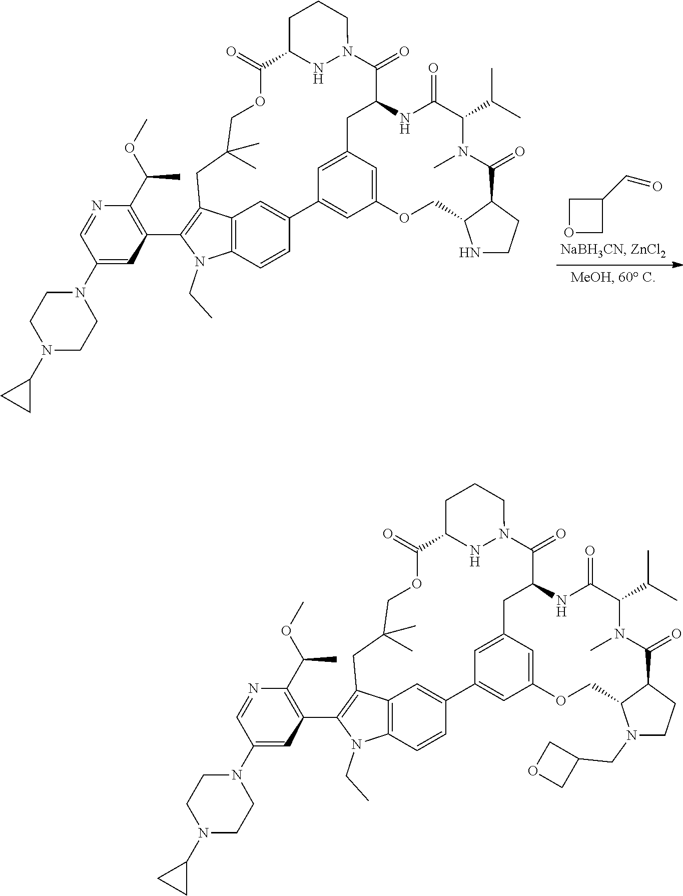

Step 1. To a stirred solution of (3aS,6S,9S,15S,32aS)-21-(5-(4-cyclopropylpiperazin-1-yl)-2-((S)-1-methoxyethyl)pyridin-3-yl)-22-ethyl-6-isopropyl-5,19,19-trimethyl-3,3a,5,6,8,9,14,15,18,19,20,22,32,32a-tetradecahydro-1H,12H-11,15-epimino-23,25-etheno-9,28-methano-26,30-(metheno)dipyrrolo[2,3-c:3′,4′-v][1,18]dioxa[6,9,12]triazacyclotriacontine-4,7,10,16(2H,13H)-tetraone (200 mg, 0.203 mmol) and oxetane-3-carbaldehyde (69.8 mg, 0.812 mmol) in MeOH (5 mL) at 0 C was added NaBH₃CN (51 mg, 0.812 mmol) and ZnCl₂ (111 mg, 0.812 mmol) then the mixture was heated to 60° C. After 1 h, the reaction was cooled to 0° C., quenched with water (5 mL), concentrated under reduced pressure, basified to pH 8 with sat. aq. NaHCO₃, extracted into EtOAc (3×10 mL), dried over Na₂SO₄, filtered, concentrated under reduced pressure. The resulting residue was purified by reversed phase column chromatography to afford (3aS,6S,9S,15S,32aS)-21-(5-(4-cyclopropylpiperazin-1-yl)-2-((S)-1-methoxyethyl)pyridin-3-yl)-22-ethyl-6-isopropyl-5,19,19-trimethyl-1-(oxetan-3-ylmethyl)-3,3a,5,6,8,9,14,15,18,19,20,22,32,32a-tetradecahydro-1H,12H-11,15-epimino-23,25-etheno-9,28-methano-26,30-(metheno)dipyrrolo[2,3-c:3′,4′-v][1,18]dioxa[6,9,12]triazacyclotriacontine-4,7,10,16(2H,13H)-tetraone (47.6 mg, 22% yield) as a light yellow solid. LCMS (ESI) m/z: [M+H] calcd for C₆₀H₈₁N₉O₈: 1056.6; found 1056.4. ¹H NMR (400 MHz, DMSO-d₆) δ 8.45 (s, 1H), 8.30-8.15 (m, 1H), 7.95 (s, 1H), 7.68 (d, J=8.8 Hz, 1H), 7.56 (d, J=8.7 Hz, 1H), 7.45-7.38 (m, 1H), 7.19 (s, 2H), 6.66 (s, 1H), 5.29-5.08 (m, 2H), 4.86-4.49 (m, 3H), 4.42-4.02 (m, 8H), 3.89-3.70 (m, 4H), 3.25-3.13 (m, 8H), 3.07-2.95 (s, 4H), 2.98-2.89 (m, 2H), 2.83-2.63 (m, 8H), 2.34-2.20 (m, 2H), 2.06 (s, 4H), 1.88-1.45 (m, 6H), 1.41-1.17 (m, 4H), 1.09-0.59 (m, 12H), 0.57-0.24 (m, 7H).

### Example A138. Synthesis of (3aS,6S,9S,15S,32aS)-21-(5-(4-cyclopropylpiperazin-1-yl)-2-((S)-1-methoxyethyl)pyridin-3-yl)-22-ethyl-6-isopropyl-5,19,19-trimethyl-1-(methylsulfonyl)-3,3a,5,6,8,9,14,15,18,19,20,22,32,32a-tetradecahydro-1H,12H-11,15-epimino-23,25-etheno-9,28-methano-26,30-(metheno)dipyrrolo[2,3-c:3′,4′-v][1,18]dioxa[6,9,12]triazacyclotriacontine-4,7,10,16(2H,13H)-tetraone

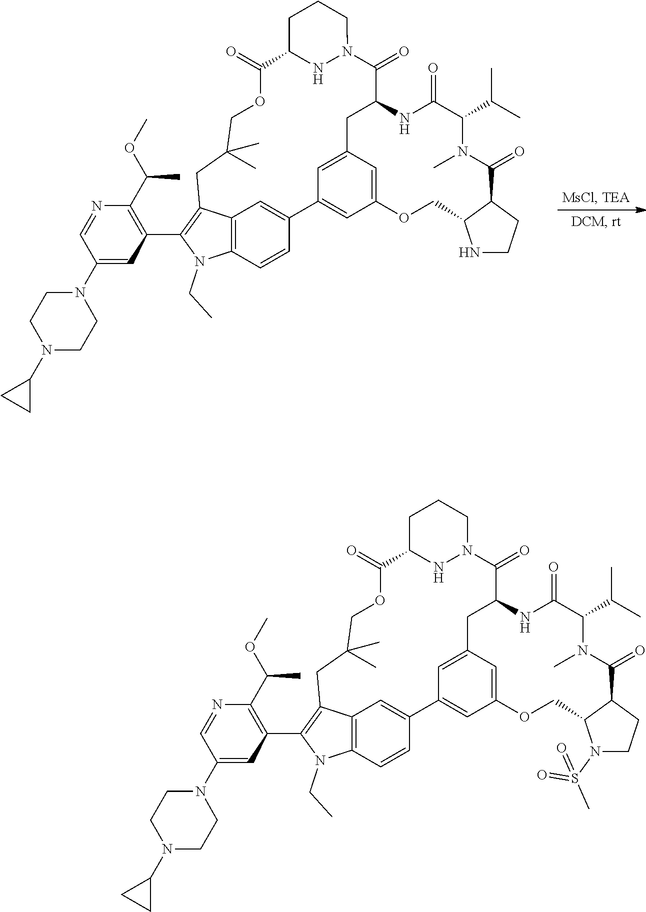

Step 1. To a stirred solution of tert-butyl (9S,13S,16S,19S,25S)-32-[5-(4-cyclopropylpiperazin-1-yl)-2-[(1 S)-1-methoxyethyl]-3-pyridyl]-33-ethyl-16-isopropyl-15,29,29-trimethyl-14,17,20,26-tetraoxo-7,27-dioxa-10,15,18,21,33,39-hexazaheptacyclo[29.5.2.1^{2,6}.1^{4,19}.1^{21,25}.0^{9,13}.0^{34,38}]hentetraconta-1(37),2(41),3,5,31,34(38),35-heptaene-10-carboxylate (100 mg, 0.101 mmol) in DCM (4 mL) were added TEA (103 mg, 1.01 mmol) and MsCl (23.2 mg, 0.202 mmol) at 0° C. The reaction mixture was stirred for 30 minutes at room temperature and was then quenched by the addition of H₂O (10 mL) at 0° C. The aqueous phase was extracted with DCM (3×10 mL) and the combined organic extracts were combined, dried over Na₂SO₄, filtered, and concentrated under reduced pressure. The residue was then purified by reversed phase chromatography to give (3aS,6S,9S,15S,32aS)-21-(5-(4-cyclopropylpiperazin-1-yl)-2-((S)-1-methoxyethyl)pyridin-3-yl)-22-ethyl-6-isopropyl-5,19,19-trimethyl-1-(methylsulfonyl)-3,3a,5,6,8,9,14,15,18,19,20,22,32,32a-tetradecahydro-1H,12H-11,15-epimino-23,25-etheno-9,28-methano-26,30-(metheno)dipyrrolo[2,3-c:3′,4′-v][1,18]dioxa[6,9,12]triazacyclotriacontine-4,7,10,16(2H,13H)-tetraone (12.8 mg, 12% yield) as a white solid LCMS (ESI) m/z: [M+H] calcd for C₅₇H₇₇N₉O₉S: 1064.6; found 1064.4. ¹H NMR (300 MHz, DMSO-d₆) δ 8.46 (s, 1H), 8.27 (d, J=8.0 Hz, 1H), 8.00 (s, 1H), 7.73 (d, J=8.4 Hz, 1H), 7.64-7.49 (m, 1H), 7.43 (d, J=12.3 Hz, 1H), 7.35-7.09 (m, 2H), 6.81-6.53 (m, 1H), 5.33 (d, J=12.0 Hz, 1H), 4.84-3.98 (m, 7H), 3.69 (s, 4H), 3.23 (s, 6H), 3.06 (d, J=5.7 Hz, 5H), 2.88 (d, J=14.2 Hz, 4H), 2.70 (d, J=11.9 Hz, 7H), 2.23 (d, J=23.9 Hz, 1H), 2.03 (d, J=11.9 Hz, 2H), 1.66-1.82 (m, 5H), 1.45-1.17 (m, 5H), 1.00 (d, J=7.0 Hz, 2H), 0.95-0.65 (m, 1OH), 0.64-0.20 (m, 8H).

### Example A140. Synthesis of N-((9S,15S,18S,22S)-3-(5-(4-cyclopropylpiperazin-1-yl)-2-((S)-1-methoxyethyl)pyridin-3-yl)-2-ethyl-18-isopropyl-5,5,19-trimethyl-8,14,17,20-tetraoxo-2,4,5,6,9,10,11,12,14,15,16,17,18,19,20,21,22,23-octadecahydro-8H-9,13-epimino-1,30-etheno-15,27-methano-25,29-(metheno)pyrrolo[3,4-v][1,18]dioxa[6,9,12]triazacyclotriacontin-22-yl)-N-methylacetamide

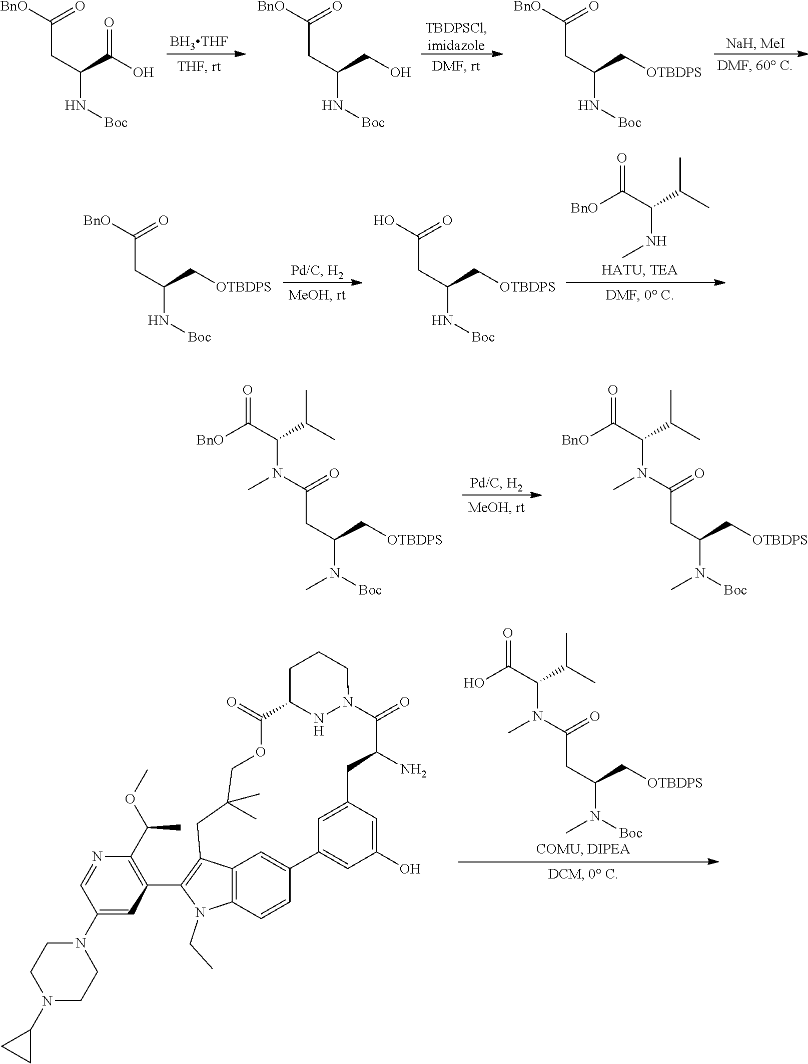

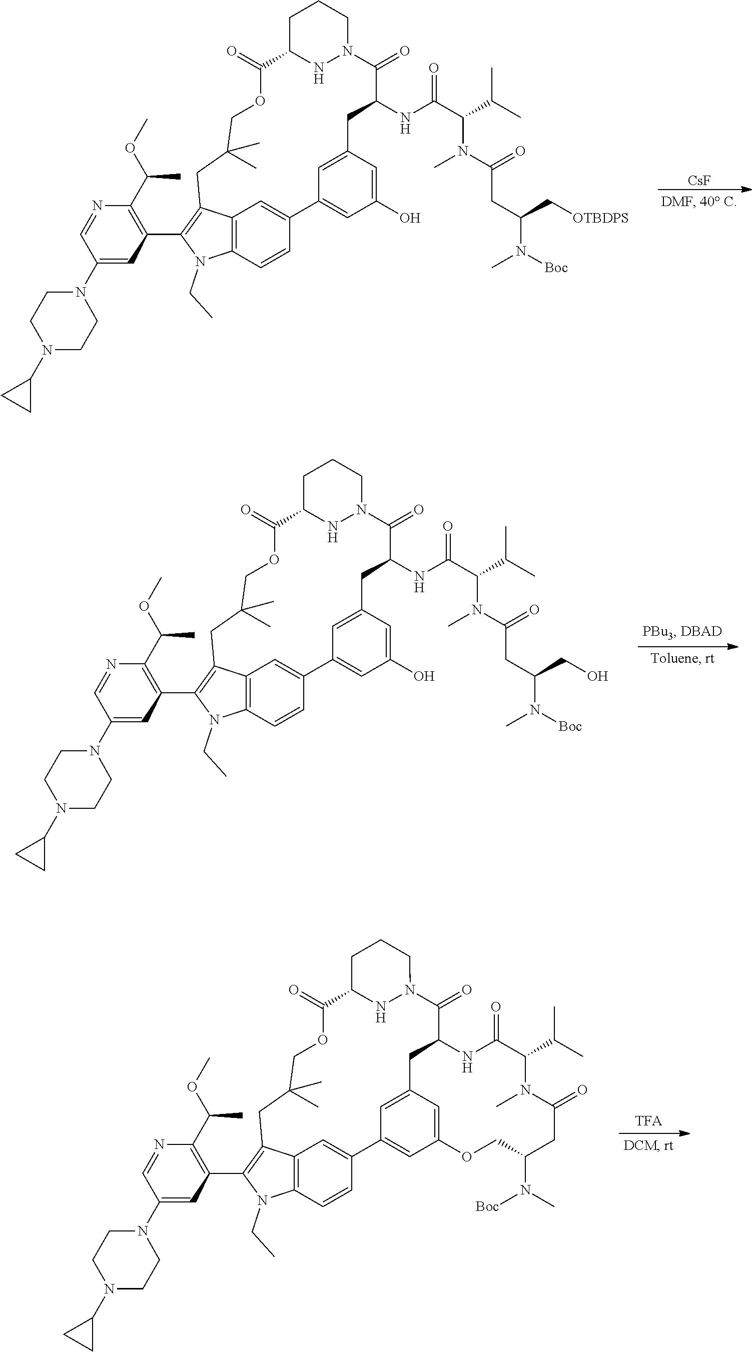

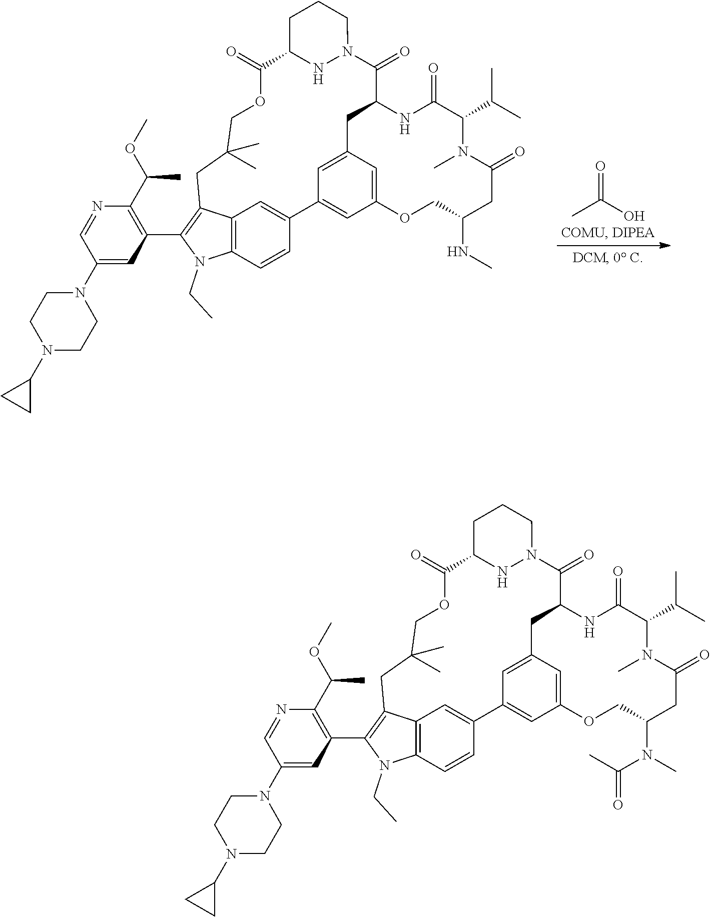

Step 1. To a solution of (S)-4-(benzyloxy)-2-((tert-butoxycarbonyl)amino)-4-oxobutanoic acid (10 g, 30.9 mmol) in THE (100 mL) stirred at 0° C. was added BH₃·THF (93 mL, 971 mmol). The resulting mixture was stirred at 3 hours at room temperature, quenched at 0° C. by the addition of MeOH (10 mL) and concentrated under reduced pressure. Purification by normal phase chromatography afforded benzyl (S)-3-((tert-butoxycarbonyl)amino)-4-hydroxybutanoate (4.69 g, 49% yield) as a yellow solid. LCMS (ESI) m/z: [M+Na+MeCN] calcd for C₁₆H₂₃NO₅: 373.2; found 373.2.

Step 2. To a solution of benzyl (S)-3-((tert-butoxycarbonyl)amino)-4-hydroxybutanoate (507 mg, 1.64 mmol) and imidazole (558 mg, 8.20 mmol) in DMF (5.0 mL) was added TBDPSCI (371 mg, 2.46 mmol) The resulting mixture was stirred for 3 h at room temperature and the reaction was then quenched by the addition of H₂O (15 mL). The resulting mixture was extracted with EtOAc (3×20 mL), and the combined organic extracts were washed with brine, dried over Na₂SO₄, filtered, and concentrated under reduced pressure to afford benzyl (S)-3-((tert-butoxycarbonyl)amino)-4-((tert-butyldiphenylsilyl)oxy)butanoate (448 mg, crude) which was used in the next reaction without further purification. LCMS (ESI) m/z: [M+H] calcd for C₃₂H₄₁NO₅Si: 548.3; found 548.5.

Step 3. To a solution of benzyl (S)-3-((tert-butoxycarbonyl)amino)-4-((tert-butyldiphenylsilyl)oxy)butanoate (1.5 g, crude) and Mel (583 mg, 4.11 mmol) in DMF (15 mL) stirred at 0° C. was added NaH (131 mg, 5.48 mmol). The resulting mixture was stirred overnight at 60° C. and the reaction was then quenched at 0° C. by the addition of cold H₂O. The aqueous mixture was extracted with EtOAc (3×50 mL), and the combined organic extracts were dried over Na₂SO₄, filtered, and concentrated under reduced pressure. The resulting residue was purified by reversed phase chromatography afforded benzyl (S)-3-((tert-butoxycarbonyl)(methyl)amino)-4-((tert-butyldiphenylsilyl)oxy)butanoate (753 mg, 25% yield over 2 steps) as a clear oil. LCMS (ESI) m/z: [M+H] calcd for C₃₃H₄₃NO₅Si: 562.3; found 562.2.

Step 4. To a solution of benzyl (S)-3-((tert-butoxycarbonyl)(methyl)amino)-4-((tert-butyldiphenylsilyl)oxy)butanoate (500 mg, 0.890 mmol) in MeOH (10 mL) was added Pd/C (167 mg). The resulting mixture was stirred for 1 h at room temperature under an atmosphere of H₂, filtered, and the filter cake washed with MeOH (3×20 mL). The filtrate was concentrated under reduced pressure to afford (S)-3-((tert-butoxycarbonyl)(methyl)amino)-4-((tert-butyldiphenylsilyl)oxy)butanoic acid (387 mg, crude) which was used in the next reaction without further purification. LCMS (ESI) m/z: [M+H] calcd for C₂₆H₃₇NO₅Si: 472.3; found 472.5.

Step 5. To a solution of (S)-3-((tert-butoxycarbonyl)(methyl)amino)-4-((tert-butyldiphenylsilyl)oxy)butanoic acid (590 mg, crude) and benzyl methyl-L-valinate (415 mg, 1.88 mmol) in DMF (12 mL) at 0° C. was added Et₃N (2.53 g, 25.0 mmol) and HATU (951 mg, 2.50 mmol). The resulting mixture was stirred for 2 h at 0° C. and the reaction was then quenched by the addition of H₂O. The aqueous mixture was extracted with EtOAc (3×30 mL), and the combined organic extracts were dried over Na₂SO₄, filtered, and concentrated under reduced pressure. Purification of the crude material by normal phase chromatography afforded benzyl N—((S)-3-((tert-butoxycarbonyl)(methyl)amino)-4-((tert-butyldiphenylsilyl)oxy)butanoyl)-N-methyl-L-valinate (776 mg, 85% yield over 2 steps) as a clear oil. LCMS (ESI) m/z: [M+H] calcd for C₃₉H₅₄N₂O₆Si: 675.4; found 675.4.

Step 6. To a solution of benzyl N—((S)-3-((tert-butoxycarbonyl)(methyl)amino)-4-((tert-butyldiphenylsilyl)oxy)butanoyl)-N-methyl-L-valinate (100 mg, 0.178 mmol) in MeOH (10 mL) was added Pd/C (100 mg). The resulting mixture was stirred for 1 h at room temperature under a hydrogen atmosphere, filtered, and the filter cake washed with MeOH (3×10 mL). The filtrate was concentrated under reduced pressure to afford N—((S)-3-((tert-butoxycarbonyl)(methyl)amino)-4-((tert-butyldiphenylsilyl)oxy)butanoyl)-N-methyl-L-valine (78 mg, crude) which was used in the next reaction without further purification. LCMS (ESI) m/z: [M+H] calcd for C₃₂H₄₈N₂O₆Si: 585.3; found 585.4.

Step 7. To a solution of (6³S,4S)-4-amino-1²-(5-(4-cyclopropylpiperazin-1-yl)-2-((S)-1-methoxyethyl)pyridin-3-yl)-1¹-ethyl-2⁵-hydroxy-10,10-dimethyl-6¹,6²,6³,6⁴,6⁵,6⁶-hexahydro-1 ¹H-8-oxa-1(5,3)-indola-6(1,3)-pyridazina-2(1,3)-benzenacycloundecaphane-5,7-dione (613 mg, 0.802 mmol) and N—((S)-3-((tert-butoxycarbonyl)(methyl)amino)-4-((tert-butyldiphenylsilyl)oxy)butanoyl)-N-methyl-L-valine (610 mg, crude) in DCM (10 mL) at 0° C. was added DIPEA (2.07 g, 16.0 mmol) and COMU (412 mg, 0.962 mmol). The resulting mixture was stirred for 2 h at 0° C. and the reaction was then quenched by the addition of H₂O. The aqueous layer was extracted with DCM (3×30 mL) and the combined organic extracts were dried over Na₂SO₄, filtered, and concentrated under reduced pressure. Purification by preparative normal phase prep-TLC afforded tert-butyl ((2S)-1-((tert-butyldiphenylsilyl)oxy)-4-(((2S)-1-(((6³S,4S)-1²-(5-(4-cyclopropylpiperazin-1-yl)-2-((S)-1-methoxyethyl)pyridin-3-yl)-1¹-ethyl-2⁵-hydroxy-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)- benzenacycloundecaphane-4-yl)amino)-3-methyl-1-oxobutan-2-yl)(methyl)amino)-4-oxobutan-2-yl)(methyl)carbamate (352 mg, 33% yield) as a yellow oil. LCMS (ESI) m/z: [M+H] calcd for C₇₆H₁₀₃N₉O₁₀Si: 1330.8; found 1331.1.

Step 8. To a solution of tert-butyl ((2S)-1-((tert-butyldiphenylsilyl)oxy)-4-(((2S)-1-(((6³S,4S)-1²-(5-(4-cyclopropylpiperazin-1-yl)-2-((S)-1-methoxyethyl)pyridin-3-yl)-1¹-ethyl-2⁵-hydroxy-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)amino)-3-methyl-1-oxobutan-2-yl)(methyl)amino)-4-oxobutan-2-yl)(methyl)carbamate (340 mg, 0.255 mmol) in DMF (3.0 mL) stirred at room temperature was added CsF (116 mg, 0.765 mmol). The resulting mixture was stirred overnight at 40° C. and the reaction was then quenched at 0° C. by the addition of cold H₂O. The aqueous mixture was extracted with EtOAc (3×20 mL), and the combined organic extracts were dried over Na₂SO₄, filtered, and concentrated under reduced pressure to afford tert-butyl ((2S)-4-(((2S)-1-(((6³S,4S)-12-(5-(4-cyclopropylpiperazin-1-yl)-2-((S)-1-methoxyethyl)pyridin-3-yl)-1¹-ethyl-2⁵-hydroxy-10,10-dimethyl-5,7-dioxo-6¹,6²,6³,6⁴,6⁵,6⁶-hexahydro-11H-8-oxa-1(5,3)-indola-6(1,3)-pyridazina-2(1,3)-benzenacycloundecaphane-4-yl)amino)-3-methyl-1-oxobutan-2-yl)(methyl)amino)-1-hydroxy-4-oxobutan-2-yl)(methyl)carbamate (300 mg, crude), which was used in the next reaction without further purification. LCMS (ESI) m/z: [M+H] calcd for C₆₀H₈₅N₉O₁₀: 1092.7; found 1092.9.

Step 9. To a solution of tert-butyl ((2S)-4-(((2S)-1-(((6³S,4S)-1²-(5-(4-cyclopropylpiperazin-1-yl)-2-((S)-1-methoxyethyl)pyridin-3-yl)-1¹-ethyl-2⁵-hydroxy-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)amino)-3-methyl-1-oxobutan-2-yl)(methyl)amino)-1-hydroxy-4-oxobutan-2-yl)(methyl)carbamate (400 mg, crude) in toluene (20 mL) was added DBAD (422 mg, 1.83 mmol) and Bu₃P (370 mg, 1.83 mmol) The resulting mixture was stirred for 2 h at room temperature and the reaction was quenched at room temperature by the addition of H₂O. The aqueous layer was extracted with EtOAc (3×30 mL), dried over Na₂SO₄, filtered, and concentrated under reduced pressure. Purification of the crude residue by preparative normal phase prep-TLC afforded tert-butyl ((9S,15S,18S,22S)-3-(5-(4-cyclopropylpiperazin-1-yl)-2-((S)-1-methoxyethyl)pyridin-3-yl)-2-ethyl-18-isopropyl-5,5,19-trimethyl-8,14,17,20-tetraoxo-2,4,5,6,9,10,11,12,14,15,16,17,18,19,20,21,22,23-octadecahydro-8H-9,13-epimino-1,30-etheno-15,27-methano-25,29-(metheno)pyrrolo[3,4-v][1,18]dioxa[6,9,12]triazacyclotriacontin-22-yl)(methyl)carbamate (263 mg, contains Bu₃PO impurity) as a clear oil. LCMS (ESI) m/z: [M+H] calcd for C₆₀H₈₃N₉O₉: 1074.6; found 1074.7.

Step 10. To a solution of tert-butyl ((9S,15S,18S,22S)-3-(5-(4-cyclopropylpiperazin-1-yl)-2-((S)-1-methoxyethyl)pyridin-3-yl)-2-ethyl-18-isopropyl-5,5,19-trimethyl-8,14,17,20-tetraoxo-2,4,5,6,9,10,11,12,14,15,16,17,18,19,20,21,22,23-octadecahydro-8H-9,13-epimino-1,30-etheno-15,27-methano-25,29-(metheno)pyrrolo[3,4-v][1,18]dioxa[6,9,12]triazacyclotriacontin-22-yl)(methyl)carbamate (220 mg, 0.205 mmol) in DCM (10 mL) stirred at 0° C. was added TFA (5.0 mL). The resulting mixture was stirred for 1 h at room temperature and then neutralized to pH=7 at 0° C. by the addition of sat. aq. NaHCO₃. The aqueous layer was extracted with DCM (3×10 mL) and the combined organic extracts were washed with brine, dried over Na₂SO₄, filtered, and concentrated under reduced pressure to afford (9S,15S,18S,22S)-3-(5-(4-cyclopropylpiperazin-1-yl)-2-((S)-1-methoxyethyl)pyridin-3-yl)-2-ethyl-18-isopropyl-5,5,19-trimethyl-22-(methylamino)-2,4,5,6,9,10,11,12,15,16,18,19,22,23-tetradecahydro-8H-9,13-epimino-1,30-etheno-15,27-methano-25,29-(metheno)pyrrolo[3,4-v][1,18]dioxa[6,9,12]triazacyclotriacontine-8,14,17,20(21H)-tetraone (180 mg, crude) which was used in the next reaction without further purification. LCMS (ESI) m/z: [M+H] calcd for C₅₅H₇₅N₉O₇: 974.6; found 974.7.

Step 11. To a solution of (9S,15S,18S,22S)-3-(5-(4-cyclopropylpiperazin-1-yl)-2-((S)-1-methoxyethyl)pyridin-3-yl)-2-ethyl-18-isopropyl-5,5,19-trimethyl-22-(methylamino)-2,4,5,6,9,10,11,12,15,16,18,19,22,23-tetradecahydro-8H-9,13-epimino-1,30-etheno-15,27-methano-25,29-(metheno)pyrrolo[3,4-v][1,18]dioxa[6,9,12]triazacyclotriacontine-8,14,17,20(21H)-tetraone (47 mg, crude) and DIPEA (125 mg, 0.960 mmol) in DCM (2.0 mL) stirred at 0° C. was added acetic acid (5.8 mg, 0.096 mmol) and COMU (30.9 mg, 0.072 mmol). The resulting mixture was stirred for 1 h at 0° C. and the reaction was then quenched by the addition of H₂O. The aqueous layer was extracted with DCM (3×20 mL) and the combined organic extracts were dried over Na₂SO₄, filtered, and concentrated under reduced pressure. Purification by preparative normal phase prep-TLC followed by reversed phase chromatography afforded N-((9S,15S,18S,22S)-3-(5-(4-cyclopropylpiperazin-1-yl)-2-((S)-1-methoxyethyl)pyridin-3-yl)-2-ethyl-18-isopropyl-5,5,19-trimethyl-8,14,17,20-tetraoxo-2,4,5,6,9,10,11,12,14,15,16,17,18,19,20,21,22,23-octadecahydro-8H-9,13-epimino-1,30-etheno-15,27-methano-25,29-(metheno)pyrrolo[3,4-v][1,18]dioxa[6,9,12]triazacyclotriacontin-22-yl)-N-methylacetamide (4.2 mg, 7.7% yield over 2 steps) as a white solid. LCMS (ESI) m/z: [M+H] calcd for C₅₇H₇₇N₉O₈: 1016.6; found 1016.9. ¹H NMR (400 MHz, DMSO-d₆) δ 8.54-8.36 (m, 2H), 7.98 (s, 1H), 7.68 (d, J=8.4 Hz, 2H), 7.57 (d, J=8.8 Hz, 1H), 7.32 (s, 1H), 7.20 (d, J=5.3 Hz, 2H), 6.78-6.59 (m, 1H), 5.32-5.15 (m, 2H), 4.84-4.71 (m, 1H), 4.61-4.51 (m, 1H), 4.30-4.09 (m, 4H), 3.99-3.88 (m, 1H) 3.68-3.60 (m, 1H), 3.28-3.09 (m, 3H), 2.98-2.64 (m, 14H), 2.38-2.27 (m, 1H), 2.12-1.99 (m, 4H), 1.90-1.65 (d, 4H), 1.58-1.47 (m, 1H), 1.43-1.16 (m, 7H), 0.98-0.70 (m, 15H), 0.53-0.20 (m, 8H).

### Example A149. Synthesis of (3aS,9S,15S,32aS)-21-(5-(4-cyclopropylpiperazin-1-yl)-2-((S)-1-methoxyethyl)pyridin-3-yl)-22-ethyl-6-isopropyl-19,19-dimethyl-1-propionyl-2,3,3a,4,8,9,14,15,18,19,20,22,32,32a-tetradecahydro-1H,6H,12H-11,15-epimino-23,25-etheno-9,28-methano-26,30-(metheno)dipyrrolo[2,3-b1:3′,4′-q][1,13,26]trioxa[4,7]diazacyclotriacontine-7,10,16(13H)-trione

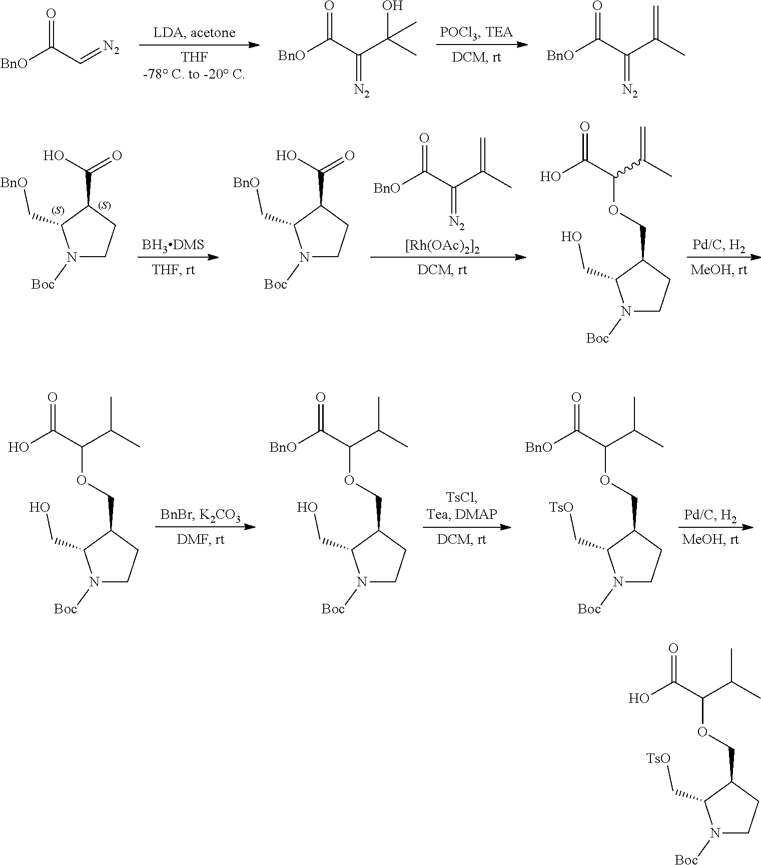

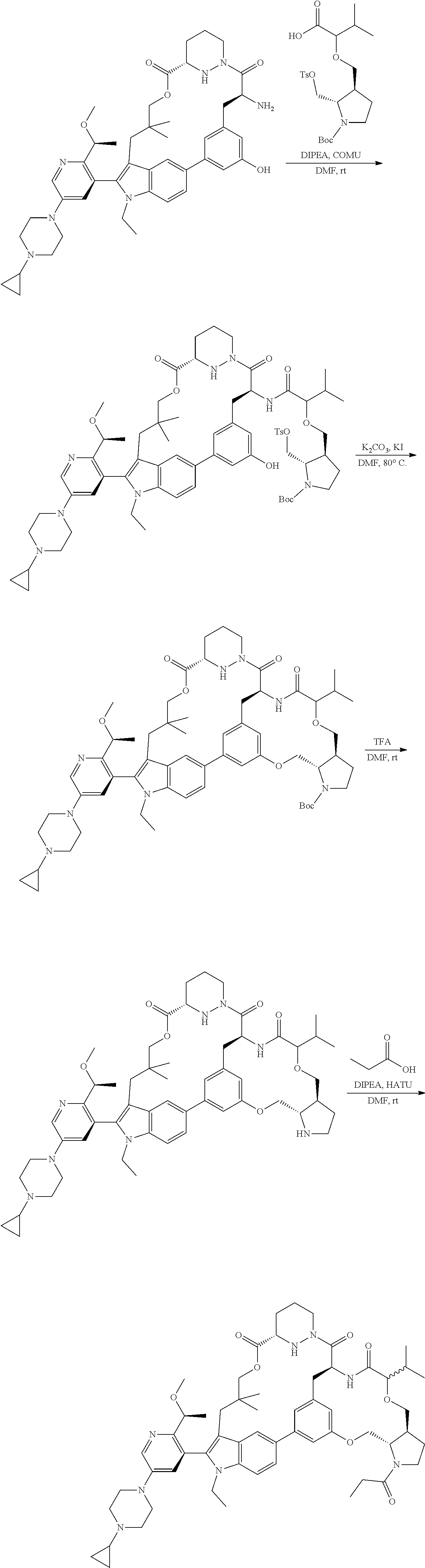

Step 1. To a stirred solution of benzyl 2-diazoacetate (3.00 g, 17.0 mmol) in THE (60 mL) at −78° C. was added LDA (3.65 g, 34.1 mmol) by acetone (4.95 g, 85.1 mmol). The resulting mixture was warmed over 1 h from −78° C. to −20° C. and was then quenched at −50° C. by the addition of sat. aq. NH₄Cl. The aqueous mixture was extracted with EtOAc (3×20 mL), the combined organic extracts concentrated under reduced pressure, and the crude residue purified by normal phase prep-TLC to afford benzyl 2-diazo-3-hydroxy-3-methylbutanoate (1.01 g, 25% yield) as a yellow oil. LCMS (ESI) m/z: [M−N₂+H+Na] calcd for C₁₂H₁₄N₂O₃: 230.1; found 230.2.

Step 2. To a solution of benzyl 2-diazo-3-hydroxy-3-methylbutanoate (1.01 g, 4.31 mmol) and Et₃N (6.98 g, 68.9 mmol) in DCM (15 mL) at 0° C. was added POCl₃ (1.98 g, 12.9 mmol). The resulting mixture was stirred for 2 h at room temperature and the reaction was then quenched at 0° C. by the addition of H₂O (20 mL). The aqueous phase was extracted with DCM (3×15 mL) and the combined organic extracts were concentrated under reduced pressure. Purification by normal phase prep-TLC afforded benzyl 2-diazo-3-methylbut-3-enoate (639 mg, 69% yield) as a yellow oil. ¹H NMR (400 MHz, CDCl₃) δ 7.29-7.42 (m, 5H), 5.36 (br s, 1H), 4.82 (br s, 1H), 1.95 (s, 3H).

Step 3. To a solution of (2S,3S)-2-((benzyloxy)methyl)-1-(tert-butoxycarbonyl)pyrrolidine-3-carboxylic acid (4.0 g, 11.9 mmol) in THE (40 mL) at 0° C. was added BH₃·DMS (1.79 mL, 18.9 mmol). The resulting mixture was stirred for 1 h at room temperature, quenched at 0° C. by the addition of MeOH (50 mL), and concentrated under reduced pressure. Purification by reversed phase chromatography afforded tert-butyl (2S,3S)-2-((benzyloxy)methyl)-3-(hydroxymethyl)pyrrolidine-1-carboxylate (3.13 g, 69% yield) as a clear oil. LCMS (ESI) m/z: [M+H] calcd for C₁₈H₂₇NO₄: 322.2; found 321.9.

Step 4. To a solution of benzyl 2-diazo-3-methylbut-3-enoate (888 mg, 4.12 mmol) and tert-butyl (2S,3S)-2-((benzyloxy)methyl)-3-(hydroxymethyl)pyrrolidine-1-carboxylate (440 mg, 1.37 mmol) in DCM (10 mL) at room temperature was added rhodium(II) acetate dimer (30 mg, 0.068 mmol). The resulting mixture was stirred for 12 h at room temperature and the reaction was then quenched by the addition of H₂O (20 mL). The aqueous phase was extracted with DCM (3×20 mL) and the combined organic extracts were concentrated under reduced pressure. Purification of the crude material by reversed phase chromatography afforded tert-butyl (2S,3S)-3-(((1-(benzyloxy)-3-methyl-1-oxobut-3-en-2-yl)oxy)methyl)-2-((benzyloxy)methyl)pyrrolidine-1-carboxylate (311 mg, 45% yield, brown oil) as a mixture of diastereomers. LCMS (ESI) m/z: [M+H] calcd for C₃₀H₃₉NO₆: 510.3; found 510.2.

Step 5. To a solution of tert-butyl (2S,3S)-3-(((1-(benzyloxy)-3-methyl-1-oxobut-3-en-2-yl)oxy)methyl)-2-((benzyloxy)methyl)pyrrolidine-1-carboxylate (311 mg, 0.610 mmol) in MeOH (4 mL) was added 10% Pd/C (150 mg) under an atmosphere of N₂. The resulting mixture was stirred for 10 h at room temperature under an atmosphere of H₂, filtered, and the filter cake washed with MeOH (10×10 mL). The filtrate was concentrated under reduced pressure to afford 2-(((2S,3S)-1-(tert-butoxycarbonyl)-2-(hydroxymethyl)pyrrolidin-3-yl)methoxy)-3-methylbutanoic acid (206 mg, crude), which was used without further purification. LCMS (ESI) m/z: [M+H] calcd for C₁₆H₂₉NO₆: 332.2; found 332.1.

Step 6. To a solution of 2-(((2S,3S)-1-(tert-butoxycarbonyl)-2-(hydroxymethyl)pyrrolidin-3-yl)methoxy)-3-methylbutanoic acid (206 mg, crude) and K₂CO₃ (172 mg, 1.24 mmol) in DMF (3.0 mL) at room temperature was added benzyl bromide (117 mg, 0.684 mmol). The resulting mixture was stirred for 3 h and the reaction was then quenched by the addition of H₂O (15 mL). The aqueous mixture was extracted with EtOAc (3×15 mL), and the combined organic extracts were concentrated under reduced pressure. Purification of the residue by reversed phase chromatography afforded tert-butyl (2S,3S)-3-(((1-(benzyloxy)-3-methyl-1-oxobutan-2-yl)oxy)methyl)-2-(hydroxymethyl)pyrrolidine-1-carboxylate (214 mg, 83% yield over 2 steps) as a clear oil. LCMS (ESI) m/z: [M+H] calcd for C₂₃H₃₅NO₆: 422.3; found 422.2.

Step 7. To a solution of tert-butyl (2S,3S)-3-(((1-(benzyloxy)-3-methyl-1-oxobutan-2-yl)oxy)methyl)-2-(hydroxymethyl)pyrrolidine-1-carboxylate (214 mg, 0.508 mmol) and Et₃N (462 mg, 4.57 mmol) in DCM (5.0 mL) at 0° C. were added DMAP (31.0 mg, 0.254 mmol) and tosyl chloride (435 mg, 2.29 mmol). The resulting mixture was stirred for 10 h at room temperature and the reaction was then quenched at 0° C. by the addition of H₂O (15 mL). The aqueous phase was extracted with DCM (3×15 mL) and the combined organic extracts were concentrated under reduced pressure. Purification of the crude residue by reversed phase chromatography afforded tert-butyl (2S,3S)-3-(((1-(benzyloxy)-3-methyl-1-oxobutan-2-yl)oxy)methyl)-2-((tosyloxy)methyl)pyrrolidine-1-carboxylate (234 mg, 80% yield) as a clear oil. LCMS (ESI) m/z: [M−C₅H₈O₂+H] calcd for C₃₀H₄₁NO₈S: 476.2; found 476.3.

Step 8. To a solution of tert-butyl (2S,3S)-3-(((1-(benzyloxy)-3-methyl-1-oxobutan-2-yl)oxy)methyl)-2-((tosyloxy)methyl)pyrrolidine-1-carboxylate (234 mg, 0.406 mmol) in MeOH (3.0 mL) was added 10% Pd/C (151 mg) under an atmosphere of N₂. The resulting mixture was stirred for 1 h at room temperature under an atmosphere of H₂, filtered, and the filter cake washed with MeOH (10×10 mL). The filtrate was concentrated under reduced pressure to afford 2-(((2S,3S)-1-(tert-butoxycarbonyl)-2-((tosyloxy)methyl)pyrrolidin-3-yl)methoxy)-3-methylbutanoic acid (199 mg, crude) which was used without further purification. LCMS (ESI) m/z: [M−C₅H₈O₂+H] calcd for C₂₃H₃₅NO₈S: 386.2; found 386.0.

Step 9. To a solution of (6³S,4S)-4-amino-1²-(5-(4-cyclopropylpiperazin-1-yl)-2-((S)-1-methoxyethyl)pyridin-3-yl)-1¹-ethyl-2⁵-hydroxy-10,10-dimethyl-6¹,6²,6³,6⁴,6⁵,6⁶-hexahydro-1 ¹H-8-oxa-1(5,3)-indola-6(1,3)-pyridazina-2(1,3)-benzenacycloundecaphane-5,7-dione (230 mg, 0.301 mmol) and DIPEA (1.56 g, 12.0 mmol) in DMF (3.0 mL) at 0° C. were added 2-(((2S,3S)-1-(tert-butoxycarbonyl)-2-((tosyloxy)methyl)pyrrolidin-3-yl)methoxy)-3-methylbutanoic acid (132 mg, crude) and COMU (193 mg, 0.452 mmol). The resulting mixture was stirred for 1 h and the reaction was then quenched by the addition of H₂O (10 mL). The aqueous mixture was extracted with EtOAc (3×5 mL), and the combined organic extracts were treated with brine, dried over Na₂SO₄, filtered, and concentrated under reduced pressure. Purification by normal phase prep-TLC afforded tert-butyl (2S,3S)-3-(((1-(((6³S,4S)-1²-(5-(4-cyclopropylpiperazin-1-yl)-2-((S)-1-methoxyethyl)pyridin-3-yl)-1¹-ethyl-2⁵-hydroxy-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)amino)-3-methyl-1-oxobutan-2-yl)oxy)methyl)-2-((tosyloxy)methyl)pyrrolidine-1-carboxylate (157 mg, 47% yield over 2 steps) as a clear oil. LCMS (ESI) m/z: [M+H] calcd for C₆₇H₉₀N₈O₁₂S: 1231.7; found 1231.9.

Step 10. To a solution of tert-butyl (2S,3S)-3-(((1-(((6³S,4S)-1²-(5-(4-cyclopropylpiperazin-1-yl)-2-((S)-1-methoxyethyl)pyridin-3-yl)-1¹-ethyl-2⁵-hydroxy-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)amino)-3-methyl-1-oxobutan-2-yl)oxy)methyl)-2-((tosyloxy)methyl)pyrrolidine-1-carboxylate (157 mg, 0.127 mmol) and KI (21.2 mg, 0.127 mmol) in DMF (16 mL) at room temperature was added K₂CO₃ (176 mg, 1.27 mmol). The resulting mixture was stirred for 2.5 h at 80° C. and the reaction was then quenched at −30° C. by the addition of H₂O (30 mL). The aqueous mixture was extracted with EtOAc (3×10 mL) and combined organic extracts were washed with brine, dried over Na₂SO₄, filtered, and concentrated under reduced pressure. Purification of the crude residue by reversed phase chromatography afforded tert-butyl (3aS,9S,15S,32aS)-21-(5-(4-cyclopropylpiperazin-1-yl)-2-((S)-1-methoxyethyl)pyridin-3-yl)-22-ethyl-6-isopropyl-19,19-dimethyl-7,10,16-trioxo-2,3,3a,4,7,8,9,10,13,14,15,16,18,19,20,22,32,32a-octadecahydro-1H,6H,12H-11,15-epimino-23,25-etheno-9,28-methano-26,30-(metheno)dipyrrolo[2,3-b1:3′,4′-q][1,13,26]trioxa[4,7]diazacyclotriacontine-1-carboxylate (54 mg, 41% yield) as a white solid. LCMS (ESI) m/z: [M+H] calcd for C₆₀H₈₂N₈O₉: 1059.6; found 1059.5.

Step 11. To a solution of tert-butyl (3aS,9S,15S,32aS)-21-(5-(4-cyclopropylpiperazin-1-yl)-2-((S)-1-methoxyethyl)pyridin-3-yl)-22-ethyl-6-isopropyl-19,19-dimethyl-7,10,16-trioxo-2,3,3a,4,7,8,9,10,13,14,15,16,18,19,20,22,32,32a-octadecahydro-1H,6H,12H-11,15-epimino-23,25-etheno-9,28-methano-26,30-(metheno)dipyrrolo[2,3-b1:3′,4′-q][1,13,26]trioxa[4,7]diazacyclotriacontine-1-carboxylate (54 mg, 0.051 mmol) in DCM (2.0 mL) at 0° C. was added TFA (400 mL). The resulting mixture was stirred for 2 h at room temperature, diluted with DCM (10 mL), and concentrated under reduced pressure to afford (3aS,9S,15S,32aS)-21-(5-(4-cyclopropylpiperazin-1-yl)-2-((S)-1-methoxyethyl)pyridin-3-yl)-22-ethyl-6-isopropyl-19,19-dimethyl-2,3,3a,4,8,9,14,15,18,19,20,22,32,32a-tetradecahydro-1H,6H,12H-11,15-epimino-23,25-etheno-9,28-methano-26,30-(metheno)dipyrrolo[2,3-b1:3′,4′-q][1,13,26]trioxa[4,7]diazacyclotriacontine-7,10,16(13H)-trione (103 mg, crude) which was used without further purification. LCMS (ESI) m/z: [M+H] calcd for C₅₅H₇₄N₈O₇: 959.6; found 959.7.

Step 12. To a solution of (3aS,9S,15S,32aS)-21-(5-(4-cyclopropylpiperazin-1-yl)-2-((S)-1-methoxyethyl)pyridin-3-yl)-22-ethyl-6-isopropyl-19,19-dimethyl-2,3,3a,4,8,9,14,15,18,19,20,22,32,32a-tetradecahydro-1H,6H,12H-11,15-epimino-23,25-etheno-9,28-methano-26,30-(metheno)dipyrrolo[2,3-b1:3′,4′-q][1,13,26]trioxa[4,7]diazacyclotriacontine-7,10,16(13H)-trione (95 mg, 0.099 mmol) and DIPEA (256 mg, 1.98 mmol) in DMF (3 mL) at 0° C. were added propanoic acid (14.7 mg, 0.198 mmol) and HATU (56.5 mg, 0.149 mmol). The resulting mixture was stirred for 1 h and the reaction was then quenched at 0° C. by the addition of H₂O. The aqueous mixture was extracted with EtOAc (3×10 mL), and the combined organic extracts were treated with brine, dried over Na₂SO₄, filtered, and concentrated under reduced pressure. Purification of the crude residue by reversed phase chromatography afforded (3aS,9S,15S,32aS)-21-(5-(4-cyclopropylpiperazin-1-yl)-2-((S)-1-methoxyethyl)pyridin-3-yl)-22-ethyl-6-isopropyl-19,19-dimethyl-1-propionyl-2,3,3a,4,8,9,14,15,18,19,20,22,32,32a-tetradecahydro-1H,6H,12H-11,15-epimino-23,25-etheno-9,28-methano-26,30-(metheno)dipyrrolo[2,3-b1:3′,4′-q][1,13,26]trioxa[4,7]diazacyclotriacontine-7,10,16(13H)-trione (19.6 mg, 19% yield, white solid) as a mixture of diastereomers. LCMS (ESI) m/z: [M+H] calcd for C₅₈H₇₈N₈O₈: 1015.6; found 1015.8. ¹H NMR (400 MHz, DMSO-d₆) δ 8.63-7.97 (m, 3H), 7.84-7.66 (m, 1H), 7.62 (t, J=8.0 Hz, 1H), 7.53-7.46 (m, 1H), 7.37-6.96 (m, 3H), 5.65-4.73 (m, 3H), 4.62-4.41 (m, 1H), 4.38-3.94 (m, 6H), 3.81-3.68 (m, 2H), 3.64 (d, J=2.3 Hz, 2H), 3.35-3.21 (m, 4H), 3.20-2.92 (m, 3H), 2.89-2.66 (m, 5H), 2.63-2.47 (m, 5H), 2.46-1.84 (m, 6H), 1.81-1.65 (m, 7H), 1.59-1.54 (m, 3H), 1.49-1.08 (m, 7H), 1.07-0.82 (m, 9H), 0.69-0.31 (m, 6H).

### Example A192. Synthesis of isobutyl (3aS,6S,9S,15S,32aS)-21-(5-(4-cyclopropylpiperazin-1-yl)-2-((S)-1-methoxyethyl)pyridin-3-yl)-22-ethyl-6-isopropyl-5,19,19-trimethyl-4,7,10,16-tetraoxo-2,3,3a,4,5,6,7,8,9,10,13,14,15,16,18,19,20,22,32,32a-icosahydro-1H,12H-11,15-epimino-23,25-etheno-9,28-methano-26,30-(metheno)dipyrrolo[2,3-c:3′,4′-v][1,18]dioxa[6,9,12]triazacyclotriacontine-1-carboxylate

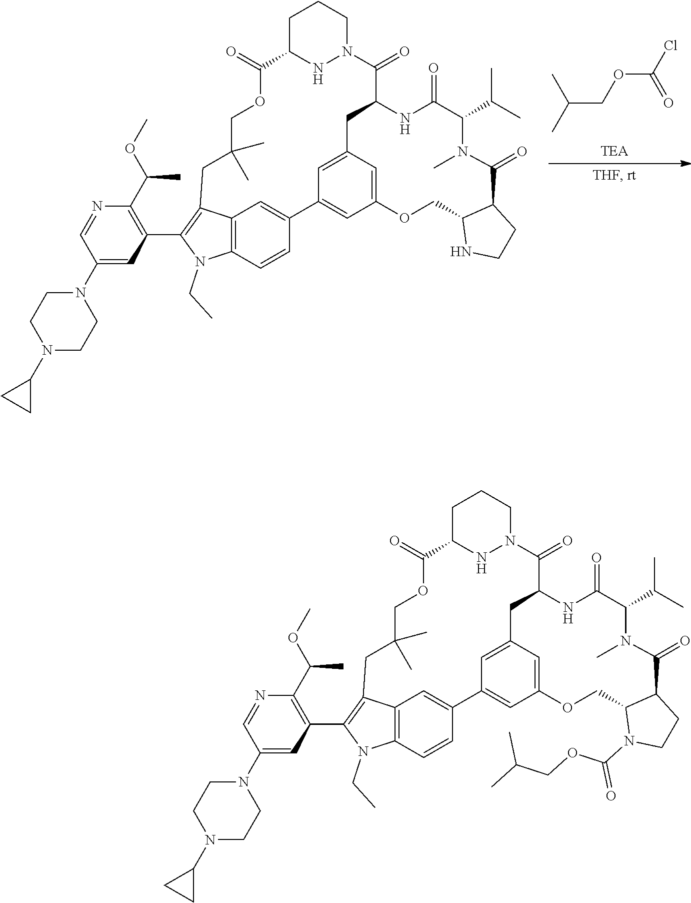

Step 1. To a solution of (3aS,6S,9S,15S,32aS)-21-(5-(4-cyclopropylpiperazin-1-yl)-2-((S)-1-methoxyethyl)pyridin-3-yl)-22-ethyl-6-isopropyl-5,19,19-trimethyl-3,3a,5,6,8,9,14,15,18,19,20,22,32,32a-tetradecahydro-1H,12H-11,15-epimino-23,25-etheno-9,28-methano-26,30-(metheno)dipyrrolo[2,3-c:3′,4′-v][1,18]dioxa[6,9,12]triazacyclotriacontine-4,7,10,16(2H,13H)-tetraone (120 mg, 0.122 mmol) and Et₃N (123 mg, 1.22 mmol) in THE (3.0 mL) at 0° C. was added isobutyl chloroformate (33.2 mg, 0.244 mmol). The resulting mixture was stirred for 2 h at room temperature and the reaction was then quenched at 0° C. by the addition of H₂O (5 mL). The aqueous mixture was extracted with EtOAc (2×5 mL), and the combined organic extracts were dried over Na₂SO₄, filtered, and concentrated under reduced pressure. Purification by reversed phase chromatography afforded isobutyl (3aS,6S,9S,15S,32aS)-21-(5-(4-cyclopropylpiperazin-1-yl)-2-((S)-1-methoxyethyl)pyridin-3-yl)-22-ethyl-6-isopropyl-5,19,19-trimethyl-4,7,10,16-tetraoxo-2,3,3a,4,5,6,7,8,9,10,13,14,15,16,18,19,20,22,32,32a-icosahydro-1H,12H-11,15-epimino-23,25-etheno-9,28-methano-26,30-(metheno)dipyrrolo[2,3-c:3′,4′-v][1,18]dioxa[6,9,12]triazacyclotriacontine-1-carboxylate (47.6 mg 35% yield) as a white solid. LCMS (ESI) m/z: [M+H] calcd for C₆₁H₈₃N₉O₉: 1086.6; found 1086.4. ¹H NMR (400 MHz, DMSO-d₆) δ 8.45 (s, 1H), 8.24-7.97 (m, 1H), 7.71 (br s, 1H), 7.56 (d, J=8.8 Hz, 1H), 7.50-7.32 (m, 2H), 7.25-7.13 (m, 2H), 6.89-6.62 (m, 1H), 5.42-5.25 (m, 1H), 5.21-4.94 (m, 1H), 4.72-4.65 (m, 1H), 4.60-4.34 (m, 2H), 4.31-4.16 (m, 2H), 4.14-4.04 (m, 2H), 3.97-3.78 (m, 3H), 3.74-3.62 (m, 2H), 3.53-3.48 (m, 1H), 3.23-3.12 (m, 6H), 3.04 (s, 2H), 3.01-2.94 (m, 1H), 2.92-2.79 (m, 2H), 2.74-2.61 (m, 6H), 2.42-2.36 (m, 1H), 2.30-2.12 (m, 2H), 2.10-1.98 (m, 2H), 1.94-1.80 (m, 2H), 1.78-1.53 (m, 5H), 1.38-1.17 (m, 6H), 1.00 (t, J=7.2 Hz, 2H), 0.94-0.72 (m, 12H), 0.68 (d, J=6.9 Hz, 3H), 0.58 (s, 2H), 0.52-0.42 (m, 3H), 0.34 (s, 2H).

### Example A194. Synthesis of (3aS,6S,9S,15S,32aR)-21-(5-(4-cyclopropylpiperazin-1-yl)-2-((S)-1-methoxyethyl)pyridin-3-yl)-22-ethyl-6-isopropyl-5,19,19-trimethyl-1-propionyl-2,3,3a,5,6,8,9,14,15,18,19,20,22,31,32,32a-hexadecahydro-12H-11,15-epimino-23,25-etheno-9,28-methano-26,30-(metheno)dipyrrolo[3,2-o:3′,4′-z][1]oxa[7,10,13]triazacyclotriacontine-4,7,10,16(1H,13H)-tetraone

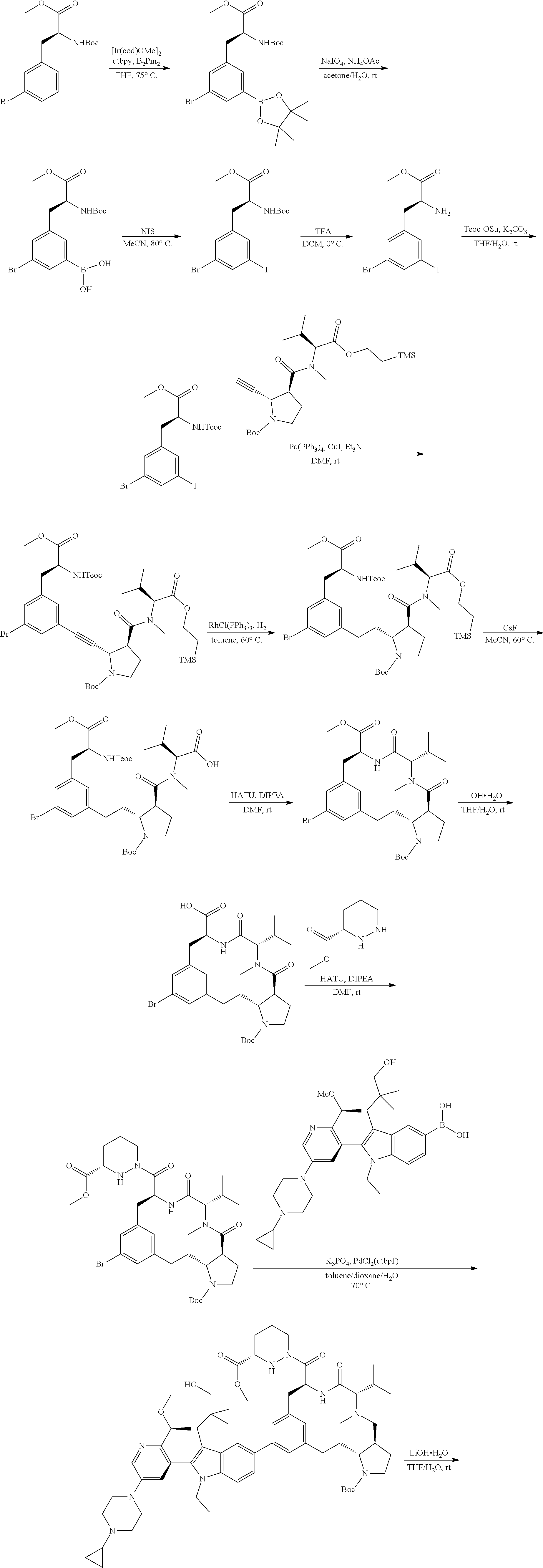

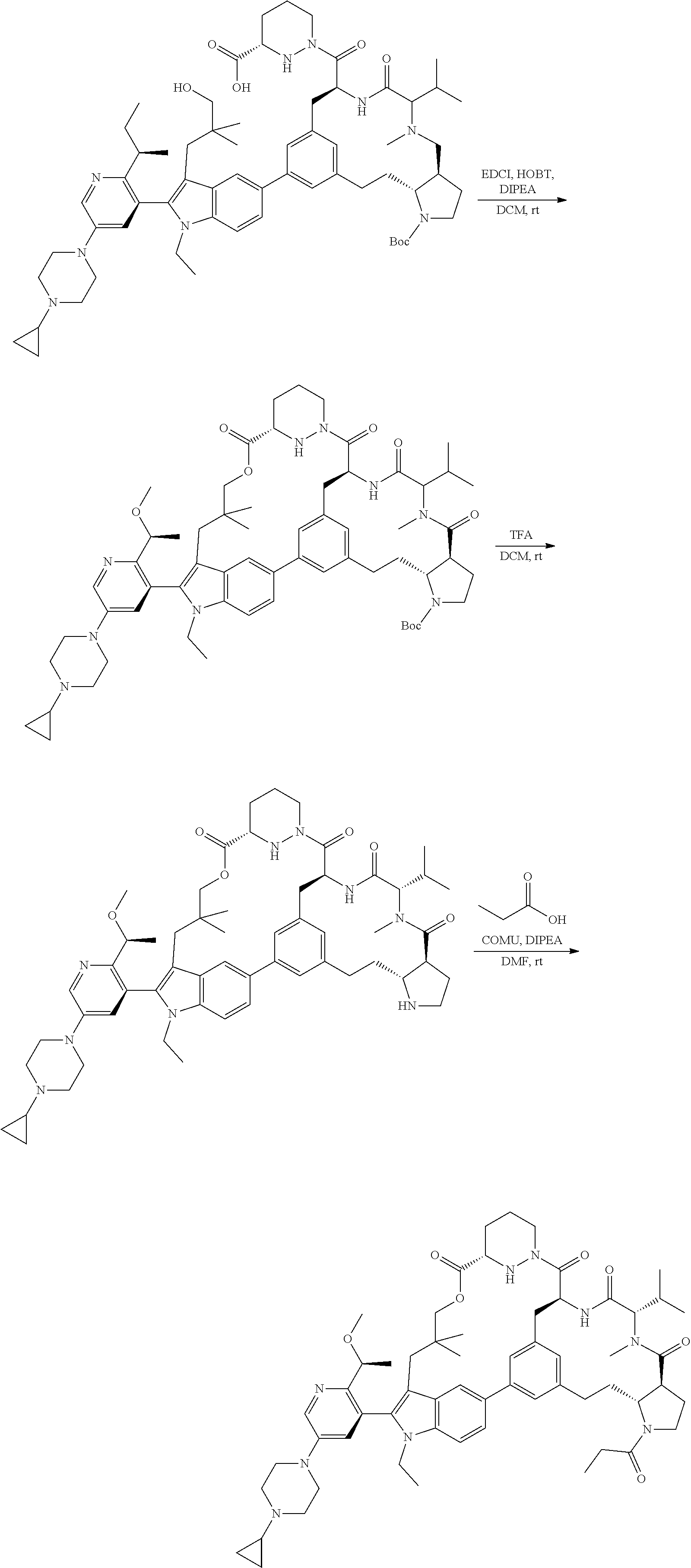

Step 1. To a solution of methyl (S)-3-(3-bromophenyl)-2-((tert-butoxycarbonyl)amino)propanoate (50 g, 139 mmol) and 4,4′-di-tert-butyl-2,2′-dipyridyl (5.62 g, 20.9 mmol) in THF (200 mL) at room temperature were added [Ir(cod)OMe]2 (2.78 g, 4.19 mmol) and 4,4,4′,4′,5,5,5′,5′-octamethyl-2,2′-bi(1,3,2-dioxaborolane) (36.7 g, 140 mmol). The resulting mixture was stirred overnight at 75° C. and was then quenched at 0° C. by the addition of H₂O. The aqueous mixture was extracted with EtOAc (3×500 mL), and the combined organic extracts were dried over Na₂SO₄, filtered, and concentrated under reduced pressure to afford methyl (S)-3-(3-bromo-5-(4,4,5,5-tetramethyl-1,3,2-dioxaborolan-2-yl)phenyl)-2-((tert-butoxycarbonyl)amino)propanoate (70.0 g, crude) which was taken to the next reaction without further purification. LCMS (ESI) m/z: [M+H−56] calcd for C₂₁H₃₁BBrNO₆: 428.1; found 428.1.

Step 2. To a solution of methyl (S)-3-(3-bromo-5-(4,4,5,5-tetramethyl-1,3,2-dioxaborolan-2-yl)phenyl)-2-((tert-butoxycarbonyl)amino)propanoate (70.0 g, crude) and NaIO₄ (155 g, 722 mmol) in acetone (500 mL) at room temperature was added a solution of NH₄OAc (55.7 g, 722 mmol) in H₂O (250 mL). The resulting mixture was stirred for 12 h, filtered, the filter cake washed with EtOAc (3×300 mL), and the filtrate concentrated under reduced pressure. The concentrate was extracted with EtOAc (3×500 mL) and the combined organic extracts were dried over Na₂SO₄, filtered, and concentrated under reduced pressure to afford (S)-(3-bromo-5-(2-((tert-butoxycarbonyl)amino)-3-methoxy-3-oxopropyl)phenyl)boronic acid (72 g, crude) which was used in the next reaction without further purification. LCMS (ESI) m/z: [M+Na] calcd for C₁₅H₂₁BBrNO₆: 424.0; found 424.1.

Step 3. To a solution of (S)-(3-bromo-5-(2-((tert-butoxycarbonyl)amino)-3-methoxy-3-oxopropyl)phenyl)boronic acid (10.5 g, 26.2 mmol) in MeCN (110 mL) was added NIS (23.5 g, 105 mmol). The resulting mixture was stirred at 80° C. for 12 h, filtered, the filter cake washed with EtOAc (3×300 mL), and the filtrate concentrated under reduced pressure. Purification by reversed phase chromatography afforded methyl (S)-3-(3-bromo-5-iodophenyl)-2-((tert-butoxycarbonyl)amino)propanoate (4.06 g, 32% yield) as a yellow solid. LCMS (ESI) m/z: [M+Na] calcd for C₁₅H₁₉BrINO₄: 508.0; found 507.9.

Step 4. To a solution of methyl (S)-3-(3-bromo-5-iodophenyl)-2-((tert-butoxycarbonyl)amino)propanoate (2.5 g, 5.16 mmol) in DCM (24 mL) at 0° C. was added TFA (8.0 mL). The resulting mixture was stirred for 1 h at room temperature and was then concentrated under reduced pressure to afford methyl (S)-2-amino-3-(3-bromo-5-iodophenyl)propanoate (2.8 g, crude) which was taken directly to the next reaction without further purification. LCMS (ESI) m/z: [M+H] calcd for C₁₀H₁₁BrINO₂: 383.9; found 383.9.

Step 5. To a solution of methyl (S)-2-amino-3-(3-bromo-5-iodophenyl)propanoate (2.8 g, crude) and K₂CO₃ (3.02 g, 21.8 mmol) in THE (30 mL) and H₂O (30 mL) at 0° C. was added 2,5-dioxopyrrolidin-1-yl 2-(trimethylsilyl)ethyl carbonate (2.84 g, 10.9 mmol). The resulting mixture was stirred for 1 h at room temperature and was then quenched at 0° C. by the addition of H₂O. The aqueous mixture was extracted with EtOAc (3×30 mL), and the combined organic extracts were dried over Na₂SO₄, filtered, and concentrated under reduced pressure. Purification by reversed phase chromatography afforded methyl (S)-3-(3-bromo-5-iodophenyl)-2-(((2-(trimethylsilyl)ethoxy)carbonyl)amino)propanoate (2.6 g, 95% yield over 2 steps) as a yellow oil. LCMS (ESI) m/z: [M+H] calcd for C₁₆H₂₃BrINO₄Sᵢ: 528.0; found 528.0.

Step 6. To a solution of methyl (S)-3-(3-bromo-5-iodophenyl)-2-(((2-(trimethylsilyl)ethoxy)carbonyl)amino)propanoate (1.12 g, 2.12 mmol) and tert-butyl (2S,3S)-2-ethynyl-3-(methyl((S)-3-methyl-1-oxo-1-(2-(trimethylsilyl)ethoxy)butan-2-yl)carbamoyl)pyrrolidine-1-carboxylate (960 mg, 2.12 mmol) in DMF (12 mL) at room temperature were added CuI (80 mg, 0.424 mmol), Pd(PPh₃)₄ (490 mg, 0.424 mmol) and Et₃N (1.72 g, 16.9 mmol). The resulting mixture was stirred overnight at room temperature and was then quenched at 0° C. by the addition of H₂O. The aqueous mixture was extracted with EtOAc (3×20 mL), and the combined organic extracts were washed with brine (3×20 mL), dried over Na₂SO₄, filtered, and concentrated under reduced pressure. Purification by reversed phase chromatography tert-butyl (2S,3S)-2-((3-bromo-5-((S)-3-methoxy-3-oxo-2-(((2-(trimethylsilyl)ethoxy)carbonyl)amino)propyl)phenyl)ethynyl)-3-(methyl((S)-3-methyl-1-oxo-1-(2-(trimethylsilyl)ethoxy)butan-2-yl)carbamoyl)pyrrolidine-1-carboxylate (1.7 g, 82% yield) as a yellow oil. LCMS (ESI) m/z: [M+NH₄] calcd for C₃₉H₆₂BrN₃O₉Si₂: 869.4; found 869.4.

Step 7. To a solution of tert-butyl (2S,3S)-2-((3-bromo-5-((S)-3-methoxy-3-oxo-2-(((2-(trimethylsilyl)ethoxy)carbonyl)amino)propyl)phenyl)ethynyl)-3-(methyl((S)-3-methyl-1-oxo-1-(2-(trimethylsilyl)ethoxy)butan-2-yl)carbamoyl)pyrrolidine-1-carboxylate (1.7 g, 1.92 mmol) in toluene (20 mL) at room temperature was added RhCI(PPh₃)₃ (1.7 g, 1.84 mmol). The resulting mixture was stirred overnight at 60° C. under an atmosphere of H₂, filtered, the filter cake washed MeOH (3×30 mL), and the filtrate concentrated under reduced pressure. Purification by reversed phase chromatography afforded tert-butyl (2R,3S)-2-(3-bromo-5-((S)-3-methoxy-3-oxo-2-(((2-(trimethylsilyl)ethoxy)carbonyl)amino)propyl)phenethyl)-3-(methyl((S)-3-methyl-1-oxo-1-(2-(trimethylsilyl)ethoxy)butan-2-yl)carbamoyl)pyrrolidine-1-carboxylate (784 mg, 41% yield) as a brown oil. LCMS (ESI) m/z: [M+ACN+Na] calcd for C₃₉H₆₆BrN₃O₉Si₂: 949.2; found 949.4.

Step 8. To a solution of tert-butyl (2R,3S)-2-(3-bromo-5-((S)-3-methoxy-3-oxo-2-(((2-(trimethylsilyl)ethoxy)carbonyl)amino)propyl)phenethyl)-3-(methyl((S)-3-methyl-1-oxo-1-(2-(trimethylsilyl)ethoxy)butan-2-yl)carbamoyl)pyrrolidine-1-carboxylate (784 mg, 0.883 mmol) in MeCN (8.0 mL) at room temperature was added CsF (3.35 g, 22.1 mmol). The resulting mixture was stirred for 3 h at 60° C., filtered, and the filter cake washed with MeCN (3×15 mL). The filtrate was concentrated under reduced pressure to afford N-((2R,3S)-2-(3-((S)-2-amino-3-methoxy-3-oxopropyl)-5-bromophenethyl)-1-(tert-butoxycarbonyl)pyrrolidine-3-carbonyl)-N-methyl-L-valine (687 mg, crude) which was taken directly to the next reaction without further purification. LCMS (ESI) m/z: [M+H] calcd for C₂₈H₄₂BrN₃O₇: 612.2; found 612.0.

Step 9. To a solution of N-((2R,3S)-2-(3-((S)-2-amino-3-methoxy-3-oxopropyl)-5-bromophenethyl)-1-(tert-butoxycarbonyl)pyrrolidine-3-carbonyl)-N-methyl-L-valine (687 mg, crude) and DIPEA (1.45 g, 11.2 mmol) in DMF (70 mL) at 0° C. was added HATU (426 mg, 1.12 mmol). The resulting mixture was stirred for 1 h at room temperature and was then quenched at 0° C. by the addition of H₂O. The aqueous mixture was extracted with EtOAc (3×70 mL), and the combined organic extracts were washed with brine (3×70 mL), dried over Na₂SO₄, filtered, and concentrated under reduced pressure. Purification by reversed phase chromatography afforded 1¹-(tert-butyl) 6-methyl (1²R,1³S,6S,9S)-4⁵-bromo-9-isopropyl-10-methyl-8,11-dioxo-7,10-diaza-1(2,3)-pyrrolidina-4(1,3)-benzenacycloundecaphane-1¹,6-dicarboxylate (180 mg, 34% yield over 2 steps) as a yellow oil. LCMS (ESI) m/z: [M+H−56] calcd for C₂₈H₄₀BrN₃O₆: 538.2; found 538.1.

Step 10. To a solution of 1¹-(tert-butyl) 6-methyl (1²R,1³S,6S,9S)-4⁵-bromo-9-isopropyl-10-methyl-8,11-dioxo-7,10-diaza-1(2,3)-pyrrolidina-4(1,3)-benzenacycloundecaphane-1¹,6-dicarboxylate (180 mg, 0.303 mmol) in THE (2.0 mL) and H₂O (2.0 mL) stirred at 0° C. was added LiOH·H₂O (25.4 mg, 0.606 mmol). The resulting mixture was stirred for 2 h at room temperature and was then acidified to pH=5 with 1M aq. HCl. The aqueous mixture was extracted with 3:1 vol. DCM/IPA (3×10 mL), dried over Na₂SO₄, filtered, and concentrated under reduced pressure to afford (1²R,1³S,6S,9S)-4⁵-bromo-1¹-(tert-butoxycarbonyl)-9-isopropyl-10-methyl-8,11-dioxo-7,10-diaza-1 (2,3)-pyrrolidina-4(1,3)-benzenacycloundecaphane-6-carboxylic acid (174 mg, crude) which was used in the next reaction without further purification. LCMS (ESI) m/z: [M+H−56] calcd for C₂₇H₃₈BrN₃O₆: 524.2; found 524.2.

Step 11. To a solution of (1²R,1³S,6S,9S)-4⁵-bromo-1¹-(tert-butoxycarbonyl)-9-isopropyl-10-methyl-8,11-dioxo-7,10-diaza-1 (2,3)-pyrrolidina-4(1,3)-benzenacycloundecaphane-6-carboxylic acid (172 mg, crude), methyl (S)-hexahydropyridazine-3-carboxylate (85.4 mg, 0.592 mmol), and DIPEA (382 mg, 2.96 mmol) in DMF (3.0 mL) stirred at 0° C. was added HATU (225 mg, 0.592 mmol). The resulting mixture was stirred for 1 h at room temperature and was then quenched at 0° C. by the addition of H₂O. The aqueous mixture was extracted with EtOAc (3×10 mL), and the combined organic extracts were washed with brine (3×10 mL), dried over Na₂SO₄, filtered, and concentrated under reduced pressure. Purification by reversed phase chromatography afforded tert-butyl (1²R,1³S,6S,9S)-4⁵-bromo-9-isopropyl-6-((S)-3-(methoxycarbonyl)hexahydropyridazine-1-carbonyl)-10-methyl-8,11-dioxo-7,10-diaza-1(2,3)-pyrrolidina-4(1,3)-benzenacycloundecaphane-1¹-carboxylate (143 mg, 67% yield over 2 steps) as a yellow solid. LCMS (ESI) m/z: [M+NH₄] calcd for C₃₃H₄₈BrN₅O₇: 725.7; found 725.4.

Step 12. To a solution of tert-butyl (1²R,1³S,6S,9S)-4⁵-bromo-9-isopropyl-6-((S)-3-(methoxycarbonyl)hexahydropyridazine-1-carbonyl)-10-methyl-8,11-dioxo-7,10-diaza-1(2,3)-pyrrolidina-4(1,3)-benzenacycloundecaphane-1¹-carboxylate (140 mg, 0.198 mmol), (S)-(2-(5-(4-cyclopropylpiperazin-1-yl)-2-(1-methoxyethyl)pyridin-3-yl)-1-ethyl-3-(3-hydroxy-2,2-dimethylpropyl)-1H-indol-5-yl)boronic acid (158 mg, 0.297 mmol), and K₃PO₄ (84.1 mg, 0.396 mmol) in toluene (1.5 mL), dioxane (500 μL), and H₂O (500 μL) stirred at room temperature was added PdCl₂(dtbpf (12.9 mg, 0.020 mmol). The resulting mixture was stirred for 3 h at 70° C. and was then quenched ay 0° C. by the addition of H₂O. The aqueous mixture was extracted with EtOAc (3×10 mL), and the combined organic extracts were dried over Na₂SO₄, filtered, and concentrated under reduced pressure to afford tert-butyl (1²R,1³S,6S,9S)-4⁵-(2-(5-(4-cyclopropylpiperazin-1-yl)-2-((S)-1-methoxyethyl)pyridin-3-yl)-1-ethyl-3-(3-hydroxy-2,2-dimethylpropyl)-1H-indol-5-yl)-9-isopropyl-6-((S)-3-(methoxycarbonyl)hexahydropyridazine-1-carbonyl)-10-methyl-8,11-dioxo-7,10-diaza-1(2,3)-pyrrolidina-4(1,3)-benzenacycloundecaphane-1¹-carboxylate (285 mg, crude) which was used in the next reaction without further purification. LCMS (ESI) m/z: [M+H] calcd for C₆₃H₈₉N₉O₉: 1116.7; found 1116.5.

Step 13. To a solution of tert-butyl (1²R,1³S,6S,9S)-4⁵-(2-(5-(4-cyclopropylpiperazin-1-yl)-2-((S)-1-methoxyethyl)pyridin-3-yl)-1-ethyl-3-(3-hydroxy-2,2-dimethylpropyl)-1H-indol-5-yl)-9-isopropyl-6-((S)-3-(methoxycarbonyl)hexahydropyridazine-1-carbonyl)-10-methyl-8,11-dioxo-7,10-diaza-1(2,3)-pyrrolidina-4(1,3)-benzenacycloundecaphane-1¹-carboxylate (283 mg, crude) in THE (3.0 mL) and H₂O (3.0 mL) stirred at 0° C. was added LiOH·H₂O (21.3 mg, 0.506 mmol). The resulting mixture was stirred for 2 h at room temperature then acidified to pH=5 with 1M aq. HCl. The aqueous mixture was extracted with 3:1 vol. DCM/IPA (3×10 mL), and the combined organic extracts were dried with Na₂SO₄, filtered, and concentrated under reduced pressure to afford (S)-1-((1²R,1³S,6S,9S)-1¹-(tert-butoxycarbonyl)-4⁵-(2-(5-(4-cyclopropylpiperazin-1-yl)-2-((S)-1-methoxyethyl)pyridin-3-yl)-1-ethyl-3-(3-hydroxy-2,2-dimethylpropyl)-1H-indol-5-yl)-9-isopropyl-10-methyl-8,11-dioxo-7,10-diaza-1(2,3)-pyrrolidina-4(1,3)-benzenacycloundecaphane-6-carbonyl)hexahydropyridazine-3-carboxylic acid (180 mg, crude) which was used in the next reaction without further purification. LCMS (ESI) m/z: [M+H] calcd for C₆₂H₈₇N₉O₉: 1102.7; found 1102.5.

Step 14. To a solution of (S)-1-((1²R,1³S,6S,9S)-1¹-(tert-butoxycarbonyl)-4⁵-(2-(5-(4-cyclopropylpiperazin-1-yl)-2-((S)-1-methoxyethyl)pyridin-3-yl)-1-ethyl-3-(3-hydroxy-2,2-dimethylpropyl)-1H-indol-5-yl)-9-isopropyl-10-methyl-8,11-dioxo-7,10-diaza-1(2,3)-pyrrolidina-4(1,3)-benzenacycloundecaphane-6-carbonyl)hexahydropyridazine-3-carboxylic acid (175 mg, crude), and DIPEA (615 mg, 4.77 mmol) in DCM (18 mL) at 0° C. were added EDCI (609 mg, 3.18 mmol) and HOBT (214 mg, 1.59 mmol). The resulting mixture was stirred overnight at room temperature then acidified to pH=5 with 1M aq. HCl. The aqueous mixture was extracted with 3:1 vol. DCM/IPA (3×20 mL), and the combined organic extracts were dried with Na₂SO₄, filtered, and concentrated under reduced pressure. Purification by normal phase prep-TLC afforded tert-butyl (3aS,6S,9S,15S,32aR)-21-(5-(4-cyclopropylpiperazin-1-yl)-2-((S)-1-methoxyethyl)pyridin-3-yl)-22-ethyl-6-isopropyl-5,19,19-trimethyl-4,7,10,16-tetraoxo-2,3,3a,5,6,7,8,9,10,13,14,15,16,18,19,20,22,31,32,32a-icosahydro-12H-11,15-epimino-23,25-etheno-9,28-methano-26,30-(metheno)dipyrrolo[3,2-o:3′,4′-z][1]oxa[7,10,13]triazacyclotriacontine-1(4H)-carboxylate (100 mg, 47% yield over 3 steps) as a yellow solid. LCMS (ESI) m/z: [M+H] calcd for C₆₂H₈₅N₉O₈: 1084.7; found 1084.9.

Step 15. To a solution of tert-butyl (3aS,6S,9S,15S,32aR)-21-(5-(4-cyclopropylpiperazin-1-yl)-2-((S)-1-methoxyethyl)pyridin-3-yl)-22-ethyl-6-isopropyl-5,19,19-trimethyl-4,7,10,16-tetraoxo-2,3,3a,5,6,7,8,9,10,13,14,15,16,18,19,20,22,31,32,32a-icosahydro-12H-11,15-epimino-23,25-etheno-9,28-methano-26,30-(metheno)dipyrrolo[3,2-o:3′,4′-z][1]oxa[7,10,13]triazacyclotriacontine-1(4H)-carboxylate (98 mg, 0.090 mmol) in DCM (1.0 mL) stirred at 0° C. was added TFA (200 μL). The resulting mixture was stirred for 1 h at room temperature then was basified to pH 8=with sat. aq. NaHCO₃. The aqueous mixture was extracted with 3:1 vol DCM/IPA (3×10 mL), and the combined organic extracts were dried with Na₂SO₄, filtered, and concentrated under reduced pressure to afford (3aS,6S,9S,15S,32aR)-21-(5-(4-cyclopropylpiperazin-1-yl)-2-((S)-1-methoxyethyl)pyridin-3-yl)-22-ethyl-6-isopropyl-5,19,19-trimethyl-2,3,3a,5,6,8,9,14,15,18,19,20,22,31,32,32a-hexadecahydro-12H-11,15-epimino-23,25-etheno-9,28-methano-26,30-(metheno)dipyrrolo[3,2-o:3′,4′-z][1]oxa[7,10,13]triazacyclotriacontine-4,7,10,16(1H,13H)-tetraone (88 mg, crude) which was used in the next reaction without further purification. LCMS (ESI) m/z: [M+H] calcd for C₅₇H₇₇N₉O₆: 984.6; found 984.8.

Step 16. To a solution of (3aS,6S,9S,15S,32aR)-21-(5-(4-cyclopropylpiperazin-1-yl)-2-((S)-1-methoxyethyl)pyridin-3-yl)-22-ethyl-6-isopropyl-5,19,19-trimethyl-2,3,3a,5,6,8,9,14,15,18,19,20,22,31,32,32a-hexadecahydro-12H-11,15-epimino-23,25-etheno-9,28-methano-26,30-(metheno)dipyrrolo[3,2-o:3′,4′-z][1]oxa[7,10,13]triazacyclotriacontine-4,7,10,16(1H,13H)-tetraone (86 mg, crude), DIPEA (113 mg, 0.870 mmol), and propanoic acid (19.4 mg, 0.261 mmol) in DMF (2.0 mL) stirred at 0° C. was added COMU (74.8 mg, 0.174 mmol). The resulting mixture was stirred for 1 h at room temperature and was then quenched at 0° C. by the addition of H₂O. The aqueous mixture was extracted with EtOAc (3×10 mL), and the combined organic extracts were washed with brine (3×10 mL), dried over Na₂SO₄, filtered, and concentrated under reduced pressure. Purification by reversed phase chromatography afforded (3aS,6S,9S,15S,32aR)-21-(5-(4-cyclopropylpiperazin-1-yl)-2-((S)-1-methoxyethyl)pyridin-3-yl)-22-ethyl-6-isopropyl-5,19,19-trimethyl-1-propionyl-2,3,3a,5,6,8,9,14,15,18,19,20,22,31,32,32a-hexadecahydro-12H-11,15-epimino-23,25-etheno-9,28-methano-26,30-(metheno)dipyrrolo[3,2-o:3′,4′-z][1]oxa[7,10,13]triazacyclotriacontine-4,7,10,16(1H,13H)-tetraone (25.8 mg, 28% yield over 2 steps). LCMS (ESI) m/z: [M+H] calcd for C₆₀H₈₁N₉O₇: 1040.6; found 1040.5; ¹H NMR (400 MHz, DMSO-d₆) δ 8.22 (s, 1H), 7.98-7.60 (m, 1H), 7.60-7.42 (m, 1H), 7.42-7.10 (m, 3H), 7.07-6.95 (m, 1H), 6.72-6.07 (m, 2H), 5.33-4.89 (m, 2H), 4.40-4.23 (m, 1H), 4.07 (br s, 2H), 3.90-3.37 (m, 6H), 3.08-2.94 (m, 4H), 2.78-2.19 (m, 17H), 2.09-1.70 (m, 7H), 1.60-1.29 (m, 7H), 1.20-0.89 (m, 8H), 0.87-0.30 (m, 15H), 0.21 (d, J=11.3 Hz, 3H).

### Example A285. Synthesis of (3aS,6S,9S,15S,32aS)-21-(5-(4-(2,2-difluoroethyl)piperazin-1-yl)-2-((S)-1-methoxyethyl)pyridin-3-yl)-22-ethyl-1-(2-fluoro-2-methylpropanoyl)-6-isopropyl-5,19,19-trimethyl-3,3a,5,6,8,9,14,15,18,19,20,22,32,32a-tetradecahydro-1H,12H-11,15-epimino-23,25-etheno-9,28-methano-26,30-(metheno)dipyrrolo[2,3-c:3′,4′-v][1,18]dioxa[6,9,12]triazacyclotriacontine-4,7,10,16(2H,13H)-tetraone

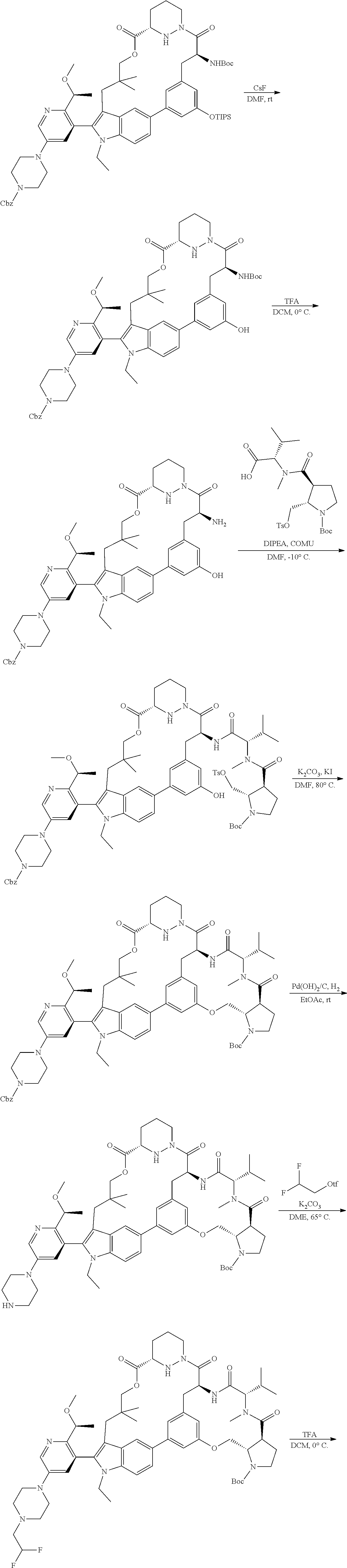

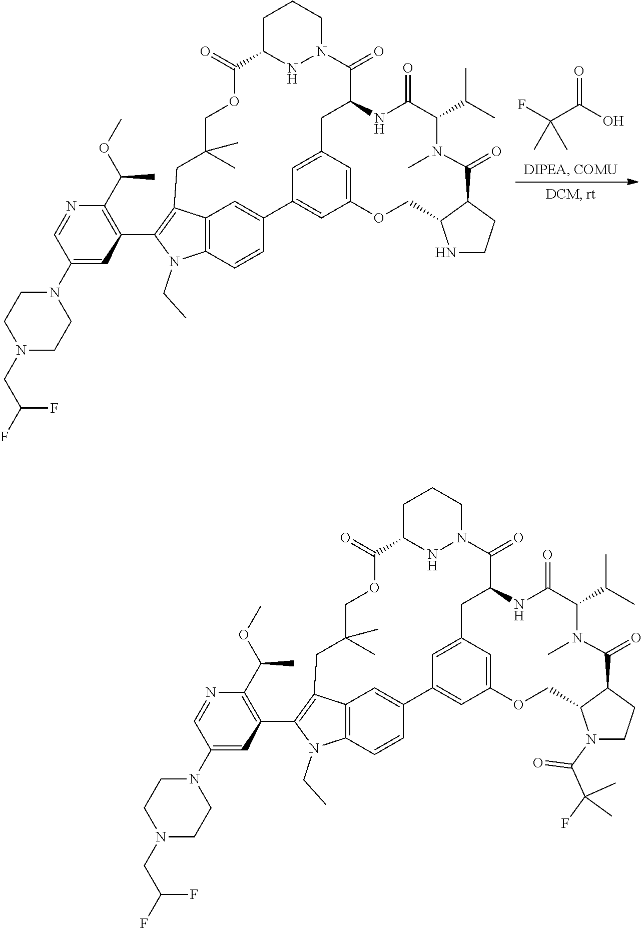

Step 1. To a solution of benzyl 4-(5-((6³S,4S)-4-((tert-butoxycarbonyl)amino)-1¹-ethyl-10,10-dimethyl-5,7-dioxo-2⁵-((triisopropylsilyl)oxy)-6¹,6²,6³,6⁴,6⁵,6⁶-hexahydro-1¹H-8-oxa-1(5,3)-indola-6(1,3)-pyridazina-2(1,3)-benzenacycloundecaphane-1²-yl)-6-((S)-1-methoxyethyl)pyridin-3-yl)piperazine-1-carboxylate (40.0 g, 35.9 mmol) in DMF (200 mL) was added CsF (16.4 g, 108 mmol) at 0° C. The resulting mixture was stirred for 4 h at room temperature and was then quenched by the addition of H₂O (200 mL). The aqueous mixture was extracted with EtOAc (3×200 mL), and the combined organic extracts were washed with brine (3×100 mL), dried over Na₂SO₄, filtered, and concentrated under reduced pressure. The residue was purified by reversed phase chromatography to give benzyl 4-(5-((6³S,4S)-4-((tert-butoxycarbonyl)amino)-1¹-ethyl-2⁵-hydroxy-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-1²-yl)-6-((S)-1-methoxyethyl)pyridin-3-yl)piperazine-1-carboxylate (22.0 g, 64% yield) as a yellow solid. LCMS (ESI) m/z: [M+H] calcd for C₅₄H₆₇N₇O₉: 958.5; found 958.5.

Step 2. To a solution of benzyl 4-(5-((6³S,4S)-4-((tert-butoxycarbonyl)amino)-1¹-ethyl-2⁵-hydroxy-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)- benzenacycloundecaphane-1²-yl)-6-((S)-1-methoxyethyl)pyridin-3-yl)piperazine-1-carboxylate (12.0 g, 12.5 mmol) in DCM (90 mL) was added TFA (30 mL) dropwise at 0° C. The resulting mixture was stirred for 1 h at room temperature, concentrated under reduced pressure, and diluted with H₂O (50 mL). The aqueous mixture was basified to pH 8 with sat. aq. NaHCO₃ and extracted with DCM (3×40 mL). The combined organic extracts were dried over Na₂SO₄, filtered, and concentrated under reduced pressure to give benzyl 4-(5-((6³S,4S)-4-amino-1′-ethyl-2⁵-hydroxy-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-1²-yl)-6-((S)-1-methoxyethyl)pyridin-3-yl)piperazine-1-carboxylate (10.4 g, crude), which was taken directly to the next reaction without further purification. LCMS (ESI) m/z: [M+H] calcd for C₄₉H₅₉N₇O₇: 858.4; found 858.2.

Step 3. To a solution of benzyl 4-(5-((6³S,4S)-4-amino-1′-ethyl-2⁵-hydroxy-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-1²-yl)-6-((S)-1-methoxyethyl)pyridin-3-yl)piperazine-1-carboxylate (10.4 g, crude) and (2S)-2-{1-[(2S,3S)-1-(tert-butoxycarbonyl)-2-{[(4-methylbenzenesulfonyl)oxy]methyl}pyrrolidin-3-yl]-N-methylformamido}-3-methylbutanoic acid (8.08 g, 15.8 mmol) in DMF (100 mL) were added DIPEA (62.7 g, 485 mmol) and COMU (6.75 g, 15.8 mmol) at −10° C. The resulting mixture was stirred for 1 h at −10° C. and was then quenched by the addition of H₂O (30 mL) at 0° C. The aqueous mixture was acidified to pH 7 with 1 M aq. HCl and extracted with EtOAc (3×100 mL). The combined organic extracts were washed with brine (3×300 mL), dried over anhydrous Na₂SO₄, filtered, concentrated under reduced pressure, and purified by normal phase chromatography to give benzyl 4-(5-((6³S,4S)-4-((S)-2-((2S,3S)-1-(tert-butoxycarbonyl)-N-methyl-2-((tosyloxy)methyl)pyrrolidine-3-carboxamido)-3-methylbutanamido)-1¹-ethyl-2⁵-hydroxy-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-1²-yl)-6-((S)-1-methoxyethyl)pyridin-3-yl)piperazine-1-carboxylate (15.7 g, 93% yield over 2 steps) as a yellow solid. LCMS (ESI) m/z: [M+H] calcd for C₇₃H₉₃N₉O₁₄S: 1352.7; found 1353.3.

Step 4. To a solution of benzyl 4-(5-((6³S,4S)-4-((S)-2-((2S,3S)-1-(tert-butoxycarbonyl)-N-methyl-2-((tosyloxy)methyl)pyrrolidine-3-carboxamido)-3-methylbutanamido)-1¹-ethyl-2⁵-hydroxy-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-1²-yl)-6-((S)-1-methoxyethyl)pyridin-3-yl)piperazine-1-carboxylate (15.6 g, 11.5 mmol) in DMF (1.5 L) were added K₂CO₃ (15.9 g, 115 mmol) and KI (1.91 g, 11.5 mmol) at room temperature. The resulting mixture was stirred for 3 h at 80° C. under and atmosphere of N₂ and was then quenched by the addition of cold H₂O (3 L) at 0° C. The aqueous mixture was extracted with EtOAc (3×3 L), and the combined organic layers were washed with brine (3×3 L), dried over Na₂SO₄, filtered, and concentrated under reduced pressure. The crude residue was purified by reversed phase chromatography to give tert-butyl (3aS,6S,9S,15S,32aS)-21-(5-(4-((benzyloxy)carbonyl)piperazin-1-yl)-2-((S)-1-methoxyethyl)pyridin-3-yl)-22-ethyl-6-isopropyl-5,19,19-trimethyl-4,7,10,16-tetraoxo-2,3,3a,4,5,6,7,8,9,10,13,14,15,16,18,19,20,22,32,32a-icosahydro-1H,12H-11,15-epimino-23,25-etheno-9,28-methano-26,30-(metheno)dipyrrolo[2,3-c:3′,4′-v][1,18]dioxa[6,9,12]triazacyclotriacontine-1-carboxylate (8.2 g, 60% yield) as a yellow solid. LCMS (ESI) m/z: [M+H+NH₄] calcd for C₅₈H₇₈N₉O₉: 599.3; found 599.5.

Step 5. To a solution of tert-butyl (3aS,6S,9S,15S,32aS)-22-ethyl-6-isopropyl-21-(2-((S)-1-methoxyethyl)-5-(piperazin-1-yl)pyridin-3-yl)-5,19,19-trimethyl-4,7,10,16-tetraoxo-2,3,3a,4,5,6,7,8,9,10,13,14,15,16,18,19,20,22,32,32a-icosahydro-1H,12H-11,15-epimino-23,25-etheno-9,28-methano-26,30-(metheno)dipyrrolo[2,3-c:3′,4′-v][1,18]dioxa[6,9,12]triazacyclotriacontine-1-carboxylate (2.00 g, 1.69 mmol) in EtOAc (20 mL) at room temperature was added Pd(OH)₂/C (0.59 g) under an atmosphere of N₂. The reaction mixture was stirred at room temperature for 3 h under an atmosphere of H₂, filtered, and concentrated under reduced pressure to give tert-butyl (3aS,6S,9S,15S,32aS)-22-ethyl-6-isopropyl-21-(2-((S)-1-methoxyethyl)-5-(piperazin-1-yl)pyridin-3-yl)-5,19,19-trimethyl-4,7,10,16-tetraoxo-2,3,3a,4,5,6,7,8,9,10,13,14,15,16,18,19,20,22,32,32a-icosahydro-1H,12H-11,15-epimino-23,25-etheno-9,28-methano-26,30-(metheno)dipyrrolo[2,3-c:3′,4′-v][1,18]dioxa[6,9,12]triazacyclotriacontine-1-carboxylate (1.58 g, crude), which was used without further purification. LCMS (ESI) m/z: [M+H] calcd for C₅₈H₇₉N₉O₉: 1046.6; found 1047.2.

Step 6. To a solution of tert-butyl (3aS,6S,9S,15S,32aS)-22-ethyl-6-isopropyl-21-(2-((S)-1-methoxyethyl)-5-(piperazin-1-yl)pyridin-3-yl)-5,19,19-trimethyl-4,7,10,16-tetraoxo-2,3,3a,4,5,6,7,8,9,10,13,14,15,16,18,19,20,22,32,32a-icosahydro-1H,12H-11,15-epimino-23,25-etheno-9,28-methano-26,30-(metheno)dipyrrolo[2,3-c:3′,4′-v][1,18]dioxa[6,9,12]triazacyclotriacontine-1-carboxylate (700 mg, crude) and K₂CO₃ (279 mg, 2.01 mmol) in DME (7 mL) was added 2,2-difluoroethyl trifluoromethanesulfonate (172 mg, 0.803 mmol) at room temperature. The reaction mixture was stirred for 2 h at 65° C. and was then quenched by the addition of H₂O (30 mL). The aqueous mixture was extracted with EtOAc (2×30 mL) and the combined organic extracts were dried over Na₂SO₄, filtered, concentrated under reduced pressure, and purified by normal phase chromatography to give tert-butyl (3aS,6S,9S,15S,32aS)-21-(5-(4-(2,2-difluoroethyl)piperazin-1-yl)-2-((S)-1-methoxyethyl)pyridin-3-yl)-22-ethyl-6-isopropyl-5,19,19-trimethyl-4,7,10,16-tetraoxo-2,3,3a,4,5,6,7,8,9,10,13,14,15,16,18,19,20,22,32,32a-icosahydro-1H,12H-11,15-epimino-23,25-etheno-9,28-methano-26,30-(metheno)dipyrrolo[2,3-c:3′,4′-v][1,18]dioxa[6,9,12]triazacyclotriacontine-1-carboxylate (640 mg, 77% over 2 steps) as a white solid. LCMS (ESI) m/z: [M+H] calcd for C₆₀H₈₁F₂N₉O₉: 1110.6; found 1110.7.

Step 7. To a solution of tert-butyl (3aS,6S,9S,15S,32aS)-21-(5-(4-(2,2-difluoroethyl)piperazin-1-yl)-2-((S)-1-methoxyethyl)pyridin-3-yl)-22-ethyl-6-isopropyl-5,19,19-trimethyl-4,7,10,16-tetraoxo-2,3,3a,4,5,6,7,8,9,10,13,14,15,16,18,19,20,22,32,32a-icosahydro-1H,12H-11,15-epimino-23,25-etheno-9,28-methano-26,30-(metheno)dipyrrolo[2,3-c:3′,4′-v][1,18]dioxa[6,9,12]triazacyclotriacontine-1-carboxylate (640 mg, 0.576 mmol) in DCM (6 mL) was added TFA (2.0 mL) dropwise at 0° C. The reaction mixture was stirred for 1 h at 0° C. and was then concentrated under reduced pressure. The resulting mixture was basified to pH 6 with sat. aq. NaHCO₃ (20 mL), extracted with EtOAc (2×20 mL), and the combined organic extracts dried over Na₂SO₄, filtered, and concentrated under reduced pressure to give (3aS,6S,9S,15S,32aS)-21-(5-(4-(2,2-difluoroethyl)piperazin-1-yl)-2-((S)-1-methoxyethyl)pyridin-3-yl)-22-ethyl-6-isopropyl-5,19,19-trimethyl-3,3a,5,6,8,9,14,15,18,19,20,22,32,32a-tetradecahydro-1H,12H-11,15-epimino-23,25-etheno-9,28-methano-26,30-(metheno)dipyrrolo[2,3-c:3′,4′-v][1,18]dioxa[6,9,12]triazacyclotriacontine-4,7,10,16(2H,13H)-tetraone (520 mg, crude), which was used without further purification. LCMS (ESI) m/z: [M+H] calcd for C₅₅H₇₃F₂N₉O₇: 1010.6; found 1010.6.

Step 8. To a solution of (3aS,6S,9S,15S,32aS)-21-(5-(4-(2,2-difluoroethyl)piperazin-1-yl)-2-((S)-1-methoxyethyl)pyridin-3-yl)-22-ethyl-6-isopropyl-5,19,19-trimethyl-3,3a,5,6,8,9,14,15,18,19,20,22,32,32a-tetradecahydro-1H,12H-11,15-epimino-23,25-etheno-9,28-methano-26,30-(metheno)dipyrrolo[2,3-c:3′,4′-v][1,18]dioxa[6,9,12]triazacyclotriacontine-4,7,10,16(2H,13H)-tetraone (350 mg, crude) in DCM (5 mL) were added 2-fluoro-2-methylpropanoic acid (110 mg, 1.04 mmol), DIPEA (448 mg, 3.46 mmol) and COMU (297 mg, 0.692 mmol) at 0° C. The reaction mixture was stirred for 2 h at 0° C. and was then quenched by the addition of sat. aq. NH₄Cl (20 mL) and extracted with DCM (2×20 mL). The combined organic extracts were dried over anhydrous Na₂SO₄, filtered, and concentrated under reduced pressure. The residue was purified by reversed phase chromatography to give (3aS,6S,9S,15S,32aS)-21-(5-(4-(2,2-difluoroethyl)piperazin-1-yl)-2-((S)-1-methoxyethyl)pyridin-3-yl)-22-ethyl-1-(2-fluoro-2-methylpropanoyl)-6-isopropyl-5,19,19-trimethyl-3,3a,5,6,8,9,14,15,18,19,20,22,32,32a-tetradecahydro-1H,12H-11,15-epimino-23,25-etheno-9,28-methano-26,30-(metheno)dipyrrolo[2,3-c:3′,4′-v][1,18]dioxa[6,9,12]triazacyclotriacontine-4,7,10,16(2H,13H)-tetraone (163 mg, 38% yield over 2 steps) as a white solid. LCMS (ESI) m/z: [M+H] calcd for C₅₉H₇₈F₃N₉O₈: 1098.6; found 1098.7; ¹H NMR (400 MHz, DMSO-d₆) δ 8.47 (d, J=2.8 Hz, 1H), 8.01 (s, 1H), 7.77-7.68 (m, 1H), 7.57 (t, J=8.1 Hz, 1H), 7.48-7.17 (m, 3H), 6.82-6.62 (s, 2H), 6.32-6.04 (s, 2H), 5.40 (d, J=12.3 Hz, 1H), 5.23 (t, J=9.5 Hz, 1H), 5.13 (s, 1H), 5.05-4.97 (m, 1H), 4.73-4.66 (m, 1H), 4.49-4.05 (m, 7H) 3.93-3.80 (m, 1H), 3.70-3.55 (m, 3H), 3.51-3.41 (m, 2H), 3.29-3.15 (m, 4H), 3.03 (s, 2H), 2.92-2.81 (m, 2H), 2.81-2.65 (m, 8H), 2.39 (s, 2H), 2.24 (br s, 2H), 2.10-1.98 (m, 2H), 1.93-1.80 (m, 2H), 1.68 (d, J=11.2 Hz, 1H), 1.61-1.55 (m, 4H), 1.49 (s, 1H), 1.44 (s, 1H), 1.40-1.33 (m, 4H), 1.32-1.21 (m, 2H), 1.01 (t, J=7.0 Hz, 2H), 0.94 (s, 1H), 0.87 (d, J=6.3 Hz, 1H), 0.83-0.71 (m, 4H), 0.68 (d, J=6.7 Hz, 2H), 0.60 (s, 2H), 0.47 (s, 1H).

### Example A310. Synthesis of (3aS,6S,9S,15S,32aS)-21-(5-(4-cyclopropylpiperazin-1-yl)-2-((S)-1-meth oxyethyl)pyridin-3-yl)-22-ethyl-1-(2-fluoro-2-methylpropanoyl)-6-isopropyl-5,19,19-trimethyl-3,3a,5,6,8,9,14,15,18,19,20,22,32,32a-tetradecahydro-1H,12H-11,15-epimino-23,25-etheno-9,28-methano-26,30-(metheno)dipyrrolo[2,3-c:3′,4′-v][1,18]dioxa[6,9,12,24]tetraazacyclotriacontine-4,7,10,16(2H,13H)-tetraone

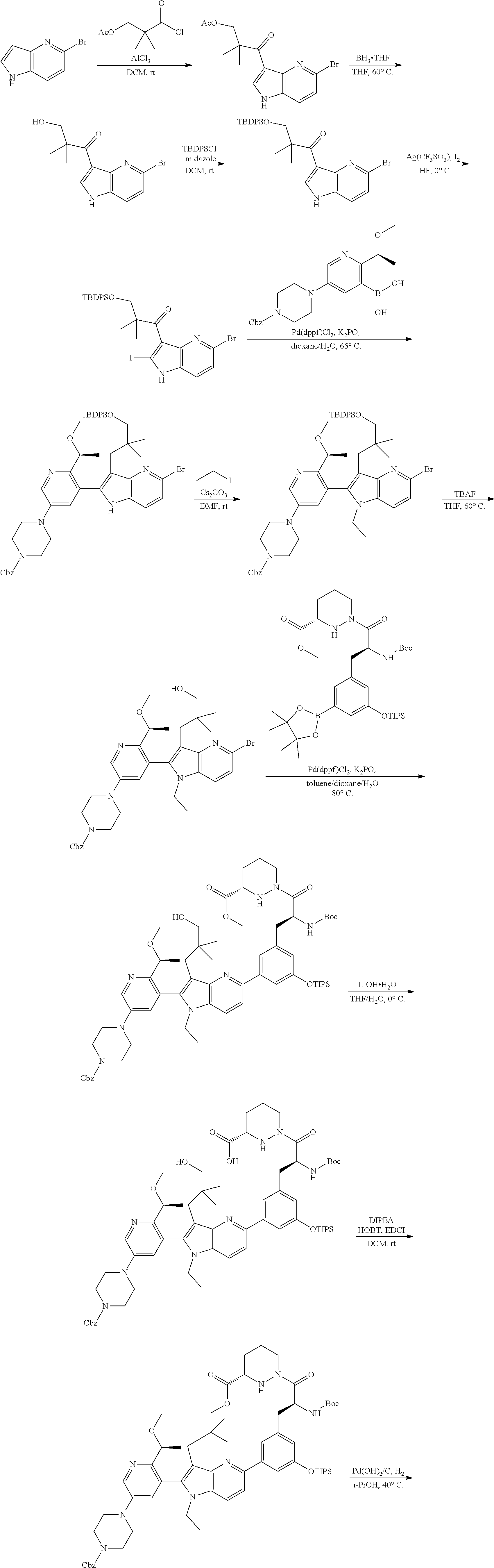

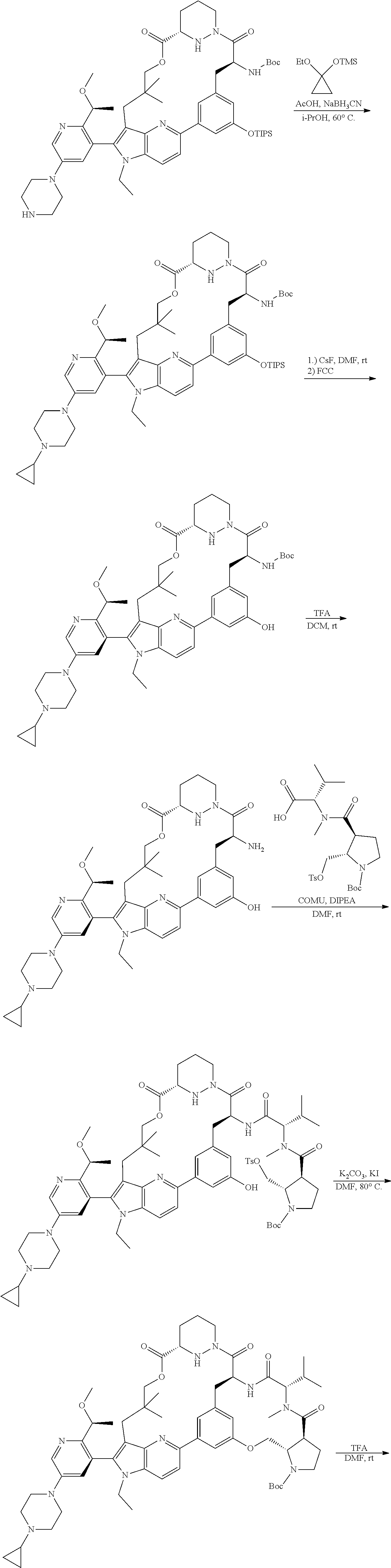

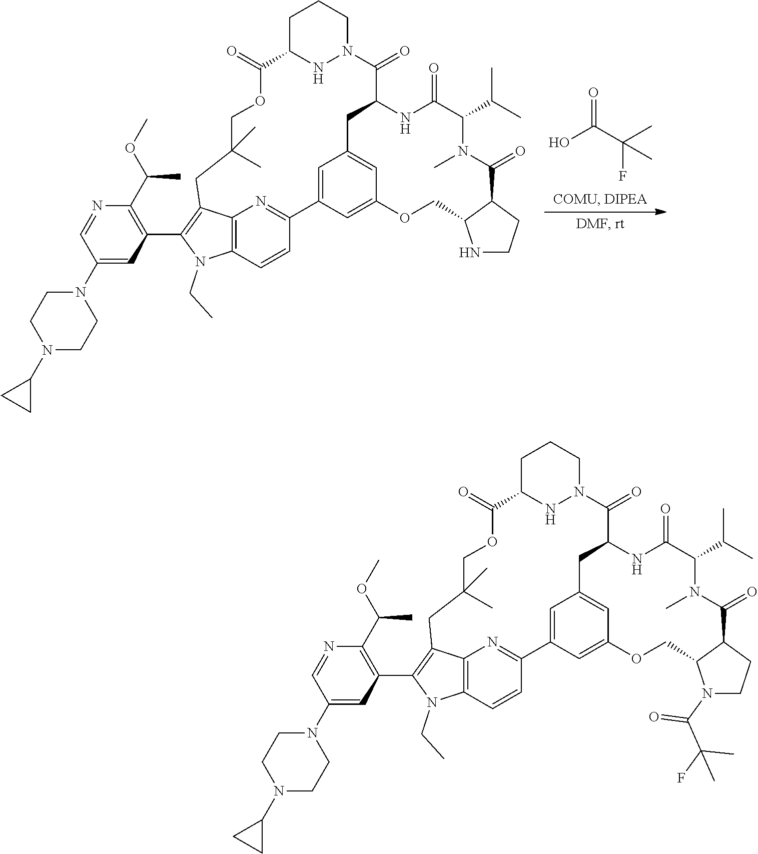

Step 1. To a solution of 5-bromo-1H-pyrrolo[3,2-b]pyridine (20.0 g, 102 mmol) and AlCl₃ (94.7 g, 711 mmol) in DCM (500 mL) was added 3-chloro-2,2-dimethyl-3-oxopropyl acetate (21.8 g, 122 mmol) at 0° C. under an atmosphere of N₂. The reaction mixture was stirred at room temperature for 16 h and was then quenched at 0° C. by the addition of H₂O (300 mL). The resulting mixture was filtered, and the filter cake was washed with DCM (3×100 mL). The filtrate was extracted with DCM (3×200 mL) and the combined organic extracts were dried over Na₂SO₄, filtered, and concentrated under reduced pressure to give 3-[5-bromo-1H-pyrrolo[3,2-b]pyridin-3-yl]-2,2-dimethyl-3-oxopropyl acetate (25.0 g, crude), which was used without further purification. LCMS (ESI) m/z: [M+H] calcd for C₁₄H₁₅BrN₂O₃: 339.0; found 338.9.

Step 2. To a solution of 3-{5-bromo-1H-pyrrolo[3,2-b]pyridin-3-yl}-2,2-dimethyl-3-oxopropyl acetate (25.0 g, crude) in THE (25 mL) was added BH₃·THF (250 mL) dropwise at 0° C. under an atmosphere of N₂. The reaction mixture was stirred at 60° C. for 16 h and was then concentrated under reduced pressure to yield 3-[5-bromo-1H-pyrrolo[3,2-b]pyridin-3-yl]-2,2-dimethylpropan-1-ol (20.0 g, crude), which was used without further purification. LCMS (ESI) m/z: [M+H] calcd for C₁₂H₁₅BrN₂O: 283.0; found 282.8.

Step 3. To a solution of 3-{5-bromo-1H-pyrrolo[3,2-b]pyridin-3-yl}-2,2-dimethylpropan-1-ol (20.0 g, crude) and imidazole (9.62 g, 141 mmol) in DCM (400 mL) was added TBDPSCI (29.1 g, 106 mmol) at 0° C. The reaction mixture was stirred for 16 h at rt and was then quenched by the addition of H₂O (300 mL). The aqueous layer was extracted with DCM (3×200 mL) and the combined organic extracts were dried over Na₂SO₄, filtered, and concentrated under reduced pressure. The residue was purified by normal phase chromatography to give 5-bromo-3-{3-[(tert-butyldiphenylsilyl)oxy]-2,2-dimethylpropyl}-1H-pyrrolo[3,2-b]pyridine (24.0 g, 38% yield over 3 steps) as a yellow solid. LCMS (ESI) m/z: [M+H] calcd for C₂₈H₃₃BrN₂OSi: 521.2; found 521.0.

Step 4. To a solution of 5-bromo-3-{3-[(tert-butyldiphenylsilyl)oxy]-2,2-dimethylpropyl}-1H-pyrrolo[3,2-b]pyridine (10.0 g, 19.2 mmol) and Ag(CF₃SO₃) (6.40 g, 24.9 mmol) in THE (50 mL) was added a solution of 12 (1.70 g, 6.71 mmol) in THE (5 mL) at 0° C. The reaction mixture was stirred for 2 h at 0° C. and was then quenched by the addition of H₂O (40 mL) and EtOAc (40 mL). The resulting mixture was filtered, and the filter cake was washed with EtOAc (3×30 mL). The filtrate was then extracted with EtOAc (2×50 mL) and the combined organic extracts were dried over Na₂SO₄, filtered, concentrated under reduced pressure, and purified by normal phase chromatography to give 5-bromo-3-[3-[(tert-butyldiphenylsilyl)oxy]-2,2-dimethylpropyl]-2-iodo-1H-pyrrolo[3,2-b]pyridine (10.0 g, 64% yield) as a yellow solid. LCMS (ESI) m/z: [M+H] calcd for C₂₈H₃₂BrIN₂OSi: 647.1; found 647.0.

Step 5. To a solution of 5-bromo-3-[3-[(tert-butyldiphenylsilyl)oxy]-2,2-dimethylpropyl]-2-iodo-1H-pyrrolo[3,2-b]pyridine (10.0 g, 15.4 mmol) and 5-{4-[(benzyloxy)carbonyl]piperazin-1-yl}-2-[(1S)-1-methoxyethyl]pyridin-3-ylboronic acid (7.40 g, 18.5 mmol) in dioxane (50 mL) and H₂O (5 mL) were added K₃PO₄ (8.20 g, 38.6 mmol) and Pd(dppf)Cl₂·CH₂Cl₂ (1.26 g, 1.55 mmol) at room temperature. The reaction mixture was stirred for 1.5 h at 65° C. and was then quenched by the addition of H₂O (60 mL). The aqueous mixture was extracted with EtOAc (3×50 mL) and the combined organic extracts were dried over anhydrous Na₂SO₄, filtered, concentrated under reduced pressure, and purified by normal phase chromatography to give benzyl 4-[5-(5-bromo-3-{3-[(tert-butyldiphenylsilyl)oxy]-2,2-dimethylpropyl}-1H-pyrrolo[3,2-b]pyridin-2-yl)-6-[(1S)-1-methoxyethyl]pyridin-3-yl]piperazine-1-carboxylate (6.0 g, 44% yield) as a yellow solid. LCMS (ESI) m/z: [M+H] calcd for C₄₈H₅₆BrN₅O₄Si: 874.3; found 874.3.

Step 6. To a solution of benzyl 4-[5-(5-bromo-3-{3-[(tert-butyldiphenylsilyl)oxy]-2,2-dimethylpropyl}-1H-pyrrolo[3,2-b]pyridin-2-yl)-6-[(1S)-1-methoxyethyl]pyridin-3-yl]piperazine-1-carboxylate (4.00 g, 4.57 mmol) and Cs₂CO₃ (2.98 g, 9.14 mmol) in DMF (40 mL) was added ethyl iodide (1.43 g, 9.14 mmol) at 0° C. The reaction mixture was stirred at room temperature for 2 h and was then quenched by the addition of H₂O (40 mL). The aqueous mixture was extracted with EtOAc (3×40 mL) and the combined organic extracts were washed with brine (3×30 mL), dried over Na₂SO₄, filtered, and concentrated under reduced pressure to give benzyl 4-[5-(5-bromo-3-{3-[(tert-butyldiphenylsilyl)oxy]-2,2-dimethylpropyl}-1-ethylpyrrolo[3,2-b]pyridin-2-yl)-6-[(1 S)-1-methoxyethyl]pyridin-3-yl]piperazine-1-carboxylate (4.2 g, crude), which was used without further purification. LCMS (ESI) m/z: [M+H] calcd for C₅₀H₆₀BrN₅O₄Si: 902.4; found 902.1.

Step 7. To a solution of benzyl 4-[5-(5-bromo-3-{3-[(tert-butyldiphenylsilyl)oxy]-2,2-dimethylpropyl}-1-ethylpyrrolo[3,2-b]pyridin-2-yl)-6-[(1 S)-1-methoxyethyl]pyridin-3-yl]piperazine-1-carboxylate (4.0 g, crude) in THE (10 mL) was added a solution of TBAF in THE (40 mL, 40 mmol) at room temperature. The reaction mixture was stirred overnight at 60° C. and was then quenched by the addition of H₂O (50 mL). The aqueous mixture was extracted with EtOAc (3×40 mL) and the combined organic extracts were dried over Na₂SO₄, filtered, concentrated under reduced pressure, and purified by normal phase chromatography to give benzyl 4-{5-[5-bromo-1-ethyl-3-(3-hydroxy-2,2-dimethylpropyl)pyrrolo[3,2-b]pyridin-2-yl]-6-[(1 S)-1-methoxyethyl]pyridin-3-yl}piperazine-1-carboxylate (1.40 g, 48% yield over 2 steps) as a yellow solid. LCMS (ESI) m/z: [M+H] calcd for C₃₄H₄₂BrN₅O₄: 664.2; found 664.2.

Step 8. To a solution of benzyl 4-{5-[5-bromo-1-ethyl-3-(3-hydroxy-2,2-dimethylpropyl)pyrrolo[3,2-b]pyridin-2-yl]-6-[(1 S)-1-methoxyethyl]pyridin-3-yl}piperazine-1-carboxylate (1.40 g, 2.11 mmol) and methyl (3S)-1-[(2S)-2-[(tert-butoxycarbonyl)amino]-3-[3-(4,4,5,5-tetramethyl-1,3,2-dioxaborolan-2-yl)-5-[(triisopropylsilyl)oxy]phenyl]propanoyl]-1,2-diazinane-3-carboxylate (1.74 g, 2.53 mmol) in toluene (18 mL), dioxane (6 mL), H₂O (6 mL) were added K₃PO₄ (890 mg, 4.21 mmol) and Pd(dppf)Cl₂·CH₂C₁₂ (340 mg, 0.421 mmol) at room temperature. The reaction mixture was stirred for 3 h at 80° C. under an atmosphere of N₂ and was then filtered. The filter cake was washed with EtOAc (3×30 mL) and the filtrate was concentrated under reduced pressure to give benzyl 4-[5-(5-{3-[(2S)-2-[(tert-butoxycarbonyl)amino]-3-[(3S)-3-(methoxycarbonyl)-1,2-diazinan-1-yl]-3-oxopropyl]-5-[(triisopropylsilyl)oxy]phenyl}-1-ethyl-3-(3-hydroxy-2,2-dimethylpropyl)pyrrolo[3,2-b]pyridin-2-yl)-6-[(1 S)-1-methoxyethyl]pyridin-3-yl]piperazine-1-carboxylate (2.50 g, crude), which was used without further purification. LCMS (ESI) m/z: [M+H] calcd for C₆₃H₉₀N₈O₁₀Si: 1147.7; found 1147.5.

Step 9. To a solution of benzyl 4-[5-(5-{3-[(2S)-2-[(tert-butoxycarbonyl)amino]-3-[(3S)-3-(methoxycarbonyl)-1,2-diazinan-1-yl]-3-oxopropyl]-5-[(triisopropylsilyl)oxy]phenyl}-1-ethyl-3-(3-hydroxy-2,2-dimethylpropyl)pyrrolo[3,2-b]pyridin-2-yl)-6-[(1 S)-1-methoxyethyl]pyridin-3-yl]piperazine-1-carboxylate (2.50 g, crude) in H₂O (5 mL) and THE (5 mL) was added LiOH·H₂O (137 mg, 3.27 mmol) at 0° C. The reaction mixture was stirred for 2 h at 0° C. and was then acidified to pH 5 by the addition of 2 M aq. HCl. The aqueous mixture was extracted with EtOAc (3×20 mL) and the combined organic extracts were dried over Na₂SO₄, filtered, and concentrated under reduced pressure to give (3S)-1-[(2S)-3-{3-[2-(5-{4-[(benzyloxy)carbonyl]piperazin-1-yl}-2-[(1 S)-1-methoxyethyl]pyridin-3-yl)-1-ethyl-3-(3-hydroxy-2,2-dimethylpropyl)pyrrolo[3,2-b]pyridin-5-yl]-5-[(triisopropylsilyl)oxy]phenyl}-2-[(tert-butoxycarbonyl)amino]propanoyl]-1,2-diazinane-3-carboxylic acid (2.20 g, crude), which was used without further purification. LCMS (ESI) m/z: [M+H] calcd for C₆₂H₈₈N₈O₁₀Si: 1133.6; found 1133.6.

Step 10. To a solution of (3S)-1-[(2S)-3-{3-[2-(5-{4-[(benzyloxy)carbonyl]piperazin-1-yl}-2-[(1S)-1-methoxyethyl]pyridin-3-yl)-1-ethyl-3-(3-hydroxy-2,2-dimethylpropyl)pyrrolo[3,2-b]pyridin-5-yl]-5-[(triisopropylsilyl)oxy]phenyl}-2-[(tert-butoxycarbonyl)amino]propanoyl]-1,2-diazinane-3-carboxylic acid (2.20 g, crude) and DIPEA (5.02 g, 38.8 mmol) in DCM (60 mL) were added EDCI (4.52 g, 29.1 mmol) and HOBT (1.31 g, 9.71 mmol) at 0° C. The reaction mixture was stirred overnight at room temperature and was then quenched with H₂O (60 mL). The aqueous layer was extracted with DCM (3×20 mL) and the combined organic extracts were washed with brine (3×20 mL), dried over Na₂SO₄, filtered, and concentrated under reduced pressure. The resulting residue was purified by normal phase chromatography to give benzyl 4-(5-((6³S,4S)-4-((tert-butoxycarbonyl)amino)-1¹-ethyl-10,10-dimethyl-5,7-dioxo-2⁵-((triisopropylsilyl)oxy)-6¹,6²,6³,6⁴,6⁵,6⁶-hexahydro-1¹H-8-oxa-1(5,3)-pyrrolo[3,2-b]pyridina-6(1,3)-pyridazina-2(1,3)-benzenacycloundecaphane-1²-yl)-6-((S)-1-methoxyethyl)pyridin-3-yl)piperazine-1-carboxylate (1.00 g, 42% yield over 2 steps) as a yellow solid. LCMS (ESI) m/z: [M+H] calcd for C₆₂H₈₆N₈O₉Si: 1115.6 found 1115.5.

Step 11. To a solution of benzyl 4-(5-((6³S,4S)-4-((tert-butoxycarbonyl)amino)-1¹-ethyl-10,10-dimethyl-5,7-dioxo-2⁵-((triisopropylsilyl)oxy)-6¹,6²,6³,6⁴,6⁵,6⁶-hexahydro-1¹H-8-oxa-1(5,3)-pyrrolo[3,2-b]pyridina-6(1,3)-pyridazina-2(1,3)-benzenacycloundecaphane-1²-yl)-6-((S)-1-methoxyethyl)pyridin-3-yl)piperazine-1-carboxylate (1.00 g, 0.896 mmol) in i-PrOH (10 mL) was added 20% Pd(OH)₂/C (315 mg, 0.448 mmol) at room temperature under an atmosphere of N₂. The reaction mixture was stirred for 2 h at 40° C. under an atmosphere of H₂. The resulting mixture was then filtered, the filter cake was washed with EtOAc (3×20 mL), and the filtrated concentrated under reduced pressure to give tert-butyl ((6³S,4S)-11-ethyl-1²-(2-((S)-1-methoxyethyl)-5-(piperazin-1-yl)pyridin-3-yl)-10,10-dimethyl-5,7-dioxo-2⁵-((triisopropylsilyl)oxy)-6¹,6²,6³,6⁴,6⁵,6⁶-hexahydro-1¹H-8-oxa-1(5,3)-pyrrolo[3,2-b]pyridina-6(1,3)-pyridazina-2(1,3)-benzenacycloundecaphane-4-yl)carbamate (900 mg, crude), which was used without further purification. LCMS (ESI) m/z: [M+H] calcd for C₅₄H₈₀N₈O₇Si: 981.6; found 981.5.

Step 12. To a solution tert-butyl ((6³S,4S)-1¹-ethyl-1²-(2-((S)-1-methoxyethyl)-5-(piperazin-1-yl)pyridin-3-yl)-10,10-dimethyl-5,7-dioxo-2⁵-((triisopropylsilyl)oxy)-6¹,6²,6³,6⁴,6⁵,6⁶-hexahydro-1¹H-8-oxa-1(5,3)-pyrrolo[3,2-b]pyridina-6(1,3)-pyridazina-2(1,3)-benzenacycloundecaphane-4-yl)carbamate (900 mg, crude) and AcOH (165 mg, 2.75 mmol) in i-PrOH (10 mL) were added (1-ethoxycyclopropoxy)trimethylsilane (480 mg, 2.75 mmol) and NaBH₃CN (173 mg, 2.75 mmol) at room temperature. The reaction mixture was stirred for 2 h at 60° C. and was then quenched by the addition of H₂O (20 mL). The aqueous mixture was extracted with EtOAc (3×20 mL) and the combined organic extracts were dried over Na₂SO₄, filtered, and concentrated under reduced pressure to give tert-butyl ((6³S,4S)-1²-(5-(4-cyclopropylpiperazin-1-yl)-2-((S)-1-methoxyethyl) pyridin-3-yl)-1¹-ethyl-10,10-dimethyl-5,7-dioxo-2⁵-((triisopropylsilyl)oxy)-6¹,6²,6³,6⁴,6⁵,6⁶-hexahydro-1¹H-8-oxa-1(5,3)-pyrrolo[3,2-b]pyridina-6(1,3)-pyridazina-2(1,3)-benzenacycloundecaphane-4-yl)carbamate (800 mg, crude), which was used without further purification. LCMS (ESI) m/z: [M+H] calcd for C₅₇H₈₄N₈O₇Si: 1021.6; found 1021.4.

Step 13. To a solution of tert-butyl ((6³S,4S)-1²-(5-(4-cyclopropylpiperazin-1-yl)-2-((S)-1-methoxyethyl) pyridin-3-yl)-1¹-ethyl-10,10-dimethyl-5,7-dioxo-2⁵-((triisopropylsily)oxy)-6¹,6²,6³,6⁴,6⁵,6⁶-hexahydro-1¹H-8-oxa-1(5,3)-pyrrolo[3,2-b]pyridina-6(1,3)-pyridazina-2(1,3)-benzenacycloundecaphane-4-yl)carbamate (800 mg, crude) in DMF (10 mL) was added CsF (238 mg, 1.57 mmol) at room temperature. The reaction mixture was stirred for 2 h at room temperature and was then quenched by the addition of H₂O (20 mL). The aqueous mixture was extracted with EtOAc (3×20 mL), and the combined organic extracts were washed with brine (3×10 mL), dried over Na₂SO₄, filtered, and concentrated under reduced pressure. The resulting residue was purified by reversed phase chromatography to give tert-butyl ((6³S,4S)-1²-(5-(4-cyclopropylpiperazin-1-yl)-2-((S)-1-methoxyethyl)pyridin-3-yl)-1¹-ethyl-2⁵-hydroxy-10,10-dimethyl-5,7-dioxo-6¹,6²,6³,6⁴,6⁵,6⁶-hexahydro-1¹H-8-oxa-1(5,3)-pyrrolo[3,2-b]pyridina-6(1,3)-pyridazina-2(1,3)-benzenacycloundecaphane-4-yl)carbamate (M-atropisomer, 200 mg, 26% over 3 steps) as a yellow solid. LCMS (ESI) m/z: [M+H] calcd for C₄₈H₆₄N₈O₇: 865.5; found 865.5.

Step 14. To a solution of tert-butyl ((6³S,4S)-1²-(5-(4-cyclopropylpiperazin-1-yl)-2-((S)-1-methoxyethyl)pyridin-3-yl)-1¹-ethyl-2⁵-hydroxy-10,10-dimethyl-5,7-dioxo-6¹,6²,6³,6⁴,6⁵,6⁶-hexahydro-1¹H-8-oxa-1(5,3)-pyrrolo[3,2-b]pyridina-6(1,3)-pyridazina-2(1,3)-benzenacycloundecaphane-4-yl)carbamate (200 mg, 0.231 mmol) in DCM (2.0 mL) stirred at 0° C. was added TFA (2.0 mL). The resulting mixture was stirred for 2 h at room temperature, concentrated under reduced pressure, and the concentrate dissolved in EtOAc (50 mL). The organic solution was washed with sat. aq. NaHCO₃ (3×10 mL), dried over Na₂SO₄, filtered, and concentrated under reduced pressure to afford (6³S,4S)-4-amino-1²-(5-(4-cyclopropylpiperazin-1-yl)-2-((S)-1-methoxyethyl)pyridin-3-yl)-1¹-ethyl-2⁵-hydroxy-10,10-dimethyl-6¹,6²,6³,6⁴,6⁵,6⁶-hexahydro-1¹H-8-oxa-1(5,3)-pyrrolo[3,2-b]pyridina-6(1,3)-pyridazina-2(1,3)-benzenacycloundecaphane-5,7-dione (180 mg, crude) which was used without further purification. LCMS (ESI) m/z: [M+H] calcd for C₄₃H₅₆N₈O₅: 765.5; found 765.6.

Step 15. To a solution of (6³S,4S)-4-amino-1²-(5-(4-cyclopropylpiperazin-1-yl)-2-((S)-1-methoxyethyl)pyridin-3-yl)-1¹-ethyl-2⁵-hydroxy-10,10-dimethyl-6¹,6²,6³,6⁴,6⁵,6⁶-hexahydro-1 ¹H-8-oxa-1(5,3)-pyrrolo[3,2-b]pyridina-6(1,3)-pyridazina-2(1,3)-benzenacycloundecaphane-5,7-dione (180 mg, crude) and (2S)-2-{1-[(2S,3S)-1-(tert-butoxycarbonyl)-2-{[(4-methylbenzenesulfonyl)oxy]methyl}pyrrolidin-3-yl]-N-methylformamido}-3-methylbutanoic acid (180 mg, crude) in DMF (5.0 mL) stirred at 0° C. were added DIPEA (200 mL, 1.18 mmol) and COMU (202 mg, 0.470 mmol). The resulting mixture was stirred for 2 h at room temperature and was then quenched by the addition of H₂O (10 mL). The aqueous mixture was extracted with EtOAc (3×10 mL), and the combined organic extracts were washed with brine (3×7 mL), dried over Na₂SO₄, and concentrated under reduced pressure. Purification by normal phase chromatography afforded tert-butyl (2S,3S)-3-(((2S)-1-(((6³S,4S)-1²-(5-(4-cyclopropylpiperazin-1-yl)-2-((S)-1-methoxyethyl)pyridin-3-yl)-1¹-ethyl-2⁵-hydroxy-10,10-dimethyl-5,7-dioxo-6¹,6²,6³,6⁴,6⁵,6⁶-hexahydro-1¹H-8-oxa-1(5,3)-pyrrolo[3,2-b]pyridina-6(1,3)-pyridazina-2(1,3)-benzenacycloundecaphane-4-yl)amino)-3-methyl-1-oxobutan-2-yl)(methyl)carbamoyl)-2-((tosyloxy)methyl)pyrrolidine-1-carboxylate (170 mg, 58% yield over 2 steps). LCMS (ESI) m/z: [M+H] calcd for C₆₇H₉₀N₁₀O₁₂S: 1259.7; found 1259.6.

Step 16. To a solution of tert-butyl (2S,3S)-3-(((2S)-1-(((6³S,4S)-1²-(5-(4-cyclopropylpiperazin-1-yl)-2-((S)-1-methoxyethyl)pyridin-3-yl)-1¹-ethyl-2⁵-hydroxy-10,10-dimethyl-5,7-dioxo-6¹,6²,6³,6⁴,6⁵,6⁶-hexahydro-1¹H-8-oxa-1(5,3)-pyrrolo[3,2-b]pyridina-6(1,3)-pyridazina-2(1,3)-benzenacycloundecaphane-4-yl)amino)-3-methyl-1-oxobutan-2-yl)(methyl)carbamoyl)-2-((tosyloxy)methyl)pyrrolidine-1-carboxylate (170 mg, 0.135 mmol) and K₂CO₃ (186 mg, 1.35 mmol) in DMF (20 mL) stirred at room temperature was added KI (22.4 mg, 0.135 mmol). The resulting mixture was stirred overnight at 80° C. and was then quenched at room temperature by the addition of H₂O (20 mL). The aqueous mixture was extracted with EtOAc (3×20 mL), and the combined organic extracts were washed with brine (3×10 mL), dried over Na₂SO₄, filtered, and concentrated under reduced pressure to afford tert-butyl (3aS,6S,9S,15S,32aS)-21-(5-(4-cyclopropylpiperazin-1-yl)-2-((S)-1-methoxyethyl)pyridin-3-yl)-22-ethyl-6-isopropyl-5,19,19-trimethyl-4,7,10,16-tetraoxo-2,3,3a,4,5,6,7,8,9,10,13,14,15,16,18,19,20,22,32,32a-icosahydro-1H,12H-11,15-epimino-23,25-etheno-9,28-methano-26,30-(metheno)dipyrrolo[2,3-c:3′,4′-v][1,18]dioxa[6,9,12,24]tetraazacyclotriacontine-1-carboxylate (165 mg, crude) which was used without further purification. LCMS (ESI) m/z: [M+H] calcd for C₆₀H₈₂N₁₀O₉: 1087.6; found 1087.6.

Step 17. To a solution of tert-butyl (3aS,6S,9S,15S,32aS)-21-(5-(4-cyclopropylpiperazin-1-yl)-2-((S)-1-methoxyethyl)pyridin-3-yl)-22-ethyl-6-isopropyl-5,19,19-trimethyl-4,7,10,16-tetraoxo-2,3,3a,4,5,6,7,8,9,10,13,14,15,16,18,19,20,22,32,32a-icosahydro-1H,12H-11,15-epimino-23,25-etheno-9,28-methano-26,30-(metheno)dipyrrolo[2,3-c:3′,4′-v][1,18]dioxa[6,9,12,24]tetraazacyclotriacontine-1-carboxylate (165 mg, crude) in DCM (3.0 mL) stirred at 0° C. was added TFA (1.0 mL). The resulting mixture was stirred for 2 h at room temperature, concentrated under reduced pressure, and the concentrate dissolved in EtOAc (40 mL). The organic solution was washed with sat. aq. NaHCO₃ (3×10 mL), dried over Na₂SO₄, filtered, and concentrated under reduced pressure to afford (3aS,6S,9S,15S,32aS)-21-(5-(4-cyclopropylpiperazin-1-yl)-2-((S)-1-methoxyethyl)pyridin-3-yl)-22-ethyl-6-isopropyl-5,19,19-trimethyl-3,3a,5,6,8,9,14,15,18,19,20,22,32,32a-tetradecahydro-1H,12H-11,15-epimino-23,25-etheno-9,28-methano-26,30-(metheno)dipyrrolo[2,3-c:3′,4′-v][1,18]dioxa[6,9,12,24]tetraazacyclotriacontine-4,7,10,16(2H,13H)-tetraone (110 mg, crude) which was used without further purification. LCMS (ESI) m/z: [M+H] calcd for C₅₅H₇₄N₁₀O₇: 987.6; found 987.5.

Step 18. To a solution of (3aS,6S,9S,15S,32aS)-21-(5-(4-cyclopropylpiperazin-1-yl)-2-((S)-1-methoxyethyl)pyridin-3-yl)-22-ethyl-6-isopropyl-5,19,19-trimethyl-3,3a,5,6,8,9,14,15,18,19,20,22,32,32a-tetradecahydro-1 H,12H-11,15-epimino-23,25-etheno-9,28-methano-26,30-(metheno)dipyrrolo[2,3-c:3′,4′-v][1,18]dioxa[6,9,12,24]tetraazacyclotriacontine-4,7,10,16(2H,13H)-tetraone (110 mg, crude) and 2-fluoro-2-methylpropanoic acid (23.6 mg, 0.222 mmol) in DCM (5.0 mL) stirred at 0° C. were added DIPEA (390 mL, 2.22 mmol) and COMU (120 mg, 0.278 mmol). The resulting mixture was stirred for 2 h at room temperature then quenched by the addition of H₂O (10 mL). The aqueous mixture was extracted with DCM (3×10 mL), and the combined organic extracts were dried with Na₂SO₄, filtered, and concentrated under reduced pressure. Purification by reversed phase chromatography afforded (3aS,6S,9S,15S,32aS)-21-(5-(4-cyclopropylpiperazin-1-yl)-2-((S)-1-methoxyethyl)pyridin-3-yl)-22-ethyl-1-(2-fluoro-2-methylpropanoyl)-6-isopropyl-5,19,19-trimethyl-3,3a,5,6,8,9,14,15,18,19,20,22,32,32a-tetradecahydro-1H,12H-11,15-epimino-23,25-etheno-9,28-methano-26,30-(metheno)dipyrrolo[2,3-c:3′,4′-v][1,18]dioxa[6,9,12,24]tetraazacyclotriacontine-4,7,10,16(2H,13H)-tetraone (35.4 mg, 24% yield over 3 steps). LCMS (ESI) m/z: [M+H] calcd for C₅₉H₇₉FN₁₀O₈: 1075.6; found 1075.4; ¹H NMR (400 MHz, DMSO-d₆) δ 8.48 (d, J=2.8 Hz, 1H), 8.37 (s, 1H), 8.04-7.90 (m, 3H), 7.60 (s, 1H), 7.35 (d, J=2.9 Hz, 1H), 6.99 (s, 1H), 5.44-5.24 (m, 2H), 4.72-4.42 (m, 3H), 4.32 (t, J=9.8 Hz, 2H), 4.10 (p, J=7.4 Hz, 3H), 4.00-3.67 (m, 3H), 3.60-3.47 (m, 1H), 3.30-3.07 (m, 6H), 3.07-2.75 (m, 5H), 2.74-2.60 (m, 5H), 2.17 (d, J=13.2 Hz, 2H), 2.15 (s, 3H), 2.01-1.87 (m, 2H), 1.84-1.77 (m, 2H), 1.73-1.51 (m, 10H), 1.41 (d, J=6.2 Hz, 3H), 1.30-1.08 (m, 4H), 0.77 (d, J=6.4 Hz, 3H), 0.69-0.56 (m, 9H), 0.51-0.29 (m, 4H).

### Example A334. Synthesis of (2²,2^{3a},2⁵S,2⁸S,2^{10a}S,2^{13a}R,2¹⁴,4³S)-1²-(5-(4-cyclopropylpiperazin-1-yl)-2-((S)-1-methoxyethyl)pyridin-3-yl)-1¹-ethyl-2⁸-isopropyl-2⁹,8,8-trimethyl-2¹³-propionyl-2⁴,2⁵,2⁶,2⁷,2⁸,2⁹,2¹⁰,2^{10a},2¹¹,2¹²,2¹³,2^{13a},4¹,4²,4³,4⁴,4⁵,4⁶-octadecahydro-1¹H-6-oxa- 2(2,5)-pyrrolo[3,2-f]thiazolo[5,4-j][1,4]diazacyclotridecina-1(5,3)-indola-4(1,3)-pyridazinacyclononaphane-2⁷,2¹⁰,3,5-tetraone

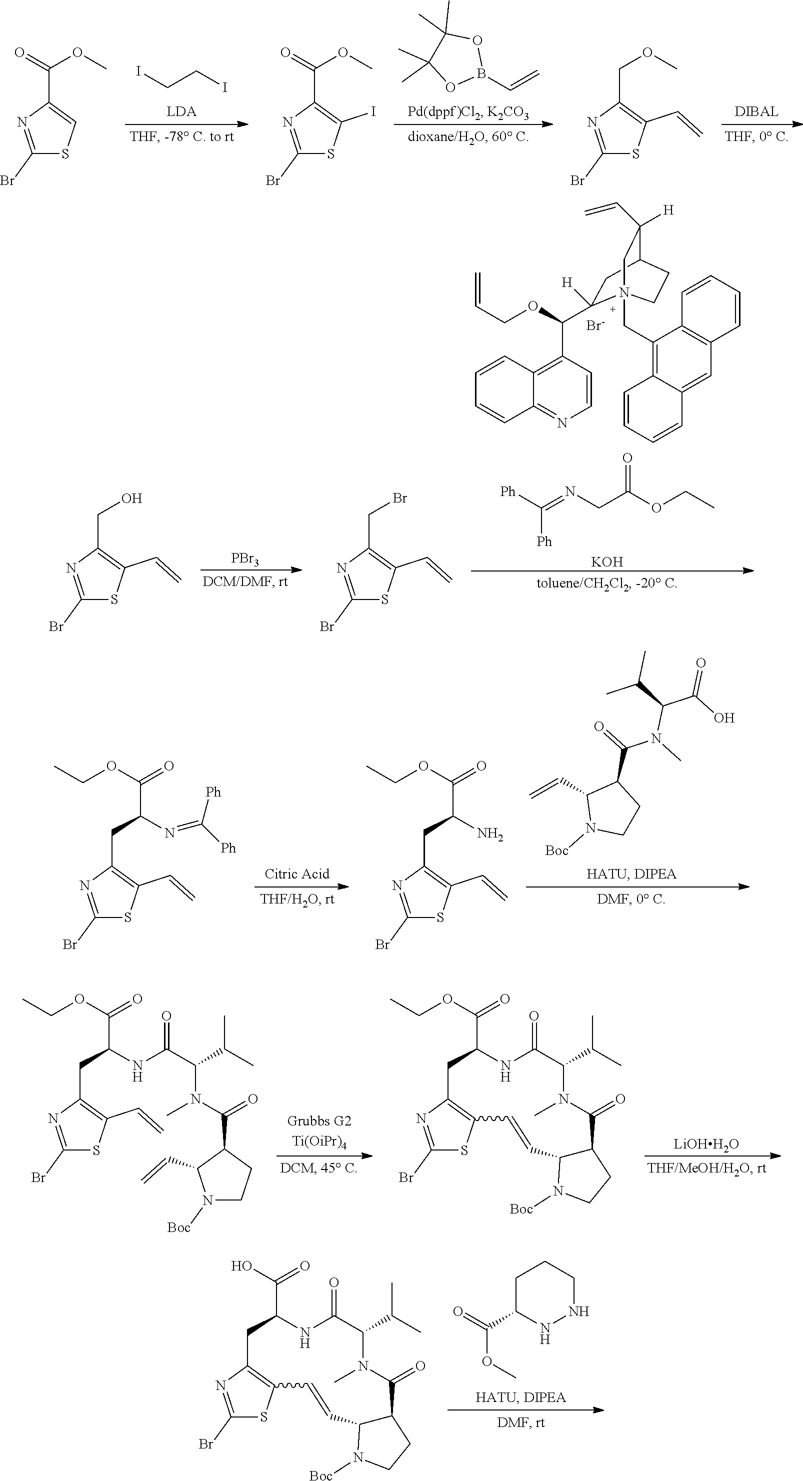

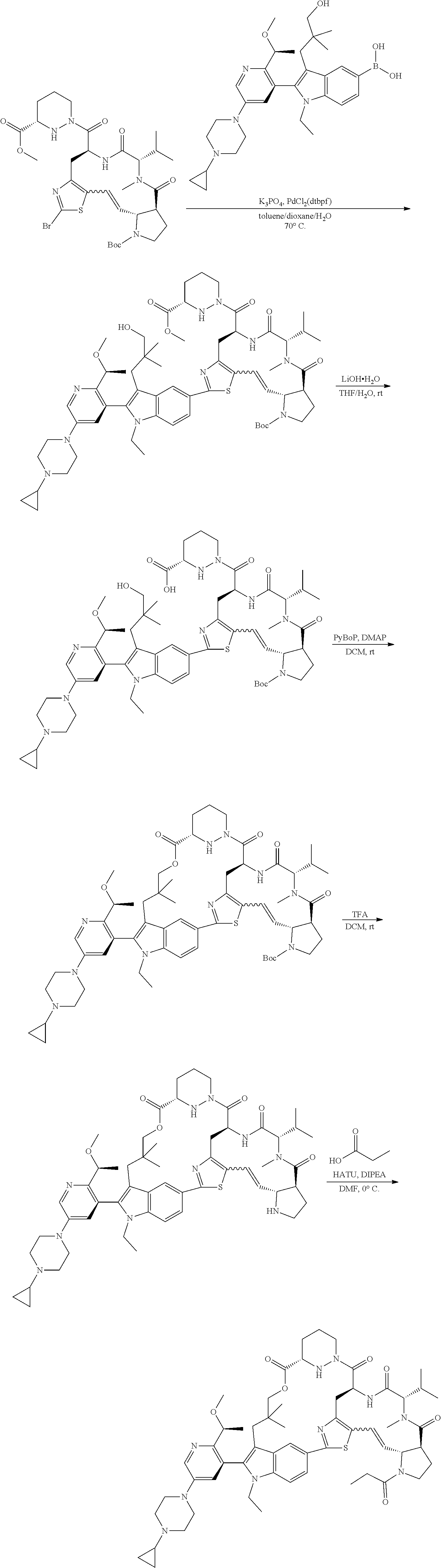

Step 1. To a solution of LDA (50 mL, 2 M in THF) in THF (200 mL) stirred at −78° C. was added methyl 2-bromothiazole-4-carboxylate (11 g, 49.5 mmol). The resulting mixture was stirred for 30 min at −78° C. followed by the addition over 30 min of 1,2-diiodoethane (41.9 g, 149 mmol). The reaction mixture was stirred for an additional 2 h at −78° C., stirred at room temperature for 12 h, then quenched at −30° C. by the addition of sat. aq. NH₄Cl (50 mL). The aqueous mixture was extracted with EtOAc (3×100 mL), and the combined organic extracts were dried over Na₂SO₄, filtered, and concentrated under reduced pressure. Purification by normal phase chromatography afforded methyl 2-bromo-5-iodothiazole-4-carboxylate (6.70 g, 39% yield) as a yellow solid. LCMS (ESI) m/z: [M+H] calcd for C₅H₃BrINO₂S: 347.8; found 347.7.

Step 2. To a solution of methyl 2-bromo-5-iodothiazole-4-carboxylate (6.66 g, 19.1 mmol), K₂CO₃ (7.94 g, 57.4 mmol) and Pd(dppf)Cl₂ (1.40 g, 1.91 mmol) in dioxane (75 mL) and H₂O (15 mL) was added a solution of 4,4,5,5-tetramethyl-2-vinyl-1,3,2-dioxaborolane (2.95 g, 19.1 mmol) in dioxane (75 mL). The resulting mixture was stirred for 2 h at 60° C. and was then quenched at 0° C. by the addition of H₂O (50 mL). The aqueous mixture was extracted with EtOAc (3×30 mL), and the combined organic extracts were dried over Na₂SO₄, filtered, and concentrated under reduced pressure. Purification by normal phase chromatography afforded methyl 2-bromo-5-vinylthiazole-4-carboxylate (3.20 g, 67% yield) as a brown solid. LCMS (ESI) m/z: [M+H] calcd for C₇H₆BrNO₂S: 247.9; found 247.9.

Step 3. To a solution of methyl 2-bromo-5-vinylthiazole-4-carboxylate (3.66 g, 14.8 mmol) in THE (40 m) at 0° C. was added DIBAL (5.25 g, 36.883 mmol). The resulting mixture was stirred for 2 h at 0° C. and was then quenched by the addition of MeOH (50 mL) and sat. aq. potassium sodium tartrate (50 mL). The aqueous mixture was extracted with EtOAc (3×50 mL), and the combined organic extracts were dried over Na₂SO₄, filtered, and concentrated under reduced pressure. Purification by normal phase chromatography afforded (2-bromo-5-vinylthiazol-4-yl)methanol (2.12 g, 65% yield) as a light yellow oil. LCMS (ESI) m/z: [M+H] calcd for C₆H₆BrNOS: 220.0; found 219.9.

Step 4. To a solution of (2-bromo-5-vinylthiazol-4-yl)methanol (2.12 g, 9.63 mmol) in DCM (20 mL) and DMF (4.0 mL) at 0° C. was added PBr₃ (3.91 g, 14.4 mmol). The resulting mixture was stirred for 10 h at room temperature and was then quenched at 0° C. by addition of H₂O. The aqueous mixture was extracted with DCM (3×15 mL), and the combined organic extracts were dried over Na₂SO₄, filtered, and concentrated under reduced pressure. Purification by normal phase chromatography afforded 2-bromo-4-(bromomethyl)-5-vinylthiazole (2.59 g, 95% yield) as a white solid. LCMS (ESI) m/z: [M+H] calcd for C₆H₅Br₂NS: 281.9; found 281.8.

Step 5. To a solution of ethyl 2-[(diphenylmethylidene)amino]acetate (2.35 g, 8.80 mmol) in toluene (21 mL) and DCM (9 mL) stirred at −20° C. were added O-Allyl-N-(9-anthracenylmethyl)cinchonidinium bromide (749 mg, 1.24 mmol) and 9M aq. KOH (30 mL). To the resulting mixture was added over 10 min 2-bromo-4-(bromomethyl)-5-vinylthiazole (2.49 g, 8.799 mmol). The reaction mixture was stirred overnight at −20° C. then quenched at −30° C. by the addition of H₂O (30 mL). The aqueous mixture was extracted with EtOAc (3×20 mL), and the combined organic extracts were dried over Na₂SO₄, filtered, and concentrated under reduced pressure to afford ethyl (S)-3-(2-bromo-5-vinylthiazol-4-yl)-2-((diphenylmethylene)amino)propanoate (5.6 g, crude) which was used in the next reaction without further purification. LCMS (ESI) m/z: [M+H] calcd for C₂₃H₃₁BrN₂O₂S: 469.1; found 469.0.

Step 6. To a solution of ethyl (S)-3-(2-bromo-5-vinylthiazol-4-yl)-2-((diphenylmethylene)amino)propanoate (5.16 g, 10.9 mmol) in THE (30 mL) and H₂O (30 mL) was added citric acid (10.6 g, crude). The resulting mixture was stirred for 5 h at room temperature then quenched at 0° C. by the addition of H₂O (20 mL). The aqueous mixture was extracted with MTBE (3×20 mL) and the combined organic extracts were discarded. The aqueous phase was then basified to pH=8 with sat. aq. NaHCO₃ and extracted with DCM (3×20 mL) and the combined organic extracts were dried over Na₂SO₄, filtered, and concentrated under reduced pressure. Purification by normal phase chromatography afforded ethyl (S)-2-amino-3-(2-bromo-5-vinylthiazol-4-yl)propanoate (2.25 g, 91% yield over 2 steps) as a colorless oil. LCMS (ESI) m/z: [M+H] calcd for C₁₀H₁₃BrN₂O₂S: 305.0; found 304.9.

Step 7. To a solution of ethyl (S)-2-amino-3-(2-bromo-5-vinylthiazol-4-yl)propanoate (1.03 g, 3.37 mmol) and DIPEA (17.4 g, 135 mmol) in DMF (10 mL) stirred at 0° C. were added N-((2R,3S)-1-(tert-butoxycarbonyl)-2-vinylpyrrolidine-3-carbonyl)-N-methyl-L-valine (1.56 g, 4.39 mmol) and HATU (1.92 g, 5.06 mmol). The resulting mixture was stirred for 2 h at 0° C. and was then quenched by the addition of H₂O (10 mL). The aqueous mixture was extracted with EtOAc (3×15 mL), and the combined organic extracts were washed with brine (3×50 mL), dried over Na₂SO₄, filtered, and concentrated under reduced pressure. Purification by normal phase chromatography afforded tert-butyl (2R,3S)-3-(((S)-1-(((S)-3-(2-bromo-5-vinylthiazol-4-yl)-1-ethoxy-1-oxopropan-2-yl)amino)-3-methyl-1-oxobutan-2-yl)(methyl)carbamoyl)-2-vinylpyrrolidine-1-carboxylate (635 mg, 29% yield). This material was used in the subsequent reactions without further purification. LCMS (ESI) m/z: [M+H−100] calcd for C₂₈H₄₁BrN₄O₆S: 541.2; found 541.1.

Step 8. To a solution of tert-butyl (2R,3S)-3-(((S)-1-(((S)-3-(2-bromo-5-vinylthiazol-4-yl)-1-ethoxy-1-oxopropan-2-yl)amino)-3-methyl-1-oxobutan-2-yl)(methyl)carbamoyl)-2-vinylpyrrolidine-1-carboxylate (635 mg, 0.990 mmol) and Grubbs G2 catalyst (505 mg, 0.595 mmol) in DCM (35 mL) stirred at room temperature was added Ti(OiPr)₄ (169 mg, 0.594 mmol). The resulting mixture was stirred overnight at 45° C. and was then quenched at 0° C. by the addition of H₂O (30 mL). The aqueous mixture was extracted with DCM (3×30 mL), and the combined organic extracts were dried over Na₂SO₄, filtered, and concentrated under reduced pressure. Purification by reversed phase chromatography 13-(tert-butyl) 5-ethyl (5S,8S,10aS,13aR)-2-bromo-8-isopropyl-9-methyl-7,10-dioxo-5,6,7,8,9,10,10a,11,12,13a-decahydropyrrolo[3,2-f]thiazolo[5,4-][1,4]diazacyclotridecine-5,13(4F)-dicarboxylate (121 mg, 20% yield, mixture of the E- and Z-isomers) as a light brown oil. LCMS (ESI) m/z: [M+H] calcd for C₂₆H₃₇BrN₄O₆S: 613.2; found 613.0.

Step 9. To a solution of 13-(tert-butyl) 5-ethyl (5S,8S,10aS,13aR)-2-bromo-8-isopropyl-9-methyl-7,10-dioxo-5,6,7,8,9,10,10a,11,12,13a-decahydropyrrolo[3,2-f]thiazolo[5,4-j][1,4]diazacyclotridecine-5,13(4F)-dicarboxylate (124 mg, 0.218 mmol) in MeOH (1.0 mL), THE (1.0 mL) and H₂O (1.0 mL) was added LiOH·H₂O (9.7 mg, 0.404 mmol). The resulting mixture was stirred for 2 h at room temperature, concentrated under reduced pressure, and the concentrate acidified to pH=5 with 1 M aq. HCl. The aqueous mixture was extracted with DCM (3×20 mL), and the combined organic extracts were dried over Na₂SO₄, filtered, and concentrated under reduced pressure to afford (5S,8S,10aS,13aR,E)-2-bromo-13-(tert-butoxycarbonyl)-8-isopropyl-9-methyl-7,10-dioxo-4,5,6,7,8,9,10,10a,11,12,13,13a-dodecahydropyrrolo[3,2-f]thiazolo[5,4-j][1,4]diazacyclotridecine-5-carboxylic acid (142 mg, crude) which was taken directly to the next reaction without further purification. LCMS (ESI) m/z: [M+H] calcd for C₂₄H₃₃BrN₄O₆S: 585.1; found 585.3.

Step 10. To a solution of methyl (S)-hexahydropyridazine-3-carboxylate (69.9 mg, 0.486 mmol) and DIPEA (313 mg, 2.43 mmol) in DMF (1.0 mL) at 0° C. were added (5S,8S,10aS,13aR)-2-bromo-13-(tert-butoxycarbonyl)-8-isopropyl-9-methyl-7,10-dioxo-4,5,6,7,8,9,10,10a,11,12,13,13a-dodecahydropyrrolo[3,2-f]thiazolo[5,4-j][1,4]diazacyclotridecine-5-carboxylic acid (142 mg, crude) and HATU (184 mg, 0.486 mmol). The resulting mixture was stirred for 1 h at room temperature and was then quenched at 0° C. by the addition of H₂O (10 mL). The aqueous mixture was extracted with EtOAc (3×10 mL, and the combined organic extracts were washed with brine (3×30 mL), dried over Na₂SO₄, filtered, and concentrated under reduced pressure. Purification by reversed phase chromatography afforded tert-butyl (5S,8S,10aS,13aR)-2-bromo-8-isopropyl-5-((S)-3-(methoxycarbonyl)hexahydropyridazine-1-carbonyl)-9-methyl-7,10-dioxo-5,6,7,8,9,10,10a,11,12,13a-decahydropyrrolo[3,2-f]thiazolo[5,4-j][1,4]diazacyclotridecine-13(4H)-carboxylate (154 mg, 99% yield over 2 steps) as a light brown oil. LCMS (ESI) m/z: [M+H] calcd for C₃₀H₄₃BrN₆O₇S: 711.2; found 711.1.

Step 11. To a solution tert-butyl (5S,8S,10aS,13aR)-2-bromo-8-isopropyl-5-((S)-3-(methoxycarbonyl)hexahydropyridazine-1-carbonyl)-9-methyl-7,10-dioxo-5,6,7,8,9,10,10a,11,12,13a-decahydropyrrolo[3,2-f]thiazolo[5,4-][1,4]diazacyclotridecine-13(4H)-carboxylate (150 mg, 0.211 mmol) and (S)-(2-(5-(4-cyclopropylpiperazin-1-yl)-2-(1-methoxyethyl)pyridin-3-yl)-1-ethyl-3-(3-hydroxy-2,2-dimethylpropyl)-1H-indol-5-yl)boronic acid (169 mg, 0.317 mmol) in toluene (1.5 mL), dioxane (500 mL) and H₂O (500 mL) stirred at room temperature were added K₃PO₄ (89.5 mg, 0.422 mmol) and PdCl₂(dtbpf) (13.7 mg, 0.021 mmol). The resulting mixture was stirred for 3 h at 70° C. and was then quenched at 0° C. by the addition of H₂O (10 mL). The aqueous mixture was extracted with EtOAc (3×20 mL), and the combined organic extracts were dried over Na₂SO₄, filtered, and concentrated under reduced pressure. Purification by normal phase chromatography afforded tert-butyl (5S,8S,10aS,13aR)-2-(2-(5-(4-cyclopropylpiperazin-1-yl)-2-((S)-1-methoxyethyl)pyridin-3-yl)-1-ethyl-3-(3-hydroxy-2,2-dimethylpropyl)-1H-indol-5-yl)-8-isopropyl-5-((S)-3-(methoxycarbonyl)hexahydropyridazine-1-carbonyl)-9-methyl-7,10-dioxo-5,6,7,8,9,10,10a,11,12,13a-decahydropyrrolo[3,2-f]thiazolo[5,4-j][1,4]diazacyclotridecine-13(4H)-carboxylate (137 mg, 58% yield) as a light brown solid. LCMS (ESI) m/z: [M+H] calcd for C₆₀H₈₄N₁₀O₉S: 1121.6; found 1121.6.

Step 12. To a solution of tert-butyl (5S,8S,10aS,13aR)-2-(2-(5-(4-cyclopropylpiperazin-1-yl)-2-((S)-1-methoxyethyl)pyridin-3-yl)-1-ethyl-3-(3-hydroxy-2,2-dimethylpropyl)-1H-indol-5-yl)-8-isopropyl-5-((S)-3-(methoxycarbonyl)hexahydropyridazine-1-carbonyl)-9-methyl-7,10-dioxo-5,6,7,8,9,10,10a,11,12,13a-decahydropyrrolo[3,2-f]thiazolo[5,4-j][1,4]diazacyclotridecine-13(4H)-carboxylate (135 mg, 0.120 mmol) in THE (1.0 mL) and H₂O (1.0 mL) was added LiOH·H₂O (10.1 mg, 0.240 mmol). The resulting mixture was stirred for 2 h at room temperature, concentrated under reduced pressure, and the concentrate acidified to pH=5 with 1M aq. HCl. The aqueous mixture was extracted with DCM (3×15 mL) and the combined organic extracts were dried over Na₂SO₄, filtered, and concentrated under reduced pressure to afford (S)-1-((5S,8S,10aS,13aR)-13-(tert-butoxycarbonyl)-2-(2-(5-(4-cyclopropylpiperazin-1-yl)-2-((S)-1-methoxyethyl)pyridin-3-yl)-1-ethyl-3-(3-hydroxy-2,2-dimethylpropyl)-1H-indol-5-yl)-8-isopropyl-9-methyl-7,10-dioxo-4,5,6,7,8,9,10,10a,11,12,13,13a-dodecahydropyrrolo[3,2-f]thiazolo[5,4-j][1,4]diazacyclotridecine-5-carbonyl)hexahydropyridazine-3-carboxylic acid (184 mg, crude) which was used in the next reaction without further purification. LCMS (ESI) m/z: [M+H] calcd for C₅₉H₈₂N₁₀O₉S: 1107.6; found 1107.6.

Step 13. To a solution of (S)-1-((5S,8S,10aS,13aR)-13-(tert-butoxycarbonyl)-2-(2-(5-(4-cyclopropylpiperazin-1-yl)-2-((S)-1-methoxyethyl)pyridin-3-yl)-1-ethyl-3-(3-hydroxy-2,2-dimethylpropyl)-1H-indol-5-yl)-8-isopropyl-9-methyl-7,10-dioxo-4,5,6,7,8,9,10,10a,11,12,13,13a-dodecahydropyrrolo[3,2-f]thiazolo[5,4-j][1,4]diazacyclotridecine-5-carbonyl)hexahydropyridazine-3-carboxylic acid (115 mg, crude) and DMAP (50.8 mg, 0.416 mmol) in DCM (11 mL) stirred at 0° C. was added PyBOP (540 mg, 1.04 mmol). The resulting mixture was stirred for 3 h at room temperature and was then quenched at 0° C. by the addition of H₂O (10 mL). The aqueous mixture was extracted with DCM (3×15 mL) and the combined organic extracts were dried over Na₂SO₄, filtered, and concentrated under reduced pressure. Purification by reversed phase chromatography afforded tert-butyl (2²,2^{3a},2⁵S,2⁸S,2^{10a}S,2^{13a}R,2¹⁴,4³S)-1²-(5-(4-cyclopropylpiperazin-1-yl)-2-((S)-1-methoxyethyl)pyridin-3-yl)-1¹-ethyl-2⁸-isopropyl-2⁹,8,8-trimethyl-2⁷,2¹⁰,3,5-tetraoxo-2⁴,2⁵,2⁶,2⁷,2⁸,2⁹,2¹⁰,2^{10a},2¹¹,2¹²,2¹³,2^{13a},4¹,4²,4³,4⁴,4⁵,4⁶- octadecahydro-1¹H-6-oxa-2(2,5)-pyrrolo[3,2-f]thiazolo[5,4-j][1,4]diazacyclotridecina-1(5,3)-indola-4(1,3)-pyridazinacyclononaphane-2¹³-carboxylate (34 mg, 42% yield over 2 steps) as a light brown oil. LCMS (ESI) m/z: [M+H] calcd for C₅₉H₈₀N₁₀O₈S: 1089.6; found 1089.6.

Step 14. To a solution of tert-butyl (2²,2^{3a},2⁵S,2⁸S,2^{10a}S,2^{13a}R,2¹⁴,4³S)-1²-(5-(4-cyclopropylpiperazin-1-yl)-2-((S)-1-methoxyethyl)pyridin-3-yl)-1¹-ethyl-2⁸-isopropyl-2⁹,8,8-trimethyl-2⁷ 2¹⁰,3,5-tetraoxo-2⁴,2⁵,2⁶,2⁷,2⁸,2⁹,2¹⁰,2^{10a},2¹¹,2¹²,2¹³,2^{13a},4¹,4²,4³,4⁴,4⁵,4⁶- octadecahydro-1¹H-6-oxa-2(2,5)-pyrrolo[3,2-f]thiazolo[5,4-j][1,4]diazacyclotridecina-1(5,3)-indola-4(1,3)-pyridazinacyclononaphane-2¹³-carboxylate (32 mg, 0.029 mmol) in DCM (1.0 mL) was added TFA (200 mL). The resulting mixture was stirred for 2 h at room temperature, diluted with DCM (10 mL), and concentrated under reduced pressure to afford (2²Z,2^{3a}Z,2⁵S,2⁸S,2^{10a}S,2¹³aR,2¹⁴,4³S)-1²-(5-(4-cyclopropylpiperazin-1-yl)-2-((S)-1-methoxyethyl)pyridin-3-yl)-1¹-ethyl-2⁸-isopropyl-2⁹,8,8-trimethyl-2⁴,2⁵,2⁶,2⁷,2⁸,2⁹,2¹⁰,2^{10a},2¹¹,2¹²,2¹³,2^{13a},4¹,4²,4³,4⁴,4⁵,4⁶-octadecahydro-1¹H-6-oxa- 2(2,5)-pyrrolo[3,2-f]thiazolo[5,4-j][1,4]diazacyclotridecina-1(5,3)-indola-4(1,3)-pyridazinacyclononaphane-2⁷,2¹⁰,3,5-tetraone (29 mg, crude) which was used in the next reaction without further purification. LCMS (ESI) m/z: [M+H] calcd for C₅₄H₇₂N₁₀O₆S: 989.6; found 989.4.

Step 15. To a solution of (2²,2^{3a},2⁵S,2⁸S,2^{10a}S,2^{13a}R,2¹⁴,4³S)-1²-(5-(4-cyclopropylpiperazin-1-yl)-2-((S)-1-methoxyethyl)pyridin-3-yl)-1¹-ethyl-2⁸-isopropyl-2⁹,8,8-trimethyl-2⁴,2⁵,2⁶,2⁷,2⁸,2⁹,2¹⁰,2^{10a},2¹¹,2¹²,2¹³,2^{13a},4¹,4²,4³,4⁴,4⁵,4⁶-octadecahydro-1¹H-6-oxa- 2(2,5)-pyrrolo[3,2-f]thiazolo[5,4-j][1,4]diazacyclotridecina-1(5,3)-indola-4(1,3)-pyridazinacyclononaphane-2⁷,2¹⁰,3,5-tetraone (27 mg, crude) and DIPEA (70.5 mg, 0.540 mmol) in DMF (500 mL) stirred at 0° C. were added propionic acid (4.0 mg, 0.054 mmol) and HATU (15.6 mg, 0.041 mmol). The resulting mixture was stirred for 1 h at 0° C. and was then quenched by the addition of H₂O (2 mL). The aqueous mixture was extracted with EtOAc (3×5 mL), and the combined organic extracts were washed with brine (3×20 mL), dried over Na₂SO₄, filtered, and concentrated under reduced pressure. Purification by reversed phase chromatography yielded (2²,2^{3a},2⁵S,2⁸S,2^{10a}S,2^{13a}R,2¹⁴,4³S)-1²-(5-(4-cyclopropylpiperazin-1-yl)-2-((S)-1-methoxyethyl)pyridin-3-yl)-1¹-ethyl-2⁸-isopropyl-2⁹,8,8-trimethyl-2¹³-propionyl-2⁴,2⁵,2⁶,2⁷,2⁸,2⁹,2¹⁰,2^{10a},2¹¹,2¹²,2¹³,2^{13a},4¹,4²,4³,4⁴,4⁵,4⁶-octadecahydro-1¹H-6-oxa- 2(2,5)-pyrrolo[3,2-f]thiazolo[5,4-j][1,4]diazacyclotridecina-1(5,3)-indola-4(1,3)-pyridazinacyclononaphane-2⁷,2¹⁰,3,5-tetraone (1.6 mg, 5.6% yield over 2 steps) as a white solid. LCMS (ESI) m/z: [M+H] calcd for C₅₇H₇₆N₁₀O₇S: 1045.6; found 1045.6; ¹H NMR (300 MHz, DMSO-d₆) δ 8.55-8.37 (m, 2H), 7.74-7.45 (m, 2H), 7.30-6.82 (m, 2H), 5.89 (m, 1H), 5.64 (t, J=9.4 Hz, 1H), 5.08 (d, J=8.2 Hz, 1H), 4.79-4.70 (m, 1H), 4.50-4.04 (m, 6H), 3.87-3.81 (m, 1H), 3.67-3.46 (m, 4H), 3.24 (s, 8H), 3.00-2.98 (m, 2H), 2.86-2.60 (m, 8H), 2.47-1.87 (m, 7H), 1.85-1.44 (m, 4H), 1.34 (d, J=6.0 Hz, 3H), 1.24 (s, 2H), 1.15-0.92 (m, 6H), 0.92-0.85 (m, 4H), 0.85-0.69 (m, 6H), 0.47-0.25 (m, 6H).

### Example A340. Synthesis of (3aS,6S,9S,15S,32aS)-21-(5-(4-cyclopropylpiperazin-1-yl)-2-((S)-1-methoxyethyl)pyridin-3-yl)-22-ethyl-19,19-difluoro-1-(2-fluoro-2-methylpropanoyl)-6-isopropyl-5-methyl-3,3a,5,6,8,9,14,15,18,19,20,22,32,32a-tetradecahydro-1H,12H-11,15-epimino-23,25-etheno-9,28-methano-26,30-(metheno)dipyrrolo[2,3-c:3′,4′-v][1,18]dioxa[6,9,12]triazacyclotriacontine-4,7,10,16(2H,13H)-tetraone

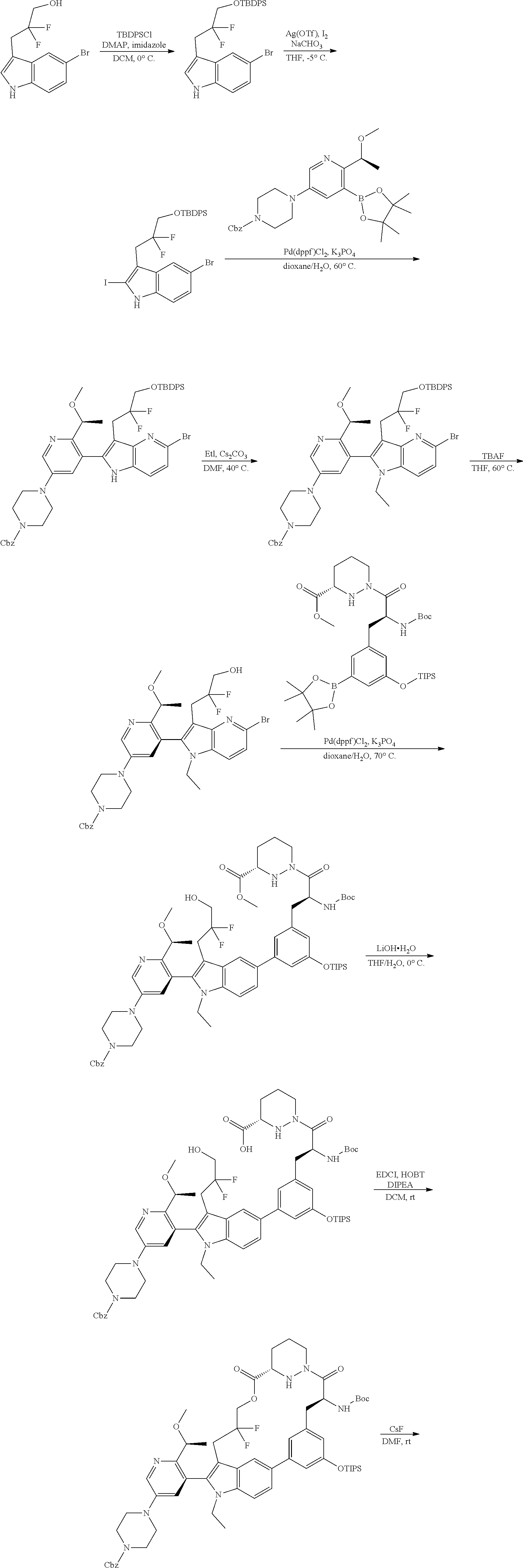

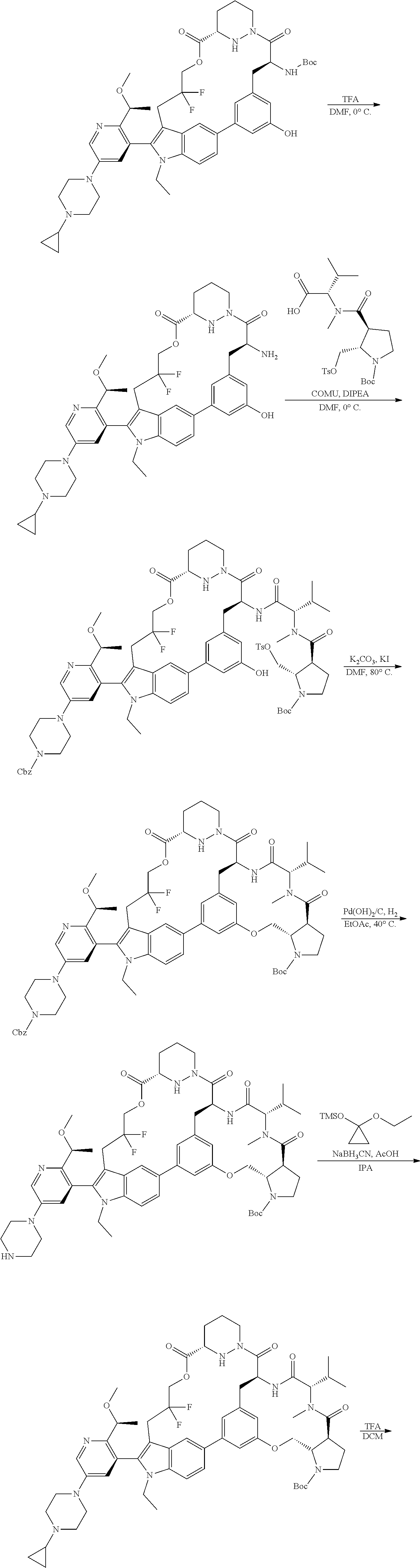

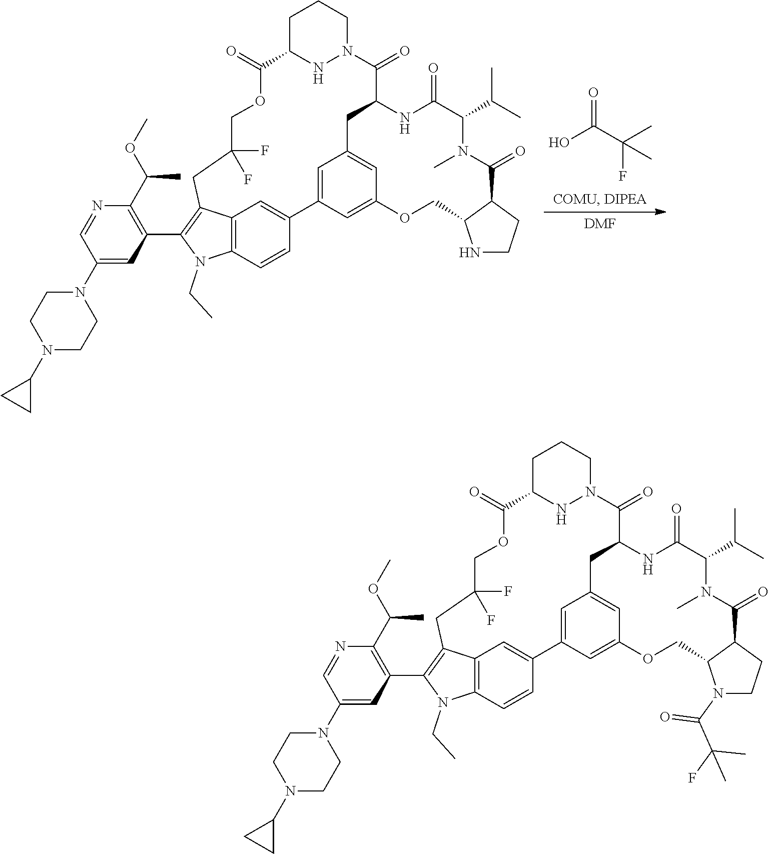

Step 1. To a solution of 3-(5-bromo-1H-indol-3-yl)-2,2-difluoropropan-1-ol (10 g, 34.5 mmol) and imidazole (5.87 g, 86.2 mmol) in DMF (100 mL) at 0° C. were added DMAP (0.84 g, 6.894 mmol) and TBDPSCI (28.4 g, 103 mmol). The resulting mixture was stirred overnight at 0° C. then diluted with brine (300 mL). The aqueous mixture was extracted with EtOAc (2×100 mL), and the combined organic extracts were dried over Na₂SO₄, filtered, and concentrated under reduced pressure to afford 5-bromo-3-(3-((tert-butyldiphenylsilyl)oxy)-2,2-difluoropropyl)-1H-indole (23.0 g, crude), which was used without further purification. LCMS (ESI) m/z: [M+H] calcd for C₂₇H₂₈BrF₂NOSi: 528.1; found 528.2.

Step 2. To a solution of 5-bromo-3-(3-((tert-butyldiphenylsilyl)oxy)-2,2-difluoropropyl)-1H-indole (23.0 g, crude) and NaHCO₃ (7.31 g, 87.0 mmol) in THE (230 mL) at −5° C. were added silver triflate (13.4 g, 52.2 mmol) and 12 (11.1 g, 43.5 mmol). The resulting mixture was stirred for 3 h at −5° C. then quenched at 0° C. by the addition of H₂O (100 mL). The aqueous mixture was extracted with EtOAc (3×150 mL), and the combined organic extracts were concentrated under reduced pressure to afford 5-bromo-3-(3-((tert-butyldiphenylsilyl)oxy)-2,2-difluoropropyl)-2-iodo-1H-indole (23.0 g, crude), which was used without further purification. LCMS (ESI) m/z: [M+H] calcd for C₂₇H₂₇BrF₂INOSi: 654.0; found 653.9.

Step 3. To a solution of 5-bromo-3-(3-((tert-butyldiphenylsilyl)oxy)-2,2-difluoropropyl)-2-iodo-1H-indole (17.0 g, crude), benzyl (S)-4-(6-(1-methoxyethyl)-5-(4,4,5,5-tetramethyl-1,3,2-dioxaborolan-2-yl)pyridin-3-yl)piperazine-1-carboxylate (11.4 g, 28.6 mmol) and K₂CO₃ (10.8 g, 77.9 mmol) in dioxane (150 mL) and H₂O (30 mL) at room temperature was added Pd(dppf)Cl₂-DCM (2.12 g, 2.59 mmol). The resulting mixture was stirred for 3 h at 60° C. then diluted with brine (100 mL). The aqueous mixture was extracted with EtOAc (2×100 mL), and the combined organic extracts were concentrated under reduced pressure. Purification by normal phase chromatography afforded benzyl (S)-4-(5-(5-bromo-3-(3-((tert-butyldiphenylsilyl)oxy)-2,2-difluoropropyl)-1H-indol-2-yl)-6-(1-methoxyethyl)pyridin-3-yl)piperazine-1-carboxylate (10.0 g, 44% yield over 3 steps) as a brown solid. LCMS (ESI) m/z: [M+H] calcd for C₄₇H₅₁BrF₂N₄O₄Si: 881.3; found 881.1.

Step 4. To a solution of benzyl (S)-4-(5-(5-bromo-3-(3-((tert-butyldiphenylsilyl)oxy)-2,2-difluoropropyl)-1H-indol-2-yl)-6-(1-methoxyethyl)pyridin-3-yl)piperazine-1-carboxylate (10.0 g, 11.3 mmol) in DMF (100 mL) at room temperature were added Cs₂CO₃ (5.54 g, 17.0 mmol) and iodoethane (3.54 g, 22.7 mmol). The resulting mixture was stirred for 8 h at 40° C. then diluted with H₂O (150 mL). The aqueous mixture was extracted with EtOAc (2×100 mL), and the combined organic extracts were washed with brine (3×50 mL), dried over Na₂SO₄, filtered, and concentrated under reduced pressure to afford benzyl (S)-4-(5-(5-bromo-3-(3-((tert-butyldiphenylsilyl)oxy)-2,2-difluoropropyl)-1-ethyl-1H-indol-2-yl)-6-(1-methoxyethyl)pyridin-3-yl)piperazine-1-carboxylate (10.2 g, crude), which was used without further purification. LCMS (ESI) m/z: [M+H] calcd for C₄₉H₅₅BrF₂N₄O₄Si: 909.3; found 909.5.

Step 5. To a solution of benzyl (S)-4-(5-(5-bromo-3-(3-((tert-butyldiphenylsilyl)oxy)-2,2-difluoropropyl)-1-ethyl-1H-indol-2-yl)-6-(1-methoxyethyl)pyridin-3-yl)piperazine-1-carboxylate (10.0 g, crude) in THE (100 mL) at room temperature was added a solution of 1M TBAF (100 mL, 100 mmol) in THF. The resulting mixture was stirred for 6 h at 60° C., then diluted with H₂O (100 mL). The aqueous mixture was extracted with EtOAc (2×50 mL), and the combined organic extracts were dried over Na₂SO₄, filtered, and concentrated under reduced pressure. Purification by normal phase chromatography afforded the desired atropisomer benzyl (S)-4-(5-(5-bromo-3-(2,2-difluoro-3-hydroxypropyl)-1-ethyl-1H-indol-2-yl)-6-(1-methoxyethyl)pyridin-3-yl)piperazine-1-carboxylate (1.60 g, 22% yield, M-atropisomer) and the undesired product (1.20 g, 16% yield) both as red solids. LCMS (ESI) m/z: [M+H] calcd for C₃₃H₃₇BrF₂N₄O₄: 671.2; found 671.1.

Step 6. To a solution of benzyl (S)-4-(5-(5-bromo-3-(2,2-difluoro-3-hydroxypropyl)-1-ethyl-1H-indol-2-yl)-6-(1-methoxyethyl)pyridin-3-yl)piperazine-1-carboxylate (1.6 g, 2.38 mmol), methyl (S)-1-((S)-2-((tert-butoxycarbonyl)amino)-3-(3-(4,4,5,5-tetramethyl-1,3,2-dioxaborolan-2-yl)-5-((triisopropylsilyl)oxy)phenyl)propanoyl)hexahydropyridazine-3-carboxylate (1.74 g, 2.86 mmol) and K₃PO₄ (1.01 g, 4.76 mmol) in dioxane (15 mL) and H₂O (3.0 mL) at room temperature was added Pd(dppf)Cl₂·DCM (190 mg, 0.238 mmol). The resulting mixture was stirred for 3 h at 70° C. then diluted with H₂O (20 mL). The aqueous mixture was extracted with EtOAc (2×40 mL), and the combined organic extracts were dried over Na₂SO₄, filtered, and concentrated under reduced pressure. Purification by normal phase chromatography afforded methyl (S)-1-((S)-3-(3-(2-(5-(4-((benzyloxy)carbonyl)piperazin-1-yl)-2-((S)-1-methoxyethyl)pyridin-3-yl)-3-(2,2-difluoro-3-hydroxypropyl)-1-ethyl-1H-indol-5-yl)-5-((triisopropylsilyl)oxy)phenyl)-2-((tert-butoxycarbonyl)amino)propanoyl)hexahydropyridazine-3-carboxylate (1.10 g, 40% yield) as a grey solid. LCMS (ESI) m/z: [M+H] calcd for C₆₂H₈₅F₂N₇O₁₀Si: 1154.6; found 1154.8.

Step 7. To a solution of methyl (S)-1-((S)-3-(3-(2-(5-(4-((benzyloxy)carbonyl)piperazin-1-yl)-2-((S)-1-methoxyethyl)pyridin-3-yl)-3-(2,2-difluoro-3-hydroxypropyl)-1-ethyl-1H-indol-5-yl)-5-((triisopropylsilyl)oxy)phenyl)-2-((tert-butoxycarbonyl)amino)propanoyl)hexahydropyridazine-3-carboxylate (1.0 g, 0.87 mmol) in THE (10 mL) and H₂O (10 mL) at 0° C. was added LiOH·H₂O (73 mg, 1.73 mmol). The resulting mixture was stirred for 2 h at 0° C., acidified to pH 6 with 2 M aq. HCl, and extracted with EtOAc (3×40 mL). The combined organic extracts were dried over Na₂SO₄, filtered, and concentrated under reduced pressure to afford (S)-1-((S)-3-(3-(2-(5-(4-((benzyloxy)carbonyl)piperazin-1-yl)-2-((S)-1-methoxyethyl)pyridin-3-yl)-3-(2,2-difluoro-3-hydroxypropyl)-1-ethyl-1H-indol-5-yl)-5-((triisopropylsilyl)oxy)phenyl)-2-((tert-butoxycarbonyl)amino)propanoyl)hexahydropyridazine-3-carboxylic acid (1.10 g, crude) which was used without further purification. LCMS (ESI) m/z: [M+H] calcd for C₆₁H₈₃F₂N₇O₁₀Si: 1140.6; found 1140.6.

Step 8. To a solution of (S)-1-((S)-3-(3-(2-(5-(4-((benzyloxy)carbonyl)piperazin-1-yl)-2-((S)-1-methoxyethyl)pyridin-3-yl)-3-(2,2-difluoro-3-hydroxypropyl)-1-ethyl-1H-indol-5-yl)-5-((triisopropylsilyl)oxy)phenyl)-2-((tert-butoxycarbonyl)amino)propanoyl)hexahydropyridazine-3-carboxylic acid (1.10 g, crude) in DCM (30 mL) at room temperature were added DIPEA (2.49 g, 19.3 mmol), HOBT (651 mg, 4.83 mmol), and EDCI (2.77 g, 14.5 mmol). The resulting mixture was stirred overnight at room temperature then quenched by the addition of H₂O (100 mL). The aqueous layer was extracted with DCM (3×50 mL), and the combined organic extracts were washed with brine (50 mL), dried over Na₂SO₄, filtered, concentrated under reduced pressure. Purification by normal phase chromatography afforded benzyl 4-(5-((6³S,4S)-4-((tert-butoxycarbonyl)amino)-1¹-ethyl-10,10-difluoro-5,7-dioxo-2⁵-((triisopropylsilyl)oxy)-6¹,6²,6³,6⁴,6⁵,6⁶-hexahydro-1¹H-8-oxa-1(5,3)-indola-6(1,3)-pyridazina-2(1,3)-benzenacycloundecaphane-1²-yl)-6-((S)-1-methoxyethyl)pyridin-3-yl)piperazine-1-carboxylate (900 mg, 75% yield over 2 steps) as a yellow solid. LCMS (ESI) m/z: [M+H] calcd for C₆₁H₅₁F₂N₇O₉Si: 1122.6; found 1122.6.

Step 9. To a solution of benzyl 4-(5-((6³S,4S)-4-((tert-butoxycarbonyl)amino)-1¹-ethyl-10,10-difluoro-5,7-dioxo-2⁵-((triisopropylsilyl)oxy)-6¹,6²,6³,6⁴,6⁵,6⁶-hexahydro-1¹H-8-oxa-1(5,3)-indola-6(1,3)-pyridazina-2(1,3)-benzenacycloundecaphane-1²-yl)-6-((S)-1-methoxyethyl)pyridin-3-yl)piperazine-1-carboxylate (900 mg, 0.802 mmol) in DMF (9.0 mL) at room temperature was added CsF (244 mg, 1.60 mmol). The resulting mixture was stirred overnight at room temperature then quenched by the addition of H₂O. The aqueous mixture was extracted with EtOAc (3×20 mL), and the combined organic extracts were washed with brine (20 mL), dried over Na₂SO₄, filtered, and concentrated under reduced pressure to afford benzyl 4-(5-((6³S,4S)-4-((tert-butoxycarbonyl)amino)-1¹-ethyl-10,10-difluoro-2⁵-hydroxy-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-12-yl)-6-((S)-1-methoxyethyl)pyridin-3-yl)piperazine-1-carboxylate (800 mg, crude), which was used without further purification. LCMS (ESI) m/z: [M+H] calcd for C₅₂H₆₁F₂N₇O₉: 966.5; found 966.6.

Step 10. To a solution of benzyl 4-(5-((6³S,4S)-4-((tert-butoxycarbonyl)amino)-1¹-ethyl-10,10-difluoro-2⁵-hydroxy-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-12-yl)-6-((S)-1-methoxyethyl)pyridin-3-yl)piperazine-1-carboxylate (850 mg, crude) in DCM (9.0 mL) at 0° C. was added TFA (3.0 mL). The resulting mixture was stirred for 2 h at 0° C., concentrated under reduced pressure, and the concentrate basified to pH 8 with sat. aq. NaHCO₃. The aqueous phase was extracted with EtOAc (2×40 mL), and the combined organic extracts were dried over Na₂SO₄, filtered, and concentrated under reduced pressure to afford benzyl 4-(5-((6³S,4S)-4-amino-1′-ethyl-10,10-difluoro-2⁵-hydroxy-5,7-dioxo-6¹,6²,6³,6⁴,6⁵,6⁶-hexahydro-1¹H-8-oxa-1(5,3)-indola-6(1,3)-pyridazina- 2(1,3)-benzenacycloundecaphane-1²-yl)-6-((S)-1-methoxyethyl)pyridin-3-yl)piperazine-1-carboxylate (800 mg, crude), which was used without further purification. LCMS (ESI) m/z: [M+H] calcd for C₄₇H₅₃F₂N₇O₇: 866.4; found 844.5.

Step 11. To a solution of benzyl 4-(5-((6³S,4S)-4-amino-1′-ethyl-10,10-difluoro-2⁵-hydroxy-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-1²-yl)-6-((S)-1-methoxyethyl)pyridin-3-yl)piperazine-1-carboxylate (790 mg, crude) and N-((2S,3S)-1-(tert-butoxycarbonyl)-2-((tosyloxy)methyl)pyrrolidine-3-carbonyl)-N-methyl-L-valine (608 mg, 1.19 mmol) in DMF (15 mL) at 0° C. were added DIPEA (4.72 g, 36.5 mmol) and COMU (781 mg, 1.82 mmol). The resulting mixture was stirred for 3 h at 0° C. then quenched by the addition of H₂O (30 mL). The aqueous mixture was extracted with EtOAc (2×30 mL), and the combined organic extracts were washed with brine (50 mL), dried over Na₂SO₄, filtered, and concentrated under reduced pressure. Purification by normal phase chromatography afforded benzyl 4-(5-((6³S,4S)-4-((S)-2-((2S,3S)-1-(tert-butoxycarbonyl)-N-methyl-2-((tosyloxy)methyl)pyrrolidine-3-carboxamido)-3-methylbutanamido)-1¹-ethyl-10,10-difluoro-2⁵-hydroxy-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-1²-yl)-6-((S)-1-methoxyethyl)pyridin-3-yl)piperazine-1-carboxylate (980 mg, 88% yield over 3 steps) as a red solid. LCMS (ESI) m/z: [M+H] calcd for C₇₁H₈₇F₂N₉O₁₄S: 1360.6; found 1361.4.

Step 12. To a solution of benzyl 4-(5-((6³S,4S)-4-((S)-2-((2S,3S)-1-(tert-butoxycarbonyl)-N-methyl-2-((tosyloxy)methyl)pyrrolidine-3-carboxamido)-3-methylbutanamido)-1¹-ethyl-10,10-difluoro-2⁵-hydroxy-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-1²-yl)-6-((S)-1-methoxyethyl)pyridin-3-yl)piperazine-1-carboxylate (980 mg, 0.824 mmol) in DMF (98 mL) at room temperature were added KI (137 mg, 0.824 mmol) and K₂CO₃ (1.14 g, 8.240 mmol). The resulting mixture was stirred for 3 h at 80° C. then diluted with H₂O (200 mL). The aqueous mixture was extracted with EtOAc (2×100 mL), and the combined organic extracts were washed with brine (2×100 mL), dried over Na₂SO₄, and concentrated under reduced pressure. Purification by normal phase chromatography afforded tert-butyl (3aS,6S,9S,15S,32aS)-21-(5-(4-((benzyloxy)carbonyl)piperazin-1-yl)-2-((S)-1-methoxyethyl)pyridin-3-yl)-22-ethyl-19,19-difluoro-6-isopropyl-5-methyl-4,7,10,16-tetraoxo-2,3,3a,4,5,6,7,8,9,10,13,14,15,16,18,19,20,22,32,32a-icosahydro-1H,12H-11,15-epimino-23,25-etheno-9,28-methano-26,30-(metheno)dipyrrolo[2,3-c:3′,4′-v][1,18]dioxa[6,9,12]triazacyclotriacontine-1-carboxylate (448 mg, 48% yield) as a red solid. LCMS (ESI) m/z: [M+H] calcd for C₆₄H₇₉F₂N₉O₁₁: 1188.6; found 1188.5.

Step 13. To a solution of tert-butyl (3aS,6S,9S,15S,32aS)-21-(5-(4-((benzyloxy)carbonyl)piperazin-1-yl)-2-((S)-1-methoxyethyl)pyridin-3-yl)-22-ethyl-19,19-difluoro-6-isopropyl-5-methyl-4,7,10,16-tetraoxo-2,3,3a,4,5,6,7,8,9,10,13,14,15,16,18,19,20,22,32,32a-icosahydro-1H,12H-11,15-epimino-23,25-etheno-9,28-methano-26,30-(metheno)dipyrrolo[2,3-c:3′,4′-v][1,18]dioxa[6,9,12]triazacyclotriacontine-1-carboxylate (300 mg, 0.252 mmol) in IPA (5.0 mL) at room temperature was added Pd(OH)₂/C (71 mg). The resulting mixture was stirred for 16 h at 40° C. under an atmosphere of H₂, filtered, and the filter cake washed with 10:1 vol. CHCl₃:IPA (2×30 mL). The filtrate was concentrated under reduced pressure to afford tert-butyl (3aS,6S,9S,15S,32aS)-22-ethyl-19,19-difluoro-6-isopropyl-21-(2-((S)-1-methoxyethyl)-5-(piperazin-1-yl)pyridin-3-yl)-5-methyl-4,7,10,16-tetraoxo-2,3,3a,4,5,6,7,8,9,10,13,14,15,16,18,19,20,22,32,32a-icosahydro-1H,12H-11,15-epimino-23,25-etheno-9,28-methano-26,30-(metheno)dipyrrolo[2,3-c:3′,4′-v][1,18]dioxa[6,9,12]triazacyclotriacontine-1-carboxylate (238 mg, crude), which was used without further purification. LCMS (ESI) m/z: [M+H] calcd for C₅₆H₇₃F₂N₉O₉: 1054.5; found 1055.0.

Step 14. To a solution of tert-butyl (3aS,6S,9S,15S,32aS)-22-ethyl-19,19-difluoro-6-isopropyl-21-(2-((S)-1-methoxyethyl)-5-(piperazin-1-yl)pyridin-3-yl)-5-methyl-4,7,10,16-tetraoxo-2,3,3a,4,5,6,7,8,9,10,13,14,15,16,18,19,20,22,32,32a-icosahydro-1H,12H-11,15-epimino-23,25-etheno-9,28-methano-26,30-(metheno)dipyrrolo[2,3-c:3′,4′-v][1,18]dioxa[6,9,12]triazacyclotriacontine-1-carboxylate (238 mg, crude) in IPA (4.0 mL) at 0° C. were added CH₃CO₂H (40.7 mg, 0.678 mmol), (1-ethoxycyclopropoxy)trimethylsilane (118 mg, 0.678 mmol) and NaBH₃CN (43 mg, 0.678 mmol). The resulting mixture was stirred for 16 h at 60° C. then neutralized to pH 7 with sat. aq. NaHCO₃. The aqueous mixture was extracted with EtOAc (2×20 mL), and the combined organic extracts were dried over Na₂SO₄, filtered, and concentrated under reduced pressure to afford tert-butyl (3aS,6S,9S,15S,32aS)-21-(5-(4-cyclopropylpiperazin-1-yl)-2-((S)-1-methoxyethyl)pyridin-3-yl)-22-ethyl-19,19-difluoro-6-isopropyl-5-methyl-4,7,10,16-tetraoxo-2,3,3a,4,5,6,7,8,9,10,13,14,15,16,18,19,20,22,32,32a-icosahydro-1H,12H-11,15-epimino-23,25-etheno-9,28-methano-26,30-(metheno)dipyrrolo[2,3-c:3′,4′-v][1,18]dioxa[6,9,12]triazacyclotriacontine-1-carboxylate (205 mg, crude) which was used without further purification. LCMS (ESI) m/z: [M+H] calcd for C₅₉H₇₇F₂N₉O₉: 1094.6; found 1094.6.

Step 15. To a solution of tert-butyl (3aS,6S,9S,15S,32aS)-21-(5-(4-cyclopropylpiperazin-1-yl)-2-((S)-1-methoxyethyl)pyridin-3-yl)-22-ethyl-19,19-difluoro-6-isopropyl-5-methyl-4,7,10,16-tetraoxo-2,3,3a,4,5,6,7,8,9,10,13,14,15,16,18,19,20,22,32,32a-icosahydro-1H,12H-11,15-epimino-23,25-etheno-9,28-methano-26,30-(metheno)dipyrrolo[2,3-c:3′,4′-v][1,18]dioxa[6,9,12]triazacyclotriacontine-1-carboxylate (185 mg, crude) in DCM (3.0 mL) at 0° C. was added TFA (1.0 mL). The resulting mixture was stirred for 2 h at 0° C., concentrated under reduced pressure, and the concentrate neutralized to pH 7 with sat. aq. NaHCO₃. The aqueous layer was extracted with EtOAc (2×40 mL), and the combined organic extracts were dried over Na₂SO₄, filtered, and concentrated under reduced pressure to afford (3aS,6S,9S,15S,32aS)-21-(5-(4-cyclopropylpiperazin-1-yl)-2-((S)-1-methoxyethyl)pyridin-3-yl)-22-ethyl-19,19-difluoro-6-isopropyl-5-methyl-3,3a,5,6,8,9,14,15,18,19,20,22,32,32a-tetradecahydro-1H,12H-11,15-epimino-23,25-etheno-9,28-methano-26,30-(metheno)dipyrrolo[2,3-c:3′,4′-v][1,18]dioxa[6,9,12]triazacyclotriacontine-4,7,10,16(2H,13H)-tetraone (155 mg, crude), which was used without further purification. LCMS (ESI) m/z: [M+H] calcd for C₅₄H₆₉F₂N₉O₇: 994.5; found 994.6.

Step 16. To a solution of (3aS,6S,9S,15S,32aS)-21-(5-(4-cyclopropylpiperazin-1-yl)-2-((S)-1-methoxyethyl)pyridin-3-yl)-22-ethyl-19,19-difluoro-6-isopropyl-5-methyl-3,3a,5,6,8,9,14,15,18,19,20,22,32,32a-tetradecahydro-1H,12H-11,15-epimino-23,25-etheno-9,28-methano-26,30-(metheno)dipyrrolo[2,3-c:3′,4′-v][1,18]dioxa[6,9,12]triazacyclotriacontine-4,7,10,16(2H,13H)-tetraone (155 mg, crude) in DCM (3.0 mL) at 0° C. were added DIPEA (202 mg, 1.56 mmol), 2-fluoro-2-methylpropanoic acid (21.5 mg, 0.203 mmol) and COMU (134 mg, 0.312 mmol). The resulting mixture was stirred for 1 h at 0° C. then quenched by the addition of H₂O (20 mL). The aqueous layer was extracted with DCM (2×30 mL), and the combined organic extracts were dried over Na₂SO₄, filtered, and concentrated under reduced pressure. Purification by reversed phase chromatography afforded (3aS,6S,9S,15S,32aS)-21-(5-(4-cyclopropylpiperazin-1-yl)-2-((S)-1-methoxyethyl)pyridin-3-yl)-22-ethyl-19,19-difluoro-1-(2-fluoro-2-methylpropanoyl)-6-isopropyl-5-methyl-3,3a,5,6,8,9,14,15,18,19,20,22,32,32a-tetradecahydro-1H,12H-11,15-epimino-23,25-etheno-9,28-methano-26,30-(metheno)dipyrrolo[2,3-c:3′,4′-v][1,18]dioxa[6,9,12]triazacyclotriacontine-4,7,10,16(2H,13H)-tetraone (38 mg, 15% yield over 4 steps). LCMS (ESI) m/z: [M+H] calcd for C₅₈H₇₄F₃N₉O₈: 1082.6; found 1082.7. ¹H NMR (400 MHz, DMSO-d₆) δ 8.49-8.40 (m, 2H), 8.14 (d, J=8.1 Hz, 1H), 7.99 (s, 1H), 7.78-7.71 (m, 1H), 7.64-7.57 (m, 2H), 7.32-7.24 (m, 3H), 6.85 (s, 1H), 5.37 (t, J=9.2 Hz, 2H), 5.29 (d, J=12.5 Hz, 2H), 4.69 (d, J=11.0 Hz, 2H), 4.45 (s, 2H), 4.10 (s, 2H), 3.94 (dd, J=12.8, 6.5 Hz, 3H), 3.54 (s, 1H), 3.22 (s, 4H), 3.05 (s, 2H), 2.97-2.90 (m, 2H), 2.88-2.76 (m, 3H), 2.67 (t, J=5.0 Hz, 4H), 2.38 (s, 2H), 2.28-2.23 (m, 1H), 2.00 (t, J=5.3 Hz, 1H), 1.83 (s, 1H), 1.66-1.51 (m, 7H), 1.49 (d, J=3.2 Hz, 1H), 1.34-1.18 (m, 6H), 1.02 (t, J=7.1 Hz, 3H), 0.92-0.74 (m, 5H), 0.68 (d, J=6.8 Hz, 3H), 0.47-0.40 (m, 2H), 0.34 (d, J=3.7 Hz, 2H).

### Example A345. Synthesis of (3aS,6S,9S,15S,32aS)-21-(5-(1-cyclopropylpiperidin-4-yl)-2-((S)-1-methoxyethyl)pyridin-3-yl)-22-ethyl-1-(2-fluoro-2-methylpropanoyl)-5,19,19-trimethyl-6-((R)-tetrahydrofuran-3-yl)-3,3a,5,6,8,9,14,15,18,19,20,22,32,32a-tetradecahydro-1H,12H-11,15-epimino-23,25-etheno-9,28-methano-26,30-(metheno)dipyrrolo[2,3-c:3′,4′-v][1,18]dioxa[6,9,12]triazacyclotriacontine-4,7,10,16(2H,13H)-tetraone

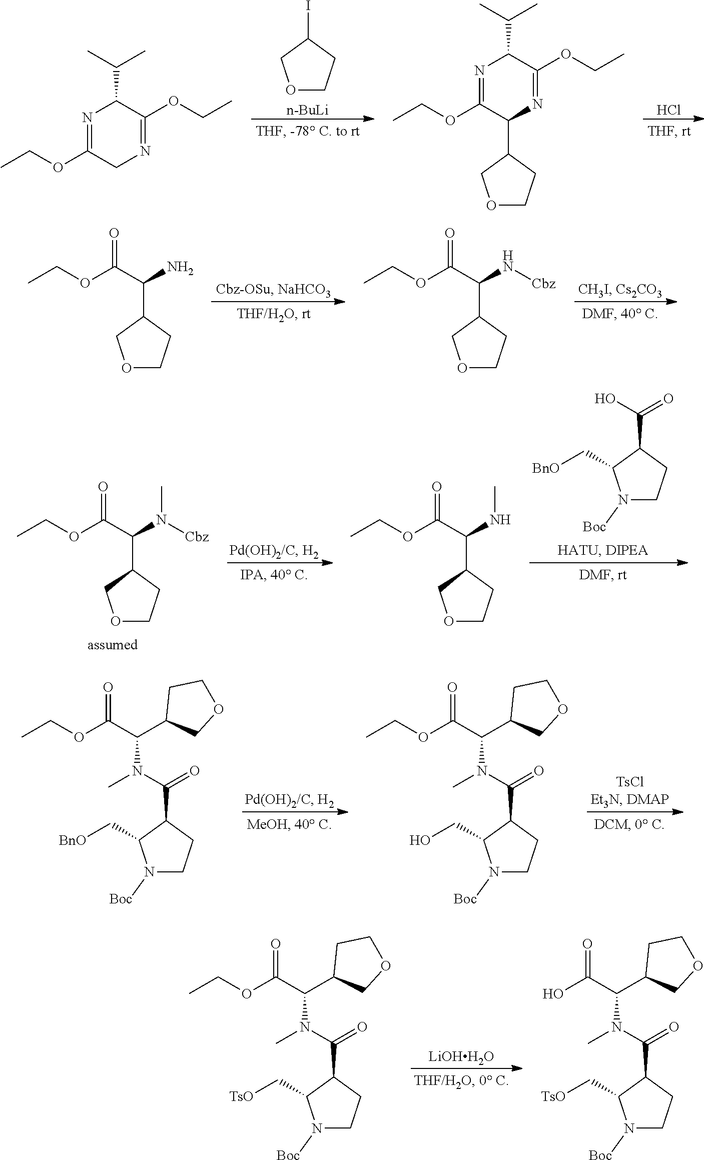

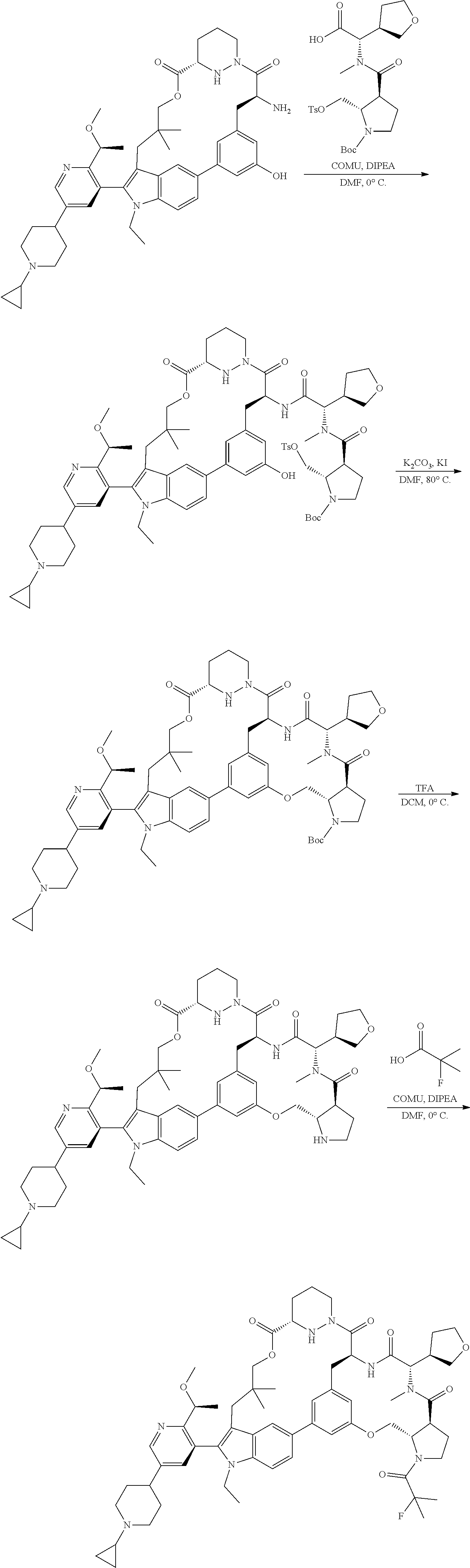

Step 1. To a solution of (R)-3,6-diethoxy-2-isopropyl-2,5-dihydropyrazine (17.7 g, 83.3 mmol) in THF (120 mL) stirred at −78° C. was added a solution of n-BuLi (39.4 mL, 98.5 mmol, 2.5 M in hexanes). The resulting mixture was stirred for 2 h and was followed by the addition of a solution of 3-iodotetrahydrofuran (15 g, 75.9 mmol) in THF (30 mL). The reaction mixture was stirred overnight at room temperature and was then quenched at 0° C. by the addition of sat. aq. NH₄Cl (200 mL). The aqueous mixture was extracted with EtOAc (2×200 mL), and the combined organic extracts were dried over Na₂SO₄, filtered, and concentrated under reduced pressure. Purification by reversed phase chromatography afforded (2R,5S)-3,6-diethoxy-2-isopropyl-5-(tetrahydrofuran-3-yl)-2,5-dihydropyrazine (13 g, 64% yield) as a yellow oil. LCMS (ESI) m/z: [M+H] calcd for C₁₅H₂₆N₂O₃: 283.2; found 283.3.

Step 2. To a solution of (2R,5S)-3,6-diethoxy-2-isopropyl-5-(tetrahydrofuran-3-yl)-2,5-dihydropyrazine (9.0 g, 31.9 mmol) in THE (50 mL) stirred at 0° C. was added 1M aq. HCl (50 mL). The resulting mixture was stirred overnight at room temperature and was then basified to pH=8 by the addition of sat. aq. NaHCO₃. The aqueous mixture was extracted with EtOAc (3×200 mL), and the combined organic extracts were dried over Na₂SO₄, filtered, and concentrated under reduced pressure to afford ethyl (2S)-2-amino-2-(tetrahydrofuran-3-yl)acetate (9.0 g, crude) which was taken directly to the next reaction without further purification. LCMS (ESI) m/z: [M+H] calcd for C₈H₁₅NO₃: 174.1; found 174.1.

Step 3. To a solution of ethyl (2S)-2-amino-2-(tetrahydrofuran-3-yl)acetate (9.0 g, crude) and NaHCO₃ (13.1 g, 155 mmol) in THE (100 mL) stirred at 0° C. was added H₂O (100 mL) and benzyl (2,5-dioxopyrrolidin-1-yl) carbonate (19.4 g, 77.9 mmol). The resulting mixture was stirred for 2 h at room temperature and was then extracted with EtOAc (3×100 mL). The combined organic extracts were dried over Na₂SO₄, filtered, and concentrated under reduced pressure. Purification by normal phase chromatography afforded ethyl (2S)-2-(((benzyloxy)carbonyl)amino)-2-(tetrahydrofuran-3-yl)acetate (11.5 g, 72.0% yield) as a yellow oil. LCMS (ESI) m/z: [M+H] calcd for C₁₆H₂₁NO₅: 308.2; found 308.2.

Step 4. To a solution of ethyl (2S)-2-(((benzyloxy)carbonyl)amino)-2-(tetrahydrofuran-3-yl)acetate (11.5 g, 37.4 mmol) and Cs₂CO₃ (61.0 g, 187 mmol) in DMF (200 mL) stirred at 0° C. was added iodomethane (21.2 g, 149 mmol). The resulting mixture was stirred overnight at 40° C. and quenched by the addition of H₂O (200 mL). The aqueous mixture was extracted with EtOAc (3×200 mL), and the combined organic extracts were washed with brine (3×100 mL), dried over Na₂SO₄, filtered, and concentrated under reduced pressure. Purification by reversed phase chromatography afforded a mixture of two diastereomers, which were separated by preparative-SFC to afford the faster-eluting ethyl (S)-2-(((benzyloxy)carbonyl)(methyl)amino)-2-((R)-tetrahydrofuran-3-yl)acetate (2.7 g, 22% yield, assumed configuration) and the slower-eluting ethyl (S)-2-(((benzyloxy)carbonyl)(methyl)amino)-2-((S)-tetrahydrofuran-3-yl)acetate (2.2 g, 41% yield, assumed configuration). LCMS (ESI) m/z: [M+H] calcd for C₁₇H₂₃NO₅: 322.2; found 322.1.

Step 5. To a solution of ethyl (S)-2-(((benzyloxy)carbonyl)(methyl)amino)-2-((R)-tetrahydrofuran-3-yl)acetate (500 mg, 1.56 mmol) in IPA (10 mL) was added 20 wt. % Pd(OH)₂/C (437 mg). The resulting mixture was stirred for 2 h at 40° C. under an atmosphere of H₂, filtered, and the filter cake washed with EtOAc (3×10 mL). The filtrate was concentrated under reduced pressure to afford ethyl (S)-2-(methylamino)-2-((R)-tetrahydrofuran-3-yl)acetate (300 mg, crude) which was used in the next reaction without further purification. LCMS (ESI) m/z: [M+H] calcd for C₉H₁₇NO₃: 188.1; found 188.2.

Step 6. To a solution of ethyl (S)-2-(methylamino)-2-((R)-tetrahydrofuran-3-yl)acetate (260 mg, 1.39 mmol) and (2S,3S)-2-((benzyloxy)methyl)-1-(tert-butoxycarbonyl)pyrrolidine-3-carboxylic acid (512 mg, 1.53 mmol) in DMF (5.0 mL) stirred at 0° C. was added DIPEA (1.79 g, 13.9 mmol) and HATU (1.06 g, 2.78 mmol). The resulting mixture was stirred for 2 h at room temperature and was then quenched by the addition of H₂O (10 mL). The aqueous mixture was extracted with EtOAc (3×10 mL), and the combined organic extracts were washed with brine (3×10 mL), dried over Na₂SO₄, filtered, and concentrated under reduced pressure. Purification by normal phase chromatography afforded tert-butyl (2S,3S)-2-((benzyloxy)methyl)-3-(((S)-2-ethoxy-2-oxo-1-((R)-tetrahydrofuran-3-yl)ethyl)(methyl)carbamoyl)pyrrolidine-1-carboxylate (400 mg, 58% yield over 2 steps) as a yellow solid. LCMS (ESI) m/z: [M+H] calcd for C₂₇H₄₀N₂O₇: 505.3; found 505.3.

Step 7. To a solution of tert-butyl (2S,3S)-2-((benzyloxy)methyl)-3-(((S)-2-ethoxy-2-oxo-1-((R)-tetrahydrofuran-3-yl)ethyl)(methyl)carbamoyl)pyrrolidine-1-carboxylate (400 mg, 0.793 mmol) in MeOH (5.0 mL) stirred at room temperature was added 20 wt. % Pd(OH)₂/C (200 mg). The resulting mixture was stirred for 3 h at 40° C. under an atmosphere of H₂, filtered, and the filter cake washed with 10:1 vol. DCM/MeOH (3×20 mL). The filtrate was concentrated under reduced pressure to afford tert-butyl (2S,3S)-3-(((S)-2-ethoxy-2-oxo-1-((R)-tetrahydrofuran-3-yl)ethyl)(methyl)carbamoyl)-2-(hydroxymethyl)pyrrolidine-1-carboxylate (380 mg, crude) which was used in the next reaction without further purification. LCMS (ESI) m/z: [M+Na] calcd for C₂₀H₃₄N₂O₇: 437.2; found 437.1.

Step 8. To a solution of tert-butyl (2S,3S)-3-(((S)-2-ethoxy-2-oxo-1-((R)-tetrahydrofuran-3-yl)ethyl)(methyl)carbamoyl)-2-(hydroxymethyl)pyrrolidine-1-carboxylate (370 mg, crude) in DCM (4.0 mL) stirred at 0° C. was added Et₃N (271 mg, 2.68 mmol), p-toluenesulfonyl chloride (340 mg, 1.79 mmol), and DMAP (10.9 mg, 0.089 mmol). The resulting mixture was stirred for 2 h at 0° C. and quenched by the addition of H₂O (30 mL). The aqueous phase was extracted with DCM (2×20 mL), and the combined organic extracts were dried over Na₂SO₄, filtered, and concentrated under reduced pressure. Purification by normal phase chromatography afforded tert-butyl (2S,3S)-3-(((S)-2-ethoxy-2-oxo-1-((R)-tetrahydrofuran-3-yl)ethyl)(methyl)carbamoyl)-2-((tosyloxy)methyl)pyrrolidine-1-carboxylate (340 mg, 78% yield over 2 steps) as a clear oil. LCMS (ESI) m/z: [M+H] calcd for C₂₇H₄₀N₂O₉S: 569.3; found 569.2.

Step 9. To a solution of tert-butyl (2S,3S)-3-(((S)-2-ethoxy-2-oxo-1-((R)-tetrahydrofuran-3-yl)ethyl)(methyl)carbamoyl)-2-((tosyloxy)methyl)pyrrolidine-1-carboxylate (330 mg, 0.580 mmol) in THE (2.0 mL) and H₂O (2.0 mL) stirred at 0° C. was added LiOH·H₂O (27.8 mg, 1.16 mmol). The resulting mixture was stirred for 1 h, acidified to pH=6 by the addition of 2 M aq. HCl, and diluted with H₂O (40 mL). The aqueous mixture was extracted with EtOAc (2×20 mL), and the combined organic extracts were dried over Na₂SO₄, filtered, and concentrated under reduced pressure to afford (S)-2-((2S,3S)-1-(tert-butoxycarbonyl)-N-methyl-2-((tosyloxy)methyl)pyrrolidine-3-carboxamido)-2-((R)-tetrahydrofuran-3-yl)acetic acid (318 mg, crude) which was used in the next reaction without further purification. LCMS (ESI) m/z: [M+Na] calcd for C₂₅H₃₆N₂O₉S: 563.2; found 563.1.

Step 10. To a solution of (6³S,4S)-4-amino-1²-(5-(1-cyclopropylpiperidin-4-yl)-2-((S)-1-methoxyethyl)pyridin-3-yl)-1¹-ethyl-2⁵-hydroxy-10,10-dimethyl-6¹,6²,6³,6⁴,6⁵,6⁶-hexahydro-1 ¹H-8-oxa-1(5,3)-indola-6(1,3)-pyridazina-2(1,3)-benzenacycloundecaphane-5,7-dione (300 mg, 0.393 mmol) in DMF (5.0 mL) stirred at 0° C. were added DIPEA (2.03 g, 15.7 mmol), (S)-2-((2S,3S)-1-(tert-butoxycarbonyl)-N-methyl-2-((tosyloxy)methyl)pyrrolidine-3-carboxamido)-2-((R)-tetrahydrofuran-3-yl)acetic acid (319 mg, 0.590 mmol) and COMU (337 mg, 0.786 mmol). The resulting mixture was stirred for 2 h at 0° C. and quenched by the addition of H₂O (20 mL). The aqueous mixture was extracted with EtOAc (2×20 mL), and the combined organic extracts were dried over Na₂SO₄, filtered, and concentrated under reduced pressure. Purification by normal phase chromatography afforded tert-butyl (2S,3S)-3-(((1 S)-2-(((6³S,4S)-1²-(5-(1-cyclopropylpiperidin-4-yl)-2-((S)-1-methoxyethyl)pyridin-3-yl)-1¹-ethyl-2⁵-hydroxy-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)amino)-2-oxo-1-((R)-tetrahydrofuran-3-yl)ethyl)(methyl)carbamoyl)-2-((tosyloxy)methyl)pyrrolidine-1-carboxylate (340 mg, 67% yield) as a red solid. LCMS (ESI) m/z: [M+H] calcd for C₇₀H₉₂N₈O₁₃S: 1285.7; found 1285.6.

Step 11. To a solution of tert-butyl (2S,3S)-3-(((1 S)-2-(((6³S,4S)-1²-(5-(1-cyclopropylpiperidin-4-yl)-2-((S)-1-methoxyethyl)pyridin-3-yl)-1¹-ethyl-2⁵-hydroxy-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)amino)-2-oxo-1-((R)-tetrahydrofuran-3-yl)ethyl)(methyl)carbamoyl)-2-((tosyloxy)methyl)pyrrolidine-1-carboxylate (340 mg, 0.264 mmol) in DMF (34 mL) stirred at room temperature were added K₂CO₃ (366 mg, 2.640 mmol) and KI (43.9 mg, 0.264 mmol). The resulting mixture was stirred for 2 h at 80° C. and quenched by the addition of H₂O (100 mL). The aqueous mixture was extracted with EtOAc (2×50 mL), and the combined organic extracts were dried over Na₂SO₄, filtered, and concentrated under reduced pressure. Purification by normal phase chromatography afforded tert-butyl (3aS,6S,9S,15S,32aS)-21-(5-(1-cyclopropylpiperidin-4-yl)-2-((S)-1-methoxyethyl)pyridin-3-yl)-22-ethyl-5,19,19-tri methyl-4,7,10,16-tetraoxo-6-((R)-tetrahydrofuran-3-yl)-2,3,3a,4,5,6,7,8,9,10,13,14,15,16,18,19,20,22,32,32a-icosahydro-1H,12H-11,15-epimino-23,25-etheno-9,28-methano-26,30-(metheno)dipyrrolo[2,3-c:3′,4′-v][1,18]dioxa[6,9,12]triazacyclotriacontine-1-carboxylate (220 mg, 67% yield) as a red solid. LCMS (ESI) m/z: [M+H] calcd for C₆₃H₈₄N₈O₁₀: 1113.6; found 1114.2.

Step 12. To a solution of tert-butyl (3aS,6S,9S,15S,32aS)-21-(5-(1-cyclopropylpiperidin-4-yl)-2-((S)-1-methoxyethyl)pyridin-3-yl)-22-ethyl-5,19,19-trimethyl-4,7,10,16-tetraoxo-6-((R)-tetrahydrofuran-3-yl)-2,3,3a,4,5,6,7,8,9,10,13,14,15,16,18,19,20,22,32,32a-icosahydro-1H,12H-11,15-epimino-23,25-etheno-9,28-methano-26,30-(metheno)dipyrrolo[2,3-c:3′,4′-v][1,18]dioxa[6,9,12]triazacyclotriacontine-1-carboxylate (210 mg, 0.189 mmol) in DCM (6.0 mL) stirred at 0° C. was added TFA (2.0 mL). The resulting mixture was stirred for 1 h at 0° C., concentrated under reduced pressure, and the concentrate diluted with sat. aq. NaHCO₃ (50 mL). The aqueous mixture was extracted with EtOAc (2×20 mL), and the combined organic layers were dried over Na₂SO₄, filtered, and concentrated under reduced pressure to afford (3aS,6S,9S,15S,32aS)-21-(5-(1-cyclopropylpiperidin-4-yl)-2-((S)-1-methoxyethyl)pyridin-3-yl)-22-ethyl-5,19,19-trimethyl-6-((R)-tetrahydrofuran-3-yl)-3,3a,5,6,8,9,14,15,18,19,20,22,32,32a-tetradecahydro-1H,12H-11,15-epimino-23,25-etheno-9,28-methano-26,30-(metheno)dipyrrolo[2,3-c:3′,4′-v][1,18]dioxa[6,9,12]triazacyclotriacontine-4,7,10,16(2H,13H)-tetraone (170 mg, crude) which was taken directly to the next reaction without further purification. LCMS (ESI) m/z: [M+H] calcd for C₅₈H₇₆N₈O₈: 1013.6; found 1013.8.

Step 13. To a solution of (3aS,6S,9S,15S,32aS)-21-(5-(1-cyclopropylpiperidin-4-yl)-2-((S)-1-methoxyethyl)pyridin-3-yl)-22-ethyl-5,19,19-trimethyl-6-((R)-tetrahydrofuran-3-yl)-3,3a,5,6,8,9,14,15,18,19,20,22,32,32a-tetradecahydro-1H,12H-11,15-epimino-23,25-etheno-9,28-methano-26,30-(metheno)dipyrrolo[2,3-c:3′,4′-v][1,18]dioxa[6,9,12]triazacyclotriacontine-4,7,10,16(2H,13H)-tetraone (170 mg, crude) in DCM (3.0 mL) stirred at 0° C. was added DIPEA (204 mg, 1.58 mmol), 2-fluoro-2-methylpropanoic acid (21.8 mg, 0.205 mmol), and COMU (87.9 mg, 0.205 mmol). The resulting mixture was stirred for 2 h at 0° C. and quenched by the addition of H₂O (80 mL). The aqueous mixture extracted with DCM (2×30 mL), and the combined organic extracts were dried over Na₂SO₄, filtered, and concentrated under reduced pressure. Purification by normal phase chromatography afforded (3aS,6S,9S,15S,32aS)-21-(5-(1-cyclopropylpiperidin-4-yl)-2-((S)-1-methoxyethyl)pyridin-3-yl)-22-ethyl-1-(2-fluoro-2-methylpropanoyl)-5,19,19-trimethyl-6-((R)-tetrahydrofuran-3-yl)-3,3a,5,6,8,9,14,15,18,19,20,22,32,32a-tetradecahydro-1H,12H-11,15-epimino-23,25-etheno-9,28-methano-26,30-(metheno)dipyrrolo[2,3-c:3′,4′-v][1,18]dioxa[6,9,12]triazacyclotriacontine-4,7,10,16(2H,13H)-tetraone (13.6 mg, 6.5% yield over 2 steps) as a white solid. LCMS (ESI) m/z: [M+H] calcd for C₆₂H₈₁FN₈O₉: 1101.6; found 1101.7; ¹H NMR (400 MHz, DMSO-d₆) δ 8.21 (d, J=7.7 Hz, 1H), 8.01 (s, 1H), 7.77-7.68 (m, 1H), 7.65 (d, J=2.3 Hz, 1H), 7.57 (dd, J=8.6, 4.7 Hz, 1H), 7.47 (s, 1H), 7.28 (s, 1H), 7.15 (s, 1H), 6.92 (d, J=6.7 Hz, 1H), 5.43 (d, J=12.5 Hz, 1H), 5.16 (s, 1H), 5.05-4.92 (m, 1H), 4.69 (d, J=8.3 Hz, 1H), 4.59 (d, J=11.6 Hz, 1H), 4.48 (d, J=11.3 Hz, 1H), 4.33-4.12 (m, 2H), 4.11-3.92 (m, 3H), 3.80-3.69 (m, 3H), 3.61 (dd, J=12.0, 7.6 Hz, 2H), 3.50 (d, J=10.5 Hz, 2H), 3.24 (s, 2H), 3.16 (dd, J=8.9, 6.0 Hz, 1H), 3.08-2.99 (m, 4H), 2.99-2.86 (m, 1H), 2.86-2.74 (m, 1H), 2.75 (s, 1H), 2.70 (s, 1H), 2.54 (s, 1H), 2.38-2.19 (m, 4H), 1.92-1.77 (m, 3H), 1.65 (s, 7H), 1.61 (d, J=6.1 Hz, 2H), 1.55 (d, J=5.3 Hz, 2H), 1.49 (d, J=1.5 Hz, 1H), 1.46-1.29 (m, 5H), 1.29-1.21 (m, 4H), 1.01 (t, J=7.1 Hz, 2H), 0.92 (s, 2H), 0.66 (s, 2H), 0.61 (s, 2H), 0.45-0.38 (m, 4H), 0.31 (s, 2H).

### Example A346. Synthesis of (3aS,6S,9S,15S,33aS)-22-(5-(4-cyclopropylpiperazin-1-yl)-2-((S)-1-meth oxyethyl)pyridin-3-yl)-23-ethyl-1-(2-fluoro-2-methylpropanoyl)-6-isopropyl-5,19,19-trimethyl-3,3a,5,6,8,9,14,15,19,20,33,33a-dodecahydro-1H,12H,18H-11,15-epimino-24,26-etheno-9,29-methano-27,31-(metheno)dipyrrolo[1,2-e:2′,3-p][1,14]dioxa[5,19,22,25]tetraazacyclotriacontine-4,7,10,16(2H,13H)-tetraone

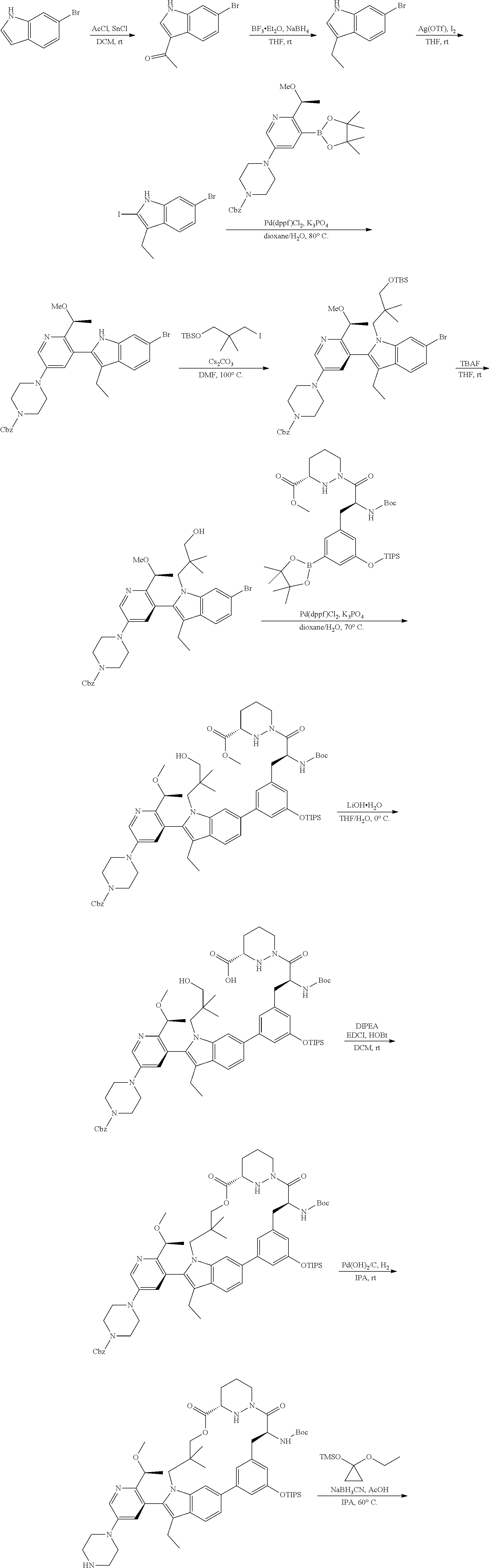

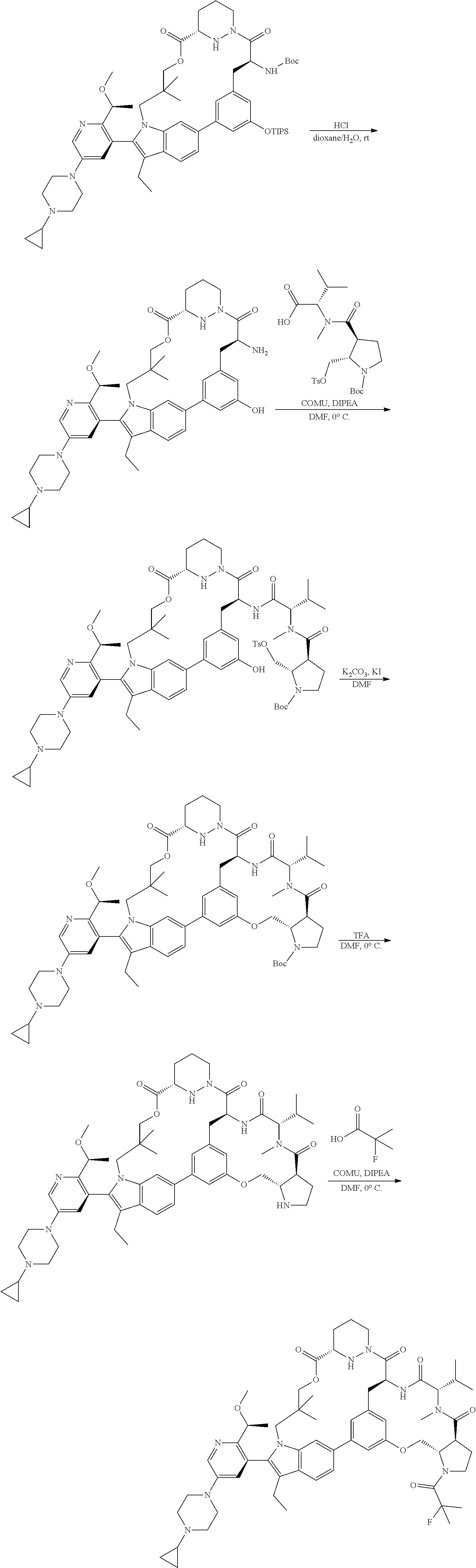

Step 1. To a solution of 6-bromo-1H-indole (20.0 g, 102 mmol) in DCM (400 mL) at 0° C. were added acetyl chloride (16.0 g, 204 mmol) and SnCl₄ (39.9 g, 153 mmol). The resulting mixture was stirred overnight at room temperature and was then quenched at 0° C. by the addition of H₂O. The aqueous layer was extracted with DCM and the combined organic extracts were washed with brine, dried over Na₂SO₄, filtered, and concentrated under reduced pressure. Purification by normal phase chromatography afforded 1-(6-bromo-1H-indol-3-yl)ethan-1-one (20.0 g, 82% yield) as a yellow solid. LCMS (ESI) m/z: [M+H] calcd for C₁₀H₈BrNO: 238.0; found 238.0.

Step 2. To a solution of 1-(6-bromo-1H-indol-3-yl)ethan-1-one (20.0 g, 84.0 mmol) in THE (200 mL) at 0° C. were added NaBH₄ (9.53 g, 252.0 mmol) and BF₃·Et₂O (23.9 g, 168 mmol). The resulting mixture was stirred overnight at room temperature then quenched at 0° C. by the addition of H₂O. The aqueous layer was extracted with EtOAc, and the combined organic extracts were washed with brine, dried over Na₂SO₄, filtered, and concentrated under reduced pressure. Purification by normal phase chromatography (50% EtOAc/pet. ether) afforded 6-bromo-3-ethyl-1H-indole (10 g, 53% yield) as a yellow solid. LCMS (ESI) m/z: [M−H] calcd for C₁₀H₁₀BrN: 222.0; found 222.1.

Step 3. To a solution of 6-bromo-3-ethyl-1H-indole (10.0 g, 44.6 mmol) and silver triflate (13.8 g, 53.6 mmol) in THE (100 mL) at 0° C. was added a solution of iodine (12.5 g, 49.1 mmol) in THE (50 mL). The resulting mixture was stirred for 30 min and was then quenched at 0° C. by the addition of H₂O. The aqueous mixture was extracted with EtOAc, and the combined organic extracts were washed with brine, dried over Na₂SO₄, filtered, and concentrated under reduced pressure. Purification by normal phase chromatography afforded 6-bromo-3-ethyl-2-iodo-1H-indole (9.10 g, 58% yield) as a yellow solid. LCMS (ESI) m/z: [M−H] calcd for C₁₀H₉BrIN: 347.9; found 348.0.

Step 4. To a solution of 6-bromo-3-ethyl-2-iodo-1H-indole (5.0 g, 14.3 mmol), benzyl (S)-4-(6-(1-methoxyethyl)-5-(4,4,5,5-tetramethyl-1,3,2-dioxaborolan-2-yl)pyridin-3-yl)piperazine-1-carboxylate (7.56 g, 15.7 mmol) and K₃PO₄ (6.06 g, 28.6 mmol) in dioxane (48 mL) and H₂O (6.0 mL) at room temperature was added Pd(dppf)Cl₂ (1.05 g, 1.43 mmol). The resulting mixture was stirred for 2 h at 80° C. and was then quenched at room temperature by the addition of H₂O. The aqueous mixture was extracted with EtOAc, and the combined organic extracts were washed with brine, dried over Na₂SO₄, filtered, and concentrated under reduced pressure. Purification by normal phase chromatography afforded benzyl (S)-4-(5-(6-bromo-3-ethyl-1H-indol-2-yl)-6-(1-methoxyethyl)pyridin-3-yl)piperazine-1-carboxylate (7.0 g, 85% yield) as a yellow solid. LCMS (ESI) m/z: [M+H] calcd for C₃₀H₃₃BrN₄O₃: 577.2; found 577.1.

Step 5. To a solution of benzyl (S)-4-(5-(6-bromo-3-ethyl-1H-indol-2-yl)-6-(1-methoxyethyl)pyridin-3-yl)piperazine-1-carboxylate (7.0 g, 12.1 mmol) and Cs₂CO₃ (11.9 g, 36.4 mmol) in THE (70 mL) at 0° C. was added tert-butyl(3-iodo-2,2-dimethylpropoxy)dimethylsilane (15.9 g, 48.5 mmol). The resulting mixture was stirred for 4 h at 100° C. and was then quenched at room temperature by the addition of H₂O. The aqueous mixture was extracted with EtOAc, and the combined organic extracts were washed with brine, dried over Na₂SO₄, filtered, and concentrated under reduced pressure. Purification by reversed phase chromatography afforded benzyl (S)-4-(5-(6-bromo-1-(3-((tert-butyldimethylsilyl)oxy)-2,2-dimethylpropyl)-3-ethyl-1H-indol-2-yl)-6-(1-methoxyethyl)pyridin-3-yl)piperazine-1-carboxylate (3.0 g, 32% yield) as a yellow solid. LCMS (ESI) m/z: [M+H] calcd for C₄₁H₅₇BrN₄O₄Si: 777.3; found 777.4.

Step 6. To a solution of benzyl (S)-4-(5-(6-bromo-1-(3-((tert-butyldimethylsilyl)oxy)-2,2-dimethylpropyl)-3-ethyl-1H-indol-2-yl)-6-(1-methoxyethyl)pyridin-3-yl)piperazine-1-carboxylate (3.0 g, 3.85 mmol) in THE (30 mL) at 0° C. was added TBAF (2.16 g, 7.70 mmol). The resulting mixture was stirred for 3 h at room temperature and was then quenched by the addition of H₂O. The aqueous mixture was extracted with EtOAc, and the combined organic extracts were washed with brine, dried over Na₂SO₄, filtered, and concentrated under reduced pressure. Purification and atropisomer separation by normal phase column chromatography afforded the desired P-atropisomer benzyl (S)-4-(5-(6-bromo-3-ethyl-1-(3-hydroxy-2,2-dimethylpropyl)-1H-indol-2-yl)-6-(1-methoxyethyl)pyridin-3-yl)piperazine-1-carboxylate (1.2 g, 47% yield) and corresponding undesired M-atropisomer (0.8 g, 38% yield). The P-atropisomer was carried on to the subsequent reaction. LCMS (ESI) m/z: [M+H] calcd for C₃₅H₄₃BrN₄O₄: 663.3; found 663.3.

Step 7. To a solution of benzyl (S)-4-(5-(6-bromo-3-ethyl-1-(3-hydroxy-2,2-dimethylpropyl)-1H-indol-2-yl)-6-(1-methoxyethyl)pyridin-3-yl)piperazine-1-carboxylate (1.2 g, 1.81 mmol), methyl (S)-1-((S)-2-((tert-butoxycarbonyl)amino)-3-(3-(4,4,5,5-tetramethyl-1,3,2-dioxaborolan-2-yl)-5-((triisopropylsilyl)oxy)phenyl)propanoyl)hexahydropyridazine-3-carboxylate (1.37 g, 1.99 mmol), and K₃PO₄ (770 mg, 3.62 mmol) in dioxane (10 mL) and H₂O (2.0 mL) at room temperature was added Pd(dppf)Cl₂ (130 mg, 0.181 mmol). The resulting mixture was stirred for 2 h at 70° C. and was then quenched at room temperature by the addition of H₂O. The aqueous mixture was extracted with EtOAc, and the combined organic extracts were washed with brine, dried over Na₂SO₄, filtered, and concentrated under reduced pressure. Purification by normal phase chromatography afforded methyl (S)-1-((S)-3-(3-(2-(5-(4-((benzyloxy)carbonyl)piperazin-1-yl)-2-((S)-1-methoxyethyl)pyridin-3-yl)-3-ethyl-1-(3-hydroxy-2,2-dimethylpropyl)-1H-indol-6-yl)-5-((triisopropylsilyl)oxy)phenyl)-2-((tert-butoxycarbonyl)amino)propanoyl)hexahydropyridazine-3-carboxylate (1.3 g, 63% yield) as a yellow solid. LCMS (ESI) m/z: [M+H] calcd for C₆₄H₉₁N₇O₁₀Si: 1146.7; found 1146.8.

Step 8. To a solution of methyl (S)-1-((S)-3-(3-(2-(5-(4-((benzyloxy)carbonyl)piperazin-1-yl)-2-((S)-1-methoxyethyl)pyridin-3-yl)-3-ethyl-1-(3-hydroxy-2,2-dimethylpropyl)-1H-indol-6-yl)-5-((triisopropylsilyl)oxy)phenyl)-2-((tert-butoxycarbonyl)amino)propanoyl)hexahydropyridazine-3-carboxylate (1.3 g, 1.13 mmol) in THE (10 mL) at 0° C. was added a solution of LiOH·H₂O (238 mg, 5.67 mmol) in H₂O (2.0 mL). The resulting mixture was stirred for 2 h at 0° C. and was then acidified to pH=6 with 1 M aq. HCl. The aqueous mixture was extracted with EtOAc and the combined organic extracts were washed with brine, dried over Na₂SO₄, filtered, and concentrated under reduced pressure to afford (S)-1-((S)-3-(3-(2-(5-(4-((benzyloxy)carbonyl)piperazin-1-yl)-2-((S)-1-methoxyethyl)pyridin-3-yl)-3-ethyl-1-(3-hydroxy-2,2-dimethylpropyl)-1H-indol-6-yl)-5-((triisopropylsilyl)oxy)phenyl)-2-((tert-butoxycarbonyl)amino)propanoyl)hexahydropyridazine-3-carboxylic acid (1.10 g, crude) which was taken directly to the next step without further purification. LCMS (ESI) m/z: [M+H] calcd for C₆₃H₈₉N₇O₁₀Si: 1132.6; found 1132.9.

Step 9. To a solution of (S)-1-((S)-3-(3-(2-(5-(4-((benzyloxy)carbonyl)piperazin-1-yl)-2-((S)-1-methoxyethyl)pyridin-3-yl)-3-ethyl-1-(3-hydroxy-2,2-dimethylpropyl)-1H-indol-6-yl)-5-((triisopropylsilyl)oxy)phenyl)-2-((tert-butoxycarbonyl)amino)propanoyl)hexahydropyridazine-3-carboxylic acid (1.10 g, crude), DIPEA (4.39 g, 33.9 mmol) and HOBt (656 mg, 4.86 mmol) in DCM (110 mL) at 0° C. was added EDCI (5.21 g, 27.2 mmol). The resulting mixture was stirred overnight at room temperature and was then quenched by the addition of H₂O. The aqueous layer was extracted with DCM and the combined organic extracts were washed with brine, dried over Na₂SO₄, filtered, and concentrated under reduced pressure. Purification by normal phase chromatography afforded benzyl 4-(5-((6³S,4S)-4-((tert-butoxycarbonyl)amino)-1³-ethyl-10,10-dimethyl-5,7-dioxo-2⁵-((triisopropylsilyl)oxy)-6¹,6²,6³,6⁴,6⁵,6⁶-hexahydro-1¹H-8-oxa-1(6,1)-indola-6(1,3)-pyridazina-2(1,3)-benzenacycloundecaphane-1²-yl)-6-((S)-1-methoxyethyl)pyridin-3-yl)piperazine-1-carboxylate (600 mg, 48% yield over 2 steps) as a yellow solid. LCMS (ESI) m/z: [M+H] calcd for C₆₃H₈₇N₇O₉Si: 1114.6; found 1114.9.

Step 10. To a solution of benzyl 4-(5-((6³S,4S)-4-((tert-butoxycarbonyl)amino)-1³-ethyl-10,10-dimethyl-5,7-dioxo-2⁵-((triisopropylsilyl)oxy)-6¹,6²,6³,6⁴,6⁵,6⁶-hexahydro-1¹H-8-oxa-1(6,1)-indola-6(1,3)-pyridazina-2(1,3)-benzenacycloundecaphane-1²-yl)-6-((S)-1-methoxyethyl)pyridin-3-yl)piperazine-1-carboxylate (600 mg, 0.538 mmol) in IPA (60 mL) at room temperature was added Pd(OH)₂/C (1.0 g). The resulting mixture was stirred for 1 h under an atmosphere of H₂, filtered, and the filter cake washed with 10:1 vol. DCM/MeOH. The filtrate was concentrated under reduced pressure to afford tert-butyl ((6³S,4S)-1³-ethyl-1²-(2-((S)-1-methoxyethyl)-5-(piperazin-1-yl)pyridin-3-yl)-10,10-dimethyl-5,7-dioxo-2⁵-((triisopropylsilyl)oxy)-6¹,6²,6³,6⁴,6⁵,6⁶-hexahydro-1¹H-8-oxa-1(6,1)-indola-6(1,3)-pyridazina-2(1,3)-benzenacycloundecaphane-4-yl)carbamate (520 mg, crude) which was taken directly to the next reaction without further purification. LCMS (ESI) m/z: [M+H] calcd for C₅₅H₈₁N₇O₇Si: 980.6; found 980.8.

Step 11. To a solution of tert-butyl ((6³S,4S)-1³-ethyl-1²-(2-((S)-1-methoxyethyl)-5-(piperazin-1-yl)pyridin-3-yl)-10,10-dimethyl-5,7-dioxo-2⁵-((triisopropylsilyl)oxy)-6¹,6²,6³,6⁴,6⁵,6⁶-hexahydro-1 ¹H-8-oxa-1(6,1)-indola-6(1,3)-pyridazina-2(1,3)-benzenacycloundecaphane-4-yl)carbamate (520 mg, crude), (1-ethoxycyclopropoxy)trimethylsilane (462 mg, 2.65 mmol), and acetic acid (319 mg, 5.30 mmol) in IPA (6.0 mL) was added NaBH₃CN (50 mg, 0.80 mmol). The resulting mixture was stirred overnight at 60° C. and was then neutralized at room temperature with sat. aq. NaHCO₃. The aqueous mixture was extracted with EtOAc, and the combined organic extracts were washed with brine, dried over Na₂SO₄, filtered, and concentrated under reduced pressure. Purification by normal phase prep-TLC afforded tert-butyl ((6³S,4S)-1²-(5-(4-cyclopropylpiperazin-1-yl)-2-((S)-1-methoxyethyl)pyridin-3-yl)-1³-ethyl-10,10-dimethyl-5,7-dioxo-25-((triisopropylsilyl)oxy)-6¹,6²,6³,6⁴,6⁵,6⁶-hexahydro-1¹H-8-oxa-1(6,1)-indola-6(1,3)-pyridazina-2(1,3)-benzenacycloundecaphane-4-yl)carbamate (200 mg, 36% yield over 2 steps) as a yellow solid. LCMS (ESI) m/z: [M+H] calcd for C₅₈H₈₅N₇O₇Si: 1020.6; found 1020.9.

Step 12. To a solution of tert-butyl ((6³S,4S)-1²-(5-(4-cyclopropylpiperazin-1-yl)-2-((S)-1-methoxyethyl)pyridin-3-yl)-1³-ethyl-10,10-dimethyl-5,7-dioxo-25-((triisopropylsilyl)oxy)-6¹,6²,6³,6⁴,6⁵,6⁶-hexahydro-1¹H-8-oxa-1(6,1)-indola-6(1,3)-pyridazina-2(1,3)-benzenacycloundecaphane-4-yl)carbamate (200 mg, 0.196 mmol) in dioxane (2.0 mL) and H₂O (400 μL) at 0° C. was added 4 M HCl in dioxane (4.0 mL). The resulting mixture was stirred for 3 h at room temperature and was then basified to pH=8 by the addition of sat. aq. NaHCO₃. The resulting aqueous mixture was extracted with EtOAc, and the combined organic extracts were washed with brine, dried over Na₂SO₄, filtered, and concentrated under reduced pressure to afford (6³S,4S)-4-amino-1²-(5-(4-cyclopropylpiperazin-1-yl)-2-((S)-1-methoxyethyl)pyridin-3-yl)-1³-ethyl-2⁵-hydroxy-10,10-dimethyl-6¹,6²,6³,6⁴,6⁵,6⁶-hexahydro-1¹H-8-oxa-1(6,1)-indola-6(1,3)-pyridazina-2(1,3)-benzenacycloundecaphane-5,7-dione (140 mg, crude) which was taken directly to the next reaction without further purification. LCMS (ESI) m/z: [M+H] calcd for C₄₄H₅₇N₇O₇: 764.5; found 764.6.

Step 13. To a solution of (6³S,4S)-4-amino-1²-(5-(4-cyclopropylpiperazin-1-yl)-2-((S)-1-methoxyethyl)pyridin-3-yl)-1³-ethyl-2⁵-hydroxy-10,10-dimethyl-6¹,6²,6³,6⁴,6⁵,6⁶-hexahydro-1 ¹H-8-oxa-1(6,1)-indola-6(1,3)-pyridazina-2(1,3)-benzenacycloundecaphane-5,7-dione (140 mg, crude), (2S)-2-{1-[(2S,3S)-1-(tert-butoxycarbonyl)-2-{[(4-methylbenzenesulfonyl)oxy]methyl}pyrrolidin-3-yl]-N-methylformamido}-3-methylbutanoic acid (140 mg, crude) and DIPEA (947 mg, 7.32 mmol) in DMF (1.4 mL) at 0° C. was added COMU (102 mg, 0.238 mmol). The resulting mixture was stirred for 1 h at 0° C. and was then quenched by the addition of H₂O. The aqueous mixture was extracted with EtOAc, and the combined organic extracts were washed with brine, dried over Na₂SO₄, filtered, and concentrated under reduced pressure. Purification by normal phase prep-TLC afforded tert-butyl (2S,3S)-3-(((2S)-1-(((6³S,4S)-1²-(5-(4-cyclopropylpiperazin-1-yl)-2-((S)-1-methoxyethyl)pyridin-3-yl)-1³-ethyl-2⁵-hydroxy-10,10-dimethyl-5,7-dioxo-6¹,6²,6³,6⁴,6⁵,6⁶-hexahydro-1¹H-8-oxa-1(6,1)-indola-6(1,3)-pyridazina-2(1,3)- benzenacycloundecaphane-4-yl)amino)-3-methyl-1-oxobutan-2-yl)(methyl)carbamoyl)-2-((tosyloxy)methyl)pyrrolidine-1-carboxylate (105 mg, 43% yield over 2 steps) as a yellow solid. LCMS (ESI) m/z: [M+H] calcd for C₆₈H₉₁N₉O₁₂S: 1258.7; found 1258.7.

Step 14. To a solution of tert-butyl (2S,3S)-3-(((2S)-1-(((6³S,4S)-1²-(5-(4-cyclopropylpiperazin-1-yl)-2-((S)-1-methoxyethyl)pyridin-3-yl)-1³-ethyl-2⁵-hydroxy-10,10-dimethyl-5,7-dioxo-6¹,6²,6³,6⁴,6⁵,6⁶-hexahydro-1¹H-8-oxa-1(6,1)-indola-6(1,3)-pyridazina-2(1,3)-benzenacycloundecaphane-4-yl)amino)-3-methyl-1-oxobutan-2-yl)(methyl)carbamoyl)-2-((tosyloxy)methyl)pyrrolidine-1-carboxylate (105 mg, 0.083 mmol), K₂CO₃ (115 mg, 0.830 mmol) in DMF (10 mL) was added KI (13.8 mg, 0.083 mmol). The resulting mixture was stirred for 2 h at 80° C. and was then quenched at room temperature by the addition of H₂O. The aqueous mixture was extracted with EtOAc, and the combined organic extracts were washed with brine, dried over Na₂SO₄, filtered, and concentrated under reduced pressure. Purification by normal phase prep-TLC afforded tert-butyl (3aS,6S,9S,15S,33aS)-22-(5-(4-cyclopropylpiperazin-1-yl)-2-((S)-1-methoxyethyl)pyridin-3-yl)-23-ethyl-6-isopropyl-5,19,19-trimethyl-4,7,10,16-tetraoxo-2,3,3a,4,5,6,7,8,9,10,13,14,15,16,19,20,33,33a-octadecahydro-1H,12H,18H-11,15-epimino-24,26-etheno-9,29-methano-27,31-(metheno)dipyrrolo[1,2-e:2′,3′-p][1,14]dioxa[5,19,22,25]tetraazacyclotriacontine-1-carboxylate (70 mg, 77% yield) as a yellow solid. LCMS (ESI) m/z: [M+H] calcd for C₆₁H₈₃N₉O₉: 1086.6; found 1086.9.

Step 15. To a solution of tert-butyl (3aS,6S,9S,15S,33aS)-22-(5-(4-cyclopropylpiperazin-1-yl)-2-((S)-1-methoxyethyl)pyridin-3-yl)-23-ethyl-6-isopropyl-5,19,19-trimethyl-4,7,10,16-tetraoxo-2,3,3a,4,5,6,7,8,9,10,13,14,15,16,19,20,33,33a-octadecahydro-1H,12H,18H-11,15-epimino-24,26-etheno-9,29-methano-27,31-(metheno)dipyrrolo[1,2-e:2′,3′-p][1,14]dioxa[5,19,22,25]tetraazacyclotriacontine-1-carboxylate (70 mg, 0.064 mmol) in DCM (1.0 mL) at 0° C. was added TFA (300 μL). The resulting mixture was stirred for 1 h at 0° C. and was then neutralized with sat. aq. NaHCO₃. The aqueous layer was extracted with EtOAc, and the combined organic extracts were washed with brine, dried over Na₂SO₄, and concentrated under reduced pressure to afford (3aS,6S,9S,15S,33aS)-22-(5-(4-cyclopropylpiperazin-1-yl)-2-((S)-1-methoxyethyl)pyridin-3-yl)-23-ethyl-6-isopropyl-5,19,19-trimethyl-3,3a,5,6,8,9,14,15,19,20,33,33a-dodecahydro-1H,12H,18H-11,15-epimino-24,26-etheno-9,29-methano-27,31-(metheno)dipyrrolo[1,2-e:2′,3′-p][1,14]dioxa[5,19,22,25]tetraazacyclotriacontine-4,7,10,16(2H,13H)-tetraone (50 mg, crude) which was taken to the next reaction without further purification. LCMS (ESI) m/z: [M+H] calcd for C₅₆H₇₅N₉O₇: 986.6; found 986.7.

Step 16. To a solution of (3aS,6S,9S,15S,33aS)-22-(5-(4-cyclopropylpiperazin-1-yl)-2-((S)-1-methoxyethyl)pyridin-3-yl)-23-ethyl-6-isopropyl-5,19,19-trimethyl-3,3a,5,6,8,9,14,15,19,20,33,33a-dodecahydro-1H,12H,18H-11,15-epimino-24,26-etheno-9,29-methano-27,31-(metheno)dipyrrolo[1,2-e:2′,3′-p][1,14]dioxa[5,19,22,25]tetraazacyclotriacontine-4,7,10,16(2H,13H)-tetraone (40 mg, crude), COMU (22.6 mg, 0.053 mmol) and DIPEA (209 mg, 1.64 mmol) in DMF (1.0 mL) at 0° C. was added 2-fluoro-2-methylpropanoic acid (8.6 mg, 0.082 mmol). The resulting mixture was stirred for 1 h at 0° C. and was then quenched by the addition of H₂O. The aqueous mixture was extracted with EtOAc, and the combined organic extracts were washed with brine, dried over Na₂SO₄, filtered, and concentrated under reduced pressure. Purification by reversed phase chromatography yielded (3aS,6S,9S,15S,33aS)-22-(5-(4-cyclopropylpiperazin-1-yl)-2-((S)-1-methoxyethyl)pyridin-3-yl)-23-ethyl-1-(2-fluoro-2-methylpropanoyl)-6-isopropyl-5,19,19-trimethyl-3,3a,5,6,8,9,14,15,19,20,33,33a-dodecahydro-1H,12H,18H-11,15-epimino-24,26-etheno-9,29-methano-27,31-(metheno)dipyrrolo[1,2-e:2′,3′-p][1,14]dioxa[5,19,22,25]tetraazacyclotriacontine-4,7,10,16(2H,13H)-tetraone (4.9 mg, 7.1% yield over 2 steps) as a white solid. LCMS (ESI) m/z: [M+H] calcd for C₆₀H₈₀FN₉O₈: 1074.6; found 1074.6. ¹H NMR (400 MHz, DMSO-d₆) δ 8.50 (s, 1H), 8.07 (s, 1H), 7.90 (s, 1H), 7.71-7.55 (m, 3H), 7.40-7.18 (m, 3H), 6.84-6.15 (m, 2H), 5.73 (br s, 1H) 5.20-4.99 (m, 2H), 4.72-4.61 (m, 2H), 4.52-4.16 (m, 4H), 4.12-3.88 (m, 4H), 3.78-3.42 (m, 6H), 3.28-3.14 (m, 4H), 3.01-2.55 (m, 8H), 2.33-2.13 (m, 3H), 2.09-1.82 (m, 3H), 1.74-1.37 (m, 11H), 1.24 (t, J=8.4 Hz, 3H), 1.13 (t, J=9.6 Hz, 2H), 0.98-0.33 (m, 16H).

### Example A351. Synthesis of (3aS,6S,9S,15S,32aS)-21-(5-(1-cyclopropyl-4-methylpiperidin-4-yl)-2-((S)-1-methoxyethyl)pyridin-3-yl)-22-ethyl-1-(2-fluoro-2-methylpropanoyl)-6-isopropyl-5,19,19-trimethyl-3,3a,5,6,8,9,14,15,18,19,20,22,32,32a-tetradecahydro-1H,12H-11,15-epimino-23,25-etheno-9,28-methano-26,30-(metheno)dipyrrolo[2,3-c:3′,4-v][1,18]dioxa[6,9,12]triazacyclotriacontine-4,7,10,16(2H,13H)-tetraone

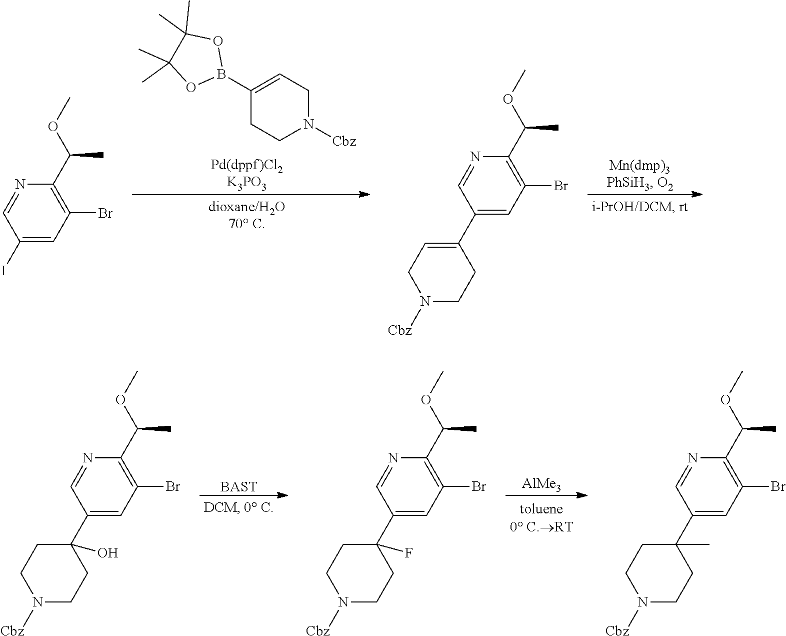

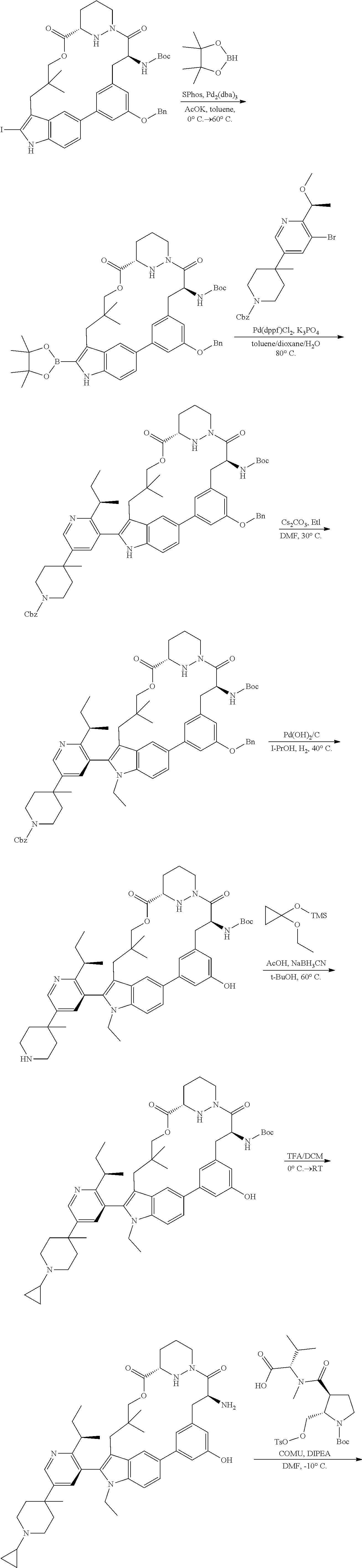

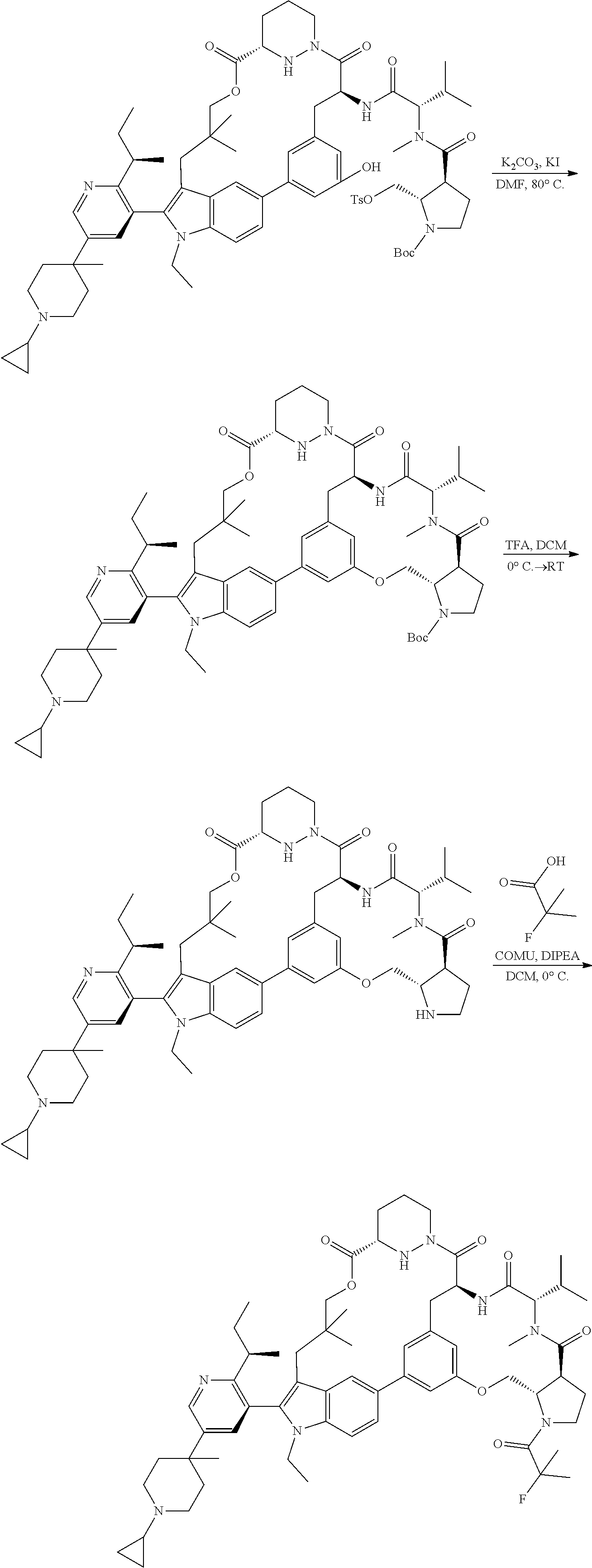

Step 1. To a stirred solution of (S)-3-bromo-5-iodo-2-(1-methoxyethyl)pyridine (17.0 g, 49.7 mmol) and benzyl 4-(4,4,5,5-tetramethyl-1,3,2-dioxaborolan-2-yl)-3,6-dihydropyridine-1 (2-F)-carboxylate (18.8 g, 54.7 mmol) in dioxane (255 mL) was added K₃PO₄ (21.1 g, 99.4 mmol) in H₂O (51 mL) followed by Pd(dppf)Cl₂ (3.64 g, 4.97 mmol). The resulting mixture was heated to 70° C. After stirring for 2 h, the reaction mixture was cooled to room temperature, diluted with EtOAc, washed with brine, dried over Na₂SO₄, filtered, concentrated under reduced pressure, and purified by silica gel column chromatography to afford benzyl (S)-5-bromo-6-(1-methoxyethyl)-3′,6′-dihydro-[3,4′-bipyridine]-1′(2′H)-carboxylate (19.9 g, 93% yield) as a yellow oil. LCMS (ESI) m/z: [M+H] calcd for C₂₁H₂₃BrN₂O₃: 431.1; found 431.0.

Step 2. Under an atmosphere of 02, to a solution of benzyl (S)-5-bromo-6-(1-methoxyethyl)-3′,6′-dihydro-[3,4′-bipyridine]-1′(2′H)-carboxylate (18.0 g, 41.7 mmol) in i-PrOH (315 mL) and DCM (45 mL) was added tris(2,2,6,6-tetramethyl-3,5-heptanedionato)manganese(III) (500 mg, 0.835 mmol) at 0° C. followed by dimethyl(phenyl)silane (11.4 g, 83.5 mmol) dropwise. The mixture was warmed to room temperature and stirred overnight, after which time the mixture was concentrated under reduced pressure and purified by silica gel column chromatography to afford benzyl (S)-4-(5-bromo-6-(1-methoxyethyl)pyridin-3-yl)-4-hydroxypiperidine-1-carboxylate (11.8 g, 63% yield) as a white solid. LCMS (ESI) m/z: [M+H] calcd for C₂₁H₂₅BrN₂O₄: 449.1; found 449.1.

Step 3. To a solution of benzyl (S)-4-(5-bromo-6-(1-methoxyethyl)pyridin-3-yl)-4-hydroxypiperidine-1-carboxylate (10 g, 22.3 mmol) in DCM (200 mL) stirred at 0° C. under an atmosphere of argon was added BAST (6.40 g, 28.9 mmol) in DCM (10 mL) dropwise. After stirring for 1 h at 0° C., the reaction was quenched with the addition of sat. aq. NaHCO₃. The aqueous phase was extracted into DCM, dried over Na₂SO₄, filtered, concentrated under reduced pressure, and purified by silica gel column chromatography to afford benzyl (S)-4-(5-bromo-6-(1-methoxyethyl)pyridin-3-yl)-4-fluoropiperidine-1-carboxylate (7.0 g, 70% yield) as a light yellow oil. LCMS (ESI) m/z: [M+H] calcd for C₂₁H₂₄BrFN₂O₃: 451.1; found 451.1.

Step 4. To a mixture of benzyl (S)-4-(5-bromo-6-(1-methoxyethyl)pyridin-3-yl)-4-fluoropiperidine-1-carboxylate (7.00 g, 15.5 mmol) in toluene (500 mL) stirred at 0° C. under an atmosphere of argon was added AlMe₃ (35 mL, 69.8 mmol, 2 M in toluene) dropwise. The reaction mixture was warmed to room temperature and stirred for 30 min, after which time the reaction was quenched by the addition of ice water, filtered, the filter cake was washed with EtOAc. The filtrate was then concentrated under reduced pressure, and the residue was purified by silica gel column chromatography followed by prep-SFC to afford benzyl (S)-4-(5-bromo-6-(1-methoxyethyl)pyridin-3-yl)-4-methylpiperidine-1-carboxylate (1.2 g, 17% yield) as a yellow oil. LCMS (ESI) m/z: [M+H] calcd for C₂₂H₂₇BrN₂O₃: 447.1; found 447.1.

Step 5. To a solution of tert-butyl ((6³S,4S)-2⁵-(benzyloxy)-1²-iodo-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)carbamate (950 mg, 1.20 mmol) in toluene (10 mL) stirred at 0° C. under an atmosphere of argon were added KOAc (412 mg, 4.20 mmol), SPhos (148 mg, 0.359 mmol), and Pd₂(dba)₃ (132 mg, 0.144 mmol) followed by a solution of 4,4,5,5-tetramethyl-1,3,2-dioxaborolane (1.15 g, 8.99 mmol) in toluene (10 mL) dropwise and the reaction mixture was heated to 60° C. After overnight stirring, the reaction was cooled to 0° C., quenched with sat. aq. NH₄Cl, extracted into EtOAc, washed with brine, dried over Na₂SO₄, filtered, concentrated under reduced pressure, and purified by silica gel column chromatography to afford tert-butyl ((6³S,4S)-2⁵-(benzyloxy) 10,10-dimethyl-5,7-dioxo-1²-(4,4,5,5-tetramethyl-1,3,2-dioxaborolan-2-yl)-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)carbamate (844 mg, 89% yield) as a light yellow oil. LCMS (ESI) m/z: [M+H] calcd for C₄₅H₅₇BN₄O₈: 793.4; found 793.4.

Step 6. To a stirring solution of tert-butyl ((6³S,4S)-2⁵-(benzyloxy)-10,10-dimethyl-5,7-dioxo-1²-(4,4,5,5-tetramethyl-1,3,2-dioxaborolan-2-yl)-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)carbamate (850 mg, 1.07 mmol) and benzyl (S)-4-(5-bromo-6-(1-methoxyethyl)pyridin-3-yl)-4-methylpiperidine-1-carboxylate (528 mg, 1.18 mmol) in dioxane (8 mL) and toluene (8 mL) was added K₃PO₄ (455 mg, 2.14 mmol) in water (4 mL) followed by Pd(dppf)Cl₂ (78.5 mg, 0.107 mmol). The reaction mixture was heated to 80° C. and stirred for 2 h. The mixture was then cooled to room temperature, diluted with EtOAc, washed with H₂O, dried over Na₂SO₄, filtered, and concentrated under reduced pressure to afford benzyl 4-(5-((6³S,4S)-2⁵-(benzyloxy)-4-((tert-butoxycarbonyl)amino)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-1²-yl)-6-((S)-1-methoxyethyl)pyridin-3-yl)-4-methylpiperidine-1-carboxylate (1.15 g, crude) as a yellow solid, which was taken directly to the next reaction without further purification. LCMS (ESI) m/z: [M+H] calcd for C₆₁H₇₂N₆O₉: 1033.6; found 1033.8.

Step 7. To a stirred solution of benzyl 4-(5-((6³S,4S)-2⁵-(benzyloxy)-4-((tert-butoxycarbonyl)amino)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-1²-yl)-6-((S)-1-methoxyethyl)pyridin-3-yl)-4-methylpiperidine-1-carboxylate (1.15 g, crude) and Cs₂CO₃ (1.81 g, 5.565 mmol) in DMF (15 mL) was added iodoethane (0.87 g, 5.565 mmol) dropwise and the reaction mixture was heated to 30° C. After 2 h of stirring, the mixture was diluted with H₂O, extracted into EtOAc, washed with H₂O, dried over Na₂SO₄, filtered, concentrated under reduced pressure, and purified by normal phase prep-TLC to afford benzyl 4-(5-((6³S,4S)-2⁵-(benzyloxy)-4-((tert-butoxycarbonyl)amino)-1¹-ethyl-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-1²-yl)-6-((S)-1-methoxyethyl)pyridin-3-yl)-4-methylpiperidine-1-carboxylate (340 mg, 29% yield over 2 steps) as a yellow solid. LCMS (ESI) m/z: [M+H] calcd for C₆₃H₇₆N₆O₉: 1061.6; found 1061.5.

Step 8. To a stirred solution of benzyl 4-(5-((6³S,4S)-2⁵-(benzyloxy)-4-((tert-butoxycarbonyl)amino)-1¹-ethyl-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-1²-yl)-6-((S)-1-methoxyethyl)pyridin-3-yl)-4-methylpiperidine-1-carboxylate (440 mg, 0.415 mmol) in i-PrOH (100 mL) was added Pd(OH)₂/C (220 mg, 1.57 mmol). The resulting mixture was heated to 40° C. and placed under an atmosphere of H₂. After stirring overnight, the reaction mixture was filtered, the filter cake washed with DCM/i-PrOH (10:1 vol.), and the filtrate concentrated under reduced pressure to afford tert-butyl ((6³S,4S)-1¹-ethyl-2⁵-hydroxy-1²-(2-((S)-1-methoxyethyl)-5-(4-methylpiperidin-4-yl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-1²-yl)-6-((S)-1-methoxyethyl)pyridin-3-yl)-4-methylpiperidine-1-carboxylate (330 mg, crude), which was used directly in the next reaction without further purification. LCMS (ESI) m/z: [M+H] calcd for C₄₈H₆₄N₆O₇: 837.5; found 837.5.

Step 9. To a stirred solution of tert-butyl ((6³S,4S)-1¹-ethyl-2⁵-hydroxy-1²-(2-((S)-1-methoxyethyl)-5-(4-methylpiperidin-4-yl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-1²-yl)-6-((S)-1-methoxyethyl)pyridin-3-yl)-4-methylpiperidine-1-carboxylate (330 mg, crude) in tert-butanol (60 mL) were added NaBH₃CN (49.6 mg, 0.788 mmol), AcOH (237 mg, 3.94 mmol), and (1-ethoxycyclopropoxy)trimethylsilane (344 mg, 1.97 mmol). The reaction mixture was heated to 60° C. and stirred for 2 h. After this time, the mixture was basified to pH 8-9 with sat. aq. NaHCO₃, extracted into EtOAc, washed with brine, dried over Na₂SO₄, filtered, and concentrated under reduced pressure to afford tert-butyl ((6³S,4S)-1²-(5-(1-cyclopropyl-4-methylpiperidin-4-yl)-2-((S)-1-methoxyethyl)pyridin-3-yl)-1¹-ethyl-2⁵-hydroxy-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)carbamate (130 mg, crude) as a light brown solid, which was used in the next step directly without further purification. LCMS (ESI) m/z: [M+H] calcd for C₅₁H₆₈N₆O₇: 877.5; found 877.7.

Step 10. To a solution of tert-butyl ((6³S,4S)-1²-(5-(1-cyclopropyl-4-methylpiperidin-4-yl)-2-((S)-1-methoxyethyl)pyridin-3-yl)-1¹-ethyl-2⁵-hydroxy-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)carbamate (130 mg, crude) in DCM (5 mL) at 0° C. was added TFA (1 mL) dropwise. The reaction mixture was warmed to room temperature and stirred for 2 h. The resulting mixture was then concentrated under reduced pressure, the residue was dissolved in DCM, basified to pH=8 with sat. aq. NaHCO₃, back-extracted into DCM, washed with H₂O, dried over Na₂SO₄, filtered, and concentrated under reduced pressure to afford (6³S,4S)-4-amino-1²-(5-(1-cyclopropyl-4-methylpiperidin-4-yl)-2-((S)-1-methoxyethyl)pyridin-3-yl)-1¹-ethyl-2⁵-hydroxy-10,10-dimethyl-6¹,6²,6³,6⁴,6⁵,6⁶-hexahydro-1¹H-8-oxa-1(5,3)-indola-6(1,3)-pyridazina-2(1,3)- benzenacycloundecaphane-5,7-dione (100 mg, crude) as a light brown solid, which was used in the next step directly without further purification. LCMS (ESI) m/z: [M+H] calcd for C₄₆H₆₀N₆O₅: 777.5; found 777.3.

Step 11. A solution of (6³S,4S)-4-amino-1²-(5-(1-cyclopropyl-4-methylpiperidin-4-yl)-2-((S)-1-methoxyethyl)pyridin-3-yl)-1¹-ethyl-2⁵-hydroxy-10,10-dimethyl-6¹,6²,6³,6⁴,6⁵,6⁶-hexahydro-1 ¹H-8-oxa-1(5,3)-indola-6(1,3)-pyridazina-2(1,3)-benzenacycloundecaphane-5,7-dione (100 mg, crude), N-((2S,3S)-1-(tert-butoxycarbonyl)-2-((tosyloxy)methyl)pyrrolidine-3-carbonyl)-N-methyl-L-valine (85.8 mg, 0.168 mmol), and DIPEA (333 mg, 2.58 mmol) in DMF (5 mL) stirred at 0° C. was cooled to −10° C. and COMU (71.7 mg, 0.168 mmol) was then added in portions. The reaction mixture was stirred for 1 h, after which time the mixture was warmed to 0° C., quenched with H₂O, extracted into DCM, washed with H₂O, dried over Na₂SO₄, filtered, concentrated under reduced pressure, and purified by normal phase prep-TLC to afford tert-butyl (2S,3S)-3-(((2S)-1-(((6³S,4S)-1²-(5-(1-cyclopropyl-4-methylpiperidin-4-yl)-2-((S)-1-methoxyethyl)pyridin-3-yl)-1¹-ethyl-2⁵-hydroxy-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)amino)-3-methyl-1-oxobutan-2-yl)(methyl)carbamoyl)-2-((tosyloxy)methyl)pyrrolidine-1-carboxylate (100 mg, 61% yield over 4 steps) as a light brown solid. LCMS (ESI) m/z: [M+H] calcd for C₇₀H₉₄N₈O₁₂S: 1271.7; found 1272.4.

Step 12. To a stirred solution of tert-butyl (2S,3S)-3-(((2S)-1-(((6³S,4S)-1²-(5-(1-cyclopropyl-4-methylpiperidin-4-yl)-2-((S)-1-methoxyethyl)pyridin-3-yl)-1¹-ethyl-2⁵-hydroxy-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)amino)-3-methyl-1-oxobutan-2-yl)(methyl)carbamoyl)-2-((tosyloxy)methyl)pyrrolidine-1-carboxylate (100 mg, 0.079 mmol) and KI (13.1 mg, 0.079 mmol) in DMF (6 mL) was added K₂CO₃ (109 mg, 0.790 mmol) in portions. The reaction mixture was heated to 80° C. and stirred for 2 h, after which time it was cooled to room temperature, diluted with H₂O, extracted into EtOAc, washed with H₂O, dried over Na₂SO₄, filtered, concentrated under reduced pressure, and purified by normal phase prep-TLC to afford tert-butyl (3aS,6S,9S,15S,32aS)-21-(5-(1-cyclopropyl-4-methylpiperidin-4-yl)-2-((S)-1-methoxyethyl)pyridin-3-yl)-22-ethyl-6-isopropyl-5,19,19-trimethyl-4,7,10,16-tetraoxo-2,3,3a,4,5,6,7,8,9,10,13,14,15,16,18,19,20,22,32,32a-icosahydro-1H,12H-11,15-epimino-23,25-etheno-9,28-methano-26,30-(metheno)dipyrrolo[2,3-c:3′,4′-v][1,18]dioxa[6,9,12]triazacyclotriacontine-1-carboxylate (70 mg, 81% yield) as a light brown solid. LCMS (ESI) m/z: [M+H] calcd for C₆₃H₈₆N₈O₉: 1099.7; found 1099.4.

Step 13. To a solution of tert-butyl (3aS,6S,9S,15S,32aS)-21-(5-(1-cyclopropyl-4-methylpiperidin-4-yl)-2-((S)-1-methoxyethyl)pyridin-3-yl)-22-ethyl-6-isopropyl-5,19,19-trimethyl-4,7,10,16-tetraoxo-2,3,3a,4,5,6,7,8,9,10,13,14,15,16,18,19,20,22,32,32a-icosahydro-1H,12H-11,15-epimino-23,25-etheno-9,28-methano-26,30-(metheno)dipyrrolo[2,3-c:3′,4′-v][1,18]dioxa[6,9,12]triazacyclotriacontine-1-carboxylate (70 mg, 0.064 mmol) in DCM (5 mL) stirred at 0° C. was added TFA (1 mL) dropwise. The reaction mixture was warmed to room temperature and stirred for 1 h, after which time the mixture was concentrated under reduced pressure, diluted with DCM (10 mL), basified to pH=8 with sat. aq. NaHCO₃, extracted into DCM, washed with H₂O, dried over Na₂SO₄, filtered, and concentrated under reduced pressure to afford (3aS,6S,9S,15S,32aS)-21-(5-(1-cyclopropyl-4-methylpiperidin-4-yl)-2-((S)-1-methoxyethyl)pyridin-3-yl)-22-ethyl-6-isopropyl-5,19,19-trimethyl-3,3a,5,6,8,9,14,15,18,19,20,22,32,32a-tetradecahydro-1H,12H-11,15-epimino-23,25-etheno-9,28-methano-26,30-(metheno)dipyrrolo[2,3-c:3′,4′-v][1,18]dioxa[6,9,12]triazacyclotriacontine-4,7,10,16(2H,13H)-tetraone (60 mg, crude) as a light brown solid, which was used in the next reaction without further purification. LCMS (ESI) m/z: [M+H] calcd for C₅₈H₇₈N₈O₇: 999.6; found 999.4.

Step 14. To a suspension of (3aS,6S,9S,15S,32aS)-21-(5-(1-cyclopropyl-4-methylpiperidin-4-yl)-2-((S)-1-methoxyethyl)pyridin-3-yl)-22-ethyl-6-isopropyl-5,19,19-trimethyl-3,3a,5,6,8,9,14,15,18,19,20,22,32,32a-tetradecahydro-1H,12H-11,15-epimino-23,25-etheno-9,28-methano-26,30-(metheno)dipyrrolo[2,3-c:3′,4′-v][1,18]dioxa[6,9,12]triazacyclotriacontine-4,7,10,16(2H,13H)-tetraone (60 mg, crude), 2-fluoro-2-methylpropanoic acid (31.9 mg, 0.300 mmol), and DIPEA (155 mg, 1.20 mmol) in DMF (5 mL) stirred at 0° C. was added COMU (33.4 mg, 0.078 mmol) in portions. The reaction mixture was stirred for 1 h, and was then quenched at 0° C. with H₂O, extracted into DCM, washed with H₂O, dried over Na₂SO₄, filtered, concentrated under reduced pressure, and purified by normal phase prep-TLC followed by reversed phase column chromatography to afford (3aS,6S,9S,15S,32aS)-21-(5-(1-cyclopropyl-4-methylpiperidin-4-yl)-2-((S)-1-methoxyethyl)pyridin-3-yl)-22-ethyl-1-(2-fluoro-2-methylpropanoyl)-6-isopropyl-5,19,19-trimethyl-3,3a,5,6,8,9,14,15,18,19,20,22,32,32a-tetradecahydro-1H,12H-11,15-epimino-23,25-etheno-9,28-methano-26,30-(metheno)dipyrrolo[2,3-c:3′,4′-v][1,18]dioxa[6,9,12]triazacyclotriacontine-4,7,10,16(2H,13H)-tetraone (6.0 mg, 9% yield) as a light yellow solid. LCMS (ESI) m/z: [M+H] calcd for C₆₂H₈₃FN₈O₈: 1087.6; found 1087.5. ¹H NMR (400 MHz, DMSO-d₆) δ 8.77 (dd, J=4.7, 2.3 Hz, 1H), 8.22-7.64 (m, 2H), 7.62-7.08 (m, 4H), 6.83-6.11 (m, 2H), 5.49-4.93 (m, 2H), 4.68 (d, J=10.7 Hz, 1H), 4.54-4.13 (m, 4H), 4.13-3.91 (m, 3H), 3.89-3.56 (m, 3H), 3.58-3.40 (m, 2H), 3.25 (s, 3H), 3.03 (d, J=12.1 Hz, 2H), 2.75 (s, 4H), 2.70-2.58 (m, 1H), 2.38 (s, 3H), 2.26 (dd, J=12.0, 6.2 Hz, 2H), 2.17-1.95 (m, 4H), 1.87-1.79 (m, 1H), 1.78-1.63 (m, 4H), 1.60 (d, J=6.6 Hz, 2H), 1.57-1.51 (m, 3H), 1.49 (s, 2H), 1.45-1.37 (m, 4H), 1.30-1.19 (m, 6H), 1.06-0.90 (m, 3H), 0.85-0.77 (m, 5H), 0.67 (d, J=6.0 Hz, 3H), 0.60 (s, 2H), 0.41 (s, 1H), 0.38 (dd, J=6.5, 2.2 Hz, 2H), 0.26 (q, J=3.3 Hz, 2H).

### Example A354. Synthesis of (3aS,6S,9S,15S,32aS)-22-ethyl-1-(2-fluoro-2-methylpropanoyl)-6-isopropyl-21-(2-((S)-1-methoxyethyl)-5-(pyridin-3-ylmethoxy)pyridin-3-yl)-5,19,19-trimethyl-3,3a,5,6,8,9,14,15,18,19,20,22,32,32a-tetradecahydro-1H,12H-11,15-epimino-23,25-etheno-9,28-methano-26,30-(metheno)dipyrrolo[2,3-c:3′,4′-v][1,18]dioxa[6,9,12]triazacyclotriacontine-4,7,10,16(2H,13H)-tetraone

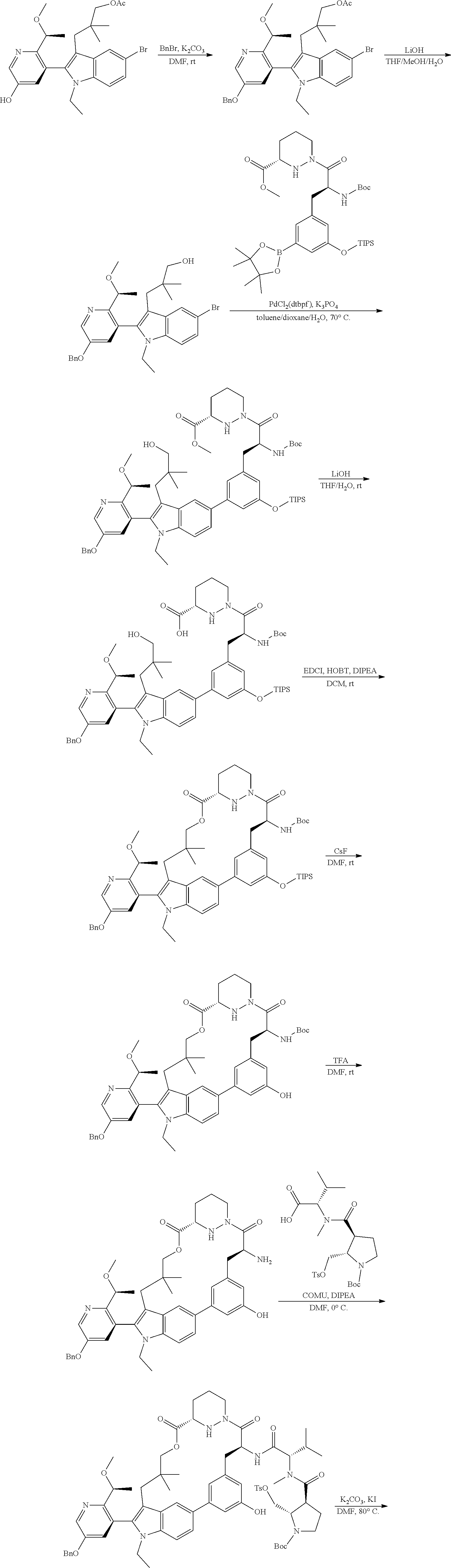

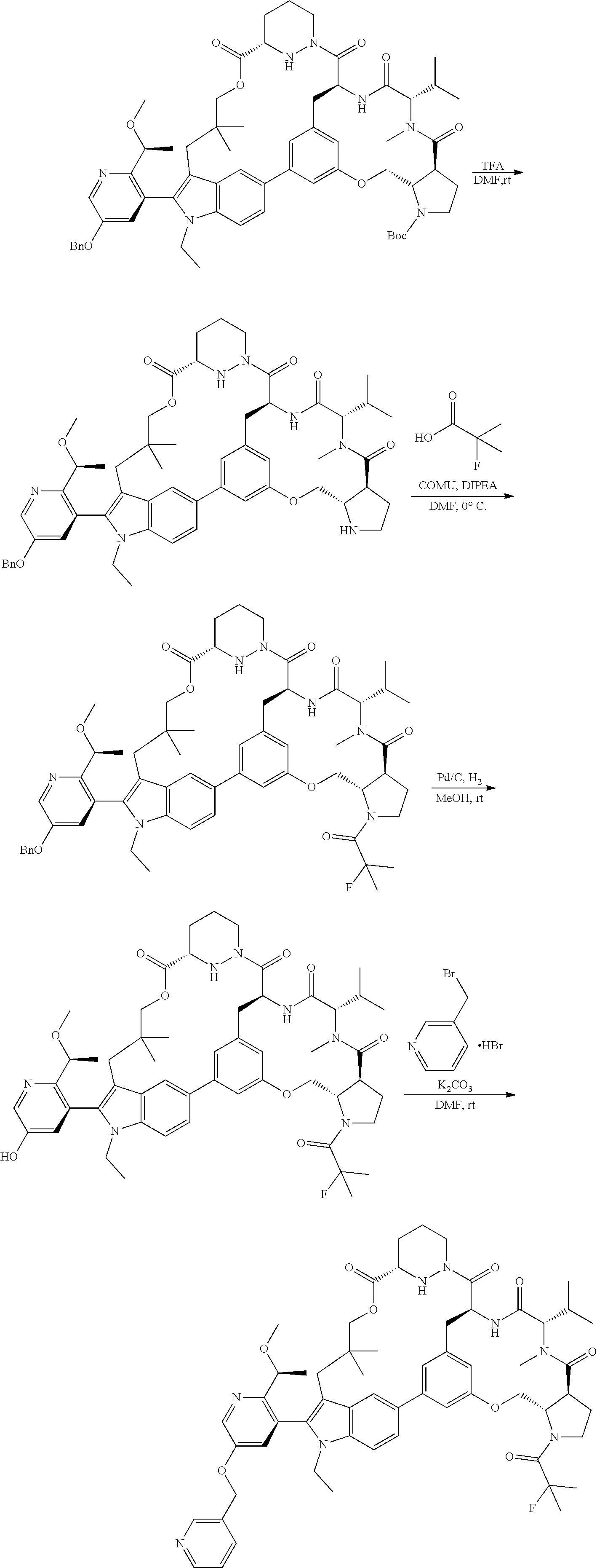

Step 1. To a solution of (S)-3-(5-bromo-1-ethyl-2-(5-hydroxy-2-(1-methoxyethyl)pyridin-3-yl)-1H-indol-3-yl)-2,2-dimethylpropyl acetate (8.40 g, 16.7 mmol) in DMF (80 mL) at 0° C. were added K₂CO₃ (4.61 g, 33.4 mmol) and benzyl bromide (3.14 g, 18.4 mmol). The resulting mixture was stirred for 1.5 h at room temperature and quenched at 0° C. by the addition of cold H₂O. The aqueous mixture was extracted with EtOAc (3×100 mL), and the combined organic extracts were washed with brine (3×80 mL), dried over Na₂SO₄, filtered, and concentrated under reduced pressure. Purification by normal phase chromatography afforded (S)-3-(2-(5-(benzyloxy)-2-(1-methoxyethyl)pyridin-3-yl)-5-bromo-1-ethyl-1H-indol-3-yl)-2,2-dimethylpropyl acetate (8.84 g, 80% yield) as a yellow solid. LCMS (ESI) m/z: [M+H] calcd for C₃₂H₃₇BrN₂O₄: 593.2; found 593.3.

Step 2. To a solution of (S)-3-(2-(5-(benzyloxy)-2-(1-methoxyethyl)pyridin-3-yl)-5-bromo-1-ethyl-1H-indol-3-yl)-2,2-dimethylpropyl acetate (8.74 g, 14.7 mmol) in THE (53 mL) and MeOH (18 mL) at 0° C. was added a solution of LiOH·H₂O (1.24 g, 29.5 mmol) in H₂O (18 mL). The resulting mixture was stirred for 2 h at room temperature and concentrated under reduced pressure. The concentrate was extracted with EtOAc (3×50 mL), and the combined organic extracts were dried over Na₂SO₄, filtered, and concentrated under reduced pressure to afford (S)-3-(2-(5-(benzyloxy)-2-(1-methoxyethyl)pyridin-3-yl)-5-bromo-1-ethyl-1H-indol-3-yl)-2,2-dimethylpropan-1-ol (7.69 g, crude) which was taken directly to the next reaction without further purification. LCMS (ESI) m/z: [M+H] calcd for C₃₀H₃₅BrN₂O₃: 551.19; found 551.3.

Step 3. To a solution of (S)-3-(2-(5-(benzyloxy)-2-(1-methoxyethyl)pyridin-3-yl)-5-bromo-1-ethyl-1H-indol-3-yl)-2,2-dimethylpropan-1-ol (7.69 g, crude) and methyl (S)-1-((S)-2-((tert-butoxycarbonyl)amino)-3-(3-(4,4,5,5-tetramethyl-1,3,2-dioxaborolan-2-yl)-5-((triisopropylsilyl)oxy)phenyl)propanoyl)hexahydropyridazine-3-carboxylate (11.5 g, 16.7 mmol) in toluene (60 mL), dioxane (20 mL) and H₂O (20 mL) were added K₃PO₄ (5.92 g, 27.9 mmol) and PdCl₂(dtbpf) (910 mg, 1.39 mmol). The resulting mixture was stirred for 2 h at 70° C. and concentrated under reduced pressure. The concentrate was filtered, the filter cake washed with EtOAc (3×100 mL), and the filtrate concentrated under reduced pressure to afford methyl (S)-1-((S)-3-(3-(2-(5-(benzyloxy)-2-((S)-1-methoxyethyl)pyridin-3-yl)-1-ethyl-3-(3-hydroxy-2,2-dimethylpropyl)-1H-indol-5-yl)-5-((triisopropylsilyl)oxy)phenyl)-2-((tert-butoxycarbonyl)amino)propanoyl)hexahydropyridazine-3-carboxylate (18.1 g, crude) which was taken to the next reaction without further purification. LCMS (ESI) m/z: [M+H] calcd for C₅₉H₈₃N₅O₉Si: 1034.6; found 1034.6.

Step 4. To a solution of methyl (S)-1-((S)-3-(3-(2-(5-(benzyloxy)-2-((S)-1-methoxyethyl)pyridin-3-yl)-1-ethyl-3-(3-hydroxy-2,2-dimethylpropyl)-1H-indol-5-yl)-5-((triisopropylsilyl)oxy)phenyl)-2-((tert-butoxycarbonyl)amino)propanoyl)hexahydropyridazine-3-carboxylate (18.1 g, crude) in THE (90 mL) stirred at 0° C. was added a solution of LiOH·H₂O (1.10 g, 26.2 mmol) in H₂O (90 mL). The resulting mixture was stirred for 2 h at room temperature, acidified to pH=6 by the addition of 2 M aq. HCl, and concentrated under reduced pressure. The concentrate was extracted with EtOAc (3×200 mL), and the combined organic extracts were dried over Na₂SO₄, filtered, and concentrated under reduced pressure to afford (S)-1-((S)-3-(3-(2-(5-(benzyloxy)-2-((S)-1-methoxyethyl)pyridin-3-yl)-1-ethyl-3-(3-hydroxy-2,2-dimethylpropyl)-1H-indol-5-yl)-5-((triisopropylsilyl)oxy)phenyl)-2-((tert-butoxycarbonyl)amino)propanoyl)hexahydropyridazine-3-carboxylic acid (18 g, crude) which was taken to the next reaction without further purification. LCMS (ESI) m/z: [M+H] calcd for C₅₈H₈₁N₅O₉Si: 1020.6; found 1020.7.

Step 5. To a solution of (S)-1-((S)-3-(3-(2-(5-(benzyloxy)-2-((S)-1-methoxyethyl)pyridin-3-yl)-1-ethyl-3-(3-hydroxy-2,2-dimethylpropyl)-1H-indol-5-yl)-5-((triisopropylsilyl)oxy)phenyl)-2-((tert-butoxycarbonyl)amino)propanoyl)hexahydropyridazine-3-carboxylic acid (17.8 g, crude) and DIPEA (91.2 mL, 523 mmol) in DCM (1.78 L) at 0° C. were added EDCI (66.9 g, 348 mmol) and HOBT (23.6 g, 174 mmol). The resulting mixture was stirred overnight at room temperature, concentrated under reduced pressure, and the concentrate neutralized by the addition of 2 M aq. HCl. The aqueous mixture was extracted with DCM (3×400 mL), and the combined organic extracts were dried over Na₂SO₄, filtered, and concentrated under reduced pressure. Purification by normal phase chromatography afforded tert-butyl ((6³S,4S)-1²-(5-(benzyloxy)-2-((S)-1-methoxyethyl)pyridin-3-yl)-1¹-ethyl-10,10-dimethyl-5,7-dioxo-2⁵-((triisopropylsilyl)oxy)-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)carbamate (9.80 g, 67% yield over 4 steps) as a yellow solid. LCMS (ESI) m/z: [M+H] calcd for C₅₈H₇₉N₅O₈Si: 1002.6; found 1002.7.

Step 6. To a solution of tert-butyl ((6³S,4S)-1²-(5-(benzyloxy)-2-((S)-1-methoxyethyl)pyridin-3-yl)-1¹-ethyl-10,10-dimethyl-5,7-dioxo-2⁵-((triisopropylsilyl)oxy)-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)carbamate (9.70 g, 9.70 mmol) in DMF (90 mL) at 0° C. was added CsF (14.7 g, 96.8 mmol). The resulting mixture was stirred for 2 h at room temperature and quenched at 0° C. by the addition of cold H₂O. The aqueous mixture was extracted with EtOAc (3×150 mL), and the combined organic extracts were washed with brine (3×100 mL), dried over Na₂SO₄, filtered, and concentrated under reduced pressure. Purification by normal phase chromatography afforded tert-butyl ((6³S,4S)-1²-(5-(benzyloxy)-2-((S)-1-methoxyethyl)pyridin-3-yl)-1¹-ethyl-2⁵-hydroxy-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)carbamate (7.00 g, 77% yield) as a white solid. LCMS (ESI) m/z: [M+H] calcd for C₄₉H₅₉N₅O₈: 846.5; found 846.4.

Step 7. To a solution of tert-butyl ((6³S,4S)-1²-(5-(benzyloxy)-2-((S)-1-methoxyethyl)pyridin-3-yl)-1¹-ethyl-2⁵-hydroxy-10,10-dimethyl-5,7-dioxo-6¹,6²,6³,6⁴,6⁵,6⁶-hexahydro-1¹H-8-oxa-1(5,3)-indola-6(1,3)-pyridazina- 2(1,3)-benzenacycloundecaphane-4-yl)carbamate (6.90 g, 8.16 mmol) in DCM (54 mL) at 0° C. was added TFA (18 mL). The resulting mixture was stirred for 1 h at room temperature, concentrated under reduced pressure, and basified to pH=8 by the addition of sat. aq. NaHCO₃. The aqueous mixture was extracted with DCM (3×50 mL), and the combined organic extracts were dried over Na₂SO₄, filtered, and concentrated under reduced pressure to afford (6³S,4S)-4-amino-1²-(5-(benzyloxy)-2-((S)-1-methoxyethyl)pyridin-3-yl)-1¹-ethyl-2⁵-hydroxy-10,10-dimethyl-6¹,6²,6³,6⁴,6⁵,6⁶-hexahydro-1 ¹H-8-oxa-1(5,3)-indola-6(1,3)-pyridazina-2(1,3)-benzenacycloundecaphane-5,7-dione (5.40 g, crude) which was taken to the next reaction without further purification. LCMS (ESI) m/z: [M+H] calcd for C₄₄H₅₁N₅O₆: 746.4; found 746.4.

Step 8. To a solution of (6³S,4S)-4-amino-1²-(5-(benzyloxy)-2-((S)-1-methoxyethyl)pyridin-3-yl)-1¹-ethyl-2⁵-hydroxy-10,10-dimethyl-6¹,6²,6³,6⁴,6⁵,6⁶-hexahydro-1¹H-8-oxa-1(5,3)-indola-6(1,3)-pyridazina-2(1,3)-benzenacycloundecaphane-5,7-dione (5.40 g, crude) and DIPEA (50 mL, 289 mmol) in DMF (50 mL) at 0° C. were added N-((2S,3S)-1-(tert-butoxycarbonyl)-2-((tosyloxy)methyl)pyrrolidine-3-carbonyl)-N-methyl-L-valine (4.82 g, 9.41 mmol) and COMU (4.03 g, 9.41 mmol). The resulting mixture was stirred for 1 h at 0° C. and quenched at 0° C. by the addition of cold H₂O. The aqueous mixture was extracted with EtOAc (3×50 mL), and the combined organic extracts were washed with brine (3×100 mL), dried over Na₂SO₄, filtered, and concentrated under reduced pressure. Purification by normal phase chromatography afforded tert-butyl (2S,3S)-3-(((2S)-1-(((6³S,4S)-1²-(5-(benzyloxy)-2-((S)-1-methoxyethyl)pyridin-3-yl)-1¹-ethyl-2⁵-hydroxy-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)amino)-3-methyl-1-oxobutan-2-yl)(methyl)carbamoyl)-2-((tosyloxy)methyl)pyrrolidine-1-carboxylate (7.26 g, 72% yield over 2 steps) as an off-white solid. LCMS (ESI) m/z: [M+H] calcd for C₆₈H₈₅N₇O₁₃S: 1240.6; found 1240.6.

Step 9. To a solution of tert-butyl (2S,3S)-3-(((2S)-1-(((6³S,4S)-1²-(5-(benzyloxy)-2-((S)-1-methoxyethyl)pyridin-3-yl)-1¹-ethyl-2⁵-hydroxy-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)amino)-3-methyl-1-oxobutan-2-yl)(methyl)carbamoyl)-2-((tosyloxy)methyl)pyrrolidine-1-carboxylate (7.16 g, 5.77 mmol) in DMF (716 mL) at room temperature were added K₂CO₃ (7.98 g, 57.7 mmol) and KI (958 mg, 5.77 mmol). The resulting mixture was stirred for 2 h at 80° C. and quenched at 0° C. by the addition of cold H₂O. The aqueous mixture was extracted with EtOAc (3×700 mL), and the combined organic extracts were washed with brine (3×500 mL), dried over Na₂SO₄, filtered, and concentrated under reduced pressure. Purification by normal phase chromatography afforded tert-butyl (3aS,6S,9S,15S,32aS)-21-(5-(benzyloxy)-2-((S)-1-methoxyethyl)pyridin-3-yl)-22-ethyl-6-isopropyl-5,19,19-trimethyl-4,7,10,16-tetraoxo-2,3,3a,4,5,6,7,8,9,10,13,14,15,16,18,19,20,22,32,32a-icosahydro-1H,12H-11,15-epimino-23,25-etheno-9,28-methano-26,30-(metheno)dipyrrolo[2,3-c:3′,4′-v][1,18]dioxa[6,9,12]triazacyclotriacontine-1-carboxylate (5.28 g, 81% yield) as a white solid. LCMS (ESI) m/z: [M+NH₄] calcd for C₆₁H₇₇N₇O₁₀: 1085.6; found 1085.6.

Step 10. To a solution of tert-butyl (3aS,6S,9S,15S,32aS)-21-(5-(benzyloxy)-2-((S)-1-methoxyethyl)pyridin-3-yl)-22-ethyl-6-isopropyl-5,19,19-trimethyl-4,7,10,16-tetraoxo-2,3,3a,4,5,6,7,8,9,10,13,14,15,16,18,19,20,22,32,32a-icosahydro-1H,12H-11,15-epimino-23,25-etheno-9,28-methano-26,30-(metheno)dipyrrolo[2,3-c:3′,4′-v][1,18]dioxa[6,9,12]triazacyclotriacontine-1-carboxylate (280 mg, 0.262 mmol) in DCM (10 mL) at 0° C. was added TFA (2.0 mL). The resulting mixture was stirred for 1 h at room temperature, concentrated under reduced pressure, and the concentrate basified to pH=8 by the addition of sat. aq. NaHCO₃. The aqueous mixture was extracted with DCM (3×10 mL), and the combined organic extracts were washed with brine (3×10 mL), dried over Na₂SO₄, filtered, and concentrated under reduced pressure to afford (3aS,6S,9S,15S,32aS)-21-(5-(benzyloxy)-2-((S)-1-methoxyethyl)pyridin-3-yl)-22-ethyl-6-isopropyl-5,19,19-trimethyl-3,3a,5,6,8,9,14,15,18,19,20,22,32,32a-tetradecahydro-1H,12H-11,15-epimino-23,25-etheno-9,28-methano-26,30-(metheno)dipyrrolo[2,3-c:3′,4′-v][1,18]dioxa[6,9,12]triazacyclotriacontine-4,7,10,16(2H,13H)-tetraone (251 mg, crude) which was taken to the next reaction without further purification. LCMS (ESI) m/z: [M+H] calcd for C₅₆H₆₉N₇O₈: 968.5; found 968.6.

Step 11. To a solution of (3aS,6S,9S,15S,32aS)-21-(5-(benzyloxy)-2-((S)-1-methoxyethyl)pyridin-3-yl)-22-ethyl-6-isopropyl-5,19,19-trimethyl-3,3a,5,6,8,9,14,15,18,19,20,22,32,32a-tetradecahydro-1H,12H-11,15-epimino-23,25-etheno-9,28-methano-26,30-(metheno)dipyrrolo[2,3-c:3′,4′-v][1,18]dioxa[6,9,12]triazacyclotriacontine-4,7,10,16(2H,13H)-tetraone (251 mg, crude) and DIPEA (450 mL, 2.59 mmol) in DMF (2.0 mL) at 0° C. were added 2-fluoro-2-methylpropanoic acid (55.0 mg, 0.518 mmol) and COMU (166 mg, 0.389 mmol). The resulting mixture was stirred for 30 min at 0° C. and was then quenched by the addition of cold H₂O. The aqueous mixture was extracted with EtOAc (3×20 mL), and the combined organic extracts were washed with brine (3×10 mL), dried over Na₂SO₄, filtered, and concentrated under reduced pressure. Purification by normal phase chromatography afforded (3aS,6S,9S,15S,32aS)-21-(5-(benzyloxy)-2-((S)-1-methoxyethyl)pyridin-3-yl)-22-ethyl-1-(2-fluoro-2-methylpropanoyl)-6-isopropyl-5,19,19-trimethyl-3,3a,5,6,8,9,14,15,18,19,20,22,32,32a-tetradecahydro-1H,12H-11,15-epimino-23,25-etheno-9,28-methano-26,30-(metheno)dipyrrolo[2,3-c:3′,4′-v][1,18]dioxa[6,9,12]triazacyclotriacontine-4,7,10,16(2H,13H)-tetraone (240 mg, 87% yield over 2 steps) as a light yellow solid. LCMS (ESI) m/z: [M+H] calcd for C₆₀H₇₄FN₇O₉: 1056.6; found 1056.5.

Step 12. To a solution of (3aS,6S,9S,15S,32aS)-21-(5-(benzyloxy)-2-((S)-1-methoxyethyl)pyridin-3-yl)-22-ethyl-1-(2-fluoro-2-methylpropanoyl)-6-isopropyl-5,19,19-trimethyl-3,3a,5,6,8,9,14,15,18,19,20,22,32,32a-tetradecahydro-1H,12H-11,15-epimino-23,25-etheno-9,28-methano-26,30-(metheno)dipyrrolo[2,3-c:3′,4′-v][1,18]dioxa[6,9,12]triazacyclotriacontine-4,7,10,16(2H,13H)-tetraone (145 mg, 0.137 mmol) in MeOH (3.0 mL) was added Pd/C (150 mg). The resulting mixture was stirred for 2.5 h at room temperature under an atmosphere of H₂, filtered, and concentrated under reduced pressure to afford (3aS,6S,9S,15S,32aS)-22-ethyl-1-(2-fluoro-2-methylpropanoyl)-21-(5-hydroxy-2-((S)-1-methoxyethyl)pyridin-3-yl)-6-isopropyl-5,19,19-trimethyl-3,3a,5,6,8,9,14,15,18,19,20,22,32,32a-tetradecahydro-1H,12H-11,15-epimino-23,25-etheno-9,28-methano-26,30-(metheno)dipyrrolo[2,3-c:3′,4′-v][1,18]dioxa[6,9,12]triazacyclotriacontine-4,7,10,16(2H,13H)-tetraone (124 mg, crude) which was used in the next reaction without further purification. LCMS (ESI) m/z: [M+H] calcd for C₅₃H₆₈FN₇O₉: 966.5; found 966.6.

Step 13. To a solution of (3aS,6S,9S,15S,32aS)-22-ethyl-1-(2-fluoro-2-methylpropanoyl)-21-(5-hydroxy-2-((S)-1-methoxyethyl)pyridin-3-yl)-6-isopropyl-5,19,19-trimethyl-3,3a,5,6,8,9,14,15,18,19,20,22,32,32a-tetradecahydro-1H,12H-11,15-epimino-23,25-etheno-9,28-methano-26,30-(metheno)dipyrrolo[2,3-c:3′,4′-v][1,18]dioxa[6,9,12]triazacyclotriacontine-4,7,10,16(2H,13H)-tetraone (60 mg, crude) and K₂CO₃ (34.3 mg, 0.248 mmol) in DMF (2.0 mL) stirred at 0° C. was added 3-(bromomethyl)pyridine hydrobromide (31.4 mg, 0.124 mmol). The resulting mixture was stirred for 2 h at room temperature and quenched at 0° C. by the addition of cold H₂O. The aqueous mixture was extracted with EtOAc (3×20 mL), and the combined organic extracts were washed with brine (3×10 mL), dried over Na₂SO₄, filtered, and concentrated under reduced pressure. Purification by reversed phase chromatography afforded (3aS,6S,9S,15S,32aS)-22-ethyl-1-(2-fluoro-2-methylpropanoyl)-6-isopropyl-21-(2-((S)-1-methoxyethyl)-5-(pyridin-3-ylmethoxy)pyridin-3-yl)-5,19,19-trimethyl-3,3a,5,6,8,9,14,15,18,19,20,22,32,32a-tetradecahydro-1H,12H-11,15-epimino-23,25-etheno-9,28-methano-26,30-(metheno)dipyrrolo[2,3-c:3′,4′-v][1,18]dioxa[6,9,12]triazacyclotriacontine-4,7,10,16(2H,13H)-tetraone (9.2 mg, 13% yield over 2 steps) as a white solid. LCMS (ESI) m/z: [M+H] calcd for C₅₉H₇₃FN₈O₉: 1057.6; found 1057.5; ¹H NMR (400 MHz, DMSO-d₆) δ 8.69 (s, 1H), 8.57-8.53 (m, 2H), 8.06-7.97 (m, 1H), 7.91-7.85 (m, 1H), 7.79-7.51 (m, 2H), 7.51-7.39 (m, 3H), 7.28 (s, 1H), 7.28-7.10 (m, 1H), 6.89-6.52 (m, 1H), 5.51-4.91 (m, 3H), 4.73-4.61 (m, 1H), 4.54-3.93 (m, 7H), 3.92-3.51 (m, 6H), 3.20 (s, 3H), 3.10 (s, 2H), 3.01-2.71 (m, 3H), 2.70 (s, 1H), 2.69-2.53 (m, 2H), 2.44 (s, 2H), 2.32-1.95 (m, 3H), 1.91-1.81 (m, 1H), 1.79-1.51 (m, 7H), 1.49-1.41 (m, 2H), 1.41-1.31 (m, 3H), 1.30-1.16 (m, 2H), 1.00-0.84 (m, 4H), 0.79 (m, 3H), 0.69 (d, J=7.3 Hz, 3H), 0.50 (s, 2H), 0.37 (s, 1H).

### Example A355. Synthesis of (9S,13S,16S,19S,25S)-32-[5-(4-cyclopropylpiperazin-1-yl)-2-[(1S)-1-methoxyethyl]-3-pyridyl]-33-ethyl-16-isopropyl-15,29,29-trimethyl-14,17,20,26-tetraoxo-7,27-dioxa-10,15,18,21,33,39-hexazaheptacyclo[29.5.2.1^{2,6}.1^{4,19}.1^{21,25}.0^{9,13}.0^{34,38}]hentetraconta-1(37),2(41),3,5,31,34(38),35-heptaene-10-carbaldehyde

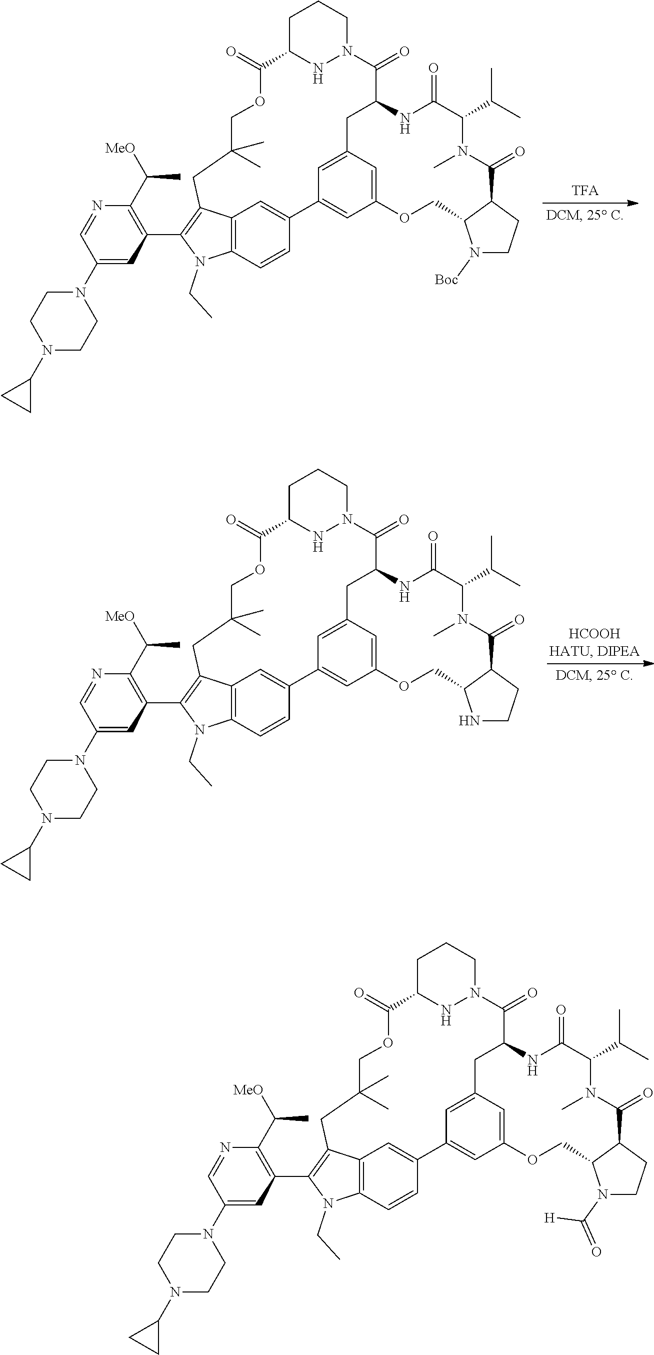

Step 1. A mixture of tert-butyl (9S,13S,16S,19S,25S)-32-[5-(4-cyclopropylpiperazin-1-yl)-2-[(1 S)-1-methoxyethyl]-3-pyridyl]-33-ethyl-16-isopropyl-15,29,29-trimethyl-14,17,20,26-tetraoxo-7,27-dioxa-10,15,18,21,33,39-hexazaheptacyclo[29.5.2.1^{2,6}.1^{4,19}.1^{21,25}.0^{9,13}.0^{34,38}]hentetraconta-1(37),2(41),3,5,31,34(38),35-heptaene-10-carboxylate (50.0 mg, 0.0460 mmol) in DCM (0.5 mL) and TFA (0.25 mL) was stirred at 25° C. for 1 h. The mixture was then treated with sat. aq. NaHCO₃ (3 mL), extracted with DCM (3×1 mL), washed with brine (1 mL), dried over Na₂SO₄, and concentrated under reduce pressure to give (9S,13S,16S,19S,25S)-32-[5-(4-cyclopropylpiperazin-1-yl)-2-[(1 S)-1-methoxyethyl]-3-pyridyl]-33-ethyl-16-isopropyl-15,29,29-trimethyl-7,27-dioxa-10,15,18,21,33,39-hexazaheptacyclo[29.5.2.1^{2,6}.1^{4,19}.1^{21,25}.0^{9,13}.0^{34,38}]hentetraconta-1(37),2(41),3,5,31,34(38),35-heptaene-14,17,20,26-tetrone (40 mg, crude) as yellow solid. LCMS (ESI) m/z: [M+H] calcd for C₅₆H₇₅N₉O₇: 986.6; found 986.8.

Step 2. To a stirred mixture of (9S,13S,16S,19S,25S)-32-[5-(4-cyclopropylpiperazin-1-yl)-2-[(1 S)-1-methoxyethyl]-3-pyridyl]-33-ethyl-16-isopropyl-15,29,29-trimethyl-7,27-dioxa-10,15,18,21,33,39-hexazaheptacyclo[29.5.2.1^{2,6}.1^{4,19}.1^{21,25}.0^{9,13}.0^{34,38}]hentetraconta-1(37),2(41),3,5,31,34(38),35-heptaene-14,17,20,26-tetrone (15 mg, crude) in DCM (0.8 mL) was added formic acid (1.5 mg, 0.030 mmol), HATU (6.94 mg, 0.0183 mmol) and DIPEA (5.90 mg, 0.0456 mmol) at 0° C. The reaction mixture was stirred at 25° C. for 4 h and was then concentrated under reduced pressure. The residue was purified by reversed phase prep-HPLC to give (9S,13S,16S,19S,25S)-32-[5-(4-cyclopropylpiperazin-1-yl)-2-[(1 S)-1-methoxyethyl]-3-pyridyl]-33-ethyl-16-isopropyl-15,29,29-trimethyl-14,17,20,26-tetraoxo-7,27-dioxa-10,15,18,21,33,39-hexazaheptacyclo[29.5.2.1^{2,6}.1^{4,19}.1^{21,25}.0^{9,13}.0^{34,38}]hentetraconta-1(37),2(41),3,5,31,34(38),35-heptaene-10-carbaldehyde (2.0 mg, 11% yield over 2 steps) as a white solid. LCMS (ESI) m/z: [M+H] calcd for C₅₇H₇₅N₉O₈: 1014.6; found 1014.7. ¹H NMR (400 MHz, DMSO-d₆) 8.49-8.43 (m, 1H), 8.40-8.32 (m, 1H), 8.31-8.10 (m, 1H), 8.04-7.93 (m, 1H), 7.74-7.67 (m, 1H), 7.62-7.48 (m, 2H), 7.47-7.33 (m, 2H), 7.28-7.09 (m, 3H), 6.82-6.59 (m, 2H), 6.20-6.06 (m, 1H), 5.38-5.11 (m, 2H), 4.83-4.67 (m, 1H), 4.57-4.42 (m, 2H), 4.34-4.19 (m, 4H), 4.17-4.00 (m, 5H), 3.71-3.63 (m, 3H), 3.23 (d, J=8.4 Hz, 6H), 3.12 (s, 3H), 3.07-3.01 (m, 2H), 1.03-0.93 (m, 5H), 0.89-0.78 (m, 8H), 0.75 (d, J=6.8 Hz, 4H), 0.69 (d, J=6.8 Hz, 1H), 0.57 (s, 1H), 0.37-0.29 (m, 2H).

### Example A361 Synthesis of (3aS,6S,9S,15S,32aS)-21-(5-(4-cyclopropylpiperazin-1-yl)-2-((S)-1-methoxyethyl)pyridin-3-yl)-1-(dimethylphosphoryl)-22-ethyl-6-isopropyl-5,19,19-trimethyl-3,3a,5,6,8,9,14,15,18,19,20,22,32,32a-tetradecahydro-1H,12H-11,15-epimino-23,25-etheno-9,28-methano-26,30-(metheno)dipyrrolo[2,3-c:3′,4′-v][1,18]dioxa[6,9,12]triazacyclotriacontine-4,7,10,16(2H,13H)-tetraone

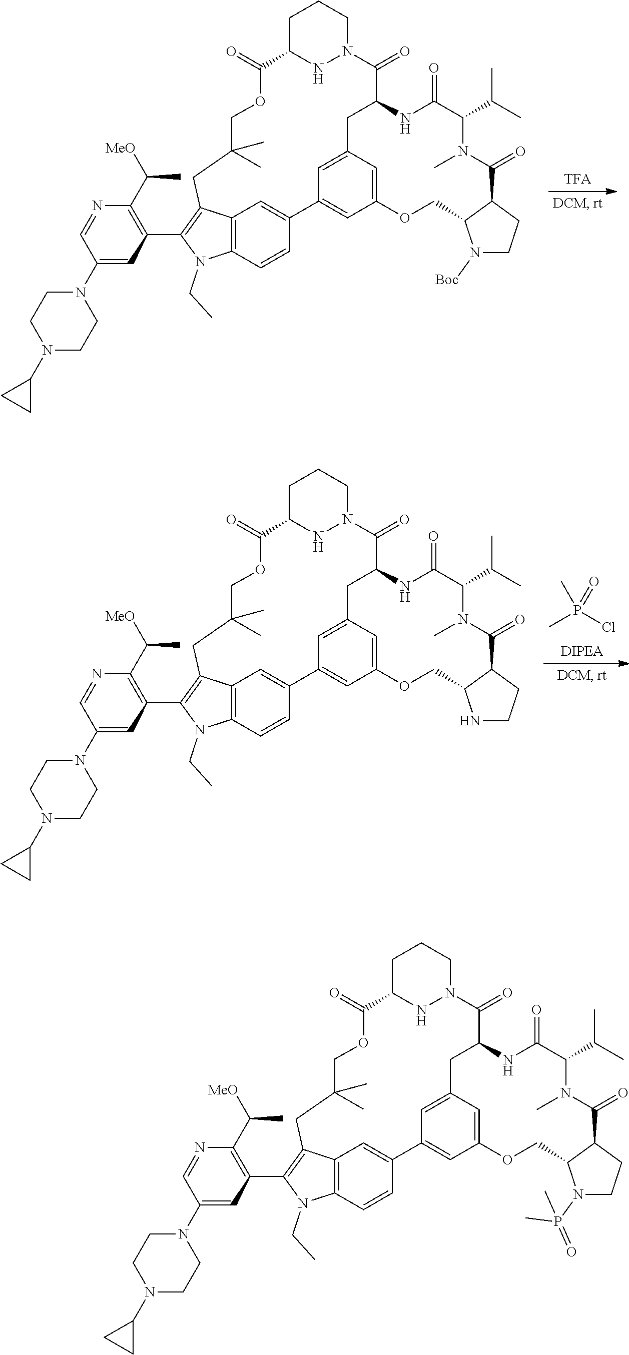

Step 1. To a stirred solution of tert-butyl (9S,13S,16S,19S,25S)-32-[5-(4-cyclopropylpiperazin-1-yl)-2-[(1 S)-1-methoxyethyl]-3-pyridyl]-33-ethyl-16-isopropyl-15,29,29-trimethyl-14,17,20,26-tetraoxo-7,27-dioxa-10,15,18,21,33,39-hexazaheptacyclo[29.5.2.1^{2,6}.1^{4,19}.1^{21,25}.0^{9,13}.0^{34,38}]hentetraconta-1(37),2(41),3,5,31,34(38),35-heptaene-10-carboxylate (50 mg, 46.0 μmol) in DCM (0.67 mL) was added TFA (0.33 mL). The reaction mixture was stirred at 25° C. for 1 h and was then poured into H₂O (1.0 mL). The resulting mixture was neutralized to pH 9 with sat. aq. NaHCO₃, extracted with DCM (3×mL), washed with brine (2.0 mL), dried over anhydrous Na₂SO₄, filtered, and concentrated under reduced pressure to give (9S,13S,16S,19S,25S)-32-[5-(4-cyclopropylpiperazin-1-yl)-2-[(1 S)-1-methoxyethyl]-3-pyridyl]-33-ethyl-16-isopropyl-15,29,29-trimethyl-7,27-dioxa-10,15,18,21,33,39-hexazaheptacyclo[29.5.2.1^{2,6}.1^{4,19}.1^{21,25}.0^{9,13}.0^{34,38}]hentetraconta-1(37),2(41),3,5,31,34(38),35-heptaene-14,17,20,26-tetrone (50 mg, crude) as a yellow solid. LCMS (ESI) m/z: [M+H] calcd for C₅₆H₇₅N₉O₇: 986.6; found 986.7.

Step 2. To a stirred solution (9S,13S,16S,19S,25S)-32-[5-(4-cyclopropylpiperazin-1-yl)-2-[(1S)-1-methoxyethyl]-3-pyridyl]-33-ethyl-16-isopropyl-15,29,29-trimethyl-7,27-dioxa-10,15,18,21,33,39-hexazaheptacyclo[29.5.2.1^{2,6}.1^{4,19}.1^{21,25}.0^{9,13}.0^{34,38}]hentetraconta-1(37),2(41),3,5,31,34(38),35-heptaene-14,17,20,26-tetrone (50 mg, crude) in DCM (1 mL) were added DIPEA (26.2 mg, 203 μmol) and dimethylphosphinic chloride (8.55 mg, 76.1 μmol). The reaction mixture was stirred at 25° C. for 15 min. The resulting mixture was treating with sat. aq. NaHCO₃ (1 mL), extracted with DCM (3×1 mL), washed with brine (1 mL), dried over anhydrous Na₂SO₄, filtered, and concentrated under reduced pressure. The residue was reversed phase prep-HPLC to give (3aS,6S,9S,15S,32aS)-21-(5-(4-cyclopropylpiperazin-1-yl)-2-((S)-1-methoxyethyl)pyridin-3-yl)-1-(dimethylphosphoryl)-22-ethyl-6-isopropyl-5,19,19-trimethyl-3,3a,5,6,8,9,14,15,18,19,20,22,32,32a-tetradecahydro-1H,12H-11,15-epimino-23,25-etheno-9,28-methano-26,30-(metheno)dipyrrolo[2,3-c:3′,4′-v][1,18]dioxa[6,9,12]triazacyclotriacontine-4,7,10,16(2H,13H)-tetraone (6.0 mg, 12% yield over 2 steps) as a white solid. LCMS (ESI) m/z: [M+H] calcd for C₅₈H₈₀N₉O₈P: 1062.6; found 1062.6. ¹H NMR (400 MHz, DMSO-d₆) δ 8.43-8.42 (d, J=2.6 Hz, 1H) 7.93-8.29 (m, 1H) 7.76-7.65 (m, 1H) 7.59-7.52 (m, 1H) 7.50-7.30 (m, 2H) 7.25-7.13 (m, 2H) 6.71-6.59 (m, 1H) 6.12 (s, 1H) 5.27-5.14 (m, 1H) 4.82-4.73 (m, 1H) 4.45-4.42 (d, J=11.3 Hz, 1H) 4.32-4.18 (m, 3H) 4.16-4.01 (m, 3H) 3.94-3.92 (d, J=8.3 Hz, 1H) 3.75-3.62 (m, 2H) 3.59-3.43 (m, 2H) 3.28-3.15 (m, 6H) 3.15-3.04 (m, 3H) 2.94-2.76 (m, 3H) 2.72 (s, 1H) 2.69-2.57 (m, 6H) 2.32-2.17 (m, 1H) 2.15-1.98 (m, 3H) 1.85-1.82 (d, J=11.8 Hz, 1H) 1.77-1.62 (m, 3H) 1.47-1.32 (m, 8H) 1.31-1.23 (m, 2H) 1.09-0.65 (m, 12H) 0.56-0.41 (m, 5H) 0.34-0.33 (d, J=2.4 Hz, 2H).

### Example A362. Synthesis of (4aS,7S,10S,16S,33aS)-22-(5-(4-cyclopropylpiperazin-1-yl)-2-((S)-1-methoxyethyl)pyridin-3-yl)-23-ethyl-1-(2-fluoro-2-methylpropanoyl)-7-isopropyl-6,20,20-trimethyl-2,3,4,4a,6,7,9,10,15,16,19,20,21,23,33,33a-hexadecahydro-13H-12,16-epimino-24,26-etheno-10,29-methano-27,31-(metheno)pyrido[2,3-c]pyrrolo[3,4-v][1,18]dioxa[6,9,12]triazacyclotriacontine-5,8,11,17(1H,14H)-tetraone

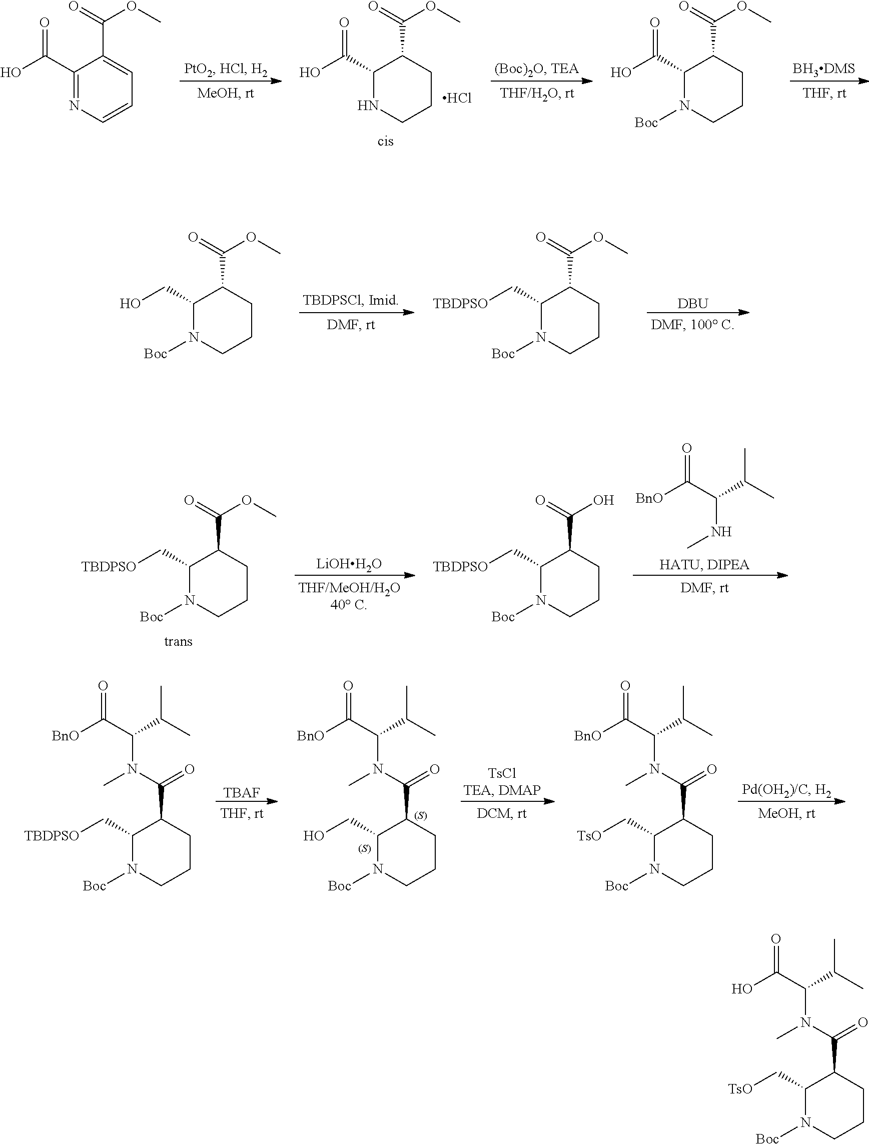

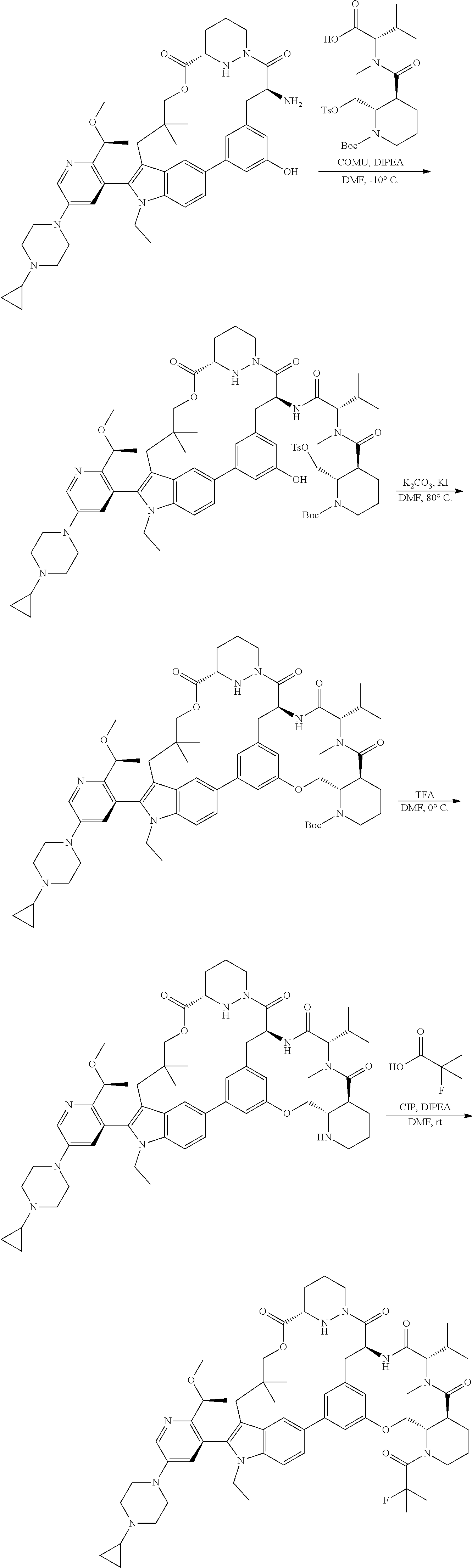

Step 1. To a solution of 3-(methoxycarbonyl)picolinic acid (14.7 g, 81.1 mmol) in MeOH (135 mL) and 1M aq. HCl (15 mL) was added PtO₂ (9.2 g, 40.5 mmol). The resulting mixture was stirred overnight at room temperature under an atmosphere of H₂, filtered, and the filter cake washed with MeOH (3×150 mL). The filtrate was concentrated under reduced pressure to afford cis-3-(methoxycarbonyl)piperidine-2-carboxylic acid hydrochloride (20.4 g, crude) which was taken directly to the next reaction without further purification. LCMS (ESI) m/z: [M+H] calcd for C₈H₁₃NO₄: 188.1; found 188.1.

Step 2. To a solution of cis-3-(methoxycarbonyl)piperidine-2-carboxylic acid hydrochloride (20.4 g, crude) in THE (100 mL) and H₂O (100 mL) stirred at 0° C. was added Et₃N (110 g, 1089 mmol) and (Boc)₂O (119 g, 545 mmol). The resulting solution was stirred overnight at room temperature and was then diluted with H₂O (20 mL). The aqueous mixture was extracted with EtOAc (3×150 mL), and the combined organic extracts were dried with Na₂SO₄, filtered, and concentrated under reduced pressure. Purification by reversed phase chromatography afforded 1-(tert-butoxycarbonyl)-3-(methoxycarbonyl)piperidine-2-carboxylic acid (8.6 g, 37% yield over 2 steps) as a white solid. LCMS (ESI) m/z: [2M+Na] calcd for C₁₃H₂₁NO₆: 597.3; found 597.3.

Step 3. To a solution of cis-1-(tert-butoxycarbonyl)-3-(methoxycarbonyl)piperidine-2-carboxylic acid (8.8 g, 30.6 mmol) in THE (10 mL) at −78° C. was added BH₃₀DMS (4.6 mL, 45.9 mmol). The resulting mixture was stirred for 2 h at −78° C. and was then quenched at 0° C. by the addition of MeOH (50 mL). The resulting solution was stirred for 1 h at room temperature and was then concentrated under reduced pressure to afford cis-1-(tert-butyl) 3-methyl 2-(hydroxymethyl)piperidine-1,3-dicarboxylate (8.0 g, crude) which was taken directly to the next reaction without further purification. LCMS (ESI) m/z: [M+H] calcd for C₁₃H₂₃NO₅: 274.2; found 274.3.

Step 4. To a solution of cis-1-(tert-butyl) 3-methyl 2-(hydroxymethyl)piperidine-1,3-dicarboxylate (8.0 g, crude) in DCM (110 mL) at 0° C. was added imidazole (5.83 g, 85.8 mmol) and TBDPSCI (14.1 g, 51.5 mmol). The resulting mixture was stirred for 1 h at room temperature and was then quenched at 0° C. by the addition of H₂O. The aqueous phase was extracted with DCM (3×150 mL), and the combined organic extracts were dried over Na₂SO₄, filtered, and concentrated under reduced pressure. Purification by reversed phase chromatography afforded cis-1-(tert-butyl) 3-methyl 2-(((tert-butyldiphenylsilyl)oxy)methyl)piperidine-1,3-dicarboxylate (11.6 g, 74% yield over 2 steps) as a white solid. LCMS (ESI) m/z: [M+H] calcd for C₂₉H₄₁NO₅Si: 512.3; found 512.3.

Step 5. To a solution of cis-1-(tert-butyl) 3-methyl 2-(((tert-butyldiphenylsilyl)oxy)methyl)piperidine-1,3-dicarboxylate (10.6 g, 20.7 mmol) in DMF (100 mL) was added DBU (15.8 g, 103 mmol). The resulting mixture was stirred overnight at 100° C. and was then quenched at 0° C. by the addition of H₂O (20 mL). The aqueous mixture was extracted with pet. ether (3×50 mL), and the combined organic extracts were dried over Na₂SO₄, filtered, and concentrated under reduced pressure. Purification by reversed phase chromatography afforded trans-1-(tert-butyl)-3-methyl-2-(((tert-butyldiphenylsilyl)oxy)methyl)piperidine-1,3-dicarboxylate (5.65 g, 52% yield) as a clear oil. LCMS (ESI) m/z: [M+NH₄] calcd for C₂₉H₄₁NO₅Si: 529.3; found 529.4.

Step 6. To a solution of trans-1-(tert-butyl)-3-methyl-2-(((tert-butyldiphenylsilyl)oxy)methyl)piperidine-1,3-dicarboxylate (5.65 g, 11.0 mmol) in THE (20 mL) and MeOH (20 mL) stirred at 0° C. was added a solution of LiOH·H₂O (927 mg, 22.0 mmol) in H₂O (20 mL). The resulting mixture was stirred for 8 h at 40° C. and was then acidified to pH=6 by the addition of 1M aq. HCl. The aqueous mixture was extracted with EtOAc (3×50 mL), and the combined organic extracts were dried over Na₂SO₄, filtered, and concentrated under reduced pressure to afford the desired product (5.1 g, crude) which was taken directly to the next reaction without further purification. LCMS (ESI) m/z: [M+Na] calcd for C₂₅H₃₉NO₅Si: 520.2; found 520.1.

Step 7. To a solution of 1-(tert-butoxycarbonyl)-2-(((tert-butyldiphenylsilyl)oxy)methyl)piperidine-3-carboxylic acid (5.1 g, crude) in DMF (50 mL) at 0° C. was added DIPEA (6.62 g, 51.2 mmol), benzyl methyl-L-valinate (2.72 g, 12.3 mmol) and a solution of HATU (7.79 g, 20.5 mmol) in DMF (10 mL). The resulting mixture was stirred for 8 h at room temperature and was then quenched at 0° C. by the addition of H₂O (50 mL). The aqueous mixture was extracted with EtOAc (3×150 mL), and the combined organic extracts were washed with brine (3×80 mL), dried over Na₂SO₄, filtered, and concentrated under reduced pressure. Purification by normal phase chromatography afforded trans-tert-butyl 3-(((S)-1-(benzyloxy)-3-methyl-1-oxobutan-2-yl)(methyl)carbamoyl)-2-(((tert-butyldiphenylsilyl)oxy)methyl)piperidine-1-carboxylate (4.72 g, 61% yield over 2 steps) as a yellow oil. LCMS (ESI) m/z: [M+H] calcd for C₄₁H₅₆N₂O₆Si: 701.4; found 701.2.

Step 8. To a solution of trans-tert-butyl 3-(((S)-1-(benzyloxy)-3-methyl-1-oxobutan-2-yl)(methyl)carbamoyl)-2-(((tert-butyldiphenylsilyl)oxy)methyl)piperidine-1-carboxylate (4.7 g, 6.71 mmol) in THE (60 mL) at 0° C. was added a solution of TBAF (13.4 mL, 13.4 mmol, 1M in THF). The resulting mixture was stirred for 5 h at room temperature and was then quenched at 0° C. by the addition of H₂O (30 mL). The aqueous mixture was extracted with EtOAc (3×50 mL), and the combined organic extracts were dried over Na₂SO₄, filtered, and concentrated under reduced pressure. Purification by reversed phase chromatography afforded a mixture of 2 diastereomers, which were separated by preparative-SFC. Collection of the earlier eluting diastereomer afforded tert-butyl (2S,3S)-3-(((S)-1-(benzyloxy)-3-methyl-1-oxobutan-2-yl)(methyl)carbamoyl)-2-(hydroxymethyl)piperidine-1-carboxylate (1.32 g, 43% yield, assumed configuration) as a clear oil and collection of the later eluting diastereomer afforded (2R,3R)-3-(((S)-1-(benzyloxy)-3-methyl-1-oxobutan-2-yl)(methyl)carbamoyl)-2-(hydroxymethyl)piperidine-1-carboxylate (1.29 g, 42% yield, assumed configuration) as a clear oil. LCMS (ESI) m/z: [M+H] calcd for C₂₅H₃₈N₂O₆: 463.3; found 463.3.

Step 9. To a solution of tert-butyl (2S,3S)-3-(((S)-1-(benzyloxy)-3-methyl-1-oxobutan-2-yl)(methyl)carbamoyl)-2-(hydroxymethyl)piperidine-1-carboxylate (1.35 g, 2.92 mmol) in DCM (14 mL) was added Et₃N (1.18 g, 11.7 mmol), DMAP (71.3 mg, 0.584 mmol) and p-toluenesulfonyl chloride (1.11 g, 5.84 mmol). The resulting mixture was stirred overnight at room temperature and was then quenched at 0° C. by the addition of H₂O (50 mL). The aqueous phase was extracted with DCM (3×50 mL), and the combined organic extracts were dried over Na₂SO₄, filtered, and concentrated under reduced pressure. Purification by normal phase chromatography afforded tert-butyl (2S,3S)-3-(((S)-1-(benzyloxy)-3-methyl-1-oxobutan-2-yl)(methyl)carbamoyl)-2-((tosyloxy)methyl)piperidine-1-carboxylate (1.6 g, 89% yield) as a white solid. LCMS (ESI) m/z: [M+H] calcd for C₃₂H₄₄N₂O₈S: 617.3; found 617.3.

Step 10. To a solution of tert-butyl (2S,3S)-3-(((S)-1-(benzyloxy)-3-methyl-1-oxobutan-2-yl)(methyl)carbamoyl)-2-((tosyloxy)methyl)piperidine-1-carboxylate (1.58 g, 2.56 mmol) in MeOH (16 mL) was added Pd(OH)₂/C (1.58 g). The resulting mixture was stirred for 1 h at room temperature under an atmosphere of H₂, filtered, and the filter cake washed with MeOH (5×30 mL). The filtrate was concentrated under reduced pressure to afford N-((2S,3S)-1-(tert-butoxycarbonyl)-2-((tosyloxy)methyl)piperidine-3-carbonyl)-N-methyl-L-valine (1.2 g, crude) which was used in the next reaction without further purification. LCMS (ESI) m/z: [M+Na] calcd for C₂₅H₃₈N₂O₈S: 549.2; found 549.2.

Step 11. To a solution of (6³S,4S)-4-amino-1²-(5-(4-cyclopropylpiperazin-1-yl)-2-((S)-1-methoxyethyl)pyridin-3-yl)-1¹-ethyl-2⁵-hydroxy-10,10-dimethyl-6¹,6²,6³,6⁴,6⁵,6⁶-hexahydro-1 ¹H-8-oxa-1(5,3)-indola-6(1,3)-pyridazina-2(1,3)-benzenacycloundecaphane-5,7-dione (700 mg, 0.916 mmol) in DMF (7.0 mL) at −10° C. was added DIPEA (4.75 g, 36.6 mmol), N-((2S,3S)-1-(tert-butoxycarbonyl)-2-((tosyloxy)methyl)piperidine-3-carbonyl)-N-methyl-L-valine (627 mg, crude) and COMU (471 mg, 1.10 mmol). The resulting mixture was stirred for 1 h at −10° C., quenched by the addition of H₂O (30 mL), and acidified to pH=7 by the addition of 1M aq. HCl. The aqueous mixture was extracted with EtOAc (3×30 mL), and the combined organic extracts were washed with brine (3×50 mL), dried over Na₂SO₄, filtered, and concentrated under reduced pressure. Purification by normal phase chromatography afforded tert-butyl (2S,3S)-3-(((2S)-1-(((6³S,4S)-1²-(5-(4-cyclopropylpiperazin-1-yl)-2-((S)-1-methoxyethyl)pyridin-3-yl)-1¹-ethyl-2⁵-hydroxy-10,10-dimethyl-5,7-dioxo-6¹,6²,6³,6⁴,6⁵,6⁶-hexahydro-1¹H-8-oxa-1(5,3)-indola-6(1,3)-pyridazina- 2(1,3)-benzenacycloundecaphane-4-yl)amino)-3-methyl-1-oxobutan-2-yl)(methyl)carbamoyl)-2-((tosyloxy)methyl)piperidine-1-carboxylate (900 mg, 62% yield) as a red solid. LCMS (ESI) m/z: [M+H] calcd for C₆₉H₉₃N₉O₁₂S: 1272.7; found 1272.5.

Step 12. To a solution of tert-butyl (2S,3S)-3-(((2S)-1-(((6³S,4S)-1²-(5-(4-cyclopropylpiperazin-1-yl)-2-((S)-1-methoxyethyl)pyridin-3-yl)-1¹-ethyl-2⁵-hydroxy-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)amino)-3-methyl-1-oxobutan-2-yl)(methyl)carbamoyl)-2-((tosyloxy)methyl)piperidine-1-carboxylate (890 mg, 0.699 mmol) in DMF (90 mL) stirred at room temperature was added K₂CO₃ (967 mg, 6.99 mmol) and KI (116.09 mg, 0.699 mmol). The resulting solution was stirred for 1 h at 80° C. and was then quenched at 0° C. by the addition of H₂O (200 mL). The aqueous mixture was extracted with EtOAc (3×200 mL), and the combined organic extracts were washed with brine (3×400 mL), dried over Na₂SO₄, filtered, and concentrated under reduced pressure. Purification by reversed phase chromatography afforded tert-butyl (4aS,7S,10S,16S,33aS)-22-(5-(4-cyclopropylpiperazin-1-yl)-2-((S)-1-methoxyethyl)pyridin-3-yl)-23-ethyl-7-isopropyl-6,20,20-trimethyl-5,8,11,17-tetraoxo-3,4,4a,5,6,7,8,9,10,11,14,15,16,17,19,20,21,23,33,33a-icosahydro-13H-12,16-epimino-24,26-etheno-10,29-methano-27,31-(metheno)pyrido[2,3-c]pyrrolo[3,4-v][1,18]dioxa[6,9,12]triazacyclotriacontine-1(2H)-carboxylate (240 mg, 31% yield) as a white solid. LCMS (ESI) m/z: [M+H] calcd for C₆₂H₈₅N₉O₉: 1100.7; found 1100.8.

Step 13. To a solution of tert-butyl (4aS,7S,10S,16S,33aS)-22-(5-(4-cyclopropylpiperazin-1-yl)-2-((S)-1-methoxyethyl)pyridin-3-yl)-23-ethyl-7-isopropyl-6,20,20-trimethyl-5,8,11,17-tetraoxo-3,4,4a,5,6,7,8,9,10,11,14,15,16,17,19,20,21,23,33,33a-icosahydro-13H-12,16-epimino-24,26-etheno-10,29-methano-27,31-(metheno)pyrido[2,3-c]pyrrolo[3,4-v][1,18]dioxa[6,9,12]triazacyclotriacontine-1(2H)-carboxylate (235 mg, 0.214 mmol) in DCM (5.0 mL) stirred at 0° C. was added TFA (1.0 mL). The resulting mixture was stirred for 30 min at 0° C., concentrated under reduced pressure, and the concentrate basified to pH=8 by the addition of sat. aq. NaHCO₃. The aqueous mixture was extracted with DCM (3×20 mL) and the combined organic extracts were dried over Na₂SO₄, filtered, and concentrated under reduced pressure to afford (4aS,7S,10S,16S,33aS)-22-(5-(4-cyclopropylpiperazin-1-yl)-2-((S)-1-methoxyethyl)pyridin-3-yl)-23-ethyl-7-isopropyl-6,20,20-trimethyl-2,3,4,4a,6,7,9,10,15,16,19,20,21,23,33,33a-hexadecahydro-13H-12,16-epimino-24,26-etheno-10,29-methano-27,31-(metheno)pyrido[2,3-c]pyrrolo[3,4-v][1,18]dioxa[6,9,12]triazacyclotriacontine-5,8,11,17(1H,14H)-tetraone (210 mg, crude) which was used in the next reaction without further purification. LCMS (ESI) m/z: [M+H] calcd for C₅₇H₇₇N₉O₇: 1000.6; found 1000.7.

Step 14. To a solution of (4aS,7S,10S,16S,33aS)-22-(5-(4-cyclopropylpiperazin-1-yl)-2-((S)-1-methoxyethyl)pyridin-3-yl)-23-ethyl-7-isopropyl-6,20,20-trimethyl-2,3,4,4a,6,7,9,10,15,16,19,20,21,23,33,33a-hexadecahydro-13H-12,16-epimino-24,26-etheno-10,29-methano-27,31-(metheno)pyrido[2,3-c]pyrrolo[3,4-v][1,18]dioxa[6,9,12]triazacyclotriacontine-5,8,11,17(1H,14H)-tetraone (210 mg, crude) in DMF (3.0 mL) stirred at 0° C. was added DIPEA (284 mg, 2.20 mmol), 2-fluoro-2-methylpropanoic acid (35.0 mg, 0.330 mmol), and CIP (79.7 mg, 0.286 mmol). The resulting solution was stirred for 1 h at room temperature and was then quenched at 0° C. by the addition of H₂O (10 mL). The aqueous mixture was extracted with EtOAc (3×20 mL), and the combined organic extracts were washed with brine (3×40 mL), dried over Na₂SO₄, filtered, and concentrated under reduced pressure. Purification by reversed phase chromatography afforded (4aS,7S,10S,16S,33aS)-22-(5-(4-cyclopropylpiperazin-1-yl)-2-((S)-1-methoxyethyl)pyridin-3-yl)-23-ethyl-1-(2-fluoro-2-methylpropanoyl)-7-isopropyl-6,20,20-trimethyl-2,3,4,4a,6,7,9,10,15,16,19,20,21,23,33,33a-hexadecahydro-13H-12,16-epimino-24,26-etheno-10,29-methano-27,31-(metheno)pyrido[2,3-c]pyrrolo[3,4-v][1,18]dioxa[6,9,12]triazacyclotriacontine-5,8,11,17(1H,14H)-tetraone (68.1 mg, 29% yield over 2 steps) as a white solid. LCMS (ESI) m/z: [M+H] calcd for C₆₁H₈₂FN₉O₈: 1088.6; found 1088.6; ¹H NMR (300 MHz, DMSO-d₆) δ 8.47 (br s, 2H), 7.93 (s, 1H), 7.71-7.45 (m, 2H), 7.41-6.99 (m, 3H), 6.61 (s, 1H), 5.36-5.20 (m, 1H), 5.05 (d, J=12.2 Hz, 1H), 4.90 (d, J=11.3 Hz, 1H), 4.49 (d, J=10.9 Hz, 1H), 4.41-3.99 (m, 9H), 3.86-3.57 (m, 5H), 3.30-2.95 (m, 11H), 2.78-2.60 (m, 5H), 2.34 (d, J=14.7 Hz, 2H), 2.11 (br s, 3H), 1.83 (br s, 4H), 1.62-1.42 (m, 8H), 1.40-1.17 (m, 4H), 1.06-0.54 (m, 12H), 0.52-0.29 (m, 6H).

### Example A363. Synthesis of (3aS,6S,9S,15S,32aS)-21-(5-(4-cyclopropylpiperazin-1-yl)-2-((S)-1-meth oxyethyl)pyridin-3-yl)-22-ethyl-1-(2-fluoro-2-methylpropanoyl)-6-isopropyl-5,19,19-trimethyl-2,3,3a,5,6,8,9,14,15,18,19,20,22,31,32,32a-hexadecahydro-12H-11,15-epimino-23,25-etheno-9,28-methano-26,30-(metheno)dipyrrolo[3,2-o:3′,4′-z][1]oxa[7,10,13,18]tetraazacyclotriacontine-4,7,10,16(1H,13H)-tetraone

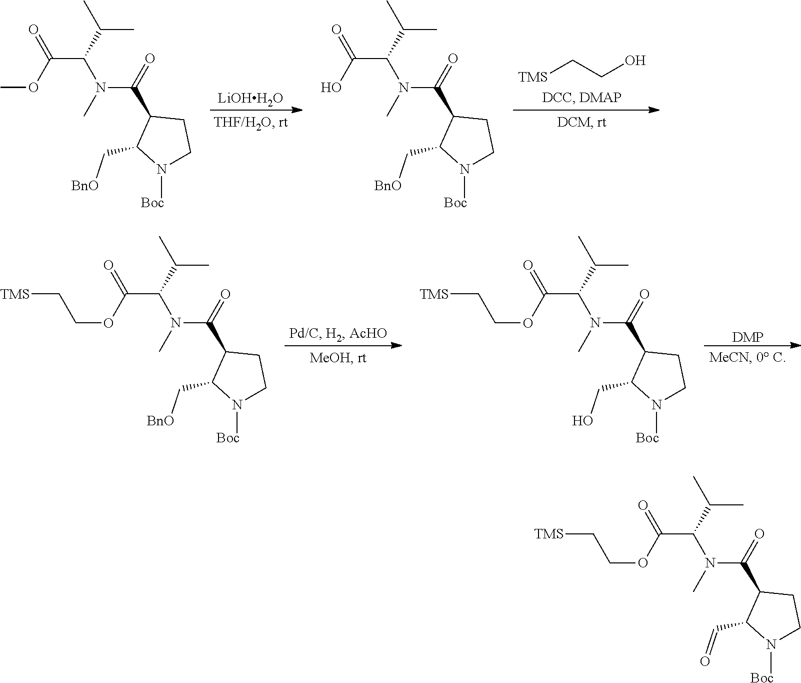

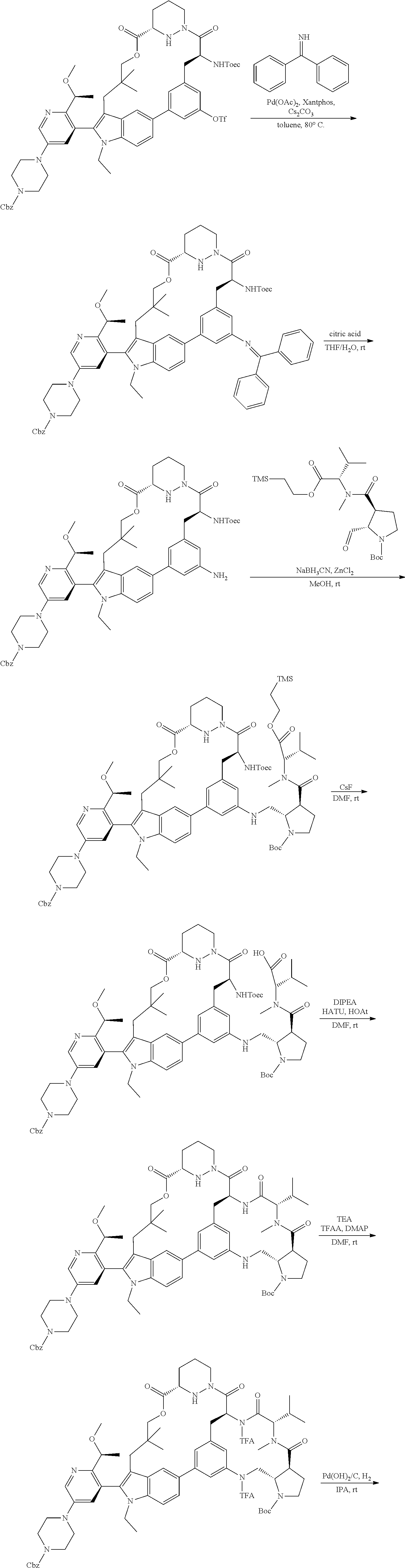

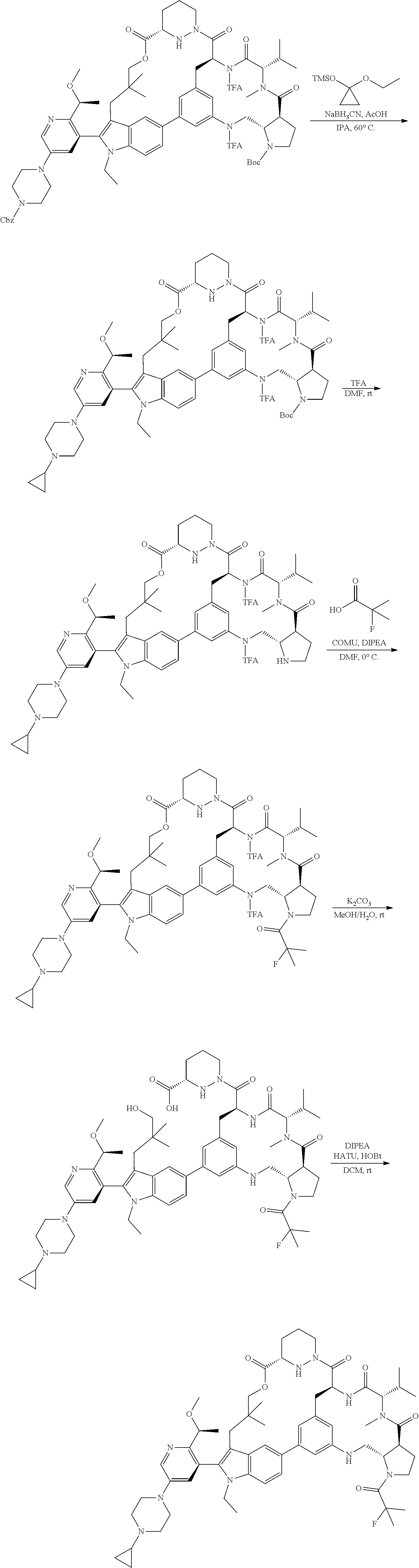

Step 1. To a solution of tert-butyl (2S,3S)-2-((benzyloxy)methyl)-3-(((S)-1-methoxy-3-methyl-1-oxobutan-2-yl)(methyl)carbamoyl)pyrrolidine-1-carboxylate (2.43 g, 5.25 mmol) in THE (12 mL) and H₂O (12 mL) at 0° C. was added LiOH·H₂O (441 mg, 10.5 mmol). The resulting mixture was stirred for 2 h at room temperature then acidified to pH=5 with 1M aq. HCl. The aqueous mixture was extracted with EtOAc (3×30 mL), and the combined organic extracts were dried over Na₂SO₄, filtered, and concentrated under reduced pressure to afford N-((2S,3S)-2-((benzyloxy)methyl)-1-(tert-butoxycarbonyl)pyrrolidine-3-carbonyl)-N-methyl-L-valine (2.5 g, crude) which was taken to the next reaction without further purification. LCMS (ESI) m/z: [M+H−Boc] calcd for C₂₄H₃₆N₂O₆: 349.3; found 349.2.

Step 2. To a solution of N-((2S,3S)-2-((benzyloxy)methyl)-1-(tert-butoxycarbonyl)pyrrolidine-3-carbonyl)-N-methyl-L-valine (1.95 g, 4.34 mmol) and 2-(trimethylsilyl)ethan-1-ol (1.03 g, 8.69 mmol) in DCM (20 mL) at 0° C. were added DMAP (530 mg, 4.35 mmol) and DCC (2.69 g, 13.0 mmol). The resulting mixture was stirred for 1 h at room temperature and was then quenched at 0° C. by the addition of H₂O. The aqueous mixture was extracted with DCM (3×30 mL), and the combined organic extracts were dried over Na₂SO₄, filtered, and concentrated under reduced pressure. Purification by normal phase chromatography afforded tert-butyl (2S,3S)-2-((benzyloxy)methyl)-3-(methyl((S)-3-methyl-1-oxo-1-(2-(trimethylsilyl)ethoxy)butan-2-yl)carbamoyl)pyrrolidine-1-carboxylate (1.9 g, 79% yield) as a yellow oil. LCMS (ESI) m/z: [M+Na] calcd for C₂₉H₄₈N₂O₆Si: 571.3; found 571.2.

Step 3. To a solution of tert-butyl (2S,3S)-2-((benzyloxy)methyl)-3-(methyl((S)-3-methyl-1-oxo-1-(2-(trimethylsilyl)ethoxy)butan-2-yl)carbamoyl)pyrrolidine-1-carboxylate (2.0 g, 3.6 mmol) in MeOH (18 mL) and acetic acid (2.0 mL) at room temperature was added Pd/C (2.0 g). The resulting mixture was stirred overnight under an atmosphere of H₂, filtered, and the filter cake washed with MeOH (3×30 mL). The resulting filtrate was concentrated under reduced pressure to afford tert-butyl (2S,3S)-2-(hydroxymethyl)-3-(methyl((S)-3-methyl-1-oxo-1-(2-(trimethylsilyl)ethoxy)butan-2-yl)carbamoyl)pyrrolidine-1-carboxylate (1.5 g, crude), which was taken to the next step without further purification. LCMS (ESI) m/z: [M+Na] calcd for C₂₂H₄₂N₂O₆Si: 481.3; found 481.2.

Step 4. To a solution of tert-butyl (2S,3S)-2-(hydroxymethyl)-3-(methyl((S)-3-methyl-1-oxo-1-(2-(trimethylsilyl)ethoxy)butan-2-yl)carbamoyl)pyrrolidine-1-carboxylate (1.4 g, crude) in MeCN (15 mL) at 0° C. was added Dess-Martin periodinane (2.98 g, 7.02 mmol). The resulting mixture was stirred for 4 h at 0° C. and was then quenched by the addition of sat. aq. Na₂S₂O₃ (30 mL). The aqueous mixture was extracted with DCM (3×30 mL), and the combined organic extracts were dried over Na₂SO₄, filtered, and concentrated under reduced pressure to afford tert-butyl (2S,3S)-2-formyl-3-(methyl((S)-3-methyl-1-oxo-1-(2-(trimethylsilyl)ethoxy)butan-2-yl)carbamoyl)pyrrolidine-1-carboxylate (1.4 g, crude) which was taken to the next step without further purification. LCMS (ESI) m/z: [M+Na] calcd for C₂₂H₄₀N₂O₆Si: 479.3; found 479.2.

Step 5. To a solution of benzyl 4-(5-((63S,4S)-1¹-ethyl-10,10-dimethyl-5,7-dioxo-2⁵-(((trifluoromethyl)sulfonyl)oxy)-4-(((2-(trimethylsilyl)ethoxy)carbonyl)amino)-6¹,6²,6³,6⁴,6⁵,6⁶-hexahydro-11H-8-oxa-1(5,3)-indola-6(1,3)-pyridazina-2(1,3)-benzenacycloundecaphane-1²-yl)-6-((S)-1-methoxyethyl)pyridin-3-yl)piperazine-1-carboxylate (2.5 g, crude) and diphenylmethanimine (799 mg, 4.41 mmol) in toluene (20 mL) were added Cs₂CO₃ (2.87 g, 8.82 mmol), Pd(OAc)₂ (197 mg, 0.882 mmol) and Xantphos (1.02 g, 1.76 mmol). The resulting mixture was stirred for 3 h at 80° C. and was then concentrated under reduced pressure. Purification of the residue by normal phase chromatography afforded benzyl 4-(5-((6³S,4S)-2⁵-((diphenylmethylene)amino)-1¹-ethyl-10,10-dimethyl-5,7-dioxo-4-(((2-(trimethylsilyl)ethoxy)carbonyl)amino)-6¹,6²,6³,6⁴,6⁵,6⁶-hexahydro-1¹H-8-oxa-1(5,3)-indola-6(1,3)-pyridazina-2(1,3)-benzenacycloundecaphane-1²-yl)-6-((S)-1-methoxyethyl)pyridin-3-yl)piperazine-1-carboxylate (1.76 g, 1.51 mmol 12% yield over 3 steps) as a yellow solid. LCMS (ESI) m/z: [M+H] calcd for C₆₈H₈₀N₈O₈Si: 1165.6; found 1165.5.

Step 6. To a solution of benzyl 4-(5-((6³S,4S)-2⁵-((diphenylmethylene)amino)-1¹-ethyl-10,10-dimethyl-5,7-dioxo-4-(((2-(trimethylsilyl)ethoxy)carbonyl)amino)-6¹,6²,6³,6⁴,6⁵,6⁶-hexahydro-1 ¹H-8-oxa-1(5,3)-indola-6(1,3)-pyridazina-2(1,3)-benzenacycloundecaphane-1²-yl)-6-((S)-1-methoxyethyl)pyridin-3-yl)piperazine-1-carboxylate (1.76 g, 1.51 mmol) in THE (9.0 mL) at 0° C. was added a solution of citric acid (1.72 g, 8.95 mmol) in H₂O (9 mL). The resulting mixture was stirred for 2 h at room temperature and was then concentrated under reduced pressure. The residue was extracted with ethyl acetate (2×20 mL) and the combined organic extracts were washed with brine (3×10 mL), dried over Na₂SO₄, filtered, and concentrated under reduced pressure. Purification of the residue by normal phase chromatography afforded benzyl 4-(5-((6³S,4S)-2⁵-amino-1′-ethyl-10,10-dimethyl-5,7-dioxo-4-(((2-(trimethylsilyl)ethoxy)carbonyl)amino)-6¹,6²,6³,6⁴,6⁵,6⁶-hexahydro-1¹H-8-oxa-1(5,3)-indola-6(1,3)-pyridazina-2(1,3)-benzenacycloundecaphane-12-yl)-6-((S)-1-methoxyethyl)pyridin-3-yl)piperazine-1-carboxylate (1.22 g, 73% yield) as a yellow solid. LCMS (ESI) m/z: [M+H] calcd for C₅₅H₇₂N₈O₈Si: 1001.5; found 1001.6.

Step 7. To a solution of benzyl 4-(5-((6³S,4S)-2⁵-amino-1′-ethyl-10,10-dimethyl-5,7-dioxo-4-(((2-(trimethylsilyl)ethoxy)carbonyl)amino)-6¹,6²,6³,6⁴,6⁵,6⁶-hexahydro-1¹H-8-oxa-1(5,3)-indola-6(1,3)-pyridazina-2(1,3)-benzenacycloundecaphane-12-yl)-6-((S)-1-methoxyethyl)pyridin-3-yl)piperazine-1-carboxylate (1.10 g, 1.11 mmol) and tert-butyl (2S,3S)-2-formyl-3-(methyl((S)-3-methyl-1-oxo-1-(2-(trimethylsilyl)ethoxy)butan-2-yl)carbamoyl)pyrrolidine-1-carboxylate (1.01 g, 2.22 mmol) in MeOH (10 mL) at 0° C. were added ZnCl₂ (300 mg, 2.22 mmol) and NaBH₃CN (140 mg, 2.22 mmol). The resulting mixture was stirred for 2 h at room temperature and was then quenched at 0° C. by the addition of H₂O, filtered, and the filter cake washed with MeOH (3×30 mL). The filtrate was concentrated under reduced pressure, extracted with EtOAc (3×50 mL), dried over Na₂SO₄, filtered, and concentrated under reduced pressure. Purification by normal phase chromatography afforded benzyl 4-(5-((6³S,4S)-25-((((2S,3S)-1-(tert-butoxycarbonyl)-3-(methyl((S)-3-methyl-1-oxo-1-(2-(trimethylsilyl)ethoxy)butan-2-yl)carbamoyl)pyrrolidin-2-yl)methyl)amino)-1¹-ethyl-10,10-dimethyl-5,7-dioxo-4-(((2-(trimethylsilyl)ethoxy)carbonyl)amino)-6¹,6²,6³,6⁴,6⁵,6⁶-hexahydro-1¹H-8-oxa-1(5,3)-indola-6(1,3)-pyridazina-2(1,3)-benzenacycloundecaphane-12-yl)-6-((S)-1-methoxyethyl)pyridin-3-yl)piperazine-1-carboxylate (1.4 g, 71% yield) as a yellow solid. LCMS (ESI) m/z: [M+H] calcd for C₇₇H₁₁₂N₁₀O₁₃Si₂: 1441.8; found 1441.9.

Step 8. To a solution of benzyl 4-(5-((6³S,4S)-25-((((2S,3S)-1-(tert-butoxycarbonyl)-3-(methyl((S)-3-methyl-1-oxo-1-(2-(trimethylsilyl)ethoxy)butan-2-yl)carbamoyl)pyrrolidin-2-yl)methyl)amino)-1¹-ethyl-10,10-dimethyl-5,7-dioxo-4-(((2-(trimethylsilyl)ethoxy)carbonyl)amino)-6¹,6²,6³,6⁴,6⁵,6⁶-hexahydro-1¹H-8-oxa-1(5,3)-indola-6(1,3)-pyridazina-2(1,3)-benzenacycloundecaphane-12-yl)-6-((S)-1-methoxyethyl)pyridin-3-yl)piperazine-1-carboxylate (1.4 g, 0.97 mmol) in DMF (14 mL) at 0° C. was added CsF (1.47 g, 9.68 mmol). The resulting mixture was stirred overnight at room temperature, filtered, and the filter cake washed with EtOAc (3×30 mL). The filtrate was concentrated under reduced pressure to afford N-((2S,3S)-2-((((6³S,4S)-4-amino-1²-(5-(4-((benzyloxy)carbonyl)piperazin-1-yl)-2-((S)-1-methoxyethyl)pyridin-3-yl)-1¹-ethyl-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-2⁵-yl)amino)methyl)-1-(tert-butoxycarbonyl)pyrrolidine-3-carbonyl)-N-methyl-L-valine (1.61 g, crude) which taken directly to the next reaction without further purification. LCMS (ESI) m/z: [M/2+H] calcd for C₆₆H₈₈N₁₀O₁₁: 599.3; found 599.8.

Step 9. To a solution of N-((2S,3S)-2-((((6³S,4S)-4-amino-1²-(5-(4-((benzyloxy)carbonyl)piperazin-1-yl)-2-((S)-1-methoxyethyl)pyridin-3-yl)-1¹-ethyl-10,10-dimethyl-5,7-dioxo-6¹,6²,6³,6⁴,6⁵,6⁶-hexahydro-1 ¹H-8-oxa-1(5,3)-indola-6(1,3)-pyridazina-2(1,3)-benzenacycloundecaphane-2⁵-yl)amino)methyl)-1-(tert-butoxycarbonyl)pyrrolidine-3-carbonyl)-N-methyl-L-valine (1.61 g, crude) and DIPEA (1.74 g, 13.4 mmol) in DMF (161 mL) at 0° C. were added HOAt (275 mg, 2.02 mmol) and HATU (767 mg, 2.02 mmol). The resulting mixture was stirred for 4 h at room temperature and was then quenched at 0° C. by the addition of H₂O. The aqueous mixture was extracted with EtOAc (3×50 mL), and the combined organic extracts were washed with brine (3×100 mL), dried over Na₂SO₄, filtered, and concentrated under reduced pressure. Purification by normal phase chromatography afforded tert-butyl (3aS,6S,9S,15S,32aS)-21-(5-(4-((benzyloxy)carbonyl)piperazin-1-yl)-2-((S)-1-methoxyethyl)pyridin-3-yl)-22-ethyl-6-isopropyl-5,19,19-trimethyl-4,7,10,16-tetraoxo-2,3,3a,5,6,7,8,9,10,13,14,15,16,18,19,20,22,31,32,32a-icosahydro-12H-11,15-epimino-23,25-etheno-9,28-methano-26,30-(metheno)dipyrrolo[3,2-o:3′,4′-z][1]oxa[7,10,13,18]tetraazacyclotriacontine-1(4H)-carboxylate (450 mg, 39% yield over 2 steps) as a light yellow solid. LCMS (ESI) m/z: [M/2+H] calcd for C₆₆H₈₆N₁₀O₁₀: 590.3; found 590.5.

Step 10. To a solution of tert-butyl (3aS,6S,9S,15S,32aS)-21-(5-(4-((benzyloxy)carbonyl)piperazin-1-yl)-2-((S)-1-methoxyethyl)pyridin-3-yl)-22-ethyl-6-isopropyl-5,19,19-trimethyl-4,7,10,16-tetraoxo-2,3,3a,5,6,7,8,9,10,13,14,15,16,18,19,20,22,31,32,32a-icosahydro-12H-11,15-epimino-23,25-etheno-9,28-methano-26,30-(metheno)dipyrrolo[3,2-o:3′,4′-z][1]oxa[7,10,13,18]tetraazacyclotriacontine-1(4H)-carboxylate (450 mg, 0.382 mmol) and Et₃N (210 μL, 1.53 mmol) in DCM (4.5 mL) at 0° C. were added TFAA (160 μL, 1.15 mmol) and DMAP (4.66 mg, 0.038 mmol). The resulting mixture was stirred for 1.5 h at room temperature and was then quenched at 0° C. by the addition of sat. aq. NaHCO₃. The aqueous layer was extracted with DCM (3×20 mL) and the combined organic extracts were dried over Na₂SO₄, filtered, and concentrated under reduced pressure. Purification by normal phase chromatography afforded tert-butyl (3aS,6S,9S,15S,32aS)-21-(5-(4-((benzyloxy)carbonyl)piperazin-1-yl)-2-((S)-1-methoxyethyl)pyridin-3-yl)-22-ethyl-6-isopropyl-5,19,19-trimethyl-4,7,10,16-tetraoxo-8,31-bis(2,2,2-trifluoroacetyl)-2,3,3a,5,6,7,8,9,10,13,14,15,16,18,19,20,22,31,32,32a-icosahydro-12H-11,15-epimino-23,25-etheno-9,28-methano-26,30-(metheno)dipyrrolo[3,2-o:3′,4′-z][1]oxa[7,10,13,18]tetraazacyclotriacontine-1(4H)-carboxylate (450 mg, 82% yield) as a yellow solid. LCMS (ESI) m/z: [M+NH₄] calcd for C₇₀H₈₄F₆N₁₀O₁₂: 1388.7; found 1388.6.

Step 11. To a solution of tert-butyl (3aS,6S,9S,15S,32aS)-21-(5-(4-((benzyloxy)carbonyl)piperazin-1-yl)-2-((S)-1-methoxyethyl)pyridin-3-yl)-22-ethyl-6-isopropyl-5,19,19-trimethyl-4,7,10,16-tetraoxo-8,31-bis(2,2,2-trifluoroacetyl)-2,3,3a,5,6,7,8,9,10,13,14,15,16,18,19,20,22,31,32,32a-icosahydro-12H-11,15-epimino-23,25-etheno-9,28-methano-26,30-(metheno)dipyrrolo[3,2-o:3′,4′-z][1]oxa[7,10,13,18]tetraazacyclotriacontine-1(4H)-carboxylate (450 mg, 0.353 mmol) in IPA (4.5 mL) was added 20 wt. % Pd(OH)₂/C (450 mg). The resulting mixture was stirred overnight under an atmosphere of H₂, filtered, and concentrated under reduced pressure to afford tert-butyl (3aS,6S,9S,15S,32aS)-22-ethyl-6-isopropyl-21-(2-((S)-1-methoxyethyl)-5-(piperazin-1-yl)pyridin-3-yl)-5,19,19-trimethyl-4,7,10,16-tetraoxo-8,31-bis(2,2,2-trifluoroacetyl)-2,3,3a,5,6,7,8,9,10,13,14,15,16,18,19,20,22,31,32,32a-icosahydro-12H-11,15-epimino-23,25-etheno-9,28-methano-26,30-(metheno)dipyrrolo[3,2-o:3′,4′-z][1]oxa[7,10,13,18]tetraazacyclotriacontine-1(4H)-carboxylate (362 mg, crude) which was taken to the next reaction without further purification. LCMS (ESI) m/z: [M+Na] calcd for C₆₂H₇₈F₆N₁₀O₁₀: 1259.6; found 1259.6.

Step 12. To a solution of tert-butyl (3aS,6S,9S,15S,32aS)-22-ethyl-6-isopropyl-21-(2-((S)-1-methoxyethyl)-5-(piperazin-1-yl)pyridin-3-yl)-5,19,19-trimethyl-4,7,10,16-tetraoxo-8,31-bis(2,2,2-trifluoroacetyl)-2,3,3a,5,6,7,8,9,10,13,14,15,16,18,19,20,22,31,32,32a-icosahydro-12H-11,15-epimino-23,25-etheno-9,28-methano-26,30-(metheno)dipyrrolo[3,2-o:3′,4′-z][1]oxa[7,10,13,18]tetraazacyclotriacontine-1(4H)-carboxylate (350 mg, crude) and acetic acid (50 μL, 0.921 mmol) in IPA (3.5 mL) at 0° C. were added (1-ethoxycyclopropoxy)trimethylsilane (160 mg, 0.921 mmol) and NaBH₃CN (58 mg, 0.92 mmol). The resulting mixture was stirred for 2 h at 60° C. and was then quenched at 0° C. by the addition of H₂O. The aqueous mixture was extracted with EtOAc (3×10 mL), and the combined organic extracts were washed with sat. aq. NaHCO₃ (3×20 mL), dried over Na₂SO₄, filtered, and concentrated under reduced pressure. Purification by normal phase chromatography afforded tert-butyl (3aS,6S,9S,15S,32aS)-21-(5-(4-cyclopropylpiperazin-1-yl)-2-((S)-1-methoxyethyl)pyridin-3-yl)-22-ethyl-6-isopropyl-5,19,19-trimethyl-4,7,10,16-tetraoxo-8,31-bis(2,2,2-trifluoroacetyl)-2,3,3a,5,6,7,8,9,10,13,14,15,16,18,19,20,22,31,32,32a-icosahydro-12H-11,15-epimino-23,25-etheno-9,28-methano-26,30-(metheno)dipyrrolo[3,2-o:3′,4′-z][1]oxa[7,10,13,18]tetraazacyclotriacontine-1(4H)-carboxylate (273 mg, 0.214 mmol 63% yield over 2 steps) as a yellow solid. LCMS (ESI) m/z: [M+H] calcd for C₆₅H₈₂F₆N₁₀O₁₀: 1277.6; found 1277.7.

Step 13. To a solution of tert-butyl (3aS,6S,9S,15S,32aS)-21-(5-(4-cyclopropylpiperazin-1-yl)-2-((S)-1-methoxyethyl)pyridin-3-yl)-22-ethyl-6-isopropyl-5,19,19-trimethyl-4,7,10,16-tetraoxo-8,31-bis(2,2,2-trifluoroacetyl)-2,3,3a,5,6,7,8,9,10,13,14,15,16,18,19,20,22,31,32,32a-icosahydro-12H-11,15-epimino-23,25-etheno-9,28-methano-26,30-(metheno)dipyrrolo[3,2-o:3′,4′-z][1]oxa[7,10,13,18]tetraazacyclotriacontine-1(4H)-carboxylate (265 mg, 0.224 mmol) in DCM (10 mL) at 0° C. was added TFA (2 mL). The resulting mixture was stirred for 1.5 h at room temperature and was then basified to pH=8 with sat. aq. NaHCO₃. The aqueous mixture was extracted with DCM (3×10 mL) and the combined organic extracts were dried over Na₂SO₄, filtered, and concentrated under reduced pressure to afford (3aS,6S,9S,15S,32aS)-21-(5-(4-cyclopropylpiperazin-1-yl)-2-((S)-1-methoxyethyl)pyridin-3-yl)-22-ethyl-6-isopropyl-5,19,19-trimethyl-8,31-bis(2,2,2-trifluoroacetyl)-2,3,3a,5,6,8,9,14,15,18,19,20,22,31,32,32a-hexadecahydro-12H-11,15-epimino-23,25-etheno-9,28-methano-26,30-(metheno)dipyrrolo[3,2-o:3′,4′-z][1]oxa[7,10,13,18]tetraazacyclotriacontine-4,7,10,16(1H,13H)-tetraone (223 mg, crude) which was taken to the next reaction without further purification. LCMS (ESI) m/z: [M+H] calcd for C₆₀H₇₄F₆N₁₀O₈: 1177.6; found 1177.6.

Step 14. To a solution of (3aS,6S,9S,15S,32aS)-21-(5-(4-cyclopropylpiperazin-1-yl)-2-((S)-1-methoxyethyl)pyridin-3-yl)-22-ethyl-6-isopropyl-5,19,19-trimethyl-8,31-bis(2,2,2-trifluoroacetyl)-2,3,3a,5,6,8,9,14,15,18,19,20,22,31,32,32a-hexadecahydro-12H-11,15-epimino-23,25-etheno-9,28-methano-26,30-(metheno)dipyrrolo[3,2-o:3′,4′-z][1]oxa[7,10,13,18]tetraazacyclotriacontine-4,7,10,16(1H,13H)-tetraone (220 mg, crude) and DIPEA (330 μL, 1.87 mmol) in DMF (3.0 mL) at 0° C. were added 2-fluoro-2-methylpropanoic acid (39.7 mg, 0.374 mmol) and COMU (120 mg, 0.280 mmol). The resulting mixture was stirred for 1 h at 0° C. and was then quenched by the addition of cold H₂O. The aqueous mixture was extracted with EtOAc (3×20 mL), and the combined organic extracts were washed with brine (3×10 mL), dried over Na₂SO₄, filtered, and concentrated under reduced pressure. Purification by normal phase prep-TLC afforded (3aS,6S,9S,15S,32aS)-21-(5-(4-cyclopropylpiperazin-1-yl)-2-((S)-1-methoxyethyl)pyridin-3-yl)-22-ethyl-1-(2-fluoro-2-methylpropanoyl)-6-isopropyl-5,19,19-trimethyl-8,31-bis(2,2,2-trifluoroacetyl)-2,3,3a,5,6,8,9,14,15,18,19,20,22,31,32,32a-hexadecahydro-12H-11,15-epimino-23,25-etheno-9,28-methano-26,30-(metheno)dipyrrolo[3,2-o:3′,4′-z][1]oxa[7,10,13,18]tetraazacyclotriacontine-4,7,10,16(1H,13H)-tetraone (128 mg, 46% yield over 2 steps) as a yellow solid. LCMS (ESI) m/z: [M+H] calcd for C₆₄H₇₉F₇N₁₀O₉: 1265.6; found 1265.5.

Step 15. To a solution of (3aS,6S,9S,15S,32aS)-21-(5-(4-cyclopropylpiperazin-1-yl)-2-((S)-1-methoxyethyl)pyridin-3-yl)-22-ethyl-1-(2-fluoro-2-methylpropanoyl)-6-isopropyl-5,19,19-trimethyl-8,31-bis(2,2,2-trifluoroacetyl)-2,3,3a,5,6,8,9,14,15,18,19,20,22,31,32,32a-hexadecahydro-12H-11,15-epimino-23,25-etheno-9,28-methano-26,30-(metheno)dipyrrolo[3,2-o:3′,4′-z][1]oxa[7,10,13,18]tetraazacyclotriacontine-4,7,10,16(1H,13H)-tetraone (120 mg, 0.095 mmol) in MeOH (1.5 mL) at 0° C. was added a solution of K₂CO₃ (52.4 mg, 0.380 mmol) in H₂O (1.5 mL). The resulting mixture was stirred overnight at room temperature and was then acidified to pH=6 with 1 N aq. HCl. The aqueous mixture was extracted with DCM (3×10 mL) and the combined organic extracts were dried over Na₂SO₄, filtered, and concentrated under reduced pressure to afford (S)-1-((1²S,1³S,6S,9S)-4⁵-(2-(5-(4-cyclopropylpiperazin-1-yl)-2-((S)-1-methoxyethyl)pyridin-3-yl)-1-ethyl-3-(3-hydroxy-2,2-dimethylpropyl)-1H-indol-5-yl)-1¹-(2-fluoro-2-methylpropanoyl)-9-isopropyl-10-methyl-8,11-dioxo-3,7,10-triaza-1(2,3)-pyrrolidina-4(1,3)-benzenacycloundecaphane-6-carbonyl)hexahydropyridazine-3-carboxylic acid (109 mg, crude) which was taken to the next reaction without further purification. LCMS (ESI) m/z: [M+H] calcd for C₆₀H₈₃FN₁₀O₈: 1091.7; found 1091.8.

Step 16. To a solution of (S)-1-((1²S,1³S,6S,9S)-4⁵-(2-(5-(4-cyclopropylpiperazin-1-yl)-2-((S)-1-methoxyethyl)pyridin-3-yl)-1-ethyl-3-(3-hydroxy-2,2-dimethylpropyl)-1H-indol-5-yl)-1¹-(2-fluoro-2-methylpropanoyl)-9-isopropyl-10-methyl-8,11-dioxo-3,7,10-triaza-1(2,3)-pyrrolidina-4(1,3)-benzenacycloundecaphane-6-carbonyl)hexahydropyridazine-3-carboxylic acid (104 mg, crude) and DIPEA (500 μL, 2.85 mmol) in DCM (10 mL) at 0° C. were added EDCI (364 mg, 1.90 mmol) and HOBT (128 mg, 0.950 mmol). The resulting mixture was stirred overnight at room temperature and was then neutralized with 1M aq. HCl. The aqueous mixture was extracted with DCM (3×20 mL) and the combined organic extracts were dried over Na₂SO₄, filtered, and concentrated under reduced pressure. Purification by reversed phase chromatography afforded (3aS,6S,9S,15S,32aS)-21-(5-(4-cyclopropylpiperazin-1-yl)-2-((S)-1-methoxyethyl)pyridin-3-yl)-22-ethyl-1-(2-fluoro-2-methylpropanoyl)-6-isopropyl-5,19,19-trimethyl-2,3,3a,5,6,8,9,14,15,18,19,20,22,31,32,32a-hexadecahydro-12H-11,15-epimino-23,25-etheno-9,28-methano-26,30-(metheno)dipyrrolo[3,2-o:3′,4′-z][1]oxa[7,10,13,18]tetraazacyclotriacontine-4,7,10,16(1H,13H)-tetraone (16.4 mg, 17% yield over 2 steps) as a white solid. LCMS (ESI) m/z: [M+H] calcd for C₆₀H₈₁FN₁₀O₇: 1073.6; found 1073.5. ¹H NMR (400 MHz, DMSO-d₆) δ 8.52 (d, J=3.0 Hz, 1H), 7.97 (d, J=7.0 Hz, 2H), 7.74-7.53 (m, 2H), 7.43 (s, 1H), 7.05-6.97 (m, 2H), 6.34-6.29 (m, 1H), 5.31-5.23 (m, 2H), 4.69 (d, J=11.1 Hz, 1H), 4.62-4.41 (m, 1H), 4.31-4.12 (m, 3H), 4.11-4.01 (m, 3H), 3.72-3.66 (m, 5H), 3.30-3.23 (m, 2H), 3.10 (s, 3H), 2.95-2.89 (m, 2H), 2.82-2.63 (m, 7H), 2.40 (s, 3H), 2.29-2.20 (m, 2H), 2.07-1.96 (m, 3H), 1.87-1.82 (m, 1H), 1.77-1.47 (m, 10H), 1.42-1.31 (m, 4H), 1.33-1.19 (m, 1H), 1.00-0.47 (m, 20H).

### Example A365. Synthesis of (3aS,6S,9S,15S,32aS)-22-ethyl-1-(2-fluoro-2-methylpropanoyl)-6-isopropyl-21-(2-((S)-1-methoxyethyl)-5-(pyridin-3-yloxy)pyridin-3-yl)-5,19,19-trimethyl-3,3a,5,6,8,9,14,15,18,19,20,22,32,32a-tetradecahydro-1H,12H-11,15-epimino-23,25-etheno-9,28-methano-26,30-(metheno)dipyrrolo[2,3-c:3′,4′-v][1,18]dioxa[6,9,12]triazacyclotriacontine-4,7,10,16(2H,13H)-tetraone

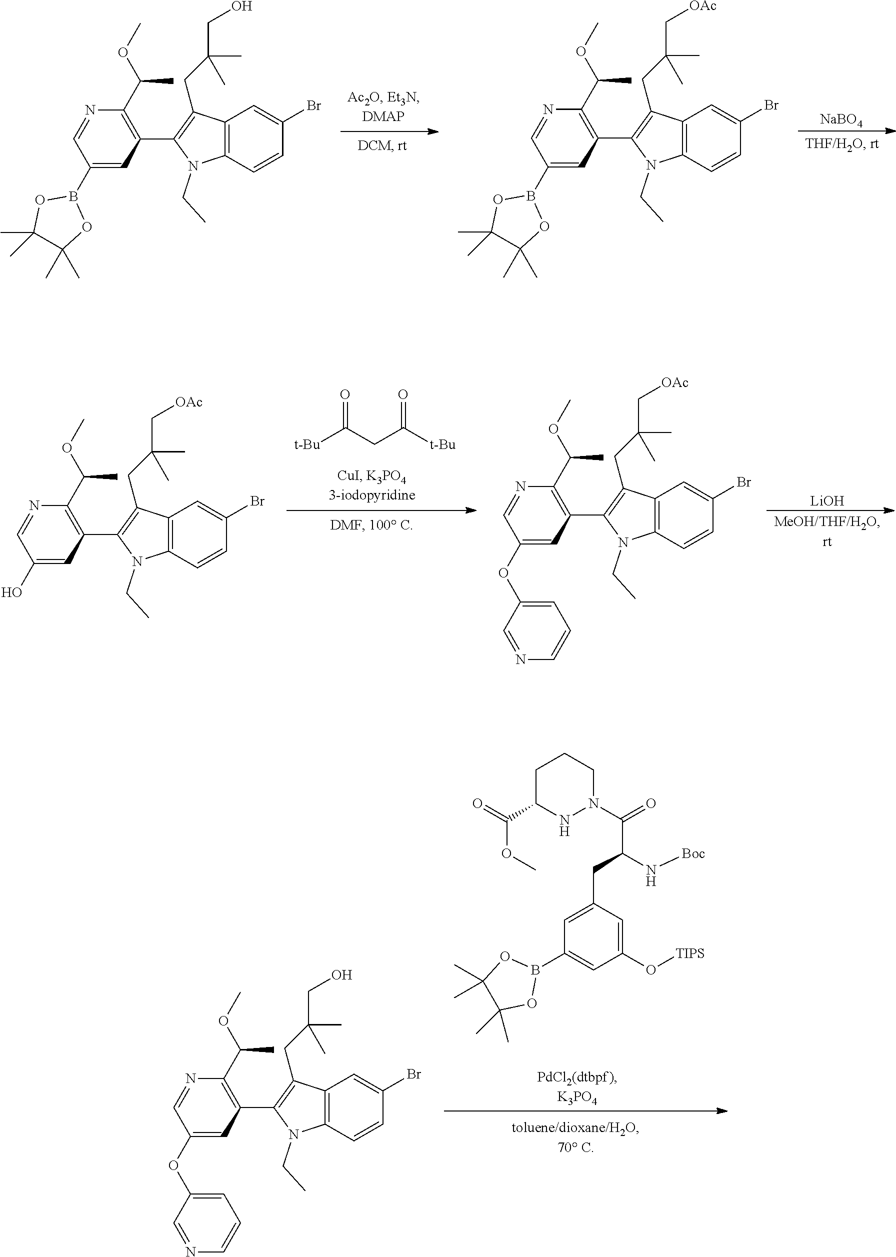

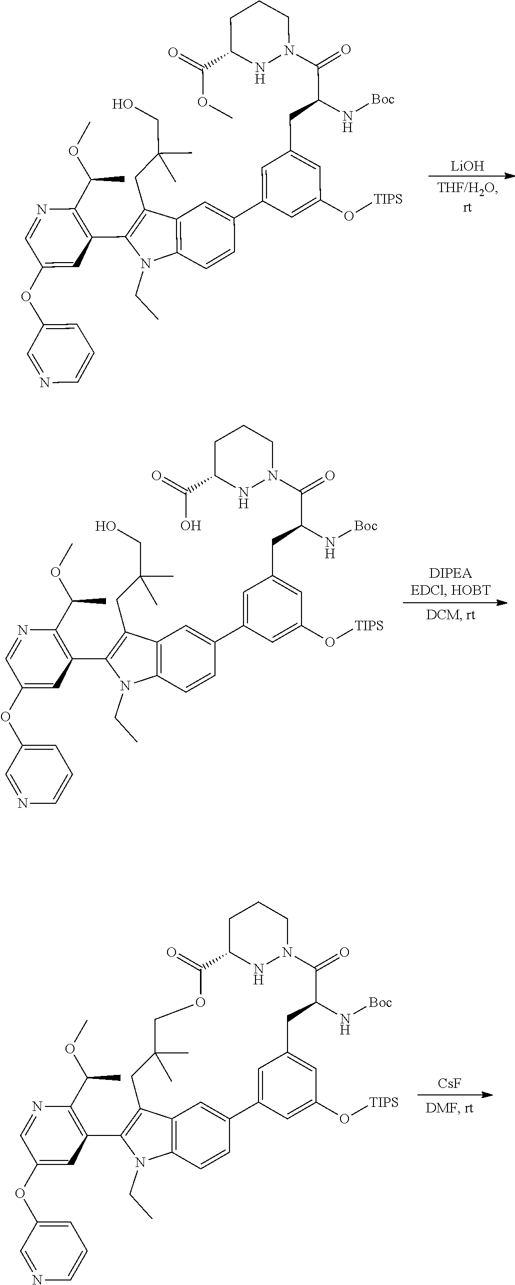

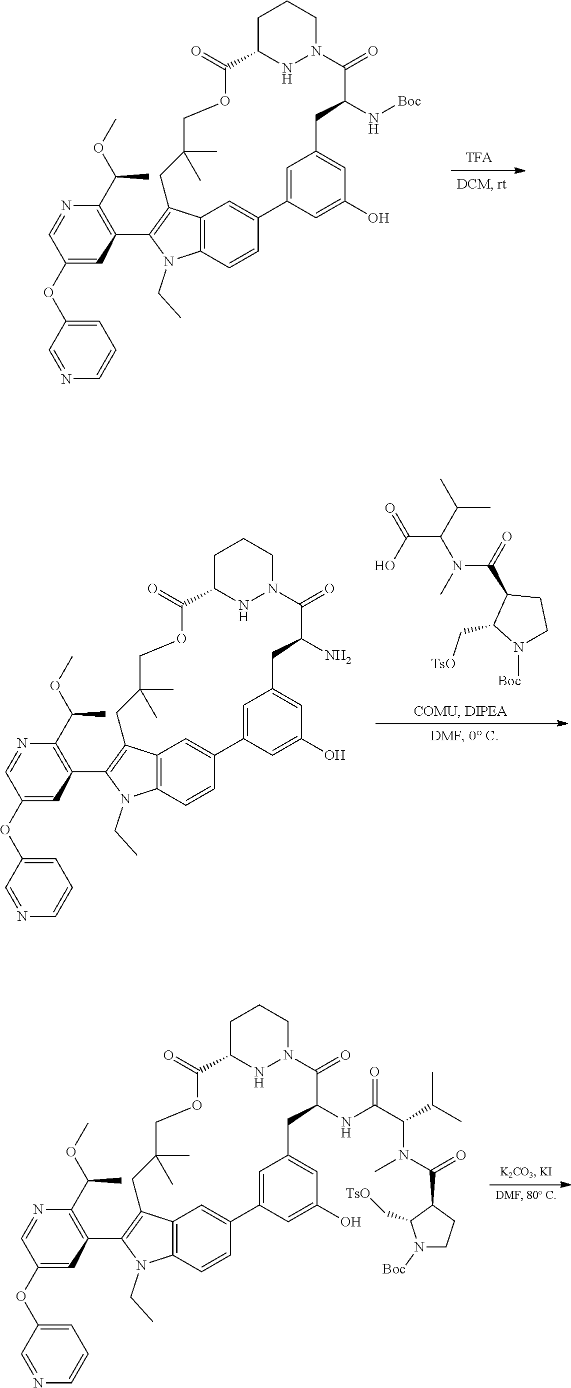

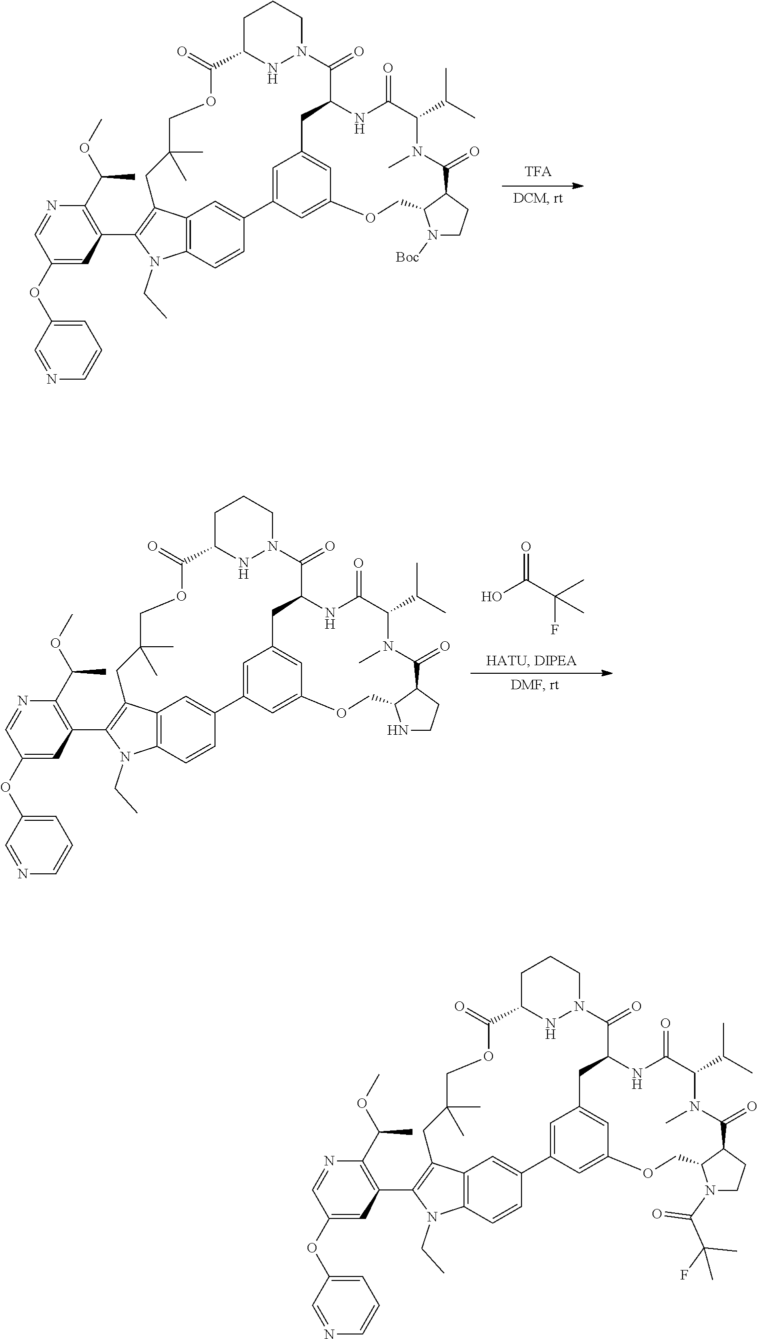

Step 1. To a solution of (S)-3-(5-bromo-1-ethyl-2-(2-(1-methoxyethyl)-5-(4,4,5,5-tetramethyl-1,3,2-dioxaborolan-2-yl)pyridin-3-yl)-1H-indol-3-yl)-2,2-dimethylpropan-1-ol (30 g, 52.5 mmol) and Et₃N (15.9 g, 157 mmol) in DCM (300 mL) at 0° C. were added DMAP (641 mg, 5.25 mmol) and Ac₂O (6.43 g, 63.0 mmol). The resulting mixture was stirred for 1 h at room temperature and concentrated under reduced pressure. The concentrate was diluted by the addition of H₂O (100 mL), acidified to pH=5 by the addition of 1 M aq. HCl, and extracted with EtOAc (3×100 mL). The combined organic extracts were dried over Na₂SO₄, filtered, and concentrated under reduced pressure to afford (S)-3-(5-bromo-1-ethyl-2-(2-(1-methoxyethyl)-5-(4,4,5,5-tetramethyl-1,3,2-dioxaborolan-2-yl)pyridin-3-yl)-1H-indol-3-yl)-2,2-dimethylpropyl acetate (34.5 g, crude) which was used in the next reaction without further purification. LCMS (ESI) m/z: [M+H] calcd for C₃₁H₄₂BBrN₂O₅: 613.3; found 613.4.

Step 2. To a solution of (S)-3-(5-bromo-1-ethyl-2-(2-(1-methoxyethyl)-5-(4,4,5,5-tetramethyl-1,3,2-dioxaborolan-2-yl)pyridin-3-yl)-1H-indol-3-yl)-2,2-dimethylpropyl acetate (34.4 g, crude) in THE (300 mL) at 0° C. was added a solution of sodium perborate (13.8 g, 168 mmol) in H₂O (100 mL). The resulting mixture was stirred overnight at room temperature. The aqueous mixture was extracted with EtOAc (3×100 mL), and the combined organic extracts were dried over Na₂SO₄, filtered, and concentrated under reduced pressure. Purification by normal phase chromatography afforded (S)-3-(5-bromo-1-ethyl-2-(5-hydroxy-2-(1-methoxyethyl)pyridin-3-yl)-1H-indol-3-yl)-2,2-dimethylpropyl acetate (26.8 g, 95% yield) as a grey solid. LCMS (ESI) m/z: [M+H] calcd for C₂₅H₃₁BrN₂O₄: 503.2; found 503.2.

Step 3. To a solution of (S)-3-(5-bromo-1-ethyl-2-(5-hydroxy-2-(1-methoxyethyl)pyridin-3-yl)-1H-indol-3-yl)-2,2-dimethylpropyl acetate (3.0 g, 5.9 mmol) in DMF (30 mL) were added 2,2,6,6-tetramethyl-3,5-heptanedione (439 mg, 2.38 mmol), 3-iodopyridine (1.83 g, 8.93 mmol), CuI (113 mg, 0.596 mmol) and K₃PO₄ (2.53 g, 11.9 mmol). The resulting mixture was stirred for 5 h at 100° C. and quenched at 0° C. by the addition of H₂O. The aqueous mixture was extracted with EtOAc (2×50 mL), and the combined organic extracts were washed with brine (3×100 mL), dried over Na₂SO₄, filtered, and concentrated under reduced pressure. Purification by reversed phase chromatography afforded (S)-3-(5-bromo-1-ethyl-2-(2-(1-methoxyethyl)-5-(pyridin-3-yloxy)pyridin-3-yl)-1H-indol-3-yl)-2,2-dimethylpropyl acetate (1.52 g, 44% yield) as an off-white solid. LCMS (ESI) m/z: [M+H] calcd for C₃₀H₃₄BrN₃O₄: 580.2; found 580.1.

Step 4. To a solution of (S)-3-(5-bromo-1-ethyl-2-(2-(1-methoxyethyl)-5-(pyridin-3-yloxy)pyridin-3-yl)-1H-indol-3-yl)-2,2-dimethylpropyl acetate (1.60 g, 2.76 mmol) in THE (9.0 mL), MeOH (3.0 mL), and H₂O (3.0 mL) at 0° C. was added LiOH·H₂O (347 mg, 8.29 mmol). The resulting mixture was stirred for 2 h at room temperature and concentrated under reduced pressure. The concentrate was extracted with 10:1 vol. DCM/MeOH (3×30 mL), and the combined organic extracts were dried over Na₂SO₄, filtered, and concentrated under reduced pressure to afford (S)-3-(5-bromo-1-ethyl-2-(2-(1-methoxyethyl)-5-(pyridin-3-yloxy)pyridin-3-yl)-1H-indol-3-yl)-2,2-dimethylpropan-1-ol (1.47 g, crude) which was taken to the next reaction without further purification. LCMS (ESI) m/z: [M+H] calcd for C₂₈H₃₂BrN₃O₃: 538.2; found 538.1.

Step 5. To a solution of (S)-3-(5-bromo-1-ethyl-2-(2-(1-methoxyethyl)-5-(pyridin-3-yloxy)pyridin-3-yl)-1H-indol-3-yl)-2,2-dimethylpropan-1-ol (1.45 g, 2.70 mmol) and methyl (S)-1-((S)-2-((tert-butoxycarbonyl)amino)-3-(3-(4,4,5,5-tetramethyl-1,3,2-dioxaborolan-2-yl)-5-((triisopropylsilyl)oxy)phenyl)propanoyl)hexahydropyridazine-3-carboxylate (2.23 g, crude) in toluene (15 mL), dioxane (5.0 mL) and H₂O (5.0 mL) were added K₃PO₄ (1.71 g, 8.08 mmol) and Pd(dppf)Cl₂ (197 mg, 0.269 mmol). The resulting mixture was stirred for 3 h at 70° C. and quenched at room temperature by the addition of H₂O. The aqueous mixture was extracted with EtOAc (2×50 mL), and the combined organic extracts were dried over Na₂SO₄, filtered, and concentrated under reduced pressure to afford methyl (S)-1-((S)-2-((tert-butoxycarbonyl)amino)-3-(3-(1-ethyl-3-(3-hydroxy-2,2-dimethylpropyl)-2-(2-((S)-1-methoxyethyl)-5-(pyridin-3-yloxy)pyridin-3-yl)-1H-indol-5-yl)-5-((triisopropylsilyl)oxy)phenyl)propanoyl)hexahydropyridazine-3-carboxylate (3.83 g, crude) which was used in the next reaction without further purification. LCMS (ESI) m/z: [M+H] calcd for C₅₇H₈₀N₆O₉Si: 1021.6; found 1021.5.

Step 6. To a solution of methyl (S)-1-((S)-2-((tert-butoxycarbonyl)amino)-3-(3-(1-ethyl-3-(3-hydroxy-2,2-dimethylpropyl)-2-(2-((S)-1-methoxyethyl)-5-(pyridin-3-yloxy)pyridin-3-yl)-1H-indol-5-yl)-5-((triisopropylsilyl)oxy)phenyl)propanoyl)hexahydropyridazine-3-carboxylate (3.71 g, crude) in THE (20 mL) and H₂O (20 mL) at 0° C. was added a solution of LiOH·H₂O (167 mg, 3.40 mmol) in H₂O (10 mL). The resulting mixture was stirred for 3 h at room temperature, concentrated under reduced pressure, and the concentrate acidified to pH=5 by the addition of 1M aq. HCl. The aqueous mixture was extracted with 3:1 vol. DCM/IPA (3×50 mL), and the combined organic extracts were dried over Na₂SO₄, filtered, and concentrated under reduced pressure to afford (S)-1-((S)-2-((tert-butoxycarbonyl)amino)-3-(3-(1-ethyl-3-(3-hydroxy-2,2-dimethylpropyl)-2-(2-((S)-1-methoxyethyl)-5-(pyridin-3-yloxy)pyridin-3-yl)-1H-indol-5-yl)-5-((triisopropylsilyl)oxy)phenyl)propanoyl)hexahydropyridazine-3-carboxylic acid (3.78 g, crude) which was used in the next reaction without further purification. LCMS (ESI) m/z: [M+H] calcd for C₅₆H₇₈N₆O₉Si: 1007.6; found 1007.6.

Step 7. To a solution of (S)-1-((S)-2-((tert-butoxycarbonyl)amino)-3-(3-(1-ethyl-3-(3-hydroxy-2,2-dimethylpropyl)-2-(2-((S)-1-methoxyethyl)-5-(pyridin-3-yloxy)pyridin-3-yl)-1H-indol-5-yl)-5-((triisopropylsilyl)oxy)phenyl)propanoyl)hexahydropyridazine-3-carboxylic acid (3.78 g, crude) and DIPEA (19.5 g, 150 mmol) in DCM (380 mL) at 0° C. were added HOBT (5.10 g, 37.7 mmol) and EDCI (21.7 g, 113 mmol). The resulting mixture was stirred for 12 h at room temperature, diluted by the addition of H₂O (200 mL) and neutralized by the addition of 1M aq. HCl. The aqueous mixture was extracted with DCM (3×100 mL), and the combined organic extracts were washed with H₂O (3×400 mL), dried over Na₂SO₄, filtered, and concentrated under reduced pressure. Purification by normal phase chromatography afforded tert-butyl ((6³S,4S)-1¹-ethyl-1²-(2-((S)-1-methoxyethyl)-5-(pyridin-3-yloxy)pyridin-3-yl)-10,10-dimethyl-5,7-dioxo-2⁵-((triisopropylsilyl)oxy)-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)carbamate (1.84 g, 67% yield over 4 steps) as a yellow solid. LCMS (ESI) m/z: [M+H] calcd for C₅₆H₇₆N₆O₈Si: 989.6; found 989.5.

Step 8. To a solution of tert-butyl ((6³S,4S)-1¹-ethyl-1²-(2-((S)-1-methoxyethyl)-5-(pyridin-3-yloxy)pyridin-3-yl)-10,10-dimethyl-5,7-dioxo-2⁵-((triisopropylsilyl)oxy)-6¹,6²,6³,6⁴,6⁵,6⁶-hexahydro-1 ¹H-8-oxa-1(5,3)-indola-6(1,3)-pyridazina-2(1,3)-benzenacycloundecaphane-4-yl)carbamate (1.84 g, 1.86 mmol) in DMF (19 mL) stirred at 0° C. was added CsF (2.82 g, 18.6 mmol). The resulting mixture was stirred for 1 h at room temperature and quenched at 0° C. by the addition of H₂O. The aqueous mixture was extracted with EtOAc (2×60 mL), and the combined organic extracts were washed with brine (3×120 mL), dried over Na₂SO₄, filtered, and concentrated under reduced pressure. Purification by normal phase chromatography afforded tert-butyl ((6³S,4S)-1¹-ethyl-2⁵-hydroxy-1²-(2-((S)-1-methoxyethyl)-5-(pyridin-3-yloxy)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)carbamate (1.52 g, 98% yield) as an off-white solid. LCMS (ESI) m/z: [M+H] calcd for C₄₇H₅₆N₆O₈: 833.4; found 833.5.

Step 9. To a solution of tert-butyl ((6³S,4S)-1¹-ethyl-2⁵-hydroxy-1²-(2-((S)-1-methoxyethyl)-5-(pyridin-3-yloxy)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)carbamate (1.52 g, 1.83 mmol) in DCM (12 mL) at 0° C. was added TFA (4.0 mL). The resulting mixture was stirred for 1 h at room temperature, quenched at 0° C. by the addition of H₂O, and basified to pH=8 by the addition of sat. aq. NaHCO₃. The aqueous mixture was extracted with DCM (3×50 mL), and the combined organic extracts were dried with Na₂SO₄, filtered, and concentrated under reduced pressure to afford (6³S,4S)-4-amino-1′-ethyl-2⁵-hydroxy-1²-(2-((S)-1-methoxyethyl)-5-(pyridin-3-yloxy)pyridin-3-yl)-10,10-dimethyl-6¹,6²,6³,6⁴,6⁵,6⁶-hexahydro-1¹H-8-oxa-1(5,3)-indola-6(1,3)-pyridazina-2(1,3)-benzenacycloundecaphane-5,7-dione (1.28 g, crude) which was taken to the next reaction without further purification. LCMS (ESI) m/z: [M+H] calcd for C₄₂H₄₈N₆O₆: 733.4; found 733.5.

Step 10. To a solution of (6³S,4S)-4-amino-1′-ethyl-2⁵-hydroxy-1²-(2-((S)-1-methoxyethyl)-5-(pyridin-3-yloxy)pyridin-3-yl)-10,10-dimethyl-6¹,6²,6³,6⁴,6⁵,6⁶-hexahydro-1¹H-8-oxa-1(5,3)-indola-6(1,3)-pyridazina-2(1,3)-benzenacycloundecaphane-5,7-dione (1.28 g, crude) and N-((2S,3S)-1-(tert-butoxycarbonyl)-2-((tosyloxy)methyl)pyrrolidine-3-carbonyl)-N-methyl-L-valine (1.34 g, 2.62 mmol) in DMF (13 mL) at 0° C. were added DIPEA (9.03 g, 69.9 mmol) and COMU (1.05 g, 2.45 mmol). The resulting mixture was stirred for 1 h at 0° C. and quenched at −10° C. by the addition of H₂O. The aqueous mixture was extracted with EtOAc (2×50 mL), and the combined organic extracts were washed with brine (3×100 mL), dried over Na₂SO₄, filtered, and concentrated under reduced pressure. Purification by normal phase chromatography afforded tert-butyl (2S,3S)-3-(((2S)-1-(((6³S,4S)-1¹-ethyl-2⁵-hydroxy-1²-(2-((S)-1-methoxyethyl)-5-(pyridin-3-yloxy)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)amino)-3-methyl-1-oxobutan-2-yl)(methyl)carbamoyl)-2-((tosyloxy)methyl)pyrrolidine-1-carboxylate (1.92 g, 85% yield over 2 steps) as a brown solid. LCMS (ESI) m/z: [M+H] calcd for C₆₆H₈₂N₈O₁₃S: 1227.6; found 1227.6.

Step 11. To a solution of tert-butyl (2S,3S)-3-(((2S)-1-(((6³S,4S)-1¹-ethyl-2⁵-hydroxy-1²-(2-((S)-1-methoxyethyl)-5-(pyridin-3-yloxy)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)amino)-3-methyl-1-oxobutan-2-yl)(methyl)carbamoyl)-2-((tosyloxy)methyl)pyrrolidine-1-carboxylate (1.82 g, 1.48 mmol) in DMF (190 mL) were added K₂CO₃ (2.05 g, 14.8 mmol) and KI (246 mg, 1.48 mmol). The resulting mixture was stirred for 2 h at 80° C. and quenched at 0° C. by the addition of H₂O. The aqueous mixture was extracted with EtOAc (2×300 mL), and the combined organic extracts were washed with brine (3×600 mL), dried over Na₂SO₄, filtered, and concentrated under reduced pressure. Purification by normal phase chromatography afforded tert-butyl (3aS,6S,9S,15S,32aS)-22-ethyl-6-isopropyl-21-(2-((S)-1-methoxyethyl)-5-(pyridin-3-yloxy)pyridin-3-yl)-5,19,19-trimethyl-4,7,10,16-tetraoxo-2,3,3a,4,5,6,7,8,9,10,13,14,15,16,18,19,20,22,32,32a-icosahydro-1H,12H-11,15-epimino-23,25-etheno-9,28-methano-26,30-(metheno)dipyrrolo[2,3-c:3′,4′-v][1,18]dioxa[6,9,12]triazacyclotriacontine-1-carboxylate (753 mg, 48% yield) as an off-white solid. LCMS (ESI) m/z: [M+H] calcd for C₅₉H₇₄N₈O₁₀: 1055.6; found 1056.1.

Step 12. To a solution of tert-butyl (3aS,6S,9S,15S,32aS)-22-ethyl-6-isopropyl-21-(2-((S)-1-methoxyethyl)-5-(pyridin-3-yloxy)pyridin-3-yl)-5,19,19-trimethyl-4,7,10,16-tetraoxo-2,3,3a,4,5,6,7,8,9,10,13,14,15,16,18,19,20,22,32,32a-icosahydro-1H,12H-11,15-epimino-23,25-etheno-9,28-methano-26,30-(metheno)dipyrrolo[2,3-c:3′,4′-v][1,18]dioxa[6,9,12]triazacyclotriacontine-1-carboxylate (100 mg, 0.095 mmol) in DCM (2.0 mL) at 0° C. was added TFA (400 mL). The resulting mixture was stirred for 1 h at room temperature and basified to pH=8 by the addition of sat. aq. NaHCO₃. The aqueous mixture was extracted with DCM (3×20 mL) and the combined organic extracts were dried over Na₂SO₄, filtered, and concentrated under reduced pressure to afford (3aS,6S,9S,15S,32aS)-22-ethyl-6-isopropyl-21-(2-((S)-1-methoxyethyl)-5-(pyridin-3-yloxy)pyridin-3-yl)-5,19,19-trimethyl-3,3a,5,6,8,9,14,15,18,19,20,22,32,32a-tetradecahydro-1H,12H-11,15-epimino-23,25-etheno-9,28-methano-26,30-(metheno)dipyrrolo[2,3-c:3′,4′-v][1,18]dioxa[6,9,12]triazacyclotriacontine-4,7,10,16(2H,13H)-tetraone (117 mg, crude) which was taken to the next reaction without further purification. LCMS (ESI) m/z: [M+H] calcd for C₅₄H₆₆N₈O₈: 955.5; found 955.6.

Step 13. To a solution of (3aS,6S,9S,15S,32aS)-22-ethyl-6-isopropyl-21-(2-((S)-1-methoxyethyl)-5-(pyridin-3-yloxy)pyridin-3-yl)-5,19,19-trimethyl-3,3a,5,6,8,9,14,15,18,19,20,22,32,32a-tetradecahydro-1H,12H-11,15-epimino-23,25-etheno-9,28-methano-26,30-(metheno)dipyrrolo[2,3-c:3′,4′-v][1,18]dioxa[6,9,12]triazacyclotriacontine-4,7,10,16(2H,13H)-tetraone (117 mg, crude) and 2-fluoro-2-methylpropanoic acid (26.0 mg, crude) in DMF (2.0 mL) at 0° C. were added DIPEA (158 mg, 1.22 mmol) and HATU (69.9 mg, 0.183 mmol). The resulting mixture was stirred for 1 h at room temperature and quenched at 0° C. by the addition of H₂O. The aqueous mixture was extracted with EtOAc (2×20 mL), and the combined organic extracts were washed with brine (3×40 mL), dried over Na₂SO₄, filtered, and concentrated under reduced pressure. Purification by reversed phase chromatography afforded (3aS,6S,9S,15S,32aS)-22-ethyl-1-(2-fluoro-2-methylpropanoyl)-6-isopropyl-21-(2-((S)-1-methoxyethyl)-5-(pyridin-3-yloxy)pyridin-3-yl)-5,19,19-trimethyl-3,3a,5,6,8,9,14,15,18,19,20,22,32,32a-tetradecahydro-1H,12H-11,15-epimino-23,25-etheno-9,28-methano-26,30-(metheno)dipyrrolo[2,3-c:3′,4′-v][1,18]dioxa[6,9,12]triazacyclotriacontine-4,7,10,16(2H,13H)-tetraone (24.4 mg, 25% yield over 2 steps) as a white solid. LCMS (ESI) m/z: [M+H] calcd for C₅₈H₇₁FN₈O₉: 1043.5; found 1043.5; ¹H NMR (400 MHz, DMSO-d₆) δ 8.63-8.56 (m, 1H), 8.41 (d, J=2.8 Hz, 1H), 8.34 (d, J=4.5 Hz, 1H), 8.12-7.91 (m, 1H), 7.71-7.45 (m, 4H), 7.44-7.35 (m, 2H), 7.34-7.08 (m, 2H), 6.75-6.65 (m, 1H), 5.41-4.86 (m, 2H), 4.64-4.55 (m, 1H), 4.52-4.08 (m, 5H), 4.06-3.85 (m, 3H), 3.82-3.68 (m, 1H), 3.66-3.39 (m, 4H), 3.21-3.09 (m, 2H), 3.06-2.91 (m, 2H), 2.83-2.63 (m, 4H), 2.46-2.41 (m, 1H), 2.35-2.30 (m, 1H), 2.23-2.11 (m, 1H), 2.03-1.84 (m, 2H), 1.80-1.71 (m, 1H), 1.69-1.56 (m, 2H), 1.53 (d, J=6.0 Hz, 2H), 1.49-1.44 (m, 2H), 1.43-1.32 (m, 5H), 1.21-1.08 (m, 2H), 0.98-0.87 (m, 2H), 0.86-0.77 (m, 3H), 0.76-0.51 (m, 8H), 0.38 (s, 1H).

### Example A383. Synthesis of (3aS,6S,9S,15S,32aS)-6-isopropyl-21-(2-((S)-1-methoxyethyl)-5-(4-(oxetan-3-yl)piperazin-1-yl)pyridin-3-yl)-5,19,19-trimethyl-1-(1-methylcyclopropane-1-carbonyl)-22-(2,2,2-trifluoroethyl)-3,3a,5,6,8,9,14,15,18,19,20,22,32,32a-tetradecahydro-1H,12H-11,15-epimino-23,25-etheno-9,28-methano-26,30-(metheno)dipyrrolo[2,3-c:3′,4′-v][1,18]dioxa[6,9,12]triazacyclotriacontine-4,7,10,16(2H,13H)-tetraone

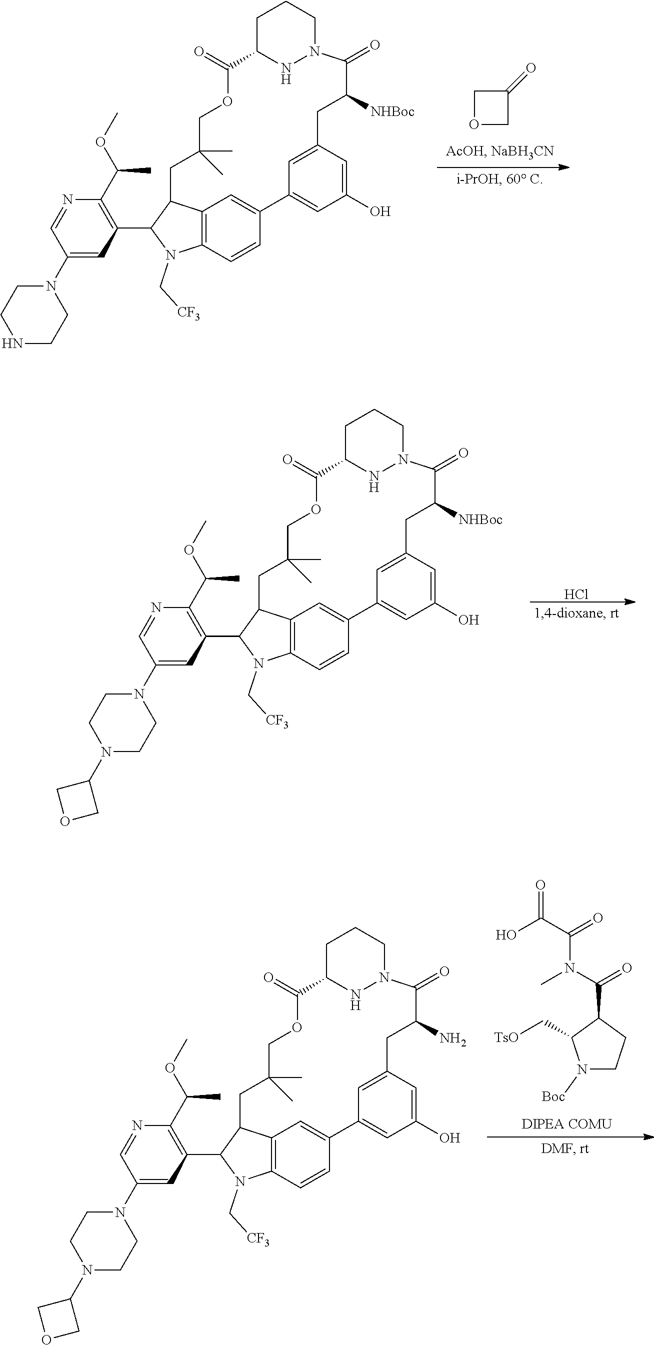

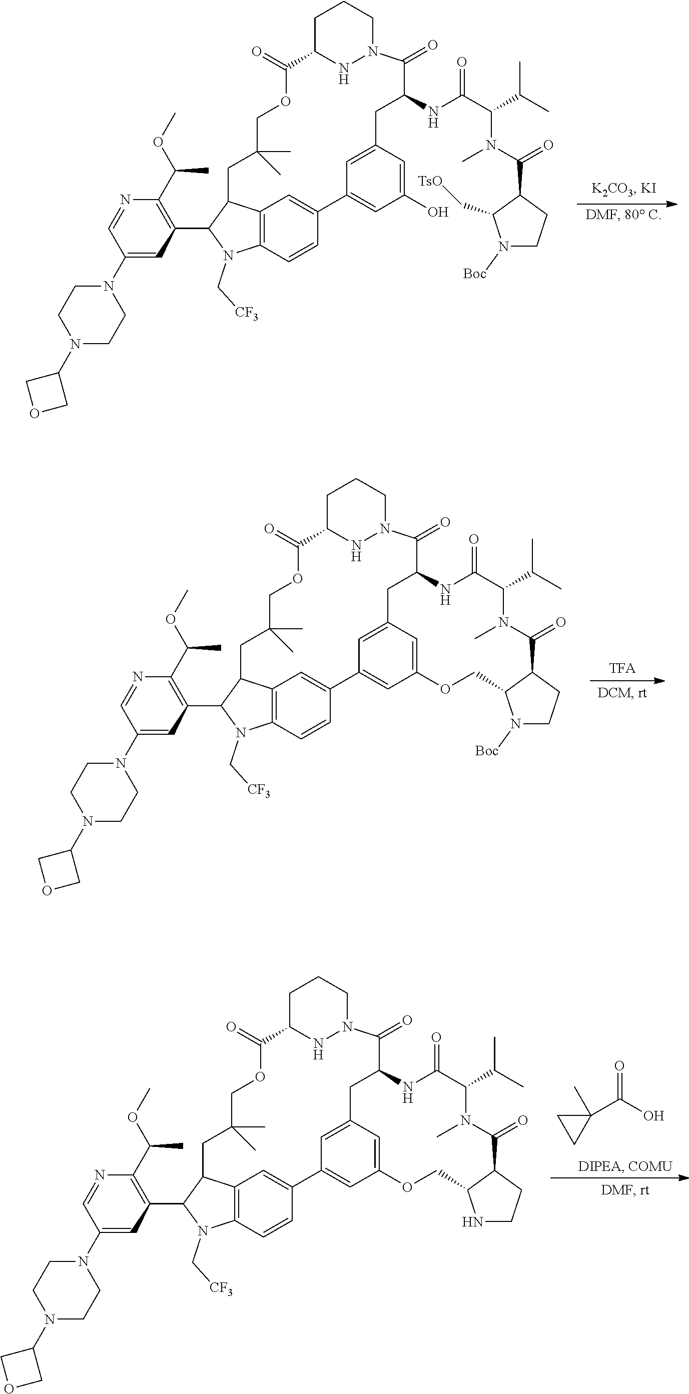

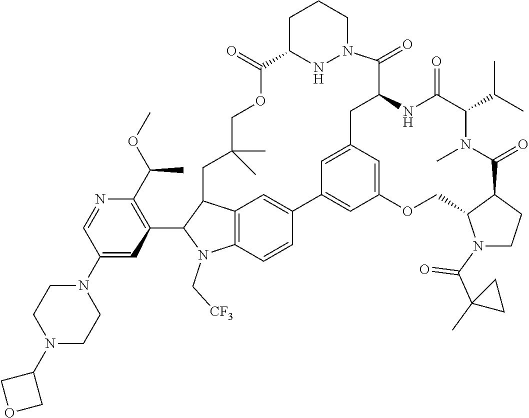

Step 1. To a mixture of tert-butyl ((6³S,4S)-2⁵-hydroxy-1²-(2-((S)-1-methoxyethyl)-5-(piperazin-1-yl)pyridin-3-yl)-10,10-dimethyl-5,7-dioxo-1¹-(2,2,2-trifluoroethyl)-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)carbamate (3.70 g, 4.21 mmol), 3-oxetanone (911 mg, 12.6 mmol) and AcOH (759 mg, 12.6 mmol) in i-PrOH (40 mL) stirred at 0° C. was added NaBH₃CN (794 mg, 12.6 mmol) in portions. The reaction mixture was stirred for 4 h at 60° C. and was then quenched with sat. aq. NaHCO₃ at 0° C. The resulting mixture was extracted with EtOAc (3×20 mL), dried over anhydrous Na₂SO₄, filtered, and concentrated under reduced pressure. The residue was purified by silica gel column chromatography to give tert-butyl ((6³S,4S)-2⁵-hydroxy-1²-(2-((S)-1-methoxyethyl)-5-(4-(oxetan-3-yl)piperazin-1-yl)pyridin-3-yl)-10,10-dimethyl-5,7-dioxo-11-(2,2,2-trifluoroethyl)-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)carbamate (2.35 g, 62% yield) as a light yellow solid. LCMS (ESI) m/z: [M+H] calcd for C₄₉H₆₂F₃N₇O₈: 934.5; found 934.4.

Step 2. To a stirred solution of tert-butyl ((6³S,4S)-2⁵-hydroxy-1²-(2-((S)-1-methoxyethyl)-5-(4-(oxetan-3-yl)piperazin-1-yl)pyridin-3-yl)-10,10-dimethyl-5,7-dioxo-1¹-(2,2,2-trifluoroethyl)-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)carbamate (2.35 g, 2.52 mmol) in 1,4-dioxane (20 mL) was added a solution of HCl in 1,4-dioxane (12 mL, 395 mmol) in portions at 0° C. The reaction mixture was stirred for 2 h at room temperature under an atmosphere of N₂. The mixture was then concentrated under reduced pressure to give (6³S,⁴S)-4-amino-2⁵-hydroxy-1²-(2-((S)-1-methoxyethyl)-5-(4-(oxetan-3-yl)piperazin-1-yl)pyridin-3-yl)-10,10-dimethyl-1¹-(2,2,2-trifluoroethyl)-6¹,6²,6³,6⁴,6⁵,6⁶-hexahydro-1¹H-8-oxa-1(5,3)-indola-6(1,3)-pyridazina-2(1,3)-benzenacycloundecaphane- 5,7-dione (1.90 g, crude) as a yellow solid. The crude product was used in the next step directly without further purification. LCMS (ESI) m/z: [M+H] calcd for C₄₄H₅₄F₃N₇₀₆: 834.4; found 834.4.

Step 3. To a solution of (6³S,⁴S)-4-amino-2⁵-hydroxy-1²-(2-((S)-1-methoxyethyl)-5-(4-(oxetan-3-yl)piperazin-1-yl)pyridin-3-yl)-10,10-dimethyl-1¹-(2,2,2-trifluoroethyl)-6¹,6²,6³,6⁴,6⁵,6⁶-hexahydro-1¹H-8-oxa-1(5,3)-indola-6(1,3)-pyridazina-2(1,3)-benzenacycloundecaphane-5,7-dione (1.90 g, crude) and DIPEA (1.47 g, 11.4 mmol) in DMF (20 mL) stirred at 0° C. under an atmosphere of N₂ were added (2S)-2-{1-[(2S,3S)-1-(tert-butoxycarbonyl)-2-{[(4-methylbenzenesulfonyl)oxy]methyl}pyrrolidin-3-yl]-N-methylformamido}-3-methylbutanoic acid (1.40 g, 2.73 mmol) and COMU (1.46 g, 3.42 mmol). The reaction mixture was stirred for 2 h at room temperature under an atmosphere of N₂. The mixture was then diluted in EtOAc (100 mL), washed with sat. aq. NH₄Cl (2×30 mL), treated with brine (2×30 mL), dried over anhydrous Na₂SO₄, filtered, and concentrated under reduced pressure. The residue was purified by silica gel column chromatography to give tert-butyl (2S,3S)-3-(((2S)-1-(((6³S,4S)-2⁵-hydroxy-1²-(2-((S)-1-methoxyethyl)-5-(4-(oxetan-3-yl)piperazin-1-yl)pyridin-3-yl)-10,10-dimethyl-5,7-dioxo-1¹-(2,2,2-trifluoroethyl)-6¹,6²,6³,6⁴,6⁵,6⁶-hexahydro-1¹H-8-oxa-1(5,3)-indola-6(1,3)-pyridazina-2(1,3)-benzenacycloundecaphane- 4-yl)amino)-3-methyl-1-oxobutan-2-yl)(methyl)carbamoyl)-2-((tosyloxy)methyl)pyrrolidine-1-carboxylate (2.30 g, 81.0% purity, 56% yield over 2 steps) as a yellow solid. LCMS (ESI) m/z: [M+H] calcd for C₆₈H₈₈F₃N₉O₁₃S: 1328.6; found 1328.5.

Step 4. To a stirred solution of tert-butyl (2S,3S)-3-(((2S)-1-(((6³S,4S)-2⁵-hydroxy-1²-(2-((S)-1-methoxyethyl)-5-(4-(oxetan-3-yl)piperazin-1-yl)pyridin-3-yl)-10,10-dimethyl-5,7-dioxo-11-(2,2,2-trifluoroethyl)-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)amino)-3-methyl-1-oxobutan-2-yl)(methyl)carbamoyl)-2-((tosyloxy)methyl)pyrrolidine-1-carboxylate (2.30 g, 81.0% purity, 1.40 mmol) in DMF (230 mL) were added K₂CO₃ (2.39 g, 17.3 mmol) and KI (0.29 g, 1.73 mmol) in portions at room temperature under an atmosphere of N₂. The reaction mixture was stirred for 2 h at 80° C. under an atmosphere of N₂. The mixture was then treated with H₂O (200 ml), extracted with EtOAc (2×200 mL), washed with brine (3×100 mL), dried over anhydrous Na₂SO₄, filtered, and concentrated under reduced pressure. The residue was purified by silica gel column chromatography to give tert-butyl (3aS,6S,9S,15S,32aS)-6-isopropyl-21-(2-((S)-1-methoxyethyl)-5-(4-(oxetan-3-yl)piperazin-1-yl)pyridin-3-yl)-5,19,19-trimethyl-4,7,10,16-tetraoxo-22-(2,2,2-trifluoroethyl)-2,3,3a,4,5,6,7,8,9,10,13,14,15,16,18,19,20,22,32,32a-icosahydro-1H,12H-11,15-epimino-23,25-etheno-9,28-methano-26,30-(metheno)dipyrrolo[2,3-c:3′,4′-v][1,18]dioxa[6,9,12]triazacyclotriacontine-1-carboxylate (1.90 g, 76% yield) as a red solid. LCMS (ESI) m/z: [M+H] calcd for C₆₁H₈₀F₃N₉O₁₀: 1156.6; found 1156.6.

Step 5. To a stirred solution of tert-butyl (3aS,6S,9S,15S,32aS)-6-isopropyl-21-(2-((S)-1-methoxyethyl)-5-(4-(oxetan-3-yl)piperazin-1-yl)pyridin-3-yl)-5,19,19-trimethyl-4,7,10,16-tetraoxo-22-(2,2,2-trifluoroethyl)-2,3,3a,4,5,6,7,8,9,10,13,14,15,16,18,19,20,22,32,32a-icosahydro-1H,12H-11,15-epimino-23,25-etheno-9,28-methano-26,30-(metheno)dipyrrolo[2,3-c:3′,4′-v][1,18]dioxa[6,9,12]triazacyclotriacontine-1-carboxylate (1.00 g, 0.865 mmol) in DCM (10 mL) was added TFA (2.5 mL, 33.7 mmol) in portions at 0° C. under an atmosphere of N₂. The reaction mixture was stirred for 2 h at room temperature and was then concentrated under reduced pressure to evaporate the DCM. The resulting mixture was treated with sat. aq. NaHCO₃ (20 mL) at 0° C., extracted with EtOAc (3×20 mL), dried over anhydrous Na₂SO₄, filtered, and concentrated under reduced pressure to give (3aS,6S,9S,15S,32aS)-6-isopropyl-21-(2-((S)-1-methoxyethyl)-5-(4-(oxetan-3-yl)piperazin-1-yl)pyridin-3-yl)-5,19,19-trimethyl-22-(2,2,2-trifluoroethyl)-3,3a,5,6,8,9,14,15,18,19,20,22,32,32a-tetradecahydro-1H,12H-11,15-epimino-23,25-etheno-9,28-methano-26,30-(metheno)dipyrrolo[2,3-c:3′,4′-v][1,18]dioxa[6,9,12]triazacyclotriacontine-4,7,10,16(2H,13H)-tetraone (800 mg, crude) as a yellow solid. LCMS (ESI) m/z: [M+H] calcd for C₅₆H₇₂F₃N₉O₈: 1056.6; found 1056.9.

Step 6. To a stirred solution of (3aS,6S,9S,15S,32aS)-6-isopropyl-21-(2-((S)-1-methoxyethyl)-5-(4-(oxetan-3-yl)piperazin-1-yl)pyridin-3-yl)-5,19,19-trimethyl-22-(2,2,2-trifluoroethyl)-3,3a,5,6,8,9,14,15,18,19,20,22,32,32a-tetradecahydro-1H,12H-11,15-epimino-23,25-etheno-9,28-methano-26,30-(metheno)dipyrrolo[2,3-c:3′,4′-v][1,18]dioxa[6,9,12]triazacyclotriacontine-4,7,10,16(2H,13H)-tetraone (100 mg, crude) and DIPEA (245 mg, 1.90 mmol) in DMF (3 mL) were added 1-methylcyclopropane-1-carboxylic acid (19.0 mg, 0.190 mmol) and COMU (81.1 mg, 0.190 mmol) dropwise at 0° C. under an atmosphere of N₂. The reaction mixture was stirred for 2 h at room temperature under an atmosphere of N₂. The resulting mixture was treated with H₂O (10 mL), extracted with EtOAc (2×10 mL), washed with brine (3×10 mL), dried over anhydrous Na₂SO₄, filtered, and concentrated under reduced pressure. The residue was purified by reversed phase chromatography to give (3aS,6S,9S,15S,32aS)-6-isopropyl-21-(2-((S)-1-methoxyethyl)-5-(4-(oxetan-3-yl)piperazin-1-yl)pyridin-3-yl)-5,19,19-trimethyl-1-(1-methylcyclopropane-1-carbonyl)-22-(2,2,2-trifluoroethyl)-3,3a,5,6,8,9,14,15,18,19,20,22,32,32a-tetradecahydro-1H,12H-11,15-epimino-23,25-etheno-9,28-methano-26,30-(metheno)dipyrrolo[2,3-c:3′,4′-v][1,18]dioxa[6,9,12]triazacyclotriacontine-4,7,10,16(2H,13H)-tetraone (14.9 mg, 12% yield over 2 steps) as a white solid. LCMS (ESI) m/z: [M+H] calcd for C₆₁H₇₈F₃N₉O₉: 1138.6; found 1138.6. ¹H NMR (400 MHz, DMSO-d₆) δ 8.48 (d, J=2.9 Hz, 1H), 8.22 (d, J=8.1 Hz, 1H), 8.01 (s, 1H), 7.84-7.66 (m, 2H), 7.36 (s, 1H), 7.27 (s, 1H), 7.21 (d, J=2.8 Hz, 1H), 6.77 (s, 1H), 5.53-5.42 (m, 1H), 5.30 (s, 1H), 5.20 (d, J=12.2 Hz, 1H), 4.85 (dd, J=16.6, 9.1 Hz, 1H), 4.74 (d, J=11.1 Hz, 1H), 4.56 (t, J=6.5 Hz, 4H), 4.46 (t, J=6.1 Hz, 3H), 4.42-4.13 (m, 5H), 4.06 (d, J=6.3 Hz, 1H), 3.71 (d, J=11.3 Hz, 1H), 3.64 (d, J=11.1 Hz, 1H), 3.56 (s, 1H), 3.25 (s, 1H), 3.17 (s, 2H), 2.91 (d, J=14.8 Hz, 1H), 2.84 (s, 3H), 2.68 (s, 1H), 2.55 (d, J=4.1 Hz, 3H), 2.42 (d, J=5.2 Hz, 5H), 2.25 (s, 2H), 2.02 (dd, J=11.2, 7.1 Hz, 2H), 1.82 (s, 1H), 1.70 (dd, J=11.6, 7.8 Hz, 1H), 1.38-1.28 (m, 7H), 1.22 (d, J=11.1 Hz, 2H), 1.04 (dd, J=6.1, 3.6 Hz, 1H), 0.97 (s, 1H), 0.87 (s, 3H), 0.84-0.67 (m, 6H), 0.66-0.57 (m, 2H), 0.48 (p, J=4.4 Hz, 1H), 0.41 (s, 3H).

### Example A384. Synthesis of (3aS,6S,9S,15S,32aS)-21-(1-(1-cyclopropylpiperidin-4-yl)-3-((S)-1-methoxyethyl)-1H-pyrazol-4-yl)-22-ethyl-1-(2-fluoro-2-methylpropanoyl)-6-isopropyl-5,19,19-trimethyl-3,3a,5,6,8,9,14,15,18,19,20,22,32,32a-tetradecahydro-1H,12H-11,15-epimino-23,25-etheno-9,28-methano-26,30-(metheno)dipyrrolo[2,3-c:3′,4′-v][1,18]dioxa[6,9,12]triazacyclotriacontine-4,7,10,16(2H,13H)-tetraone

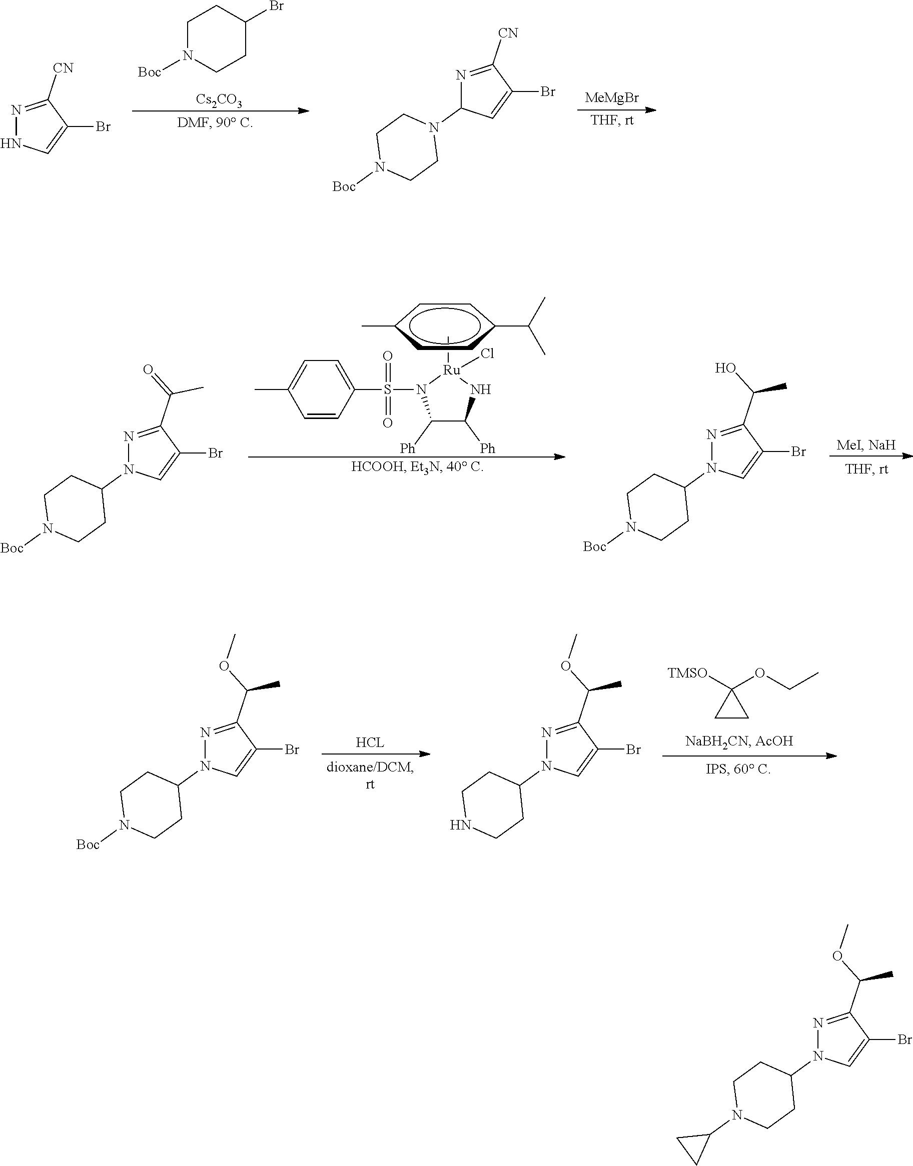

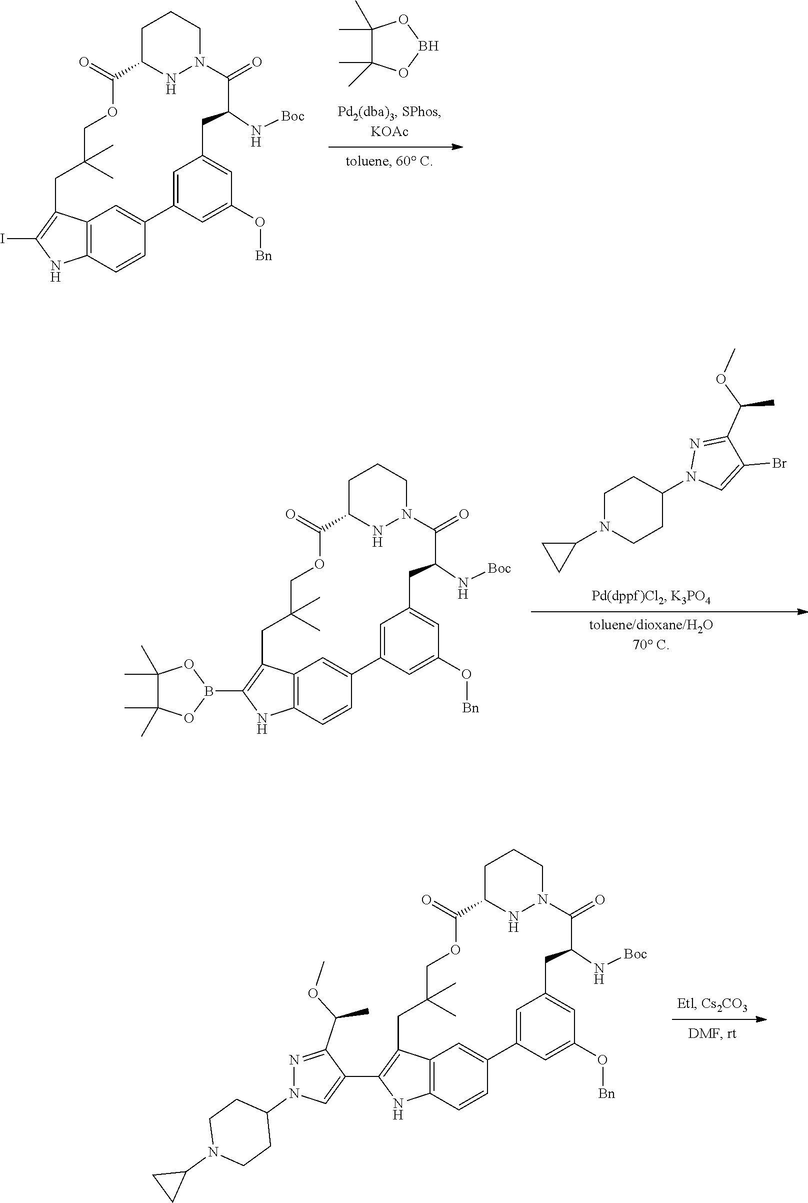

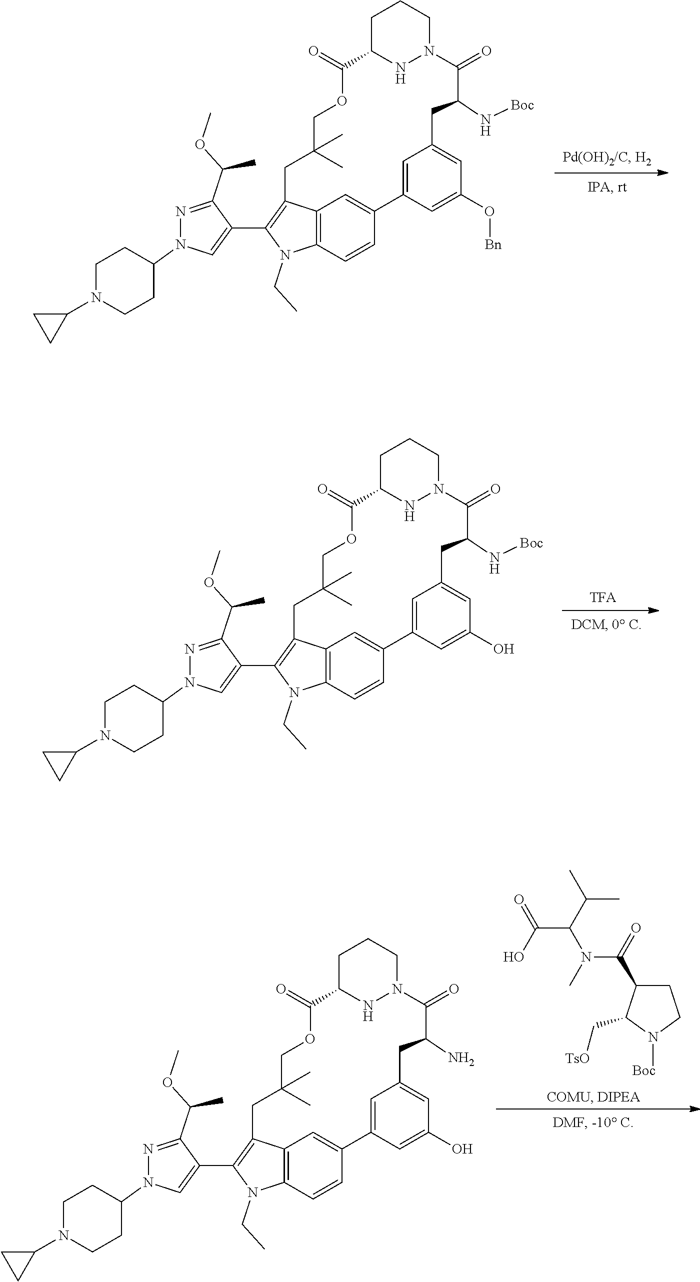 [SMILES: CCn1c(-c2cn(C3CCN(C4CC4)CC3)nc2[C@H](C)OC)c2c3cc(ccc31)-c1cc(cc(OCc3ccccc3)c1)C[C@H](NC(=O)OC(C)(C)C)C(=O)N1CCC[C@H](N1)C(=O)OCC(C)(C)C2 ; CCn1c(-c2cn(C3CCN(C4CC4)CC3)nc2[C@H](C)OC)c2c3cc(ccc31)-c1cc(O)cc(c1)C[C@H](NC(=O)OC(C)(C)C)C(=O)N1CCC[C@H](N1)C(=O)OCC(C)(C)C2 ; CCn1c(-c2cn(C3CCN(C4CC4)CC3)nc2[C@H](C)OC)c2c3cc(ccc31)-c1cc(O)cc(c1)C[C@H](N)C(=O)N1CCC[C@H](N1)C(=O)OCC(C)(C)C2 ; Cc1ccc(S(=O)(=O)OC[C@@H]2[C@@H](C(=O)N(C)C(C(=O)O)C(C)C)CCN2C(=O)OC(C)(C)C)cc1]

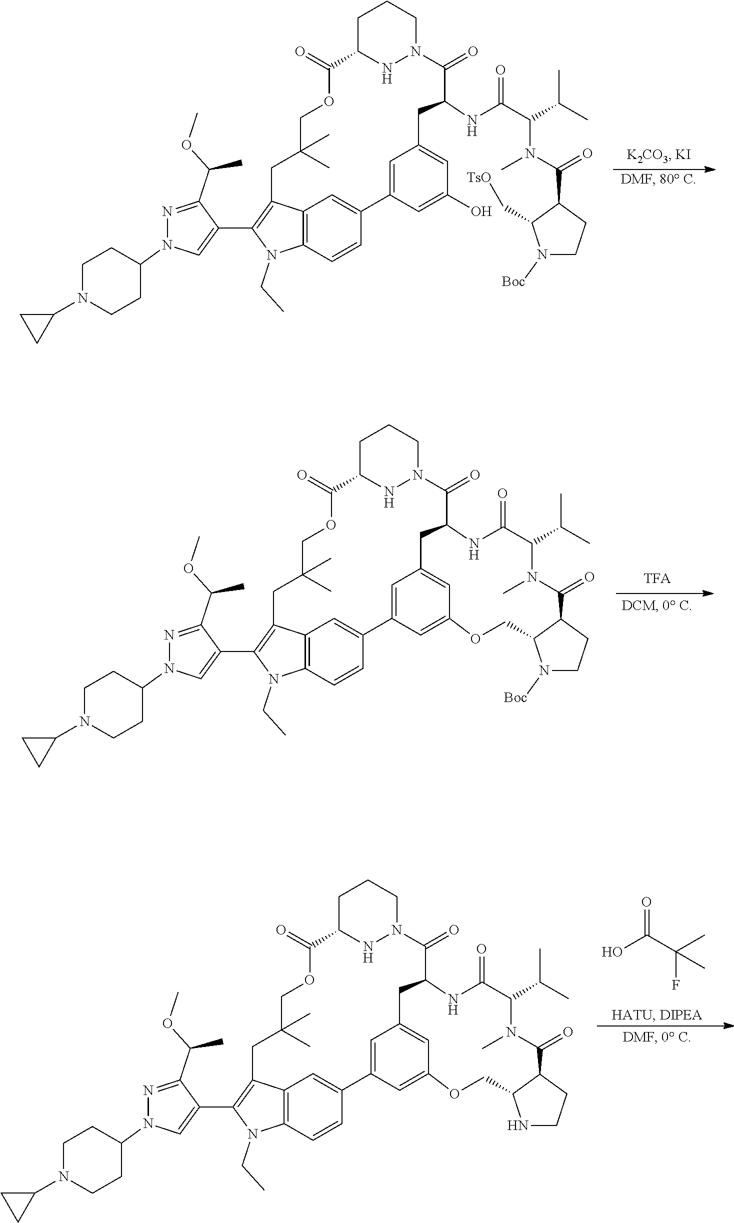

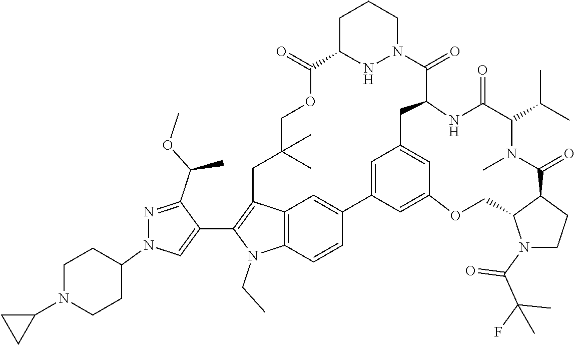

Step 1. To a solution of 4-bromo-1H-pyrazole-3-carbonitrile (25 g, 145 mmol) and tert-butyl 4-bromopiperidine-1-carboxylate (76.8 g, 290 mmol) in DMF (200 mL) at room temperature was added Cs₂CO₃ (118 g, 363 mmol). The resulting mixture was stirred for 16 h at 90° C. and was then quenched at 0° C. by the addition of H₂O (600 mL). The aqueous mixture was extracted with EtOAc (3×300 mL), and the combined organic extracts were washed with brine (2×200 mL), dried over Na₂SO₄, filtered, and concentrated under reduced pressure. Purification by normal phase chromatography yielded tert-butyl 4-(4-bromo-3-cyano-1H-pyrazol-1-yl)piperidine-1-carboxylate (35 g, 68% yield) as a yellow solid. LCMS (ESI) m/z: [M+H−C₄H₁₀] calcd for C₁₄H₁₉BrN₄O₃: 340.0; found 340.0.

Step 2. To a solution of MeMgBr (113 g, 943 mmol) in THE (335 mL) stirred at −10° C. was added tert-butyl 4-(4-bromo-3-cyano-1H-pyrazol-1-yl)piperidine-1-carboxylate (33.5 g, 94.3 mmol). The resulting mixture was stirred for 16 h at room temperature and was then acidified to pH=5 by the addition of conc. HCl. The aqueous mixture was extracted with EtOAc (3×400 mL), and the combined organic extracts were washed with brine (2×300 mL), dried over Na₂SO₄, filtered, and concentrated under reduced pressure. Purification by normal phase chromatography afforded tert-butyl 4-(3-acetyl-4-bromo-1H-pyrazol-1-yl)piperidine-1-carboxylate (18 g, 51% yield) as a yellow solid. LCMS (ESI) m/z: [M+H—C₄H₁₀] calcd for C₁₄H₁₉BrN₄O₃: 316.0; found 316.0.

Step 3. To a solution of formic acid (6.8 g, 48 mmol) and Et₃N (75 g, 741 mmol) stirred at 0° C. were added RuCl(p-cymene)[(S,S)-Ts-DPEN](200 mg, 0.31 mmol) and tert-butyl 4-(3-acetyl-4-bromo-1H-pyrazol-1-yl)piperidine-1-carboxylate (23 g, 62 mmol). The resulting mixture was stirred for 2 h at 40° C. and quenched at 0° C. by the addition of H₂O (500 mL). The aqueous mixture was extracted with EtOAc (3×500 mL), and the combined organic extracts were washed with brine (3×800 mL), dried over Na₂SO₄, filtered, and concentrated under reduced pressure. Purification by normal phase chromatography afforded tert-butyl (S)-4-(4-bromo-3-(1-hydroxyethyl)-1H-pyrazol-1-yl)piperidine-1-carboxylate (20 g, 86% yield) as a yellow oil. LCMS (ESI) m/z: [M+H] calcd for C₂₄H₁₅BrN₃O₃: 374.1; found 371.1.

Step 4. To a solution of tert-butyl (S)-4-(4-bromo-3-(1-hydroxyethyl)-1H-pyrazol-1-yl)piperidine-1-carboxylate (19 g, 50 mmol) in THE (190 mL) at 0° C. was added NaH (2.44 g, 101 mmol). The resulting mixture was stirred for 1 h at room temperature, after which time Mel (14.4 g, 101 mmol) was added at 0° C. The reaction mixture was stirred for 2 h at room temperature and was then quenched at 0° C. by the addition of sat. aq. NH₄Cl. The aqueous mixture was extracted with EtOAc (3×300 mL), and the combined organic extracts were dried over Na₂SO₄, filtered, and concentrated under reduced pressure to afford tert-butyl (S)-4-(4-bromo-3-(1-methoxyethyl)-1H-pyrazol-1-yl)piperidine-1-carboxylate (19 g, crude), which was used in the next reaction without further purification. LCMS (ESI) m/z: [M+H] calcd for C₁₆H₂₆BrN₃O₃: 388.1; found 388.0.

Step 5. To a solution of tert-butyl (S)-4-(4-bromo-3-(1-methoxyethyl)-1H-pyrazol-1-yl)piperidine-1-carboxylate (1.0 g, crude) in DCM (10 mL) at 0° C. was added 4 M HCl in dioxane (10 mL). The resulting mixture was stirred for 1 h at room temperature and was then concentrated under reduced pressure to afford (S)-4-(4-bromo-3-(1-methoxyethyl)-1H-pyrazol-1-yl)piperidine (1.1 g, crude), which was taken directly to the next step without further purification. LCMS (ESI) m/z: [M+H] calcd for C₁₁H₁₈BrN₃O: 288.1; found 288.0.

Step 6. To a solution of (S)-4-(4-bromo-3-(1-methoxyethyl)-1H-pyrazol-1-yl)piperidine (1.1 g, crude) and acetic acid (460 mg, 7.63 mmol) in IPA (11 mL) stirred at room temperature were added NaBH₃CN (1.20 g, 19.1 mmol) and (1-ethoxycyclopropoxy)trimethylsilane (6.65 g, 38.2 mmol). The resulting mixture was stirred for 2 h at 60° C. and was then neutralized by the addition of sat. aq. NaHCO₃. The aqueous mixture was extracted with EtOAc (3×70 mL), and the combined organic extracts were dried over Na₂SO₄, filtered, and concentrated under reduced pressure. Purification by reversed phase chromatography afforded (S)-4-(4-bromo-3-(1-methoxyethyl)-1H-pyrazol-1-yl)-1-cyclopropylpiperidine (720 mg, 83% yield over 3 steps) as a white solid. LCMS (ESI) m/z: [M+H] calcd for C₁₄H₂₂BrN₃O: 328.1; found 328.0.

Step 7. To a solution of tert-butyl ((6³S,4S)-2⁵-(benzyloxy)-1²-iodo-10,10-dimethyl-5,7-dioxo-6¹,6²,6³,6⁴,6⁵,6⁶-hexahydro-11H-8-oxa-1(5,3)-indola-6(1,3)-pyridazina-2(1,3)-benzenacycloundecaphane-4-yl)carbamate (2.0 g, 2.5 mmol) and KOAc (870 mg, 8.83 mmol) in toluene (20 mL) stirred at room temperature were added SPhos (310 mg, 757 μmol), 4,4,5,5-tetramethyl-1,3,2-dioxaborolane (2.42 g, 18.9 mmol), and Pd₂(dba)₃ (280 mg, 303 μmol). The resulting mixture was stirred for 3 h at 60° C. and was then quenched at 0° C. by the addition of sat. aq. NH₄Cl (100 mL). The aqueous mixture was extracted with EtOAc (3×100 mL), and the combined organic extracts were washed with brine (1×200 mL), dried over Na₂SO₄, filtered, and concentrated under reduced pressure. Purification by normal phase chromatography afforded tert-butyl ((6³S,4S)-2⁵-(benzyloxy)-10,10-dimethyl-5,7-dioxo-1²-(4,4,5,5-tetramethyl-1,3,2-dioxaborolan-2-yl)-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)carbamate (1.3 g, 65% yield) as a yellow solid. LCMS (ESI) m/z: [M+Na] calcd for C₄₅H₅₇BN₄O₈: 815.4; found 815.2.

Step 8. To a solution of tert-butyl ((6³S,4S)-2⁵-(benzyloxy)-10,10-dimethyl-5,7-dioxo-1²-(4,4,5,5-tetramethyl-1,3,2-dioxaborolan-2-yl)-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)carbamate (1.22 g, 1.54 mmol) and K₃PO₄ (820 mg, 3.85 mmol) in dioxane (4.0 mL) and H₂O (2.0 mL) stirred at room temperature were added (S)-4-(4-bromo-3-(1-methoxyethyl)-1H-pyrazol-1-yl)-1-cyclopropylpiperidine (610 mg, 1.85 mmol) and Pd(dppfCl₂ (110 mg, 154 mmol). The resulting mixture was stirred overnight at 70° C. and was then quenched at 0° C. by the addition of H₂O (100 mL). The aqueous mixture was extracted with EtOAc (3×100 mL), and the combined organic extracts were dried over Na₂SO₄, filtered, and concentrated under reduced pressure. Purification by normal phase chromatography afforded tert-butyl ((6³S,4S)-2⁵-(benzyloxy)-1²-(1-(1-cyclopropylpiperidin-4-yl)-3-((S)-1-methoxyethyl)-1H-pyrazol-4-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)carbamate (800 mg, 57% yield) as a brown solid. LCMS (ESI) m/z: [M+H] calcd for C₅₃H₆₇N₇O₇: 914.5; found 914.4.

Step 9. To a solution of tert-butyl ((6³S,4S)-2⁵-(benzyloxy)-1²-(1-(1-cyclopropylpiperidin-4-yl)-3-((S)-1-methoxyethyl)-1H-pyrazol-4-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)carbamate (800 mg, 0.875 mmol) in DMF (8.0 mL) at 0° C. were added Cs₂CO₃ (1.43 g, 4.38 mmol) and iodoethane (682 mg, 4.38 mmol). The resulting mixture was stirred overnight at room temperature and was then quenched at 0° C. by the addition of cold H₂O (200 mL). The aqueous mixture was extracted with EtOAc (3×100 mL), and the combined organic extracts were dried over Na₂SO₄, filtered, and concentrated under reduced pressure. Purification by normal phase chromatography afforded tert-butyl ((6³S,4S)-2⁵-(benzyloxy)-1²-(1-(1-cyclopropylpiperidin-4-yl)-3-((S)-1-methoxyethyl)-1H-pyrazol-4-yl)-1¹-ethyl-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)carbamate (650 mg, 79% yield) as a yellow solid. LCMS (ESI) m/z: [M+H] calcd for C₅₅H₇₁N₇O₇: 942.6; found 942.6.

Step 10. To a stirred solution of tert-butyl ((6³S,4S)-2⁵-(benzyloxy)-1²-(1-(1-cyclopropylpiperidin-4-yl)-3-((S)-1-methoxyethyl)-1H-pyrazol-4-yl)-1¹-ethyl-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)carbamate (560 mg, 0.594 mmol) in IPA (10 mL) were added Pd/C (300 mg) and Pd(OH)₂/C (300 mg). The resulting mixture was stirred overnight at room temperature under an atmosphere of H₂, filtered, the filter cake washed with 1:1 vol. DCM/MeOH (3×150 mL), and the filtrate concentrated under reduced pressure. Purification of the resulting filtrate material by normal phase chromatography afforded tert-butyl ((6³S,4S)-1²-(1-(1-cyclopropylpiperidin-4-yl)-3-((S)-1-methoxyethyl)-1H-pyrazol-4-yl)-1¹-ethyl-2⁵-hydroxy-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)carbamate (390 mg, 77% yield) as a yellow solid. LCMS (ESI) m/z: [M+H] calcd for C₄₈H₆₅N₇O₇: 852.5; found 852.4.

Step 11. To a solution of tert-butyl ((6³S,4S)-1²-(1-(1-cyclopropylpiperidin-4-yl)-3-((S)-1-methoxyethyl)-1H-pyrazol-4-yl)-1¹-ethyl-2⁵-hydroxy-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)carbamate (390 mg, 0.458 mmol) in DCM (4.0 mL) stirred at 0° C. was added TFA (2.0 mL). The resulting mixture was stirred for 1 h at 0° C. and was then neutralized by the addition of sat. aq. NaHCO₃. The aqueous phase was extracted with EtOAc (3×100 mL), and the combined organic extracts were dried over Na₂SO₄, filtered, and concentrated under reduced pressure to afford (6³S,4S)-4-amino-1²-(1-(1-cyclopropylpiperidin-4-yl)-3-((S)-1-methoxyethyl)-1H-pyrazol-4-yl)-1¹-ethyl-2⁵-hydroxy-10,10-dimethyl-6¹,6²,6³,6⁴,6⁵,6⁶-hexahydro-1¹H-8-oxa-1(5,3)-indola-6(1,3)-pyridazina-2(1,3)-benzenacycloundecaphane-5,7-dione (400 mg, crude) which was taken directly to the next reaction without further purification. LCMS (ESI) m/z: [M/2+H] calcd for C₄₃H₅₇N₇O₅: 376.7; found 376.8.

Step 12. To a stirred solution of (6³S,4S)-4-amino-1²-(1-(1-cyclopropylpiperidin-4-yl)-3-((S)-1-methoxyethyl)-1H-pyrazol-4-yl)-1¹-ethyl-2⁵-hydroxy-10,10-dimethyl-6¹,6²,6³,6⁴,6⁵,6⁶-hexahydro-1¹H-8-oxa-1(5,3)-indola-6(1,3)-pyridazina-2(1,3)-benzenacycloundecaphane-5,7-dione (400 mg, crude) and DIPEA (2.75 g, 21.3 mmol) in DMF (4.0 mL) at −10° C. were added (2S)-2-{1-[(2S,3S)-1-(tert-butoxycarbonyl)-2-{[(4-methylbenzenesulfonyl)oxy]methyl}pyrrolidin-3-yl]-N-methylformamido}-3-methylbutanoic acid (409 mg, 0.798 mmol) and COMU (318 mg, 0.745 mmol). The resulting mixture was stirred for 1 h at −10° C. and was then quenched at 0° C. by the addition of H₂O (50 mL). The aqueous mixture was extracted with EtOAc (3×100 mL), and the combined organic extracts were dried over Na₂SO₄, filtered, and concentrated under reduced pressure. Purification by normal phase chromatography afforded tert-butyl (2S,3S)-3-(((2S)-1-(((6³S,4S)-1²-(1-(1-cyclopropylpiperidin-4-yl)-3-((S)-1-methoxyethyl)-1H-pyrazol-4-yl)-1¹-ethyl-2⁵-hydroxy-10,10-dimethyl-5,7-dioxo-6¹,6²,6³,6⁴,6⁵,6⁶-hexahydro-1¹H-8-oxa-1(5,3)-indola-6(1,3)-pyridazina- 2(1,3)-benzenacycloundecaphane-4-yl)amino)-3-methyl-1-oxobutan-2-yl)(methyl)carbamoyl)-2-((tosyloxy)methyl)pyrrolidine-1-carboxylate (450 mg, 79% yield over 2 steps) as a yellow solid. LCMS (ESI) m/z: [M+H] calcd for C₆₇H₉₁N₉O₁₂S: 1246.7; found 1247.3.

Step 13. To a solution of tert-butyl (2S,3S)-3-(((2S)-1-(((6³S,4S)-1²-(1-(1-cyclopropylpiperidin-4-yl)-3-((S)-1-methoxyethyl)-1H-pyrazol-4-yl)-1¹-ethyl-2⁵-hydroxy-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)amino)-3-methyl-1-oxobutan-2-yl)(methyl)carbamoyl)-2-((tosyloxy)methyl)pyrrolidine-1-carboxylate (430 mg, 0.345 mmol) in DMF (43 mL) stirred at room temperature were added K₂CO₃ (476 mg, 3.45 mmol) and KI (57.3 mg, 0.345 mmol). The resulting mixture was stirred for 2 h at 80° C. and was then diluted by the addition of H₂O (200 mL). The aqueous mixture was extracted with EtOAc (3×100 mL), and the combined organic extracts were washed with H₂O (3×150 mL), dried over Na₂SO₄, filtered, and concentrated under reduced pressure. Purification by reversed phase chromatography afforded tert-butyl (3aS,6S,9S,15S,32aS)-21-(1-(1-cyclopropylpiperidin-4-yl)-3-((S)-1-methoxyethyl)-1H-pyrazol-4-yl)-22-ethyl-6-isopropyl-5,19,19-trimethyl-4,7,10,16-tetraoxo-2,3,3a,4,5,6,7,8,9,10,13,14,15,16,18,19,20,22,32,32a-icosahydro-1H,12H-11,15-epimino-23,25-etheno-9,28-methano-26,30-(metheno)dipyrrolo[2,3-c:3′,4′-v][1,18]dioxa[6,9,12]triazacyclotriacontine-1-carboxylate 200 mg, 54% yield) as a yellow solid. LCMS (ESI) m/z: [M+H] calcd for C₆₀H₈₃N₉O₉: 1074.6; found 1074.6.

Step 14. To a solution of tert-butyl (3aS,6S,9S,15S,32aS)-21-(1-(1-cyclopropylpiperidin-4-yl)-3-((S)-1-methoxyethyl)-1H-pyrazol-4-yl)-22-ethyl-6-isopropyl-5,19,19-trimethyl-4,7,10,16-tetraoxo-2,3,3a,4,5,6,7,8,9,10,13,14,15,16,18,19,20,22,32,32a-icosahydro-1H,12H-11,15-epimino-23,25-etheno-9,28-methano-26,30-(metheno)dipyrrolo[2,3-c:3′,4′-v][1,18]dioxa[6,9,12]triazacyclotriacontine-1-carboxylate (195 mg, 0.182 mmol) in DCM (2.0 mL) stirred at 0° C. was added TFA (1.0 mL). The resulting mixture was stirred for 1 h at 0° C. and was then basified to pH=8 by the addition of sat. aq. NaHCO₃. The aqueous phase was extracted with EtOAc (3×50 mL), and the combined organic extracts were dried over Na₂SO₄, filtered, and concentrated under reduced pressure to afford (3aS,6S,9S,15S,32aS)-21-(1-(1-cyclopropylpiperidin-4-yl)-3-((S)-1-methoxyethyl)-1H-pyrazol-4-yl)-22-ethyl-6-isopropyl-5,19,19-trimethyl-3,3a,5,6,8,9,14,15,18,19,20,22,32,32a-tetradecahydro-1H,12H-11,15-epimino-23,25-etheno-9,28-methano-26,30-(metheno)dipyrrolo[2,3-c:3′,4′-v][1,18]dioxa[6,9,12]triazacyclotriacontine-4,7,10,16(2H,13H)-tetraone (200 mg, crude) which was taken directly to the next reaction without further purification. LCMS (ESI) m/z: [M+H] calcd for C₅₅H₇₅N₉O₇: 974.6; found 974.5.

Step 15. To a solution of (3aS,6S,9S,15S,32aS)-21-(1-(1-cyclopropylpiperidin-4-yl)-3-((S)-1-methoxyethyl)-1H-pyrazol-4-yl)-22-ethyl-6-isopropyl-5,19,19-trimethyl-3,3a,5,6,8,9,14,15,18,19,20,22,32,32a-tetradecahydro-1H,12H-11,15-epimino-23,25-etheno-9,28-methano-26,30-(metheno)dipyrrolo[2,3-c:3′,4′-v][1,18]dioxa[6,9,12]triazacyclotriacontine-4,7,10,16(2H,13H)-tetraone (200 mg, crude) and DIPEA (1.06 g, 8.20 mmol) in DMF (2.0 mL) stirred at 0° C. were added 2-fluoro-2-methylpropanoic acid (43.6 mg, 0.410 mmol) and HATU (110 mg, 0.256 mmol). The resulting mixture was stirred for 1 h at 0° C. and was then quenched by the addition of H₂O (50 mL). The aqueous mixture was extracted with EtOAc (3×30 mL), and the combined organic extracts were washed with H₂O (3×30 mL), dried over Na₂SO₄, filtered, and concentrated under reduced pressure. Purification by reversed phase chromatography afforded (3aS,6S,9S,15S,32aS)-21-(1-(1-cyclopropylpiperidin-4-yl)-3-((S)-1-methoxyethyl)-1H-pyrazol-4-yl)-22-ethyl-1-(2-fluoro-2-methylpropanoyl)-6-isopropyl-5,19,19-trimethyl-3,3a,5,6,8,9,14,15,18,19,20,22,32,32a-tetradecahydro-1H,12H-11,15-epimino-23,25-etheno-9,28-methano-26,30-(metheno)dipyrrolo[2,3-c:3′,4′-v][1,18]dioxa[6,9,12]triazacyclotriacontine-4,7,10,16(2H,13H)-tetraone (108 mg, 56% yield over 2 steps) as a white solid. LCMS (ESI) m/z: [M+H] calcd for C₅₉H₈₀FN₉O₈: 1062.6; found 1062.5. ¹H-NMR (400 MHz, DMSO-d₆) δ 9.04 (br s, 1H), 8.35-8.08 (m, 1H), 8.06-7.87 (m, 1H), 7.68 (d, J=8.1 Hz, 1H), 7.56-7.28 (m, 2H), 6.76 (t, J=9.0 Hz, 1H), 5.52-5.18 (m, 1H), 5.18-4.91 (m, 1H), 4.81-4.52 (m, 2H), 4.49-4.16 (m, 3H), 4.13-3.97 (m, 2H), 3.96-3.74 (m, 2H), 3.25 (s, 3H), 3.13 (s, 3H), 3.08-2.99 (m, 1H), 2.95-2.78 (m, 3H), 2.56 (s, 1H), 2.39 (s, 2H), 2.35-2.16 (m, 4H), 2.09-1.96 (m, 3H), 1.88-1.65 (m, 4H), 1.60 (s, 3H), 1.55 (dd, J=3.8, 2.3 Hz, 4H), 1.50-1.40 (m, 2H), 1.32 (d, J=6.4 Hz, 1H), 1.20 (ft, J=14.9, 7.3 Hz, 4H), 1.10 (t, J=7.0 Hz, 2H), 1.05-0.92 (m, 5H), 0.91-0.84 (m, 4H), 0.84-0.65 (m, 9H), 0.51 (s, 3H).

### Example A398. Synthesis of (2aS,5S,8S,14S,31aS)-20-(5-(4-cyclopropylpiperazin-1-yl)-2-((S)-1-methoxyethyl)pyridin-3-yl)-21-ethyl-1-(2-fluoro-2-methylpropanoyl)-5-isopropyl-4,18,18-trimethyl-2,2a,4,5,7,8,13,14,17,18,19,21,31,31a-tetradecahydro-11H-10,14-epimino-22,24-etheno-8,27-methano-25,29-(metheno)azeto[2,3-c]pyrrolo[3,4-v][1,18]dioxa[6,9,12]triazacyclotriacontine-3,6,9,15(1H,12H)-tetraone

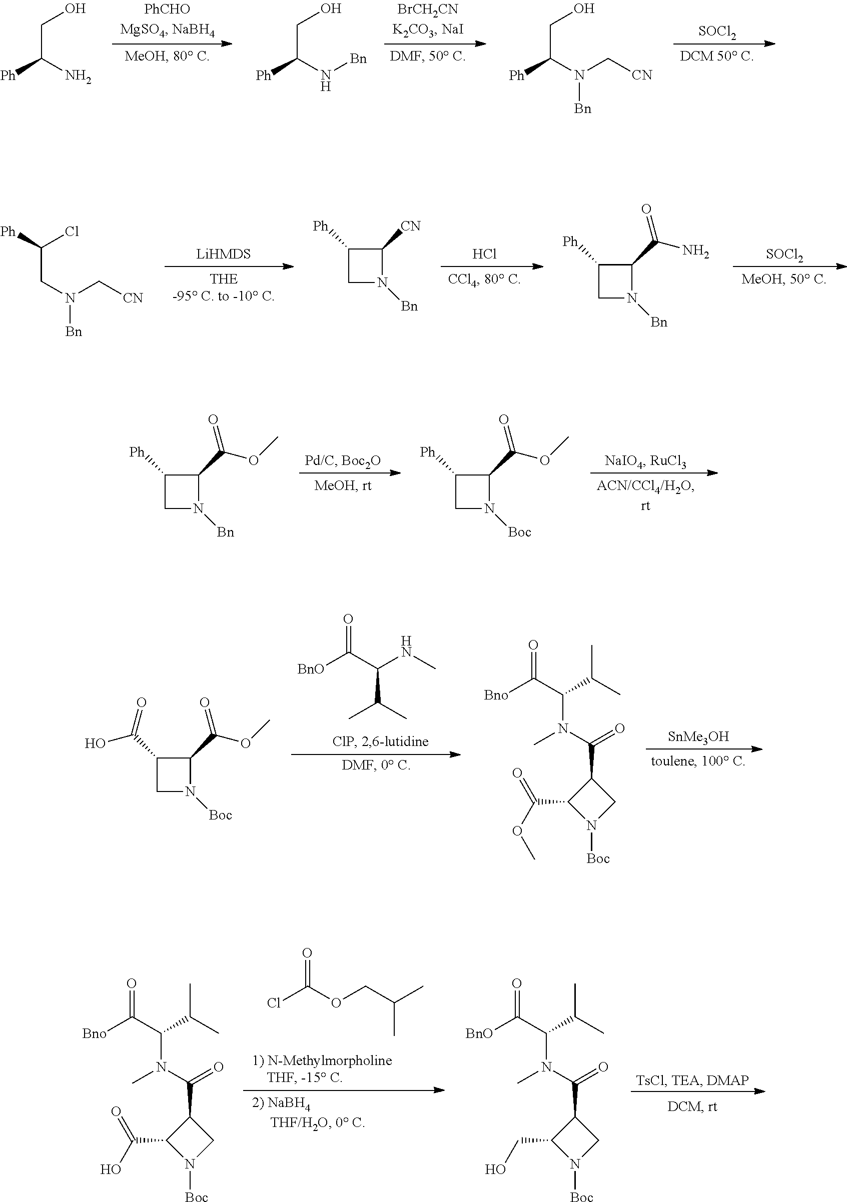

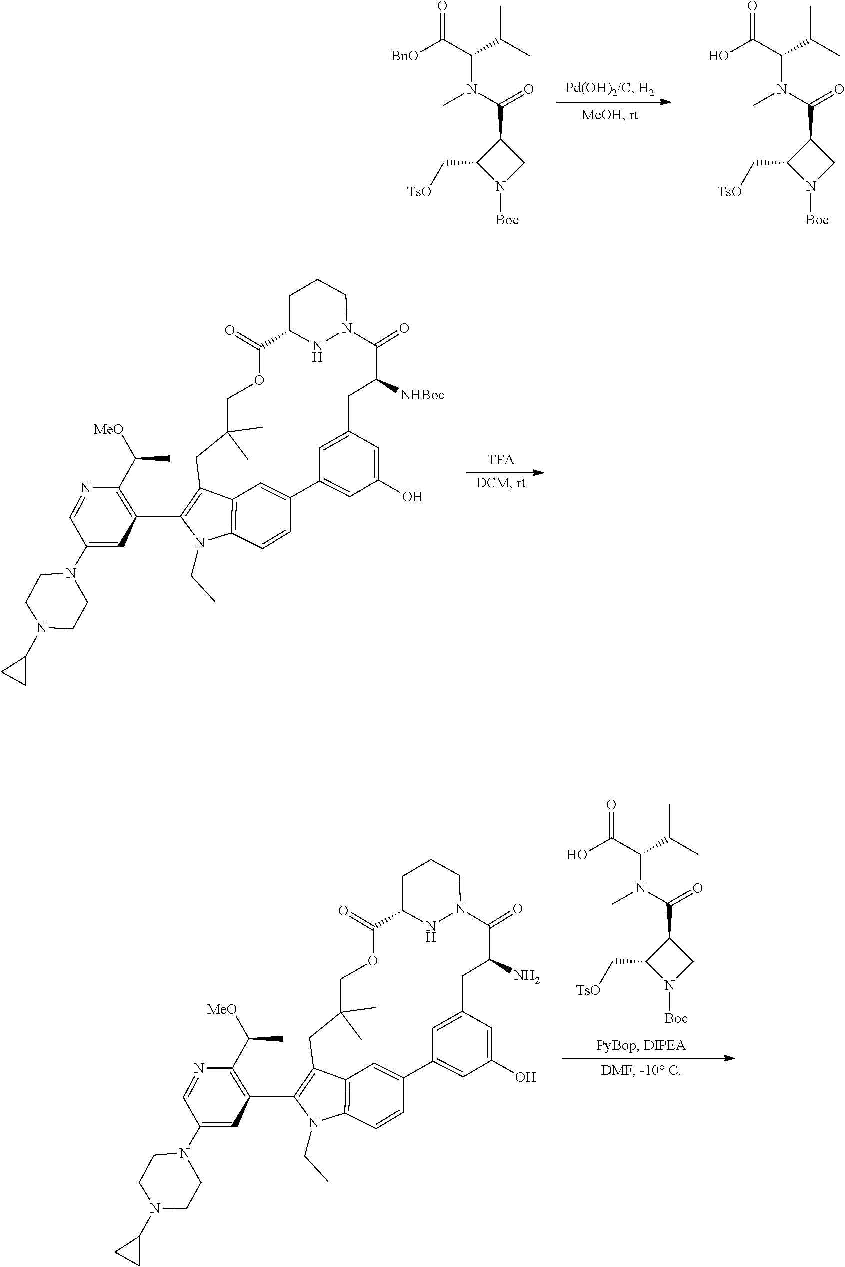

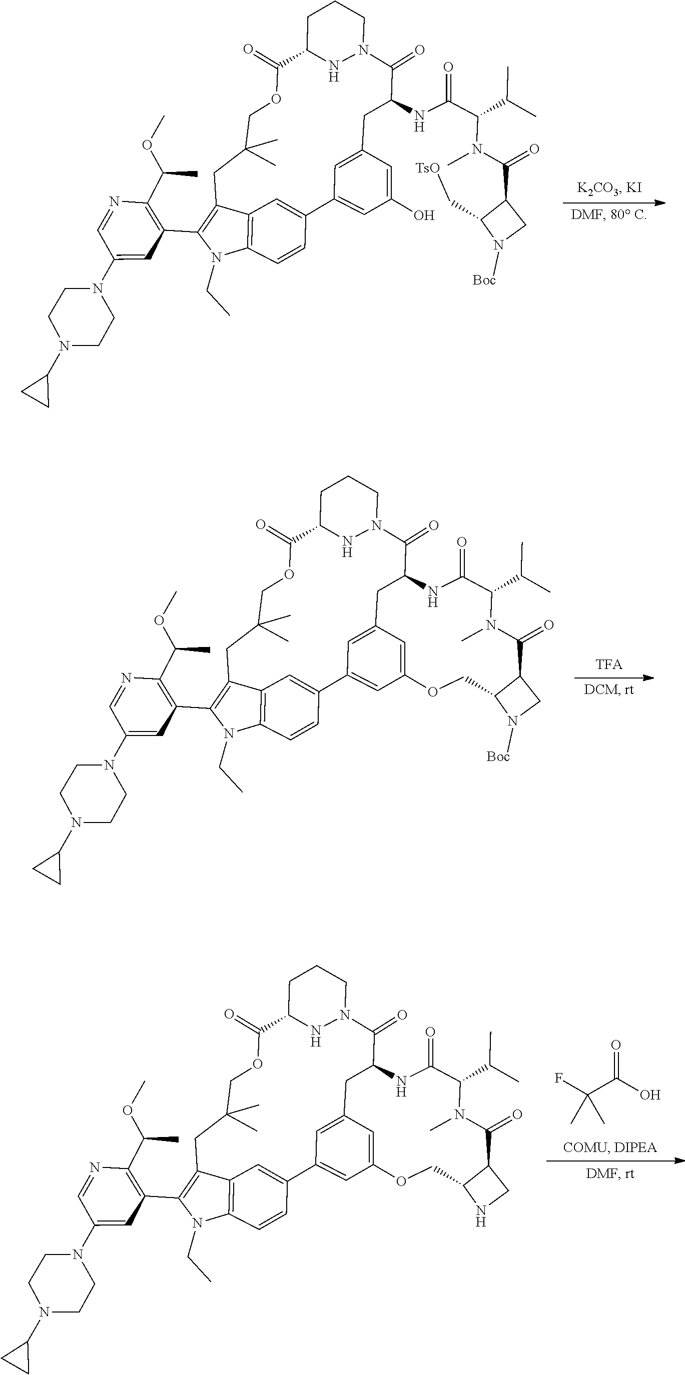

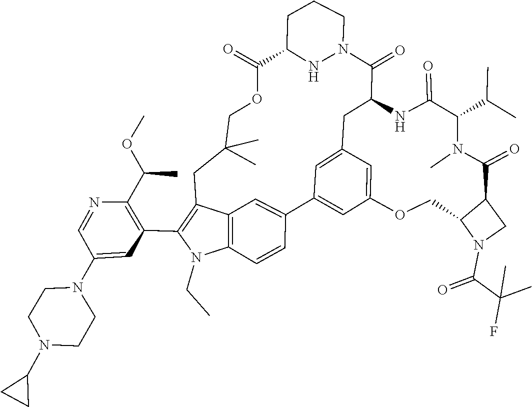

Step 1. To a stirred solution of (S)-2-amino-2-phenylethan-1-ol (10.0 g, 72.9 mmol) in MeOH (200 mL) was added MgSO₄ (26.3 g, 219 mmol) in portions at room temperature. To the mixture was added benzaldehyde (7.74 g, 72.9 mmol) in MeOH (200 mL), followed by NaBH₄ (6.89 g, 182 mmol) portionwise at room temperature. After 30 minutes, the resulting mixture was heated to 80° C. and stirred under an atmosphere of N₂. After 12 h, the reaction was quenched with H₂O at 0° C., the resulting mixture was filtered, and the filter cake was washed with MeOH (5×50 mL). The filtrate was concentrated under reduced pressure and the resulting mixture was extracted with EtOAc (3×400 mL). The combined organic extracts were dried over Na₂SO₄, filtered, concentrated under reduced pressure, and the residue was purified by silica gel column chromatography to afford (S)-2-(benzylamino)-2-phenylethan-1-ol (14.0 g, 84% yield) as a white solid. LCMS (ESI) m/z: [M+H] Calcd for C₁₅H₁₇NO: 228.1; found 228.1.

Step 2. A stirred mixture of (S)-2-(benzylamino)-2-phenylethan-1-ol (10.0 g, 44 mmol), sodium iodide (39.6 g, 264 mmol), 2-bromoacetonitrile (31.7 g, 264 mmol), and K₂CO₃ (36.5 g, 264 mmol) in DMF (100 mL) was heated to 50° C. After 12 h, the reaction was cooled to 0° C. and quenched with H₂O. The resulting mixture was extracted into EtOAc (2×400 mL), and the combined organic extracts were washed with brine (3×800 mL), dried over Na₂SO₄, filtered, and concentrated under reduced pressure. The residue was purified by silica gel column chromatography to afford (S)-2-(benzyl(2-hydroxy-1-phenylethyl)amino)acetonitrile (10.5 g, 90% yield) as an off-white solid. LCMS (ESI) m/z: [M+H] calcd for C₁₇H₁₈N₂O: 267.1; found 267.1.

Step 3. To a stirred solution of (S)-2-(benzyl(2-hydroxy-1-phenylethyl)amino)acetonitrile (10.5 g, 39.6 mmol) in DCM (120 mL) at 0° C. was added SOCl₂ (5.74 mL, 79.1 mmol) dropwise and the reaction mixture was heated to 50° C. After 3 h, the mixture was cooled to room temperature, neutralized to pH=7 with sat. aq. NaHCO₃, extracted into DCM (2×200 mL), dried over Na₂SO₄, filtered, concentrated under reduced pressure. The resulting residue was purified by silica gel column chromatography to afford (R)-2-(benzyl(2-chloro-2-phenylethyl)amino)acetonitrile (10.8 g, 96% yield) as an off-white solid. LCMS (ESI) m/z: [M+H] calcd for C₁₇H₁₇CIN₂: 285.1; found 285.0.

Step 4. To a stirred solution of (R)-2-(benzyl(2-chloro-2-phenylethyl)amino)acetonitrile (9.47 g, 33.3 mmol) in THE (950 mL) at −90° C. under an atmosphere of N₂ was added LiHMDS (66.5 mL, 66.5 mmol) dropwise After 1.5 h, the reaction was warmed to −10° C., quenched with sat. aq. NH₄Cl, extracted into EtOAc (2×500 mL), dried over Na₂SO₄, filtered, concentrated under reduced pressure, and purified by silica gel column chromatography to afford (2S,3S)-1-benzyl-3-phenylazetidine-2-carbonitrile (5.42 g, 66% yield) as a yellow oil.

Step 5. To a solution of (2S,3S)-1-benzyl-3-phenylazetidine-2-carbonitrile (5.20 g, 20.9 mmol) in CC4 (52 mL) stirred at 0° C. was added conc. HCl (52 mL, 1710 mmol) dropwise. The resulting mixture was stirred for 80° C. at room temperature then concentrated under reduced pressure to afford crude product ((2S,3S)-1-benzyl-3-phenylazetidine-2-carboxamide (6.3 g, crude) as a white solid, which used in the next step directly without further purification. LCMS (ESI) m/z: [M+H] calcd for C₁₇H₁₈N₂O: 267.1; found 267.1

Step 6. To a stirred solution of ((2S,3S)-1-benzyl-3-phenylazetidine-2-carboxamide (6.26 g, crude) in MeOH (70 mL) at 0° C. was added SOCl₂ (8.49 mL, 117 mmol) dropwise. The solution was heated to 50° C. and stirred for 12 hours, after which time the reaction mixture was concentrated under reduced pressure, basified to pH=9 with sat. aq. NaHCO₃, extracted into EtOAc (3×200 mL), dried over Na₂SO₄, filtered, and concentrated under reduced pressure to afford methyl (2S,3S)-1-benzyl-3-phenylazetidine-2-carboxylate (5.6 g, crude) as a brown oil, which was used in the next step without further purification. LCMS (ESI) m/z: [M+H] calcd for C₁₈H₁₉NO₂: 282.1; found 282.1.

Step 7. To a stirred solution of methyl (2S,3S)-1-benzyl-3-phenylazetidine-2-carboxylate (5.1 g, crude) and Boc₂O (7.91 g, 36.2 mmol) in MeOH (60 mL) was added 10 wt. % Pd/C (5.1 g). The resulting mixture was stirred for 2 h at room temperature under 1.5 atm of H₂, filtered, concentrated under reduced pressure, and purified by silica gel column chromatography to afford 1-(tert-butyl) 2-methyl (2S,3S)-3-phenylazetidine-1,2-dicarboxylate (5.30 g, 90% yield over 3 steps) as a colorless oil. LCMS (ESI) m/z: [2M+NH₄] calcd for C₁₆H₂₁NO₄: 600.3; found 600.3.

Step 8. To a stirred solution of NaIO₄ (77.8 g, 364 mmol) in ACN (60 mL) and H₂O (60 mL) was added RuCl₃ (9.04 mg, 0.034 mmol) in portions at room temperature. After 30 minutes, 1-(tert-butyl) 2-methyl (2S,3S)-3-phenylazetidine-1,2-dicarboxylate (5.3 g, 18.2 mmol) in ACN (90 mL) was added dropwise. After 24 h, the mixture was filtered, the filter cake washed with EtOAc (3×200 mL), and the filtrate concentrated under reduced pressure. The filtrate was diluted with water at 0° C. The mixture was then extracted with EtOAc (2×200 mL), washed with H₂O (3×400 mL), dried over Na₂SO₄, filtered, concentrated under reduced pressure, and purified by reversed phase column chromatography to afford (2S,3S)-1-(tert-butoxycarbonyl)-2-(methoxycarbonyl)azetidine-3-carboxylic acid (3.2 g, 68% yield) as a colorless oil. LCMS (ESI) m/z: [2M+Na] calcd for C₁₁H₁₇NO₆: 541.2; found 541.2.

Step 9. To a mixture of (2S,3S)-1-(tert-butoxycarbonyl)-2-(methoxycarbonyl)azetidine-3-carboxylic acid (1.0 g, 3.86 mmol) and benzyl (2S)-3-methyl-2-(methylamino)butanoate (1.11 g, 5.01 mmol) in DMF (10 mL) stirred at 0° C. were added 2,6-lutidine (2.07 g, 19.3 mmol) and CIP (2.15 g, 7.71 mmol) dropwise. After 30 min, the reaction was quenched with water at 0° C. The mixture was extracted with EtOAc (3×50 mL), washed with brine (3×150 mL), dried over Na₂SO₄, filtered, concentrated under reduced pressure, and purified by prep-TLC to afford 1-(tert-butyl) 2-methyl (2S,3S)-3-(((S)-1-(benzyloxy)-3-methyl-1-oxobutan-2-yl)(methyl)carbamoyl)azetidine-1,2-dicarboxylate (1.5 g, 84% yield) as a brown yellow oil. LCMS (ESI) m/z: [M+Na] calcd for C₂₄H₃₄N₂O₇: 485.2; found 485.3.

Step 10. To a stirred solution of 1-(tert-butyl) 2-methyl (2S,3S)-3-(((S)-1-(benzyloxy)-3-methyl-1-oxobutan-2-yl)(methyl)carbamoyl)azetidine-1,2-dicarboxylate (710 mg, 1.54 mmol) in toluene (7.1 mL) was added trimethyltin hydroxide (555 mg, 3.07 mmol) in portions at room temperature. The resulting mixture was heated to 100° C. After 30 min, the reaction was cooled to 0° C. and quenched with H₂O, extracted into EtOAc (3×20 mL), washed with H₂O (3×60 mL), dried over Na₂SO₄, filtered, concentrated under reduced pressure, and purified by prep-TLC to afford (2S,3S)-3-(((S)-1-(benzyloxy)-3-methyl-1-oxobutan-2-yl)(methyl)carbamoyl)-1-(tert-butoxycarbonyl)azetidine-2-carboxylic acid (395 mg, 57% yield) as a yellow oil. LCMS (ESI) m/z: [M+H] calcd for C₂₃H₃₂N₂O₇: 349.2; found 349.2.

Step 11. To a mixture of (2S,3S)-3-(((S)-1-(benzyloxy)-3-methyl-1-oxobutan-2-yl)(methyl)carbamoyl)-1-(tert-butoxycarbonyl)azetidine-2-carboxylic acid (116 mg, 1.15 mmol) in THE (5 mL) stirred at −15° C. was added 2-methylpropyl chloroformate (132 mg, 0.969 mmol) dropwise. After 30 min, a stirred solution of NaBH₄ (100 mg, 2.64 mmol) in THE (4 mL) and H₂O (1 mL) at 0° C. was added the above mixture dropwise. After 1 h, the reaction was quenched at 0° C. with water, extracted into EtOAc (3×20 mL), dried over Na₂SO₄, filtered, concentrated under reduced pressure, and purified by prep-TLC to afford tert-butyl (2S,3S)-3-(((S)-1-(benzyloxy)-3-methyl-1-oxobutan-2-yl)(methyl)carbamoyl)-2-(hydroxymethyl)azetidine-1-carboxylate (284 mg, 74% yield) as a colorless oil. LCMS (ESI) m/z: [M+Na] calcd for C₂₃H₃₄N₂O₆: 457.2; found 457.3.

Step 12. To a stirred mixture of tert-butyl (2S,3S)-3-(((S)-1-(benzyloxy)-3-methyl-1-oxobutan-2-yl)(methyl)carbamoyl)-2-(hydroxymethyl)azetidine-1-carboxylate (420 mg, 0.967 mmol), DMAP (11.8 mg, 0.097 mmol) and TEA (978 mg, 9.67 mmol) in DCM (5 mL) at 0° C. was added TsCl (552 mg, 2.90 mmol) dropwise. The reaction mixture was warmed to room temperature and stirred for 3 h, after which time it was cooled to 0° C. and quenched with H₂O. The resulting mixture was extracted into DCM (3×20 mL), dried over Na₂SO₄, filtered, concentrated under reduced pressure, and purified by prep-TLC to afford tert-butyl (2S,3S)-3-(((S)-1-(benzyloxy)-3-methyl-1-oxobutan-2-yl)(methyl)carbamoyl)-2-((tosyloxy)methyl)azetidine-1-carboxylate (461 mg, 81% yield) as a colorless oil. LCMS (ESI) m/z: [M+NH₄] calcd for C₃₀H₄₀N₂O₈S: 606.3; found 606.3.

Step 13. To a stirred solution of tert-butyl (2S,3S)-3-(((S)-1-(benzyloxy)-3-methyl-1-oxobutan-2-yl)(methyl)carbamoyl)-2-((tosyloxy)methyl)azetidine-1-carboxylate (451 mg, 0.766 mmol) in MeOH (5 mL) was added Pd(OH)₂/C (451 mg, 3.21 mmol). The resulting mixture was stirred for 1 h under 1.5 atm of H₂, filtered, and concentrated under reduced pressure to afford N-((2S,3S)-1-(tert-butoxycarbonyl)-2-((tosyloxy)methyl)azetidine-3-carbonyl)-N-methyl-L-valine (353 mg, crude) as an off-white solid, which was used in the next step without further purification. LCMS (ESI) m/z: [M+H] calcd for C₂₃H₃₄N₂O₈S: 499.2; found 499.3.

Step 14. To a mixture of (6³S,4S)-4-amino-1²-(5-(4-cyclopropylpiperazin-1-yl)-2-((S)-1-methoxyethyl)pyridin-3-yl)-1¹-ethyl-25-hydroxy-10,10-dimethyl-6¹,6²,6³,6⁴,6⁵,6⁶-hexahydro-1¹H-8-oxa-1(5,3)-indola-6(1,3)-pyridazina-2(1,3)-benzenacycloundecaphane-5,7-dione (400 mg, 0.524 mmol) and DIPEA (2.71 g, 21.0 mmol) in DMF (5 mL) stirred at −10° C. was added N-((2S,3S)-1-(tert-butoxycarbonyl)-2-((tosyloxy)methyl)azetidine-3-carbonyl)-N-methyl-L-valine (339 mg, crude) and PyBOP (354 mg, 0.681 mmol) dropwise. After 1 h, the reaction was quenched at −10° C. with H₂O. The resulting mixture was extracted into EtOAc (2×30 mL), washed with brine (3×60 mL), dried over Na₂SO₄, filtered, concentrated under reduced pressure, and purified by silica gel column chromatography to afford tert-butyl (2S,3S)-3-(((2S)-1-(((6³S,4S)-1²-(5-(4-cyclopropylpiperazin-1-yl)-2-((S)-1-methoxyethyl)pyridin-3-yl)-1¹-ethyl-2⁵-hydroxy-10,10-dimethyl-5,7-dioxo-6¹,6²,6³,6⁴,6⁵,6⁶-hexahydro-1¹H-8-oxa-1(5,3)-indola-6(1,3)-pyridazina- 2(1,3)-benzenacycloundecaphane-4-yl)amino)-3-methyl-1-oxobutan-2-yl)(methyl)carbamoyl)-2-((tosyloxy)methyl)azetidine-1-carboxylate (585 mg, 90% yield) as an off-white solid. LCMS (ESI) m/z: [M+H] calcd for C₆₇H₈₉N₉O₁₂S: 1244.6; found 1244.8.

Step 15. A stirred mixture of tert-butyl (2S,3S)-3-(((2S)-1-(((6³S,4S)-1²-(5-(4-cyclopropylpiperazin-1-yl)-2-((S)-1-methoxyethyl)pyridin-3-yl)-1¹-ethyl-2⁵-hydroxy-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)amino)-3-methyl-1-oxobutan-2-yl)(methyl)carbamoyl)-2-((tosyloxy)methyl)azetidine-1-carboxylate (575 mg, 0.462 mmol), K₂CO₃ (639 mg, 4.62 mmol), and KI (76.7 mg, 0.462 mmol) in DMF (58 mL) was heated to 80° C. under an atmosphere of N₂. After 2 h, the reaction was cooled to −10° C. and quenched with H₂O. The resulting mixture was extracted into EtOAc (3×80 mL), washed with brine (3×240 mL), dried over Na₂SO₄, filtered, concentrated under reduced pressure, and purified by prep-TLC to afford tert-butyl (2aS,5S,8S,14S,31aS)-20-(5-(4-cyclopropylpiperazin-1-yl)-2-((S)-1-methoxyethyl)pyridin-3-yl)-21-ethyl-5-isopropyl-4,18,18-trimethyl-3,6,9,15-tetraoxo-2a,3,4,5,6,7,8,9,12,13,14,15,17,18,19,21,31,31a-octadecahydro-11H-10,14-epimino-22,24-etheno-8,27-methano-25,29-(metheno)azeto[2,3-c]pyrrolo[3,4-v][1,18]dioxa[6,9,12]triazacyclotriacontine-1(2H)-carboxylate (243 mg, 49% yield) as an off-white solid. LCMS (ESI) m/z: [M+H] calcd for C₆₀H₈₁N₉O₉: 1072.6; found 1072.8.

Step 16. To a solution of tert-butyl (2aS,5S,8S,14S,31aS)-20-(5-(4-cyclopropylpiperazin-1-yl)-2-((S)-1-methoxyethyl)pyridin-3-yl)-21-ethyl-5-isopropyl-4,18,18-trimethyl-3,6,9,15-tetraoxo-2a,3,4,5,6,7,8,9,12,13,14,15,17,18,19,21,31,31a-octadecahydro-11H-10,14-epimino-22,24-etheno-8,27-methano-25,29-(metheno)azeto[2,3-c]pyrrolo[3,4-v][1,18]dioxa[6,9,12]triazacyclotriacontine-1(2H)-carboxylate (243 mg, 0.227 mmol) in DCM (2.5 mL) stirred at 0° C. was added TFA (500 μL) dropwise and the reaction was warmed to room temperature. After 1 h, the reaction was basified to pH=9 with sat. aq. NaHCO₃, extracted into DCM (3×50 mL), dried over Na₂SO₄, filtered, and concentrated under reduced pressure to afford (2aS,5S,8S,14S,31 aS)-20-(5-(4-cyclopropylpiperazin-1-yl)-2-((S)-1-methoxyethyl)pyridin-3-yl)-21-ethyl-5-isopropyl-4,18,18-trimethyl-2,2a,4,5,7,8,13,14,17,18,19,21,31,31a-tetradecahydro-11H-10,14-epimino-22,24-etheno-8,27-methano-25,29-(metheno)azeto[2,3-c]pyrrolo[3,4-v][1,18]dioxa[6,9,12]triazacyclotriacontine-3,6,9,15(1H,12H)-tetraone (220 mg, crude) as an off-white solid, which was used in the next step without further purification. LCMS (ESI) m/z: [M/2+H] calcd for C₅₅H₇₃N₉O₇: 486.8; found 487.1.

Step 17. To a mixture of (2aS,5S,8S,14S,31aS)-20-(5-(4-cyclopropylpiperazin-1-yl)-2-((S)-1-methoxyethyl)pyridin-3-yl)-21-ethyl-5-isopropyl-4,18,18-trimethyl-2,2a,4,5,7,8,13,14,17,18,19,21,31,31a-tetradecahydro-11H-10,14-epimino-22,24-etheno-8,27-methano-25,29-(metheno)azeto[2,3-c]pyrrolo[3,4-v][1,18]dioxa[6,9,12]triazacyclotriacontine-3,6,9,15(1H,12H)-tetraone (220 mg, crude) and 2-fluoro-2-methylpropanoic acid (48.0 mg, crude) in DMF (3 mL) stirred at 0° C. was added DIPEA (292 mg, 2.26 mmol) and COMU (145 mg, 0.339 mmol) dropwise. The reaction mixture was warmed to room temperature and stirred for 1 h, after which time it was cooled to 0° C., quenched with H₂O, extracted into EtOAc (3×20 mL), washed with brine (3×60 mL), dried over Na₂SO₄, filtered, concentrated under reduced pressure, and purified via reversed phase chromatography to afford (2aS,5S,8S,14S,31aS)-20-(5-(4-cyclopropylpiperazin-1-yl)-2-((S)-1-methoxyethyl)pyridin-3-yl)-21-ethyl-1-(2-fluoro-2-methylpropanoyl)-5-isopropyl-4,18,18-trimethyl-2,2a,4,5,7,8,13,14,17,18,19,21,31,31a-tetradecahydro-11H-10,14-epimino-22,24-etheno-8,27-methano-25,29-(metheno)azeto[2,3-c]pyrrolo[3,4-v][1,18]dioxa[6,9,12]triazacyclotriacontine-3,6,9,15(1H,12H)-tetraone (94.7 mg, 39% yield over 2 steps) as a white solid. LCMS (ESI) m/z: [M+H] calcd for C₅₉H₇₈FN₉O₈: 1060.6; found 1060.6. ¹H NMR (400 MHz, DMSO-d₆) δ 8.46 (d, J=2.8 Hz, 1H), 7.99 (s, 1H), 7.86 (d, J=8.2 Hz, 1H), 7.78-7.68 (m, 1H), 7.61-7.49 (m, 2H), 7.40-7.31 (m, 1H), 7.25 (d, J=2.8 Hz, 1H), 6.72-6.57 (m, 1H), 5.43 (d, J=12.2 Hz, 1H), 5.32-5.19 (m, 1H), 5.10-4.98 (m, 1H), 4.64 (d, J=11.1 Hz, 1H), 4.50-4.39 (m, 2H), 4.35-4.22 (m, 2H), 4.15-3.99 (m, 3H), 3.73-3.60 (m, 2H), 3.58-3.48 (m, 1H), 3.29-3.11 (m, 5H), 3.04 (s, 3H), 2.92-2.72 (m, 4H), 2.70-2.58 (s, 5H), 2.35 (s, 3H), 2.10-1.95 (m, 2H), 1.88-1.78 (m, 1H), 1.70-1.61 (m, 2H), 1.60-1.41 (m, 7H), 1.36 (d, J=6.2 Hz, 3H), 1.26-1.21 (m, 1H), 1.09-0.97 (m, 3H), 0.95-0.83 (m, 2H), 0.80 (d, J=6.4 Hz, 2H), 0.77-0.67 (m, 5H), 0.62-0.58 (m, 1H), 0.50-0.37 (m, 3H), 0.36-0.29 (m, 2H).

### Example A399. Synthesis of (3aS,6S,9S,15S,32aS)-21-(5-(1-cyclopropyl-4-methoxypiperidin-4-yl)-2-((S)-1-methoxyethyl)pyridin-3-yl)-22-ethyl-1-(2-fluoro-2-methylpropanoyl)-6-isopropyl-5,19,19-trimethyl-3,3a,5,6,8,9,14,15,18,19,20,22,32,32a-tetradecahydro-1H,12H-11,15-epimino-23,25-etheno-9,28-methano-26,30-(metheno)dipyrrolo[2,3-c:3′,4′-v][1,18]dioxa[6,9,12]triazacyclotriacontine-4,7,10,16(2H,13H)-tetraone

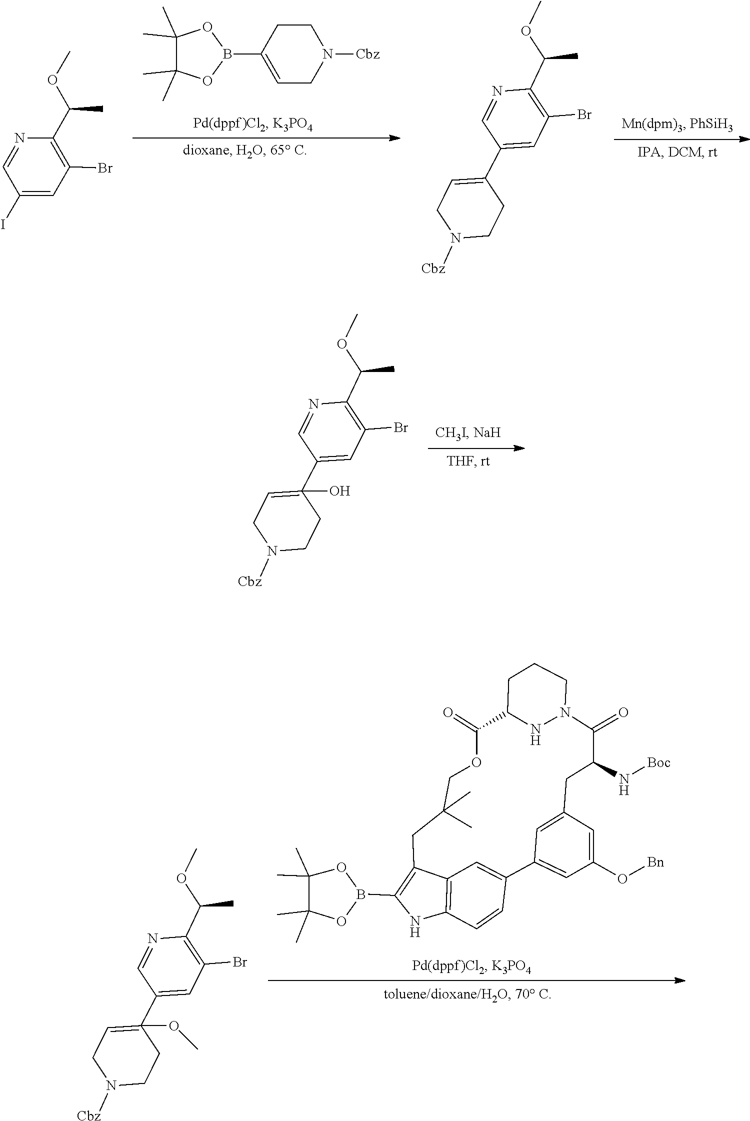

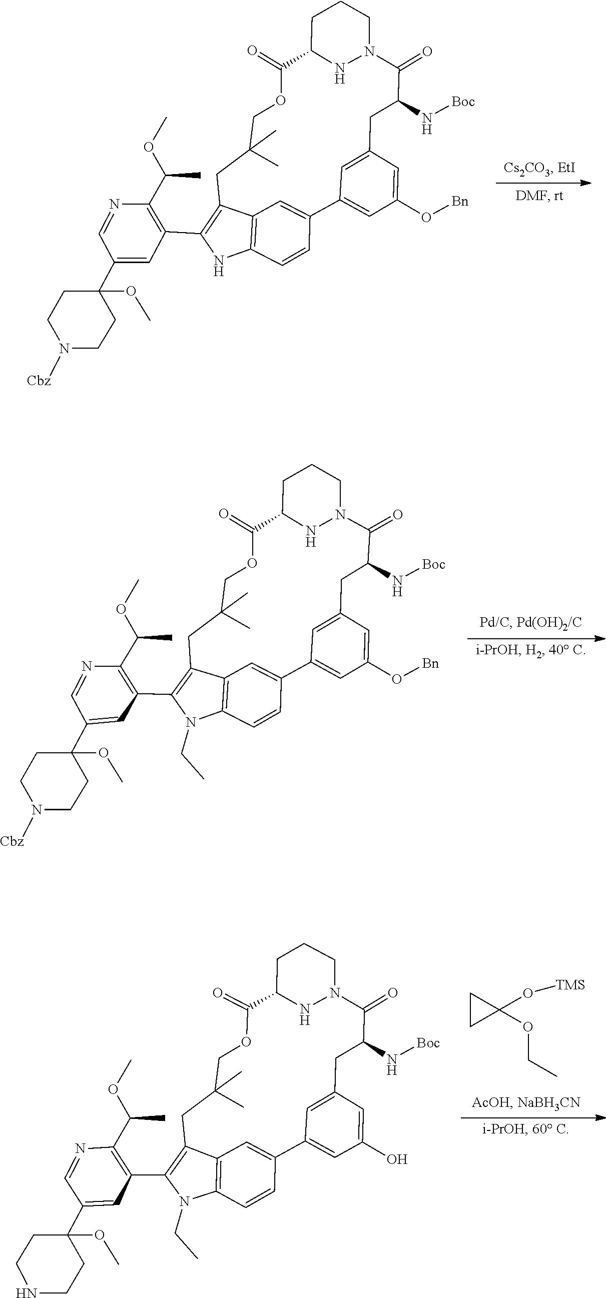

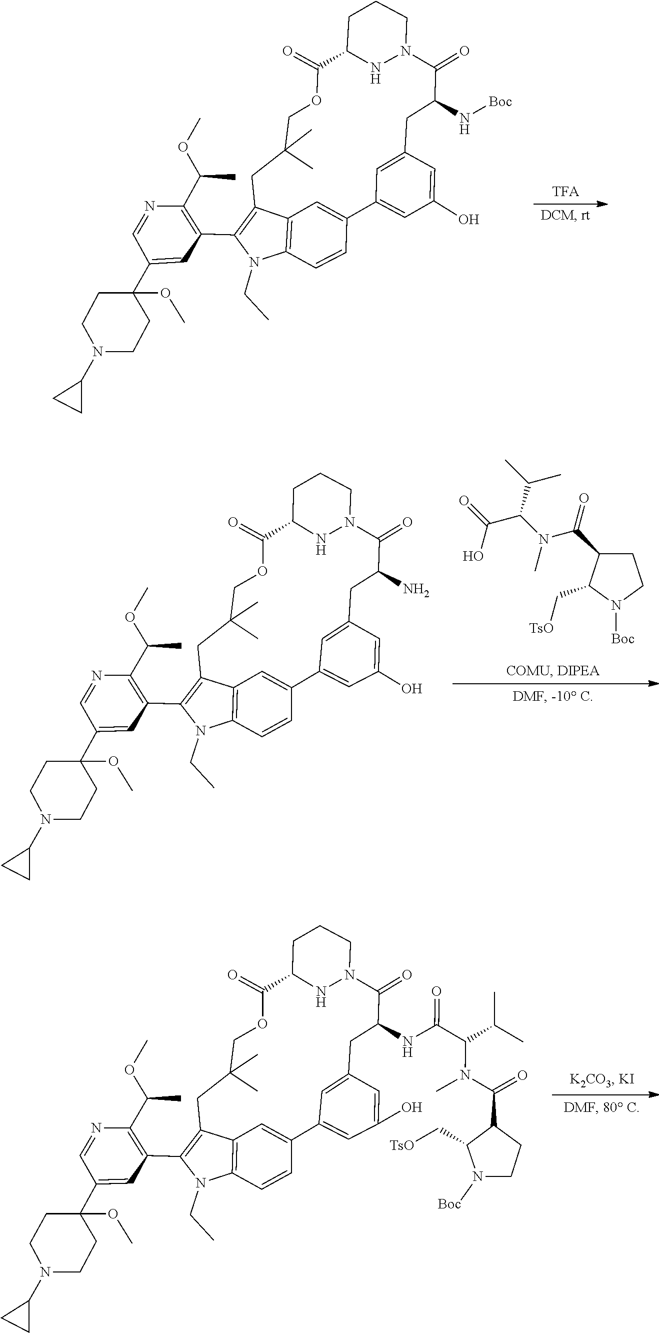

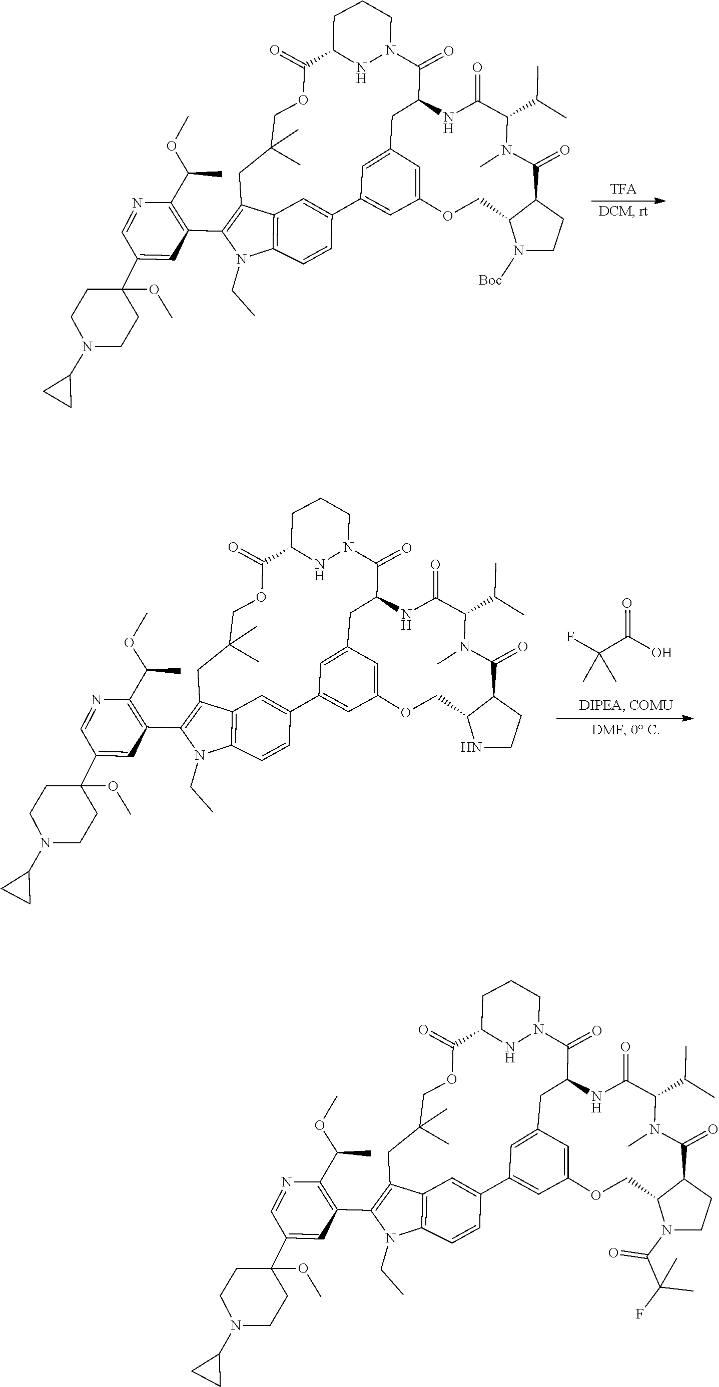

Step 1. To a stirred solution of (S)-3-bromo-5-iodo-2-(1-methoxyethyl)pyridine (10.0 g, 29.2 mmol) in 1,4-dioxane (90 mL) were added benzyl 4-(4,4,5,5-tetramethyl-1,3,2-dioxaborolan-2-yl)-3,6-dihydropyridine-1(2F)-carboxylate (10.0 g, 29.1 mmol), K₃PO₄ (18.6 g, 87.7 mmol), Pd(dppf)Cl₂ (2.14 g, 2.92 mmol), and H₂O (18 mL). The resulting mixture was stirred for 2 hours at 65° C. and was then treated with H₂O (200 mL) at 0° C. The aqueous mixture was extracted into EtOAc (3×300 mL), and the combined organic extracts were dried over Na₂SO₄, filtered, concentrated under reduced pressure, and purified by normal phase chromatography to afford benzyl (S)-5-bromo-6-(1-methoxyethyl)-3′,6′-dihydro-[3,4′-bipyridine]-1′(2′H)-carboxylate (13.0 g, 92% yield) as a brown oil. LCMS (ESI) m/z: [M+H] calcd for C₂₁H₂₃BrN₂O₃: 431.1; found 431.0.

Step 2. To a stirred solution of benzyl (S)-5-bromo-6-(1-methoxyethyl)-3′,6′-dihydro-[3,4′-bipyridine]-1′(2′H)-carboxylate (7.0 g, 16.2 mmol) in isopropanol (70 mL) at 0° C. was added DCM (10 mL), tris(2,2,6,6-tetramethyl-3,5-heptanedionato)manganese(III) (2.94 g, 4.87 mmol), and phenylsilane (10.54 g, 97.4 mmol). The resulting mixture was stirred overnight at 0° C. under an atmosphere of 02 and was quenched by the addition of sat. aq. Na₂S₂O₃ (200 mL) at 0° C. The aqueous layer was extracted into EtOAc (3×300 mL) and the combined organic extracts were dried over Na₂SO₄, filtered, concentrated under reduced pressure, and purified by reversed phase chromatography to afford benzyl (S)-4-(5-bromo-6-(1-methoxyethyl)pyridin-3-yl)-4-hydroxypiperidine-1-carboxylate (2.6 g, 32% yield) as a yellow oil. LCMS (ESI) m/z: [M+H] calcd for C₂₁H₂₅BrN₂O₄: 449.1; found 449.0.

Step 3. To a stirred solution of benzyl (S)-4-(5-bromo-6-(1-methoxyethyl)pyridin-3-yl)-4-hydroxypiperidine-1-carboxylate (2.6 g, 5.79 mmol) in THE (26 mL) at 0° C. was added NaH (0.42 g, 17.4 mmol). After 20 min, CH₃I (1.64 g, 11.6 mmol) was added dropwise, and the reaction was warmed to room temperature. After 2 h, the reaction was cooled to 0° C., quenched with sat. aq. NH₄Cl (100 mL), extracted into DCM (3×100 mL), dried over Na₂SO₄, filtered, concentrated under reduced pressure, and purified by normal phase chromatography to afford benzyl (S)-4-(5-bromo-6-(1-methoxyethyl)pyridin-3-yl)-4-methoxypiperidine-1-carboxylate (2.03 g, 68% yield) as a light yellow oil. LCMS (ESI) m/z: [M+H] calcd for C₂₂H₂₇BrN₂O₄: 465.1; found 465.0.

Step 4. To a stirred solution of benzyl (S)-4-(5-bromo-6-(1-methoxyethyl)pyridin-3-yl)-4-methoxypiperidine-1-carboxylate (742 mg, 1.60 mmol) in toluene (3 mL) was added tert-butyl ((63S,4S)-2⁵-(benzyloxy)-10,10-dimethyl-5,7-dioxo-1²-(4,4,5,5-tetramethyl-1,3,2-dioxaborolan-2-yl)-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)carbamate (1.40 g, 1.76 mmol), K₂CO₃ (443 mg, 3.20 mmol), Pd(dppf)Cl₂ (130 mg, 0.160 mmol), dioxane (5 mL), and H₂O (5 mL). The resulting solution was stirred for 2 h at 70° C. and was then cooled to 0° C., quenched with water (30 mL), extracted into EtOAc (3×30 mL), dried over Na₂SO₄, filtered, concentrated under reduced pressure, and purified by normal phase chromatography to afford benzyl 4-(5-((63S,4S)-25-(benzyloxy)-4-((tert-butoxycarbonyl)amino)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-12-yl)-6-((S)-1-methoxyethyl)pyridin-3-yl)-4-methoxypiperidine-1-carboxylate (1.43 g, 85% yield) as a white solid. LCMS (ESI) m/z: [M+H] calcd for C₆₁H₇₂N₆O₁₀: 1049.5; found 1049.3.

Step 5. To a stirred solution of benzyl 4-(5-((6³S,4S)-2⁵-(benzyloxy)-4-((tert-butoxycarbonyl)amino)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-12-yl)-6-((S)-1-methoxyethyl)pyridin-3-yl)-4-methoxypiperidine-1-carboxylate (1.42 g, 1.35 mmol) in DMF (15 mL) were added Cs₂CO₃ (1.32 mg, 4.06 mmol) and iodoethane (422 mg, 2.71 mmol). The resulting mixture was stirred for 3 h at rt and was then quenched by the addition water (50 mL) at 0° C. The aqueous mixture extracted into EtOAc (3×50 mL), washed with brine (3×100 mL), dried over Na₂SO₄, filtered, concentrated under reduced pressure, and purified by reversed phase chromatography to afford benzyl 4-(5-((6³S,4S)-2⁵-(benzyloxy)-4-((tert-butoxycarbonyl)amino)-1¹-ethyl-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-12-yl)-6-((S)-1-methoxyethyl)pyridin-3-yl)-4-methoxypiperidine-1-carboxylate (830 mg, 56% yield) as a light yellow solid. LCMS (ESI) m/z: [M+H] calcd for C₆₃H₇₆N₆O₁₀: 1077.6; found 1077.5.

Step 6. To a stirred solution of benzyl 4-(5-((6³S,4S)-2⁵-(benzyloxy)-4-((tert-butoxycarbonyl)amino)-1¹-ethyl-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-12-yl)-6-((S)-1-methoxyethyl)pyridin-3-yl)-4-methoxypiperidine-1-carboxylate (820 mg, 0.761 mmol) in IPA (8 mL) was added Pd/C (410 mg, 3.85 mmol) and Pd(OH)₂/C (410 mg, 2.92 mmol). The resulting solution was stirred overnight at 40° C. under an atmosphere of H₂. After stirring overnight, the mixture was filtered, the filter cake was washed with IPA (6×20 mL), and the filtrate was concentrated under reduced pressure to afford tert-butyl ((63S,4S)-11-ethyl-2⁵-hydroxy-1²-(2-((S)-1-methoxyethyl)-5-(4-methoxypiperidin-4-yl)pyridin-3-yl)-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)carbamate (429 mg, crude) as a yellow solid. LCMS (ESI) m/z: [M+H] calcd for C₄₈H₆₄N₆O₈: 853.5; found 853.5.

Step 7. To a stirred solution of tert-butyl ((63S,4S)-1¹-ethyl-2⁵-hydroxy-1²-(2-((S)-1-methoxyethyl)-5-(4-methoxypiperidin-4-yl)pyridin-3-yl)-10,10-dimethyl-5,7-dioxo-6¹,6²,6³,6⁴,6⁵,6⁶-hexahydro-1 ¹H-8-oxa-1(5,3)-indola-6(1,3)-pyridazina-2(1,3)-benzenacycloundecaphane-4-yl)carbamate (400 mg, crude) in IPA (5 mL) at 0° C. were added (1-ethoxycyclopropoxy)trimethylsilane (249 mg, 1.43 mmol), AcOH (57.1 mg, 0.952 mmol), and NaBH₃CN (89.7 mg, 1.43 mmol). The resulting solution was stirred for 5 h at 60° C. and was then quenched by the addition of sat. aq. NaHCO₃ (30 mL). The aqueous mixture was extracted into EtOAc (3×30 mL), dried over Na₂SO₄, filtered, concentrated under reduced pressure, and purified by normal phase chromatography to afford tert-butyl ((63S,4S)-12-(5-(1-cyclopropyl-4-methoxypiperidin-4-yl)-2-((S)-1-methoxyethyl)pyridin-3-yl)-1¹-ethyl-2⁵-hydroxy-10,10-dimethyl-5,7-dioxo-6¹,6²,6³,6⁴,6⁵,6⁶-hexahydro-1¹H-8-oxa-1 (5,3)-indola-6(1,3)-pyridazina-2(1,3)-benzenacycloundecaphane-4-yl)carbamate (290 mg, 46% yield over 2 steps) as a white solid. LCMS (ESI) m/z: [M+H] calcd for C₅₁H₆₈N₆O₈: 893.5; found 892.5.

Step 8. To a stirred solution of tert-butyl ((63S,4S)-1²-(5-(1-cyclopropyl-4-methoxypiperidin-4-yl)-2-((S)-1-methoxyethyl)pyridin-3-yl)-1¹-ethyl-2⁵-hydroxy-10,10-dimethyl-5,7-dioxo-6¹,6²,6³,6⁴,6⁵,6⁶-hexahydro-1¹H-8-oxa-1 (5,3)-indola-6(1,3)-pyridazina-2(1,3)-benzenacycloundecaphane-4-yl)carbamate (285 mg, 0.319 mmol) in DCM (1.5 mL) at 0° C. was added TFA (0.5 mL). The resulting solution was warmed to room temperature, stirred for 1 hour, then basified to pH 8 with sat. aq. NaHCO₃. The aqueous layer was extracted into DCM (3×20 mL), dried over Na₂SO₄, filtered, concentrated under reduced pressure, and re-crystallized to afford (6³S,4S)-4-amino-1²-(5-(1-cyclopropyl-4-methoxypiperidin-4-yl)-2-((S)-1-methoxyethyl)pyridin-3-yl)-1¹-ethyl-2⁵-hydroxy-10,10-dimethyl-6¹,6²,6³,6⁴,6⁵,6⁶-hexahydro-1¹H-8-oxa-1(5,3)-indola-6(1,3)-pyridazina-2(1,3)-benzenacycloundecaphane-5,7-dione (240 mg, 75% yield) as a white solid. LCMS (ESI) m/z: [M+H] calcd for C₄₆H₆₀N₆O₆: 793.5; found 793.4.

Step 9. To a stirred solution of (6³S,4S)-4-amino-1²-(5-(1-cyclopropyl-4-methoxypiperidin-4-yl)-2-((S)-1-methoxyethyl)pyridin-3-yl)-1¹-ethyl-2⁵-hydroxy-10,10-dimethyl-6¹,6²,6³,6⁴,6⁵,6⁶-hexahydro-1¹H-8-oxa-1(5,3)-indola-6(1,3)-pyridazina-2(1,3)-benzenacycloundecaphane-5,7-dione (237 mg, 0.299 mmol) in DMF (3 mL) at −10° C. were added DIPEA (386 mg, 2.99 mmol), N-((2S,3S)-1-(tert-butoxycarbonyl)-2-((tosyloxy)methyl)pyrrolidine-3-carbonyl)-N-methyl-L-valine (199 mg, 0.389 mmol), and COMU (166 mg, 0.389 mmol) in sequence. The resulting mixture was stirred at −10° C. for 1 h, warmed to 0° C., then quenched by the addition of water (20 mL). The aqueous mixture was extracted into EtOAc (3×20 mL), washed with brine (3×40 mL), dried over Na₂SO₄, filtered, concentrated under reduced pressure, and purified by reversed phase chromatography to afford tert-butyl (2S,3S)-3-(((2S)-1-(((6³S,4S)-1²-(5-(1-cyclopropyl-4-methoxypiperidin-4-yl)-2-((S)-1-methoxyethyl)pyridin-3-yl)-1¹-ethyl-2⁵-hydroxy-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)amino)-3-methyl-1-oxobutan-2-yl)(methyl)carbamoyl)-2-((tosyloxy)methyl)pyrrolidine-1-carboxylate (230 mg, 58% yield) as a white solid. LCMS (ESI) m/z: [M+Na] calcd for C₇₀H₉₄N₈O₁₃S: 1309.7; found 1309.6.

Step 10. To a stirred solution of tert-butyl (2S,3S)-3-(((2S)-1-(((6³S,4S)-1²-(5-(1-cyclopropyl-4-methoxypiperidin-4-yl)-2-((S)-1-methoxyethyl)pyridin-3-yl)-1¹-ethyl-2⁵-hydroxy-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)amino)-3-methyl-1-oxobutan-2-yl)(methyl)carbamoyl)-2-((tosyloxy)methyl)pyrrolidine-1-carboxylate (225 mg, 0.193 mmol) in DMF (20 mL) at 0° C. were added K₂CO₃ (266 mg, 1.93 mmol) and KI (32 mg, 0.193 mmol). The reaction mixture was stirred for 1 h at 80° C., cooled to 0° C., and quenched with water (50 mL). The aqueous mixture was extracted into EtOAc (3×50 mL), washed with brine (3×100 mL), dried over Na₂SO₄, filtered, concentrated under reduced pressure, and purified by reversed phase chromatography to afford tert-butyl (3aS,6S,9S,15S,32aS)-21-(5-(1-cyclopropyl-4-methoxypiperidin-4-yl)-2-((S)-1-methoxyethyl)pyridin-3-yl)-22-ethyl-6-isopropyl-5,19,19-trimethyl-4,7,10,16-tetraoxo-2,3,3a,4,5,6,7,8,9,10,13,14,15,16,18,19,20,22,32,32a-icosahydro-1H,12H-11,15-epimino-23,25-etheno-9,28-methano-26,30-(metheno)dipyrrolo[2,3-c:3′,4′-v][1,18]dioxa[6,9,12]triazacyclotriacontine-1-carboxylate (160 mg, 80% yield) as a white solid. LCMS (ESI) m/z: [M+H] calcd for C₆₃H₈₆N₈O₁₀: 1115.7; found 1116.5.

Step 11. To a stirred solution of tert-butyl (3aS,6S,9S,15S,32aS)-21-(5-(1-cyclopropyl-4-methoxypiperidin-4-yl)-2-((S)-1-methoxyethyl)pyridin-3-yl)-22-ethyl-6-isopropyl-5,19,19-trimethyl-4,7,10,16-tetraoxo-2,3,3a,4,5,6,7,8,9,10,13,14,15,16,18,19,20,22,32,32a-icosahydro-1H,12H-11,15-epimino-23,25-etheno-9,28-methano-26,30-(metheno)dipyrrolo[2,3-c:3′,4′-v][1,18]dioxa[6,9,12]triazacyclotriacontine-1-carboxylate (145 mg, 0.130 mmol) in DCM (1.5 mL) at 0° C. was added TFA (0.3 mL). The reaction was allowed to warm to room temperature. After 1 h, the reaction was cooled to 0° C., quenched with water (10 mL), basified to pH 8 with sat. aq. NaHCO₃, extracted into DCM (3×20 mL), dried over Na₂SO₄, filtered, and concentrated under reduced pressure to afford (3aS,6S,9S,15S,32aS)-21-(5-(1-cyclopropyl-4-methoxypiperidin-4-yl)-2-((S)-1-methoxyethyl)pyridin-3-yl)-22-ethyl-6-isopropyl-5,19,19-trimethyl-3,3a,5,6,8,9,14,15,18,19,20,22,32,32a-tetradecahydro-1H,12H-11,15-epimino-23,25-etheno-9,28-methano-26,30-(metheno)dipyrrolo[2,3-c:3′,4′-v][1,18]dioxa[6,9,12]triazacyclotriacontine-4,7,10,16(2H,13H)-tetraone (170 mg, crude) as a white solid. LCMS (ESI) m/z: [M/2+H] calcd for C₅₈H₇₈N₈O₈: 508.3; found 508.5.

Step 12. To a stirring solution of (3aS,6S,9S,15S,32aS)-21-(5-(1-cyclopropyl-4-methoxypiperidin-4-yl)-2-((S)-1-methoxyethyl)pyridin-3-yl)-22-ethyl-6-isopropyl-5,19,19-trimethyl-3,3a,5,6,8,9,14,15,18,19,20,22,32,32a-tetradecahydro-1H,12H-11,15-epimino-23,25-etheno-9,28-methano-26,30-(metheno)dipyrrolo[2,3-c:3′,4′-v][1,18]dioxa[6,9,12]triazacyclotriacontine-4,7,10,16(2H,13H)-tetraone (165 mg, crude) in DMF (2 mL) at 0° C. were added DIPEA (206 mg, 1.60 mmol), 2-fluoro-2-methylpropanoic acid (33.9 mg, 0.320 mmol), and COMU (137 mg, 0.320 mmol). The reaction mixture was stirred for 1 h at 0° C. and was then quenched by the addition of water (10 mL). The aqueous mixture was extracted into EtOAc (3×20 mL), washed with brine (3×40 mL), dried over Na₂SO₄, filtered, concentrated under reduced pressure, and purified by reversed phase chromatography to afford (3aS,6S,9S,15S,32aS)-21-(5-(1-cyclopropyl-4-methoxypiperidin-4-yl)-2-((S)-1-methoxyethyl)pyridin-3-yl)-22-ethyl-1-(2-fluoro-2-methylpropanoyl)-6-isopropyl-5,19,19-trimethyl-3,3a,5,6,8,9,14,15,18,19,20,22,32,32a-tetradecahydro-1H,12H-11,15-epimino-23,25-etheno-9,28-methano-26,30-(metheno)dipyrrolo[2,3-c:3′,4′-v][1,18]dioxa[6,9,12]triazacyclotriacontine-4,7,10,16(2H,13H)-tetraone (29 mg, 21% yield over 2 steps) as a white solid. LCMS (ESI) m/z: [M+H] calcd for C₆₂H₈₃FN₈O₉: 1103.6; found 1103.6. ¹H NMR (400 MHz, DMSO-d₆) δ 8.78 (s, 1H), 8.31-7.95 (m, 1H), 7.94-7.65 (m, 2H), 7.64-7.53 (m, 1H), 7.52-7.43 (m, 1H), 7.42-7.38 (m, 1H), 7.37-7.00 (m, 1H), 6.92-6.51 (m, 1H), 5.51-4.80 (m, 1H), 4.70-4.62 (m, 1H), 4.60-4.35 (m, 2H), 4.34-4.15 (m, 2H), 4.05-3.92 (m, 2H), 3.81-3.60 (m, 3H), 3.50-3.41 (m, 1H), 3.32-3.25 (m, 3H), 3.10-3.02 (s, 1H), 3.01-2.70 (m, 10H), 2.68-2.52 (m, 2H), 2.25-2.16 (m, 3H), 2.14-1.81 (m, 7H), 1.80-1.33 (m, 14H), 1.31-1.17 (m, 2H), 1.16-0.85 (m, 5H), 0.83-0.72 (m, 4H), 0.70-0.52 (m, 5H), 0.50-0.10 (m, 6H).

### Example A402. Synthesis of (3aS,6S,9S,15S,32aS)-21-(5-(4-cyclopropylpiperazin-1-yl)-2-((S)-1-methoxyethyl)pyridin-3-yl)-22-ethyl-6-isopropyl-5,19,19-trimethyl-1-(oxazol-5-yl)-3,3a,5,6,8,9,14,15,18,19,20,22,32,32a-tetradecahydro-1H,12H-11,15-epimino-23,25-etheno-9,28-methano-26,30-(metheno)dipyrrolo[2,3-c:3′,4′-v][1,18]dioxa[6,9,12]triazacyclotriacontine-4,7,10,16(2H,13H)-tetraone

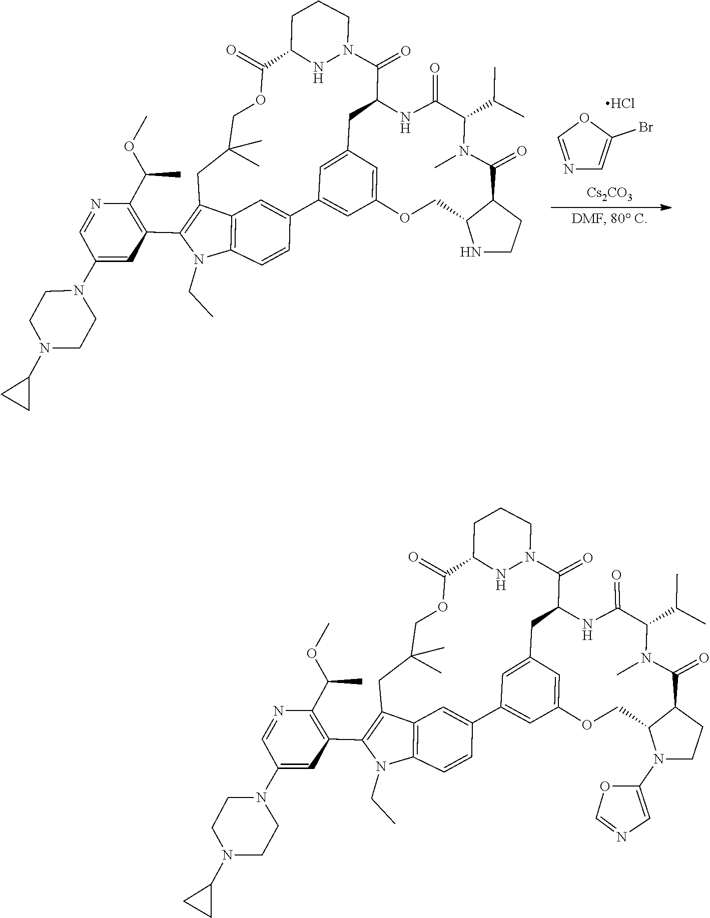

Step 1. To a stirred solution of (3aS,6S,9S,15S,32aS)-21-(5-(4-cyclopropylpiperazin-1-yl)-2-((S)-1-methoxyethyl)pyridin-3-yl)-22-ethyl-6-isopropyl-5,19,19-trimethyl-3,3a,5,6,8,9,14,15,18,19,20,22,32,32a-tetradecahydro-1H,12H-11,15-epimino-23,25-etheno-9,28-methano-26,30-(metheno)dipyrrolo[2,3-c:3′,4′-v][1,18]dioxa[6,9,12]triazacyclotriacontine-4,7,10,16(2H,13H)-tetraone (100 mg, 0.101 mmol) and Cs₂CO₃ (132 mg, 0.404 mmol) in DMF (2 mL) was added 5-bromooxazole (60.0 mg, 0.404 mmol) at room temperature. The reaction mixture was stirred for 6 h at 80° C. and was then quenched by the addition of H₂O at 0° C. The aqueous mixture was extracted with EtOAc (3×10 mL), washed with brine (3×30 mL), dried over Na₂SO₄, filtered, and concentrated under reduced pressure. The crude product was purified by reversed phase chromatography to give (3aS,6S,9S,15S,32aS)-21-(5-(4-cyclopropylpiperazin-1-yl)-2-((S)-1-methoxyethyl)pyridin-3-yl)-22-ethyl-6-isopropyl-5,19,19-trimethyl-1-(oxazol-5-yl)-3,3a,5,6,8,9,14,15,18,19,20,22,32,32a-tetradecahydro-1H,12H-11,15-epimino-23,25-etheno-9,28-methano-26,30-(metheno)dipyrrolo[2,3-c:3′,4′-v][1,18]dioxa[6,9,12]triazacyclotriacontine-4,7,10,16(2H,13H)-tetraone (5.3 mg, 4.6% yield) as a white solid. LCMS (ESI) m/z: [M+H] calcd for C₅₉H₇₆N₁₀O₈: 1053.6; found 1053.6; ¹H NMR (400 MHz, DMSO-d₆) δ 8.46 (d, J=2.8 Hz, 1H), 8.16 (d, J=8.0 Hz, 1H), 8.01 (s, 1H), 7.77-7.64 (m, 1H), 7.62-7.56 (m, 1H), 7.52-7.38 (m, 2H), 7.24-7.17 (m, 2H), 6.85-6.77 (m, 1H), 6.24-5.90 (m, 1H), 5.37 (d, J=12.3 Hz, 1H), 5.30-5.24 (m, 1H), 5.17-5.06 (m, 1H), 4.84-4.35 (m, 5H), 4.22-3.84 (m, 4H), 3.76-3.48 (m, 4H), 3.25-3.02 (m, 6H), 2.97-2.81 (m, 2H), 2.80-2.69 (m, 3H), 2.68-2.67 (m, 1H), 2.58-2.49 (m, 4H), 2.45-2.31 (m, 2H), 2.29-2.25 (m, 1H), 2.05-1.84 (m, 2H), 1.78-1.70 (m, 1H), 1.68-1.36 (m, 4H), 1.37-1.19 (m, 3H), 1.16-1.05 (m, 1H), 1.02-0.94 (m, 2H), 0.89-0.85 (d, J=6.4 Hz, 1H), 0.84-0.81 (m, 1H), 0.80-0.74 (m, 2H), 0.74-0.64 (m, 3H), 0.63-0.55 (m, 2H), 0.53-0.48 (m, 2H) 0.49-0.37 (m, 3H), 0.38-0.28 (m, 2H).

### Example A410. Synthesis of (3aS,6S,9S,15S,30S,32aS)-21-(5-(4-cyclopropylpiperazin-1-yl)-2-((S)-1-methoxyethyl)pyridin-3-yl)-22-ethyl-1-(2-fluoro-2-methylpropanoyl)-6-isopropyl-5,19,19-trimethyl-3,3a,5,6,8,9,14,15,18,19,20,22,29,30,32,32a-hexadecahydro-1H,12H,27H-11,15-epimino-23,25-etheno-9,28-methano-26,30-(metheno)dipyrrolo[3,2-b₁:3′,4′-i][1,14]dioxa[4,20,23,26]tetraazacyclotriacontine-4,7,10,16(2H,13H)-tetraone

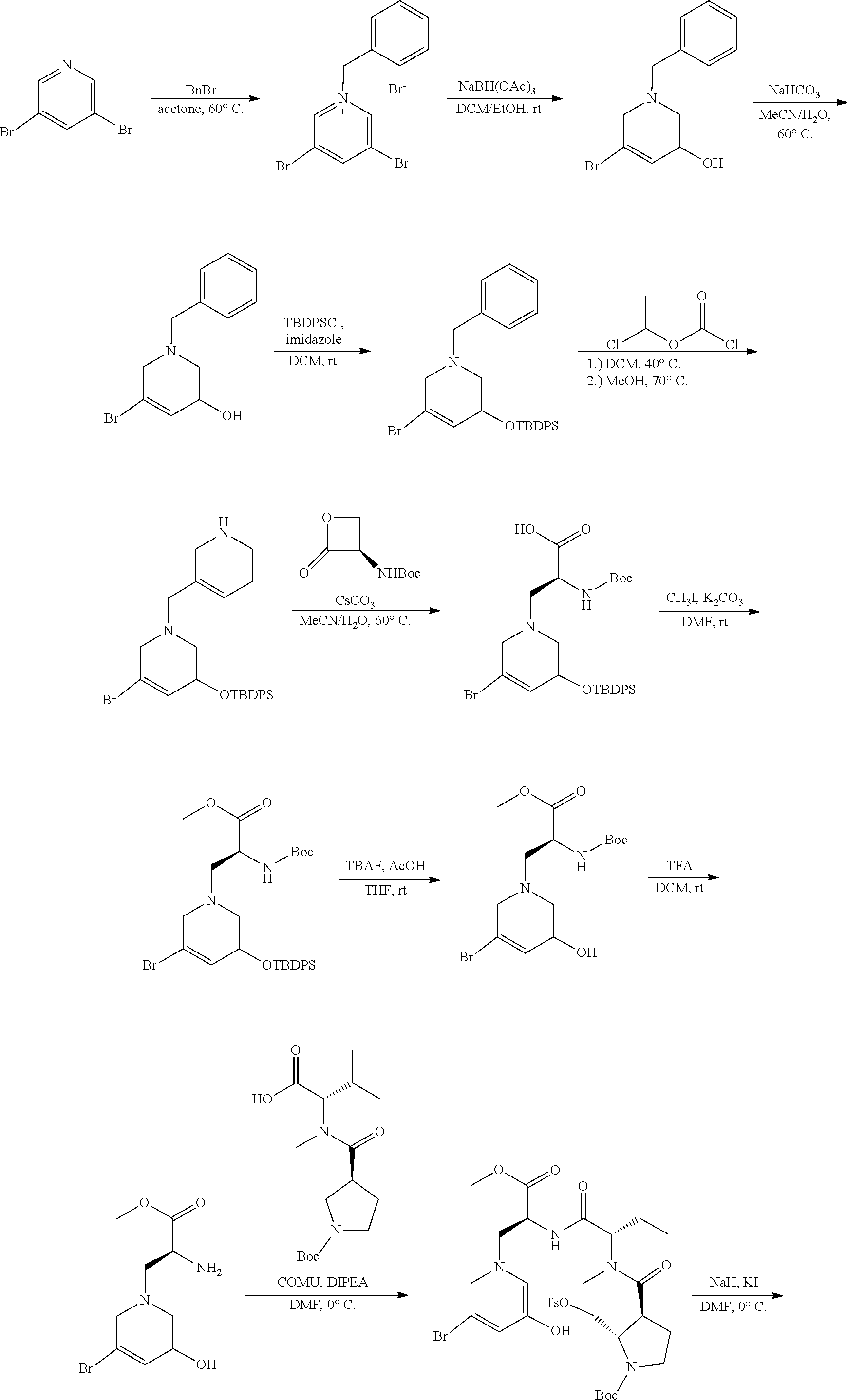

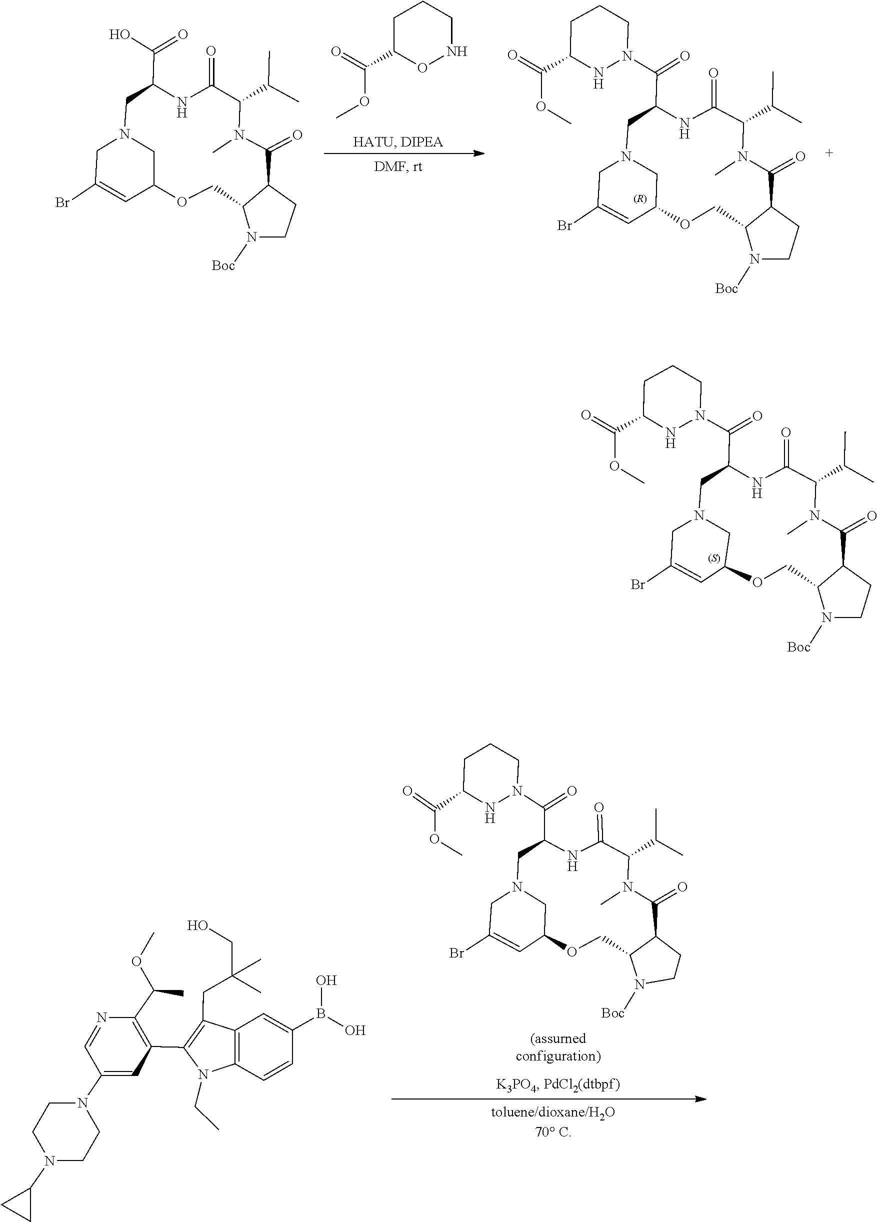

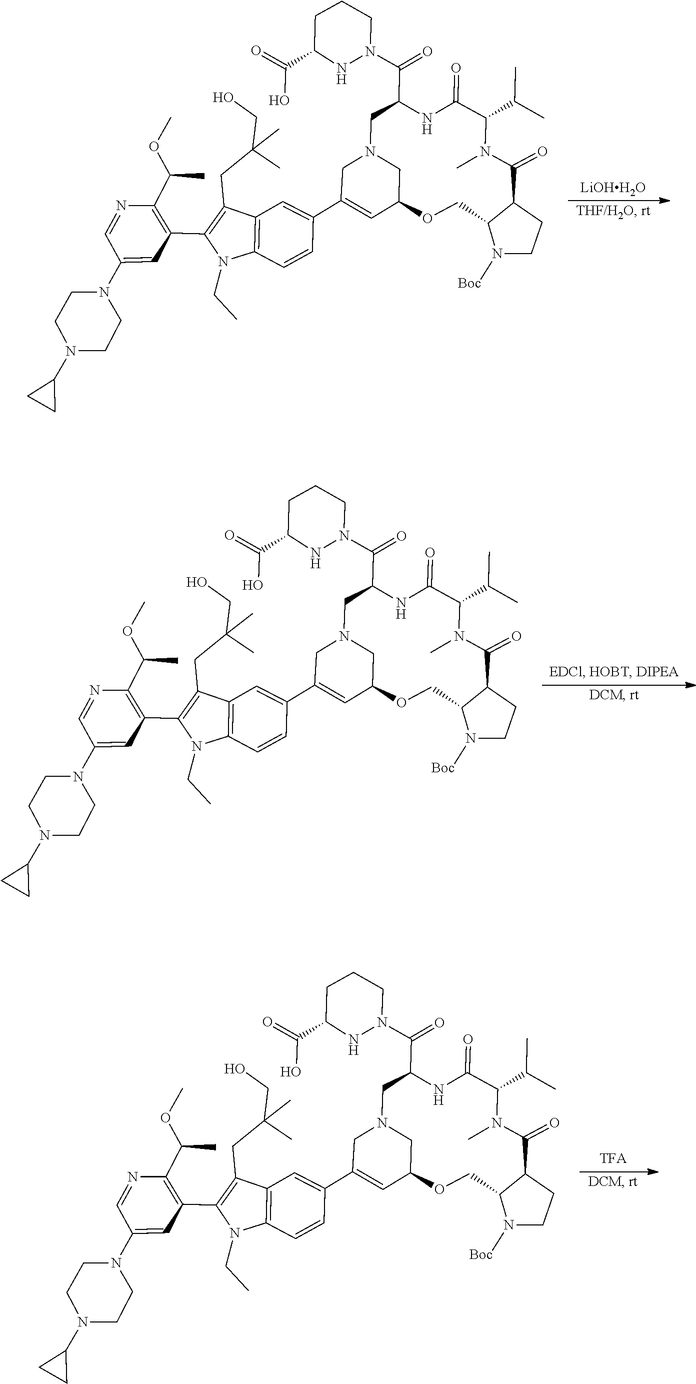

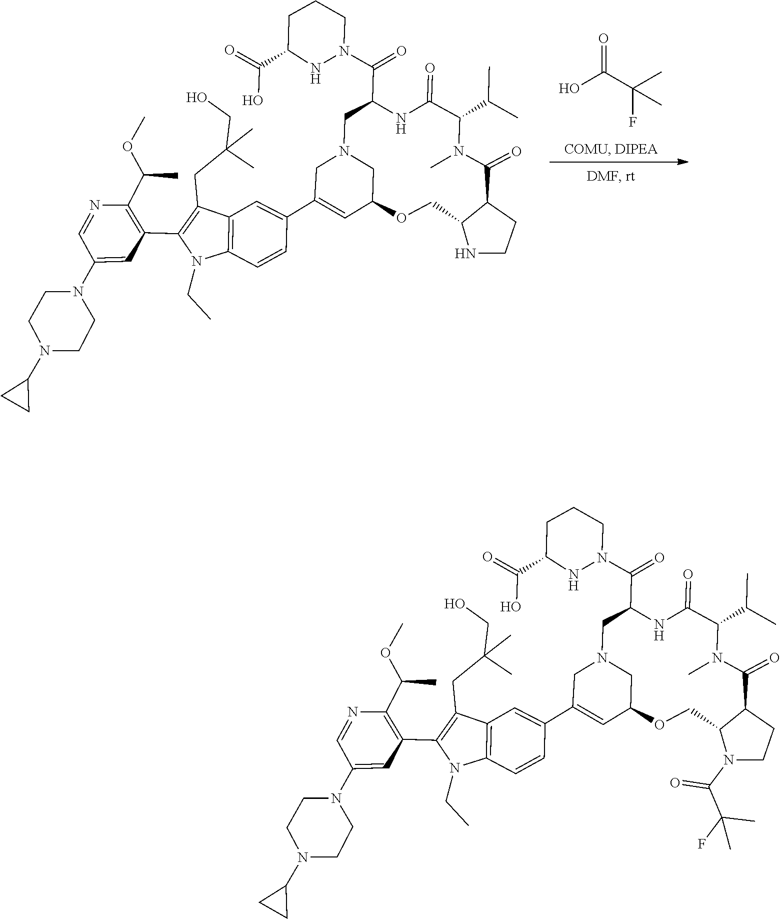

Step 1. To a solution of 3,5-dibromopyridine (10 g, 42.2 mmol) in acetone (100 mL) at 0° C. was added benzyl bromide (10.8 g, 63.3 mmol). The resulting mixture was stirred overnight at 60° C., filtered, and the collected solids washed with acetone (3×100 mL) to afford 1-benzyl-3,5-dibromopyridin-1-ium bromide (7.8 g, crude) as a yellow solid. The crude material was taken directly to the next step without further purification. LCMS (ESI) m/z: [M] calcd for C₁₂H₁₀Br₃N: 325.9; found 326.0.

Step 2. To a solution of 1-benzyl-3,5-dibromopyridin-1-ium bromide (7.8 g, crude) in DCM (80 mL) and EtOH (4.0 mL) at 0° C. was added NaBH(OAc)₃ (25.2 g, 119 mmol). The resulting mixture was stirred for 3 h at room temperature then quenched at 0° C. by the addition of H₂O. The aqueous layer was extracted with DCM (3×100 mL) and the combined organic extracts were dried over Na₂SO₄, filtered, and concentrated under reduced pressure. Purification by normal phase chromatography afforded 1-benzyl-3,5-dibromo-1,2,3,6-tetrahydropyridine (4.70 g, 34% yield over 2 steps) as a yellow oil. LCMS (ESI) m/z: [M+H] calcd for C₁₂H₁₃Br₂N: 330.0; found 330.0.

Step 3. To a solution of 1-benzyl-3,5-dibromo-1,2,3,6-tetrahydropyridine (4.7 g, 14.2 mmol) in MeCN (30 mL) and H₂O (30 mL) at 0° C. was added NaHCO₃ (5.96 g, 70.9 mmol). The resulting mixture was stirred for 3 h at 60° C. and was then diluted at 0° C. by the addition of H₂O. The aqueous layer was extracted with EtOAc (3×50 mL), and the combined organic extracts were dried over Na₂SO₄, filtered, and concentrated under reduced pressure. Purification by normal phase chromatography afforded 1-benzyl-5-bromo-1,2,3,6-tetrahydropyridin-3-ol (2.3 g, 54% yield) as a yellow solid. LCMS (ESI) m/z: [M+H] calcd for C₁₂H₁₄BrNO: 268.0; found 267.9.

Step 4. To a solution of 1-benzyl-5-bromo-1,2,3,6-tetrahydropyridin-3-ol (5.3 g, 19.8 mmol) and imidazole (2.96 g, 43.5 mmol) in DCM (53 mL) at 0° C. was added TBDPSCI (8.15 g, 29.6 mmol). The resulting mixture was stirred overnight at room temperature then quenched at 0° C. by the addition of H₂O. The aqueous layer was extracted with DCM (3×50 mL) and combined organic extracts were washed with brine (3×50 mL), dried over Na₂SO₄, filtered, and concentrated under reduced pressure. Purification by normal phase chromatography afforded 1-benzyl-5-bromo-3-((tert-butyldiphenylsilyl)oxy)-1,2,3,6-tetrahydropyridine (11.2 g, 78% yield) as a yellow oil. LCMS (ESI) m/z: [M+H] calcd for C₂₈H₃₂BrNOSi: 506.2; found 506.1.

Step 5. To a solution of 1-benzyl-5-bromo-3-((tert-butyldiphenylsilyl)oxy)-1,2,3,6-tetrahydropyridine (11.1 g, 21.9 mmol) in DCM (550 mL) at 0° C. was added 1-chloroethyl chloroformate (12.5 g, 87.7 mmol). The resulting mixture was stirred for 4 h at 40° C. and was then concentrated under reduced pressure. The concentrate was diluted by the addition of MeOH (550 mL) and the resulting mixture was stirred for 2 h at 70° C. then was concentrated under reduced pressure. Purification by reverse phase chromatography afforded 5-bromo-3-((tert-butyldiphenylsilyl)oxy)-1,2,3,6-tetrahydropyridine (6.2 g, 63% yield) as a yellow oil. LCMS (ESI) m/z: [M+H] calcd for C₂₁H₂₆BrNOSi: 416.1; found 416.1.

Step 6. To a solution of 5-bromo-3-((tert-butyldiphenylsilyl)oxy)-1,2,3,6-tetrahydropyridine (6.3 g, 15.1 mmol) and Cs₂CO₃ (19.7 g, 60.5 mmol) in MeCN (60 mL) and H₂O (20 mL) was added tert-butyl (R)-(2-oxooxetan-3-yl)carbamate (4.25 g, 22.7 mmol). The resulting mixture was stirred for 4 h at 60° C. then quenched at 0° C. by the addition of H₂O. The aqueous mixture was extracted with EtOAc (3×100 mL), and the combined organic extracts dried over Na₂SO₄, filtered, and concentrated under reduced pressure. Purification by reverse phase chromatography afforded (2S)-3-(5-bromo-3-((tert-butyldiphenylsilyl)oxy)-3,6-dihydropyridin-1(2F)-yl)-2-((tert-butoxycarbonyl)amino)propanoic acid (6.6 g, 67% yield) as a white solid. LCMS (ESI) m/z: [M+H] calcd for C₂₉H₃₉BrN₂O₅Si: 603.2; found 603.0.

Step 7. To a solution of (2S)-3-(5-bromo-3-((tert-butyldiphenylsilyl)oxy)-3,6-dihydropyridin-1(2F)-yl)-2-((tert-butoxycarbonyl)amino)propanoic acid (6.1 g, 10.1 mmol) and K₂CO₃ (2.79 g, 20.2 mmol) in DMF (61 mL) at 0° C. was added methyl iodide (2.15 g, 15.1 mmol). The resulting mixture was stirred for 2 h at room temperature and was then quenched at 0° C. by the addition of H₂O. The aqueous mixture was extracted with EtOAc (3×10 mL), and the combined organic extracts were washed with brine (3×10 mL), dried over Na₂SO₄, filtered, and concentrated under reduced pressure. Purification by normal phase chromatography afforded methyl (2S)-3-(5-bromo-3-((tert-butyldiphenylsilyl)oxy)-3,6-dihydropyridin-1(2F)-yl)-2-((tert-butoxycarbonyl)amino)propanoate (4.4 g, 63% yield) as a yellow oil. LCMS (ESI) m/z: [M+H] calcd for C₃₀H₄₁BrN₂O₅Si: 617.2; found 617.1.

Step 8. To a solution of methyl (2S)-3-(5-bromo-3-((tert-butyldiphenylsilyl)oxy)-3,6-dihydropyridin-1(2F)-yl)-2-((tert-butoxycarbony)amino)propanoate (4.4 g, 7.12 mmol) and acetic acid (42.8 mg, 0.712 mmol) in THE (30 mL) at 0° C. was added TBAF (9.31 g, 35.6 mmol). The resulting mixture was stirred for 4 h at room temperature and was then quenched at 0° C. by the addition of H₂O. The aqueous mixture was extracted with EtOAc (3×10 mL) and the combined organic extracts were washed with brine (3×10 mL), dried over Na₂SO₄, filtered, and concentrated under reduced pressure. Purification by reversed phase chromatography afforded methyl (2S)-3-(5-bromo-3-hydroxy-3,6-dihydropyridin-1(2F)-yl)-2-((tert-butoxycarbonyl)amino)propanoate (1.4 g, 48% yield) as a yellow oil. LCMS (ESI) m/z: [M+H] calcd for C₁₄H₂₃BrN₂O₅: 379.1; found 379.0.

Step 9. To a solution of methyl (2S)-3-(5-bromo-3-hydroxy-3,6-dihydropyridin-1(2F)-yl)-2-((tert-butoxycarbonyl)amino)propanoate (1 g, 2.6 mmol) in DCM (12 mL) at 0° C. was added TFA (4.0 mL). The resulting mixture was stirred for 1 h at room temperature and was then basified to pH=8 with sat. aq. NaHCO₃. The aqueous layer was extracted with 3:1 vol. DCM/IPA (3×15 mL), and the combined organic extracts were dried over Na₂SO₄, filtered, and concentrated under reduced pressure to afford methyl (2S)-2-amino-3-(5-bromo-3-hydroxy-3,6-dihydropyridin-1(2F)-yl)propanoate (750 mg, crude) which was taken directly to the next step without further purification. LCMS (ESI) m/z: [M+H] calcd for C₉H₁₅BrN₂O₃: 279.0; found 278.9.

Step 10. To a solution of methyl (2S)-2-amino-3-(5-bromo-3-hydroxy-3,6-dihydropyridin-1(2F)-yl)propanoate (750 mg, crude), DIPEA (13.9 g, 107 mmol) and (2S)-2-{1-[(2S,3S)-1-(tert-butoxycarbonyl)-2-{[(4-methylbenzenesulfonyl)oxy]methyl}pyrrolidin-3-yl]-N-methylformamido}-3-methylbutanoic acid (1.79 g, crude) in DMF (10 mL) at 0° C. was added COMU (1.50 g, 3.49 mmol). The resulting mixture was stirred for 1 h at 0° C. and was then quenched by the addition of H₂O. The aqueous mixture was extracted with EtOAc (3×20 mL), and the combined organic extracts were washed with brine (3×10 mL), dried over Na₂SO₄, filtered, and concentrated under reduced pressure. The residue was purified by reversed phase chromatography afforded tert-butyl (2S,3S)-3-(((2S)-1-(((2S)-3-(5-bromo-3-hydroxy-3,6-dihydropyridin-1(2H)-yl)-1-methoxy-1-oxopropan-2-yl)amino)-3-methyl-1-oxobutan-2-yl)(methyl)carbamoyl)-2-((tosyloxy)methyl)pyrrolidine-1-carboxylate (967 mg, 48% yield over 2 steps) as a yellow oil. LCMS (ESI) m/z: [M+H] calcd for C₃₃H₄₉BrN₄O₁₀S: 773.3; found 773.0.

Step 11. To a solution of tert-butyl (2S,3S)-3-(((2S)-1-(((2S)-3-(5-bromo-3-hydroxy-3,6-dihydropyridin-1 (2H)-yl)-1-methoxy-1-oxopropan-2-yl)amino)-3-methyl-1-oxobutan-2-yl)(methyl)carbamoyl)-2-((tosyloxy)methyl)pyrrolidine-1-carboxylate (630 mg, 0.814 mmol) and KI (135 mg, 0.814 mmol) in DMF (6.0 mL) at 0° C. was added NaH (58.6 mg, 2.44 mmol). The resulting mixture was stirred overnight at 0° C., quenched by the addition of H₂O, and acidified to pH=5 with 1M HCl. The aqueous mixture was extracted with 3:1 vol. DCM/IPA (3×50 mL), and the combined organic extracts were washed with brine (3×60 mL), dried over Na₂SO₄, filtered, and under reduced pressure to afford (3aS,6S,9S,17aS)-13-bromo-1-(tert-butoxycarbonyl)-6-isopropyl-5-methyl-4,7-dioxo-2,3,3a,4,5,6,7,8,9,10,12,15,17,17a-tetradecahydro-1H-11,15-methanopyrrolo[2,3-c][1]oxa[6,9,12]triazacyclohexadecine-9-carboxylic acid (620 mg, crude). This crude material was taken directly to the next step without further purification. LCMS (ESI) m/z: [M+H] calcd for C₂₅H₃₉BrN₄O₇: 587.2; found 587.2.

Step 12. To a solution of (3aS,6S,9S,17aS)-13-bromo-1-(tert-butoxycarbonyl)-6-isopropyl-5-methyl-4,7-dioxo-2,3,3a,4,5,6,7,8,9,10,12,15,17,17a-tetradecahydro-1H-11,15-methanopyrrolo[2,3-c][1]oxa[6,9,12]triazacyclohexadecine-9-carboxylic acid (615 mg, crude), DIPEA (1.35 g, 10.5 mmol) and methyl (S)-hexahydropyridazine-3-carboxylate (301 mg, 2.09 mmol) in DMF (7.0 mL) at 0° C. was added HATU (796 mg, 2.09 mmol). The resulting mixture was stirred for 1 h at room temperature and was then quenched at 0° C. by the addition of H₂O. The aqueous mixture was extracted with EtOAc (3×20 mL), and the combined organic extracts were washed with brine (3×20 mL), dried over Na₂SO₄, filtered, and concentrated under reduced pressure. Purification and diastereomer separation by reverse phase chromatography afforded the following two diastereomers of tert-butyl (3aS,6S,9S,17aS)-13-bromo-6-isopropyl-9-((S)-3-(methoxycarbonyl)hexahydropyridazine-1-carbonyl)-5-methyl-4,7-dioxo-2,3,3a,4,5,6,7,8,9,10,12,15,17,17a-tetradecahydro-1H-11,15-methanopyrrolo[2,3-c][1]oxa[6,9,12]triazacyclohexadecine-1-carboxylate:

Diastereomer 1 (175 mg, 30% yield over 2 steps). LCMS (ESI) m/z: [M+H] calcd for C₃₁H₄₉BrN₆O₈: 713.29; found 713.4.

Diastereomer 2 (66 mg, 11% yield over 2 steps). LCMS (ESI) m/z: [M+H] calcd for C₃₁H₄₉BrN₆O₈: 713.3; found 713.4.

Step 13. To a solution of tert-butyl (3aS,6S,9S,15S,17aS)-13-bromo-6-isopropyl-9-((S)-3-(methoxycarbonyl)hexahydropyridazine-1-carbonyl)-5-methyl-4,7-dioxo-2,3,3a,4,5,6,7,8,9,10,12,15,17,17a-tetradecahydro-1H-11,15-methanopyrrolo[2,3-c][1]oxa[6,9,12]triazacyclohexadecine-1-carboxylate (165 mg, 0.231 mmol, assumed 15S configuration), (S)-(2-(5-(4-cyclopropylpiperazin-1-yl)-2-(1-methoxyethyl)pyridin-3-yl)-1-ethyl-3-(3-hydroxy-2,2-dimethylpropyl)-1H-indol-5-yl)boronic acid (185 mg, 0.347 mmol), and K₃PO₄ (98.2 mg, 0.462 mmol) in toluene (1.8 mL), dioxane (600 μL) and H₂O (600 μL) was added PdCl₂(dtbpf) (15.1 mg, 0.023 mmol). The resulting mixture was stirred for 3 h at 70° C. and was then quenched at 0° C. by the addition of H₂O. The aqueous mixture was extracted with EtOAc (3×10 mL), and the combined organic extracts were dried over Na₂SO₄, filtered, and concentrated under reduced pressure. Purification by normal phase chromatography afforded tert-butyl (3aS,6S,9S,15S,17aS)-13-(2-(5-(4-cyclopropylpiperazin-1-yl)-2-((S)-1-methoxyethyl)pyridin-3-yl)-1-ethyl-3-(3-hydroxy-2,2-dimethylpropyl)-1H-indol-5-yl)-6-isopropyl-9-((S)-3-(methoxycarbonyl)hexahydropyridazine-1-carbonyl)-5-methyl-4,7-dioxo-2,3,3a,4,5,6,7,8,9,10,12,15,17,17a-tetradecahydro-1H-11,15-methanopyrrolo[2,3-c][1]oxa[6,9,12]triazacyclohexadecine-1-carboxylate (163 mg, 56% yield) as a yellow solid. LCMS (ESI) m/z: [M+H] calcd for C₆₁H₉₀N₁₀O₁₀: 1123.7; found 1123.7.

Step 14. To a solution of tert-butyl (3aS,6S,9S,15S,17aS)-13-(2-(5-(4-cyclopropylpiperazin-1-yl)-2-((S)-1-methoxyethyl)pyridin-3-yl)-1-ethyl-3-(3-hydroxy-2,2-dimethylpropyl)-1H-indol-5-yl)-6-isopropyl-9-((S)-3-(methoxycarbonyl)hexahydropyridazine-1-carbonyl)-5-methyl-4,7-dioxo-2,3,3a,4,5,6,7,8,9,10,12,15,17,17a-tetradecahydro-1H-11,15-methanopyrrolo[2,3-c][1]oxa[6,9,12]triazacyclohexadecine-1-carboxylate (160 mg, 0.142 mmol) in THE (1.5 mL) and H₂O (1.5 mL) at 0° C. was added LiOH·H₂O (11.9 mg, 0.284 mmol). The resulting mixture was stirred for 2 h at room temperature and was then acidified to pH=5 with 1M HCl. The aqueous mixture was extracted with DCM (3×10 mL), and the combined organic extracts were dried over Na₂SO₄, filtered, and concentrated under reduced pressure to afford (3S)-1-((3aS,6S,9S,15S,17aS)-1-(tert-butoxycarbonyl)-13-(2-(5-(4-cyclopropylpiperazin-1-yl)-2-((S)-1-methoxyethyl)pyridin-3-yl)-1-ethyl-3-(3-hydroxy-2,2-dimethylpropyl)-1H-indol-5-yl)-6-isopropyl-5-methyl-4,7-dioxo-2,3,3a,4,5,6,7,8,9,10,12,15,17,17a-tetradecahydro-1H-11,15-methanopyrrolo[2,3-c][1]oxa[6,9,12]triazacyclohexadecine-9-carbonyl)hexahydropyridazine-3-carboxylic acid (180 mg, crude) which was taken directly to the next step without further purification. LCMS (ESI) m/z: [M+H] calcd for C₆₀H₈₈N₁₀O₁₀: 1109.7; found 1109.6.

Step 15. To a solution of (3S)-1-((3aS,6S,9S,15S,17aS)-1-(tert-butoxycarbonyl)-13-(2-(5-(4-cyclopropylpiperazin-1-yl)-2-((S)-1-methoxyethyl)pyridin-3-yl)-1-ethyl-3-(3-hydroxy-2,2-dimethylpropyl)-1H-indol-5-yl)-6-isopropyl-5-methyl-4,7-dioxo-2,3,3a,4,5,6,7,8,9,10,12,15,17,17a-tetradecahydro-1H-11,15-methanopyrrolo[2,3-c][1]oxa[6,9,12]triazacyclohexadecine-9-carbonyl)hexahydropyridazine-3-carboxylic acid (175 mg, crude) and DIPEA (612 mg, 4.74 mmol) in DCM (20 mL) at 0° C. were added EDCI (605 mg, 3.16 mmol) and HOBT (213 mg, 1.58 mmol). The resulting mixture was stirred overnight at room temperature and was then acidified to pH=5 with 1M HCl. The aqueous layer was extracted with DCM (3×20 mL), and the combined organic extracts were dried over Na₂SO₄, filtered, and concentrated under reduced pressure. Purification by normal phase prep-TLC afforded tert-butyl (3aS,6S,9S,15S,30S,32aS)-21-(5-(4-cyclopropylpiperazin-1-yl)-2-((S)-1-methoxyethyl)pyridin-3-yl)-22-ethyl-6-isopropyl-5,19,19-trimethyl-4,7,10,16-tetraoxo-2,3,3a,4,5,6,7,8,9,10,13,14,15,16,18,19,20,22,29,30,32,32a-docosahydro-1H,12H,27H-11,15-epimino-23,25-etheno-9,28-methano-26,30-(metheno)dipyrrolo[3,2-b1:3′,4′-i][1,14]dioxa[4,20,23,26]tetraazacyclotriacontine-1-carboxylate (83 mg, 55% yield over 2 steps) as a yellow solid. LCMS (ESI) m/z: [M+H] calcd for C₆₀H₈₆N₁₀O₉: 1091.7; found 1091.7.

Step 16. To a solution of tert-butyl (3aS,6S,9S,15S,30S,32aS)-21-(5-(4-cyclopropylpiperazin-1-yl)-2-((S)-1-methoxyethyl)pyridin-3-yl)-22-ethyl-6-isopropyl-5,19,19-trimethyl-4,7,10,16-tetraoxo-2,3,3a,4,5,6,7,8,9,10,13,14,15,16,18,19,20,22,29,30,32,32a-docosahydro-1H,12H,27H-11,15-epimino-23,25-etheno-9,28-methano-26,30-(metheno)dipyrrolo[3,2-b1:3′,4′-i][1,14]dioxa[4,20,23,26]tetraazacyclotriacontine-1-carboxylate (80 mg, 0.073 mmol) in DCM (2.0 mL) at 0° C. was added TFA (400 μL). The resulting mixture was stirred for 1 h at room temperature and was then basified to pH=8 with sat. aq. NaHCO₃. The aqueous layer was extracted with DCM (3×10 mL), and the combined organic extracts were dried over Na₂SO₄, filtered, and concentrated under reduced pressure to afford (3aS,6S,9S,15S,30S,32aS)-21-(5-(4-cyclopropylpiperazin-1-yl)-2-((S)-1-methoxyethyl)pyridin-3-yl)-22-ethyl-6-isopropyl-5,19,19-trimethyl-3,3a,5,6,8,9,14,15,18,19,20,22,29,30,32,32a-hexadecahydro-1H,12H,27H-11,15-epimino-23,25-etheno-9,28-methano-26,30-(metheno)dipyrrolo[3,2-b1:3′,4′-i][1,14]dioxa[4,20,23,26]tetraazacyclotriacontine-4,7,10,16(2H,13H)-tetraone (61 mg, crude) which was taken directly to the next step without further purification. LCMS (ESI) m/z: [M+H] calcd for C₅₅H₇₈N₁₀O₇: 991.6; found 991.5.

Step 17. To a solution of (3aS,6S,9S,15S,30S,32aS)-21-(5-(4-cyclopropylpiperazin-1-yl)-2-((S)-1-methoxyethyl)pyridin-3-yl)-22-ethyl-6-isopropyl-5,19,19-trimethyl-3,3a,5,6,8,9,14,15,18,19,20,22,29,30,32,32a-hexadecahydro-1H,12H,27H-11,15-epimino-23,25-etheno-9,28-methano-26,30-(metheno)dipyrrolo[3,2-b1:3′,4′-i][1,14]dioxa[4,20,23,26]tetraazacyclotriacontine-4,7,10,16(2H,13H)-tetraone (56.0 mg, crude), DIPEA (73.0 mg, 0.560 mmol) and 2-fluoro-2-methylpropanoic acid (8.9 mg, 0.084 mmol) in DMF (2.0 mL) at 0° C. was added COMU (31.5 mg, 0.073 mmol). The resulting mixture was stirred for 1 h at room temperature then quenched at 0° C. by the addition of H₂O. The aqueous layer was extracted with EtOAc (3×10 mL), and the combined organic extracts were washed with brine (3×20 mL), dried with Na₂SO₄, filtered, and concentrated under reduced pressure. Purification by reverse phase chromatography (52→76% MeOH/H₂O, 10 mM NH₄HCO₃) afforded (3aS,6S,9S,15S,30S,32aS)-21-(5-(4-cyclopropylpiperazin-1-yl)-2-((S)-1-methoxyethyl)pyridin-3-yl)-22-ethyl-1-(2-fluoro-2-methylpropanoyl)-6-isopropyl-5,19,19-trimethyl-3,3a,5,6,8,9,14,15,18,19,20,22,29,30,32,32a-hexadecahydro-1H,12H,27H-11,15-epimino-23,25-etheno-9,28-methano-26,30-(metheno)dipyrrolo[3,2-b1:3′,4′-i][1,14]dioxa[4,20,23,26]tetraazacyclotriacontine-4,7,10,16(2H,13H)-tetraone (15.7 mg, 18% yield over 2 steps). LCMS (ESI) m/z: [M+H] calcd for C₅₉H₈₃FN₁₀O₈: 1079.7; found 1079.6. ¹H NMR (400 MHz, DMSO-d₆) δ 8.44 (s, 1H), 7.60-6.81 (m, 5H), 6.30-6.15 (m, 1H), 5.40-4.91 (m, 1H), 4.84-4.78 (m, 1H), 4.59-4.45 (m, 1H), 4.39-3.55 (m, 11H), 3.25-3.12 (m, 6H), 3.10-3.01 (m, 4H), 2.93-2.80 (m, 6H), 2.71-2.56 (m, 6H), 2.40-2.12 (m, 2H), 2.09-1.96 (m, 1H), 1.89-1.78 (m, 2H), 1.75-1.45 (m, 10H), 1.39-1.17 (m, 7H), 1.00-0.77 (m, 7H), 0.75-0.54 (m, 4H), 0.52-0.10 (m, 7H).

### Example A411. Synthesis of (7S,10S,16S,33aS)-22-(5-(4-cyclopropylpiperazin-1-yl)-2-((S)-1-methoxyethyl)pyridin-3-yl)-23-ethyl-1-(2-fluoro-2-methylpropanoyl)-7-isopropyl-6,20,20-trimethyl-2,3,6,7,9,10,15,16,19,20,21,23,33,33a-tetradecahydro-1H,5H,13H-12,16-epimino-24,26-etheno-10,29-methano-27,31-(metheno)imidazo[2,1-c]pyrrolo[3,4-v][1,18]dioxa[4,6,9,12]tetraazacyclotriacontine-5,8,11,17(14H)-tetraone

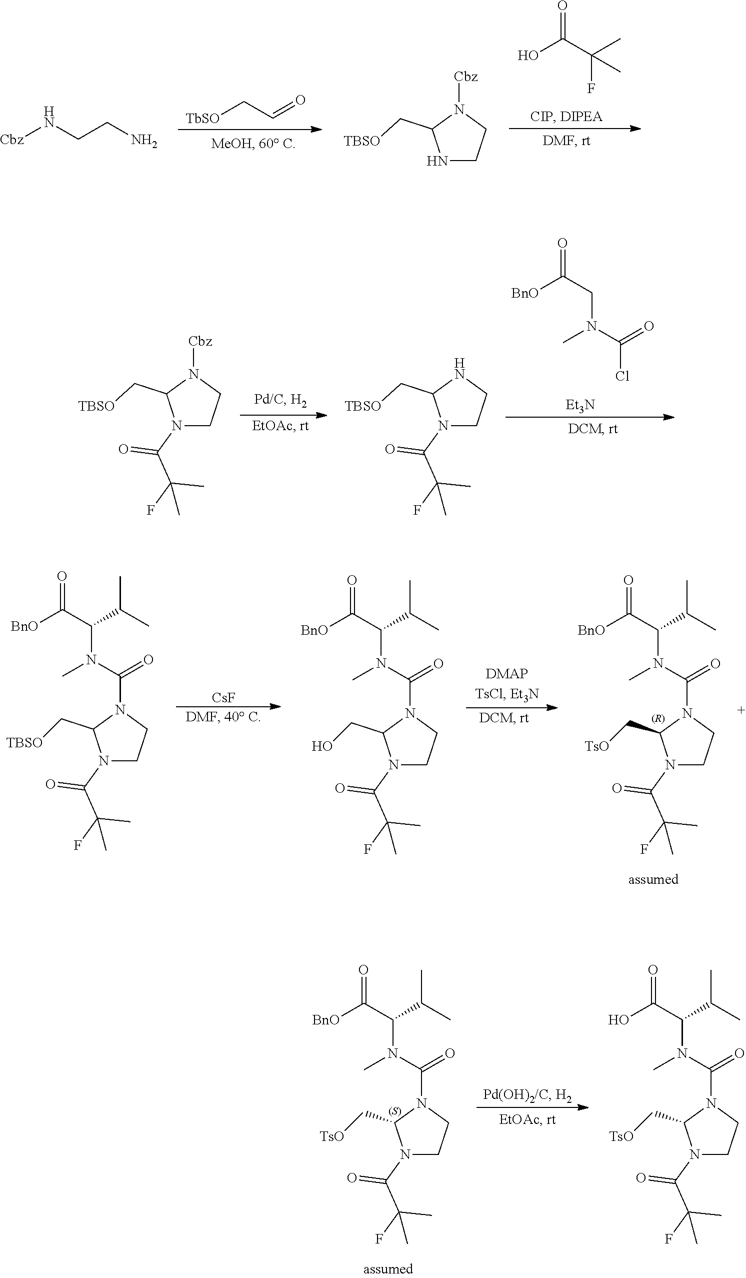

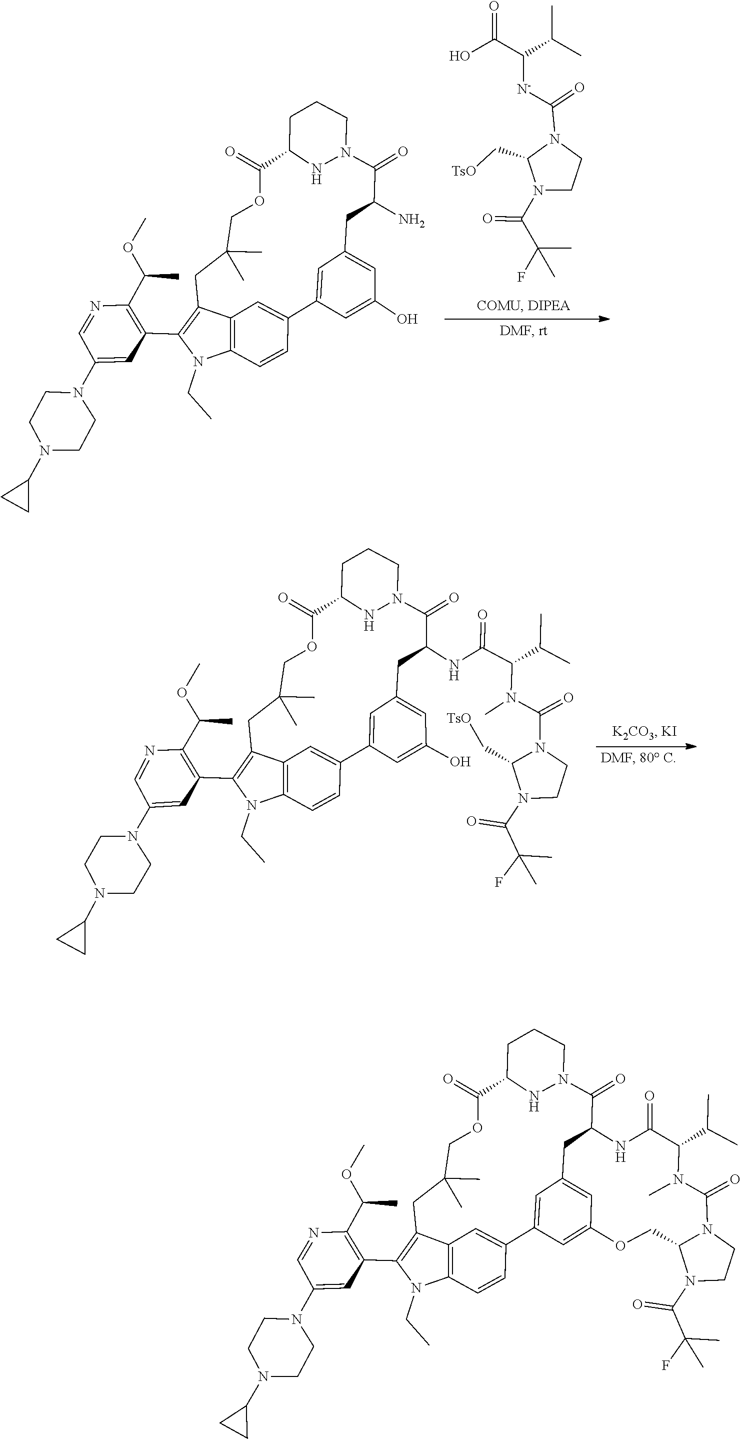

Step 1. To a solution of benzyl (2-aminoethyl)carbamate (3.0 g, 15.4 mmol) in MeOH (240 mL) was added 2-((tert-butyldimethylsilyl)oxy)acetaldehyde (2.7 g, 15.4 mmol). The resulting mixture was stirred overnight at 60° C. and was then concentrated under reduced pressure to afford benzyl 2-(((tert-butyldimethylsilyl)oxy)methyl)imidazolidine-1-carboxylate (5.0 g, crude) which was taken directly to the next reaction without further purification. LCMS (ESI) m/z: [M+H] calcd for C₁₈H₃₀N₂O₃Si: 351.2; found 351.2.

Step 2. To a solution of benzyl 2-(((tert-butyldimethylsilyl)oxy)methyl)imidazolidine-1-carboxylate (5.0 g, crude) and DIPEA (18.4 g, 142 mmol) in DMF (50 mL) stirred at 0° C. was added 2-fluoro-2-methylpropanoic acid (3.18 g, 30.0 mmol) and CIP (7.95 g, 28.5 mmol). The resulting mixture was stirred for 1 h at room temperature and was then neutralized by the addition of sat. aq. NH₄Cl. The aqueous mixture was extracted with EtOAc (3×100 mL), and the combined organic extracts were washed with brine (2×100 mL), dried over Na₂SO₄, filtered, and concentrated under reduced pressure. Purification by reversed phase chromatography afforded benzyl 2-(((tert-butyldimethylsilyl)oxy)methyl)-3-(2-fluoro-2-methylpropanoyl)imidazolidine-1-carboxylate (2.92 g, 54% yield over 2 steps) as an orange oil. LCMS (ESI) m/z: [M+H] calcd for C₂₂H₃₅FN₂O₄Si: 439.3; found 439.3.

Step 3. To a solution of benzyl 2-(((tert-butyldimethylsilyl)oxy)methyl)-3-(2-fluoro-2-methylpropanoyl)imidazolidine-1-carboxylate (2.9 g, 6.61 mmol) in EtOAc (30 mL) was added 10 wt. % Pd/C (3.5 g). The resulting mixture was stirred for 2 h at room temperature under an atmosphere of H₂, filtered, and concentrated under reduced pressure to afford 1-(2-(((tert-butyldimethylsilyl)oxy)methyl)imidazolidin-1-yl)-2-fluoro-2-methylpropan-1-one (1.71 g, crude) which was taken directly to the next reaction without further purification. LCMS (ESI) m/z: [M+H] calcd for C₁₄H₂₉FN₂O₂Si: 305.2; found 305.3.

Step 4. To a solution of 1-(2-(((tert-butyldimethylsilyl)oxy)methyl)imidazolidin-1-yl)-2-fluoro-2-methylpropan-1-one (1.7 g, crude) and Et₃N (1.69 g, 16.7 mmol) in DCM (15 mL) stirred at 0° C. was added a solution of benzyl N-(chlorocarbonyl)-N-methyl-L-valinate (2.06 g, 7.26 mmol) in DCM (2.0 mL). The resulting mixture was stirred for 2 h at room temperature and was then quenched by the addition of H₂O (30 mL). The aqueous phase was extracted with DCM (3×20 mL), and the combined organic extracts were washed with brine (2×30 mL), dried over Na₂SO₄, filtered, and concentrated under reduced pressure. Purification by reversed phase chromatography afforded benzyl N-(2-(((tert-butyldimethylsilyl)oxy)methyl)-3-(2-fluoro-2-methylpropanoyl)imidazolidine-1-carbonyl)-N-methyl-L-valinate (2.88 g, 79% yield over 2 steps) as a dark yellow oil. LCMS (ESI) m/z: [M+H] calcd for C₂₈H₄₆FN₃O₅Si: 552.3; found 552.4.

Step 5. To a solution of benzyl N-(2-(((tert-butyldimethylsilyl)oxy)methyl)-3-(2-fluoro-2-methylpropanoyl)imidazolidine-1-carbonyl)-N-methyl-L-valinate (2.88 g, 5.22 mmol) in DMF (20 mL) was added CsF (3.96 g, 26.1 mmol). The resulting mixture was stirred overnight at 40° C. and was then quenched at 0° C. by the addition of H₂O. The aqueous mixture was extracted with EtOAc (3×20 mL), and the combined organic extracts were washed with brine (2×50 mL), dried over Na₂SO₄, filtered, and concentrated under reduced pressure. Purification by reversed phase chromatography afforded benzyl N-(3-(2-fluoro-2-methylpropanoyl)-2-(hydroxymethyl)imidazolidine-1-carbonyl)-N-methyl-L-valinate (2.23 g, 98% yield) as a yellow oil. LCMS (ESI) m/z: [M+H] calcd for C₂₂H₃₂FN₃O₅: 438.2; found 438.4.

Step 6. To a solution of benzyl N-(3-(2-fluoro-2-methylpropanoyl)-2-(hydroxymethyl)imidazolidine-1-carbonyl)-N-methyl-L-valinate (2.23 g, 5.10 mmol) and Et₃N (1.55 g, 15.3 mmol) in DCM (20 mL) stirred at 0° C. was added DMAP (1.25 g, 10.2 mmol) and a solution of p-toluenesulfonyl chloride (1.17 g, 6.12 mmol) in DCM (3.0 mL). The resulting mixture was stirred for 2 h at room temperature and was then quenched by the addition of H₂O (50 mL). The aqueous phase was extracted with DCM (3×20 mL), and the combined organic extracts were washed with brine (2×50 mL), dried over Na₂SO₄, filtered, and concentrated under reduced pressure. Purification by reversed phase chromatography a mixture of desired products. The diastereomers were separated by normal phase preparative thin layer chromatography to afford benzyl N—((S)-3-(2-fluoro-2-methylpropanoyl)-2-((tosyloxy)methyl)imidazolidine-1-carbonyl)-N-methyl-L-valinate (180 mg, 6% yield, assumed configuration)-LCMS (ESI) m/z: [M+H]calcd for C₂₉H₃₈FN₃O₇S: 592.3; found 592.3—and benzyl N—((R)-3-(2-fluoro-2-methylpropanoyl)-2-((tosyloxy)methyl)imidazolidine-1-carbonyl)-N-methyl-L-valinate (320 mg, 11% yield, assumed configuration). LCMS (ESI) m/z: [M+H] calcd for C₂₉H₃₈FN₃O₇S: 592.3; found 592.3.

Step 7. To a solution of benzyl N—((S)-3-(2-fluoro-2-methylpropanoyl)-2-((tosyloxy)methyl)imidazolidine-1-carbonyl)-N-methyl-L-valinate (180 mg, 0.304 mmol) in EtOAc (5.0 mL) was added 20 wt. % Pd(OH)₂/C (107 mg). The resulting mixture was stirred for 2 h at room temperature under H₂ atmosphere, filtered, and concentrated under reduced pressure to afford N—((S)-3-(2-fluoro-2-methylpropanoyl)-2-((tosyloxy)methyl)imidazolidine-1-carbonyl)-N-methyl-L-valine (150 mg, crude) which was used without further purification. LCMS (ESI) m/z: [M+H] calcd for C₂₂H₃₂FN₃O₇S: 502.2; found 502.2.

Step 8. To a solution of (6³S,4S)-4-amino-1²-(5-(4-cyclopropylpiperazin-1-yl)-2-((S)-1-methoxyethyl)pyridin-3-yl)-1¹-ethyl-25-hydroxy-10,10-dimethyl-6¹,6²,6³,6⁴,6⁵,6⁶-hexahydro-1¹H-8-oxa-1(5,3)-indola-6(1,3)-pyridazina-2(1,3)-benzenacycloundecaphane-5,7-dione (150 mg, 0.196 mmol) and DIPEA (1.02 g, 7.84 mmol) in DMF (5.0 mL) stirred at 0° C. was added COMU (101 mg, 0.235 mmol) and a solution of N—((S)-3-(2-fluoro-2-methylpropanoyl)-2-((tosyloxy)methyl)imidazolidine-1-carbonyl)-N-methyl-L-valine (128 mg, crude) in DMF (500 μL). The resulting mixture was stirred for 30 min at room temperature and was then neutralized by the addition of sat. aq. NH₄Cl. The aqueous mixture was extracted with EtOAc (3×5 mL), and the combined organic extracts were washed with brine (2×30 mL), dried over Na₂SO₄, filtered, and concentrated under reduced pressure. Purification by normal phase preparative thin layer chromatography afforded ((2S)-1-(((2S)-1-(((6³S,4S)-1²-(5-(4-cyclopropylpiperazin-1-yl)-2-((S)-1-methoxyethyl)pyridin-3-yl)-1¹-ethyl-2⁵-hydroxy-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)amino)-3-methyl-1-oxobutan-2-yl)(methyl)carbamoyl)-3-(2-fluoro-2-methylpropanoyl)imidazolidin-2-yl)methyl 4-methylbenzenesulfonate (170 mg, 69% yield) as a yellow solid. LCMS (ESI) m/z: [M+H] calcd for C₆₆H₈₇FN₁₀O₁₁S: 1247.6; found 1247.5.

Step 9. To a solution of ((2S)-1-(((2S)-1-(((6³S,4S)-1²-(5-(4-cyclopropylpiperazin-1-yl)-2-((S)-1-methoxyethyl)pyridin-3-yl)-1¹-ethyl-2⁵-hydroxy-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)amino)-3-methyl-1-oxobutan-2-yl)(methyl)carbamoyl)-3-(2-fluoro-2-methylpropanoyl)imidazolidin-2-yl)methyl 4-methylbenzenesulfonate (85 mg, 0.068 mmol) in DMF (9.0 mL) was added K₂CO₃ (94.2 mg, 0.680 mmol) and KI (11.3 mg, 0.068 mmol). The resulting mixture was stirred for 1 h at 80° C. and was then quenched at room temperature by the addition of H₂O (50 mL). The aqueous mixture was extracted with EtOAc (3×10 mL), and the combined organic extracts were washed with brine (3×50 mL), dried over Na₂SO₄, filtered, and concentrated under reduced pressure. Purification by normal phase preparative thin layer chromatography followed by reversed phase chromatography afforded (7S,10S,16S,33aS)-22-(5-(4-cyclopropylpiperazin-1-yl)-2-((S)-1-methoxyethyl)pyridin-3-yl)-23-ethyl-1-(2-fluoro-2-methylpropanoyl)-7-isopropyl-6,20,20-trimethyl-2,3,6,7,9,10,15,16,19,20,21,23,33,33a-tetradecahydro-1H,5H,13H-12,16-epimino-24,26-etheno-10,29-methano-27,31-(metheno)imidazo[2,1-c]pyrrolo[3,4-v][1,18]dioxa[4,6,9,12]tetraazacyclotriacontine-5,8,11,17(14F)-tetraone (12 mg, 17% yield) as a white solid. LCMS (ESI) m/z: [M+H] calcd for C₅₉H₇₉FN₁₀O₈: 1075.6; found 1075.6. ¹H NMR (400 MHz, DMSO-d₆) δ 9.26 (s, 1H), 8.44 (d, J=2.8 Hz, 1H), 7.92 (s, 1H), 7.57 (d, J=8.7 Hz, 1H), 7.53 (d, J=8.7 Hz, 1H), 7.23 (d, J=3.0 Hz, 2H), 6.95 (s, 1H), 6.49 (s, 1H), 5.27 (s, 1H), 5.16 (d, J=12.0 Hz, 1H), 5.07 (d, J=9.8 Hz, 1H), 4.25-4.02 (m, 4H), 3.95 (t, J=7.6 Hz, 1H), 3.77-3.56 (m, 4H), 3.50 (d, J=8.7 Hz, 1H), 3.22 (d, J=10.6 Hz, 1H), 3.09 (s, 3H), 3.04 (d, J=2.7 Hz, 1H), 2.88-2.70 (m, 4H), 2.68 (t, J=4.9 Hz, 6H), 2.20 (s, 3H), 2.09 (s, 1H), 1.99 (d, J=11.6 Hz, 1H), 1.80 (s, 1H), 1.66 (dt, J=6.5, 3.0 Hz, 1H), 1.49 (d, J=2.9 Hz, 4H), 1.43-1.39 (m, 7H), 1.24 (s, 1H), 1.10-1.03 (m, 11H), 0.77 (s, 3H), 0.67 (s, 1H), 0.53 (s, 3H), 0.43 (dd, J=6.7, 4.4 Hz, 2H), 0.34 (t, J=3.0 Hz, 3H).

### Example A415. Synthesis of (2⁵S,2⁸S,2^{10a}S,2^{13a}R,4³S,Z)-12-(5-(4-cyclopropylpiperazin-1-yl)-2-((S)-1-methoxyethyl)pyridin-3-yl)-1¹-ethyl-2¹³-(2-fluoro-2-methylpropanoyl)-2⁸-isopropyl-2⁹,8,8-trimethyl-2⁴,2⁵,2⁶,2⁷,2⁸,2⁹,2¹⁰,2^{10a},2¹¹,2¹²,2¹³,2^{13a},4¹,4²,4³,4⁴,4⁵,4⁶-icosahydro-1¹H-6-oxa-2(2,5)-pyrrolo[2,3-k][1,2,4]triazolo[5,1-g][1,4,8]triazacyclotridecina-1(5,3)-indola-4(1,3)-pyridazinacyclononaphane-2⁷,2¹⁰,3,5-tetraone

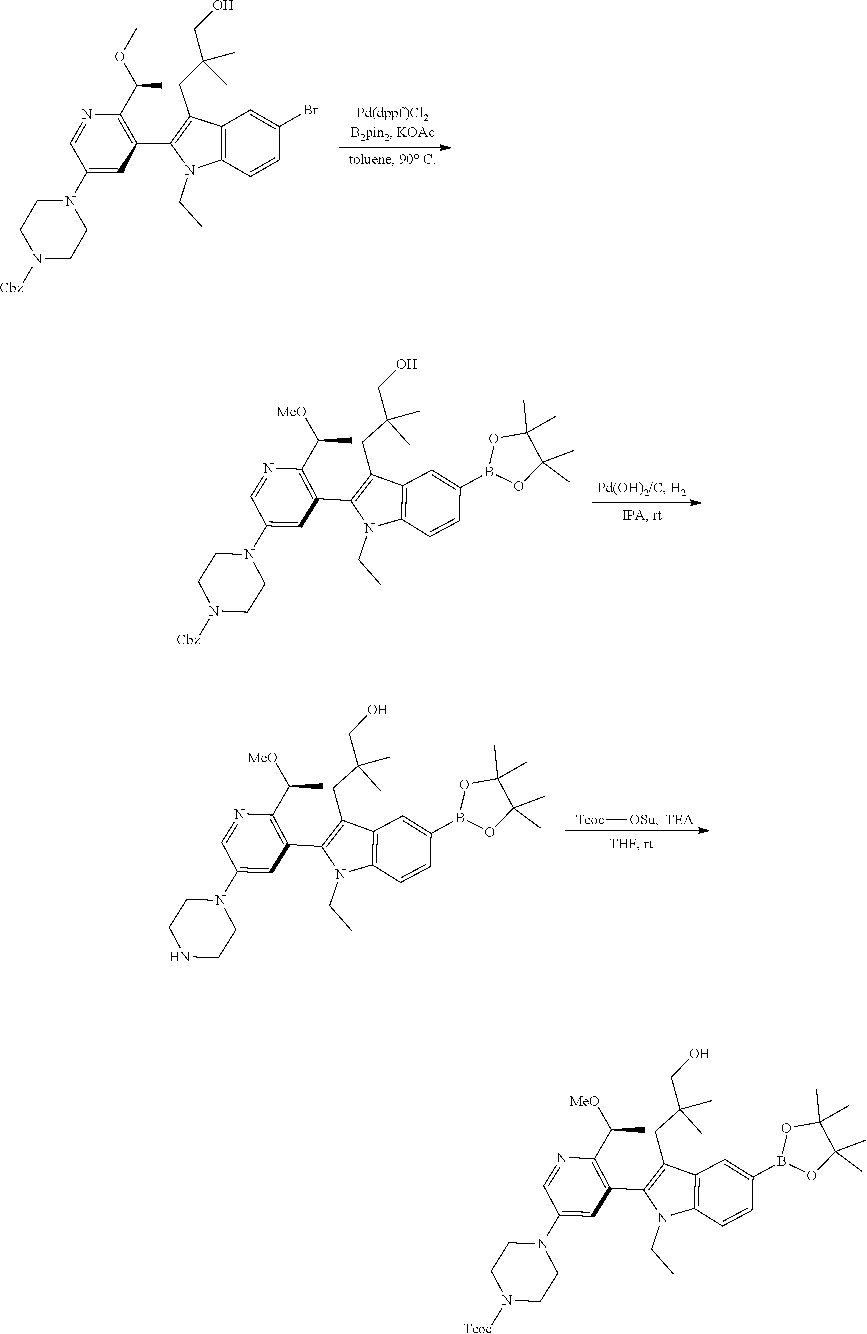

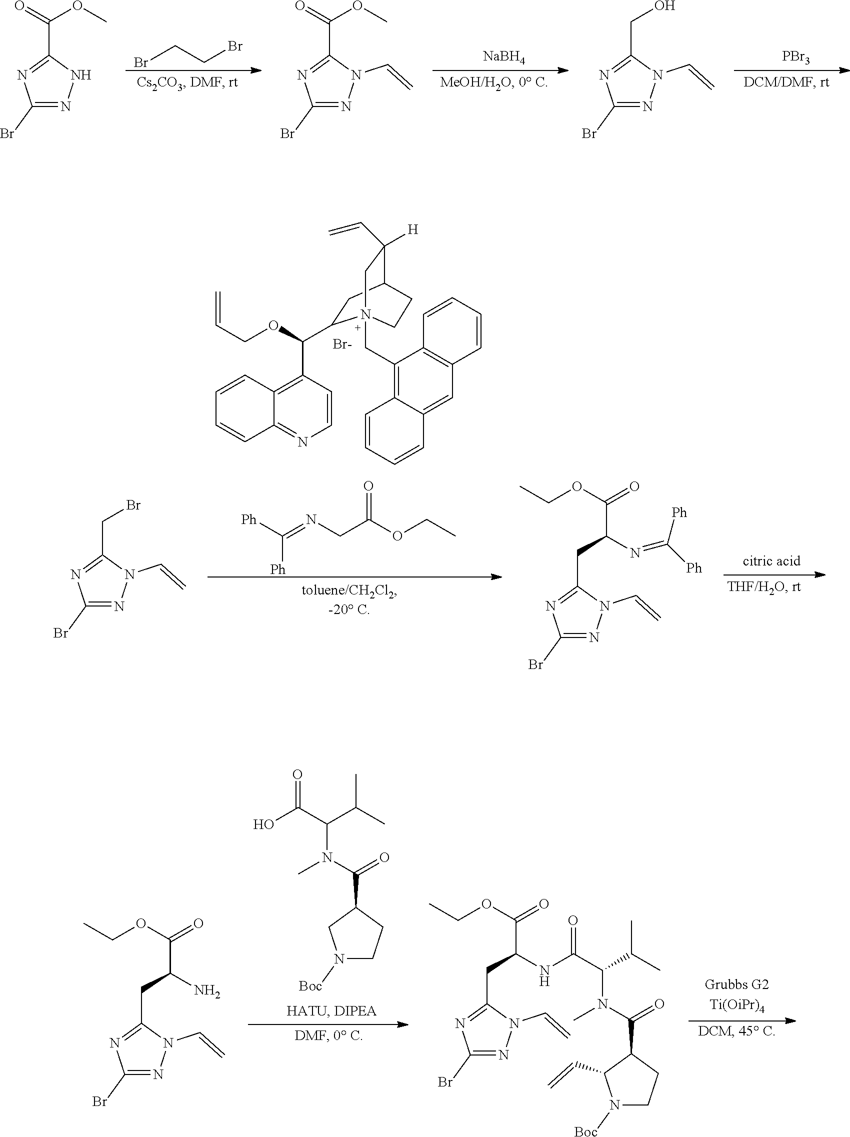

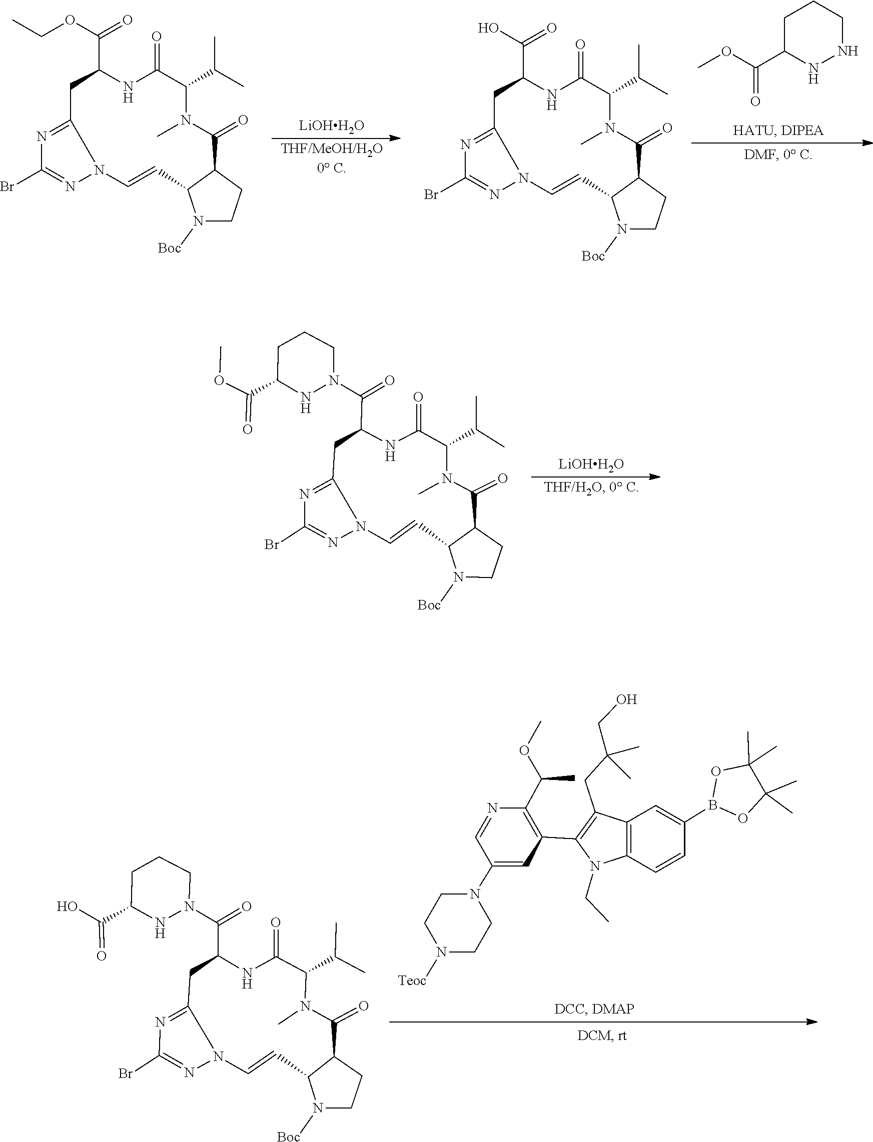

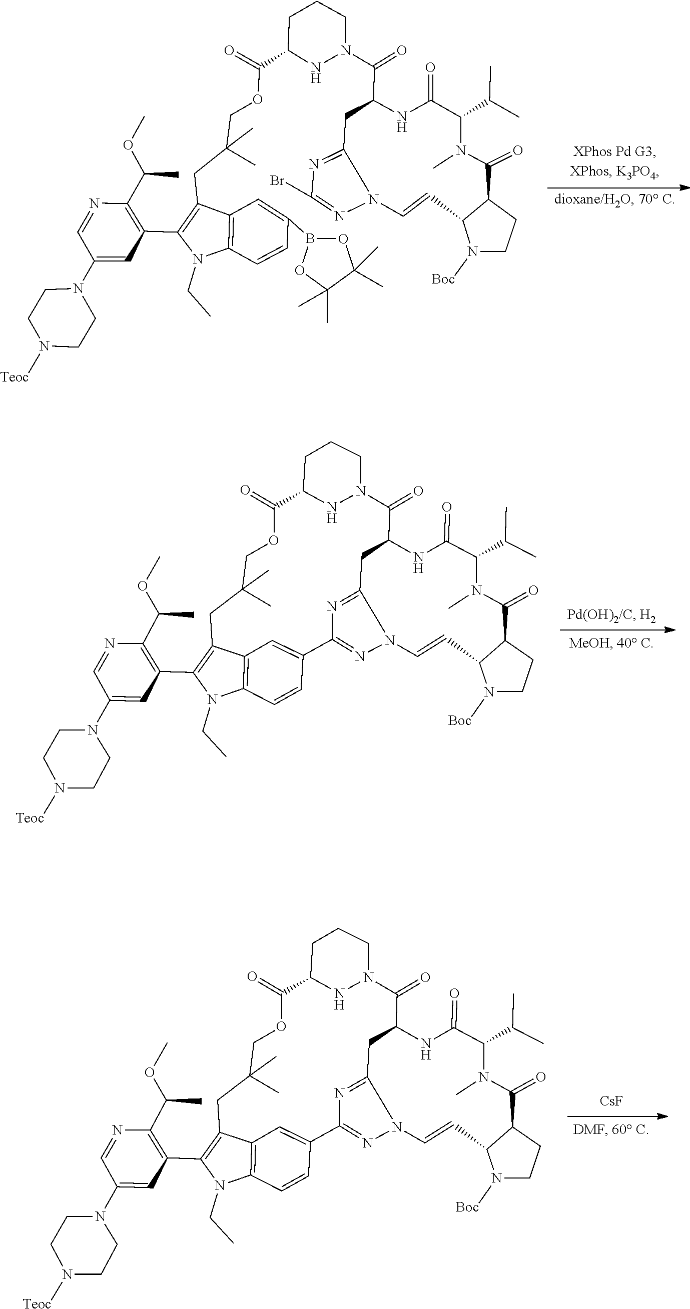

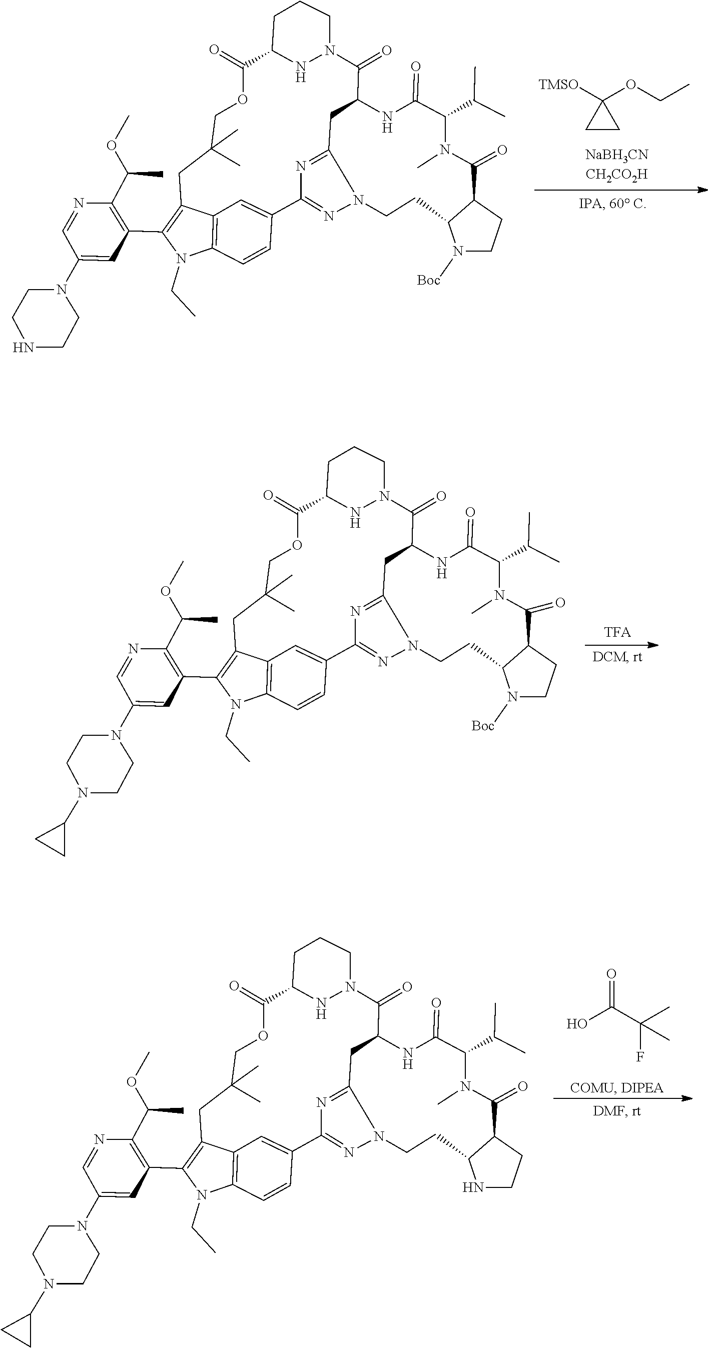

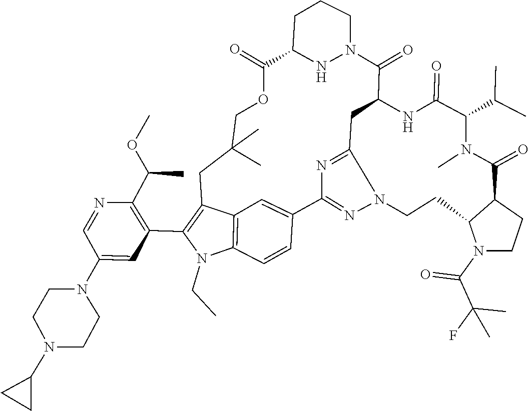

Step 1. To a solution of benzyl (S)-4-(5-(5-bromo-1-ethyl-3-(3-hydroxy-2,2-dimethylpropyl)-1H-indol-2-yl)-6-(1-methoxyethyl)pyridin-3-yl)piperazine-1-carboxylate (8.34 g, 12.6 mmol) and B2pin₂ (9.6 g, 38 mmol) in toluene (85 mL) at room temperature were added KOAc (2.47 g, 25.2 mmol) and Pd(dppfCl₂ (2.06 g, 2.52 mmol). The resulting mixture was stirred for 2 h at 90° C. and was then quenched at 0° C. by the addition of H₂O (50 mL). The resulting mixture was extracted with EtOAc (3×30 mL) and the combined organic extracts were dried over Na₂SO₄, filtered, and concentrated under reduced pressure. Purification by normal phase chromatography afforded benzyl (S)-4-(5-(1-ethyl-3-(3-hydroxy-2,2-dimethylpropyl)-5-(4,4,5,5-tetramethyl-1,3,2-dioxaborolan-2-yl)-1H-indol-2-yl)-6-(1-methoxyethyl)pyridin-3-yl)piperazine-1-carboxylate (6.18 g, 69% yield) as a light brown solid. LCMS (ESI) m/z: [M+H] calcd for C₄₁H₅₅BrN₄O₆: 711.4; found 711.3.

Step 2. To a solution of benzyl (S)-4-(5-(1-ethyl-3-(3-hydroxy-2,2-dimethylpropyl)-5-(4,4,5,5-tetramethyl-1,3,2-dioxaborolan-2-yl)-1H-indol-2-yl)-6-(1-methoxyethyl)pyridin-3-yl)piperazine-1-carboxylate (6.18 g, 8.69 mmol) in IPA (60 mL) at room temperature was added Pd(OH)₂/C (4.0 g). The resulting mixture was stirred for 6 h under an atmosphere of H₂, filtered, and the filter cake washed with MeOH (10×20 ml). The filtrate was concentrated under reduced pressure to afford (S)-3-(1-ethyl-2-(2-(1-methoxyethyl)-5-(piperazin-1-yl)pyridin-3-yl)-5-(4,4,5,5-tetramethyl-1,3,2-dioxaborolan-2-yl)-1H-indol-3-yl)-2,2-dimethylpropan-1-ol (4.91 g, crude) which taken directly to the next reaction without further purification. LCMS (ESI) m/z: [M+H] calcd for C₃₃H₄₉BN₄O₄: 577.4; found 577.4.

Step 3. To a solution of (S)-3-(1-ethyl-2-(2-(1-methoxyethyl)-5-(piperazin-1-yl)pyridin-3-yl)-5-(4,4,5,5-tetramethyl-1,3,2-dioxaborolan-2-yl)-1H-indol-3-yl)-2,2-dimethylpropan-1-ol (4.91 g, crude) and Et₃N (2.59 g, 25.5 mmol) in THE (50 mL) at 0° C. was added Teoc-OSu (4.42 g, 17.0 mmol). The resulting mixture was stirred for 12 h at room temperature and was then quenched at 0° C. by the addition of H₂O (50 mL). The aqueous mixture was extracted with EtOAc (3×30 mL) and the combined organic extracts were dried with Na₂SO₄, filtered, and concentrated under reduced pressure. Purification by normal phase chromatography afforded 2-(trimethylsilyl)ethyl (S)-4-(5-(1-ethyl-3-(3-hydroxy-2,2-dimethylpropyl)-5-(4,4,5,5-tetramethyl-1,3,2-dioxaborolan-2-yl)-1H-indol-2-yl)-6-(1-methoxyethyl)pyridin-3-yl)piperazine-1-carboxylate (5.74 g, 87% yield over 2 steps) as a brown solid. LCMS (ESI) m/z: [M+H] calcd for C₃₉H₆₁BN₄O₆Si: 721.5; found 721.4.

Step 4. To a solution of methyl 3-bromo-1H-1,2,4-triazole-5-carboxylate carboxylate (10.0 g, 48.5 mmol) and Cs₂CO₃ (7.91 g, 242 mmol) in DMF (100 mL) at 0° C. was added 1,2-dibromoethane (4.57 g, 242 mmol). The resulting mixture was stirred for 3 h at room temperature and was then quenched at 0° C. by the addition of H₂O (50 mL). The aqueous mixture was extracted with EtOAc (3×50 mL) and the combined organic extracts were washed with brine (3×300 mL), dried over Na₂SO₄, filtered, and concentrated under reduced pressure. Purification by normal phase chromatography afforded methyl 3-bromo-1-vinyl-1H-1,2,4-triazole-5-carboxylate (1.60 g, 14% yield) as a white solid. LCMS (ESI) m/z: [M+H] calcd for C₆H₆BrN₃O₂: 232.0; found 231.9.

Step 5. To a solution of methyl 3-bromo-1-vinyl-1H-1,2,4-triazole-5-carboxylate (3.70 g, 15.9 mmol) in MeOH (30 mL) and H₂O (10 mL) at 0° C. was added NaBH₄ (2.41 g, 63.8 mmol). The resulting mixture was stirred for 3 h at 0° C., quenched by the addition of H₂O (20 mL), and concentrated under reduced pressure. The concentrate was extracted with EtOAc (3×15 mL), and the combined organic extracts were dried over Na₂SO₄, filtered, and concentrated under reduced pressure. Purification by normal phase chromatography afforded (3-bromo-1-vinyl-1H-1,2,4-triazol-5-yl)methanol (2.85 g, 88% yield) as a light yellow oil. LCMS (ESI) m/z: [M+H] calcd for C₅H₆BrN₃O: 204.0; found 204.1.

Step 6. To a solution of (3-bromo-1-vinyl-1H-1,2,4-triazol-5-yl)methanol (603 mg, 2.96 mmol) in DCM (10 mL) and DMF (2.0 mL) at 0° C. was added PBr₃ (1.2 g, 4.4 mmol). The resulting mixture was stirred for 10 h at 25° C. and was then quenched at 0° C. by the addition of H₂O. The aqueous mixture was extracted with DCM (3×15 mL) and the combined organic extracts were dried over Na₂SO₄, filtered, and concentrated under reduced pressure. Purification by normal phase chromatography afforded 3-bromo-5-(bromomethyl)-1-vinyl-1H-1,2,4-triazole (739 mg, 94% yield) as a white solid. LCMS (ESI) m/z: [M+H] calcd for C₅H₅Br₂N₃: 265.9; found 266.0.

Step 7. To a solution of ethyl 2-((diphenylmethylene)amino)acetate (3.30 g, 12.4 mmol) in toluene (21 mL) and DCM (9.0 mL) at −20° C. were added O-allyl-N-(9-anthracenylmethyl)cinchonidinium bromide (749 mg, 1.24 mmol) and 9M aq. KOH (30 mL) followed by 3-bromo-5-(bromomethyl)-1-vinyl-1H-1,2,4-triazole (3.3 g, 12.4 mmol). The resulting mixture was stirred overnight at −20° C. and was then quenched at −30° C. by the addition of H₂O (30 mL). The aqueous mixture was extracted with EtOAc (3×20 mL) and the combined organic extracts were dried over Na₂SO₄, filtered, and concentrated under reduced pressure to afford ethyl (S)-3-(3-bromo-1-vinyl-1H-1,2,4-triazol-5-yl)-2-((diphenylmethylene)amino)propanoate (6.0 g, crude) which was taken directly to the next reaction without further purification. LCMS (ESI) m/z: [M+H] calcd for C₂₂H₂₁BrN₄O₂: 453.1; found 453.1.

Step 8. To a solution of ethyl (S)-3-(3-bromo-1-vinyl-1H-1,2,4-triazol-5-yl)-2-((diphenylmethylene)amino)propanoate (6.0 g, crude) in THE (30 mL) and H₂O (30 mL) at room temperature was added citric acid (12.7 g, 66.8 mmol). The resulting mixture was stirred at room temperature for 5 h and was then quenched at 0° C. by the addition of H₂O (20 mL). The aqueous mixture was extracted with MTBE (3×20 mL) and the combined MTBE extracts were discarded. The aqueous phase was adjusted to pH=8 with 1M aq. NaHCO₃ and extracted with DCM (3×20 mL). The combined organic extracts were dried over Na₂SO₄, filtered, and concentrated under reduced pressure. Purification by normal phase chromatography afforded ethyl (S)-2-amino-3-(3-bromo-1-vinyl-1H-1,2,4-triazol-5-yl)propanoate (2.43 g, 68% yield over 2 steps) as a colorless oil. LCMS (ESI) m/z: [M+H] calcd for C₁₉H₁₃BrN₄O₂: 289.0; found 289.0.

Step 9. To a solution of ethyl (S)-2-amino-3-(3-bromo-1-vinyl-1H-1,2,4-triazol-5-yl)propanoate (1.60 g, 5.53 mmol) and DIPEA (28.6 g, 221 mmol) in DMF (20 mL) at stirred at 0° C. were added N-((2R,3S)-1-(tert-butoxycarbonyl)-2-vinylpyrrolidine-3-carbonyl)-N-methyl-L-valine (2.55 g, crude) and HATU (2.73 g, 7.19 mmol). The resulting mixture was stirred for 2 h at 0° C. and was then quenched at 0° C. by the addition of H₂O (10 mL). The aqueous mixture was extracted with EtOAc (3×10 mL) and combined organic extracts were washed with brine (3×30 mL), dried over Na₂SO₄, filtered, and concentrated under reduced pressure. Purification by normal phase chromatography afforded tert-butyl (2R,3S)-3-(((S)-1-(((S)-3-(3-bromo-1-vinyl-1H-1,2,4-triazol-5-yl)-1-ethoxy-1-oxopropan-2-yl)amino)-3-methyl-1-oxobutan-2-yl)(methyl)carbamoyl)-2-vinylpyrrolidine-1-carboxylate (2.52 g, 73% yield) as a light brown oil. LCMS (ESI) m/z: [M+H] calcd for C₂₇H₄₁BrN₆O₆: 625.2; found 625.2.

Step 10. To a solution of tert-butyl (2R,3S)-3-(((S)-1-(((S)-3-(3-bromo-1-vinyl-1H-1,2,4-triazol-5-yl)-1-ethoxy-1-oxopropan-2-yl)amino)-3-methyl-1-oxobutan-2-yl)(methyl)carbamoyl)-2-vinylpyrrolidine-1-carboxylate (3.34 g, 5.34 mmol) and Ti(OiPr)₄ (911 mg, 3.20 mmol) in DCM (350 mL) at room temperature was added Grubbs G2 catalyst (2.72 g, 3.20 mmol). The resulting mixture was stirred for 12 h at 45° C. and was then quenched at 0° C. by the addition of H₂O (30 mL). The aqueous mixture was extracted with DCM (3×30 mL) and the combined organic extracts were dried over Na₂SO₄, filtered, and concentrated under reduced pressure. Purification by reversed phase chromatography afforded 13-(tert-butyl) 5-ethyl (5S,8S,10aS,13aR,E)-2-bromo-8-isopropyl-9-methyl-7,10-dioxo-5,6,7,8,9,10,10a,11,12,13a-decahydropyrrolo[2,3-k][1,2,4]triazolo[5,1-g][1,4,8]triazacyclotridecine-5,13(4H)-dicarboxylate (864 mg, 27% yield) as a light brown oil. LCMS (ESI) m/z: [M+H] calcd for C₂₅H₃₇BrN₆O₆: 597.2; found 597.2.

Step 11. To a solution of 13-(tert-butyl) 5-ethyl (5S,8S,10aS,13aR,E)-2-bromo-8-isopropyl-9-methyl-7,10-dioxo-5,6,7,8,9,10,10a,11,12,13a-decahydropyrrolo[2,3-k][1,2,4]triazolo[5,1-g][1,4,8]triazacyclotridecine-5,13(4H)-dicarboxylate (864 mg, 1.45 mmol) in THE (6.0 mL), MeOH (2.0 mL), and H₂O (2.0 mL) at 0° C. was added LiOH·H₂O (121 mg, 2.89 mmol). The resulting mixture was stirred for 3 h, concentrated under reduced pressure, and the concentrate acidified to pH=5 with 1M aq. HCl. The aqueous mixture was extracted with DCM (3×25 mL), and the combined organic extracts were dried over Na₂SO₄, filtered, and concentrated under reduced pressure to afford (5S,8S,10aS,13aR,E)-2-bromo-13-(tert-butoxycarbonyl)-8-isopropyl-9-methyl-7,10-dioxo-4,5,6,7,8,9,10,10a,11,12,13,13a-dodecahydropyrrolo[2,3-k][1,2,4]triazolo[5,1-g][1,4,8]triazacyclotridecine-5-carboxylic acid (775 mg, crude) which was taken directly to the next reaction without further purification. LCMS (ESI) m/z: [M+H] calcd for C₂₃H₃₃BrN₆O₆: 569.2; found 569.1.

Step 12. To a solution of methyl (S)-hexahydropyridazine-3-carboxylate (291 mg, 2.01 mmol) and DIPEA (5.20 g, 40.3 mmol) in DMF (10 mL) stirred at 0° C. were added (5S,8S,10aS,13aR,E)-2-bromo-13-(tert-butoxycarbonyl)-8-isopropy-9-methy-7,10-dioxo-4,5,6,7,8,9,10,10a,11,12,13,13a-dodecahydropyrrolo[2,3-k][1,2,4]triazolo[5,1-g][1,4,8]triazacyclotridecine-5-carboxylic acid (765 mg, crude) and HATU (1.02 g, 2.69 mmol). The resulting mixture was stirred for 2 h and was then quenched at 0° C. by the addition of H₂O (10 mL). The aqueous mixture was extracted with EtOAc (3×10 mL) and the combined organic extracts were washed with brine (3×30 mL), dried over Na₂SO₄, filtered, and concentrated under reduced pressure. Purification by normal phase prep-TLC afforded tert-butyl (5S,8S,10aS,13aR,E)-2-bromo-8-isopropyl-5-((S)-3-(methoxycarbonyl)hexahydropyridazine-1-carbonyl)-9-methyl-7,10-dioxo-5,6,7,8,9,10,10a,11,12,13a-decahydropyrrolo[2,3-k][1,2,4]triazolo[5,1-g][1,4,8]triazacyclotridecine-13(4H)-carboxylate (549 mg, 54% yield over 2 steps) as a light brown oil. LCMS (ESI) m/z: [M+H] calcd for C₂₉H₄₃BrN₈O₇: 695.3; found 695.3.

Step 13. To a solution of tert-butyl (5S,8S,10aS,13aR,E)-2-bromo-8-isopropyl-5-((S)-3-(methoxycarbonyl)hexahydropyridazine-1-carbonyl)-9-methyl-7,10-dioxo-5,6,7,8,9,10,10a,11,12,13a-decahydropyrrolo[2,3-k][1,2,4]triazolo[5,1-g][1,4,8]triazacyclotridecine-13(4H)-carboxylate (549 mg, 0.789 mmol) in THE (6 mL) and H₂O (2 mL) stirred at 0° C. was added LiOH·H₂O (66.2 mg, 1.58 mmol). The resulting mixture was stirred for 3 h at 0° C., concentrated under reduced pressure, and the concentrate acidified to pH=5 with 1 M aq. HCl. The aqueous mixture was extracted with DCM (3×25 mL) and the combined organic extracts were dried over Na₂SO₄, filtered, and concentrated under reduced pressure to afford (S)-1-((5S,8S,10aS,13aR,E)-2-bromo-13-(tert-butoxycarbonyl)-8-isopropyl-9-methyl-7,10-dioxo-4,5,6,7,8,9,10,10a,11,12,13,13a-dodecahydropyrrolo[2,3-k][1,2,4]triazolo[5,1-g][1,4,8]triazacyclotridecine-5-carbonyl)hexahydropyridazine-3-carboxylic acid (498 mg, crude) which was taken directly to the next reaction without further purification. LCMS (ESI) m/z: [M+H] calcd for C₂₈H₄₁BrN₈O₇: 681.2; found 681.2.

Step 14. To a solution of (S)-1-((5S,8S,10aS,13aR,E)-2-bromo-13-(tert-butoxycarbonyl)-8-isopropyl-9-methyl-7,10-dioxo-4,5,6,7,8,9,10,10a,11,12,13,13a-dodecahydropyrrolo[2,3-k][1,2,4]triazolo[5,1-g][1,4,8]triazacyclotridecine-5-carbonyl)hexahydropyridazine-3-carboxylic acid (498 mg, crude) and DMAP (8.9 mg, 0.073 mmol) in DCM (5.0 mL) stirred at 0° C. were added 2-(trimethylsilyl)ethyl (S)-4-(5-(1-ethyl-3-(3-hydroxy-2,2-dimethylpropyl)-5-(4,4,5,5-tetramethyl-1,3,2-dioxaborolan-2-yl)-1H-indol-2-yl)-6-(1-methoxyethyl)pyridin-3-yl)piperazine-1-carboxylate (790 mg, 1.10 mmol) and DCC (452 mg, 2.19 mmol). The resulting mixture was stirred for 3 h at room temperature and was then quenched at 0° C. by the addition of H₂O (10 mL). The aqueous mixture was extracted with DCM (3×15 mL) and the combined organic extracts were dried over Na₂SO₄, filtered, and concentrated under reduced pressure. Purification by normal phase chromatography afforded tert-butyl (5S,8S,10aS,13aR,E)-2-bromo-5-((S)-3-((3-(1-ethyl-2-(2-((S)-1-methoxyethyl)-5-(4-((2-(trimethylsilyl)ethoxy)carbonyl)piperazin-1-yl)pyridin-3-yl)-5-(4,4,5,5-tetramethyl-1,3,2-dioxaborolan-2-yl)-1H-indol-3-yl)-2,2-dimethylpropoxy)carbonyl)hexahydropyridazine-1-carbonyl)-8-isopropyl-9-methyl-7,10-dioxo-5,6,7,8,9,10,10a,11,12,13a-decahydropyrrolo[2,3-k][1,2,4]triazolo[5,1-g][1,4,8]triazacyclotridecine-13(4H)-carboxylate (348 mg, 31% yield over 2 steps) as a light brown solid. LCMS (ESI) m/z: [M+H] calcd for C₆₇H₁₀₀BBrN₁₂O₁₂Si: 1385.4; found 1385.6.

Step 15. To a solution of tert-butyl (5S,8S,10aS,13aR,E)-2-bromo-5-((S)-3-((3-(1-ethyl-2-(2-((S)-1-methoxyethyl)-5-(4-((2-(trimethylsilyl)ethoxy)carbonyl)piperazin-1-yl)pyridin-3-yl)-5-(4,4,5,5-tetramethyl-1,3,2-dioxaborolan-2-yl)-1H-indol-3-yl)-2,2-dimethylpropoxy)carbonyl)hexahydropyridazine-1-carbonyl)-8-isopropyl-9-methyl-7,10-dioxo-5,6,7,8,9,10,10a,11,12,13a-decahydropyrrolo[2,3-k][1,2,4]triazolo[5,1-g][1,4,8]triazacyclotridecine-13(4H)-carboxylate (330 mg, 0.238 mmol), XPhos (34.1 mg, 0.071 mmol), and XPhos Pd G3 (121 mg, 0.143 mmol) in dioxane (40 mL) and H₂O (8.0 mL) stirred at room temperature was added K₃PO₄ (75.9 mg, 0.357 mmol). The resulting mixture was stirred for 3 h at 70° C. and was then quenched at 0° C. by the addition of H₂O (10 mL). The aqueous mixture was extracted with EtOAc (3×15 mL) and the combined organic extracts were dried over Na₂SO₄, filtered, and concentrated under reduced pressure. Purification by normal phase prep-TLC afforded tert-butyl (2³Z,2⁵S,2⁸S,2^{10a}S,2^{13a}R,2¹⁴E,4³S)-1¹-ethyl-2⁸-isopropyl-1²-(2-((S)-1-methoxyethyl)-5-(4-((2-(trimethylsilyl)ethoxy)carbonyl)piperazin-1-yl)pyridin-3-yl)-2⁹,8,8-trimethyl-2⁷,2¹⁰,3,5-tetraoxo-2⁴,2⁵,2⁶,2⁷,2⁸,2⁹,2¹⁰,2^{10a},2¹¹,2¹²,2¹³,2^{13a},4¹,4²,4³,4⁴,4⁵,4⁶-octadecahydro-1 ¹H-6-oxa- 2(2,5)-pyrrolo[2,3-k][1,2,4]triazolo[5,1-g][1,4,8]triazacyclotridecina-1 (5,3)-indola-4(1,3)-pyridazinacyclononaphane-2¹³-carboxylate (115 mg, 41% yield) as a light brown solid. LCMS (ESI) m/z: [M+H] calcd for C₆₁H₈₈N₁₂O₁₀Si: 1177.7; found 1177.8.

Step 16. To a solution of tert-butyl (2³Z,2⁵S,2⁸S,2^{10a}S,2^{13a}R,2¹⁴E,4³S)-1¹-ethyl-2⁸-isopropyl-1²-(2-((S)-1-methoxyethyl)-5-(4-((2-(trimethylsilyl)ethoxy)carbonyl)piperazin-1-yl)pyridin-3-yl)-2⁹,8,8-trimethyl-2⁷,2¹⁰,3,5-tetraoxo-2⁴,2⁵,2⁶,2⁷,2⁸,2⁹,2¹⁰,2^{10a},2¹¹,2¹²,2¹³,2^{13a},4¹,4²,4³,4⁴,4⁵,4⁶- octadecahydro-1¹H-6-oxa-2(2,5)-pyrrolo[2,3-k][1,2,4]triazolo[5,1-g][1,4,8]triazacyclotridecina-1(5,3)-indola-4(1,3)-pyridazinacyclononaphane-2¹³-carboxylate (125 mg, 0.107 mmol) in MeOH (2.0 mL) stirred at room temperature was added Pd(OH)₂/C (300 mg). The resulting mixture was stirred for 12 h at 40° C. under an atmosphere of H₂ (6 MPa), filtered, and the filter cake washed with MeOH (3×10 mL). The filtrate was concentrated under reduced pressure to afford tert-butyl (2⁵S,2⁸S,2^{10a}S,2^{13a}R,4³S,Z)-1¹-ethyl-28-isopropyl-1²-(2-((S)-1-methoxyethyl)-5-(4-((2-(trimethylsilyl)ethoxy)carbonyl)piperazin-1-yl)pyridin-3-yl)-2⁹,8,8-trimethyl-2⁷,2¹⁰,3,5-tetraoxo-2⁴,2⁵,2⁶,2⁷,2⁸,2⁹,2¹⁰,2^{10a},2¹¹,2¹²,2¹³,2^{13a},4¹,4²,4³,4⁴,4⁵,4⁶-icosahydro-1¹H-6-oxa-2(2,5)-pyrrolo[2,3-k][1,2,4]triazolo[5,1-g][1,4,8]triazacyclotridecina-1(5,3)-indola-4(1,3)-pyridazinacyclononaphane-2¹³-carboxylate (101 mg, crude), which was taken to the next reaction without further purification. LCMS (ESI) m/z: [M+H] calcd for C₆₁H₉₀N₁₂O₁₀Si: 1179.7; found 1179.7.

Step 17. To a solution of tert-butyl (2⁵S,2⁸S,2^{10a}S,2^{13a}R,4³S,Z)-11-ethyl-28-isopropyl-12-(2-((S)-1-methoxyethyl)-5-(4-((2-(trimethylsilyl)ethoxy)carbonyl)piperazin-1-yl)pyridin-3-yl)-2⁹,8,8-trimethyl-2⁷,2¹⁰,3,5-tetraoxo-2⁴,2⁵,2⁶,2⁷,2⁸,2⁹,2¹⁰,2^{10a},2¹¹,2¹²,2¹³,2^{13a},4¹,4²,4³,4⁴,4⁵,4⁶- icosahydro-1¹H-6-oxa-2(2,5)-pyrrolo[2,3-k][1,2,4]triazolo[5,1-g][1,4,8]triazacyclotridecina-1(5,3)-indola-4(1,3)-pyridazinacyclononaphane-2¹³-carboxylate (95 mg, crude) in DMF (2.0 mL) stirred at room temperature was added CsF (61 mg, 0.40 mmol). The resulting mixture was stirred for 1 h, warmed to 60° C. for 3 h, and was then quenched at 0° C. by the addition of H₂O (5 mL). The aqueous mixture was extracted with EtOAc (3×5 mL) and the combined organic extracts were washed with brine (3×30 mL), dried over Na₂SO₄, filtered, and concentrated under reduced pressure. Purification by normal phase prep-TLC afforded tert-butyl (2⁵S,2⁸S,2^{10a}S,2^{13a}R,4³S,Z)-1¹-ethyl-2⁸-isopropyl-1²-(2-((S)-1-methoxyethyl)-5-(piperazin-1-yl)pyridin-3-yl)-2⁹,8,8-trimethyl-2⁷,2¹⁰,3,5-tetraoxo-2⁴,2⁵,2⁶,2⁷,2⁸,2⁹,2¹⁰,2^{10a},2¹¹,2¹²,2¹³,2^{13a},2¹⁴,2¹⁵,4¹,4²,4³,4⁴,4⁵,4⁶-icosahydro-1¹H-6-oxa-2(2,5)-pyrrolo[2,3-k][1,2,4]triazolo[5,1-g][1,4,8]triazacyclotridecina-1(5,3)-indola-4(1,3)-pyridazinacyclononaphane-2¹³-carboxylate (79 mg, 75% yield over 2 steps) as a light brown solid. LCMS (ESI) m/z: [M+H] calcd for C₅₅H₇₈N₁₂O₈: 1035.6; found 1035.6.

Step 18. To a solution of tert-butyl (2⁵S,2⁸S,2^{10a}S,2^{13a}R,4³S,Z)-1¹-ethyl-2⁸-isopropyl-1²-(2-((S)-1-methoxyethyl)-5-(piperazin-1-yl)pyridin-3-yl)-2⁹,8,8-trimethyl-2⁷,2¹⁰,3,5-tetraoxo-2⁴,2⁵,2⁶,2⁷,2⁸,2⁹,2¹⁰,2^{10a},2¹¹,2¹²,2¹³,2^{13a},2¹⁴,2¹⁵,4¹,4²,4³,4⁴,4⁵,4⁶-icosahydro-1¹H-6-oxa-2(2,5)-pyrrolo[2,3-k][1,2,4]triazolo[5,1-g][1,4,8]triazacyclotridecina-1(5,3)-indola-4(1,3)-pyridazinacyclononaphane-2¹³-carboxylate (77 mg, 0.074 mmol) and (1-ethoxycyclopropoxy)trimethylsilane (38.9 mg, 0.222 mmol) in IPA (1.0 mL) stirred at room temperature were added acetic acid (8.93 mg, 0.148 mmol) and NaBH₃CN (14.0 mg, 0.222 mmol). The resulting mixture was stirred for 3 h at 60° C. and was then quenched at 0° C. by the addition of H₂O (5 mL). The aqueous mixture was extracted with EtOAc (3×10 mL) and the combined organic extracts were dried over Na₂SO₄, filtered, and concentrated under reduced pressure. Purification by normal phase prep-TLC afforded tert-butyl (2⁵S,2⁸S,2^{10a}S,2^{13a}R,4³S,Z)-1²-(5-(4-cyclopropylpiperazin-1-yl)-2-((S)-1-methoxyethyl)pyridin-3-yl)-1¹-ethyl-2⁸-isopropyl-2⁹,8,8-trimethyl-2⁷,2¹⁰,3,5-tetraoxo-2⁴,2⁵,2⁶,2⁷,2⁸,2⁹,2¹⁰,2^{10a},2¹¹,2¹²,2¹³,2^{13a},2¹⁴,2¹⁵,4¹,4²,4³,4⁴,4⁵,4⁶-icosahydro-1¹H-6-oxa-2(2,5)-pyrrolo[2,3-k][1,2,4]triazolo[5,1-g][1,4,8]triazacyclotridecina-1 (5,3)-indola-4(1,3)-pyridazinacyclononaphane-2¹³-carboxylate (50 mg, 63% yield) as a light brown solid. LCMS (ESI) m/z: [M+H] calcd for C₅₈H₈₂N₁₂O₈: 1075.7; found 1075.5.

Step 19. To a solution of tert-butyl (2⁵S,2⁸S,2^{10a}S,2^{13a}R,4³S,Z)-1²-(5-(4-cyclopropylpiperazin-1-yl)-2-((S)-1-methoxyethyl)pyridin-3-yl)-1¹-ethyl-2⁸-isopropyl-2⁹,8,8-trimethyl-2⁷,2¹⁰,3,5-tetraoxo-2⁴,2⁵,2⁶,2⁷,2⁸,2⁹,2¹⁰,2^{10a},2¹¹,2¹²,2¹³,2^{13a},2¹⁴,2¹⁵,4¹,4²,4³,4⁴,4⁵,4⁶-icosahydro-1¹H-6-oxa-2(2,5)-pyrrolo[2,3-k][1,2,4]triazolo[5,1-g][1,4,8]triazacyclotridecina-1 (5,3)-indola-4(1,3)-pyridazinacyclononaphane-2¹³-carboxylate (50 mg, 0.046 mmol) in DCM (1.0 mL) stirred at room temperature was added TFA (200 μL).

The resulting mixture was stirred for 2 h, diluted with DCM (10 mL), and concentrated under reduced pressure to afford (2⁵S,2⁸S,2^{10a}S,2^{13a}R,4³S,Z)-1²-(5-(4-cyclopropylpiperazin-1-yl)-2-((S)-1-methoxyethyl)pyridin-3-yl)-1¹-ethyl-2⁸-isopropyl-2⁹,8,8-trimethyl-2⁴,2⁵,2⁶,2⁷,2⁸,2⁹,2¹⁰,2^{10a},2¹¹,2¹²,2¹³,2^{13a},2¹⁴,2¹⁵,4¹,4²,4³,4⁴,4⁵,4⁶-icosahydro-1¹H-6-oxa-2(2,5)-pyrrolo[2,3-k][1,2,4]triazolo[5,1-g][1,4,8]triazacyclotridecina-1 (5,3)-indola-4(1,3)-pyridazinacyclononaphane-2⁷,2¹⁰,3,5-tetraone (50 mg, crude) which was taken directly to the next reaction without further purification. LCMS (ESI) m/z: [M+H] calcd for C₅₃H₇₄N₁₂O₆: 975.6; found 975.5.

Step 20. To a solution of (2⁵S,2⁸S,2^{10a}S,2^{13a}R,4³S,Z)-1²-(5-(4-cyclopropylpiperazin-1-yl)-2-((S)-1-methoxyethyl)pyridin-3-yl)-1¹-ethyl-2⁸-isopropyl-2⁹,8,8-trimethyl-2⁴,2⁵,2⁶,2⁷,2⁸,2⁹,2¹⁰,2^{10a},2¹¹,2¹²,2¹³,2^{13a},2¹⁴,2¹⁵,4¹,4²,4³,4⁴,4⁵,4⁶-icosahydro-1¹H-6-oxa-2(2,5)-pyrrolo[2,3-k][1,2,4]triazolo[5,1-g][1,4,8]triazacyclotridecina-1 (5,3)-indola-4(1,3)-pyridazinacyclononaphane-2⁷,2¹⁰,3,5-tetraone (50 mg, crude) and DIPEA (66.3 mg, 0.510 mmol) in DMF (2.0 mL) stirred at 0° C. were added 2-fluoro-2-methylpropanoic acid (10.9 mg, 0.102 mmol) and COMU (43.9 mg, 0.102 mmol). The resulting mixture was stirred for 2 h and was then quenched at 0° C. by the addition of H₂O (5 mL). The aqueous mixture was extracted with EtOAc (3×5 mL), and the combined organic extracts were washed with brine (3×30 mL), dried over Na₂SO₄, filtered, and concentrated under reduced pressure. Purification by reversed phase prep-HPLC afforded (2⁵S,2⁸S,2^{10a}S,2^{13a}R,4³S,Z)-12-(5-(4-cyclopropylpiperazin-1-yl)-2—((S)-1-methoxyethyl)pyridin-3-yl)-1¹-ethyl-2¹³-(2-fluoro-2-methylpropanoyl)-2⁸-isopropyl-2⁹,8,8-trimethyl-2⁴,2⁵,2⁶,2⁷,2⁸,2⁹,2¹⁰,2^{10a},2¹¹,2¹²,2¹³,2^{13a},2¹⁴,2¹⁵,4¹,4²,4³,4⁴,4⁵,4⁶-icosahydro-1¹H-6-oxa-2(2,5)-pyrrolo[2,3-k][1,2,4]triazolo[5,1-g][1,4,8]triazacyclotridecina-1 (5,3)-indola-4(1,3)-pyridazinacyclononaphane-2⁷,2¹⁰,3,5-tetraone (19.2 mg, 39% yield over 2 steps) as a white solid. LCMS (ESI) m/z: [M+H] calcd for C₅₇H₇₉FN₁₂O₇: 1063.6; found 1063.8. ¹H NMR (300 MHz, DMSO-d₆) δ 8.33 (d, J=8.5 Hz, 1H), 8.25 (d, J=1.6 Hz, 1H), 8.19 (d, J=2.8 Hz, 1H), 7.50 (dd, J=8.6, 1.5 Hz, 1H), 7.27 (d, J=8.6 Hz, 1H), 6.94 (d, J=2.8 Hz, 1H), 5.12 (t, J=7.8 Hz, 1H), 4.73 (d, J=11.9 Hz, 1H), 4.54 (d, J=11.2 Hz, 1H), 4.20 (t, J=12.2 Hz, 1H), 3.90 (m, 8H), 3.44 (m, 1H), 3.31 (d, J=10.9 Hz, 2H), 3.18 (s, 1H), 2.98 (d, J=8.7 Hz, 8H), 2.79 (d, J=14.3 Hz, 2H), 2.69 (s, 3H), 2.58-2.37 (m, 6H), 2.11 (d, J=14.2 Hz, 1H), 1.92 (d, J=9.7 Hz, 3H), 1.84-1.70 (m, 1H), 1.57 (s, 3H), 1.46-1.35 (m, 4H), 1.30 (d, J=1.9 Hz, 3H), 1.12-0.95 (m, 4H), 0.74 (s, 3H), 0.68-0.48 (m, 9H), 0.18 (dd, J=6.6, 4.3 Hz, 2H), 0.10-0.02 (m, 5H).

### Example A418. Synthesis of (3aS,6S,9S,15S,32aS)-6-((R)-1-cyclopropylethyl)-21-(5-(4-cyclopropylpiperazin-1-yl)-2-((S)-1-methoxyethyl)pyridin-3-yl)-22-ethyl-1-(2-fluoro-2-methylpropanoyl)-5,19,19-trimethyl-3,3a,5,6,8,9,14,15,18,19,20,22,32,32a-tetradecahydro-1H,12H-11,15-epimino-23,25-etheno-9,28-methano-26,30-(metheno)dipyrrolo[2,3-c:3′,4′-v][1,18]dioxa[6,9,12]triazacyclotriacontine-4,7,10,16(2H,13H)-tetraone

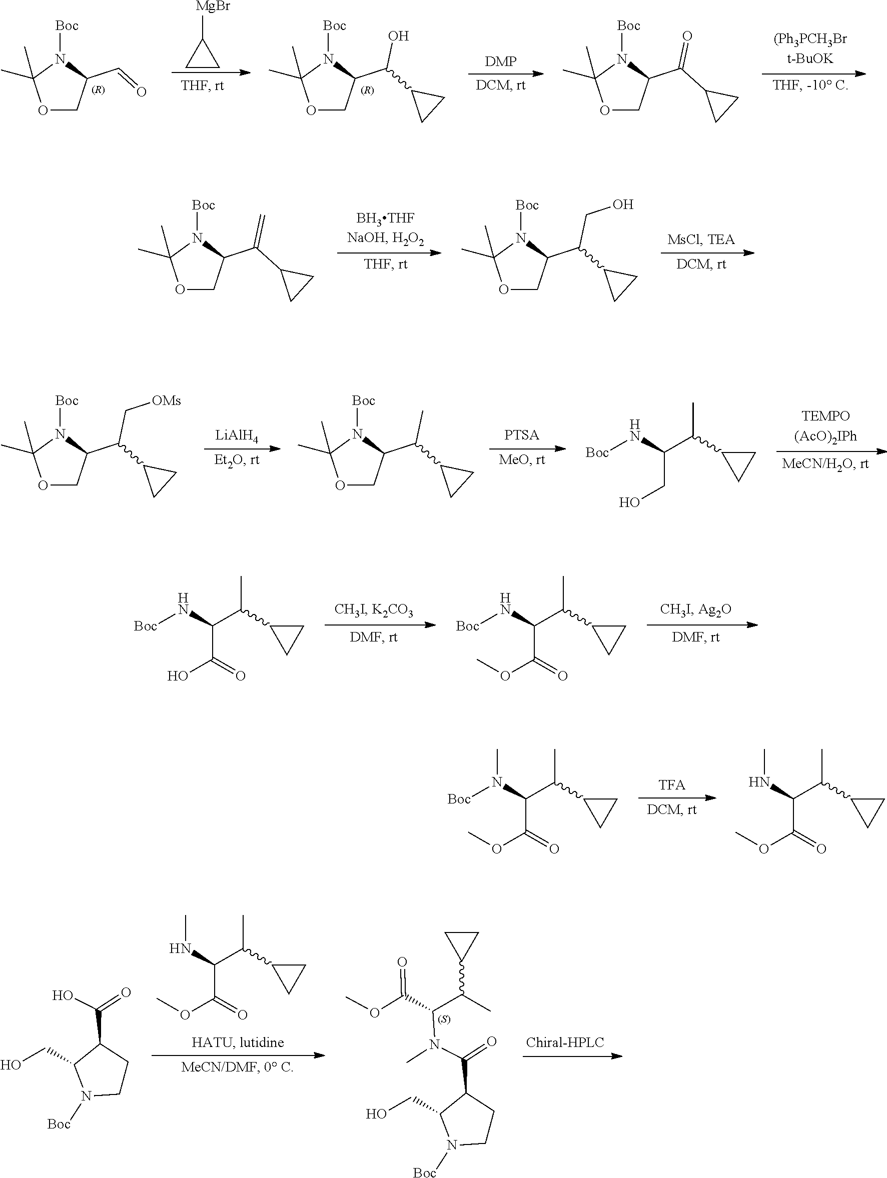

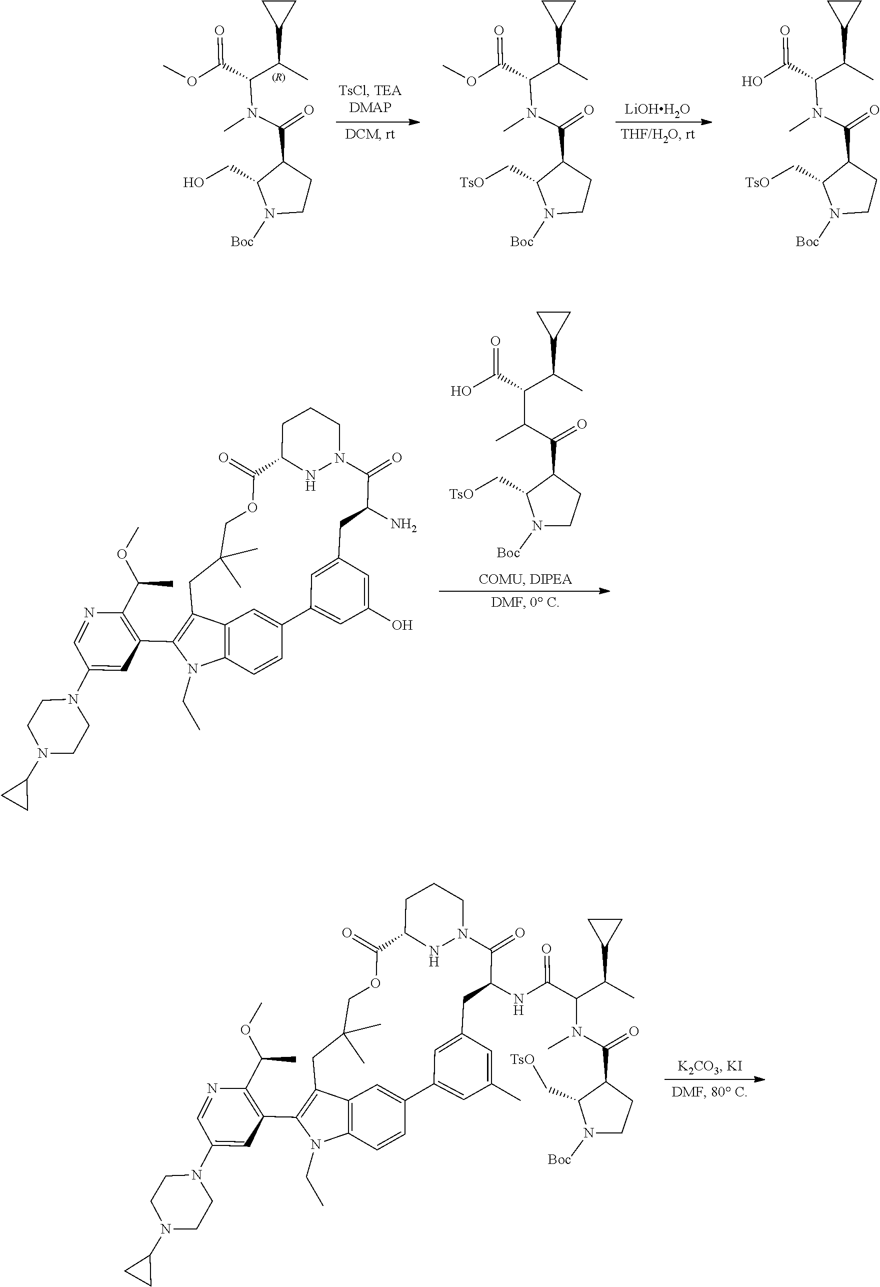

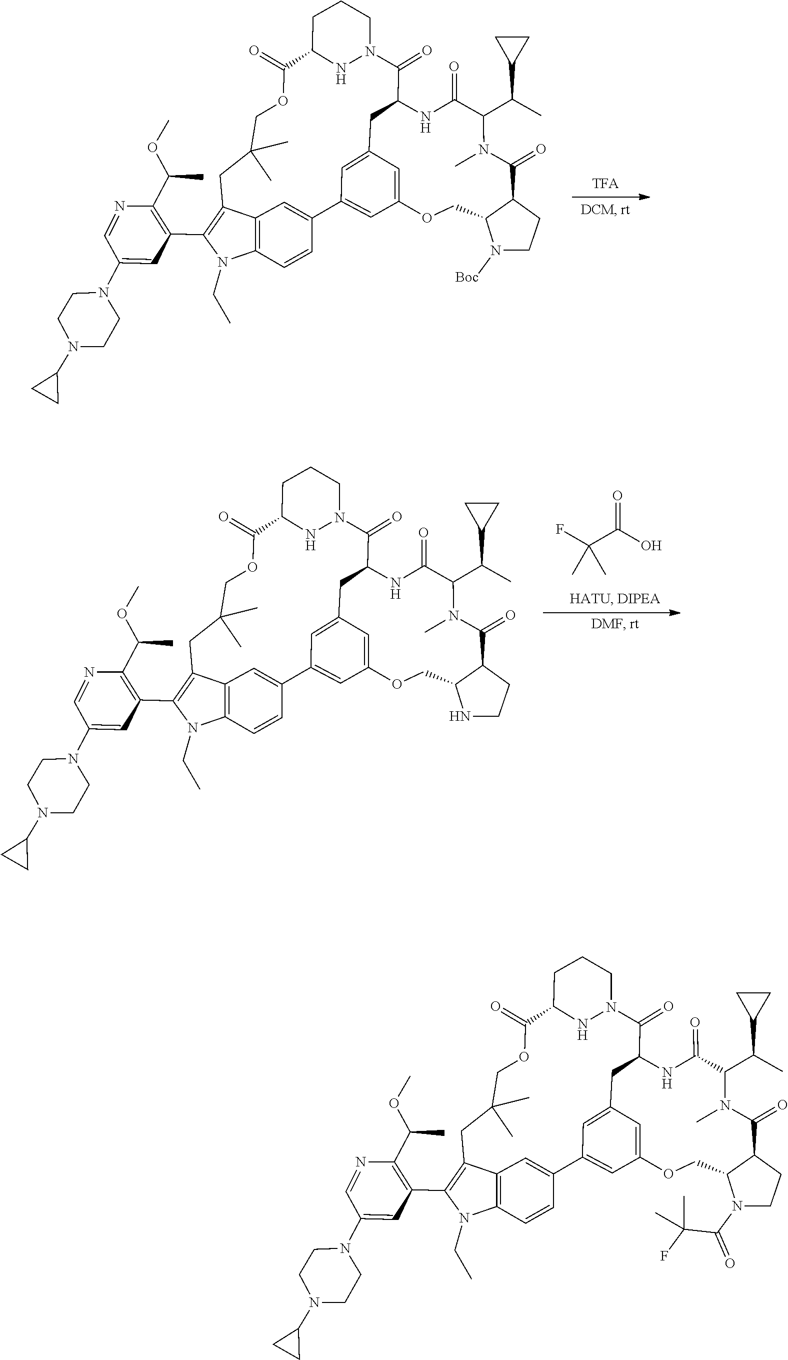

Step 1. To a stirred solution of tert-butyl (4R)-4-formyl-2,2-dimethyl-1,3-oxazolidine-3-carboxylate (20.0 g, 87.2 mmol) in THF (200 mL) was added cyclopropylmagnesium bromide (174 mL, 174 mmol) dropwise at −78° C. under an atmosphere of N₂. The resulting mixture was stirred overnight at room temperature and was then quenched by the addition of H₂O (200 mL) at 0° C. The aqueous mixture was extracted with EtOAc (3×100 mL), and the combined organic extracts were dried over Na₂SO₄, filtered, and concentrated under reduced pressure. The residue was purified by normal phase chromatography to afford tert-butyl (4R)-4-[cyclopropyl(hydroxy)methyl]-2,2-dimethyl-1,3-oxazolidine-3-carboxylate (23.3 g, 98% yield) as a yellow oil. LCMS (ESI) m/z: [M+H] calcd for C₁₄H₂₅NO₄: 272.2; found 272.2.

Step 2. To a stirred solution of tert-butyl (4R)-4-[cyclopropyl(hydroxy)methyl]-2,2-dimethyl-1,3-oxazolidine-3-carboxylate (23.3 g, 85.9 mmol) in DCM (200 mL) was added Dess-Martin periodinane (54.6 g, 129 mmol) at 0° C. The resulting mixture was stirred for 3 h at room temperature and was then quenched by the addition of sat. aq. Na₂S₂O₃ (100 mL) at 0° C. The aqueous layer was extracted with DCM (2×100 mL) and the combined organic extracts were washed with sat. aq. NaHCO₃ (2×200 mL), dried over Na₂SO₄, filtered, and concentrated under reduced pressure. The crude residue was purified by normal phase chromatography to afford tert-butyl (4R)-4-cyclopropanecarbonyl-2,2-dimethyl-1,3-oxazolidine-3-carboxylate (16.3 g, 70% yield) as a white solid. LCMS (ESI) m/z: [M+H] calcd for C₁₄H₂₃NO₄: 270.2; found 270.2.

Step 3. To a stirred solution of methyltriphenylphosphonium bromide (53.7 g, 150 mmol) in THE (100 mL) was added t-BuOK (150 mL, 150 mmol) dropwise at −10° C. under an atmosphere of N₂. The resulting mixture was stirred at −10° C. under an atmosphere of N₂ for 1 h, after which time a solution of tert-butyl (4R)-4-cyclopropanecarbonyl-2,2-dimethyl-1,3-oxazolidine-3-carboxylate (16.2 g, 60.1 mmol) in THE (60 mL) was added dropwise at −10° C. The resulting mixture was stirred for 1 h at −10° C. and was then quenched by the addition of sat. aq. NH₄Cl (100 mL) at 0° C. The aqueous mixture was extracted with EtOAc (3×100 mL) and the combined organic extracts were dried over Na₂SO₄, filtered, and concentrated under reduced pressure. The residue was purified by normal phase chromatography to afford tert-butyl (4S)-4-(1-cyclopropylethenyl)-2,2-dimethyl-1,3-oxazolidine-3-carboxylate (13.7 g, 85% yield) as a colorless oil. LCMS (ESI) m/z: [M−C₄H₈+H] calcd for C₁₅H₂₅NO₃: 212.1 found 212.0.

Step 4. To a stirred solution of tert-butyl (4S)-4-(1-cyclopropylethenyl)-2,2-dimethyl-1,3-oxazolidine-3-carboxylate (13.6 g, 50.9 mmol) in THE (200 mL) was added BH₃·THF (254 mL, 254 mmol) dropwise at 0° C. The resulting mixture was stirred for 1 h at 0° C. and was then treated with 1 M aq. NaOH (102 mL, 102 mmol) and 30% H₂O₂ (11.5 g, 102 mmol) dropwise at 0° C. The reaction mixture was stirred for 2 days at room temperature, quenched by the addition of MeOH at 0° C., and diluted with H₂O (200 mL). The aqueous mixture was extracted with EtOAc (3×100 mL) and the combined organic extracts were dried over Na₂SO₄, filtered, and concentrated under reduced pressure. The residue was purified by normal phase chromatography to afford tert-butyl (4S)-4-(1-cyclopropyl-2-hydroxyethyl)-2,2-dimethyl-1,3-oxazolidine-3-carboxylate (13.6 g, 94% yield) as a yellow oil. LCMS (ESI) m/z: [M+H] calcd for C₁₅H₂₇NO₄: 286.2; found 286.1.

Step 5. To a stirred solution of tert-butyl (4S)-4-(1-cyclopropyl-2-hydroxyethyl)-2,2-dimethyl-1,3-oxazolidine-3-carboxylate (14.1 g, 49.4 mmol) and TEA (20.6 mL, 148 mmol) in DCM (100 mL) was added MsCl (8.49 g, 74.1 mmol) dropwise at 0° C. The resulting mixture was stirred for 1 h at room temperature and was then quenched by the addition of H₂O (100 mL) at 0° C. The aqueous layer was extracted with DCM (3×100 mL) and the combined organic extracts were dried over Na₂SO₄, filtered, and concentrated under reduced pressure to afford tert-butyl (4S)-4-[1-cyclopropyl-2-(methanesulfonyloxy)ethyl]-2,2-dimethyl-1,3-oxazolidine-3-carboxylate (17.5 g, crude), which was used without further purification. LCMS (ESI) m/z: [M−C₄H₈+H] calcd for C₁₆H₂₉NO₆S: 308.1; found 308.1.

Step 6. To a stirred solution of tert-butyl (4S)-4-[1-cyclopropyl-2-(methanesulfonyloxy)ethyl]-2,2-dimethyl-1,3-oxazolidine-3-carboxylate (16.5 g, crude) in Et₂₀ (150 mL) was added 2 M LiAlH₄ (34.1 mL, 68.1 mmol) dropwise at 0° C. The resulting mixture was stirred overnight at room temperature and was then quenched by the dropwise addition of EtOAc at 0° C., treated with Na₂SO₄·10H₂O, stirred for an additional 30 min at room temperature, and filtered. The filter cake was washed with EtOAc (3×50 mL), the filtrate concentrated under reduced pressure, and the residue purified by reversed phase chromatography to afford tert-butyl (4S)-4-(1-cyclopropylethyl)-2,2-dimethyl-1,3-oxazolidine-3-carboxylate (6.10 g, 49% yield over 2 steps) as a yellow oil. LCMS (ESI) m/z: [M−C₄H₈+H] calcd for C₁₅H₂₇NO₃: 214.1; found 214.2.

Step 7. To a stirred solution of tert-butyl (4S)-4-(1-cyclopropylethyl)-2,2-dimethyl-1,3-oxazolidine-3-carboxylate (5.10 g, 18.9 mmol) in MeOH (50 mL) was added PTSA (815 mg, 4.73 mmol) at 0° C. The resulting mixture was stirred for overnight at room temperature and was then basified to pH 8 with sat aq. NaHCO₃, concentrated under reduced pressure, and diluted with H₂O (100 mL). The aqueous mixture was extracted with EtOAc (3×100 mL), dried over anhydrous Na₂SO₄, filtered, and concentrated under reduced pressure to afford tert-butyl N-[(2S)-3-cyclopropyl-1-hydroxybutan-2-yl]carbamate (3.00 g, crude), which was used without further purification. LCMS (ESI) m/z: [M−C₄H₈+H] calcd for C₁₂H₂₃NO₃: 174.1 found 174.0.

Step 8. To a stirred solution of tert-butyl N-[(2S)-3-cyclopropyl-1-hydroxybutan-2-yl]carbamate (3.00 g, crude) in ACN (15 mL) and H₂O (15 mL) were added (diacetoxyiodo)benzene (9.27 g, 28.8 mmol) and TEMPO (613 mg, 3.93 mmol) at 0° C. The resulting mixture was stirred overnight at 25° C. and was then concentrated under reduced pressure. The aqueous mixture was acidified to pH 6 with 1 M aq. HCl, extracted with EtOAc (3×20 mL) and the combined organic extracts were dried over Na₂SO₄, filtered, and concentrated under reduced pressure. The residue was purified by reversed phase chromatography to afford (2S)-2-[(tert-butoxycarbonyl)amino]-3-cyclopropylbutanoic acid (2.68 g, 58% yield over 2 steps) as a yellow solid. LCMS (ESI) m/z: [M−C₄H₈+H] calcd for C₁₂H₂₁NO₄: 188.1; found 188.1.

Step 9. To a stirred solution of (2S)-2-[(tert-butoxycarbonyl)amino]-3-cyclopropylbutanoic acid (2.63 g, 10.8 mmol) in DMF (15 mL) was added K₂CO₃ (3.73 g, 27.0 mmol) at 0° C. The resulting mixture was stirred for 15 min at 0° C. under air atmosphere and was then treated with a solution of CH₃I (5.37 g, 37.8 mmol) in DMF (5 mL) 0° C. The reaction mixture was stirred overnight at room temperature and was then quenched by the addition of H₂O (20 mL) at 0° C. The resulting aqueous mixture was extracted with EtOAc (3×10 mL) and the combined organic extracts were washed with brine (3×60 mL), dried over Na₂SO₄, filtered, and concentrated under reduced pressure. The residue was purified by normal phase chromatography to afford methyl (2S)-2-[(tert-butoxycarbonyl)amino]-3-cyclopropylbutanoate (2.30 g, 83% yield) as a yellow oil. LCMS (ESI) m/z: [M−C₄H₈+H] calcd for C₁₃H₂₃NO₄: 202.1; found 202.1.

Step 10. To a stirred solution of methyl (2S)-2-[(tert-butoxycarbonyl)amino]-3-cyclopropylbutanoate (2.30 g, 8.94 mmol) and Ag₂O (10.4 g, 44.7 mmol) in DMF (20 mL) was added Mel (19.0 g, 134 mmol) dropwise at 0° C. The resulting mixture was stirred for 4 days in the absence of light at room temperature and was then filtered. The filter cake was washed with EtOAc (3×10 mL) and the filtrate was washed with brine (3×70 mL), dried over Na₂SO₄, filtered, and concentrated under reduced pressure. The residue was purified by reversed phase chromatography to afford methyl (2S)-2-[(tert-butoxycarbonyl)(methyl)amino]-3-cyclopropylbutanoate (1.60 g, 66% yield) as a yellow oil. LCMS (ESI) m/z: [M−C₄H₈+H] calcd for C₁₄H₂₅NO₄: 216.1; found 216.2.

Step 11. To a stirred solution of methyl (2S)-2-((tert-butoxycarbonyl)(methyl)amino)-3-cyclopropylbutanoate (1.60 g, 5.90 mmol) in DCM (15 mL) was added TFA (3 mL) at 0° C. The resulting mixture was stirred for 1 h at room temperature and was then concentrated under reduced pressure to yield methyl (2S)-3-cyclopropyl-2-(methylamino)butanoate (2.09 g, crude), which was used without further purification. LCMS (ESI) m/z: [M+H] calcd for C₉H₁₇NO₂: 172.1 found 172.1.

Step 12. To a stirred solution of (2S,3S)-1-(tert-butoxycarbonyl)-2-(hydroxymethyl)pyrrolidine-3-carboxylic acid (786 mg, 3.21 mmol) in ACN (16 mL) were added methyl (2S)-3-cyclopropyl-2-(methylamino)butanoate (988 mg, crude) and 2,6-lutidine (3.43 g, 32.1 mmol) at −20° C. followed by a solution of HATU (1.71 g, 4.49 mmol) in DMF (1.6 mL) at −20° C. The resulting mixture was stirred for 2 h at 0° C. and the reaction was then quenched by the addition of salted ice at −20° C. The aqueous mixture was acidified to pH 5 with 2 M aq. HCl, extracted with EtOAc (2×50 mL) and the combined organic extracts were washed with brine (3×10 mL), dried over Na₂SO₄, filtered, and concentrated under reduced pressure. The residue was purified by reversed phase chromatography to yield tert-butyl (2S,3S)-3-(((2S)-3-cyclopropyl-1-methoxy-1-oxobutan-2-yl)(methyl)carbamoyl)-2-(hydroxymethyl)pyrrolidine-1-carboxylate (461 mg, 36% yield) as a colorless oil. LCMS (ESI) m/z: [M+H] calcd for C₂₀H₃₄N₂O₆: 399.2; found 399.2.

Step 13. tert-Butyl (2S,3S)-3-(((2S)-3-cyclopropyl-1-methoxy-1-oxobutan-2-yl) (methyl)carbamoyl)-2-(hydroxymethyl)pyrrolidine-1-carboxylate (461 mg, 1.16 mmol) was purified by chiral-HPLC (CHIRAL ART cellulose-SC column) to afford the faster eluting epimer tert-butyl (2S,3S)-3-{[(2S,3R)-3-cyclopropyl-1-methoxy-1-oxobutan-2-yl](methyl)carbamoyl}-2-(hydroxymethyl)pyrrolidine-1-carboxylate (161 mg, 35% yield) as a colorless oil followed by the slower eluting epimer tert-butyl (2S,3S)-3-{[(2S,3S)-3-cyclopropyl-1-methoxy-1-oxobutan-2-yl](methyl)carbamoyl}-2-(hydroxymethyl)pyrrolidine-1-carboxylate (182 mg, 39% yield) as a colorless oil. LCMS (ESI) m/z: [M+H] calcd for C₂₀H₃₄N₂O₆: 399.2; found 399.2.

Step 14. To a stirred solution of tert-butyl (2S,3S)-3-{[(2S,3R)-3-cyclopropyl-1-methoxy-1-oxobutan-2-yl](methyl)carbamoyl}-2-(hydroxymethyl)pyrrolidine-1-carboxylate (145 mg, 0.364 mmol) and TEA (221 mg, 2.18 mmol) in DCM (3 mL) were added DMAP (4.45 mg, 0.036 mmol) and a solution of TsCl (312 mg, 1.64 mmol) in DCM (2 mL) at 0° C. The resulting mixture was stirred for 1 h at room temperature and was then quenched by the addition of H₂O (5 mL) at 0° C. The aqueous mixture was extracted with DCM (2×5 mL) and the combined organic extracts were dried over Na₂SO₄, filtered, and concentrated under reduced pressure. The residue was purified by prep-TLC to afford tert-butyl (2S,3S)-3—{[(2S,3R)-3-cyclopropyl-1-methoxy-1-oxobutan-2-yl](methyl)carbamoyl}-2-{[(4-methylbenzenesulfonyl)oxy]methyl}pyrrolidine-1-carboxylate (146 mg, 73% yield) as a yellow oil. LCMS (ESI) m/z: [M−C₄H₈+H] calcd for C₂₇H₄₀N₂O₈S: 497.2; found 497.3.

Step 15. To a stirred solution of tert-butyl (2S,3S)-3-{[(2S,3R)-3-cyclopropyl-1-methoxy-1-oxobutan-2-yl](methyl)carbamoyl}-2-{[(4-methylbenzenesulfonyl)oxy]methyl}pyrrolidine-1-carboxylate (136 mg, 0.246 mmol) in THE (4 mL) was added a solution of LiOH·H₂O (20.7 mg, 0.492 mmol) in H₂O (4 mL) at 0° C. The resulting mixture was stirred overnight at room temperature and was acidified to pH 5 by the addition of 2 M aq. HCl. The aqueous mixture was extracted with EtOAc (3×5 mL) and the combined organic extracts were dried over Na₂SO₄, filtered, and concentrated under reduced pressure to afford (2S,3R)-2-{1-[(2S,3S)-1-(tert-butoxycarbonyl)-2-{[(4-methylbenzenesulfonyl)oxy]methyl}pyrrolidin-3-yl]-N-methylformamido}-3-cyclopropylbutanoic acid (149 mg, crude), which was used without further purification. LCMS (ESI) m/z: [M−C₄H₈+H] calcd for C₂₆H₃₈N₂O₈S: 483.1; found 483.2.

Step 16. To a stirred solution of (6³S,4S)-4-amino-1²-(5-(4-cyclopropylpiperazin-1-yl)-2-((S)-1-methoxyethyl)pyridin-3-yl)-1¹-ethyl-2⁵-hydroxy-10,10-dimethyl-6¹,6²,6³,6⁴,6⁵,6⁶-hexahydro-1 ¹H-8-oxa-1(5,3)-indola-6(1,3)-pyridazina-2(1,3)-benzenacycloundecaphane-5,7-dione (66.0 mg, 0.086 mmol) and (2S,3R)-2-{1-[(2S,3S)-1-(tert-butoxycarbonyl)-2-{[(4-methylbenzenesulfonyl)oxy]methyl}pyrrolidin-3-yl]-N-methylformamido}-3-cyclopropylbutanoic acid (60.5 mg, crude) in DMF (2 mL) were added DIPEA (447 mg, 3.44 mmol) and COMU (48.1 mg, 0.112 mmol) at 0° C. The resulting mixture was stirred for 1 h at 0° C. and was then quenched by the addition of H₂O (3 mL) at 0° C. The aqueous mixture was extracted with EtOAc (3×3 mL) and the combined organic extracts were washed with brine (3×10 mL), dried over Na₂SO₄, filtered, and concentrated under reduced pressure. The residue was purified by prep-TLC to afford tert-butyl (2S,3S)-3-(((2S,3R)-3-cyclopropyl-1-(((6³S,4S)-1²-(5-(4-cyclopropylpiperazin-1-yl)-2-((S)-1-methoxyethyl)pyridin-3-yl)-1¹-ethyl-2⁵-hydroxy-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)amino)-1-oxobutan-2-yl)(methyl)carbamoyl)-2-((tosyloxy)methyl)pyrrolidine-1-carboxylate (55 mg, 50% yield) as a yellow solid. LCMS (ESI) m/z: [M+H] calcd for C₇₀H₉₃N₉O₁₂S: 1284.7; found 1284.7.

Step 17. A mixture tert-butyl (2S,3S)-3-(((2S,3R)-3-cyclopropyl-1-(((6³S,4S)-12-(5-(4-cyclopropylpiperazin-1-yl)-2-((S)-1-methoxyethyl)pyridin-3-yl)-1¹-ethyl-2⁵-hydroxy-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)amino)-1-oxobutan-2-yl)(methyl)carbamoyl)-2-((tosyloxy)methyl)pyrrolidine-1-carboxylate (50.0 mg, 0.039 mmol), K₂CO₃ (53.8 mg, 0.390 mmol) and KI (6.46 mg, 0.039 mmol) in DMF (5 mL) was stirred for 1 h at 80° C. The reaction was quenched by the addition of cold H₂O (5 mL) at 0° C. and the resulting mixture was extracted with EtOAc (3×5 mL), washed with brine (3×20 mL), dried over Na₂SO₄, filtered, and concentrated under reduced pressure. The residue was purified by prep-TLC to afford tert-butyl (3aS,6S,9S,15S,32aS)-6-((R)-1-cyclopropylethyl)-21-(5-(4-cyclopropylpiperazin-1-yl)-2-((S)-1-methoxyethyl)pyridin-3-yl)-22-ethyl-5,19,19-trimethyl-4,7,10,16-tetraoxo-2,3,3a,4,5,6,7,8,9,10,13,14,15,16,18,19,20,22,32,32a-icosahydro-1H,12H-11,15-epimino-23,25-etheno-9,28-methano-26,30-(metheno)dipyrrolo[2,3-c:3′,4′-v][1,18]dioxa[6,9,12]triazacyclotriacontine-1-carboxylate (40 mg, 92% yield) as a yellow solid. LCMS (ESI) m/z: [M+H] calcd for C₆₃H₈₅N₉O₉: 1112.6; found 1112.7.

Step 18. To a stirred solution of tert-butyl (3aS,6S,9S,15S,32aS)-6-((R)-1-cyclopropylethyl)-21-(5-(4-cyclopropylpiperazin-1-yl)-2-((S)-1-methoxyethyl)pyridin-3-yl)-22-ethyl-5,19,19-trimethyl-4,7,10,16-tetraoxo-2,3,3a,4,5,6,7,8,9,10,13,14,15,16,18,19,20,22,32,32a-icosahydro-1H,12H-11,15-epimino-23,25-etheno-9,28-methano-26,30-(metheno)dipyrrolo[2,3-c:3′,4′-v][1,18]dioxa[6,9,12]triazacyclotriacontine-1-carboxylate (41 mg, 0.037 mmol) in DCM (2 mL) was added TFA (0.40 mL, 5.4 mmol) at 0° C. The reaction mixture was stirred for 1 hour at room temperature and was then concentrated under reduced pressure to afford (3aS,6S,9S,15S,32aS)-6-((R)-1-cyclopropylethyl)-21-(5-(4-cyclopropylpiperazin-1-yl)-2-((S)-1-methoxyethyl)pyridin-3-yl)-22-ethyl-5,19,19-trimethyl-3,3a,5,6,8,9,14,15,18,19,20,22,32,32a-tetradecahydro-1H,12H-11,15-epimino-23,25-etheno-9,28-methano-26,30-(metheno)dipyrrolo[2,3-c:3′,4′-v][1,18]dioxa[6,9,12]triazacyclotriacontine-4,7,10,16(2H,13H)-tetraone (51 mg, crude), which was used without further purification. LCMS (ESI) m/z: [M+H] calcd for C₅₈H₇₇N₉O₇: 1012.6; found 1012.7.

Step 19. To a stirred solution of (3aS,6S,9S,15S,32aS)-6-((R)-1-cyclopropylethyl)-21-(5-(4-cyclopropylpiperazin-1-yl)-2-((S)-1-methoxyethyl)pyridin-3-yl)-22-ethyl-5,19,19-trimethyl-3,3a,5,6,8,9,14,15,18,19,20,22,32,32a-tetradecahydro-1H,12H-11,15-epimino-23,25-etheno-9,28-methano-26,30-(metheno)dipyrrolo[2,3-c:3′,4′-v][1,18]dioxa[6,9,12]triazacyclotriacontine-4,7,10,16(2H,13H)-tetraone (46 mg, crude) and 2-fluoro-2-methylpropanoic acid (9.64 mg, 0.090 mmol) in DMF (3 mL) were added DIPEA (58.7 mg, 0.450 mmol) and HATU (34.6 mg, 0.090 mmol) at 0° C. The resulting mixture was stirred for 40 min at room temperature and was then quenched by the addition of H₂O (5 mL) at 0° C. The aqueous mixture was extracted with EtOAc (2×3 mL) and the combined organic extracts were washed with brine (3×10 mL), dried over Na₂SO₄, filtered, and concentrated under reduced pressure. The residue was purified by reversed phase chromatography to afford (3aS,6S,9S,15S,32aS)-6-((R)-1-cyclopropylethyl)-21-(5-(4-cyclopropylpiperazin-1-yl)-2-((S)-1-methoxyethyl)pyridin-3-yl)-22-ethyl-1-(2-fluoro-2-methylpropanoyl)-5,19,19-trimethyl-3,3a,5,6,8,9,14,15,18,19,20,22,32,32a-tetradecahydro-1H,12H-11,15-epimino-23,25-etheno-9,28-methano-26,30-(metheno)dipyrrolo[2,3-c:3′,4′-v][1,18]dioxa[6,9,12]triazacyclotriacontine-4,7,10,16(2H,13H)-tetraone (8.0 mg, 22% yield over 2 steps) as a white solid. LCMS (ESI) m/z: [M+H] calcd for C₆₂H₈₂FN₉O₈: 1100.6; found 1100.8. ¹H NMR (400 MHz, DMSO-d₆) δ 8.46 (d, J=2.9 Hz, 1H), 8.17 (d, J=6.9 Hz, 1H), 7.68 (s, 1H), 7.57 (d, J=8.5 Hz, 1H), 7.43 (d, J=8.5 Hz, 1H), 7.40-7.35 (m, 2H), 7.29-7.08 (m, 1H), 6.62 (s, 1H), 6.16 (s, 1H), 5.20-5.09 (m, 1H), 5.01-4.90 (m, 1H), 4.56-4.43 (m, 1H), 4.32-3.93 (m, 5H), 3.91-3.78 (m, 1H), 3.75-3.60 (m, 3H), 3.47-3.42 (m, 3H), 3.28-3.14 (m, 5H), 3.11-2.85 (m, 4H), 2.74 (s, 3H), 2.71-2.63 (m, 5H), 2.35-2.21 (m, 1H), 2.12-1.94 (m, 2H), 1.93-1.78 (m, 2H), 1.76-1.56 (m, 5H), 1.51 (s, 2H), 1.47 (d, J=6.7 Hz, 3H), 1.42 (s, 1H), 1.38 (d, J=6.3 Hz, 3H), 1.32-1.19 (m, 3H), 0.93 (s, 3H), 0.78 (d, J=6.7 Hz, 3H), 0.62-0.50 (m, 1H), 0.44 (s, 5H), 0.38-0.20 (m, 5H), −0.01 (s, 1H), −0.09 (s, 1H).

### Example A423. Synthesis of (3aS,6S,9S,15S,32aS)-22-ethyl-1-(2-fluoro-2-methylpropanoyl)-6-isopropyl-21-(2-((S)-1-methoxyethyl)-5-(piperazin-1-yl)pyridin-3-yl)-5,19,19-trimethyl-3,3a,5,6,8,9,14,15,18,19,20,22,32,32a-tetradecahydro-1H,12H-11,15-epimino-23,25-etheno-9,28-methano-26,30-(metheno)dipyrrolo[2,3-c:3′,4′-v][1,18]dioxa[6,9,12]triazacyclotriacontine-4,7,10,16(2H,13H)-tetraone

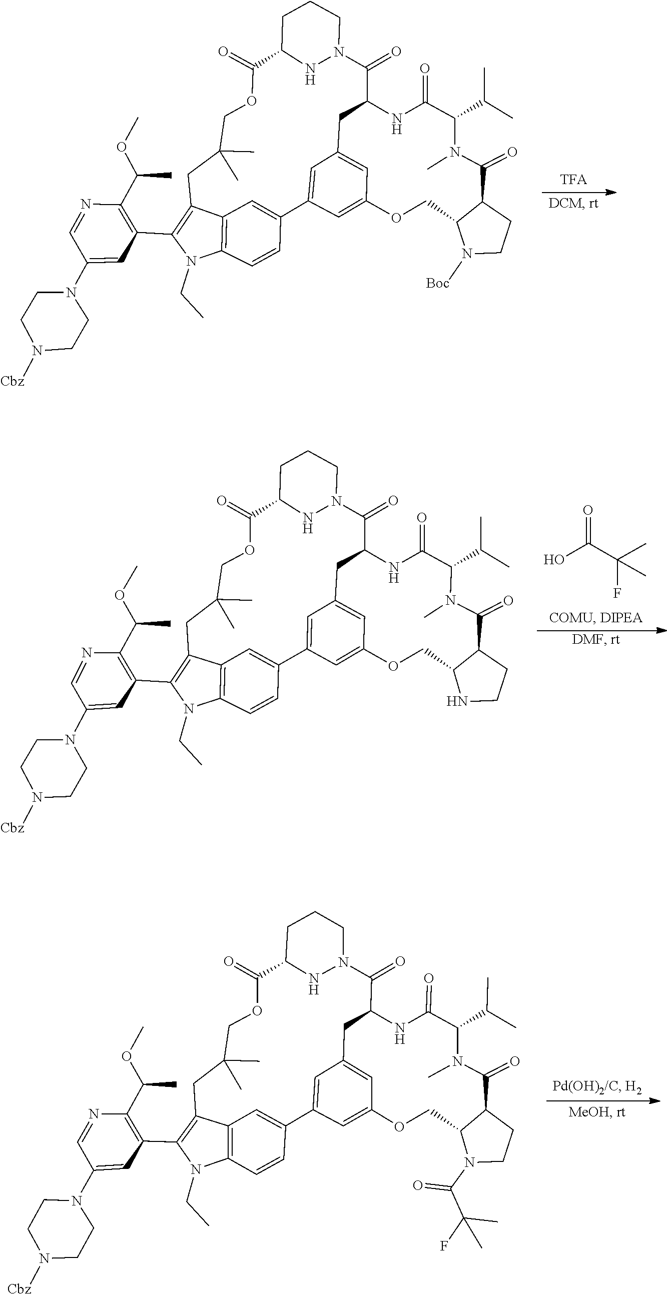

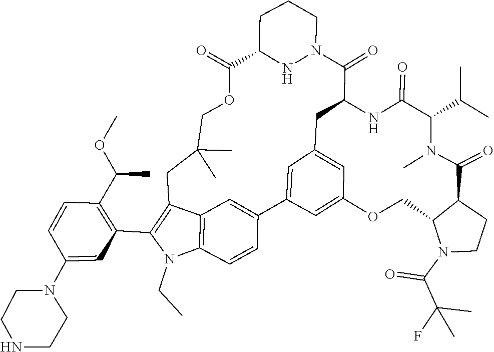

Step 1. To a solution of tert-butyl (3aS,6S,9S,15S,32aS)-21-(5-(4-((benzyloxy)carbonyl)piperazin-1-yl)-2-((S)-1-methoxyethyl)pyridin-3-yl)-22-ethyl-6-isopropyl-5,19,19-trimethyl-4,7,10,16-tetraoxo-2,3,3a,4,5,6,7,8,9,10,13,14,15,16,18,19,20,22,32,32a-icosahydro-1H,12H-11,15-epimino-23,25-etheno-9,28-methano-26,30-(metheno)dipyrrolo[2,3-c:3′,4′-v][1,18]dioxa[6,9,12]triazacyclotriacontine-1-carboxylate (400 mg, 0.339 mmol) in DCM (8.0 mL) stirred at 0° C. was added TFA (8.0 mL). The resulting mixture was stirred for 5 h at room temperature, diluted with DCM (5 mL), and basified to pH=10 by the addition of sat. aq. NaHCO₃. The aqueous phase was extracted with DCM (3×20 mL), and the combined organic layers were washed with brine (2×20 mL), dried over Na₂SO₄, filtered, and concentrated under reduced pressure to afford benzyl 4-(5-((3aS,6S,9S,15S,32aS)-22-ethyl-6-isopropyl-5,19,19-trimethyl-4,7,10,16-tetraoxo-2,3,3a,4,5,6,7,8,9,10,13,14,15,16,18,19,20,22,32,32a-icosahydro-1H,12H-11,15-epimino-23,25-etheno-9,28-methano-26,30-(metheno)dipyrrolo[2,3-c:3′,4′-v][1,18]dioxa[6,9,12]triazacyclotriacontin-21-yl)-6-((S)-1-methoxyethyl)pyridin-3-yl)piperazine-1-carboxylate (382 mg, crude) which was used in the next reaction without further purification. LCMS (ESI) m/z: [M+H] calcd for C₆₁H₇₇N₉O₉: 1080.6; found 1080.6.

Step 2. To a solution of benzyl 4-(5-((3aS,6S,9S,15S,32aS)-22-ethyl-6-isopropyl-5,19,19-trimethyl-4,7,10,16-tetraoxo-2,3,3a,4,5,6,7,8,9,10,13,14,15,16,18,19,20,22,32,32a-icosahydro-1H,12H-11,15-epimino-23,25-etheno-9,28-methano-26,30-(metheno)dipyrrolo[2,3-c:3′,4′-v][1,18]dioxa[6,9,12]triazacyclotriacontin-21-yl)-6-((S)-1-methoxyethyl)pyridin-3-yl)piperazine-1-carboxylate (380 mg, crude) in DMF (4.0 mL) stirred at 0° C. were added DIPEA (455 mg, 3.52 mmol), 2-fluoro-2-methylpropanoic acid (74.6 mg, 0.704 mmol), and COMU (301 mg, 0.704 mmol). The resulting mixture was stirred for 3 h at room temperature and was then quenched at 0° C. by the addition H₂O. The aqueous mixture was extracted with EtOAc (3×10 mL), and the combined organic extracts were washed with brine (10 mL), dried over Na₂SO₄, filtered, and concentrated under reduced pressure. Purification by normal phase chromatography afforded benzyl 4-(5-((3aS,6S,9S,15S,32aS)-22-ethyl-1-(2-fluoro-2-methylpropanoyl)-6-isopropyl-5,19,19-trimethyl-4,7,10,16-tetraoxo-2,3,3a,4,5,6,7,8,9,10,13,14,15,16,18,19,20,22,32,32a-icosahydro-1H,12H-11,15-epimino-23,25-etheno-9,28-methano-26,30-(metheno)dipyrrolo[2,3-c:3′,4′-v][1,18]dioxa[6,9,12]triazacyclotriacontin-21-yl)-6-((S)-1-methoxyethyl)pyridin-3-yl)piperazine-1-carboxylate (422 mg, crude), which was used in the next reaction without further purification. LCMS (ESI) m/z: [M+H] calcd for C₆₅H₈₂FN₉O₁₀: 1168.6; found 1169.1.

Step 3. To a solution of benzyl 4-(5-((3aS,6S,9S,15S,32aS)-22-ethyl-1-(2-fluoro-2-methylpropanoyl)-6-isopropyl-5,19,19-tri methyl-4,7,10,16-tetraoxo-2,3,3a,4,5,6,7,8,9,10,13,14,15,16,18,19,20,22,32,32a-icosahydro-1H,12H-11,15-epimino-23,25-etheno-9,28-methano-26,30-(metheno)dipyrrolo[2,3-c:3′,4′-v][1,18]dioxa[6,9,12]triazacyclotriacontin-21-yl)-6-((S)-1-methoxyethyl)pyridin-3-yl)piperazine-1-carboxylate (420 mg, crude) in MeOH (5.0 mL) was added Pd(OH)₂/C (210 mg). The resulting mixture was stirred for 2 h at room temperature under an atmosphere of H₂ and filtered. The filter cake was washed with MeOH (4×30 mL) and the filtrate concentrated under reduced pressure. Purification by reversed phase chromatography afforded (3aS,6S,9S,15S,32aS)-22-ethyl-1-(2-fluoro-2-methylpropanoyl)-6-isopropyl-21-(2-((S)-1-methoxyethyl)-5-(piperazin-1-yl)pyridin-3-yl)-5,19,19-trimethyl-3,3a,5,6,8,9,14,15,18,19,20,22,32,32a-tetradecahydro-1H,12H-11,15-epimino-23,25-etheno-9,28-methano-26,30-(metheno)dipyrrolo[2,3-c:3′,4′-v][1,18]dioxa[6,9,12]triazacyclotriacontine-4,7,10,16(2H,13H)-tetraone (76.1 mg, 22% yield over 3 steps) as a light orange solid. LCMS (ESI) m/z: [M+H] calcd for C₅₇H₇₆FN₉O₈: 1034.6; found 1034.6; ¹H NMR (400 MHz, DMSO-d₆) δ 8.38 (m, 1H), 8.01 (m, 1H), 7.72-7.57 (m, 1H), 7.50 (t, J=7.9 Hz, 1H), 7.40 (d, J=9.9 Hz, 1H), 7.31 (s, 1H), 7.21 (s, 1H), 7.18-7.02 (m, 1H), 6.76-6.53 (m, 1H), 5.36-4.91 (m, 1H), 4.62 (d, J=10.7 Hz, 1H), 4.58-3.88 (m, 7H), 3.85 (s, 1H), 3.70 (s, 4H), 3.10 (s, 2H), 2.96 (s, 5H), 2.72 (m, 3H), 2.56 (s, 7H), 2.48 (s, 1H), 2.33 (s, 1H), 2.19 (d, J=15.9 Hz, 1H), 2.10-1.68 (m, 3H), 1.72-1.50 (m, 4H), 1.48 (d, J=4.6 Hz, 2H), 1.42 (s, 2H), 1.30 (d, J=6.2 Hz, 3H), 1.26-1.04 (m, 4H), 0.94 (m, 2H), 0.87 (s, 1H), 0.84-0.58 (m, 8H), 0.47 (m, 3H).

### Example A424. Synthesis of (4aR,7S,10S,16S)-1-acetyl-22-(5-(4-cyclopropylpiperazin-1-yl)-2-((S)-1-methoxyethyl)pyridin-3-yl)-23-ethyl-7-isopropyl-6,20,20-trimethyl-2,3,4,4a,6,7,9,10,15,16,19,20,21,23,32,33-hexadecahydro-13H-12,16-epimino-24,26-etheno-10,29-methano-27,31-(metheno)pyridazino[6,1-o]pyrrolo[3,4-z][1]oxa[7,10,13,16]tetraazacyclotriacontine-5,8,11,17(1H,14H)-tetraone

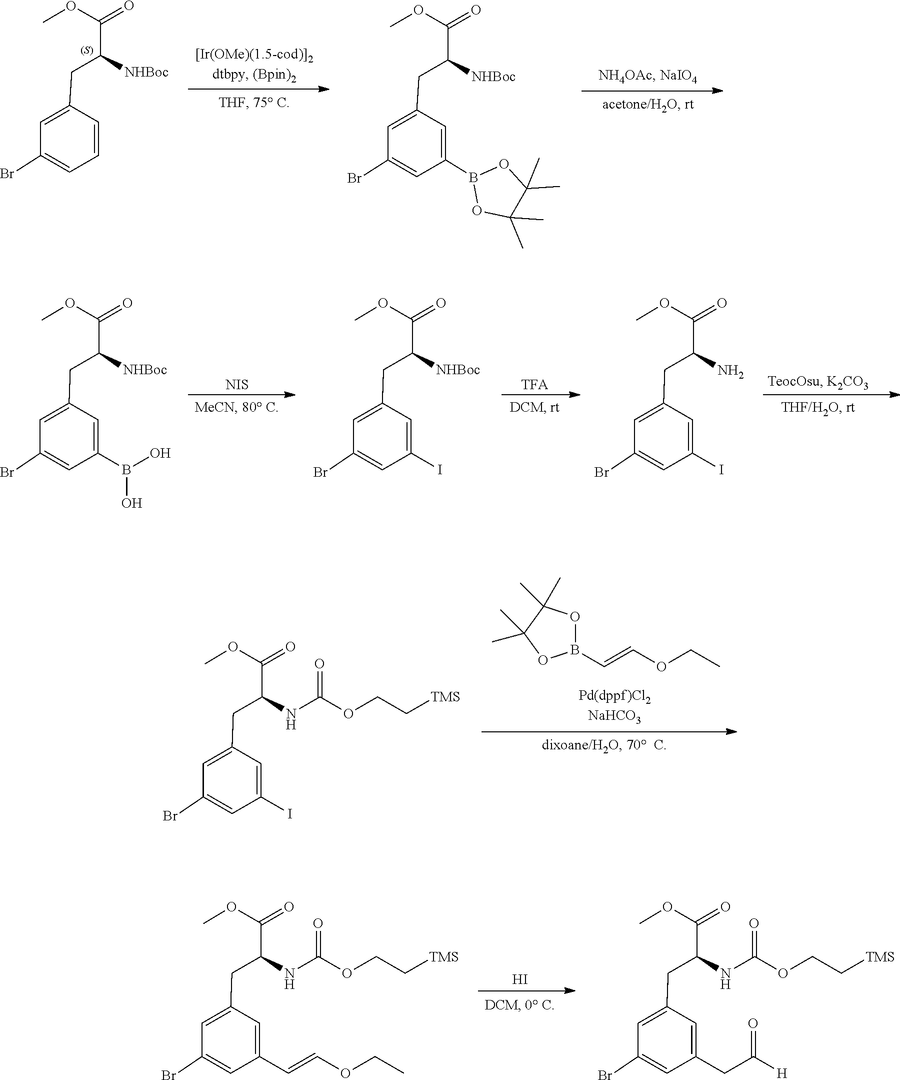

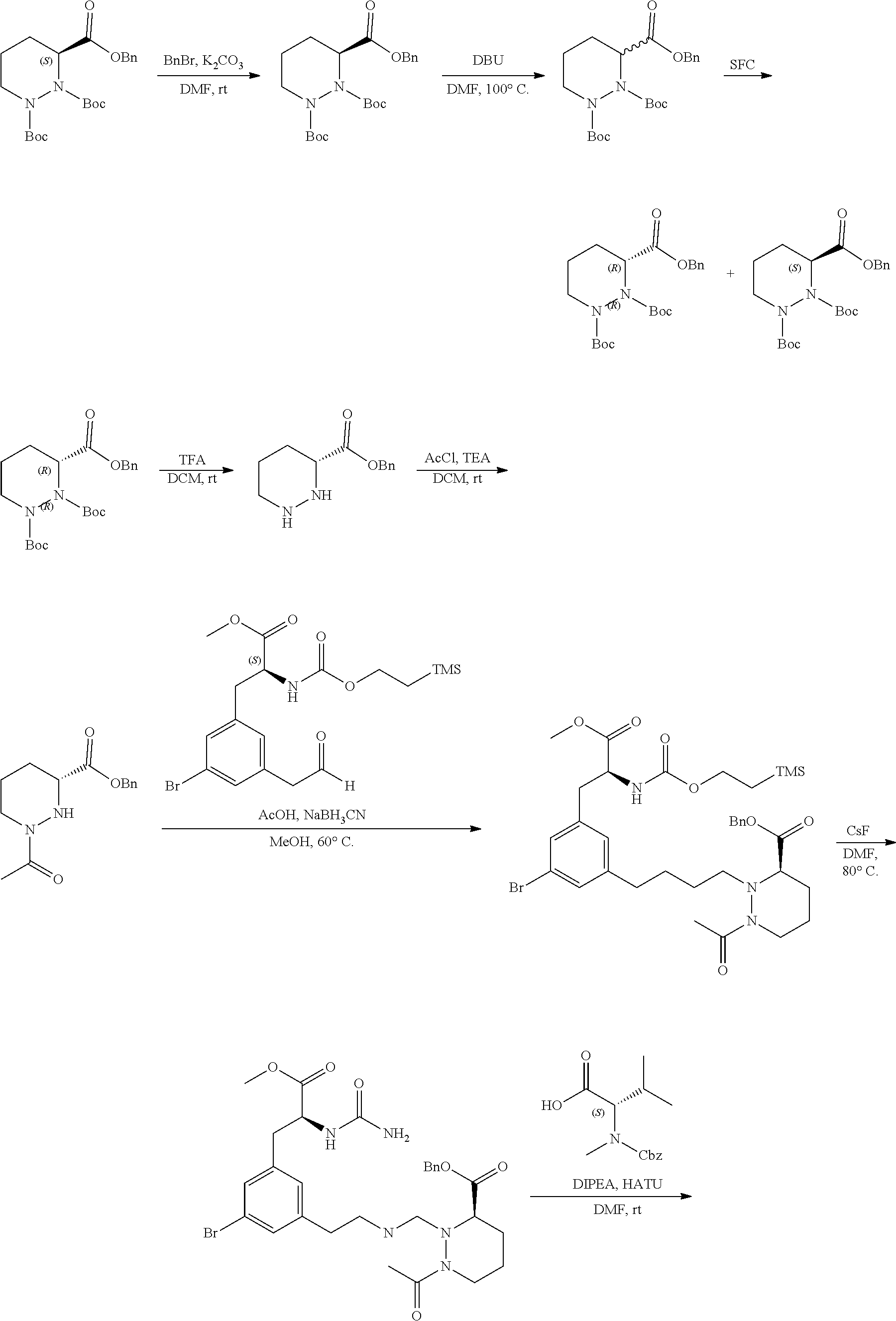

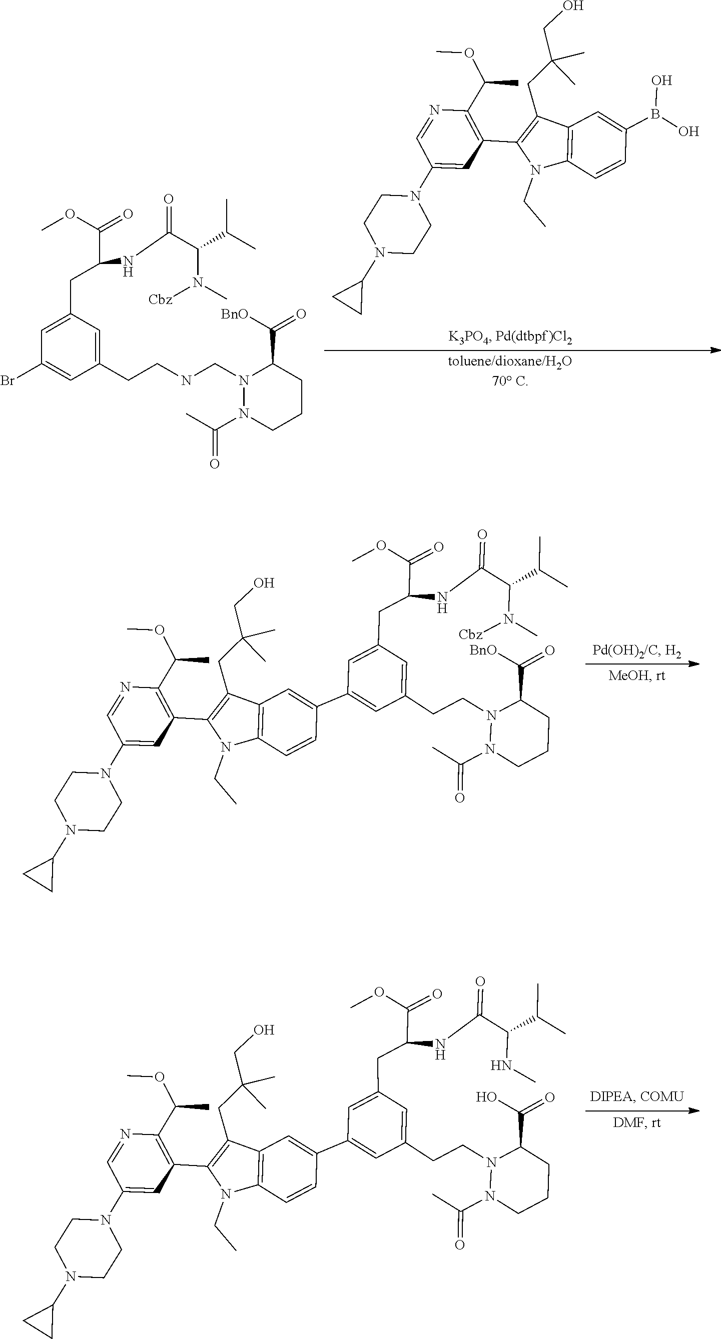

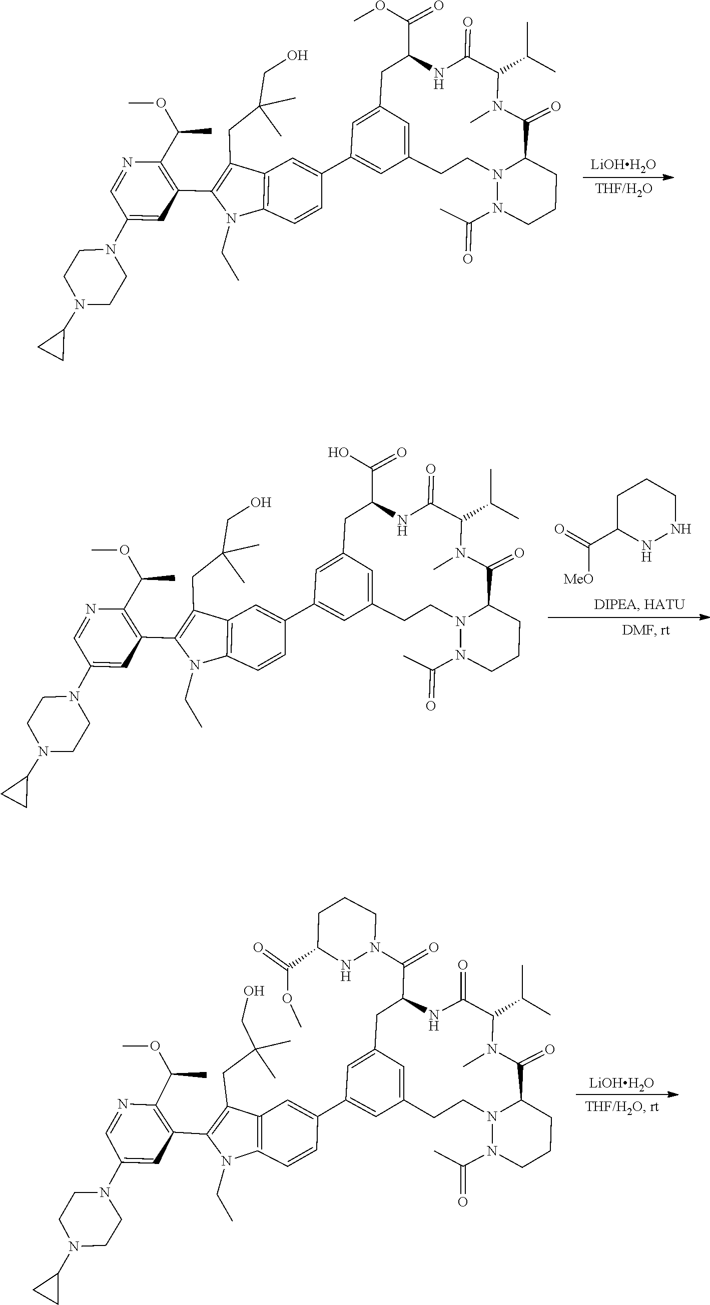

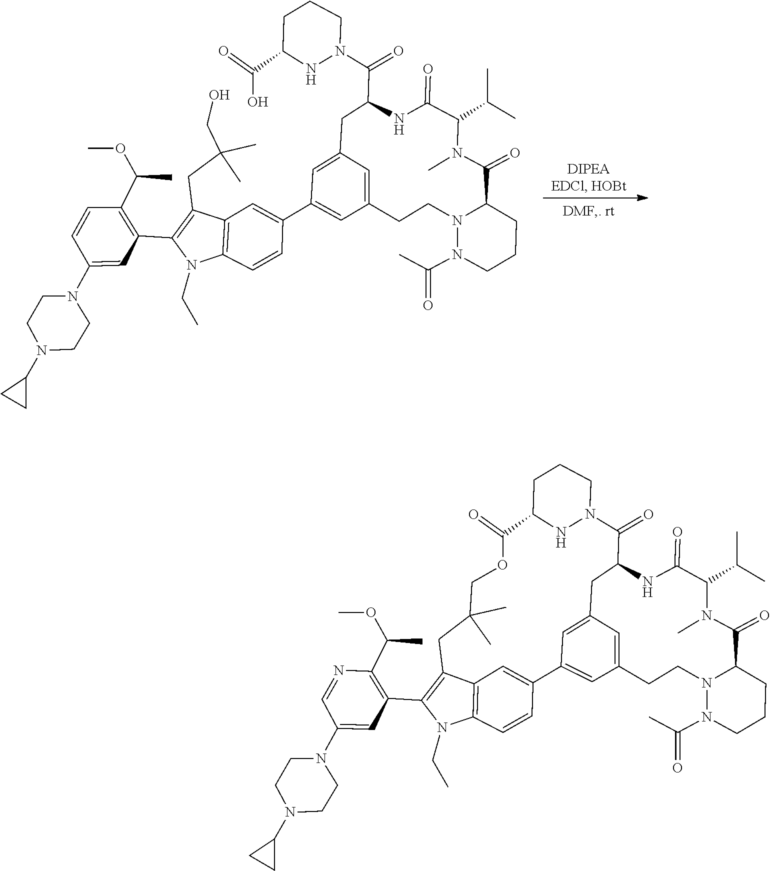

Step 1. To a stirred solution of methyl (S)-3-(3-bromophenyl)-2-((tert-butoxycarbonyl)amino)propanoate (50.0 g, 140 mmol) and dtbpy (5.62 g, 20.9 mmol) in THF (200 mL) were added [Ir(OMe)(1,5-cod)]2 (2.78 g, 4.19 mmol) and methyl 4-(4,4,5,5-tetramethyl-1,3,2-dioxaborolan-2-yl)pyridine-2-carboxylate (36.7 g, 140 mmol) at room temperature. The resulting mixture was stirred overnight at 75° C. and the reaction was quenched by the addition of H₂O at 0° C. The aqueous mixture was extracted with EtOAc (3×500 mL), dried over Na₂SO₄, filtered, and concentrated under reduced pressure to give methyl (S)-3-(3-bromo-5-(4,4,5,5-tetramethyl-1,3,2-dioxaborolan-2-yl)phenyl)-2-((tert-butoxycarbonyl)amino)propanoate (70.0 g, crude), which was used without further purification. LCMS (ESI) m/z: [M−C₄H₈+H] calcd for C₂₁H₃₁BBrNO₆: 427.1; found 427.1.

Step 2. To a mixture of methyl (S)-3-(3-bromo-5-(4,4,5,5-tetramethyl-1,3,2-dioxaborolan-2-yl)phenyl)-2-((tert-butoxycarbonyl)amino)propanoate (70.0 g, crude) and NaIO₄ (155 g, 723 mmol) in acetone (500 mL) was added a solution of NH₄OAc (55.7 g, 723 mmol) in H₂O (250 mL) at room temperature. The resulting mixture was stirred for 12 h at room temperature, filtered, and the filter cake washed with EtOAc (3×300 mL). The filtrate was concentrated under reduced pressure, extracted with EtOAc (3×500 mL), dried over Na₂SO₄, filtered, and concentrated under reduced pressure to give (S)-(3-bromo-5-(2-((tert-butoxycarbonyl)amino)-3-methoxy-3-oxopropyl)phenyl)boronic acid (72.0 g, crude), which was used without further purification. LCMS (ESI) m/z: [M+Na] calcd for C₂₁H₂₁BBrNO₆: 424.1; found 424.1.

Step 3. To a stirred solution of (S)-(3-bromo-5-(2-((tert-butoxycarbonyl)amino)-3-methoxy-3-oxopropyl)phenyl)boronic acid (72.0 g, crude) in MeCN (750 mL) was added NIS (168 g, 746 mmol) at room temperature. The resulting mixture was stirred overnight at 80° C., filtered, and the filter cake washed with EtOAc (3×100 mL). The filtrate was then concentrated under reduced pressure and purified by normal phase chromatography, to afford methyl (S)-3-(3-bromo-5-iodophenyl)-2-((tert-butoxycarbonyl)amino)propanoate (50.0 g, 74% yield over 3 steps) as a yellow solid. LCMS (ESI) m/z: [M+H] calcd for C₁₅H₁₉BrINO₄: 483.9; found 483.9.

Step 4. To a solution of methyl (S)-3-(3-bromo-5-iodophenyl)-2-((tert-butoxycarbonyl)amino)propanoate (50.0 g, 103 mmol) in DCM (300 mL) was added TFA (100 mL) at 0° C. The resulting mixture was stirred overnight at 0° C. and was then acidified to pH=8 by the addition of sat. aq. NaHCO₃. The resulting mixture was extracted with DCM (3×500 mL), dried over Na₂SO₄, filtered, and concentrated under reduced pressure to afford methyl (S)-2-amino-3-(3-bromo-5-iodophenyl)propanoate (37.0 g, crude), which was used without further purification. LCMS (ESI) m/z: [M+H] calcd for C₁₀H₁₁BrINO₂: 383.9; found 383.8.

Step 5. To a mixture of methyl (S)-2-amino-3-(3-bromo-5-iodophenyl)propanoate (33.0 g, crude), and K₂CO₃ (35.0 g, 258 mmol) in THE (120 mL) and H₂O (120 mL) was added 2,5-dioxopyrrolidin-1-yl 2,2,2-trichloroethyl carbonate (37.4 g, 129 mmol) at 0° C. The resulting mixture was stirred for 2 h at room temperature and was then extracted with EtOAc (3×300 mL). The combined organic extracts were dried over Na₂SO₄, filtered, and concentrated under reduced pressure. The residue was purified by normal phase chromatography to afford methyl (S)-3-(3-bromo-5-iodophenyl)-2-(((2-(trimethylsilyl)ethoxy)carbonyl)amino)propanoate (43.0 g, 84% yield over 2 steps) as a yellow oil. LCMS (ESI) m/z: [M+H] calcd for C₁₆H₂₃BrINO₄Si: 528.0 found 528.0.

Step 6. A mixture of methyl (S)-3-(3-bromo-5-iodophenyl)-2-(((2(trimethylsilyl)ethoxy)carbonyl)amino)propanoate (47.0 g, 89.0 mmol), (E)-2-(2-ethoxyvinyl)-4,4,5,5-tetramethyl-1,3,2-dioxaborolane (19.4 g, 97.9 mmol), NaHCO₃ (22.4 g, 267 mmol), and Pd(dppf)Cl₂ (3.91 g, 5.34 mmol) in dioxane (450 mL) and H₂O (150 mL) was stirred for 3 h at 70° C. The reaction mixture was then quenched by the addition of H₂O at 0° C., extracted with EtOAc (3×500 mL), dried over Na₂SO₄, filtered, and concentrated under reduced pressure. The crude residue was purified by reversed phase chromatography to afford methyl (S,E)-3-(3-bromo-5-(2-ethoxyvinyl)phenyl)-2-(((2-(trimethylsilyl)ethoxy)carbonyl)amino)propanoate (34.7 g, 72% yield) as a brown oil. LCMS (ESI) m/z: [M−C₂H₄+H] calcd for C₂₀H₃₀BrNO₅Si: 444.1; found 444.1.

Step 7. To a solution of methyl (S,E)-3-(3-bromo-5-(2-ethoxyvinyl)phenyl)-2-(((2-(trimethylsilyl)ethoxy)carbonyl)amino)propanoate (10.0 g, 2.12 mmol) in DCM (500 mL) was added HI (50 mL) dropwise at 0° C. The resulting mixture was stirred for 1 h at 0° C. and was then basified to pH 8 with by the addition of sat. aq. NaHCO₃. The aqueous phase was extracted with DCM (3×500 mL), dried over Na₂SO₄, filtered, and concentrated under reduced pressure to give methyl (S)-3-(3-bromo-5-(2-oxoethyl)phenyl)-2-(((2-(trimethylsilyl)ethoxy)carbonyl)amino)propanoate (8.10 g, crude), which was used without further purification. LCMS (ESI) m/z: [M+H] calcd for C₁₈H₂₆BrNO₅Si: 444.1 found 444.1.

Step 8. To a solution of (S)-1,2-bis(tert-butoxycarbonyl)hexahydropyridazine-3-carboxylic acid (50.0 g, 151 mmol) and K₂CO₃ (62.8 g, 454 mmol) in DMF (500 mL) was added benzyl bromide (27.0 mL, 227 mmol) at 0° C. The resulting mixture was stirred for 2 h at room temperature and was then quenched by the addition of H₂O (600 mL) at. The aqueous mixture was extracted with EtOAc (3×500 mL) and the combined organic extracts were washed with brine (3×1.5 L), dried over Na₂SO₄, filtered, and concentrated under reduced pressure. The residue was purified by normal phase chromatography to afford 3-benzyl 1,2-di-tert-butyl (S)-tetrahydropyridazine-1,2,3-tricarboxylate (60.0 g, 94% yield) as a yellow solid. LCMS (ESI) m/z: [M+Na] calcd for C₂₂H₃₂N₂O₆: 443.2; found 443.2.

Step 9. A solution 3-benzyl 1,2-di-tert-butyl (S)-tetrahydropyridazine-1,2,3-tricarboxylate (60.0 g, 157 mmol) and DBU (117 mL, 786 mmol) in DMF (660 mL) was stirred for 12 h at 100° C. under an atmosphere of N₂. The resulting mixture was quenched by the addition of H₂O (600 mL), extracted with EtOAc (3×500 mL), treated with brine (3×1 L), dried over Na₂SO₄, filtered, and concentrated under reduced pressure. The residue was purified by normal phase chromatography to afford 3-benzyl 1,2-di-tert-butyl 1,2-diazinane-1,2,3-tricarboxylate (45.4 g, 69% yield) as a yellow oil. LCMS (ESI) m/z: [M+Na] calcd for C₂₂H₃₂N₂O₆: 443.2; found 443.2.

Step 10. Methyl 3-benzyl 1,2-di-tert-butyl 1,2-diazinane-1,2,3-tricarboxylate (45.4 g) was purified by prep-SFC to give the faster eluting 3-benzyl 1,2-di-tert-butyl (R)-tetrahydropyridazine-1,2,3-tricarboxylate (23.3 g, 51% yield) as a white solid and the slower eluting 3-benzyl 1,2-di-tert-butyl (S)-tetrahydropyridazine-1,2,3-tricarboxylate (22.1 g, 49% yield) as a white solid. LCMS (ESI) m/z: [M+Na] calcd for C₂₂H₃₂N₂O₆: 443.2; found 443.2.

Step 11. To a solution of 3-benzyl 1,2-di-tert-butyl (R)-tetrahydropyridazine-1,2,3-tricarboxylate (11.0 g, 26.2 mmol) in DCM (100 mL) was added TFA (50 mL) at 0° C. The resulting mixture was stirred for 2 h at rt and was then concentrated under reduced pressure to afford benzyl (R)-hexahydropyridazine-3-carboxylate (12.9 g, crude), which was used without further purification. LCMS (ESI) m/z: [M+H] calcd for C₁₂H₁₆N₂O₂: 221.1; found 221.1.

Step 12. To a stirred solution of benzyl (R)-hexahydropyridazine-3-carboxylate (12.4 g, crude) and TEA (15.0 g, 148 mmol) in DCM (120 mL) was added AcCl (2.09 g, 26.7 mmol) at 0° C. The resulting mixture was stirred for 2 h at rt and was then quenched by the addition of H₂O at 0° C. The aqueous phase was extracted with DCM (3×100 mL) and the combined organic extracts were washed with H₂O (3×300 mL), dried over Na₂SO₄, filtered, and concentrated under reduced pressure. The residue was purified by normal phase chromatography to afford benzyl (R)-1-acetylhexahydropyridazine-3-carboxylate (6.20 g, 94% yield over 2 step) as a yellow oil. LCMS (ESI) m/z: [M+H] calcd for C₁₄H₁₈N₂O₃: 263.1; found 263.2.

Step 13. To a solution of benzyl (R)-1-acetylhexahydropyridazine-3-carboxylate (800 mg, 3.05 mmol) in MeOH (2 mL) were added AcOH (1.10 g, 18.3 mmol) and methyl (S)-3-(3-bromo-5-(2-oxoethyl)phenyl)-2-(((2-(trimethylsilyl)ethoxy)carbonyl)amino)propanoate (8.13 g, crude). The resulting mixture was stirred for 40 min at room temperature, after which time NaBH₃CN (3.83 g, 61.0 mmol) was added at 0° C. The reaction mixture was stirred overnight at 60° C. and was the concentrated under reduced pressure. The residue was dissolved in DCM (50 mL), treated with sat. aq. NaHCO₃ (3×100 mL), concentrated under reduced pressure and purified by reversed phase chromatography to afford benzyl (R)-1-acetyl-2-(3-bromo-5-((S)-3-methoxy-3-oxo-2-(((2-(trimethylsilyl)ethoxy)carbonyl)amino)propyl)phenethyl)hexahydropyridazine-3-carboxylate (219 mg, 9.3% yield) as a yellow oil. LCMS (ESI) m/z: [M+H] calcd for C₃₂H₄₄BrN₃₀₇Si: 692.2; found 692.2.

Step 14. To a solution of benzyl (R)-1-acetyl-2-(3-bromo-5-((S)-3-methoxy-3-oxo-2-(((2-(trimethylsilyl)ethoxy)carbonyl)amino)propyl)phenethyl)hexahydropyridazine-3-carboxylate (340 mg, 0.492 mmol) in DMF (4 mL) was added CsF (374 mg, 2.46 mmol). The resulting mixture was stirred for 3 h at 80° C. and was then quenched by the addition of H₂O at 0° C. The aqueous mixture was extracted with EtOAc (3×10 mL) and the combined organic extracts were treated with brine (3×10 mL), dried over Na₂SO₄, filtered, and concentrated under reduced pressure to afford benzyl (R)-1-acetyl-2-(3-((S)-2-amino-3-methoxy-3-oxopropyl)-5-bromophenethyl)hexahydropyridazine-3-carboxylate (280 mg, crude), which was used without further purification. LCMS (ESI) m/z: [M+H] calcd for C₂₆H₃₂BrN₃O₅: 548.2; found 548.1.

Step 15. To a stirred solution of benzyl (R)-1-acetyl-2-(3-((S)-2-amino-3-methoxy-3-oxopropyl)-5-bromophenethyl)hexahydropyridazine-3-carboxylate (280 mg, crude), DIPEA (662 mg, 5.12 mmol) and N-((benzyloxy)carbonyl)-N-methyl-L-valine (272 mg, 1.02 mmol) in DMF (4 mL) was added HATU (292 mg, 0.768 mmol) at 0° C. The resulting mixture was stirred for 1 h at room temperature and was then quenched by the addition of H₂O at 0° C. The aqueous mixture was extracted with EtOAc (3×10 mL), treated with brine (3×10 mL), dried over Na₂SO₄, filtered, and concentrated under reduced pressure. The residue was purified by normal phase chromatography to afford benzyl (R)-1-acetyl-2-(3-((S)-2-((S)-2-(((benzyloxy)carbonyl)(methyl)amino)-3-methylbutanamido)-3-methoxy-3-oxopropyl)-5-bromophenethyl)hexahydropyridazine-3-carboxylate (270 mg, 69% yield over 2 steps) as a yellow oil. LCMS (ESI) m/z: [M+H] calcd for C₄₀H₄₉BrN₄O₈: 795.3; found 795.3.

Step 16. To a stirred solution of benzyl (R)-1-acetyl-2-(3-((S)-2-((S)-2-(((benzyloxy)carbonyl)(methyl)amino)-3-methylbutanamido)-3-methoxy-3-oxopropyl)-5-bromophenethyl)hexahydropyridazine-3-carboxylate (270 mg, 0.340 mmol) and (S)-(2-(5-(4-cyclopropylpiperazin-1-yl)-2-(1-methoxyethyl)pyridin-3-yl)-1-ethyl-3-(3-hydroxy-2,2-dimethylpropyl)-1H-indol-5-yl)boronic acid (273 mg, 0.510 mmol) in dioxane (0.5 mL) and H₂O (0.5 mL) were added K₃PO₄ (144 mg, 0.680 mmol) and a solution of Pd(dtbpfCl2 (22.2 mg, 0.034 mmol) in toluene (1.5 mL) at room temperature. The resulting mixture was stirred for 3 h at 70° C. under an atmosphere of N₂ and was then quenched by the addition of H₂O at 0° C. The aqueous mixture was extracted with EtOAc (3×10 mL), dried over Na₂SO₄, filtered, and concentrated under reduced pressure. The residue was purified by prep-TLC to afford benzyl (R)-1-acetyl-2-(3-((S)-2-((S)-2-(((benzyloxy)carbonyl)(methyl)amino)-3-methylbutanamido)-3-methoxy-3-oxopropyl)-5-(2-(5-(4-cyclopropylpiperazin-1-yl)-2-((S)-1-methoxyethyl)pyridin-3-yl)-1-ethyl-3-(3-hydroxy-2,2-dimethylpropyl)-1H-indol-5-yl)phenethyl)hexahydropyridazine-3-carboxylate (245 mg, 54% yield) as a brown solid. LCMS (ESI) m/z: [M+H] calcd for C₇₀H₉₀N₈O₁₀: 1203.7; found 1203.7.

Step 17. To a stirred solution of benzyl (R)-1-acetyl-2-(3-((S)-2-((S)-2-(((benzyloxy)carbonyl)(methyl)amino)-3-methylbutanamido)-3-methoxy-3-oxopropyl)-5-(2-(5-(4-cyclopropylpiperazin-1-yl)-2-((S)-1-methoxyethyl)pyridin-3-yl)-1-ethyl-3-(3-hydroxy-2,2-dimethylpropyl)-1H-indol-5-yl)phenethyl)hexahydropyridazine-3-carboxylate (180 mg, 0.150 mmol) in MeOH (2 mL) was added Pd(OH)₂/C (180 mg) at room temperature under an atmosphere of N₂. The resulting mixture was stirred for 1 h at room temperature under an atmosphere of H₂, filtered, the filter cake washed with MeOH (3×10 mL) and the filtrate concentrated under reduced pressure to afford (R)-1-acetyl-2-(3-(2-(5-(4-cyclopropylpiperazin-1-yl)-2-((S)-1-methoxyethyl)pyridin-3-yl)-1-ethyl-3-(3-hydroxy-2,2-dimethylpropyl)-1H-indol-5-yl)-5-((S)-3-methoxy-2-((S)-3-methyl-2-(methylamino)butanamido)-3-oxopropyl)phenethyl)hexahydropyridazine-3-carboxylic acid (130 mg, crude), which was used without further purification. LCMS (ESI) m/z: [M/2+H] calcd for C₅₅H₇₈N₈O₄: 490.3; found 490.5.

Step 18. To a stirred solution of (R)-1-acetyl-2-(3-(2-(5-(4-cyclopropylpiperazin-1-yl)-2-((S)-1-methoxyethyl)pyridin-3-yl)-1-ethyl-3-(3-hydroxy-2,2-dimethylpropyl)-1H-indol-5-yl)-5-((S)-3-methoxy-2-((S)-3-methyl-2-(methylamino)butanamido)-3-oxopropyl)phenethyl)hexahydropyridazine-3-carboxylic acid (160 mg, crude) and DIPEA (211 mg, 1.63 mmol) in DMF (3 mL) was added COMU (140 mg, 0.326 mmol) at 0° C. The resulting mixture was stirred for 1 h at room temperature and was quenched by the addition of H₂O at 0° C. The aqueous mixture was extracted with EtOAc (3×10 mL) and the combined organic extracts were washed with brine (3×10 mL), dried over Na₂SO₄, filtered, and concentrated under reduced pressure. The residue was purified by reversed phase chromatography to afford methyl (1⁶R,6S,9S)-1²-acetyl-4⁵-(2-(5-(4-cyclopropylpiperazin-1-yl)-2-((S)-1-methoxyethyl)pyridin-3-yl)-1-ethyl-3-(3-hydroxy-2,2-dimethylpropyl)-1H-indol-5-yl)-9-isopropyl-10-methyl-8,11-dioxo-1¹,1²,1³,1⁴,1⁵,1⁶-hexahydro-7,10-diaza-1(1,6)-pyridazina-4(1,3)-benzenacycloundecaphane-6-carboxylate (75 mg, 42% yield over 2 steps) as a yellow solid. LCMS (ESI) m/z: [M+H] calcd for C₅₅H₇₆N₈O₇: 961.6; found 961.4.

Step 19. To a stirred solution of methyl (1⁶R,6S,9S)-1²-acetyl-4⁵-(2-(5-(4-cyclopropylpiperazin-1-yl)-2-((S)-1-methoxyethyl)pyridin-3-yl)-1-ethyl-3-(3-hydroxy-2,2-dimethylpropyl)-1H-indol-5-yl)-9-isopropyl-10-methyl-8,11-dioxo-1¹,1²,1³,1⁴,1⁵,1⁶-hexahydro-7,10-diaza-1(1,6)-pyridazina-4(1,3)-benzenacycloundecaphane-6-carboxylate (40 mg, 0.042 mmol) and H₂O (1 mL) in THE (1 mL) was added LiOH·H₂O (3.49 mg, 0.084 mmol) at 0° C. The resulting mixture was stirred for 2 h at room temperature and was then acidified to pH 6 with 1 N aq. HCl. The aqueous mixture was extracted with 3:1 vol. DCM:IPA (3×10 mL), dried over Na₂SO₄, filtered, and concentrated under reduced pressure to afford (1⁶R,6S,9S)-1²-acetyl-4⁵-(2-(5-(4-cyclopropylpiperazin-1-yl)-2-((S)-1-methoxyethyl)pyridin-3-yl)-1-ethyl-3-(3-hydroxy-2,2-dimethylpropyl)-1H-indol-5-yl)-9-isopropyl-10-methyl-8,11-dioxo-1¹,1²,1³,1⁴,1⁵,1⁶-hexahydro-7,10-diaza-1 (1,6)-pyridazina-4(1,3)-benzenacycloundecaphane-6-carboxylic acid (68 mg, crude), which was used without further purification. LCMS (ESI) m/z: [M+H] calcd for C₅₄H₇₄N₈O₇: 947.6; found 947.5.

Step 20. To a stirred solution of (1⁶R,6S,9S)-1²-acetyl-4⁵-(2-(5-(4-cyclopropylpiperazin-1-yl)-2-((S)-1-methoxyethyl)pyridin-3-yl)-1-ethyl-3-(3-hydroxy-2,2-dimethylpropyl)-1H-indol-5-yl)-9-isopropyl-10-methyl-8,11-dioxo-1¹,1²,1³,1⁴,1⁵,1⁶-hexahydro-7,10-diaza-1(1,6)-pyridazina-4(1,3)-benzenacycloundecaphane-6-carboxylic acid (58 mg, crude), DIPEA (79.1 mg, 0.610 mmol) and methyl (3S)-1,2-diazinane-3-carboxylate (17.7 mg, 0.122 mmol) in DMF (1 mL) was added HATU (46.6 mg, 0.122 mmol) at 0° C. The resulting mixture was stirred for 1 h at room temperature and was then quenched by the addition of H₂O at 0° C. The aqueous mixture was extracted with EtOAc (3×10 mL) and the combined organic extracts were washed with brine (3×10 mL), dried over Na₂SO₄, filtered, and concentrated under reduced pressure to afford methyl (S)-1-((1⁶R,6S,9S)-1²-acetyl-4⁵-(2-(5-(4-cyclopropylpiperazin-1-yl)-2-((S)-1-methoxyethyl)pyridin-3-yl)-1-ethyl-3-(3-hydroxy-2,2-dimethylpropyl)-1H-indol-5-yl)-9-isopropyl-10-methyl-8,11-dioxo-1¹,1²,1³,1⁴,1⁵,1⁶-hexahydro-7,10-diaza-1(1,6)-pyridazina-4(1,3)-benzenacycloundecaphane-6-carbonyl)hexahydropyridazine-3-carboxylate (92.4 mg, crude), which was used without further purification. LCMS (ESI) m/z: [M+H] calcd for C₆₀H₈₄N₁₀O₈: 1073.7; found 1073.5.

Step 21. To a stirred solution of methyl (S)-1-((1⁶R,6S,9S)-1²-acetyl-4⁵-(2-(5-(4-cyclopropylpiperazin-1-yl)-2-((S)-1-methoxyethyl)pyridin-3-yl)-1-ethyl-3-(3-hydroxy-2,2-dimethylpropyl)-1H-indol-5-yl)-9-isopropyl-10-methyl-8,11-dioxo-1¹,1²,1³,1⁴,1⁵,1⁶-hexahydro-7,10-diaza-1(1,6)-pyridazina-4(1,3)-benzenacycloundecaphane-6-carbonyl)hexahydropyridazine-3-carboxylate (90.0 mg, crude) in H₂O (1 mL) and THE (1 mL) was added LiOH·H₂O (7.04 mg, 0.168 mmol) at 0° C. The resulting mixture was stirred for 2 h at room temperature and was then acidified to pH 6 by the addition of 1 N aq. HCl. The aqueous mixture was extracted with 3:1 vol. DCM:IPA (3×10 mL), dried over Na₂SO₄, filtered, and concentrated under reduced pressure to afford (S)-1-((1⁶R,6S,9S)-1²-acetyl-4⁵-(2-(5-(4-cyclopropylpiperazin-1-yl)-2-((S)-1-methoxyethyl)pyridin-3-yl)-1-ethyl-3-(3-hydroxy-2,2-dimethylpropyl)-1H-indol-5-yl)-9-isopropyl-10-methyl-8,11-dioxo-1¹,1²,1³,1⁴,1⁵,1⁶-hexahydro-7,10-diaza-1(1,6)-pyridazina-4(1,3)-benzenacycloundecaphane-6-carbonyl)hexahydropyridazine-3-carboxylic acid (90 mg, crude), which was used without further purification. LCMS (ESI) m/z: [M+H] calcd for C₅₉H₈₂N₁₀O₈: 1059.6; found 1059.5.

Step 22. To a stirred solution of (S)-1-((1⁶R,6S,9S)-1²-acetyl-4⁵-(2-(5-(4-cyclopropylpiperazin-1-yl)-2-((S)-1-methoxyethyl)pyridin-3-yl)-1-ethyl-3-(3-hydroxy-2,2-dimethylpropyl)-1H-indol-5-yl)-9-isopropyl-10-methyl-8,11-dioxo-1¹,1²,1³,1⁴,1⁵,1⁶-hexahydro-7,10-diaza-1(1,6)-pyridazina-4(1,3)-benzenacycloundecaphane-6-carbonyl)hexahydropyridazine-3-carboxylic acid (90 mg, crude) and DIPEA (329 mg, 2.55 mmol) in DCM (9 mL) were added EDCI (326 mg, 1.70 mmol) and HOBt (115 mg, 0.850 mmol) at 0° C. The resulting mixture was stirred for overnight at room temperature and was then acidified to pH 6 by the addition of 1 N aq. HCl. The aqueous phase was extracted with DCM (3×20 mL), dried over Na₂SO₄, filtered, and concentrated under reduced pressure. The residue was purified by reversed phase chromatography to afford (4aR,7S,10S,16S)-1-acetyl-22-(5-(4-cyclopropylpiperazin-1-yl)-2-((S)-1-methoxyethyl)pyridin-3-yl)-23-ethyl-7-isopropyl-6,20,20-trimethyl-2,3,4,4a,6,7,9,10,15,16,19,20,21,23,32,33-hexadecahydro-13H-12,16-epimino-24,26-etheno-10,29-methano-27,31-(metheno)pyridazino[6,1-o]pyrrolo[3,4-z][1]oxa[7,10,13,16]tetraazacyclotriacontine-5,8,11,17(1H,14H)-tetraone (5.7 mg, 16% yield over 4 steps) as a white solid. LCMS (ESI) m/z: [M+H] calcd for C₅₉H₈₀N₁₀O₇: 1041.6; found 1041.6. ¹H NMR (400 MHz, DMSO-d₆) δ 8.52 (d, J=8.2 Hz, 1H), 8.39 (d, J=2.8 Hz, 1H), 7.93 (s, 1H), 7.61 (d, J=8.7 Hz, 1H), 7.57-7.44 (m, 2H), 7.37 (s, 1H), 7.17 (d, J=2.8 Hz, 1H), 7.06 (s, 1H), 5.30 (d, J=12.3 Hz, 1H), 5.23-5.14 (m, J=9.7 Hz, 1H), 4.68 (d, J=10.8 Hz, 1H), 4.33-4.12 (m, 3H), 4.10-3.87 (m, 4H), 3.59 (s, 2H), 3.51-3.41 (m, 1H), 3.21-3.09 (m, 4H), 3.05-2.98 (m, 3H), 2.83 (d, J=12.9 Hz, 2H), 2.68 (d, J=12.2 Hz, 3H), 2.61 (d, J=5.2 Hz, 7H), 2.35 (d, J=13.7 Hz, 1H), 2.14-2.08 (m, 1H), 1.91 (d, J=12.4 Hz, 5H), 1.77 (d, J=11.9 Hz, 1H), 1.73-1.60 (m, 2H), 1.60-1.43 (m, 3H), 1.29 (d, J=6.1 Hz, 3H), 1.22-1.04 (m, 4H), 1.01-0.85 (m, J=7.0 Hz, 3H), 0.83-0.73 (m, J=7.1 Hz, 1H), 0.71 (d, J=6.3 Hz, 5H), 0.59 (d, J=6.7 Hz, 3H), 0.49 (s, 3H), 0.41-0.31 (m, 2H), 0.27 (s, J=3.2, 2.8 Hz, 2H).

### Example A426. Synthesis of (3aR,6S,9S,15S,32aR)-2-acetyl-21-(5-(4-cyclopropylpiperazin-1-yl)-2-((S)-1-methoxyethyl)pyridin-3-yl)-22-ethyl-6-isopropyl-5,19,19-trimethyl-3,3a,5,6,8,9,14,15,18,19,20,22,32,32a-tetradecahydro-1H,12H-11,15-epimino-23,25-etheno-9,28-methano-26,30-(metheno)dipyrrolo[3,4-c:3′,4′-v][1,18]dioxa[6,9,12]triazacyclotriacontine-4,7,10,16(2H,13H)-tetraone

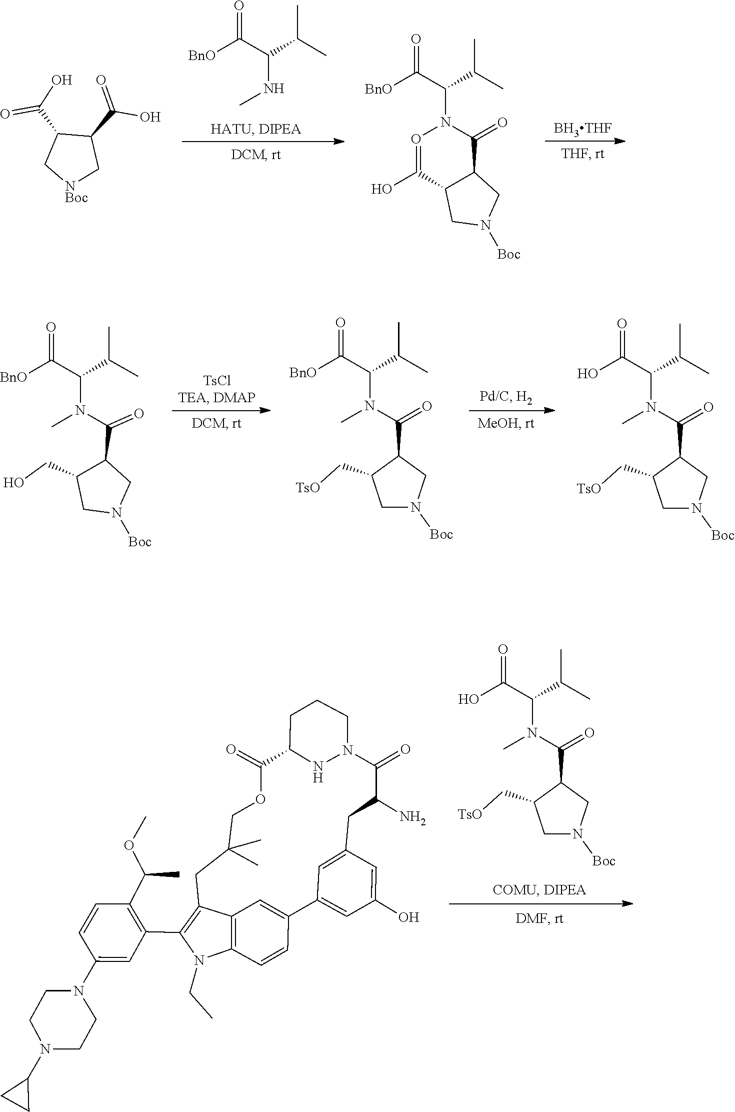

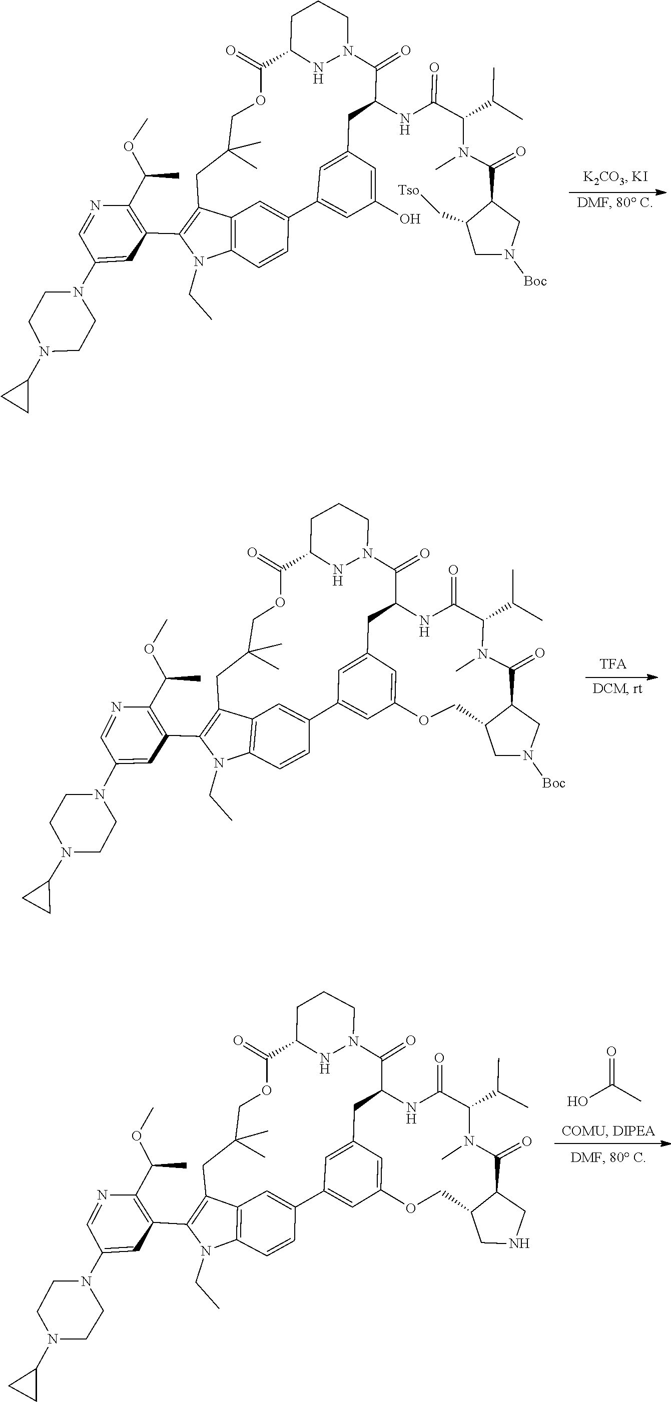

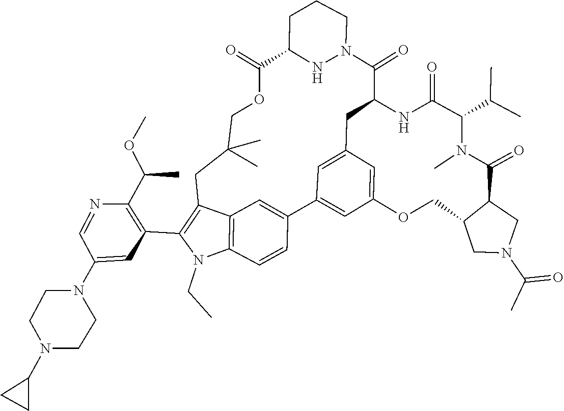

Step 1. To a solution of (3R,4R)-1-(tert-butoxycarbonyl)pyrrolidine-3,4-dicarboxylic acid (1.00 g, 3.86 mmol) in DMF (2 mL) was added HATU (1.47 g, 3.86 mmol) followed by benzyl (2S)-3-methyl-2-(methylamino)butanoate (940 mg, 4.24 mmol) at 0° C. The reaction mixture was stirred for 1 h at room temperature and was then quenched by the addition H₂O at 0° C. The aqueous mixture was extracted with DCM (3×10 mL), concentrated under reduced pressure, and purified by reversed phase chromatography to give (3R,4R)-4-{[(2S)-1-(benzyloxy)-3-methyl-1-oxobutan-2-yl](methyl)carbamoyl}-1-(tert-butoxycarbonyl)pyrrolidine-3-carboxylic acid (1.20 g, 67% yield) as a while solid. LCMS (ESI) m/z: [M−C₄H₈+H] calcd for C₂₄H₃₄N₂O₇: 407.2; found 407.2.

Step 2. To a solution of (3R,4R)-4-{[(2S)-1-(benzyloxy)-3-methyl-1-oxobutan-2-yl](methyl)carbamoyl}-1-(tert-butoxycarbonyl)pyrrolidine-3-carboxylic acid (1.00 g, 2.16 mmol) in THE (10 mL) was added BH₃·THF (370 mg, 4.32 mmol) at 0° C. The reaction mixture was stirred for 2 h at room temperature and was then quenched by the addition of H₂O at 0° C. The aqueous mixture was extracted with EtOAc (3×10 mL), concentrated under reduced pressure, and purified by normal phase chromatography to give tert-butyl (3R,4R)-3-{[(2S)-1-(benzyloxy)-3-methyl-1-oxobutan-2-yl](methyl)carbamoyl}-4-(hydroxymethyl)pyrrolidine-1-carboxylate (800 mg, 78% yield) as a yellow oil. LCMS (ESI) m/z: [M+Na] calcd for C₂₄H₃₆N₂O₆: 471.3; found 471.2.

Step 3. To a solution of tert-butyl (3R,4R)-3-{[(2S)-1-(benzyloxy)-3-methyl-1-oxobutan-2-yl](methyl)carbamoyl}-4-(hydroxymethyl)pyrrolidine-1-carboxylate (800 mg, 1.78 mmol) in DCM (8 mL) were added DMAP (21.8 mg, 0.178 mmol), TsCl (408 mg, 2.14 mmol) and TEA (541 mg, 5.35 mmol). The reaction mixture was stirred for 3 h at room temperature. The resulting mixture was washed with H₂O (3×5 mL), dried over Na₂SO₄, concentrated under reduced pressure, and purified by prep-TLC to give tert-butyl (3R,4R)-3-{[(2S)-1-(benzyloxy)-3-methyl-1-oxobutan-2-yl](methyl)carbamoyl}-4-{[(4-methylbenzenesulfonyl)oxy]methyl}pyrrolidine-1-carboxylate (900 mg, 84% yield) as a colorless oil. LCMS (ESI) m/z: [M+H] calcd for C₃₁H₄₂N₂O₈S: 603.3; found 603.3.

Step 4. To a solution of tert-butyl (3R,4R)-3-{[(2S)-1-(benzyloxy)-3-methyl-1-oxobutan-2-yl](methyl)carbamoyl}-4-{[(4-methylbenzenesulfonyl)oxy]methyl}pyrrolidine-1-carboxylate (800 mg, 1.33 mmol) in MeOH (8 mL) was added 10% Pd/C (799 mg) at room temperature under an atmosphere of argon gas. The reaction mixture was stirred for 1 h at room temperature under an atmosphere of H₂. The resulting mixture was then filtered, the filter cake washed with MeOH (3×10 mL), and the filtrate concentrated under reduced pressure to give (2S)-2-{1-[(3R,4R)-1-(tert-butoxycarbonyl)-4-{[(4-methylbenzenesulfonyl)oxy]methyl}pyrrolidin-3-yl]-N-methylformamido}-3-methylbutanoic acid (600 mg, 88%), which was used without further purification. LCMS (ESI) m/z: [M−H] calcd for C₂₄H₃₆N₂O₈S: 511.2; found 511.1.

Step 5. To a solution of (6³S,4S)-4-amino-1²-(5-(4-cyclopropylpiperazin-1-yl)-2-((S)-1-methoxyethyl)pyridin-3-yl)-1¹-ethyl-25-hydroxy-10,10-dimethyl-6¹,6²,6³,6⁴,6⁵,6⁶-hexahydro-1¹H-8-oxa-1(5,3)-indola-6(1,3)-pyridazina-2(1,3)-benzenacycloundecaphane-5,7-dione (550 mg, 0.720 mmol) in DMF (6 mL) was added DIPEA (3.72 g, 28.8 mmol) followed by (2S)-2-{1-[(3R,4R)-1-(tert-butoxycarbonyl)-4-{[(4-methylbenzenesulfonyl)oxy]methyl}pyrrolidin-3-yl]-N-methylformamido}-3-methylbutanoic acid (443 mg, 0.864 mmol) and COMU (339 mg, 0.792 mmol) at 0° C. The reaction mixture was stirred 1 h at room temperature, concentrated under reduced pressure, and purified by reversed phase chromatography to give tert-butyl (3R,4R)-3-(((2S)-1-(((6³S,4S)-1²-(5-(4-cyclopropylpiperazin-1-yl)-2-((S)-1-methoxyethyl)pyridin-3-yl)-1¹-ethyl-2⁵-hydroxy-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)amino)-3-methyl-1-oxobutan-2-yl)(methyl)carbamoyl)-4-((tosyloxy)methyl)pyrrolidine-1-carboxylate (800 mg, 84% yield) as an orange solid. LCMS (ESI) m/z: [M/2+2H] calcd for C₆₈H₉₁N₉O₁₂S: 629.8; found 630.2.

Step 6. To a solution of tert-butyl (3R,4R)-3-(((2S)-1-(((6³S,4S)-1²-(5-(4-cyclopropylpiperazin-1-yl)-2-((S)-1-methoxyethyl)pyridin-3-yl)-1¹-ethyl-2⁵-hydroxy-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)amino)-3-methyl-1-oxobutan-2-yl)(methyl)carbamoyl)-4-((tosyloxy)methyl)pyrrolidine-1-carboxylate (210 mg, 0.167 mmol) in DMF (2.1 mL) at room temperature were added KI (27.7 mg, 0.167 mmol) and K₂CO₃ (231 mg, 1.67 mmol). The reaction mixture was stirred for 2 h at 80° C. The resulting mixture was filtered, the filter cake washed with MeCN (2×2 mL) and the filtrate concentrated under reduced pressure. The residue was purified by reversed phase chromatography to give tert-butyl (3aR,6S,9S,15S,32aR)-21-(5-(4-cyclopropylpiperazin-1-yl)-2-((S)-1-methoxyethyl)pyridin-3-yl)-22-ethyl-6-isopropyl-5,19,19-trimethyl-4,7,10,16-tetraoxo-3a,4,5,6,7,8,9,10,13,14,15,16,18,19,20,22,32,32a-octadecahydro-1H,12H-11,15-epimino-23,25-etheno-9,28-methano-26,30-(metheno)dipyrrolo[3,4-c:3′,4′-v][1,18]dioxa[6,9,12]triazacyclotriacontine-2(3H)-carboxylate (154 mg, 76% yield) as a white solid. LCMS (ESI) m/z: [M+H] calcd for C₆₁H₈₃N₉O₉: 1086.6; found 1086.8.

Step 7. To a stirred solution of tert-butyl (3aR,6S,9S,15S,32aR)-21-(5-(4-cyclopropylpiperazin-1-yl)-2-((S)-1-methoxyethyl)pyridin-3-yl)-22-ethyl-6-isopropyl-5,19,19-trimethyl-4,7,10,16-tetraoxo-3a,4,5,6,7,8,9,10,13,14,15,16,18,19,20,22,32,32a-octadecahydro-1H,12H-11,15-epimino-23,25-etheno-9,28-methano-26,30-(metheno)dipyrrolo[3,4-c:3′,4′-v][1,18]dioxa[6,9,12]triazacyclotriacontine-2(3H)-carboxylate (200 mg, 0.184 mmol) in DCM (2 mL) was added TFA (0.4 mL) at 0° C. The reaction mixture was stirred for 1 h at room temperature and quenched with H₂O at 0° C. The aqueous mixture was basified to pH 8 with sat. aq. NaHCO₃, extracted with DCM (3×5 mL), dried over Na₂SO₄, filtered, and concentrated under reduced pressure to give (3aR,6S,9S,15S,32aR)-21-(5-(4-cyclopropylpiperazin-1-yl)-2-((S)-1-methoxyethyl)pyridin-3-yl)-22-ethyl-6-isopropyl-5,19,19-trimethyl-3,3a,5,6,8,9,14,15,18,19,20,22,32,32a-tetradecahydro-1H,12H-11,15-epimino-23,25-etheno-9,28-methano-26,30-(metheno)dipyrrolo[3,4-c:3′,4′-v][1,18]dioxa[6,9,12]triazacyclotriacontine-4,7,10,16(2H,13H)-tetraone (215 mg, crude), which was used without further purification. LCMS (ESI) m/z: [M+H] calcd for C₅₆H₇₅N₉O₇: 986.6; found 986.6.

Step 8. To a solution of (3aR,6S,9S,15S,32aR)-21-(5-(4-cyclopropylpiperazin-1-yl)-2-((S)-1-methoxyethyl)pyridin-3-yl)-22-ethyl-6-isopropyl-5,19,19-trimethyl-3,3a,5,6,8,9,14,15,18,19,20,22,32,32a-tetradecahydro-1H,12H-11,15-epimino-23,25-etheno-9,28-methano-26,30-(metheno)dipyrrolo[3,4-c:3′,4′-v][1,18]dioxa[6,9,12]triazacyclotriacontine-4,7,10,16(2H,13H)-tetraone (97 mg, crude) in DMF was added DIPEA (127 mg, 0.980 mmol) followed by acetic acid (11.8 mg 0.296 mmol) and COMU (54.8 mg, 0.127 mmol) at 0° C. The reaction mixture was stirred for 1 h at room temperature and was quenched by the addition of H₂O (5 mL) at 0° C. The resulting mixture was extracted with DCM (3×10 mL), dried over Na₂SO₄, filtered, and concentrated under reduced pressure. The crude product was purified by reversed phase chromatography to give (3aR,6S,9S,15S,32aR)-2-acetyl-21-(5-(4-cyclopropylpiperazin-1-yl)-2-((S)-1-methoxyethyl)pyridin-3-yl)-22-ethyl-6-isopropyl-5,19,19-trimethyl-3,3a,5,6,8,9,14,15,18,19,20,22,32,32a-tetradecahydro-1H,12H-11,15-epimino-23,25-etheno-9,28-methano-26,30-(metheno)dipyrrolo[3,4-c:3′,4′-v][1,18]dioxa[6,9,12]triazacyclotriacontine-4,7,10,16(2H,13H)-tetraone (44 mg, 0.0428 mmol 52% yield over 2 steps) as a white solid. LCMS (ESI) m/z: [M+H] calcd for C₅₈H₇₇N₉O₈: 1028.6; found 1028.5. ¹H NMR (400 MHz, DMSO-d₆) δ 8.38 (d, J=2.8 Hz, 1H), 8.29 (t, J=7.1 Hz, 1H), 7.89 (s, 1H), 7.64-7.55 (m, 1H), 7.52-7.47 (m, 1H), 7.27 (s, 1H), 7.18-7.05 (m, 2H), 6.72-6.63 (m, 1H), 5.20-5.12 (m, 2H), 4.78-4.69 (m, 1H), 4.43-4.34 (m, 1H), 4.26-3.94 (m, 6H), 3.90-3.80 (m, 1H), 3.78-3.71 (m, 1H), 3.63-3.55 (m, 2H), 3.45-3.40 (m, 1H), 3.26-3.10 (m, 6H), 3.09-3.02 (m, 3H), 2.91-2.67 (m, 5H), 2.66-2.56 (m, 7H), 2.54-2.46 (m, 1H), 2.04-1.95 (m, 2H), 1.90 (d, J=18.4 Hz, 3H), 1.80-1.74 (m, 1H), 1.70-1.61 (m, 1H), 1.62-1.56 (m, 1H), 1.54-1.42 (m, 1H), 1.27 (d, J=6.1 Hz, 3H), 0.87 (q, J=10.4, 8.7 Hz, 3H), 0.81-0.71 (m, 6H), 0.67 (d, J=6.6 Hz, 3H), 0.50-0.31 (m, 5H), 0.30-0.23 (m, 2H).

### Example A428. Synthesis of N-((9S,15S,18S,21S)-3-(5-(4-cyclopropylpiperazin-1-yl)-2-((S)-1-methoxyethyl)pyridin-3-yl)-2-ethyl-18-isopropyl-5,5,19-trimethyl-8,14,17,20-tetraoxo-2,4,5,6,9,10,11,12,14,15,16,17,18,19,20,21,22,23-octadecahydro-8H-9,13-epimino-1,30-etheno-15,27-methano-25,29-(metheno)pyrrolo[3,4-v][1,18]dioxa[6,9,12]triazacyclotriacontin-21-yl)acetamide

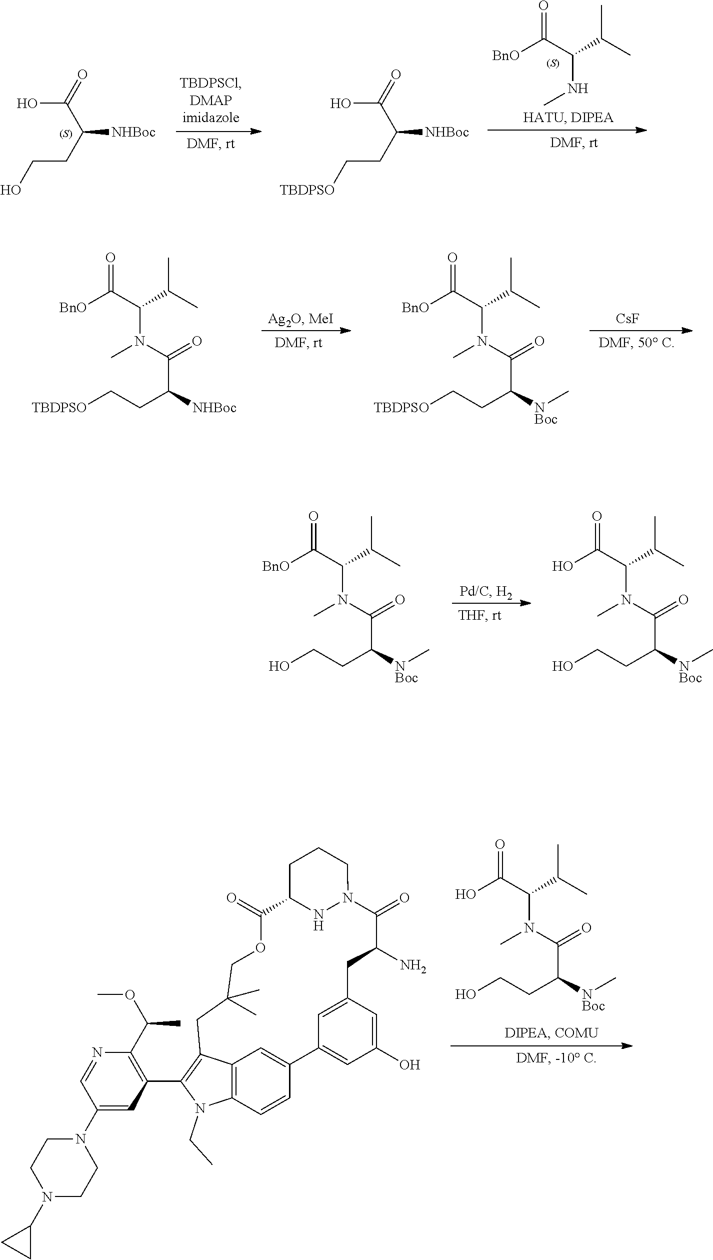

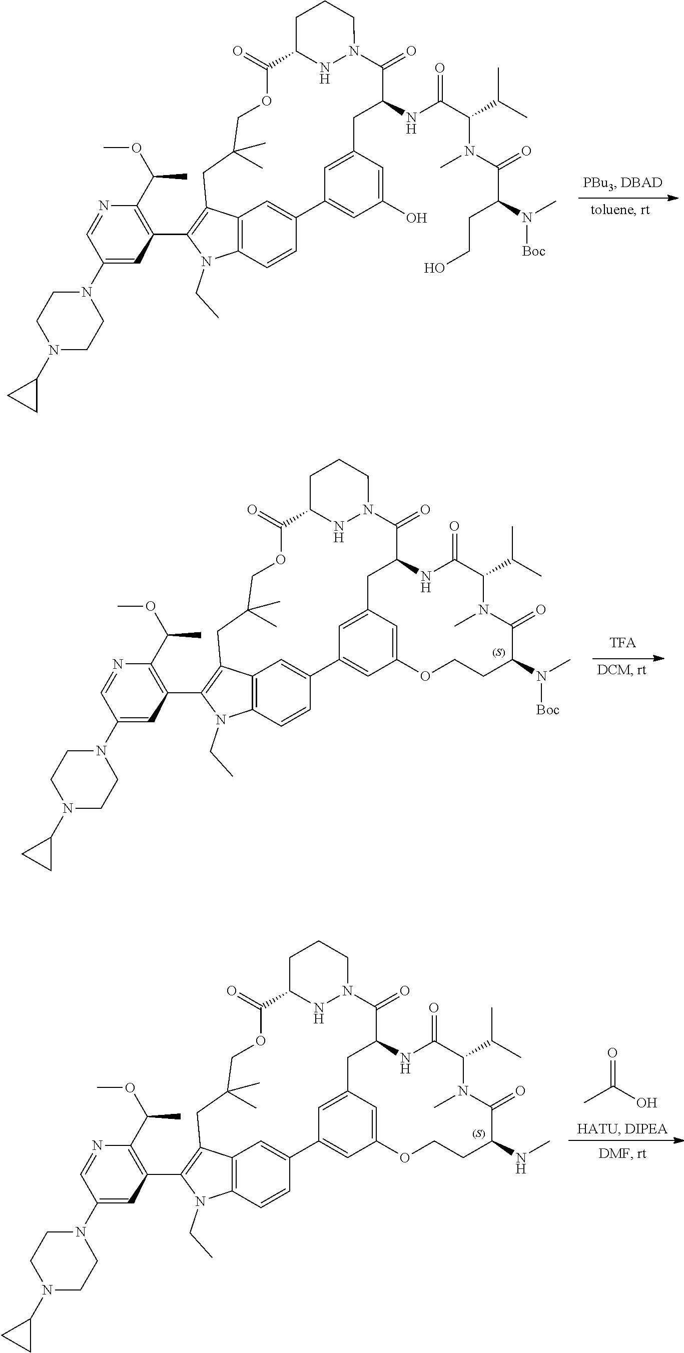

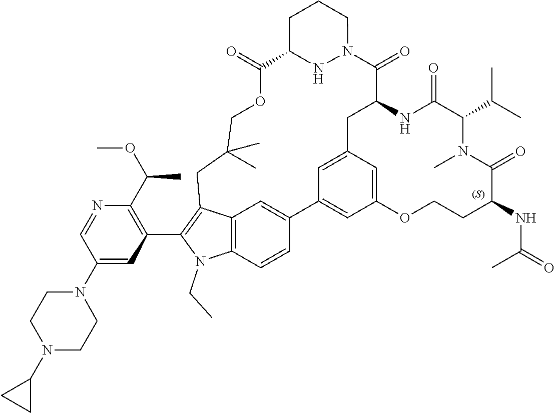

Step 1. To a solution of (2S)-2-[(tert-butoxycarbonyl)amino]-4-hydroxybutanoic acid (15.0 g, 68.4 mmol) in DCM (150 ml) were added imidazole (11.6 g, 170 mmol), TBDPSCI (28.2 g, 103 mmol), and DMAP (1.67 g, 13.7 mmol) at 0° C. The resulting mixture was stirred for 16 h at room temperature and was then extracted with DCM (3×1 L), treated with brine (3×2 L), dried over Na₂SO₄, filtered, and concentrated under reduced pressure to give (2S)-2-[(tert-butoxycarbonyl)amino]-4-[(tert-butyldiphenylsilyl)oxy]butanoic acid (30.0 g, crude), which was used without further purification. LCMS (ESI) m/z: [M+H] calcd for C₂₅H₃₅NO₅Si: 458.2; found 458.1.

Step 2. To a solution of (2S)-2-[(tert-butoxycarbonyl)amino]-4-[(tert-butyldiphenylsilyl)oxy]butanoic acid (30.0 g, crude) and benzyl (2S)-3-methyl-2-(methylamino)butanoate (14.5 g, 65.6 mmol) in DMF (100 mL) were added DIPEA (84.7 g, 656 mmol) and HATU (32.4 g, 85.2 mmol) in DMF (200 mL) at 0° C. The resulting mixture was stirred for 2 h at room temperature and was then quenched by the addition of H₂O. The aqueous mixture was extracted with EtOAc (3×1 L) and the combined organic extracts were treated with brine (3×2 L), dried over Na₂SO₄, filtered, and concentrated under reduced pressure. The residue was purified by normal phase chromatography to afford benzyl (2S)-2-[(2S)-2-[(tert-butoxycarbonyl)amino]-4-[(tert-butyldiphenylsilyl)oxy]-N-methylbutanamido]-3-methylbutanoate (30.6 g, 68% yield over 2 steps) as a yellow oil. LCMS (ESI) m/z: [M+H] calcd for C₃₈H₅₂N₂O₆Si: 661.4; found 661.3.

Step 3. To a solution of benzyl (2S)-2-[(2S)-2-[(tert-butoxycarbonyl)amino]-4-[(tert-butyldiphenylsilyl)oxy]-N-methylbutanamido]-3-methylbutanoate (30.6 g, 46.3 mmol) and Ag₂O (53.7 g, 231 mmol) in DMF (120 mL) was added Mel (197 g, 1390 mmol) at 0° C. The resulting mixture was stirred for 16 h at 30° C. and was then filtered and the filter cake washed with EtOAc (3×1 L). The filtrate was treated with brine (3×2 L), dried over Na₂SO₄, filtered, and concentrated under reduced pressure. The residue was purified by reversed phase chromatography to afford benzyl (2S)-2-[(2S)-2-[(tert-butoxycarbonyl)(methyl)amino]-4-[(tert-butyldiphenylsilyl)oxy]-N-methylbutanamido]-3-methylbutanoate (29.0 g, 77% yield) as a yellow oil. LCMS (ESI) m/z: [M+H] calcd for C₃₉H₅₄N₂O₆Si: 675.4; found 675.3.

Step 4. To a solution of benzyl (2S)-2-[(2S)-2-[(tert-butoxycarbonyl)(methyl)amino]-4-[(tert-butyldiphenylsilyl)oxy]-N-methylbutanamido]-3-methylbutanoate (28.8 g, 42.7 mmol) in DMF (288 mL) was added CsF (32.4 g, 213 mmol) at room temperature. The resulting mixture was stirred for 16 h at 50° C. and was then quenched by the addition of H₂O. The aqueous mixture was extracted with EtOAc (3×1 L) and the combined organic extracts were washed with brine (3×2 L), dried over Na₂SO₄, filtered, and concentrated under reduced pressure. The residue was purified by reversed-phase chromatography to afford benzyl (2S)-2-[(2S)-2-[(tert-butoxycarbonyl)(methyl)amino]-4-hydroxy-N-methylbutanamido]-3-methylbutanoate (4.50 g, 24% yield) as a white solid. LCMS (ESI) m/z: [M+H] calcd for C₂₃H₃₆N₂O₆: 437.3; found 437.1.

Step 5. To a solution of benzyl (2S)-2-[(2S)-2-[(tert-butoxycarbonyl)(methyl)amino]-4-hydroxy-N-methylbutanamido]-3-methylbutanoate (3.98 g, 9.12 mmol) in THE (40 mL) was added 10% Pd/C (2.00 g) under an atmosphere of N₂. The resulting mixture was stirred for 2 h at room temperature under an atmosphere of H₂ and was then filtered, the filter cake washed with MeOH (3×100 mL), and the filtrate concentrated under reduced pressure to afford (2S)-2-[(2S)-2-[(tert-butoxycarbonyl)(methyl)amino]-4-hydroxy-N-methylbutanamido]-3-methylbutanoic acid (3.0 g, crude), which was used without further purification. LCMS (ESI) m/z: [M+H] calcd for C₁₆H₃₀N₂O₆: 347.2; found 347.1.

Step 6. To a solution of (6³S,4S)-4-amino-1²-(5-(4-cyclopropylpiperazin-1-yl)-2-((S)-1-methoxyethyl)pyridin-3-yl)-1¹-ethyl-25-hydroxy-10,10-dimethyl-6¹,6²,6³,6⁴,6⁵,6⁶-hexahydro-1¹H-8-oxa-1(5,3)-indola-6(1,3)-pyridazina-2(1,3)-benzenacycloundecaphane-5,7-dione (3.46 g, 4.53 mmol) in DMF (15 mL) were added DIPEA (23.4 g, 181 mmol) and (2S)-2-[(2S)-2-[(tert-butoxycarbonyl)(methyl)amino]-4-hydroxy-N-methylbutanamido]-3-methylbutanoic acid (2.04 g, crude) followed by a solution of COMU (2.52 g, 5.89 mmol) in DMF (20 mL) at −15° C. The resulting mixture was stirred for 1 h at −10° C. and was then quenched by the addition of H₂O at 0° C. The aqueous mixture was extracted with EtOAc (3×300 mL), treated with brine (3×300 mL), dried over Na₂SO₄, filtered, and concentrated under reduced pressure. The residue was purified by reversed phase chromatography to afford tert-butyl ((2S)-1-(((2S)-1-(((6³S,4S)-12-(5-(4-cyclopropylpiperazin-1-yl)-2-((S)-1-methoxyethyl)pyridin-3-yl)-1¹-ethyl-2⁵-hydroxy-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)- benzenacycloundecaphane-4-yl)amino)-3-methyl-1-oxobutan-2-yl)(methyl)amino)-4-hydroxy-1-oxobutan-2-yl)(methyl)carbamate (3.10 g, 56% yield) as a yellow solid. LCMS (ESI) m/z: [M+H] calcd for C₆₀H₈₅N₉O₁₀: 1092.6; found 1092.9.

Step 7. To a solution of tert-butyl ((2S)-1-(((2S)-1-(((6³S,4S)-12-(5-(4-cyclopropylpiperazin-1-yl)-2-((S)-1-methoxyethyl)pyridin-3-yl)-1¹-ethyl-2⁵-hydroxy-10,10-dimethyl-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-4-yl)amino)-3-methyl-1-oxobutan-2-yl)(methyl)amino)-4-hydroxy-1-oxobutan-2-yl)(methyl)carbamate (3.20 g, 2.93 mmol) in toluene (10 mL) was added tributylphosphine (2.96 g, 14.6 mmol) followed by a solution of DBAD (3.37 g, 14.6 mmol) in toluene (22 mL) at 0° C. The resulting mixture was stirred for 16 h at room temperature and was then quenched by the addition of salted ice (600 mL) at room temperature. The aqueous phase was extracted with EtOAc (3×400 mL) and the combined organic extracts were treated with brine (3×300 mL), dried over Na₂SO₄, filtered, and concentrated under reduced pressure. The residue was purified by reversed phase chromatography to afford tert-butyl ((9S,15S,18S,21S)-3-(5-(4-cyclopropylpiperazin-1-yl)-2-((S)-1-methoxyethyl)pyridin-3-yl)-2-ethyl-18-isopropyl-5,5,19-trimethyl-8,14,17,20-tetraoxo-2,4,5,6,9,10,11,12,14,15,16,17,18,19,20,21,22,23-octadecahydro-8H-9,13-epimino-1,30-etheno-15,27-methano-25,29-(metheno)pyrrolo[3,4-v][1,18]dioxa[6,9,12]triazacyclotriacontin-21-yl)(methyl)carbamate (2.10 g, 58% yield) as a white solid. LCMS (ESI) m/z: [M+H] calcd for C₆₀H₈₃N₉O₉: 1074.6; found 1074.7.

Step 8. To a solution of tert-butyl ((9S,15S,18S,21S)-3-(5-(4-cyclopropylpiperazin-1-yl)-2-((S)-1-methoxyethyl)pyridin-3-yl)-2-ethyl-18-isopropyl-5,5,19-trimethyl-8,14,17,20-tetraoxo-2,4,5,6,9,10,11,12,14,15,16,17,18,19,20,21,22,23-octadecahydro-8H-9,13-epimino-1,30-etheno-15,27-methano-25,29-(metheno)pyrrolo[3,4-v][1,18]dioxa[6,9,12]triazacyclotriacontin-21-yl)(methyl)carbamate (165 mg, 0.154 mmol) in DCM (1.2 mL) was added TFA (0.4 mL) at 0° C. The resulting mixture was stirred for 1 h at room temperature and was then diluted with DCM (10 mL) and basified to pH 8 by the addition of sat. aq. NaHCO₃. The aqueous phase was further extracted with DCM (3×5 mL), dried over Na₂SO₄, filtered, and concentrated under reduced pressure to afford (9S,15S,18S,21S)-3-(5-(4-cyclopropylpiperazin-1-yl)-2-((S)-1-methoxyethyl)pyridin-3-yl)-2-ethyl-18-isopropyl-5,5,19-tri methyl-21-(methylamino)-2,4,5,6,9,10,11,12,15,16,18,19,22,23-tetradecahydro-8H-9,13-epimino-1,30-etheno-15,27-methano-25,29-(metheno)pyrrolo[3,4-v][1,18]dioxa[6,9,12]triazacyclotriacontine-8,14,17,20(21H)-tetraone (150 mg, crude), which was used without further purification. LCMS (ESI) m/z: [M+H] calcd for C₅₅H₇₅N₉O₇: 974.6; found 974.5.

Step 9. To a solution of (9S,15S,18S,21S)-3-(5-(4-cyclopropylpiperazin-1-yl)-2-((S)-1-methoxyethyl)pyridin-3-yl)-2-ethyl-18-isopropyl-5,5,19-trimethyl-21-(methylamino)-2,4,5,6,9,10,11,12,15,16,18,19,22,23-tetradecahydro-8H-9,13-epimino-1,30-etheno-15,27-methano-25,29-(metheno)pyrrolo[3,4-v][1,18]dioxa[6,9,12]triazacyclotriacontine-8,14,17,20(21H)-tetraone (70.0 mg, crude) in DMF (1 mL) were added DIPEA (92.9 mg, 0.720 mmol), acetic acid (5.61 mg, 0.094 mmol) and HATU (35.5 mg, 0.094 mmol) at 0° C. The resulting mixture was stirred for 2 h at room temperature and was then quenched by the addition of H₂O at 0° C. The aqueous mixture was extracted with EtOAc (3×5 mL) and the combined organic extracts were treated with brine (3×5 mL), dried over Na₂SO₄, filtered, and concentrated under reduced pressure. The crude product was purified by reversed phase chromatography to afford N-((9S,15S,18S,21 S)-3-(5-(4-cyclopropylpiperazin-1-yl)-2-((S)-1-methoxyethyl)pyridin-3-yl)-2-ethyl-18-isopropyl-5,5,19-trimethyl-8,14,17,20-tetraoxo-2,4,5,6,9,10,11,12,14,15,16,17,18,19,20,21,22,23-octadecahydro-8H-9,13-epimino-1,30-etheno-15,27-methano-25,29-(metheno)pyrrolo[3,4-v][1,18]dioxa[6,9,12]triazacyclotriacontin-21-yl)acetamide (19.4 mg, 27% yield over 2 steps) as a white solid. LCMS (ESI) m/z: [M+H] calcd for C₅₆H₇₅N₉O₈: 1016.6; found 1016.6. ¹H NMR (400 MHz, DMSO-d₆) δ 8.70-8.30 (m, 2H), 8.15-7.85 (m, 1H), 7.85-7.65 (m, 1H), 7.63-7.50 (m, 1H), 7.50-7.30 (m, 1H), 7.28-6.91 (m, 1H), 6.88-6.54 (m, 1H), 5.60-5.10 (m, 3H), 4.96-4.69 (m, 1H), 4.65-4.40 (m, 1H), 4.36-3.90 (m, 6H), 3.88-3.55 (m, 3H), 3.31-3.14 (m, 4H), 3.10-3.00 (m, 3H), 2.95-2.72 (m, 7H), 2.70-2.54 (m, 6H), 2.36-2.18 (m, 1H), 2.16-2.05 (m, 1H), 2.03-1.90 (m, 4H), 1.89-1.69 (m, 3H), 1.68-1.45 (m, 3H), 1.41-1.23 (m, 3H), 1.10-0.89 (m, 3H), 0.89-0.64 (m, 9H), 0.59-0.48 (m, 3H), 0.47-0.37 (m, 2H), 0.34-0.20 (m, 2H).

### Example A432. Synthesis of (9S,15S,18S,20aR,23S)-3-(5-(4-cyclopropylpiperazin-1-yl)-2-((S)-1-methoxyethyl)pyridin-3-yl)-2-ethyl-18-isopropyl-N,N,5,5,19-pentamethyl-8,14,17,20-tetraoxo-2,4,5,6,9,10,11,12,15,16,17,18,19,20,20a,21,22,23,25,26-icosahydro-8H,14H-9,13-epimino-1,32-etheno-15,29-methano-27,31-(metheno)dipyrrolo[2,1-o:3′,4′-z][1]oxa[7,10,13,16]tetraazacyclotriacontine-23-carboxamide

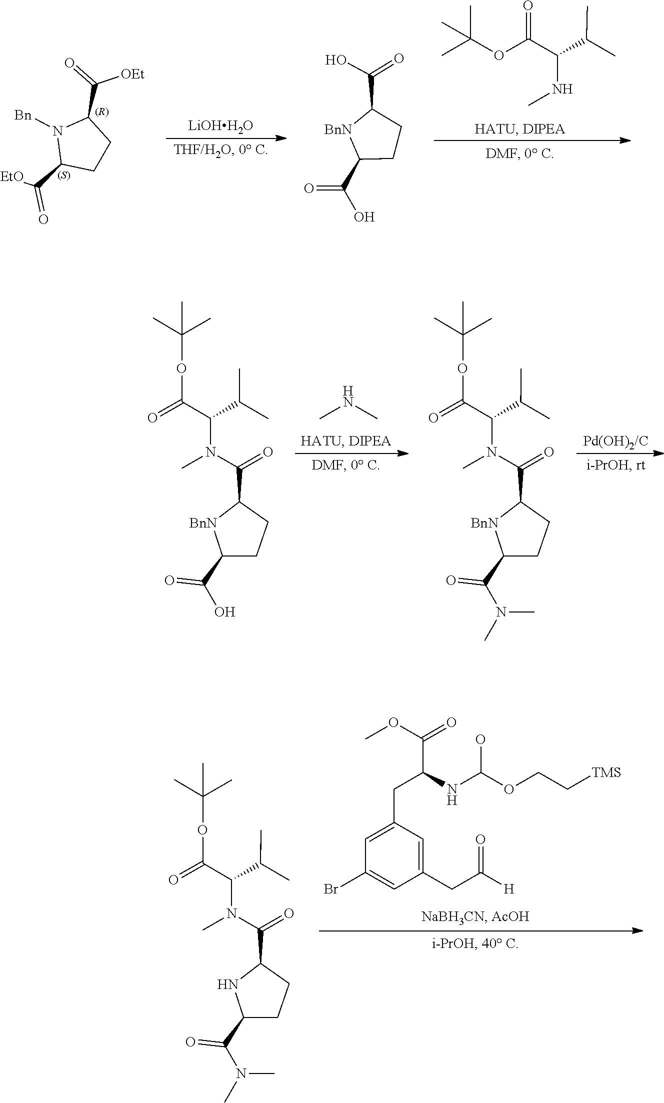

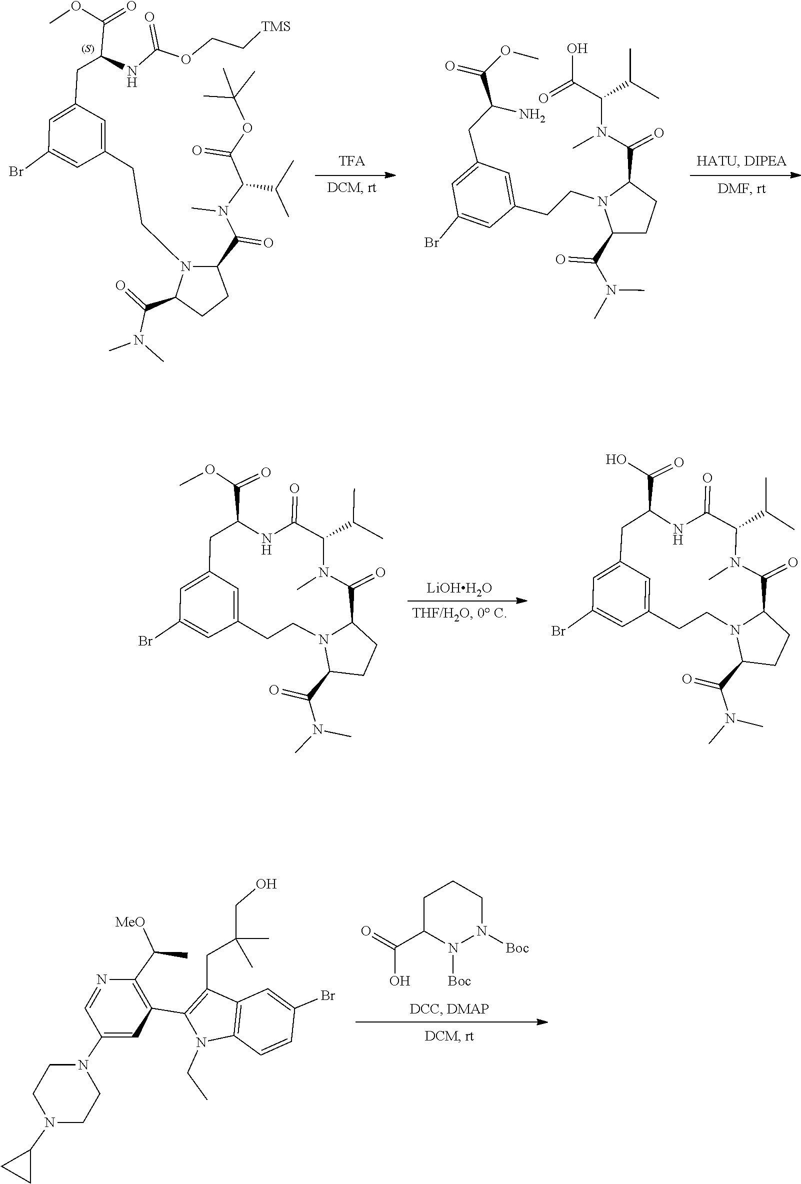

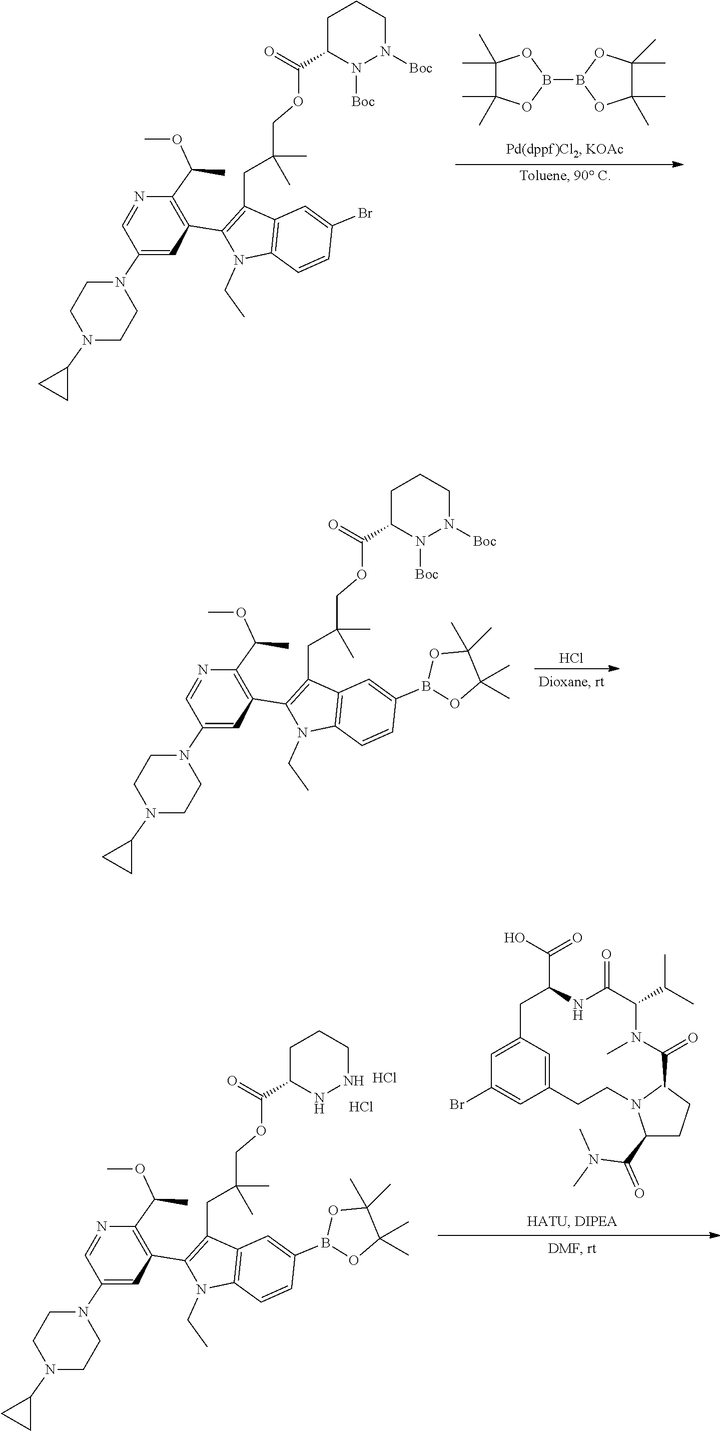

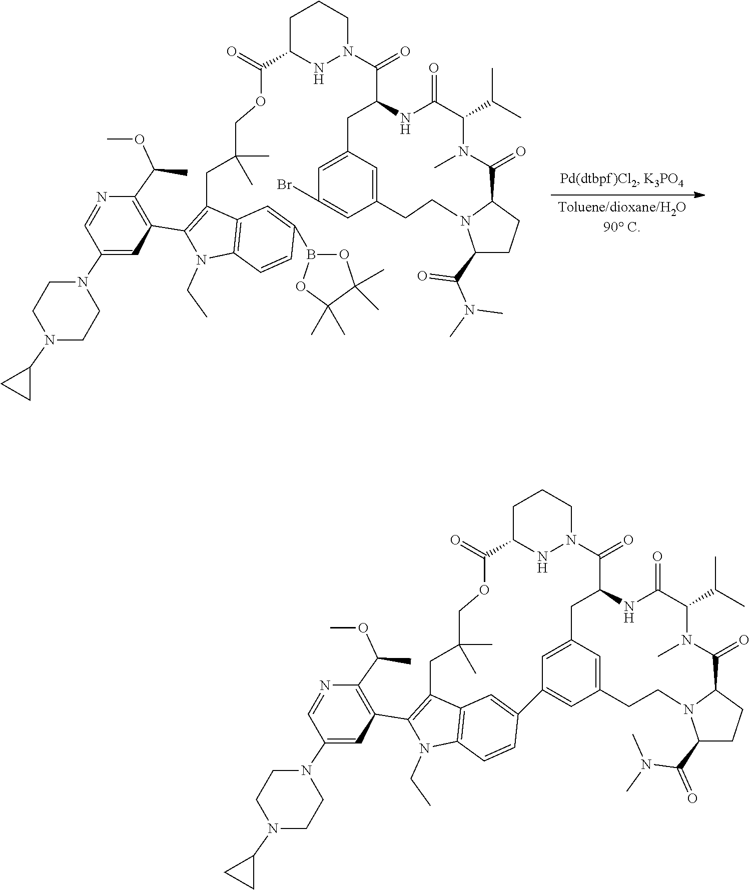

Step 1. To a solution of diethyl (2R,5S)-1-benzylpyrrolidine-2,5-dicarboxylate (2.00 g, 6.55 mmol) in THE (10 mL) and H₂O (10 mL) was added LiOH·H₂O (1.10 g, 26.2 mmol) at 0° C. The resulting mixture was stirred for 2 h at 0° C. and was then acidified to pH 6 by the addition of 1 N aq. HCl. The aqueous mixture was extracted with EtOAc (3×50 mL) and the combined organic extracts were washed with brine (50 mL), dried over Na₂SO₄, filtered, and concentrated under reduced pressure to afford (2R,5S)-1-benzylpyrrolidine-2,5-dicarboxylic acid (1.50 g, crude), which was used without further purification. LCMS (ESI) m/z: [M+H] calcd for C₁₃H₁₅NO₄: 250.1; found 250.3.

Step 2. To a solution of (2R,5S)-1-benzylpyrrolidine-2,5-dicarboxylic acid (1.50 g, crude) and tert-butyl (2S)-3-methyl-2-(methylamino)butanoate (1.01 g, 5.42 mmol) in DMF (15 mL) were added HATU (2.75 g, 7.22 mmol) and DIPEA (2.33 g, 18.1 mmol) 0° C. The resulting mixture was stirred for 2 h at 0° C. and was then quenched by the addition of H₂O (20 mL) at room temperature. The resulting mixture was extracted with EtOAc (3×50 mL) and the combined organic extracts were washed with brine (3×50 mL), dried over Na₂SO₄, filtered, and concentrated under reduced pressure. The residue was purified by reversed phase chromatography to afford (2S,5R)-1-benzyl-5-(((S)-1-(tert-butoxy)-3-methyl-1-oxobutan-2-yl)(methyl)carbamoyl)pyrrolidine-2-carboxylic acid (960 mg, 35% yield over 2 steps) as a colorless oil. LCMS (ESI) m/z: [M+H] calcd for C₂₃H₃₄N₂O₅: 419.3; found 419.4.

Step 3. To a solution of (2S,5R)-1-benzyl-5-(((S)-1-(tert-butoxy)-3-methyl-1-oxobutan-2-yl)(methyl)carbamoyl)pyrrolidine-2-carboxylic acid (960 mg, 2.29 mmol) and dimethylamine HCl salt (374 mg, 4.59 mmol) in DMF (10 mL) were added HATU (1.74 g, 4.59 mmol) and DIPEA (1.48 g, 11.5 mmol) at 0° C. The resulting mixture was stirred for 2 h at 0° C. and was then quenched by the addition of H₂O (20 mL) at room temperature. The aqueous mixture was extracted with EtOAc (3×50 mL), and the combined organic extracts were washed with brine (3×50 mL), dried over Na₂SO₄, filtered, and concentrated under reduced pressure. The residue was purified by reversed phase chromatography to afford tert-butyl N-((2R,5S)-1-benzyl-5-(dimethylcarbamoyl)pyrrolidine-2-carbonyl)-N-methyl-L-valinate (840 mg, 82% yield) as a yellow oil. LCMS (ESI) m/z: [M+H] calcd for C₂₅H₃₉N₃O₄: 446.3; found 446.4.

Step 4. To a solution of tert-butyl N-((2R,5S)-1-benzyl-5-(dimethylcarbamoyl)pyrrolidine-2-carbonyl)-N-methyl-L-valinate (830 mg, 1.86 mmol) in i-PrOH (8 mL) was added 10% Pd(OH)₂/C (829 mg) under an atmosphere of N₂. The resulting mixture was stirred for 1 h at room temperature under an atmosphere of H₂ and was then filtered through a Celite pad. The filter cake washed with MeOH (3×30 mL), and the filtrate concentrated under reduced pressure to afford tert-butyl N-((2R,5S)-5-(dimethylcarbamoyl)pyrrolidine-2-carbonyl)-N-methyl-L-valinate (700 mg, crude), which was used without further purification. LCMS (ESI) m/z: [M+H] calcd for C₁₈H₃₃N₃O₄: 356.3; found 356.2.

Step 5. To a solution of tert-butyl N-((2R,5S)-5-(dimethylcarbamoyl)pyrrolidine-2-carbonyl)-N-methyl-L-valinate (1.03 g, crude) and methyl (S)-3-(3-bromo-5-(2-oxoethyl)phenyl)-2-(((2-(trimethylsilyl)ethoxy)carbonyl)amino)propanoate (1.67 g, crude) in i-PrOH (10 mL) were added AcOH (522 mg, 8.69 mmol) and NaBH₃CN (546 mg, 8.69 mmol) at 0° C. The resulting mixture was stirred overnight at 40° C. and was then quenched by the addition of H₂O (20 mL) at room temperature. The aqueous mixture was extracted with EtOAc (3×50 mL) and the combined organic extracts were washed with brine (3×50 mL), dried over Na₂SO₄, filtered, and concentrated under reduced pressure. The residue was purified by reversed phase chromatography to afford tert-butyl N-((2R,5S)-1-(3-bromo-5-((S)-3-methoxy-3-oxo-2-(((2-(trimethylsilyl)ethoxy)carbonyl)amino)propyl)phenethyl)-5-(dimethylcarbamoyl)pyrrolidine-2-carbonyl)-N-methyl-L-valinate (1.40 g, 65% yield over 2 steps) as a yellow oil. LCMS (ESI) m/z: [M+H] calcd for C₃₆H₅₉BrN₄₀₈Si: 783.3; found 783.4.

Step 6. To a solution of tert-butyl N-((2R,5S)-1-(3-bromo-5-((S)-3-methoxy-3-oxo-2-(((2-(trimethylsilyl)ethoxy)carbonyl)amino)propyl)phenethyl)-5-(dimethylcarbamoyl)pyrrolidine-2-carbonyl)-N-methyl-L-valinate (1.40 g, 1.79 mmol) in DCM (14 mL) was added TFA (14 mL) at 0° C. The resulting mixture was stirred for 1 h at room temperature and was then diluted with toluene (30 mL) and concentrated under reduced pressure to afford N-((2R,5S)-1-(3-((S)-2-amino-3-methoxy-3-oxopropyl)-5-bromophenethyl)-5-(dimethylcarbamoyl)pyrrolidine-2-carbonyl)-N-methyl-L-valine (2.00 g, crude), which was used without further purification. LCMS (ESI) m/z: [M+H] calcd for C₂₆H₃₉BrN₄O₆: 583.2; found 583.1.

Step 7. To a solution of N-((2R,5S)-1-(3-((S)-2-amino-3-methoxy-3-oxopropyl)-5-bromophenethyl)-5-(dimethylcarbamoyl)pyrrolidine-2-carbonyl)-N-methyl-L-valine (2.00 g, crude) and DIPEA (4.43 g, 34.3 mmol) in DMF (200 mL) was added HATU (2.61 g, 6.85 mmol) at 0° C. The resulting mixture was stirred for 1 h at room temperature and was then quenched by the addition of H₂O (300 mL) at 0° C. The aqueous mixture was extracted with EtOAc (3×200 mL) and the combined organic extracts were washed with brine (3×200 mL), dried over Na₂SO₄, filtered, and concentrated under reduced pressure. The residue was purified by reversed phase chromatography to afford methyl (1²R,1⁵S,6S,9S)-4⁵-bromo-1⁵-(dimethylcarbamoyl)-9-isopropyl-1 O-methyl-8,11-dioxo-7,10-diaza-1 (1,2)-pyrrolidina-4(1,3)-benzenacycloundecaphane-6-carboxylate (450 mg, 44% yield over 2 steps) as a yellow oil. LCMS (ESI) m/z: [M+H] calcd for C₂₆H₃₇BrN₄O₅: 565.1; found 565.2.

Step 8. To a stirred solution of methyl (1²R,1⁵S,6S,9S)-4⁵-bromo-1⁵-(dimethylcarbamoyl)-9-isopropyl-10-methyl-8,11-dioxo-7,10-diaza-1(1,2)-pyrrolidina-4(1,3)-benzenacycloundecaphane-6-carboxylate (450 mg, 0.796 mmol) in H₂O (3 mL) and THE (3 mL) was added LiOH·H₂O (66.8 mg, 1.59 mmol) at 0° C. The resulting mixture was stirred for 1 h at room temperature and was then acidified to pH 6 by the addition of 1 N aq. HCl. The aqueous mixture was extracted with DCM (3×20 mL), and the combined organic extracts were dried over anhydrous Na₂SO₄, filtered, and concentrated under reduced pressure. The residue was purified by reversed phase chromatography to afford (1²R,1⁵S,6S,9S)-4⁵-bromo-1⁵-(dimethylcarbamoyl)-9-isopropyl-10-methyl-8,11-dioxo-7,10-diaza-1(1,2)-pyrrolidina-4(1,3)-benzenacycloundecaphane-6-carboxylic acid (251 mg, 53% yield) as a yellow oil. LCMS (ESI) m/z: [M+H] calcd for C₂₅H₃₅BrN₄O₅: 551.2; found 551.2.

Step 9. To a stirred solution of (S)-3-(5-bromo-2-(5-(4-cyclopropylpiperazin-1-yl)-2-(1-methoxyethyl)pyridin-3-yl)-1-ethyl-1H-indol-3-yl)-2,2-dimethylpropan-1-ol (10.0 g, 17.6 mmol) and (S)-1,2-bis(tert-butoxycarbonyl)hexahydropyridazine-3-carboxylic acid (6.96 g, 21.1 mmol) in DCM (100 mL) were added DMAP (210 mg, 1.76 mmol) and DCC (5.43 g, 26.3 mmol) at 0° C. The resulting mixture was stirred overnight at room temperature and was then quenched by the addition of H₂O (100 mL). The aqueous phase was further extracted with DCM (3×100 mL) and the combined organic extracts were dried over anhydrous Na₂SO₄, filtered, and concentrated under reduced pressure. The residue was purified by normal phase chromatography to afford 3-(3-(5-bromo-2-(5-(4-cyclopropylpiperazin-1-yl)-2-((S)-1-methoxyethyl)pyridin-3-yl)-1-ethyl-1H-indol-3-yl)-2,2-dimethylpropyl)1,2-di-tert-butyl-(S)-tetrahydropyridazine-1,2,3-tricarboxylate (15.0 g, 87% yield) as a yellow solid. LCMS (ESI) m/z: [M+H] calcd for C₄₅H₆₅BrN₆O₇: 881.4; found 881.4.

Step 10. To a solution of 3-(3-(5-bromo-2-(5-(4-cyclopropylpiperazin-1-yl)-2-((S)-1-methoxyethyl)pyridin-3-yl)-1-ethyl-1H-indol-3-yl)-2,2-dimethylpropyl)-1,2-di-tert-butyl-(S)-tetrahydropyridazine-1,2,3-tricarboxylate (3.30 g, 3.74 mmol) and 4,4,5-trimethyl-2-(4,4,5-trimethyl-1,3,2-dioxaborolan-2-yl)-1,3,2-dioxaborolane (1.27 g, 5.61 mmol) in toluene (30 mL) were added KOAc (734 mg, 7.48 mmol) and Pd(dppfCl₂ (305 mg, 0.374 mmol) at room temperature. The resulting mixture was stirred overnight at 80° C. under an atmosphere of N₂ and was then quenched by the addition of H₂O (20 mL) at room temperature. The aqueous phase was extracted with EtOAc (3×100 mL) and the combined organic extracts were washed with brine (3×100 mL), dried over anhydrous Na₂SO₄, filtered, and concentrated under reduced pressure. The residue was purified by normal phase chromatography to afford 1,2-di-tert-butyl 3-(3-(2-(5-(4-cyclopropylpiperazin-1-yl)-2-((S)-1-methoxyethyl)pyridin-3-yl)-1-ethyl-5-(4,4,5,5-tetramethyl-1,3,2-dioxaborolan-2-yl)-1H-indol-3-yl)-2,2-dimethylpropyl)-(S)-tetrahydropyridazine-1,2,3-tricarboxylate (2.60 g, 75% yield) as a yellow oil. LCMS (ESI) m/z: [M+H] calcd for C₅₁H₇₇BN₆O₉: 929.6; found 929.3.

Step 11. To a solution of 1,2-di-tert-butyl 3-(3-(2-(5-(4-cyclopropylpiperazin-1-yl)-2-((S)-1-methoxyethyl)pyridin-3-yl)-1-ethyl-5-(4,4,5,5-tetramethyl-1,3,2-dioxaborolan-2-yl)-1H-indol-3-yl)-2,2-dimethylpropyl) (S)-tetrahydropyridazine-1,2,3-tricarboxylate (1.00 g, 1.08 mmol) in dioxane (10 mL) was added HCl in 1,4-dioxane (4 M solution, 10 mL) dropwise at 0° C. The resulting mixture was stirred for 1 h at room temperature and was then concentrated under reduced pressure to give 3-(2-(5-(4-cyclopropylpiperazin-1-yl)-2-((S)-1-methoxyethyl)pyridin-3-yl)-1-ethyl-5-(4,4,5,5-tetramethyl-1,3,2-dioxaborolan-2-yl)-1H-indol-3-yl)-2,2-dimethylpropyl-(S)-hexahydropyridazine-3-carboxylate dihydrochloride (980 mg, crude), which was used without further purification. LCMS (ESI) m/z: [M+H] calcd for C₄₁H₆₁BN₆O₅: 729.5; found 729.4.

Step 12. To a stirred solution of 3-(2-(5-(4-cyclopropylpiperazin-1-yl)-2-((S)-1-methoxyethyl)pyridin-3-yl)-1-ethyl-5-(4,4,5,5-tetramethyl-1,3,2-dioxaborolan-2-yl)-1H-indol-3-yl)-2,2-dimethylpropyl (S)-hexahydropyridazine-3-carboxylate (200 mg, crude), DIPEA (355 mg, 2.74 mmol) and (1²R,1⁵S,6S,9S)-4⁵-bromo-1⁵-(dimethylcarbamoyl)-9-isopropyl-10-methyl-8,11-dioxo-7,10-diaza-1(1,2)-pyrrolidina-4(1,3)-benzenacycloundecaphane-6-carboxylic acid (227 mg, 0.411 mmol) in DMF (3 mL) was added HATU (156 mg, 0.411 mmol) at 0° C. The resulting mixture was stirred for 16 h at room temperature and was then quenched by the addition of H₂O (15 mL) at 0° C. The resulting mixture was extracted with EtOAc (3×30 mL) and the combined organic extracts were washed with brine (3×20 mL), dried over Na₂SO₄, filtered, and concentrated under reduced pressure. The residue was purified by normal phase prep-TLC to afford 3-(2-(5-(4-cyclopropylpiperazin-1-yl)-2-((S)-1-methoxyethyl)pyridin-3-yl)-1-ethyl-5-(4,4,5,5-tetramethyl-1,3,2-dioxaborolan-2-yl)-1H-indol-3-yl)-2,2-dimethylpropyl-(S)-1-((1²R,1⁵S,6S,9S)-4⁵-bromo-1⁵-(dimethylcarbamoyl)-9-isopropyl-10-methyl-8,11-dioxo-7,10-diaza-1(1,2)-pyrrolidina-4(1,3)-benzenacycloundecaphane-6-carbonyl)hexahydropyridazine-3-carboxylate (266 mg, 51% yield) as a yellow solid. LCMS (ESI) m/z: [M+H] calcd for C₆₆H₉₄BBrN₁₀O₉: 1261.7; found 1261.6.

Step 13. To a solution of 3-(2-(5-(4-cyclopropylpiperazin-1-yl)-2-((S)-1-methoxyethyl)pyridin-3-yl)-1-ethyl-5-(4,4,5,5-tetramethyl-1,3,2-dioxaborolan-2-yl)-1H-indol-3-yl)-2,2-dimethylpropyl-(S)-1-((1²R,1⁵S,6S,9S)-4⁵-bromo-1⁵-(dimethylcarbamoyl)-9-isopropyl-10-methyl-8,11-dioxo-7,10-diaza-1(1,2)-pyrrolidina-4(1,3)-benzenacycloundecaphane-6-carbonyl)hexahydropyridazine-3-carboxylate (285 mg, 0.226 mmol) in dioxane (1.5 mL), H₂O (1.5 mL), and toluene (4.5 mL) at room temperature were added K₃PO₄ (95.9 mg, 0.452 mmol) and Pd(dtbpf)Cl₂ (29.4 mg, 0.045 mmol). The resulting mixture was stirred for 2 h at 70° C. under an atmosphere of N₂ and was then filtered. The filter cake was washed with EtOAc (3×15 mL) and the filtrate was treated with H₂O (20 mL, extracted with EtOAc (3×15 m), and the combined organic extracts were dried over Na₂SO₄, filtered, and concentrated under reduced pressure.

The crude residue was purified by reversed phase chromatography to afford (9S,15S,18S,20aR,23S)-3-(5-(4-cyclopropylpiperazin-1-yl)-2-((S)-1-methoxyethyl)pyridin-3-yl)-2-ethyl-18-isopropyl-N,N,5,5,19-pentamethyl-8,14,17,20-tetraoxo-2,4,5,6,9,10,11,12,15,16,17,18,19,20,20a,21,22,23,25,26-icosahydro-8H,14H-9,13-epimino-1,32-etheno-15,29-methano-27,31-(metheno)dipyrrolo[2,1-o:3′,4′-z][1]oxa[7,10,13,16]tetraazacyclotriacontine-23-carboxamide (30.6 mg, 13% yield) as a white solid. LCMS (ESI) m/z: [M+H] calcd for C₆₀H₈₂N₁₀O₇: 1055.6; found 1055.6.5H NMR (400 MHz, DMSO-d₆) δ8.39 (br s, 1H), 8.20 (d, J=7.1 Hz, 1H), 7.96 (s, 1H), 7.67 (d, J=8.6 Hz, 1H), 7.59 (s, 1H), 7.52-7.40 (m, 2H), 7.15 (s, 1H), 7.03 (s, 1H), 5.34-5.05 (m, 2H), 4.68 (d, J=10.8 Hz, 1H), 4.26-3.95 (m, 5H), 3.69-3.58 (m, 5H), 3.22-3.10 (m, 10H), 3.01 (s, 4H), 2.79-2.68 (m, 7H), 2.44 (br s, 5H), 2.21-92.03 (m, 2H), 2.01-1.70 (m, 6H), 1.66-1.37 (m, 3H), 1.33-1.10 (m, 4H), 0.92 (s, J=6.9 Hz, 3H), 0.79 (d, J=6.7 Hz, 4H), 0.72-0.57 (m, 5H), 0.48 (s, 3H), 0.37 (d, J=5.6 Hz, 2H), 0.27 (s, 2H).

TABLE 3
Exemplary Compounds Prepared by
Methods of the Present Invention

| | | LCMS |
|---|---|---|
| | LCMS | (ESI): |
| | (ESI): m/z | m/z |
| | [M + H] | [M + H] |
| Ex # | Calculated | Found |
| A1 | 916.5 | 916.4 |
| A2 | 999.6 | 999.5 |
| A3 | 914.5 | 914.6 |
| A4 | 1002.6 | 1002.4 |
| A5 | 1000.6 | 1000.7 |
| A6 | 918.5 | 918.4 |
| A7 | 918.5 | 918.5 |
| A8 | 918.5 | 918.4 |
| A9 | 973.5 | 973.3 |
| A10 | 973.5 | 973.5 |
| A11 | 973.5 | 973.5 |
| A12 | 942.5 | 942.5 |
| A13 | 942.5 | 942.5 |
| A14 | 956.5 | 956.5 |
| A15 | 956.5 | 956.5 |
| A16 | 930.5 | 930.5 |
| A17 | 930.5 | 930.5 |
| A18 | 930.5 | 930.5 |
| A19 | 930.5 | 930.5 |
| A20 | 950.5 | 950.5 |
| A21 | 950.5 | 950.4 |
| A22 | 1014.6 | 1014.6 |
| A23 | 904.5 | 904.5 |
| A24 | 1014.6 | 1014.7 |
| A25 | 944.5 | 945.2 |
| A26 | 944.5 | 944.8 |
| A27 | 990.6 | 990.7 |
| A28 | 920.5 | 920.5 |
| A29 | 933.5 | 933.5 |
| A30 | 932.5 | 932.5 |
| A31 | 967.5 | 967.3 |
| A32 | 934.5 | 934.4 |
| A33 | 1002.6 | 1002.5 |
| A34 | 973.5 | 973.5 |
| A35 | 930.5 | 930.5 |
| A36 | 987.6 | 987.5 |
| A37 | 947.5 | 947.5 |
| A38 | 975.6 | 975.4 |
| A39 | 967.5 | 967.4 |
| A40 | 967.5 | 967.3 |
| A41 | 960.5 | 960.5 |
| A42 | 1002.6 | 1002.5 |
| A43 | 892.5 | 892.8 |
| A44 | 1032.6 | 1032.7 |
| A45 | 1016.6 | 1016.7 |
| A46 | 1044.6 | 1044.8 |
| A47 | 1071.5 | 1071.3 |
| A48 | 1028.6 | 1028.6 |
| A49 | 1057.6 | 1057.5 |
| A50 | 1072.6 | 1072.7 |
| A51 | 1016.6 | 1016.5 |
| A52 | 1068.6 | 1068.7 |
| A53 | 1073.6 | 1073.7 |
| A54 | 1075.6 | 1075.6 |
| A55 | 1055.6 | 1055.4 |
| A56 | 1087.6 | 1087.4 |
| A57 | 1058.6 | 1058.8 |
| A58 | 1069.6 | 1069.8 |
| A59 | 1065.6 | 1065.3 |
| A60 | 1065.6 | 1065.9 |
| A61 | 1065.6 | 1065.4 |
| A62 | 975.5 | 975.5 |
| A63 | 989.5 | 989.3 |
| A64 | 973.5 | 973.3 |
| A65 | 971.5 | 971.5 |
| A66 | 989.5 | 989.3 |
| A67 | 944.5 | 944.5 |
| A68 | 970.5 | 970.4 |
| A69 | 1031.6 | 1031.9 |
| A70 | 976.5 | 976.5 |
| A71 | 960.5 | 960.4 |
| A72 | 1058.6 | 1058.5 |
| A73 | 1071.5 | 1071.8 |
| A74 | 1032.6 | 1032.7 |
| A75 | 1018.6 | 1018.5 |
| A76 | 1060.6 | 1061.3 |
| A77 | 1078.6 | 1078.9 |
| A78 | 1054.6 | 1055.3 |
| A79 | 1043.6 | 1044.1 |
| A80 | 1042.6 | 1042.6 |
| A81 | 1034.6 | 1035.0 |
| A82 | 1058.6 | 1059.0 |
| A83 | 1058.6 | 1059.5 |
| A84 | 1057.6 | 1057.9 |
| A85 | 1042.6 | 1043.2 |
| A86 | 1032.6 | 1033.2 |
| A87 | 1058.6 | 1060.0 |
| A88 | 1060.6 | 1061.5 |
| A89 | 1030.6 | 1031.1 |
| A90 | 1042.6 | 1043.2 |
| A91 | 1030.6 | 1031.0 |
| A92 | 1042.6 | 1042.9 |
| A93 | 1078.6 | 1079.0 |
| A94 | 1087.6 | 1087.5 |
| A95 | 1072.6 | 1072.9 |
| A96 | 1073.6 | 1073.6 |
| A97 | 1044.6 | 1044.5 |
| A98 | 1044.6 | 1045.1 |
| A99 | 1055.6 | 1056.1 |
| A100 | 1046.6 | 1047.9 |
| A101 | 1046.6 | 1047.1 |
| A102 | 1052.6 | 1053.0 |
| A103 | 1044.6 | 1044.9 |
| A104 | 1068.6 | 1068.9 |
| A105 | 1084.6 | 1085.0 |
| A106 | 1060.6 | 1061.0 |
| A107 | 1070.6 | 1070.9 |
| A108 | 1052.6 | 1053.0 |
| A109 | 1046.6 | 1047.0 |
| A110 | 1060.6 | 1061.0 |
| A111 | 1056.6 | 1057.1 |
| A112 | 1092.6 | 1093.1 |
| A113 | 1078.6 | 1079.0 |
| A114 | 1074.6 | 1075.1 |
| A115 | 1060.6 | 1061.0 |
| A116 | 1072.6 | 1073.1 |
| A117 | 1070.6 | 1071.0 |
| A118 | 1048.6 | 1049.1 |
| A119 | 1046.6 | 1047.0 |
| A120 | 1078.6 | 1079.0 |
| A121 | 1053.6 | 1053.9 |
| A122 | 1087.6 | 1088.0 |
| A123 | 1087.6 | 1087.9 |
| A124 | 1120.6 | 1120.8 |
| A125 | 1073.6 | 1073.7 |
| A126 | 1043.5 | 1043.4 |
| A127 | 1028.6 | 1028.5 |
| A128 | 1054.6 | 1054.6 |
| A129 | 1070.6 | 1070.5 |
| A130 | 1056.6 | 1056.4 |
| A131 | 1058.6 | 1058.6 |
| A132 | 1097.6 | 1097.5 |
| A133 | 1060.6 | 1060.6 |
| A134 | 1078.6 | 1078.6 |
| A135 | 1056.6 | 1055.6 |
| A136 | 1058.6 | 1058.5 |
| A137 | 1097.6 | 1097.4 |
| A138 | 1064.6 | 1064.4 |
| A139 | 1056.6 | 1056.3 |
| A140 | 1016.6 | 1016.9 |
| A141 | 1074.6 | 1074.5 |
| A142 | 1068.6 | 1068.5 |
| A143 | 1082.6 | 1082.7 |
| A144 | 1081.6 | 1081.6 |
| A145 | 1084.6 | 1084.5 |
| A146 | 1072.6 | 1072.8 |
| A147 | 1079.6 | 1079.4 |
| A148 | 1098.6 | 1098.5 |
| A149 | 1015.6 | 1015.8 |
| A150 | 1068.6 | 1068.9 |
| A151 | 1096.6 | 1096.9 |
| A152 | 1104.6 | 1104.7 |
| A153 | 1122.6 | 1122.4 |
| A154 | 1086.6 | 1086.8 |
| A155 | 1088.6 | 1088.6 |
| A156 | 1070.6 | 1070.3 |
| A157 | 1136.6 | 1136.4 |
| A158 | 1078.6 | 1078.5 |
| A159 | 1095.6 | 1095.8 |
| A160 | 1041.6 | 1041.9 |
| A161 | 1098.6 | 1098.8 |
| A162 | 1114.6 | 1114.5 |
| A163 | 1135.6 | 1135.9 |
| A164 | 1140.6 | 1140.9 |
| A165 | 1138.6 | 1138.6 |
| A166 | 1097.6 | 1097.7 |
| A167 | 1113.6 | 1113.7 |
| A168 | 1121.6 | 1121.6 |
| A169 | 1127.6 | 1127.4 |
| A170 | 1125.6 | 1125.3 |
| A171 | 1139.6 | 1139.3 |
| A172 | 1157.6 | 1157.6 |
| A173 | 1137.6 | 1137.6 |
| A174 | 1134.6 | 1134.9 |
| A175 | 1077.6 | 1077.6 |
| A176 | 1073.6 | 1073.5 |
| A177 | 1083.6 | 1083.6 |
| A178 | 1058.6 | 1058.5 |
| A179 | 1132.6 | 1132.7 |
| A180 | 1043.6 | 1043.5 |
| A181 | 1059.6 | 1059.5 |
| A182 | 1067.6 | 1067.5 |
| A183 | 1071.6 | 1071.9 |
| A184 | 1085.6 | 1085.9 |
| A185 | 1103.6 | 1103.8 |
| A186 | 1080.6 | 1080.5 |
| A187 | 1091.6 | 1091.6 |
| A188 | 1076.6 | 1076.5 |
| A189 | 1072.6 | 1072.4 |
| A190 | 1070.6 | 1070.9 |
| A191 | 1088.6 | 1088.9 |
| A192 | 1086.6 | 1086.4 |
| A193 | 1084.6 | 1084.5 |
| A194 | 1040.6 | 1040.5 |
| A195 | 1057.6 | 1057.9 |
| A196 | 1095.6 | 1095.4 |
| A197 | 1093.6 | 1093.9 |
| A198 | 1089.6 | 1090.0 |
| A199 | 1059.6 | 1060.0 |
| A200 | 1093.6 | 1094.0 |
| A201 | 1086.6 | 1087.1 |
| A202 | 1118.6 | 1119.1 |
| A203 | 1098.6 | 1099.3 |
| A204 | 1094.6 | 1095.0 |
| A205 | 1070.6 | 1071.2 |
| A206 | 1092.6 | 1094.2 |
| A207 | 1072.6 | 1073.2 |
| A208 | 1084.6 | 1085.1 |
| A209 | 1104.6 | 1105.0 |
| A210 | 1100.6 | 1100.7 |
| A211 | 1106.6 | 1107.2 |
| A212 | 1100.6 | 1100.7 |
| A213 | 1118.6 | 1119.1 |
| A214 | 1084.6 | 1085.2 |
| A215 | 1084.6 | 1085.1 |
| A216 | 1084.6 | 1085.2 |
| A217 | 1086.6 | 1087.1 |
| A218 | 1072.6 | 1073.1 |
| A219 | 1072.6 | 1073.1 |
| A220 | 1068.6 | 1069.0 |
| A221 | 1070.6 | 1071.2 |
| A222 | 1082.6 | 1083.0 |
| A223 | 1086.6 | 1087.1 |
| A224 | 1104.6 | 1105.1 |
| A225 | 1110.6 | 1111.0 |
| A226 | 1068.6 | 1069.2 |
| A227 | 1068.6 | 1069.2 |
| A228 | 1098.6 | 1099.2 |
| A229 | 1108.7 | 1109.8 |
| A230 | 1096.7 | 1097.1 |
| A231 | 1114.6 | 1115.1 |
| A232 | 1122.7 | 1123.0 |
| A233 | 1130.6 | 1131.0 |
| A234 | 1096.7 | 1097.1 |
| A235 | 1080.6 | 1081.0 |
| A236 | 1094.6 | 1095.1 |
| A237 | 1108.7 | 1109.0 |
| A238 | 1144.6 | 1145.0 |
| A239 | 1162.6 | 1163.0 |
| A240 | 1130.6 | 1131.0 |
| A241 | 1056.6 | 1056.9 |
| A242 | 1088.6 | 1089.0 |
| A243 | 1080.6 | 1081.0 |
| A244 | 1068.6 | 1069.0 |
| A245 | 1080.6 | 1080.9 |
| A246 | 1094.6 | 1095.1 |
| A247 | 1122.6 | 1123.0 |
| A248 | 1094.6 | 1095.0 |
| A249 | 1126.6 | 1127.0 |
| A250 | 1148.6 | 1149.0 |
| A251 | 1094.6 | 1095.0 |
| A252 | 1112.6 | 1113.1 |
| A253 | 1094.6 | 1094.5 |
| A254 | 1086.6 | 1086.6 |
| A255 | 1083.6 | 1084.0 |
| A256 | 1102.6 | 1102.7 |
| A257 | 1124.6 | 1124.6 |
| A258 | 1106.6 | 1106.9 |
| A259 | 1056.6 | 1056.6 |
| A260 | 1072.6 | 1072.6 |
| A261 | 1088.6 | 1088.6 |
| A262 | 1083.6 | 1084.2 |
| A263 | 1079.6 | 1079.7 |
| A264 | 1101.6 | 1101.6 |
| A265 | 1097.6 | 1098.5 |
| A266 | 1067.6 | 1067.6 |
| A267 | 1090.6 | 1091.2 |
| A268 | 1104.6 | 1105.1 |
| A269 | 1120.6 | 1120.9 |
| A270 | 1118.6 | 1119.2 |
| A271 | 1106.6 | 1106.9 |
| A272 | 1112.6 | 1112.7 |
| A273 | 1100.6 | 1100.7 |
| A274 | 1131.6 | 1131.8 |
| A275 | 1084.6 | 1084.6 |
| A276 | 1090.6 | 1089.6 |
| A277 | 1114.6 | 1114.8 |
| A278 | 1060.6 | 1060.6 |
| A279 | 1074.6 | 1074.6 |
| A280 | 1084.6 | 1084.6 |
| A281 | 1080.6 | 1080.7 |
| A282 | 1074.6 | 1074.7 |
| A283 | 1050.6 | 1050.6 |
| A284 | 1102.6 | 1102.8 |
| A285 | 1098.6 | 1098.7 |
| A286 | 1092.6 | 1092.8 |
| A287 | 1068.6 | 1068.7 |
| A288 | 1094.6 | 1094.6 |
| A289 | 1090.6 | 1090.6 |
| A290 | 1084.6 | 1084.7 |
| A291 | 1060.6 | 1060.6 |
| A292 | 1049.6 | 1049.6 |
| A293 | 1091.6 | 1091.7 |
| A295 | 1126.6 | 1127.6 |
| A296 | 1086.6 | 1086.6 |
| A297 | 1088.6 | 1088.7 |
| A298 | 1088.6 | 1088.7 |
| A299 | 1104.6 | 1105.0 |
| A300 | 1085.6 | 1086.1 |
| A301 | 1082.6 | 1083.0 |
| A302 | 1104.6 | 1105.0 |
| A303 | 1118.6 | 1119.1 |
| A304 | 1132.6 | 1133.1 |
| A305 | 1118.6 | 1119.0 |
| A306 | 1140.6 | 1141.0 |
| A307 | 1000.6 | 1000.5 |
| A308 | 1091.6 | 1091.6 |
| A309 | 1073.6 | 1073.8 |
| A310 | 1075.6 | 1075.4 |
| A311 | 1140.6 | 1141.0 |
| A312 | 1134.6 | 1134.9 |
| A313 | 1140.6 | 1140.9 |
| A314 | 1146.6 | 1147.0 |
| A315 | 1128.6 | 1128.8 |
| A316 | 1082.5 | 1082.8 |
| A317 | 1114.6 | 1114.8 |
| A318 | 1109.6 | 1109.8 |
| A319 | 1109.6 | 1109.7 |
| A320 | 1055.6 | 1055.6 |
| A321 | 1055.6 | 1055.8 |
| A322 | 1078.6 | 1078.6 |
| A323 | 1100.6 | 1100.8 |
| A324 | 1143.6 | 1143.7 |
| A325 | 1147.6 | 1147.7 |
| A326 | 1074.6 | 1074.8 |
| A327 | 1092.6 | 1092.5 |
| A328 | 1092.6 | 1092.5 |
| A329 | 1086.6 | 1086.7 |
| A330 | 1137.6 | 1137.7 |
| A331 | 1099.6 | 1099.7 |
| A332 | 1078.6 | 1078.5 |
| A333 | 1063.6 | 1063.6 |
| A334 | 1045.6 | 1045.6 |
| A335 | 1041.6 | 1041.6 |
| A336 | 1072.6 | 1072.7 |
| A337 | 1133.6 | 1133.6 |
| A338 | 1145.6 | 1145.6 |
| A340 | 1082.6 | 1082.8 |
| A341 | 1063.6 | 1063.5 |
| A342 | 1063.6 | 1063.5 |
| A343 | 1151.6 | 1151.7 |
| A344 | 1092.6 | 1092.6 |
| A345 | 1101.6 | 1101.7 |
| A346 | 1074.6 | 1074.6 |
| A347 | 1140.6 | 1140.7 |
| A348 | 1060.6 | 1060.7 |
| A349 | 1128.6 | 1128.5 |
| A350 | 1128.6 | 1128.5 |
| A351 | 1087.6 | 1087.5 |
| A352 | 1132.6 | 1132.7 |
| A353 | 1060.6 | 1060.7 |
| A354 | 1057.5 | 1057.5 |
| A355 | 507.8* | 507.9* |
| A356 | 546.3* | 545.9* |
| A357 | 568.3* | 568.8* |
| A358 | 1069.6 | 1069.4 |
| A359 | 1150.6 | 1150.7 |
| A360 | 1104.6 | 1104.5 |
| A361 | 532.3* | 532.0* |
| A362 | 1088.6 | 1088.5 |
| A363 | 1073.6 | 1073.6 |
| A364 | 1146.6 | 1146.5 |
| A365 | 1043.5 | 1043.5 |
| A366 | 1104.6 | 1104.5 |
| A367 | 1157.6 | 1157.5 |
| A368 | 1173.6 | 1173.5 |
| A369 | 1132.6 | 1132.6 |
| A370 | 1176.6 | 1176.7 |
| A371 | 1110.6 | 1110.6 |
| A372 | 1110.6 | 1110.6 |
| A373 | 1142.6 | 1142.6 |
| A374 | 1069.6 | 1069.4 |
| A375 | 1061.6 | 1061.5 |
| A376 | 1157.6 | 1157.5 |
| A377 | 1163.6 | 1163.4 |
| A378 | 1163.6 | 1163.4 |
| A379 | 1174.6 | 1174.7 |
| A380 | 1174.6 | 1174.6 |
| A381 | 1144.6 | 1144.6 |
| A382 | 1148.6 | 1148.7 |
| A383 | 1138.6 | 1138.6 |
| A384 | 1062.6 | 1062.6 |
| A385 | 1173.6 | 1173.6 |
| A386 | 1181.6 | 1181.5 |
| A387 | 1181.6 | 1181.5 |
| A388 | 1176.6 | 1176.7 |
| A389 | 1176.6 | 1176.7 |
| A390 | 1086.6 | 1086.7 |
| A391 | 1060.6 | 1060.6 |
| A392 | 1150.6 | 1150.7 |
| A393 | 1076.6 | 1076.5 |
| A394 | 1129.5 | 1129.6 |
| A395 | 1130.5 | 1130.5 |
| A396 | 1150.6 | 1150.8 |
| A397 | 1150.6 | 1150.7 |
| A398 | 1060.6 | 1060.6 |
| A399 | 1103.6 | 1103.6 |
| A400 | 1150.6 | 1148.6 |
| A401 | 1150.6 | 1150.8 |
| A402 | 1053.6 | 1053.6 |
| A403 | 1069.6 | 1069.6 |
| A404 | 1053.6 | 1053.6 |
| A405 | 950.5 | 950.5 |
| A406 | 1074.6 | 1074.8 |
| A407 | 1074.6 | 1074.6 |
| A408 | 1088.6 | 1088.9 |
| A409 | 1088.6 | 1088.8 |
| A410 | 1079.6 | 1079.6 |
| A411 | 1075.6 | 1075.6 |
| A412 | 1075.6 | 1075.6 |
| A413 | 1037.6 | 1037.5 |
| A415 | 1063.6 | 1062.6 |
| A416 | 1069.6 | 1069.7 |
| A418 | 1100.6 | 1100.8 |
| A419 | 1100.6 | 1100.7 |
| A420 | 1163.6 | 1163.8 |
| A421 | 1163.6 | 1163.7 |
| A423 | 1034.6 | 1034.6 |
| A424 | 1041.6 | 1041.6 |
| A425 | 1097.6 | 1097.9 |
| A426 | 1028.6 | 1028.5 |
| A427 | 1062.6 | 1062.6 |
| A428 | 1016.6 | 1016.6 |
| A429 | 1016.6 | 1016.6 |
| A432 | 1055.6 | 1055.6 |
| A433 | 1060.6 | 1060.6 |
| A434 | 1113.6 | 1113.6 |
| A442 | 1062.6 | 1062.6 |
| A443 | 1039.6 | 1039.6 |
| A444 | 986.6 | 986.6 |
| A448 | 1136.6 | 1136.6 |
| A450 | 1123.6 | 1123.6 |

Note:
values may differ slightly from values found elsewhere in this application due to different measurements and rounding.

TABLE 4
Exemplary Compounds Prepared by
Methods of the Present Invention

| | LCMS (ESI): |
|---|---|
| | m/z [M + H] |
| Ex # | Found |
| B1 | 1069.7 |
| B2 | 1109.7 |
| B3 | 1097.7 |
| B4 | 1097.6 |
| B5 | 1097.6 |
| B6 | 1043.5 |
| B7 | 1043.5 |
| B8 | 1055.5 |
| B9 | 1029.7 |
| B10 | 1029.7 |
| B11 | 1097.7 |
| B12 | 1111.6 |
| B13 | 1097.6 |
| B14 | 1099.6 |
| B15 | 1071.6 |
| B16 | 1099.6 |
| B17 | 1085.5 |
| B18 | 1085.5 |
| B19 | 1085.5 |
| B20 | 1059.7 |
| B21 | 1099.6 |
| B22 | 1085.6 |
| B23 | 1087.6 |
| B24 | 1059.5 |
| B25 | 1078.6 |
| B26 | 1000.6 |
| B27 | 1097.6 |
| B28 | 1110.6 |
| B29 | 956.5 |
| B30 | 860.5 |
| B31 | 1002.5 |
| B32 | 860.5 |
| B33 | 861.4 |
| B34 | 862.5 |
| B35 | 862.4 |

### Biological Assays

Disruption of B-Raf Ras-Binding Domain (BRAF^{RBD}) Interaction with K-Ras by Compounds of the Invention (Also Called a FRET Assay or an MOA Assay)

The purpose of this biochemical assay is to measure the ability of test compounds to facilitate ternary complex formation between a nucleotide-loaded K-Ras isoform and cyclophilin A; the resulting ternary complex disrupts binding to a BRAF^{RBD} Construct, inhibiting K-Ras signaling through a RAF effector. Data is reported as IC50 values. Other Ras variants may be used.

In assay buffer containing 25 mM HEPES pH 7.3, 0.002% Tween20, 0.1% BSA, 100 mM NaCl and 5 mM MgCl₂, tagless cyclophilin A, His6-K-Ras-GMPPNP, and GST-BRAF^{RBD} are combined in a 384-well assay plate at final concentrations of 25 μM, 12.5 nM and 50 nM, respectively. Compound is present in plate wells as a 10-point 3-fold dilution series starting at a final concentration of 30 μM. After incubation at 25° C. for 3 hours, a mixture of Anti-His Eu—W1024 and anti-GST allophycocyanin is then added to assay sample wells at final concentrations of 10 nM and 50 nM, respectively, and the reaction incubated for an additional 1.5 hours. TR-FRET signal is read on a microplate reader (Ex 320 nm, Em 665/615 nm). Compounds that facilitate disruption of a K-Ras:RAF complex are identified as those eliciting a decrease in the TR-FRET ratio relative to DMSO control wells.

Each of Examples A1-A450 exhibited an IC50 of less than 2 μM in at least one of the following: K-Ras Q61H, G12C, G12D, G12R, G12S, G12V, G12A, G13C, G13D and wild-type; N-Ras Q61K, Q61R, Q61L, G12C and wild-type; and H-Ras G13R and WT.

While the invention has been described in connection with specific embodiments thereof, it will be understood that it is capable of further modifications and this application is intended to cover any variations, uses, or adaptations of the invention following, in general, the principles of the invention and including such departures from the present disclosure come within known or customary practice within the art to which the invention pertains and may be applied to the essential features set forth herein.

All publications, patents and patent applications are herein incorporated by reference in their entirety to the same extent as if each individual publication, patent, or patent application was specifically and individually indicated to be incorporated by reference in its entirety.

### Enumerated Embodiments

E1. A compound, or pharmaceutically acceptable salt thereof, having the structure of Formula Ia-2:

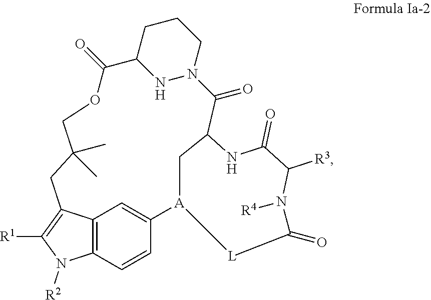

wherein
A is optionally substituted 3- to 6-membered heterocycloalkylene, optionally substituted 3- to 6-membered cycloalkylene, optionally substituted 6-membered arylene, or optionally substituted 5- to 10-membered heteroarylene;
L is a linker;
R¹ is optionally substituted 5- to 10-membered heteroaryl;
R² is optionally substituted C₁-C₆ alkyl;
R³ is optionally substituted C₁-C₆ alkyl, optionally substituted C₁-C₃ heteroalkyl, or optionally substituted 3- to 6-membered cycloalkyl; and
R⁴ is hydrogen or optionally substituted C₁-C₆ alkyl.

E2. The compound of embodiment 1, or a pharmaceutically acceptable salt thereof, wherein the compound has the structure of Formula IIa-2:

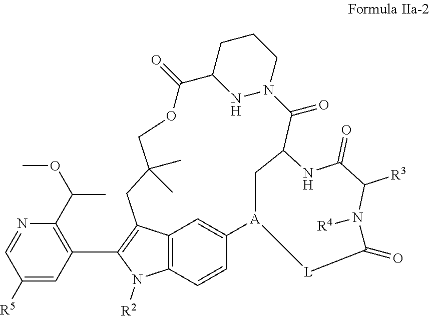

wherein
R⁵ is hydrogen, optionally substituted 3- to 10-membered heterocycloalkyl, —OR^{5a}, or optionally substituted C₁-C₆ heteroalkyl; and
R^{5a} is optionally substituted C₁-C₆ alkyl or optionally substituted 5- to 10-membered heteroaryl.

E3. The compound of embodiment 2, or a pharmaceutically acceptable salt thereof, wherein the compound has the structure of Formula IIIa-2:

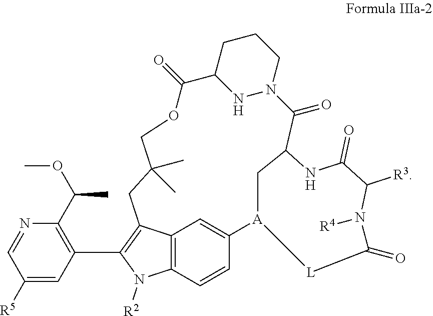

E4. The compound of any one of embodiments 1 to 3, or a pharmaceutically acceptable salt thereof, wherein
L has the structure of Formula IV:

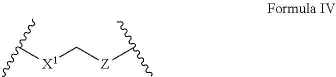

X¹ is O or CH₂ and is attached to ring A; and
Z is optionally substituted 3- to 6-membered heterocycloalkylene, optionally substituted C₁-C₆ alkylene, or optionally substituted C₁-C₆ heteroalkylene.

E5. The compound of embodiment 2, or a pharmaceutically acceptable salt thereof, wherein the compound has the structure of Formula Va-2:

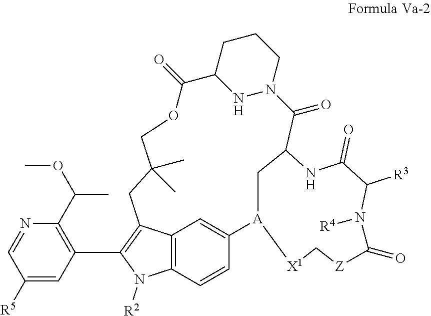

wherein
X¹ is O or CH₂; and
Z is optionally substituted 3- to 6-membered heterocycloalkylene, optionally substituted C₁-C₆ alkylene, or optionally substituted C₁-C₆ heteroalkylene.

E6. The compound of embodiment 4 or 5, or a pharmaceutically acceptable salt thereof, wherein X¹ is O.

E7. The compound of any one of embodiments 4 to 6, or a pharmaceutically acceptable salt thereof, wherein Z is optionally substituted 3- to 6-membered heterocycloalkylene.

E8. The compound of embodiment 7, or a pharmaceutically acceptable salt thereof, wherein Z is optionally substituted 5-membered heterocycloalkylene.

E9. The compound of embodiment 8, or a pharmaceutically acceptable salt thereof, wherein Z is optionally substituted pyrollidine-diyl.

E10. The compound of any one of embodiments 1 to 3, or a pharmaceutically acceptable salt thereof, wherein
L has the structure of Formula VI:

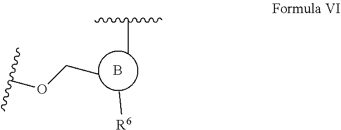

B is an optionally substituted 3- to 6-membered heterocycloalkylene;
R⁶ is hydrogen, optionally substituted C₁-C₆ alkyl, optionally substituted C₁-C₆ heteroalkyl, optionally substituted optionally substituted 3- to 6-membered heterocyclyl, optionally substituted 3- to 6-membered cycloalkyl, optionally substituted 5- to 10-membered heteroaryl, optionally substituted C₆-C₁₀ aryl,

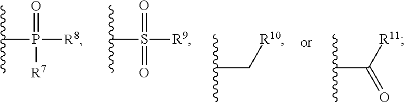

R⁷ and R⁸ are each, independently, H or optionally substituted C₁-C₆ alkyl;
R⁹ is optionally substituted C₁-C₆ alkyl, optionally substituted C₂-C₆ alkenyl, optionally substituted 3- to 6-membered cycloalkyl, or optionally substituted 3- to 6-membered heterocyclyl;
R¹⁰ is optionally substituted C₁-C₆ alkyl, optionally substituted C₂-C₆ alkenyl, optionally substituted C₁-C₆ heteroalkyl, optionally substituted C₃-C₁₀ cycloalkyl, optionally substituted 3- to 6-membered heterocyclyl, optionally substituted 5- to 10-membered heteroaryl, or optionally substituted C₆-C₁₀ aryl; and
R¹¹ is hydrogen, optionally substituted C₁-C₆ alkyl, optionally substituted C₁-C₆ heteroalkyl, optionally substituted C₂-C₆ alkenyl, optionally substituted C₂-C₆ heteroalkenyl, optionally substituted C₂-C₆ alkynyl, optionally substituted C₂-C₆ heteroalkynyl, optionally substituted C₃-C₁₀ cycloalkyl, optionally substituted 3- to 10-membered heterocyclyl, optionally substituted C₃-C₁₀ cycloalkenyl, optionally substituted 3- to 10-membered heterocycloalkenyl, optionally substituted C₆-C₁₀ aryl, or optionally substituted 5- to 10-membered heteroaryl.

E11. The compound of embodiment 10, or a pharmaceutically acceptable salt thereof, wherein L has the structure of Formula IVa:

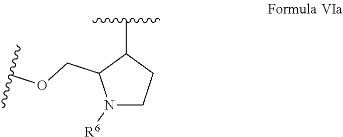

E12. The compound of embodiment 2, or a pharmaceutically acceptable salt thereof, wherein the compound has the structure of Formula IIc:

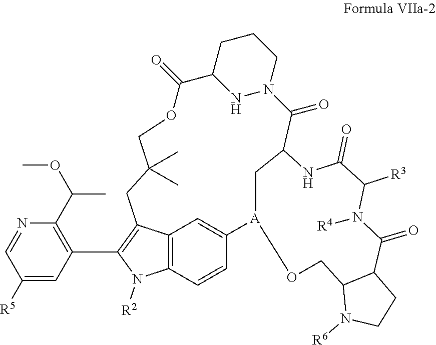

wherein
R⁶ is hydrogen, optionally substituted C₁-C₆ alkyl, optionally substituted C₁-C₆ heteroalkyl, optionally substituted 3- to 6-membered heterocyclyl, optionally substituted 3- to 6-membered cycloalkyl, optionally substituted 5- to 10-membered heteroaryl, optionally substituted C₆-C₁₀ aryl,

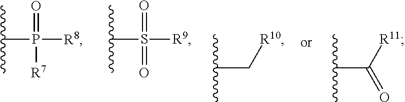

R⁷ and R⁸ are each, independently, H or optionally substituted C₁-C₆ alkyl;
R⁹ is optionally substituted C₁-C₆ alkyl, optionally substituted C₂-C₆ alkenyl, optionally substituted 3- to 6-membered cycloalkyl, or optionally substituted 3- to 6-membered heterocyclyl;
R¹⁰ is optionally substituted C₁-C₆ alkyl, optionally substituted C₂-C₆ alkenyl, optionally substituted C₁-C₆ heteroalkyl, optionally substituted C₃-C₁₀ cycloalkyl, optionally substituted 3- to 6-membered heterocyclyl, optionally substituted 5- to 10-membered heteroaryl, or optionally substituted C₆-C₁₀ aryl; and
R¹¹ is hydrogen, optionally substituted C₁-C₆ alkyl, optionally substituted C₁-C₆ heteroalkyl, optionally substituted C₂-C₆ alkenyl, optionally substituted C₂-C₆ heteroalkenyl, optionally substituted C₂-C₆ alkynyl, optionally substituted C₂-C₆ heteroalkynyl, optionally substituted C₃-C₁₀ cycloalkyl, optionally substituted 3- to 10-membered heterocyclyl, optionally substituted C₃-C₁₀ cycloalkenyl, optionally substituted 3- to 10-membered heterocycloalkenyl, optionally substituted C₆-C₁₀ aryl, or optionally substituted 5- to 10-membered heteroaryl.

E13. A compound having the structure of Formula Ia or Formula Ib:

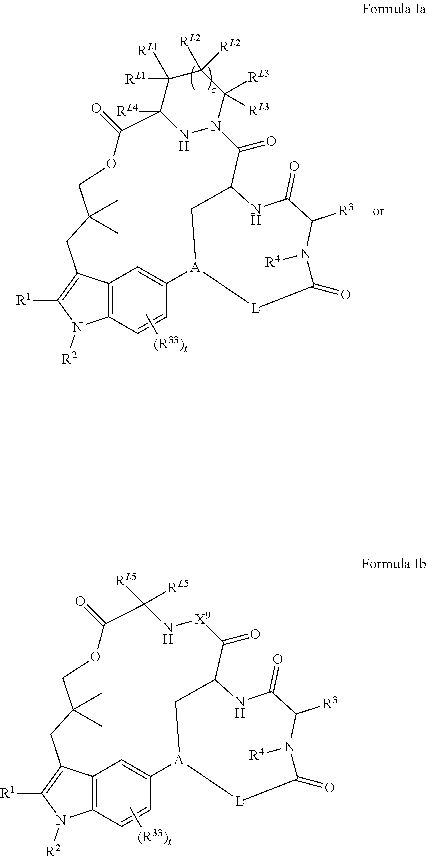

or a pharmaceutically acceptable salt thereof, wherein
A is optionally substituted 3- to 6-membered heterocycloalkylene, optionally substituted 3- to 6-membered cycloalkylene, optionally substituted 6-membered arylene, or optionally substituted 5- to 10-membered heteroarylene;
L is a linker;
R¹ is optionally substituted 5- to 10-membered heteroaryl;
R² is optionally substituted C₁-C₆ alkyl;
R³ is optionally substituted C₁-C₆ alkyl, optionally substituted C₁-C₃ heteroalkyl, or optionally substituted 3- to 6-membered cycloalkyl;
R⁴ is hydrogen or optionally substituted C₁-C₆ alkyl;
each R³³ is, independently, halogen, optionally substituted C₁-C₃ alkyl, optionally substituted C₁-C₃ alkoxy, optionally substituted 3 to 6-membered cycloalkyl, or optionally substituted 3 to 6-membered heterocycloalkyl;
t is 0, 1, 2, or 3;
z is 0, 1, or 2;
X⁹ is —NR^{L6}, —C(O)—, or —S(O)₂—; and
each of R^{L1}, R^{L2}, R^{L3}, R^{L4}, R^{L4}, R^{L5}, and R^{L6} is, independently, hydrogen, halogen, hydroxyl, optionally substituted C₁-C₆ alkyl, optionally substituted C₂-C₁ alkenyl, optionally substituted C₂-C₆ alkynyl, or optionally substituted C₁-C₆ heteroalkyl; or any two of R^{L1}, R^{L2}, R^{L3}, R^{L4}, R^{L4}, R^{L5}, and R^{L6} together with the atoms to which they are attached and any intervening atoms to form an optionally substituted C₃-C₈ cycloalkyl or a 3- to 8-membered heterocyclyl.

E14. The compound of embodiment 13 having the structure of Formula Ia-1:

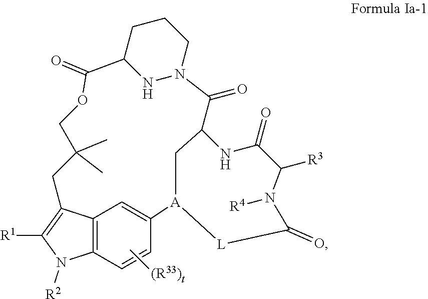

or a pharmaceutically acceptable salt thereof, wherein
A is optionally substituted 3- to 6-membered heterocycloalkylene, optionally substituted 3- to 6-membered cycloalkylene, optionally substituted 6-membered arylene, or optionally substituted 5- to 10-membered heteroarylene;
L is a linker;
R¹ is optionally substituted 5- to 10-membered heteroaryl;
R² is optionally substituted C₁-C₆ alkyl;
R³ is optionally substituted C₁-C₆ alkyl, optionally substituted C₁-C₃ heteroalkyl, or optionally substituted 3- to 6-membered cycloalkyl;
R⁴ is hydrogen or optionally substituted C₁-C₆ alkyl;
each R³³ is, independently, halogen, optionally substituted C₁-C₃ alkyl, optionally substituted C₁-C₃ alkoxy, optionally substituted 3 to 6-membered cycloalkyl, or optionally substituted 3 to 6-membered heterocycloalkyl; and
t is 0, 1, 2, or 3.

E15. The compound of embodiment E14 having the structure of Formula Ia-2:

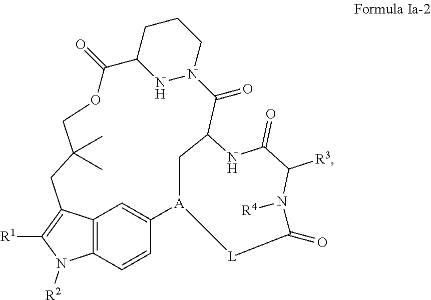

or a pharmaceutically acceptable salt thereof, wherein
A is optionally substituted 3- to 6-membered heterocycloalkylene, optionally substituted 3- to 6-membered cycloalkylene, optionally substituted 6-membered arylene, or optionally substituted 5- to 10-membered heteroarylene;
L is a linker;
R¹ is optionally substituted 5- to 10-membered heteroaryl;
R² is optionally substituted C₁-C₆ alkyl;
R³ is optionally substituted C₁-C₆ alkyl, optionally substituted C₁-C₃ heteroalkyl, or optionally substituted 3- to 6-membered cycloalkyl; and
R⁴ is hydrogen or optionally substituted C₁-C₆ alkyl.

E16. The compound of embodiment E13 wherein the compound has the structure of Formula IIa or Formula IIb:

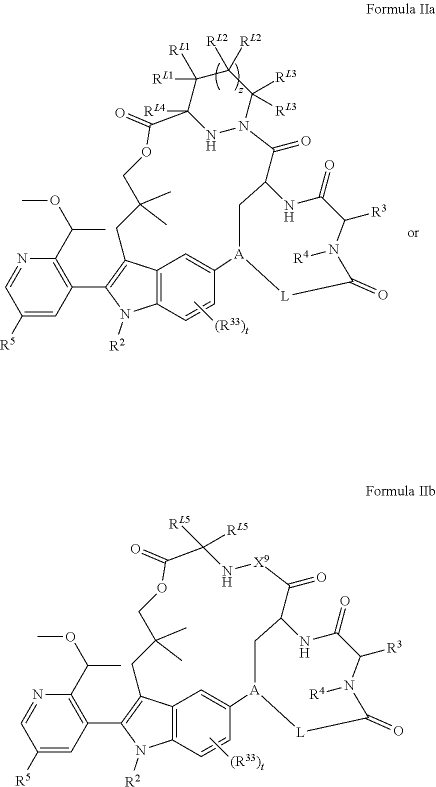

or a pharmaceutically acceptable salt thereof, wherein
R⁵ is hydrogen, optionally substituted 3- to 10-membered heterocycloalkyl, —OR^{5a}, or optionally substituted C₁-C₆ heteroalkyl; and
R^{5a} is optionally substituted C₁-C₆ alkyl or optionally substituted 5- to 10-membered heteroaryl.

E17. The compound of embodiment 16 wherein the compound has the structure of Formula IIa-1:

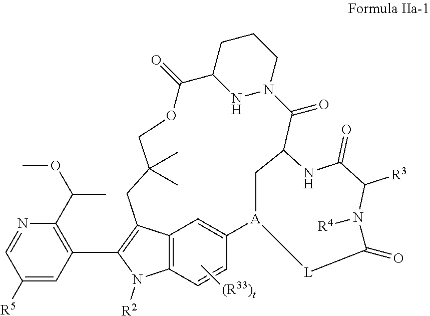

or a pharmaceutically acceptable salt thereof, wherein
R⁵ is hydrogen, optionally substituted 3- to 10-membered heterocycloalkyl, —OR^{5a}, or optionally substituted C₁-C₆ heteroalkyl; and
R^{5a} is optionally substituted C₁-C₆ alkyl or optionally substituted 5- to 10-membered heteroaryl;
each R³³ is, independently, halogen, optionally substituted C₁-C₃ alkyl, optionally substituted C₁-C₃ alkoxy, optionally substituted 3 to 6-membered cycloalkyl, or optionally substituted 3 to 6-membered heterocycloalkyl; and
t is 0, 1, 2, or 3.

E18. The compound of embodiment 17 wherein the compound has the structure of Formula IIa-2:

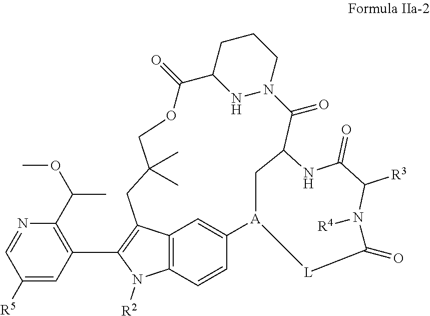

or a pharmaceutically acceptable salt thereof, wherein
R⁵ is hydrogen, optionally substituted 3- to 10-membered heterocycloalkyl, —OR^{5a}, or optionally substituted C₁-C₆ heteroalkyl; and
R^{5a} is optionally substituted C₁-C₆ alkyl or optionally substituted 5- to 10-membered heteroaryl.

E19. The compound of embodiment 16 wherein the compound has the structure of Formula IIIa or Formula IIIb:

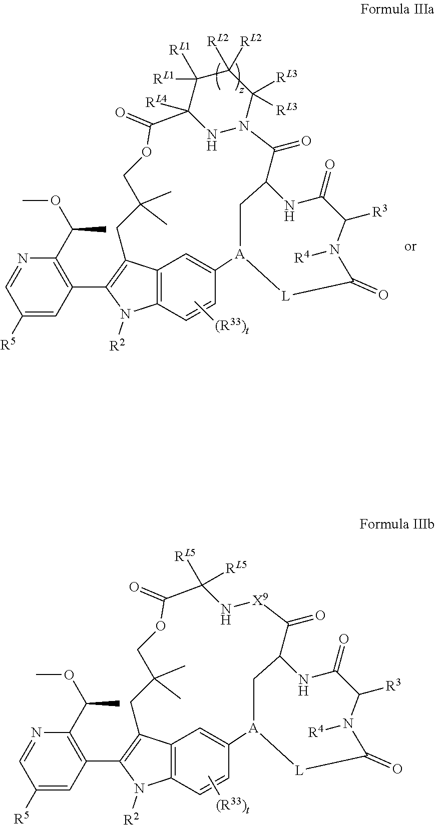

or a pharmaceutically acceptable salt thereof.

E20. The compound of embodiment 19 wherein the compound has the structure of Formula IIIa-1:

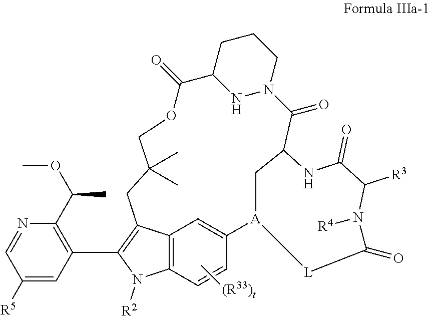

or a pharmaceutically acceptable salt thereof.

E21. The compound of embodiment 20 wherein the compound has the structure of Formula IIIa-2:

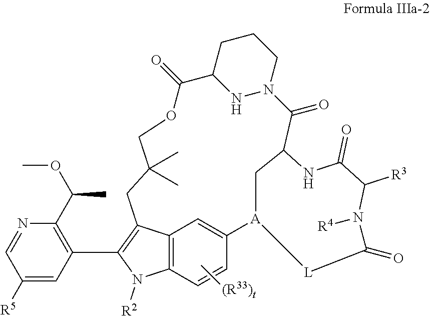

or a pharmaceutically acceptable salt thereof.

E22. The compound of any one of embodiments 13 to 21, or a pharmaceutically acceptable salt thereof, wherein
L has the structure of Formula IV:

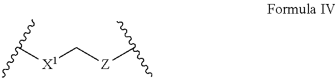

X¹ is O or CH₂ and is attached to ring A; and
Z is optionally substituted 3- to 6-membered heterocycloalkylene, optionally substituted C₁-C₆ alkylene, or optionally substituted C₁-C₆ heteroalkylene.

E23. The compound of embodiment 22 wherein the compound has the structure of Formula Va or Formula Vb:

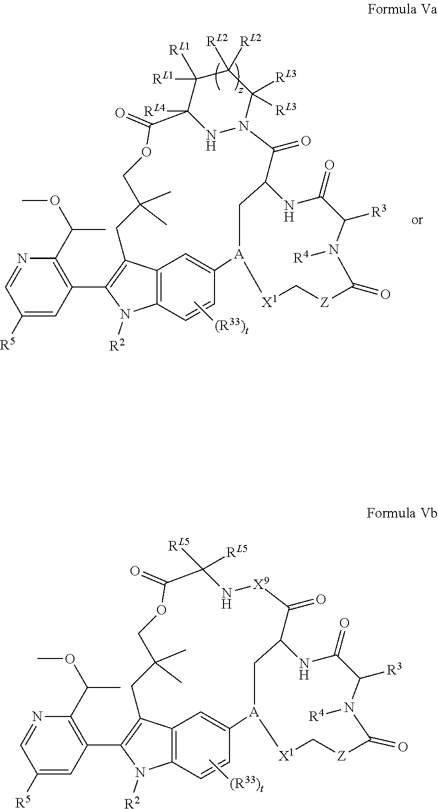

or a pharmaceutically acceptable salt thereof, wherein
X¹ is O or CH₂; and
Z is optionally substituted 3- to 6-membered heterocycloalkylene, optionally substituted C₁-C₆ alkylene, or optionally substituted C₁-C₆ heteroalkylene.

E24. The compound of embodiment 23 wherein the compound has the structure of Formula Va-1:

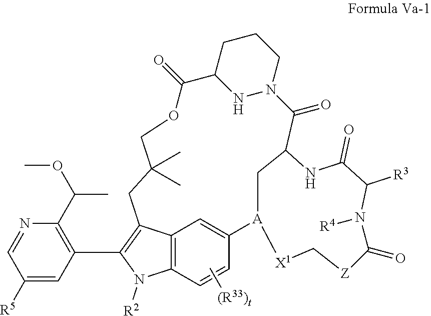

or a pharmaceutically acceptable salt thereof, wherein
X¹ is O or CH₂; and
Z is optionally substituted 3- to 6-membered heterocycloalkylene, optionally substituted C₁-C₆ alkylene, or optionally substituted C₁-C₆ heteroalkylene.

E25. The compound of embodiment 24 wherein the compound has the structure of Formula Va-2:

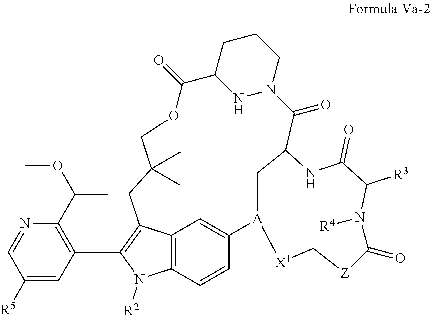

or a pharmaceutically acceptable salt thereof, wherein
X¹ is O or CH₂; and
Z is optionally substituted 3- to 6-membered heterocycloalkylene, optionally substituted C₁-C₆ alkylene, or optionally substituted C₁-C₆ heteroalkylene.

E26. The compound of any one of embodiments 22 to 25, or a pharmaceutically acceptable salt thereof, wherein X¹ is O.

E27. The compound of any one of embodiments 22 to 25, or a pharmaceutically acceptable salt thereof, wherein X¹ is CH₂.

E28. The compound of any one of embodiments 22 to 27, or a pharmaceutically acceptable salt thereof, wherein Z is optionally substituted 3- to 6-membered heterocycloalkylene.

E29. The compound of embodiment 28, or a pharmaceutically acceptable salt thereof, wherein Z is optionally substituted 5-membered heterocycloalkylene.

E30. The compound of embodiment 28, or a pharmaceutically acceptable salt thereof, wherein Z is optionally substituted pyrollidine-diyl.

E31. The compound of embodiment 28, or a pharmaceutically acceptable salt thereof, wherein Z is optionally substituted 6-membered heterocycloalkylene.

E32. The compound of embodiment 28, or a pharmaceutically acceptable salt thereof, wherein Z is optionally substituted piperidinyl.

E33. The compound of embodiment 28, or a pharmaceutically acceptable salt thereof, wherein Z is optionally substituted pyrrolidinyl.

E34. The compound of any one of embodiments 13 to 21, or a pharmaceutically acceptable salt thereof, wherein
L has the structure of Formula VI:

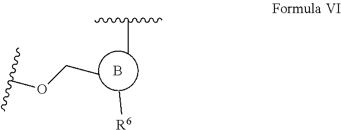

B is an optionally substituted 3- to 6-membered heterocycloalkylene;
R⁶ is hydrogen, optionally substituted C₁-C₆ alkyl, optionally substituted C₁-C₆ heteroalkyl, optionally substituted 3- to 6-membered heterocyclyl, optionally substituted 3- to 6-membered cycloalkyl, optionally substituted 5- to 10-membered heteroaryl, optionally substituted C₆-C₁₀ aryl,

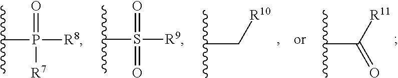

R⁷ and R⁸ are each, independently, H or optionally substituted C₁-C₆ alkyl;
R⁹ is optionally substituted C₁-C₆ alkyl, optionally substituted C₂-C₆ alkenyl, optionally substituted 3- to 6-membered cycloalkyl, or optionally substituted 3- to 6-membered heterocyclyl;
R¹⁰ is optionally substituted C₁-C₆ alkyl, optionally substituted C₂-C₆ alkenyl, optionally substituted C₁-C₆ heteroalkyl, optionally substituted C₃-C₁₀ cycloalkyl, optionally substituted 3- to 6-membered heterocyclyl, optionally substituted 5- to 10-membered heteroaryl, or optionally substituted C₆-C₁₀ aryl; and
R¹¹ is hydrogen, optionally substituted C₁-C₆ alkyl, optionally substituted C₁-C₆ heteroalkyl, optionally substituted C₂-C₆ alkenyl, optionally substituted C₂-C₆ heteroalkenyl, optionally substituted C₂-C₆ alkynyl, optionally substituted C₂-C₆ heteroalkynyl, optionally substituted C₃-C₁₀ cycloalkyl, optionally substituted 3- to 10-membered heterocyclyl, optionally substituted C₃-C₁₀ cycloalkenyl, optionally substituted 3- to 10-membered heterocycloalkenyl, optionally substituted C₆-C₁₀ aryl, or optionally substituted 5- to 10-membered heteroaryl.

E35. The compound of embodiment 34, or a pharmaceutically acceptable salt thereof, wherein L has the structure of Formula VIa:

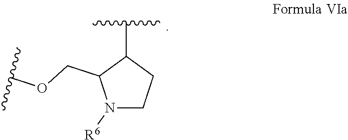

E36. The compound of embodiment 35 wherein the compound has the structure of Formula VIIa or Formula VIIb:

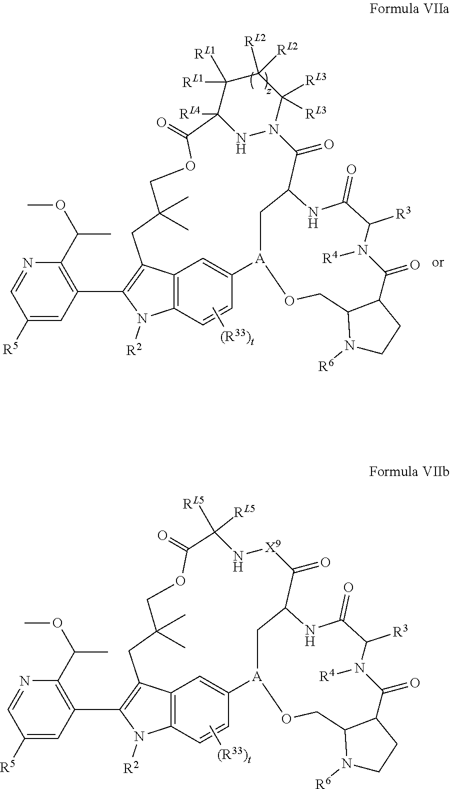

or a pharmaceutically acceptable salt thereof, wherein
R⁶ is hydrogen, optionally substituted C₁-C₆ alkyl, optionally substituted C₁-C₆ heteroalkyl, optionally substituted 3- to 6-membered heterocyclyl, optionally substituted 3- to 6-membered cycloalkyl, optionally substituted 5- to 10-membered heteroaryl, optionally substituted C₆-C₁₀ aryl,

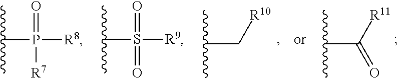

R⁷ and R⁸ are each, independently, H or optionally substituted C₁-C₆ alkyl;
R⁹ is optionally substituted C₁-C₆ alkyl, optionally substituted C₂-C₆ alkenyl, optionally substituted 3- to 6-membered cycloalkyl, or optionally substituted 3- to 6-membered heterocyclyl;
R¹⁰ is optionally substituted C₁-C₆ alkyl, optionally substituted C₂-C₆ alkenyl, optionally substituted C₁-C₆ heteroalkyl, optionally substituted C₃-C₁₀ cycloalkyl, optionally substituted 3- to 6-membered heterocyclyl, optionally substituted 5- to 10-membered heteroaryl, or optionally substituted C₆-C₁₀ aryl; and
R¹¹ is hydrogen, optionally substituted C₁-C₆ alkyl, optionally substituted C₁-C₆ heteroalkyl, optionally substituted C₂-C₆ alkenyl, optionally substituted C₂-C₆ heteroalkenyl, optionally substituted C₂-C₆ alkynyl, optionally substituted C₂-C₆ heteroalkynyl, optionally substituted C₃-C₁₀ cycloalkyl, optionally substituted 3- to 10-membered heterocyclyl, optionally substituted C₃-C₁₀ cycloalkenyl, optionally substituted 3- to 10-membered heterocycloalkenyl, optionally substituted C₆-C₁₀ aryl, or optionally substituted 5- to 10-membered heteroaryl.

E37. The compound of embodiment 36 wherein the compound has the structure of Formula VIIa-1:

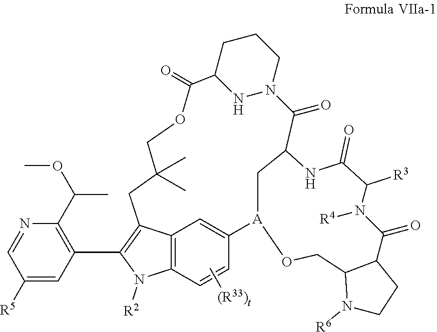

or a pharmaceutically acceptable salt thereof, wherein
R⁶ is hydrogen, optionally substituted C₁-C₆ alkyl, optionally substituted C₁-C₆ heteroalkyl, optionally substituted 3- to 6-membered heterocyclyl, optionally substituted 3- to 6-membered cycloalkyl, optionally substituted 5- to 10-membered heteroaryl, optionally substituted C₆-C₁₀ aryl,

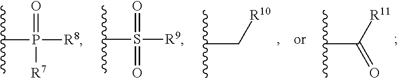

R⁷ and R⁸ are each, independently, H or optionally substituted C₁-C₆ alkyl;
R⁹ is optionally substituted C₁-C₆ alkyl, optionally substituted C₂-C₆ alkenyl, optionally substituted 3- to 6-membered cycloalkyl, or optionally substituted 3- to 6-membered heterocyclyl;
R¹⁰ is optionally substituted C₁-C₆ alkyl, optionally substituted C₂-C₆ alkenyl, optionally substituted C₁-C₆ heteroalkyl, optionally substituted C₃-C₁₀ cycloalkyl, optionally substituted 3- to 6-membered heterocyclyl, optionally substituted 5- to 10-membered heteroaryl, or optionally substituted C₆-C₁₀ aryl; and
R¹¹ is hydrogen, optionally substituted C₁-C₆ alkyl, optionally substituted C₁-C₆ heteroalkyl, optionally substituted C₂-C₆ alkenyl, optionally substituted C₂-C₆ heteroalkenyl, optionally substituted C₂-C₆ alkynyl, optionally substituted C₂-C₆ heteroalkynyl, optionally substituted C₃-C₁₀ cycloalkyl, optionally substituted 3- to 10-membered heterocyclyl, optionally substituted C₃-C₁₀ cycloalkenyl, optionally substituted 3- to 10-membered heterocycloalkenyl, optionally substituted C₆-C₁₀ aryl, or optionally substituted 5- to 10-membered heteroaryl.

E38. The compound of embodiment 37 wherein the compound has the structure of Formula VIIa-2:

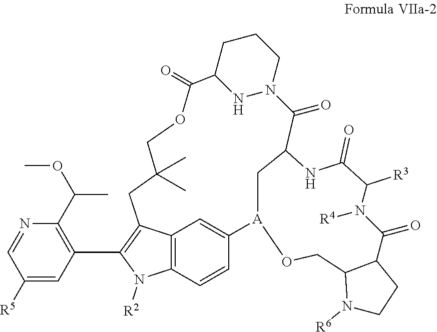

or a pharmaceutically acceptable salt thereof, wherein
R⁶ is hydrogen, optionally substituted C₁-C₆ alkyl, optionally substituted C₁-C₆ heteroalkyl, optionally substituted 3- to 6-membered heterocyclyl, optionally substituted 3- to 6-membered cycloalkyl, optionally substituted 5- to 10-membered heteroaryl, optionally substituted C₆-C₁₀ aryl,

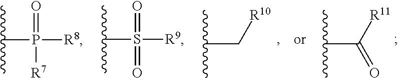

R⁷ and R⁸ are each, independently, H or optionally substituted C₁-C₆ alkyl;
R⁹ is optionally substituted C₁-C₆ alkyl, optionally substituted C₂-C₆ alkenyl, optionally substituted 3- to 6-membered cycloalkyl, or optionally substituted 3- to 6-membered heterocyclyl;
R¹⁰ is optionally substituted C₁-C₆ alkyl, optionally substituted C₂-C₆ alkenyl, optionally substituted C₁-C₆ heteroalkyl, optionally substituted C₃-C₁₀ cycloalkyl, optionally substituted 3- to 6-membered heterocyclyl, optionally substituted 5- to 10-membered heteroaryl, or optionally substituted C₆-C₁₀ aryl; and
R¹¹ is hydrogen, optionally substituted C₁-C₆ alkyl, optionally substituted C₁-C₆ heteroalkyl, optionally substituted C₂-C₆ alkenyl, optionally substituted C₂-C₆ heteroalkenyl, optionally substituted C₂-C₆ alkynyl, optionally substituted C₂-C₆ heteroalkynyl, optionally substituted C₃-C₁₀ cycloalkyl, optionally substituted 3- to 10-membered heterocyclyl, optionally substituted C₃-C₁₀ cycloalkenyl, optionally substituted 3- to 10-membered heterocycloalkenyl, optionally substituted C₆-C₁₀ aryl, or optionally substituted 5- to 10-membered heteroaryl.

E39. The compound of any one of embodiments 10 to 12, and 34 to 38, or a pharmaceutically acceptable salt thereof, wherein R⁶ is

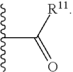

E40. The compound of any one of embodiments 10 to 12 and 34 to 39, or a pharmaceutically acceptable salt thereof, wherein R¹¹ is optionally substituted C₁-C₆ alkyl.

E41. The compound of any one of embodiments 10 to 12 and 34 to 39, or a pharmaceutically acceptable salt thereof, wherein R¹¹ is optionally substituted C₂-C₆ alkenyl.

E42. The compound of any one of embodiments 10 to 12 and 34 to 39, or a pharmaceutically acceptable salt thereof, wherein R¹¹ is optionally substituted C₂-C₆ alkynyl.

E43. The compound of any one of embodiments 10 to 12 and 34 to 39, or a pharmaceutically acceptable salt thereof, wherein R¹¹ is optionally substituted C₁-C₆ heteroalkyl.

E44. The compound of any one of embodiments 10 to 12 and 34 to 39, or a pharmaceutically acceptable salt thereof, wherein R¹¹ is optionally substituted C₂-C₆ heteroalkenyl.

E45. The compound of any one of embodiments 10 to 12 and 34 to 39, or a pharmaceutically acceptable salt thereof, wherein R¹¹ is optionally substituted C₂-C₆ heteroalkynyl.

E46. The compound of any one of embodiments 10 to 12 and 34 to 39, or a pharmaceutically acceptable salt thereof, wherein R¹¹ is optionally substituted C₃-C₁₀ cycloalkenyl.

E47. The compound of any one of embodiments 10 to 12 and 34 to 39, or a pharmaceutically acceptable salt thereof, wherein R¹¹ is hydrogen.

E48. The compound of any one of embodiments 10 to 12 and 34 to 39, or a pharmaceutically acceptable salt thereof, wherein R¹¹ is optionally substituted C₃-C₁₀ cycloalkyl.

E49. The compound of any one of embodiments 10 to 12 and 34 to 39, or a pharmaceutically acceptable salt thereof, wherein R¹¹ is optionally substituted 3- to 10-membered heterocyclyl.

E50. The compound of any one of embodiments 10 to 12 and 34 to 38, or a pharmaceutically acceptable salt thereof, wherein R⁶ is

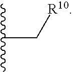

E51. The compound of embodiment 10 to 12, 34, 36, and 50 and 55, or a pharmaceutically acceptable salt thereof, wherein R¹⁰ is optionally substituted 5- to 10-membered heteroaryl.

E52. The compound of embodiment 10 to 12, 34, 36, and 50, or a pharmaceutically acceptable salt thereof, wherein R¹⁰ is optionally substituted 3- to 10-membered heterocyclyl.

E53. The compound of any one of embodiments 10 to 12 and 34 to 38, or a pharmaceutically acceptable salt thereof, wherein R⁶ is optionally substituted 3- to 6-membered heterocyclyl.

E54. The compound of any one of embodiments 1 to 53, or a pharmaceutically acceptable salt thereof, wherein A is optionally substituted 3- to 6-membered heterocycloalkylene, optionally substituted 6-membered arylene, or optionally substituted 5- to 10-membered heteroarylene.

E55. The compound of any one of embodiments 1 to 53, or a pharmaceutically acceptable salt thereof, wherein A is optionally substituted 6-membered arylene.

E56. The compound of any one of embodiments 1 to 59, or a pharmaceutically acceptable salt thereof, wherein A is:

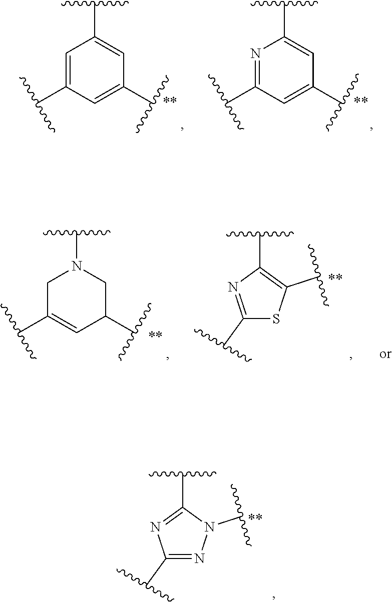 [SMILES: *c1cc(*)cc(*)c1 ; *c1cc(*)nc(*)c1 ; *C1=CC(*)CN(*)C1 ; *c1nc(*)c(*)s1 ; *c1nc(*)n(*)n1]

wherein

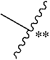

represents the portion of the molecule bound to the linker.

E56. The compound of embodiment 56, or a pharmaceutically acceptable salt thereof, wherein A is

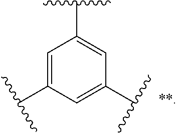 [SMILES: *c1cc(*)cc(*)c1]

E57. The compound of embodiment 56, or a pharmaceutically acceptable salt thereof, wherein A is

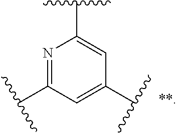 [SMILES: *c1cc(*)nc(*)c1]

E58. The compound of any one of embodiments 1 to 57, or a pharmaceutically acceptable salt thereof, wherein R² is C₁-C₃ alkyl or C₁-C₃ haloalkyl.

E59. The compound of embodiment 58, or a pharmaceutically acceptable salt thereof, wherein R² is

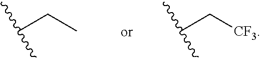

E60. The compound of embodiment 59, or a pharmaceutically acceptable salt thereof, wherein R² is

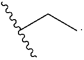 [SMILES: *CC]

E61. The compound of any one of embodiments 1 to 60, or a pharmaceutically acceptable salt thereof, wherein R⁴ is optionally substituted C₁-C₆ alkyl.

E62. The compound of embodiment 61, or a pharmaceutically acceptable salt thereof, wherein R⁴ is methyl.

E63. The compound of embodiment 61, or a pharmaceutically acceptable salt thereof, wherein R⁴ is ethyl.

E64. The compound of embodiment 61, or a pharmaceutically acceptable salt thereof, wherein R⁴ is isopropyl.

E65. The compound of any one of embodiments 1 to 64, or a pharmaceutically acceptable salt thereof, wherein R³ is optionally substituted C₁-C₆ alkyl.

E66. The compound of any one of embodiments 1 to 64, or a pharmaceutically acceptable salt thereof, wherein R³ is optionally substituted 3- to 6-membered cycloalkyl.

E67. The compound of any one of embodiments 1 to 64, or a pharmaceutically acceptable salt thereof, wherein R³ is

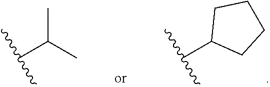

E68. The compound of any one of embodiments 1 to 64, or a pharmaceutically acceptable salt thereof, wherein R³ is methyl.

E69. The compound of any one of embodiments 2 to 12 and 16 to 68, or a pharmaceutically acceptable salt thereof, wherein R⁵ is hydrogen.

E70. The compound of any one of embodiments 2 to 12 and 16 to 68, or a pharmaceutically acceptable salt thereof, wherein R⁵ is optionally substituted 3- to 10-membered heterocycloalkyl.

E71. The compound of embodiment 70, or a pharmaceutically acceptable salt thereof, wherein R⁵

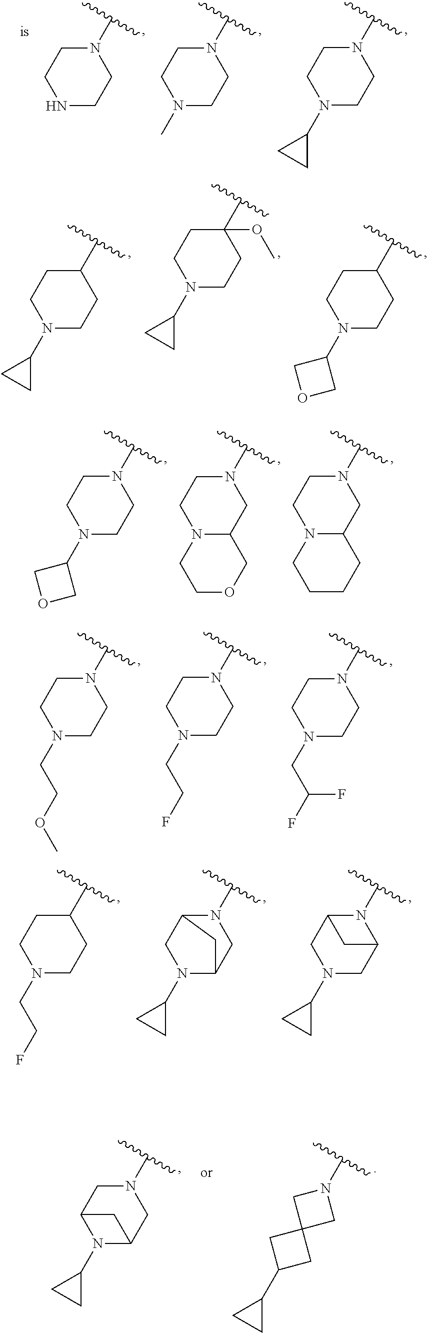

E72. The compound of embodiment 70, or a pharmaceutically acceptable salt thereof, wherein R⁵ is

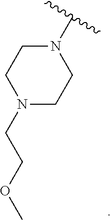 [SMILES: *N1CCN(CCOC)CC1]

E73. The compound of embodiment 70, or a pharmaceutically acceptable salt thereof, wherein R⁵ is

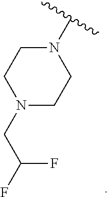 [SMILES: *N1CCN(CC(F)F)CC1]

E74. The compound of any one of embodiments 2 to 12 and 16 to 68, or a pharmaceutically acceptable salt thereof, wherein R⁵ is —OR^{5a}.

E75. The compound of any one of embodiments 2 to 12 and 16 to 68, or a pharmaceutically acceptable salt thereof, wherein R⁵ is

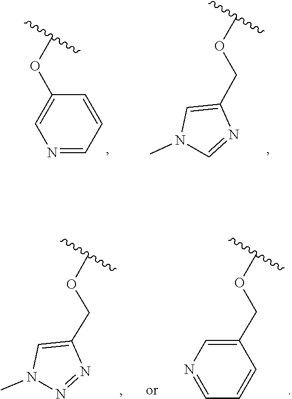

E76. A compound A1-A450, or a pharmaceutically acceptable salt thereof, of Table 1.

E77. A compound B1-B35, or a pharmaceutically acceptable salt thereof, of Table 2.

E78. A pharmaceutical composition comprising a compound, or a pharmaceutically acceptable salt thereof, of any one of embodiments 1-77 and a pharmaceutically acceptable excipient.

E79. A method of treating cancer in a subject in need thereof, the method comprising administering to the subject a therapeutically effective amount of a compound, or a pharmaceutically acceptable salt thereof, of any one of embodiments 1-77 or a pharmaceutical composition of embodiment 56.

E80. The method of embodiment 79, wherein the cancer is pancreatic cancer, colorectal cancer, non-small cell lung cancer, multiple myeloma, or acute myeloid leukemia.

E81. The method of embodiment 79 or 80, wherein the cancer comprises a Ras mutation.

E82. The method of embodiment 81, wherein the Ras mutation is K-Ras Q61H, H-Ras Q61H, or N-Ras Q61H.

E83. The method of embodiment 82, wherein the Ras mutation is K-Ras Q61H.

E84. A method of treating a Ras protein-related disorder in a subject in need thereof, the method comprising administering to the subject a therapeutically effective amount of a compound of any one of embodiments 1-77, or a pharmaceutically acceptable salt thereof, or a pharmaceutical composition of embodiment 78.

E85. A method of inhibiting a Ras protein in a cell, the method comprising contacting the cell with an effective amount of a compound of any one of embodiments 1 to 77, or a pharmaceutically acceptable salt thereof, or a pharmaceutical composition of embodiment 78.

E86. The method of embodiment 84 or 85, wherein the Ras protein K-Ras Q61H, H-Ras Q61H, or N-Ras Q61H.

E87. The method of embodiment 86, wherein the Ras protein is K-Ras Q61H.

E88. The method of any one of embodiments 85 to 87, wherein the cell is a cancer cell.

E89. The method of embodiment 88, wherein the cancer cell is a pancreatic cancer cell, a colorectal cancer cell, a non-small cell lung cancer cell, a multiple myeloma cancer cell, or an acute myeloid leukemia cancer cell.

E90. The method of any one of embodiments, wherein the method further comprises administering an additional anti-cancer therapy.

E91. The method of embodiment 90, wherein the additional anti-cancer therapy is an EGFR inhibitor, a second Ras inhibitor, a SHP2 inhibitor, a SOS1 inhibitor, a Raf inhibitor, a MEK inhibitor, an ERK inhibitor, a PI3K inhibitor, a PTEN inhibitor, an AKT inhibitor, an mTORC1 inhibitor, a BRAF inhibitor, a PD-L1 inhibitor, a PD-1 inhibitor, a CDK4/6 inhibitor, a HER2 inhibitor, or a combination thereof.

E92. The method of embodiment 90 or 91, wherein the additional anti-cancer therapy is a SHP2 inhibitor.

1. A compound having the structure of Formula Ia or Formula Ib: 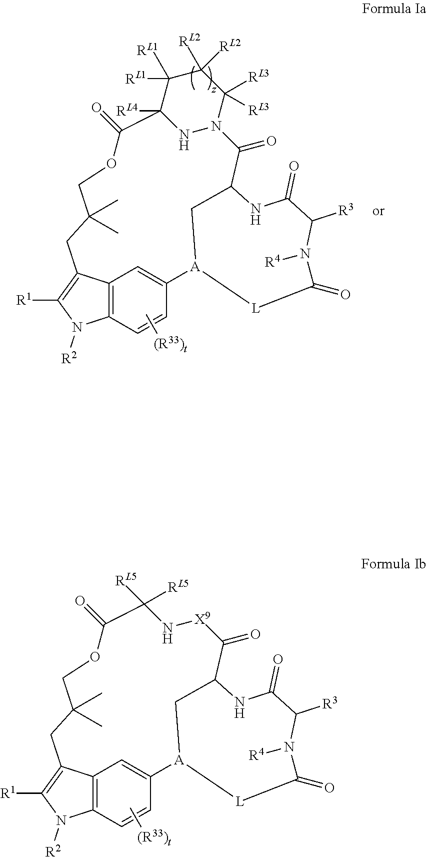
or a pharmaceutically acceptable salt thereof, wherein
A is optionally substituted 3- to 6-membered heterocycloalkylene, optionally substituted 3- to 6-membered cycloalkylene, optionally substituted 6-membered arylene, or optionally substituted 5- to 10-membered heteroarylene;
L is a linker;
R¹ is optionally substituted 5- to 10-membered heteroaryl;
R² is optionally substituted C₁-C₆ alkyl;
R³ is optionally substituted C₁-C₆ alkyl, optionally substituted C₁-C₃ heteroalkyl, or optionally substituted 3- to 6-membered cycloalkyl;
R⁴ is hydrogen or optionally substituted C₁-C₆ alkyl;
each R³³ is, independently, halogen, optionally substituted C₁-C₃ alkyl, optionally substituted C₁-C₃ alkoxy, optionally substituted 3 to 6-membered cycloalkyl, or optionally substituted 3 to 6-membered heterocycloalkyl;
t is 0, 1, 2, or 3;
z is 0, 1, or 2;
X⁹ is —NR^{L6}—, —C(O)—, or —S(O)₂—; and
each of R^{L1}, R^{L2}, R^{L3}, R^{L4}, R^{L4}, R^{L5}, and R^{L6} is, independently, hydrogen, halogen, hydroxyl, optionally substituted C₁-C₆ alkyl, optionally substituted C₂-C₆ alkenyl, optionally substituted C₂-C₆ alkynyl, or optionally substituted C₁-C₆ heteroalkyl; or any two of R^{L1}, R^{L2}, R^{L3}, R^{L4}, R^{L4}, R^{L5}, and R^{L6} together with the atoms to which they are attached and any intervening atoms to form an optionally substituted C₃-C₈ cycloalkyl or a 3- to 8-membered heterocyclyl.

2. The compound of claim 1 having the structure of Formula Ia-1: 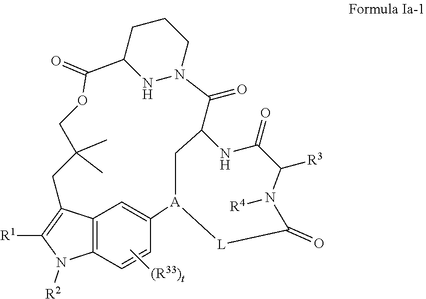
or a pharmaceutically acceptable salt thereof, wherein
A is optionally substituted 3- to 6-membered heterocycloalkylene, optionally substituted 3- to 6-membered cycloalkylene, optionally substituted 6-membered arylene, or optionally substituted 5- to 10-membered heteroarylene;
L is a linker;
R¹ is optionally substituted 5- to 10-membered heteroaryl;
R² is optionally substituted C₁-C₆ alkyl;
R³ is optionally substituted C₁-C₆ alkyl, optionally substituted C₁-C₃ heteroalkyl, or optionally substituted 3- to 6-membered cycloalkyl;
R⁴ is hydrogen or optionally substituted C₁-C₆ alkyl;
each R³³ is, independently, halogen, optionally substituted C₁-C₃ alkyl, optionally substituted C₁-C₃ alkoxy, optionally substituted 3 to 6-membered cycloalkyl, or optionally substituted 3 to 6-membered heterocycloalkyl; and
t is 0, 1, 2, or 3.

3. The compound of claim 2 having the structure of Formula Ia-2: 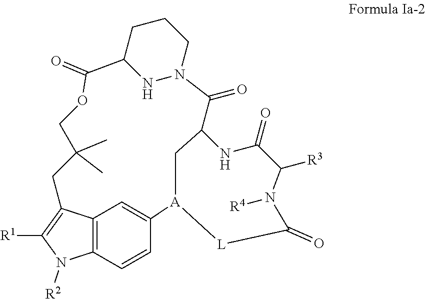
or a pharmaceutically acceptable salt thereof, wherein
A is optionally substituted 3- to 6-membered heterocycloalkylene, optionally substituted 3- to 6-membered cycloalkylene, optionally substituted 6-membered arylene, or optionally substituted 5- to 10-membered heteroarylene;
L is a linker;
R¹ is optionally substituted 5- to 10-membered heteroaryl;
R² is optionally substituted C₁-C₆ alkyl;
R³ is optionally substituted C₁-C₆ alkyl, optionally substituted C₁-C₃ heteroalkyl, or optionally substituted 3- to 6-membered cycloalkyl; and
R⁴ is hydrogen or optionally substituted C₁-C₆ alkyl.

4. The compound of claim 1 wherein the compound has the structure of Formula IIa or Formula IIb: 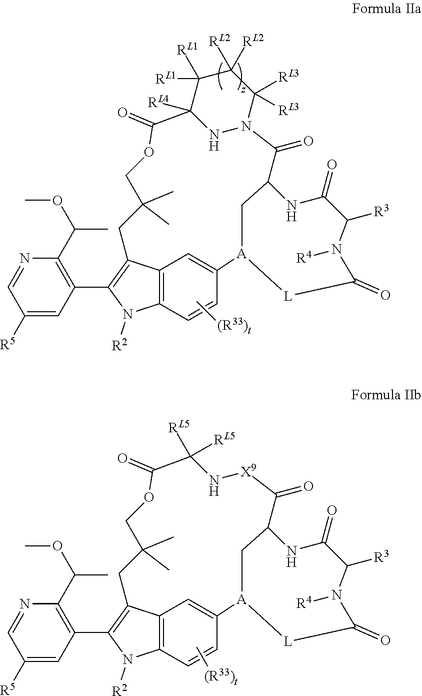
or a pharmaceutically acceptable salt thereof, wherein
R⁵ is hydrogen, optionally substituted 3- to 10-membered heterocycloalkyl, —OR^{5a}, or optionally substituted C₁-C₆ heteroalkyl; and
R^{5a} is optionally substituted C₁-C₆ alkyl or optionally substituted 5- to 10-membered heteroaryl.

5. The compound of claim 4 wherein the compound has the structure of Formula IIa-1: 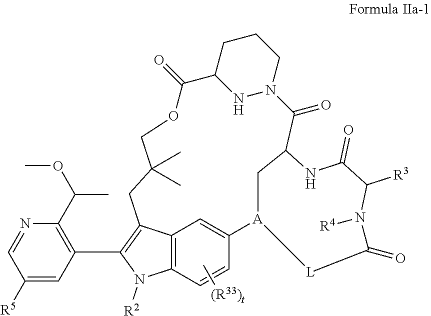
or a pharmaceutically acceptable salt thereof, wherein
R⁵ is hydrogen, optionally substituted 3- to 10-membered heterocycloalkyl, —OR^{5a}, or optionally substituted C₁-C₆ heteroalkyl; and
R^{5a} is optionally substituted C₁-C₆ alkyl or optionally substituted 5- to 10-membered heteroaryl;
each R³³ is, independently, halogen, optionally substituted C₁-C₃ alkyl, optionally substituted C₁-C₃ alkoxy, optionally substituted 3 to 6-membered cycloalkyl, or optionally substituted 3 to 6-membered heterocycloalkyl; and
t is 0, 1, 2, or 3.

6. The compound of claim 5 wherein the compound has the structure of Formula IIa-2: 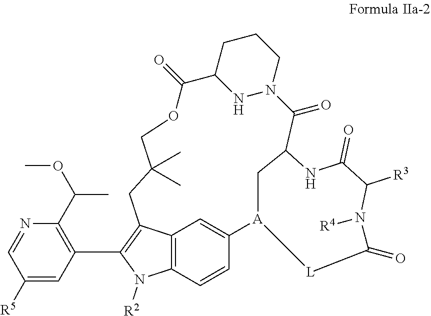
or a pharmaceutically acceptable salt thereof, wherein
R⁵ is hydrogen, optionally substituted 3- to 10-membered heterocycloalkyl, —OR^{5a}, or optionally substituted C₁-C₆ heteroalkyl; and
R^{5a} is optionally substituted C₁-C₆ alkyl or optionally substituted 5- to 10-membered heteroaryl.

7. The compound of claim 4 wherein the compound has the structure of Formula IIIa or Formula IIIb: 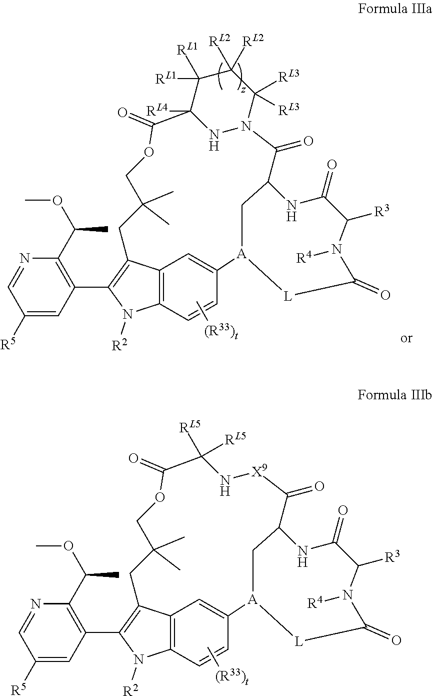
or a pharmaceutically acceptable salt thereof.

8. The compound of claim 7 wherein the compound has the structure of Formula IIIa-1: 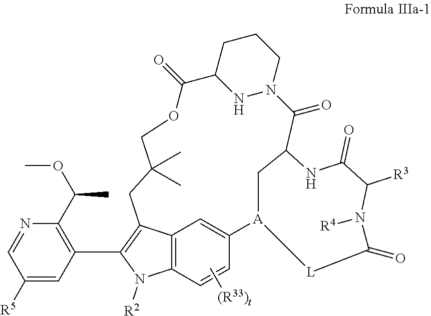
or a pharmaceutically acceptable salt thereof.

9. The compound of claim 8 wherein the compound has the structure of Formula IIIa-2: 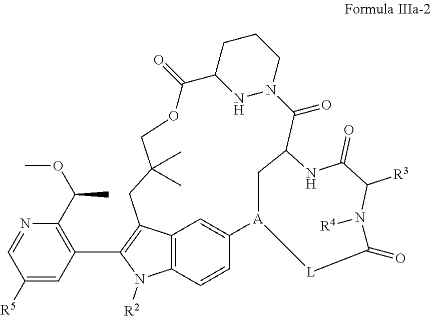
or a pharmaceutically acceptable salt thereof.

10. The compound of any one of claims 1 to 9, or a pharmaceutically acceptable salt thereof, wherein
L has the structure of Formula IV: 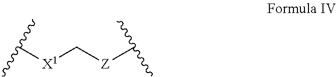
X¹ is O or CH₂ and is attached to ring A; and
Z is optionally substituted 3- to 6-membered heterocycloalkylene, optionally substituted C₁-C₆ alkylene, or optionally substituted C₁-C₆ heteroalkylene.

11. The compound of claim 10 wherein the compound has the structure of Formula Va or Formula Vb: 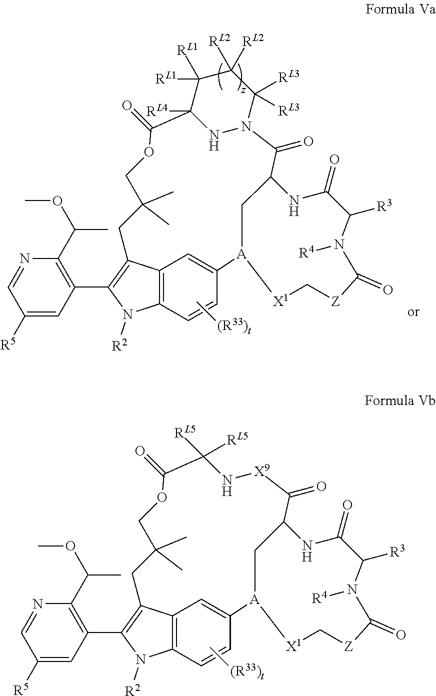
or a pharmaceutically acceptable salt thereof, wherein
X¹ is O or CH₂; and
Z is optionally substituted 3- to 6-membered heterocycloalkylene, optionally substituted C₁-C₆ alkylene, or optionally substituted C₁-C₆ heteroalkylene.

12. The compound of claim 11 wherein the compound has the structure of Formula Va-1: 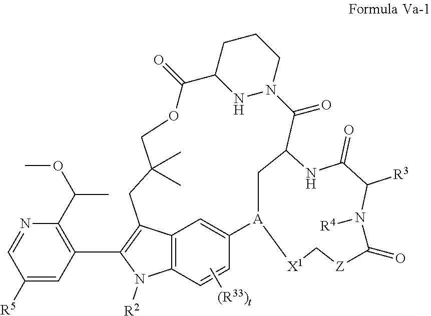
or a pharmaceutically acceptable salt thereof, wherein
X¹ is O or CH₂; and
Z is optionally substituted 3- to 6-membered heterocycloalkylene, optionally substituted C₁-C₆ alkylene, or optionally substituted C₁-C₆ heteroalkylene.

13. The compound of claim 12 wherein the compound has the structure of Formula Va-2: 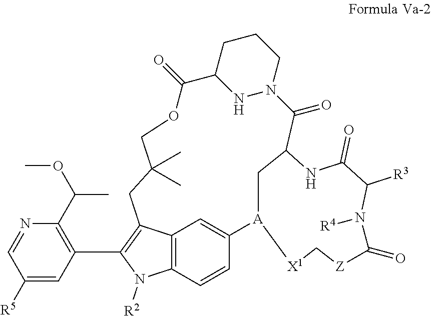
or a pharmaceutically acceptable salt thereof, wherein
X¹ is 0 or CH₂; and
Z is optionally substituted 3- to 6-membered heterocycloalkylene, optionally substituted C₁-C₆ alkylene, or optionally substituted C₁-C₆ heteroalkylene.

14. The compound of any one of claims 1 to 9, or a pharmaceutically acceptable salt thereof, wherein
L has the structure of Formula VI: 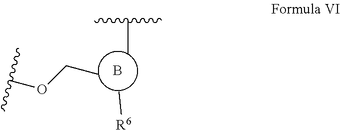
B is an optionally substituted 3- to 6-membered heterocycloalkylene;
R⁶ is hydrogen, optionally substituted C₁-C₆ alkyl, optionally substituted C₁-C₆ heteroalkyl, optionally substituted 3- to 6-membered heterocyclyl, optionally substituted 3- to 6-membered cycloalkyl, optionally substituted 5- to 10-membered heteroaryl, optionally substituted C₆-C₁₀ aryl 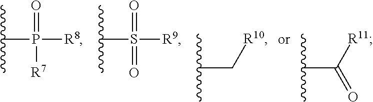
R⁷ and R⁸ are each, independently, H or optionally substituted C₁-C₆ alkyl;
R⁹ is optionally substituted C₁-C₆ alkyl, optionally substituted C₂-C₆ alkenyl, optionally substituted 3- to 6-membered cycloalkyl, or optionally substituted 3- to 6-membered heterocyclyl;
R¹⁰ is optionally substituted C₁-C₆ alkyl, optionally substituted C₂-C₆ alkenyl, optionally substituted C₁-C₆ heteroalkyl, optionally substituted C₃-C₁₀ cycloalkyl, optionally substituted 3- to 6-membered heterocyclyl, optionally substituted 5- to 10-membered heteroaryl, or optionally substituted C₆-C₁₀ aryl; and
R¹¹ is hydrogen, optionally substituted C₁-C₆ alkyl, optionally substituted C₁-C₆ heteroalkyl, optionally substituted C₂-C₆ alkenyl, optionally substituted C₂-C₆ heteroalkenyl, optionally substituted C₂-C₆ alkynyl, optionally substituted C₂-C₆ heteroalkynyl, optionally substituted C₃-C₁₀ cycloalkyl, optionally substituted 3- to 10-membered heterocyclyl, optionally substituted C₃-C₁₀ cycloalkenyl, optionally substituted 3- to 10-membered heterocycloalkenyl, optionally substituted C₆-C₁₀ aryl, or optionally substituted 5- to 10-membered heteroaryl.

15. The compound of claim 14, or a pharmaceutically acceptable salt thereof, wherein L has the structure of Formula VIa:

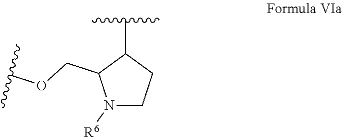

16. The compound of claim 15, wherein the compound has the structure of Formula VIIa or Formula VIIb: 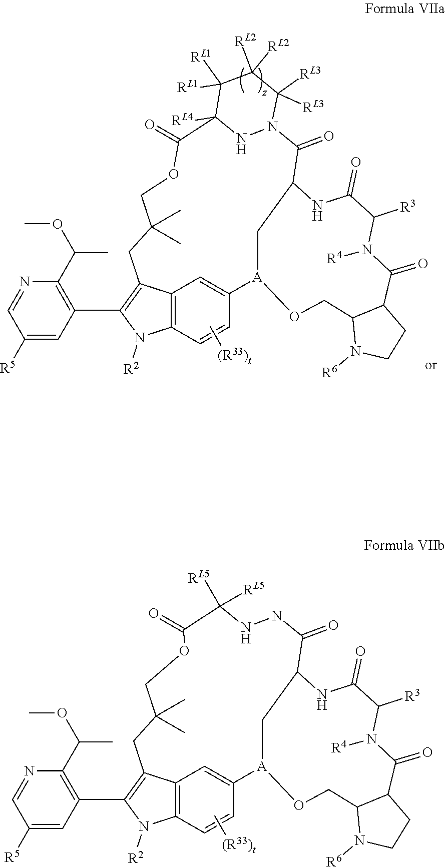
or a pharmaceutically acceptable salt thereof, wherein
R⁶ is hydrogen, optionally substituted C₁-C₆ alkyl, optionally substituted C₁-C₆ heteroalkyl, optionally substituted 3- to 6-membered heterocyclyl, optionally substituted 3- to 6-membered cycloalkyl, optionally substituted 5- to 10-membered heteroaryl, optionally substituted C₆-C₁₀ aryl, 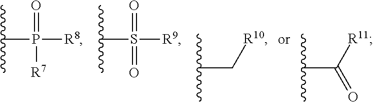
R⁷ and R⁸ are each, independently, H or optionally substituted C₁-C₆ alkyl;
R⁹ is optionally substituted C₁-C₆ alkyl, optionally substituted C₂-C₆ alkenyl, optionally substituted 3- to 6-membered cycloalkyl, or optionally substituted 3- to 6-membered heterocyclyl;
R¹⁰ is optionally substituted C₁-C₆ alkyl, optionally substituted C₂-C₆ alkenyl, optionally substituted C₁-C₆ heteroalkyl, optionally substituted C₃-C₁₀ cycloalkyl, optionally substituted 3- to 6-membered heterocyclyl, optionally substituted 5- to 10-membered heteroaryl, or optionally substituted C₆-C₁₀ aryl; and
R¹¹ is hydrogen, optionally substituted C₁-C₆ alkyl, optionally substituted C₁-C₆ heteroalkyl, optionally substituted C₂-C₆ alkenyl, optionally substituted C₂-C₆ heteroalkenyl, optionally substituted C₂-C₆ alkynyl, optionally substituted C₂-C₆ heteroalkynyl, optionally substituted C₃-C₁₀ cycloalkyl, optionally substituted 3- to 10-membered heterocyclyl, optionally substituted C₃-C₁₀ cycloalkenyl, optionally substituted 3- to 10-membered heterocycloalkenyl, optionally substituted C₆-C₁₀ aryl, or optionally substituted 5- to 10-membered heteroaryl.

17. The compound of claim 16, wherein the compound has the structure of Formula VIIa-1: 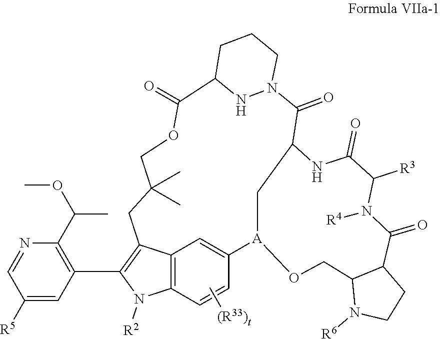
or a pharmaceutically acceptable salt thereof, wherein
R⁶ is hydrogen, optionally substituted C₁-C₆ alkyl, optionally substituted C₁-C₆ heteroalkyl, optionally substituted 3- to 6-membered heterocyclyl, optionally substituted 3- to 6-membered cycloalkyl, optionally substituted 5- to 10-membered heteroaryl, optionally substituted C₆-C₁₀ aryl, 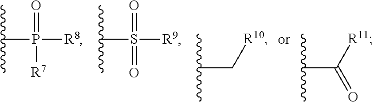
R⁷ and R⁸ are each, independently, H or optionally substituted C₁-C₆ alkyl;
R⁹ is optionally substituted C₁-C₆ alkyl, optionally substituted C₂-C₆ alkenyl, optionally substituted 3- to 6-membered cycloalkyl, or optionally substituted 3- to 6-membered heterocyclyl;
R¹⁰ is optionally substituted C₁-C₆ alkyl, optionally substituted C₂-C₆ alkenyl, optionally substituted C₁-C₆ heteroalkyl, optionally substituted C₃-C₁₀ cycloalkyl, optionally substituted 3- to 6-membered heterocyclyl, optionally substituted 5- to 10-membered heteroaryl, or optionally substituted C₆-C₁₀ aryl; and
R¹¹ is hydrogen, optionally substituted C₁-C₆ alkyl, optionally substituted C₁-C₆ heteroalkyl, optionally substituted C₂-C₆ alkenyl, optionally substituted C₂-C₆ heteroalkenyl, optionally substituted C₂-C₆ alkynyl, optionally substituted C₂-C₆ heteroalkynyl, optionally substituted C₃-C₁₀ cycloalkyl, optionally substituted 3- to 10-membered heterocyclyl, optionally substituted C₃-C₁₀ cycloalkenyl, optionally substituted 3- to 10-membered heterocycloalkenyl, optionally substituted C₆-C₁₀ aryl, or optionally substituted 5- to 10-membered heteroaryl.

18. The compound of claim 17, wherein the compound has the structure of Formula VIIa-2: 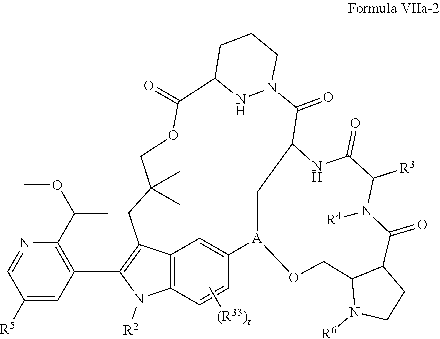
or a pharmaceutically acceptable salt thereof, wherein
R⁶ is hydrogen, optionally substituted C₁-C₆ alkyl, optionally substituted C₁-C₆ heteroalkyl, optionally substituted 3- to 6-membered heterocyclyl, optionally substituted 3- to 6-membered cycloalkyl, optionally substituted 5- to 10-membered heteroaryl, optionally substituted C₆-C₁₀ aryl, 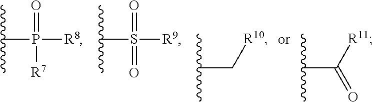
R⁷ and R⁸ are each, independently, H or optionally substituted C₁-C₆ alkyl;
R⁹ is optionally substituted C₁-C₆ alkyl, optionally substituted C₂-C₆ alkenyl, optionally substituted 3- to 6-membered cycloalkyl, or optionally substituted 3- to 6-membered heterocyclyl;
R¹⁰ is optionally substituted C₁-C₆ alkyl, optionally substituted C₂-C₆ alkenyl, optionally substituted C₁-C₆ heteroalkyl, optionally substituted C₃-C₁₀ cycloalkyl, optionally substituted 3- to 6-membered heterocyclyl, optionally substituted 5- to 10-membered heteroaryl, or optionally substituted C₆-C₁₀ aryl; and
R¹¹ is hydrogen, optionally substituted C₁-C₆ alkyl, optionally substituted C₁-C₆ heteroalkyl, optionally substituted C₂-C₆ alkenyl, optionally substituted C₂-C₆ heteroalkenyl, optionally substituted C₂-C₆ alkynyl, optionally substituted C₂-C₆ heteroalkynyl, optionally substituted C₃-C₁₀ cycloalkyl, optionally substituted 3- to 10-membered heterocyclyl, optionally substituted C₃-C₁₀ cycloalkenyl, optionally substituted 3- to 10-membered heterocycloalkenyl, optionally substituted C₆-C₁₀ aryl, or optionally substituted 5- to 10-membered heteroaryl.

19. A compound, or a pharmaceutically acceptable salt thereof, of Table 1 or Table 2.

20. A pharmaceutical composition comprising a compound, or a pharmaceutically acceptable salt thereof, of any one of claims 1-19 and a pharmaceutically acceptable excipient.

21. A method of treating cancer in a subject in need thereof, the method comprising administering to the subject a therapeutically effective amount of a compound, or a pharmaceutically acceptable salt thereof, of any one of claims 1-19 or a pharmaceutical composition of claim 20.

22. A method of treating a Ras protein-related disorder in a subject in need thereof, the method comprising administering to the subject a therapeutically effective amount of a compound of any one of claims 1-19, or a pharmaceutically acceptable salt thereof, or a pharmaceutical composition of claim 20.

23. A method of inhibiting a Ras protein in a cell, the method comprising contacting the cell with an effective amount of a compound of any one of claims 1 to 19, or a pharmaceutically acceptable salt thereof, or a pharmaceutical composition of claim 20.
